(19) United States
(12) Patent Application Publication
(10) Pub. No.: US 2014/0288040 A1
(43) Pub. Date: Sep. 25, 2014

(54) SUBSTITUTED BENZYLAMINE COMPOUNDS, THEIR USE IN MEDICINE, AND IN PARTICULAR THE TREATMENT OF HEPATITIS C VIRUS (HCV) INFECTION

(71) Applicant: ASTEX THERAPEUTICS LIMITED, Cambridge (GB)

(72) Inventors: Andrew James Woodhead, Cambridge (GB); Gianni Chessari, Cambridge (GB); Gilbert Ebai Besong, Bad Duerkheim (DE); Maria Grazia Carr, Luton (GB); Steven Douglas Hiscock, Royston (GB); Michael Alistair O'Brien, Hitchin (GB); David Charles Rees, Cambridge (GB); Susanne Maria Saalau-Bethell, Cambridge (GB); Hendrika Maria Gerarda Willems, Cambridge (GB); Neil Thomas Thompson, Cambridge (GB)

(73) Assignee: ASTEX THERAPEUTICS LIMITED, Cambridge (GB)

(21) Appl. No.: 14/355,780

(22) PCT Filed: Oct. 31, 2012
(86) PCT No.: PCT/EP2012/071560

§ 371 Date: May 1, 2014

Related U.S. Application Data
(60) Provisional application No. 61/554,415, filed on Nov. 1, 2011.
(60) Provisional application No. 61/645,283, filed on May 10, 2012.

(30) Foreign Application Priority Data
Nov. 1, 2011 (GB) 1118876.0

(51) Int. Cl. A61K 31/505; A61K 31/137; A61K 45/06; C07C 217/74; A61K 31/135; C07D 239/42; C07C 237/20; A61K 31/165; C07D 211/70; A61K 31/4409; C07D 295/027; A61K 31/4465; A61K 31/4462; A61K 31/397; C07C 217/58

(52) U.S. Cl. CPC A61K 31/505; C07C 217/58; A61K 31/137; A61K 45/06; C07C 217/74; A61K 31/135; C07D 239/42; C07C 237/20; A61K 31/165; C07D 211/70; A61K 31/4409; C07D 295/027; A61K 31/4465; A61K 31/4462; A61K 31/397
USPC 514/210.01; 564/430; 514/655; 564/306; 544/332; 514/275; 564/165; 514/620; 546/333; 514/357; 546/232; 514/331; 546/223; 514/329

(57) ABSTRACT

The invention provides compounds of the formula (6):

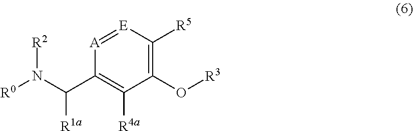

or a salt, N-oxide or tautomer thereof, wherein A is CH, CF or nitrogen; E is CH, CF or nitrogen; and R⁰ is hydrogen or C₁₋₂ alkyl;
R^{1a} is selected from CONH₂; CO₂H; an optionally substituted acyclic C₁₋₈ hydrocarbon group; and an optionally substituted monocyclic carbocyclic or heterocyclic group of 3 to 7 ring members, of which 0, 1, 2, 3 or 4 are heteroatom ring members selected from O, N and S;
R² is selected from hydrogen and a group R^{2a};
R^{2a} is selected from an optionally substituted acyclic C₁₋₈ hydrocarbon group; an optionally substituted monocyclic carbocyclic or heterocyclic group of 3 to 7 ring members, of which 0, 1 or 2 ring members are heteroatom ring members selected from O, N and S; and an optionally substituted bicyclic heterocyclic group of 9 or 10 ring members, of which 1 or 2 ring members are nitrogen atoms; wherein at least one of R¹ and R² is other than hydrogen;
R³ is an optionally substituted 3- to 10-membered monocyclic or bicyclic carbocyclic or heterocyclic ring containing 0, 1, 2 or 3 heteroatom ring members selected from N, O and S;
R^{4a} is selected from halogen; cyano; C₁₋₄ alkyl optionally substituted with one or more fluorine atoms; C₁₋₄ alkoxy optionally substituted with one or more fluorine atoms; hydroxy-C₁₋₄ alkyl; and C₁₋₂ alkoxy-C₁₋₄ alkyl;
R⁵ is selected from hydrogen and a substituent R^{5a}; and
R^{5a} is selected from C₁₋₂ alkyl optionally substituted with one or more fluorine atoms; C₁₋₃ alkoxy optionally substituted with one or more fluorine atoms; halogen; cyclopropyl; cyano; and amino.

The compounds have activity against hepatitis C virus and can be used in the prevention or treatment of hepatitis C viral infections.

### RELATED APPLICATIONS

This application is related to and claims the priority dates of UK patent application number GB1118876.0 filed on Nov. 1, 2011, U.S. provisional patent application No. 61554,415 filed on Nov. 1, 2011, and U.S. provisional application No. 61645,283 filed on May 10, 2012, the entire contents of each of which are incorporated herein by reference.

This invention relates to novel substituted benzylamine compounds, their use in medicine, and in particular the treatment of hepatitis C virus (HCV) infections. Also provided are pharmaceutical compositions containing the compounds and processes for making them.

### BACKGROUND OF THE INVENTION

Hepatitis C is a chronic liver disease affecting an estimated 3% of the global population, and is caused by the hepatitis C virus. Patients infected with the virus run an 85% risk of developing cirrhosis of the liver and of these, 20% will subsequently progress to hepatocellular carcinoma. HCV is recognized as a major cause of end-stage liver disease and the leading cause of liver transplantation in the developed world [Davila, J. A., et al. (2004) Gastroenterology, 127, 1372-1380; Liu, C. L. and Fan, S. T. (1997) Am. J. Surg., 173, 358-365; Garcia-Retortillo, M., et al. (2002) Hepatology, 35, 680-687; Brown, R. S. (2005) Nature, 436, 973-978]. Transplantation is not curative, since HCV-infected transplant recipients infect their donor livers. The disease burden and mortality related to HCV have risen substantially in the last decade and are predicted by the Centre for Disease Control and Prevention to increase further as the population infected, prior to widespread blood screening, ages.

The HCV genome encodes only 10 viral proteins, namely the structural proteins E1, E2 and C, and the non-structural proteins p7, NS2, NS3, NS4a, NS4b, NS5a and NS5b. The NS3 protein is a bi-functional enzyme with a serine protease domain at the N-terminus and an ATP dependent helicase domain at the C-terminus.

The nomenclature set forth in Simmonds et al., (1993) J Gen Virol, 74(Pt. 11):2391-2399 is widely used and classifies HCV isolates into six major genotypes 1 to 6 with two or more related subtypes, e.g., 1a, 1b. Additional genotypes 7-10 and 11 have been proposed but the phylogenetic basis on which this classification is based has been questioned, and thus type 7, 8, 9 and 11 isolates have been reassigned as type 6, and type 10 isolates as type 3 (see Lamballerie et al, J Gen Virol, 78(Pt.1):45-51 (1997)). The major genotypes have been defined as having sequence similarities of between 55 and 72% (mean 64.5%), and subtypes within types as having 75%-86% similarity (mean 80%) when sequenced in the NS5 region (see Simmonds et al., J Gen Virol, 75(Pt. 5):1053-1061 (1994)).

Of the six known genotypes of HCV, genotypes 1a and 1b are the most prevalent worldwide, followed by 3 and 6. The order of genotypic incidence in the UK is 3a (37.2%), 1a (30.7%), 1b (18.4%) and 2b (6.1%) which account for 92.4% of the reported cases, while in the USA 94.3% of reported infections are caused by the 1a (78.9%) and 1b (15.4%) genotypes [HCV database website at http:/hcv.lanl.gov].

The standard therapy for HCV is under review following the approval of telaprevir and boceprevir. The nature and duration of the is dependent on which genotype being treated. For the treatment of infection with HCV genotype 4, the treatment regime remains a combination of weekly injections of pegylated interferon α and daily oral administration of ribavirin for a period of 48 weeks. For the treatment of infection by HCV genotype 1, the treatment regime comprises the administration of pegylated interferon α and the twice daily oral administration of ribavirin plus the three times daily oral administration of telapravir or boceprevir. For the treatment of infection by HCV genotypes 2 and 3, the treatment regime comprises the administration of pegylated interferon α and twice daily oral administration of 400 mg of ribavirin for twenty four weeks. The treatment of HCV infections is costly and is associated with numerous severe side effects, including psychiatric disorders (depression, headaches), neutropaenia, pancreatitis, diabetes, hypersensitivity reactions, haemolytic anaemia and fatigue. Ribavirin has been shown to be teratogenic in all animals tested and is contraindicated during pregnancy. Moreover, according to NICE, the treatment with pegylated interferon α ribavirin is only successful in 54-56% of patients infected with the 1a and 1b genotypes, leaving a large group of patients with no treatment alternatives.

Host genetic factors have been found to influence treatment outcome. In particular, a single nucleotide polymorphism (SNP) on chromosome 19, rs1297980, has been shown to have a strong association with response to current standard of care. Patients with the CC genotype of rs1297980 had greater than two-fold likelyhood to achieve SVR than patients with non CC genotype infected with genotype 1 HCV (Ge et al., Nature 2009; 461:399-401). The trend was also evident in patients infected with GT2 and 3, though the effect was attenuated (Mangia et al, Gastroenterology (2010) 139(3):821-7).

The approval in the US and the European Union of the two NS34a active site protease inhibitors, telaprevir and boceprevir, is providing more treatment options to patients, with the National Institute for Clinical Excellence (NICE) issuing guidelines for their use. Both compounds show dramatic and sustained decreases in viral RNA levels in patients, but suffer from poor PK profiles and require high dosing regimes twice or thrice daily. In addition, both compounds lead to the emergence of resistance mutations [Sarrazin, C., et al. (2007) Gastroenterology, 132, 1767-1777; Kim, A. Y. and Timm, J. (2008) Expert Rev Anti Infect Ther., 6, 463-478]. As both compounds bind in the same region of the protease enzyme, mutants demonstrate cross resistance. Alternative therapies based on other HCV molecular targets, as well as second wave and second generation protease inhibitors are at earlier stages in clinical trials. Clinical experience suggests that emerging resistance is likely to be a major problem with most agents, with the possible exception of nucleot(s)ide based inhibitors of NS5b polymerase [Le Pogam, S., et al. (2010) J. Infect Dis. 202, 1510-9]. First-line therapies are likely to be combinations of effective agents that demonstrate differential cross resistance [Sarrazin, C. and Zeuzem, S (2010) Gastroenterology, 138, 447-462].

Inhibition of the NS34a protease activity by small active site directed molecules has been shown to halt viral replication in vitro, in the replicon cell-based assay, in the chimeric mouse model and most importantly in the clinic [Lin, C., et al. (2006) Infect Disord Drug Targets. 6, 3-16; Venkatraman, S., et al. (2006) J. Med. Chem. 49, 6074-6086; Zhou, Y., et al. (2007) J. Biol. Chem. 282, 22619-22628; Prongay, A. J., et al. (2007) J. Med. Chem. 50, 2310-2318; and Hezode, C., et al. (2009) N. Engl. J. Med. 360, 1839-49.

The HCV NS3 NTPasehelicase functions have also been extensively studied and are considered as potential targets for antiviral therapy [Frick, D. N. (2007) Curr. Issues Mol. Biol., 9, 1-20; Serebrov, V., et al. (2009) J. Biol. Chem., 284 (4), 2512-21. However, no agents are reported to be in clinical development (Swan T. and Kaplan, K. (2012) Hepatitis C Drug Development Goes from Pony Ride to Rocket Launch—The pipeline report 2012 at http:/www.pipelinereport.orgtocHCV).

Agents that inhibit helicase function by competing with the nucleic acid substrate have also been reported [Maga, G., et al. (2005) Biochem., 44, 9637-44]. A recent publication by the group of A. M. Pyle, suggests that the full length NS3 protein must undergo a conformational change to facilitate the formation of the functional complex between the enzyme and substrate RNA [Ding, S. C., et al. (2011) J. Virol., 85(9) 4343-4353]. They propose that an extended conformation, also necessary to allow access of substrates to the protease active site, represents the functionally active form of the full length protein for RNA unwinding. Further support for the extended conformation and protease domain interaction with RNA comes from a study that reports the specific interaction of viral RNA with the NS3 protease active site [Vaughan, R. et al. (2012) Virus Research, 169(1), 80-90, RNA binding by the NS3 protease of the hepatitis C virus, available on line at http:/dx.doi.org10.1016j.virusres.2012.07.007].

Jhoti et al. Nature Chemical Biology, 2012, doi:10.1038nchembio.1081, available online (the entire contents of which are incorporated herein by reference) reports the discovery of a highly conserved novel binding site located at the interface between the protease and helicase domains of the Hepatitis C Virus (HCV) NS3 protein. This site is reported to have a regulatory function on the protease activity via an allosteric mechanism. Jhoti et al. propose that compounds binding at this allosteric site inhibit the function of the NS3 protein by stabilising an inactive conformation and thus represent a new class of direct acting antiviral agents.

### SUMMARY OF THE INVENTION

The present invention provides compounds which are useful in the prevention or treatment of hepatitis C virus (HCV) infection.

Accordingly, in a first embodiment (Embodiment 1.0), the invention provides a compound for use in the prevention or treatment of a viral infection, wherein the compound has the formula (0):

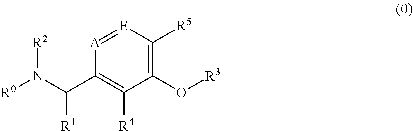

or a salt, N-oxide or tautomer thereof, wherein:
A is CH, CF or nitrogen;
E is CH, CF or nitrogen;
R⁰ is hydrogen or C₁₋₂ alkyl;
R¹ is selected from hydrogen and a group R^{1a}:
R^{1a} is selected from;
   CONH₂;
   CO₂H;
   an acyclic C₁₋₈ hydrocarbon group optionally substituted with one or two substituents R⁶, wherein one carbon atom of the acyclic C₁₋₈ hydrocarbon group may optionally be replaced by a heteroatom or group selected from O, S, NR^{c}, S(O) and SO₂, or two adjacent carbon atoms of the acyclic C₁₋₈ hydrocarbon group may optionally be replaced by a group selected from CONR^{c}, NR^{c}CO, NR^{c}SO₂ and SO₂NR^{c} provided that in each case at least one carbon atom of the acyclic C₁₋₈ hydrocarbon group remains; and
   a monocyclic carbocyclic or heterocyclic group of 3 to 7 ring members, of which 0, 1, 2, 3 or 4 are heteroatom ring members selected from O, N and S, the carbocyclic or heterocyclic group being optionally substituted with one or two substituents R^{7a};
R² is selected from hydrogen and a group R^{2a};
R^{2a} is selected from an acyclic C₁₋₈ hydrocarbon group optionally substituted with one or two substituents R⁸ wherein one carbon atom of the acyclic C₁₋₈ hydrocarbon group may optionally be replaced by a heteroatom or group selected from O and NR^{c} provided that at least one carbon atom of the acyclic C₁₋₈ hydrocarbon group remains; a monocyclic carbocyclic or heterocyclic group of 3 to 7 ring members, of which 0, 1 or 2 ring members are heteroatom ring members selected from O, N and S; and a bicyclic heterocyclic group of 9 or 10 ring members, of which 1 or 2 ring members are nitrogen atoms, one of the rings of the bicyclic heterocyclic group being a non-aromatic nitrogen-containing ring; the monocyclic carbocyclic or heterocyclic group and the bicyclic heterocyclic group each being optionally substituted with one or two substituents R^{7b};
   wherein at least one of R¹ and R² is other than hydrogen;
R³ is a 3- to 10-membered monocyclic or bicyclic carbocyclic or heterocyclic ring containing 0, 1, 2 or 3 heteroatom ring members selected from N, O and S, and being optionally substituted with one or more substituents R¹³;
R⁴ is selected from hydrogen and a substituent R^{4a};
R^{4a} is selected from halogen; cyano; C₁₋₄ alkyl optionally substituted with one or more fluorine atoms; C₁₋₄ alkoxy optionally substituted with one or more fluorine atoms; hydroxy-C₁₋₄ alkyl; and C₁₋₂ alkoxy-C₁₋₄ alkyl;
R⁵ is selected from hydrogen and a substituent Rya;
R^{5a} is selected from C₁₋₂ alkyl optionally substituted with one or more fluorine atoms; C₁₋₃ alkoxy optionally substituted with one or more fluorine atoms; halogen; cyclopropyl; cyano; and amino;
R⁶ is selected from hydroxy; fluorine; carbamoyl; mono- or di-C₁₋₄ alkylcarbamoyl; nitro; amino; mono- or di-C₁₋₄ alkylamino; a monocyclic carbocyclic or heterocyclic group of 3 to 7 ring members, of which 0, 1 or 2 are heteroatom ring members selected from O, N and S, the carbocyclic or heterocyclic group being optionally substituted with one or two substituents R^{7c};
R^{7a}, R^{7b}, R^{7c}, R^{7d}, R^{7e} and R^{7f} are each independently selected from oxo; amino; halogen; cyano; hydroxy; C₁₋₄ alkyl; hydroxy-C₁₋₄ alkyl; amino-C₁₋₄alkyl; mono- and di-C₁₋₄ alkylamino-C₁₋₄ alkyl;
R⁸ is selected from hydroxy; halogen; cyano; C(═NH)NHR⁹; C(═O)NR¹⁰R¹¹; amino; mono- or di-C₁₋₄ alkylamino; a non-aromatic monocyclic carbocyclic or heterocyclic group of 3 to 7 ring members, of which 0, 1 or 2 are heteroatom ring members selected from O, N and S, the non-aromatic monocyclic carbocyclic or heterocyclic group being optionally substituted with 1 or 2 substituents R^{7d}; and an aromatic heterocyclic group selected from pyrrole, imidazole, pyrazole, indole and pyridone, the aromatic heterocyclic group being optionally substituted with 1 or 2 substituents R^{7e}; provided that the carbon atom of the acyclic C₁₋₈ hydrocarbon group which is attached directly to the moiety NR⁰ cannot be substituted with hydroxy or an N-linked substituent;
R⁹ is selected from hydrogen, C₁₋₄ alkyl and C₁₋₄ alkanoyl;
R¹⁰ is selected from hydrogen and C₁₋₄ alkyl;
R¹¹ is selected from hydrogen; hydroxy; C₁₋₄ alkoxy; amino; mono- or di-C₁₋₄ alkylamino; a non-aromatic monocyclic carbocyclic or heterocyclic group of 3 to 7 ring members, of which 0, 1 or 2 are heteroatom ring members selected from O, N and S, the non-aromatic monocyclic carbocyclic or heterocyclic group being optionally substituted with one or two substituents R^{7f}; and C₁₋₆ alkyl, wherein the C₁₋₆ alkyl is optionally substituted with 1, 2 or 3 substituents R¹²;

or NR¹⁰R¹¹ forms a non-aromatic heterocyclic ring having a total of 4 to 7 ring members of which 1 or 2 are nitrogen atoms and the others are carbon atoms, the said non-aromatic heterocyclic ring being optionally substituted with one or more substituents selected from hydroxy, amino and C₁₋₄ alkyl;
R¹² is selected from hydroxy; C₁₋₄ alkoxy; cyano; C₁₋₄alkoxycarbonyl; amino; mono- or di-C₁₋₄ alkylamino; C₃₋₆cycloalkylamino; CONH₂; CONH(C₁₋₄alkyl); CON(C₁₋₄alkyl)₂ and a group —NH—CH₂-Cyc; where Cyc is a benzene, furan, thiophene or pyridine ring;
R¹³ is selected from halogen; cyano; nitro; CH═NOH; and a group R^{a}-R^{b}; and is optionally further selected from oxo;
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is hydrogen; a cyclic group R^{d}; or an acyclic C₁₋₈ hydrocarbon group optionally substituted with one or more substituents selected from hydroxy, oxo, halogen, cyano, nitro, carboxy, amino, mono- or di-C₁₋₄ alkylamino, and a cyclic group R^{d}; wherein one or two but not all of the carbon atoms of the acyclic C₁₋₈ hydrocarbon group may optionally be replaced by O, S, SO, SO₂, NR^{c}, X¹C(X²), C(X²)X¹ or X¹C(X²)X¹; SO₂NR^{c} or NR^{c}SO₂;
the cyclic group R^{d} is a monocyclic carbocyclic or heterocyclic group having from 3 to 7 ring members, of which 0, 1, 2 or 3 are heteroatom ring members selected from O, N and S and oxidised forms thereof, the carbocyclic or heterocyclic group being optionally substituted with one or more substituents selected from R¹⁴; but excluding the combination wherein R^{a} is a bond and R^{b} is hydrogen;

R¹⁴ is selected from oxo; halogen; cyano; and R^{a}-R^{e};

R^{e} is hydrogen or an acyclic C₁₋₈ hydrocarbon group optionally substituted with one or more substituents selected from phenyl; hydroxy; oxo; halogen; cyano; carboxy; amino; mono- or di-C₁₋₄ alkylamino; wherein one or two but not all of the carbon atoms of the acyclic C₁₋₈ hydrocarbon group may optionally be replaced by O, S, SO, SO₂, NR^{c}, X¹C(X²), C(X²)X¹ or X¹C(X²)X¹; SO₂NR^{c} or NR^{c}SO₂;
X¹ is O or NR^{c};
X² is ═O or ═NR^{c}; and
R^{c} is hydrogen or C₁₋₄ alkyl.

In another embodiment (Embodiment 1.00), the invention provides a compound of the formula (0) according to Embodiment 1.0 for use in the prevention or treatment of hepatitis C virus (HCV) infections.

In a further embodiment (Embodiment 1.1), the invention provides a compound of the formula (1):

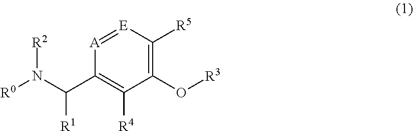

or a salt, N-oxide or tautomer thereof, wherein:
A is CH, CF or nitrogen;
E is CH, CF or nitrogen;
R⁰ is hydrogen or C₁₋₂ alkyl;
R¹ is selected from hydrogen and a group R^{1a}
R^{1a} is selected from;
   CONH₂;
   CO₂H;
   an acyclic C₁₋₈ hydrocarbon group optionally substituted with one or two substituents R⁶, wherein one carbon atom of the acyclic C₁₋₈ hydrocarbon group may optionally be replaced by a heteroatom or group selected from O, S, NR^{c}, S(O) and SO₂, or two adjacent carbon atoms of the acyclic C₁₋₈ hydrocarbon group may optionally be replaced by a group selected from CONR^{c}, NR^{c}CO, NR^{c}SO₂ and SO₂NR^{c} provided that in each case at least one carbon atom of the acyclic C₁₋₈ hydrocarbon group remains; and
   a monocyclic carbocyclic or heterocyclic group of 3 to 7 ring members, of which 0, 1, 2, 3 or 4 are heteroatom ring members selected from O, N and S, the carbocyclic or heterocyclic group being optionally substituted with one or two substituents R^{7a};
R² is selected from hydrogen and a group R^{2a};
R^{2a} is selected from an acyclic C₁₋₈ hydrocarbon group optionally substituted with one or two substituents R⁸ wherein one carbon atom of the acyclic C₁₋₈ hydrocarbon group may optionally be replaced by a heteroatom or group selected from O and NR^{c} provided that at least one carbon atom of the acyclic C₁₋₈ hydrocarbon group remains; a monocyclic carbocyclic or heterocyclic group of 3 to 7 ring members, of which 0, 1 or 2 ring members are heteroatom ring members selected from O, N and S; and a bicyclic heterocyclic group of 9 or 10 ring members, of which 1 or 2 ring members are nitrogen atoms, one of the rings of the bicyclic heterocyclic group being a non-aromatic nitrogen-containing ring; the monocyclic carbocyclic or heterocyclic group and the bicyclic heterocyclic group each being optionally substituted with one or two substituents R^{7b};
   wherein at least one of R¹ and R² is other than hydrogen;
R³ is a 3- to 10-membered monocyclic or bicyclic carbocyclic or heterocyclic ring containing 0, 1, 2 or 3 heteroatom ring members selected from N, O and S, and being optionally substituted with one or more substituents R¹³;
R⁴ is selected from hydrogen and a substituent R^{4a};
R^{4a} is selected from halogen; cyano; C₁₋₄ alkyl optionally substituted with one or more fluorine atoms; C₁₋₄ alkoxy optionally substituted with one or more fluorine atoms; hydroxy-C₁₋₄ alkyl; and C₁₋₂ alkoxy-C₁₋₄ alkyl;
R⁵ is selected from hydrogen and a substituent R^{5a};
R^{5a} is selected from C₁₋₂ alkyl optionally substituted with one or more fluorine atoms; C₁₋₃ alkoxy optionally substituted with one or more fluorine atoms; halogen; cyclopropyl; cyano; and amino;
R⁶ is selected from hydroxy; fluorine; carbamoyl; mono- or di-C₁₋₄ alkylcarbamoyl; nitro; amino; mono- or di-C₁₋₄ alkylamino; a monocyclic carbocyclic or heterocyclic group of 3 to 7 ring members, of which 0, 1 or 2 are heteroatom ring members selected from O, N and S, the carbocyclic or heterocyclic group being optionally substituted with one or two substituents R^{7c};
R^{7a}, R^{7b}, R^{7c}, R^{7d}, R^{7e} and R^{7f} are each independently selected from oxo; amino; halogen; cyano; hydroxy; C₁₋₄ alkyl; hydroxy-C₁₋₄ alkyl; amino-C₁₋₄ alkyl; mono- and di-C₁₋₄ alkylamino-C₁₋₄ alkyl;
R⁸ is selected from hydroxy; halogen; cyano; C(═NH)NHR⁹; C(═O)NR¹⁰R¹¹; amino; mono- or di-C₁₋₄ alkylamino; a non-aromatic monocyclic carbocyclic or heterocyclic group of 3 to 7 ring members, of which 0, 1 or 2 are heteroatom ring members selected from O, N and S, the non-aromatic monocyclic carbocyclic or heterocyclic group being optionally substituted with 1 or 2 substituents R^{7d}; and an aromatic heterocyclic group selected from pyrrole, imidazole, pyrazole, indole and pyridone, the aromatic heterocyclic group being optionally substituted with 1 or 2 substituents R^{7e}; provided that the carbon atom of the acyclic C₁₋₈ hydrocarbon group which is attached directly to the moiety NR⁰ cannot be substituted with hydroxy or an N-linked substituent;
R⁹ is selected from hydrogen, C₁₋₄ alkyl and C₁₋₄ alkanoyl;
R¹⁰ is selected from hydrogen and C₁₋₄ alkyl;
R¹¹ is selected from hydrogen; hydroxy; C₁₋₄ alkoxy; amino; mono- or di-C₁₋₄ alkylamino; a non-aromatic monocyclic carbocyclic or heterocyclic group of 3 to 7 ring members, of which 0, 1 or 2 are heteroatom ring members selected from O, N and S, the non-aromatic monocyclic carbocyclic or heterocyclic group being optionally substituted with one or two substituents R^{7f}; and C₁₋₆ alkyl, wherein the C₁₋₆ alkyl is optionally substituted with 1, 2 or 3 substituents R¹²;
or NR¹⁰R¹¹ forms a non-aromatic heterocyclic ring having a total of 4 to 7 ring members of which 1 or 2 are nitrogen atoms and the others are carbon atoms, the said non-aromatic heterocyclic ring being optionally substituted with one or more substituents selected from hydroxy, amino and C₁₋₄ alkyl;
R¹² is selected from hydroxy; C₁₋₄ alkoxy; cyano; C₁₋₄alkoxycarbonyl; amino; mono- or di-C₁₋₄ alkylamino; C₃₋₆cycloalkylamino; CONH₂; CONH(C₁₋₄alkyl); CON(C₁₋₄alkyl)₂ and a group —NH—CH₂-Cyc; where Cyc is a benzene, furan, thiophene or pyridine ring;
R¹³ is selected from halogen; cyano; nitro; CH═NOH; and a group R^{a}-R^{b}; and is optionally further selected from oxo;
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is hydrogen; a cyclic group R^{d}; or an acyclic C₁₋₈ hydrocarbon group optionally substituted with one or more substituents selected from hydroxy, oxo, halogen, cyano, nitro, carboxy, amino, mono- or di-C₁₋₄ alkylamino, and a cyclic group R^{d}; wherein one or two but not all of the carbon atoms of the acyclic C₁₋₈ hydrocarbon group may optionally be replaced by O, S, SO, SO₂, NR^{c}, X¹C(X²), C(X²)X¹ or X¹C(X²)X¹; SO₂NR^{c} or NR^{c}SO₂;
the cyclic group R^{d} is a monocyclic carbocyclic or heterocyclic group having from 3 to 7 ring members, of which 0, 1, 2 or 3 are heteroatom ring members selected from O, N and S and oxidised forms thereof, the carbocyclic or heterocyclic group being optionally substituted with one or more substituents selected from R¹⁴; but excluding the combination wherein R^{a} is a bond and R^{b} is hydrogen;
R¹⁴ is selected from oxo; halogen; cyano; and R^{a}-R^{e};
R^{e} is hydrogen or an acyclic C₁₋₈ hydrocarbon group optionally substituted with one or more substituents selected from phenyl; hydroxy; oxo; halogen; cyano; carboxy; amino; mono- or di-C₁₋₄ alkylamino; wherein one or two but not all of the carbon atoms of the acyclic C₁₋₈ hydrocarbon group may optionally be replaced by O, S, SO, SO₂, NR^{c}, X¹C(X²), C(X²)X¹ or X¹C(X²)X¹; SO₂NR^{c} or NR^{c}SO₂;
X¹ is O or NR^{c};
X² is ═O or ═NR^{c}; and
R^{c} is hydrogen or C₁₋₄ alkyl;
   with the provisos that:
   (i) when R³ is phenyl, A and E are both CH, R⁴ and R⁵ are both hydrogen, R⁰ is hydrogen and R¹ is CONH₂, then R² is other than ethyl or propyl;
   (ii) when R³ is 4-chlorophenyl, A and E are both CH, R⁴ and R⁵ are both hydrogen, R⁰ is hydrogen and R¹ is 2-hydroxyethyl, then R² is other than ethyl;
   (iii) when R³ is phenyl, A and E are both CH, R⁴ and R⁵ are both hydrogen, R⁰ is hydrogen and R¹ is 2-hydroxymethyl, then R² is other than ethyl, propyl, isobutyl and cyclopropylmethyl;
   (iv) when R³ is phenyl, A and E are both CH, R⁴ and R⁵ are both hydrogen, R⁰ is hydrogen and R¹ is cyano, then R² is other than ethyl, propyl and cyclopropylmethyl;
   (v) when R³ is phenyl, A and E are both CH, R⁴ and R⁵ are both hydrogen, R⁰ and R² are both hydrogen, then R¹ is other than ethyl;
   (vi) when R³ pyrimidin-2-yl or 4-chlorophenyl, R⁴ and R⁵ are both hydrogen, R¹ is hydrogen, R² is R^{2a} wherein R^{2a} is an acyclic C₁₋₈ hydrocarbon group substituted with one or two substituents R⁸, then at least one substituent R⁸ is C(═O)NR¹⁰R¹¹;
   (vi) when R³ is pyridin-3-yl, pyridine-4-yl, or phenyl, R⁴ and R⁵ are both hydrogen, R¹ is hydrogen, R² is R^{2a} wherein R^{2a} is —CH₂CH₂—R⁸, then R⁸ is other than an unsubstituted or substituted indole;
   (vii) when A is N, R³ is a substituted benzoimidazole group, R⁴ and R⁵ are both hydrogen, R¹ is hydrogen, R² is R^{2a} wherein R^{2a} is an acyclic C₁₋₈ hydrocarbon group substituted with one or two substituents R⁸, then at least one substituent R⁸ is C(═O)NR¹⁰R¹¹;
   (vii) when R³ is pyrimidin-2-yl, 5-bromo-pyrimidin-2-yl, phenyl, 4-methoxyphenyl, 4-nitro-2-methoxycarbonylphenyl, a substituted imidazopyridazine or 4-chlorophenyl, R⁴ and R⁵ are both hydrogen, R⁰ is hydrogen or C₁₋₂ alkyl, R¹ is R^{1a} wherein R^{1a} is methyl or hydroxymethyl and R² is R^{2a}, then R^{2a} is other than C₁₋₄ alkyl or cyclopropylmethyl;
   (viii) when R³ is phenyl, R⁴ and R⁵ are both hydrogen, R⁰ is hydrogen or C₁₋₂ alkyl, R¹ is R^{1a} wherein R^{1a} is CO₂H, CONH₂ or CH₂NH₂, and R² is R^{2a}, then R^{2a} is other than C₁₋₄ alkyl or hydroxyethyl;
   (ix) when R³ is 4-chlorophenyl, R⁴ and R⁵ are both hydrogen, R⁰ is hydrogen, R¹ is R^{1a} wherein R^{1a} is hydroxyethyl and R² is R^{2a}, then R^{2a} is other than C₁₋₂ alkyl;
   (x) when R³ is phenyl, R⁴ and R⁵ are both hydrogen, R⁰ is hydrogen or C₁₋₂ alkyl, R¹ is R^{1a} wherein R^{1a} is a cyclohexane group, and R² is R^{2a}, then R^{2a} is other than methyl; and
   (xi) when R⁰ and R² are both methyl, R— is R^{1a} where R^{1a} is phenyl, R⁴ is hydrogen and R⁵ is methoxy, then R³ is other than phenyl bearing a substituent —CH(NMe₂)-Ph at the para position thereof.

Particular and preferred compounds of the formula (1) are as defined in the Embodiments 1.2 to 1.109 below.

1.2 A compound according to Embodiment 1.1 wherein A is CH or CF.

1.2A A compound according to Embodiment 1.2 wherein A is CH.

1.2B A compound according to Embodiment 1.2 wherein A is CF.

1.2C A compound according to Embodiment 1.1 wherein A is N.

1.3 A compound according to Embodiment 1.1 or Embodiment 1.2 wherein E is CH or CF.

1.3A A compound according to Embodiment 1.3 wherein E is CH.

1.3B A compound according to Embodiment 1.1 or 1.2 wherein E is CF.

1.3C A compound according to any one of Embodiments 1.1 and 1.2 to 1.2C wherein E is N.

1.4 A compound according to any one of Embodiments 1.1 to 1.3C wherein R⁰ is hydrogen.

1.5 A compound according to any one of Embodiments 1.1 to 1.3C wherein R⁰ is C₁₋₂ alkyl.

1.6 A compound according to Embodiment 1.5 wherein R⁰ is methyl.

1.7 A compound according to Embodiment 1.5 wherein R⁰ is ethyl.

1.8 A compound according to any one of Embodiments 1.1 to 1.7 wherein R¹ is selected from hydrogen and a group R^{1a} wherein R^{1a} is selected from;
CONH₂;
an acyclic C₁₋₈ hydrocarbon group optionally substituted with one or two substituents R⁶, wherein one carbon atom of the acyclic C₁₋₈ hydrocarbon group may optionally be replaced by a heteroatom or group selected from O, S, NR^{c}, S(O) and SO₂, or two adjacent carbon atoms of the acyclic C₁₋₈ hydrocarbon group may optionally be replaced by a group selected from CONR^{c}, NR^{c}CO, NR^{c}SO₂ and SO₂NR^{c} provided that in each case at least one carbon atom of the acyclic C₁₋₈ hydrocarbon group remains; and
a monocyclic carbocyclic or heterocyclic group of 3 to 7 ring members, of which 0, 1 or 2 are heteroatom ring members selected from O, N and S, the carbocyclic or heterocyclic group being optionally substituted with one or two substituents R^{7a}.

1.8A A compound according to any one of Embodiments 1.1 to 1.8 wherein R¹ is selected from hydrogen and a group R^{1a} wherein R^{1a} is selected from:
an acyclic C₁₋₈ hydrocarbon group optionally substituted with one substituent R⁶, wherein one carbon atom of the acyclic C₁₋₈ hydrocarbon group may optionally be replaced by a heteroatom O; and
a monocyclic carbocyclic or heterocyclic group of 3, 4, 5 or 6 ring members, of which 0, 1 or 2 are heteroatom ring members selected from O and N, the carbocyclic or heterocyclic group being optionally substituted with one or two substituents R^{7a}.

1.9 A compound according to Embodiment 1.8A wherein R¹ is selected from hydrogen and a group R^{1a} wherein R^{1a} is selected from:
an acyclic C₁₋₈ hydrocarbon group optionally substituted with one substituent R⁶, wherein one carbon atom of the acyclic C₁₋₈ hydrocarbon group may optionally be replaced by a heteroatom O;
a monocyclic carbocyclic group of 3, 4, 5 or 6 members, the monocyclic carbocyclic group being optionally substituted with one or two substituents R^{7a}; and
a monocyclic heterocyclic group of 5 or 6 ring members, of which 1 or 2 are nitrogen atoms, the monocyclic heterocyclic group being optionally substituted with one or two substituents R^{7a}.

1.10 A compound according to Embodiment 1.9 wherein R¹ is selected from hydrogen and a group R^{1a} wherein R^{1a} is selected from:
an acyclic C₁₋₈ hydrocarbon group optionally substituted with one substituent R⁶, wherein one carbon atom of the acyclic C₁₋₈ hydrocarbon group may optionally be replaced by a heteroatom O;
a monocyclic carbocyclic group of 3 ring members; and
a monocyclic heterocyclic group of 6 ring members, of which 1 is a nitrogen atom, the monocyclic heterocyclic group being optionally substituted with one or two substituents R^{7a}.

1.11 A compound according to either of Embodiments 1.9 and 1.10 wherein the monocyclic heterocyclic group is unsubstituted.

1.12 A compound according to any one of Embodiments 1.8 to 1.11 wherein the substituent R⁶ is a monocyclic heterocyclic group of 5 or 6 ring members, of which 1 or 2 are nitrogen atoms, the heterocyclic group being optionally substituted with one or two substituents R^{7c}.

1.13 A compound according to Embodiment 1.12 wherein the substituent R⁶ is a monocyclic heterocyclic group of 6 ring members, of which 1 is a nitrogen atom, the monocyclic heterocyclic group being optionally substituted with one or two substituents R^{7c}.

1.14 A compound according to either of Embodiments 1.12 and 1.13 wherein the monocyclic heterocyclic group is unsubstituted or substituted with one substituent R^{7c}.

1.15 A compound according to any one of Embodiments 1.8 to 1.14 wherein the acyclic hydrocarbon group is an acyclic C₁₋₆ hydrocarbon group; and one carbon atom of the acyclic C₁₋₆ hydrocarbon group may optionally be replaced by a heteroatom O.

1.16 A compound according to Embodiment 1.15 wherein the acyclic hydrocarbon group is an acyclic C₁₋₅ hydrocarbon group; and one carbon atom of the acyclic C₁₋₅ hydrocarbon group may optionally be replaced by a heteroatom O.

1.17 A compound according to Embodiment 1.16 wherein the acyclic hydrocarbon group is an acyclic C₁₋₄ hydrocarbon group, and one carbon atom of the acyclic C₁₋₄ hydrocarbon group may optionally be replaced by a heteroatom O.

1.18 A compound according to any one of Embodiments 1.8 to 1.17 wherein the acyclic hydrocarbon group is an alkyl group wherein one carbon atom of the alkyl group may optionally be replaced by a heteroatom O.

1.18A A compound according to Embodiment 1.8 wherein R¹ is selected from hydrogen and a group R^{1a} wherein R^{1a} is selected from a piperidine group; a cyclopropyl group; and a C₁₋₆ alkyl group optionally substituted with a piperidine group; and wherein one carbon atom of the C₁₋₄ alkyl group may optionally be replaced by a heteroatom O.

1.18B A compound according to Embodiment 1.18B wherein R¹ is selected from hydrogen and a group R^{1a} wherein R^{1a} is selected from a piperidin-4-yl group; a cyclopropyl group; and a C₁₋₆ alkyl group optionally substituted with a piperidin-4-yl group; and wherein one carbon atom of the C₁₋₆ alkyl group may optionally be replaced by a heteroatom O.

1.18C A compound according to Embodiment 1.18B wherein R¹ is a group R^{1a} wherein R^{1a} is ethyl, cyclopropyl, 3-pentyl or methoxyethyl.

1.18D A compound according to Embodiment 1.18C wherein R^{1a} is 3-pentyl.

1.19 A compound according to Embodiment 1.8 wherein R¹ is selected from hydrogen and a group R^{1a} wherein R^{1a} is selected from a piperidine group; a cyclopropyl group; and a C₁₋₄ alkyl group optionally substituted with a piperidine group; and wherein one carbon atom of the C₁₋₄ alkyl group may optionally be replaced by a heteroatom O.

1.20 A compound according to Embodiment 1.19 wherein R¹ is selected from hydrogen and a group R^{1a} wherein R^{1a} is selected from a piperidin-4-yl group; a cyclopropyl group; and a C₁₋₄ alkyl group optionally substituted with a piperidin-4-yl group; and wherein one carbon atom of the C₁₋₄ alkyl group may optionally be replaced by a heteroatom O.

1.21 A compound according to Embodiment 1.20 wherein R¹ is selected from hydrogen and a group R^{1a} wherein R^{1a} is selected from; a piperidin-4-yl group; cyclopropyl; an unsubstituted C₁₋₄ alkyl group wherein one carbon atom of the C₁₋₄ alkyl group may optionally be replaced by a heteroatom O; and a substituted C₁₋₃ alkyl group wherein the substituent is a piperidin-4-yl group.

1.22 A compound according to Embodiment 1.21 wherein R¹ is a group R^{1a} wherein R^{1a} is ethyl, cyclopropyl or methoxyethyl.

1.22A A compound according to Embodiment 1.22 wherein R^{1a} is ethyl.

1.22B A compound according to Embodiment 1.22 wherein R^{1a} cyclopropyl.

122C. A compound according to Embodiment 1.22 wherein R^{1a} is methoxyethyl.

1.23 A compound according to Embodiment 1.21 wherein R¹ is hydrogen.

1.24 A compound according to any one of Embodiments 1.1 to 1.22 wherein R¹ is a group R^{1a}.

1.25 A compound according to any one of Embodiments 1.1 to 1.24 wherein R² is selected from hydrogen and a group R^{2a} wherein R^{2a} is selected from an acyclic C₁₋₈ hydrocarbon group optionally substituted with one or two substituents R⁸; a monocyclic carbocyclic or heterocyclic group of 5 or 6 ring members, of which 0, 1 or 2 ring members are heteroatom ring members selected from O and N; and a bicyclic heterocyclic group of 9 or 10 ring members, of which 1 or 2 ring members are nitrogen atoms, one of the rings of the bicyclic heterocyclic group being a benzene ring and the other of the rings being a 5 or 6 membered non-aromatic heterocyclic ring; the monocyclic carbocyclic or heterocyclic group and the bicyclic heterocyclic group each being optionally substituted with one or two substituents R^{7b};

1.26 A compound according to Embodiment 1.26 wherein R² is selected from hydrogen and R^{2a} wherein R^{2a} is selected from a C₁₋₈ alkyl group optionally substituted with one or two substituents R⁸; a monocyclic carbocyclic or heterocyclic group of 4 to 6 ring members selected from C₄₋₆ cycloalkyl, imidazole, piperidine, pyridine and tetrahydropyridine; and a bicyclic heterocyclic group of 9 or 10 ring members, one of the rings of the bicyclic heterocyclic group being a benzene ring and the other of the rings being a 5 or 6 membered non-aromatic heterocyclic ring containing a single heteroatom ring member which is nitrogen; the monocyclic carbocyclic or heterocyclic group and the bicyclic heterocyclic group each being optionally substituted with one or two substituents R^{7b}.

1.27 A compound according to any one of Embodiments 1.1 to 1.26 wherein the optional substituents R⁸ are selected from hydroxy; halogen; amino; C(═NH)NHR⁹; C(═O)NR¹⁰R¹¹; a non-aromatic monocyclic carbocyclic or heterocyclic group of 3 to 6 ring members, of which 0, 1 or 2 are heteroatom ring members selected from O and N, the carbocyclic or heterocyclic group being optionally substituted with 1 or 2 substituents R^{7d}; and an aromatic heterocyclic group selected from pyrrole, imidazole, pyrazole, indole and pyridone, the aromatic heterocyclic group being optionally substituted with 1 or 2 substituents R^{7e}.

1.27A A compound according to any one of Embodiments 1.1 to 1.26 wherein the optional substituents R⁸ are selected from hydroxy; halogen; amino; C(═NH)NHR⁹; C(═O)NR¹⁰R¹¹; a non-aromatic monocyclic heterocyclic group of 3 to 6 ring members, of which 1 or 2 are heteroatom ring members selected from O and N, the carbocyclic or heterocyclic group being optionally substituted with 1 or 2 substituents R^{7d}; and an aromatic heterocyclic group selected from pyrrole, imidazole, pyrazole, indole and pyridone, the aromatic heterocyclic group being optionally substituted with 1 or 2 substituents R^{7e}.

1.28 A compound according to Embodiment 1.27 wherein the optional substituents R⁸ are selected from hydroxy; fluorine; amino; C(═O)NR¹⁰R¹¹; a non-aromatic monocyclic carbocyclic or heterocyclic group of 3 to 6 ring members, of which 0, 1 or 2 are heteroatom ring members selected from N, the heterocyclic group being optionally substituted with 1 or 2 substituents R^{7d}; and an aromatic heterocyclic group selected from pyrrole, imidazole, pyrazole, indole and pyridone, the aromatic heterocyclic group being optionally substituted with 1 or 2 substituents R^{7e}.

1.28A A compound according to Embodiment 1.27 or Embodiment 1.27A wherein the optional substituents R⁸ are selected from hydroxy; fluorine; amino; C(═O)NR¹⁰R¹¹; a non-aromatic monocyclic heterocyclic group of 3 to 6 ring members, of which 1 or 2 are heteroatom ring members selected from N, the heterocyclic group being optionally substituted with 1 or 2 substituents R^{7d}; and an aromatic heterocyclic group selected from pyrrole, imidazole, pyrazole, indole and pyridone, the aromatic heterocyclic group being optionally substituted with 1 or 2 substituents R^{7e}.

1.29 A compound according to Embodiment 1.28 wherein the optional substituents R⁸ are selected from hydroxy; amino; C(═O)NR¹⁰R¹¹; cyclopropyl; a non-aromatic monocyclic heterocyclic group of 5 to 6 ring members selected from piperidine and pyrrolidine; and an aromatic heterocyclic group selected from pyrrole and imidazole.

1.30 A compound according to Embodiment 1.29 wherein the optional substituents R⁸ are selected from hydroxy and C(═O)NR¹⁰R¹¹.

1.31 A compound according to Embodiment 1.30 wherein the optional substituents R⁸ are selected from C(═O)NR¹⁰R¹¹.

1.32 A compound according to any one of Embodiments 1.1 to 1.24 wherein R² is selected from hydrogen and R^{2a} wherein R^{2a} is selected from a C₁₋₈ alkyl group optionally substituted with one or two substituents R⁸; a monocyclic carbocyclic or heterocyclic group of 4 to 6 ring members selected from C₄₋₆ cycloalkyl, piperidine, imidazole, pyridine and tetrahydropyridine; and a bicyclic heterocyclic group selected from tetrahydroisoquinoline, tetrahydroquinoline, dihydroindole and dihydroisoindole; the monocyclic carbocyclic or heterocyclic group and the bicyclic heterocyclic group each being optionally substituted with one or two substituents R^{7b}; wherein the one or two substituents R⁸ are selected from hydroxy; amino; C(═NH)NHR⁹; C(═O)NR¹⁰R¹¹; a non-aromatic monocyclic carbocyclic or heterocyclic group of 3 to 6 ring members, of which 0, 1 or 2 are heteroatom ring members selected from N, the heterocyclic group being optionally substituted with 1 or 2 substituents R^{7d}; and an aromatic heterocyclic group selected from pyrrole, imidazole, pyrazole, indole and pyridone, the aromatic heterocyclic group being optionally substituted with 1 or 2 substituents R^{7e}.

1.33 A compound according to Embodiment 1.32 wherein R² is selected from hydrogen and R^{2a} wherein R^{2a} is selected from a C₁₋₈ alkyl group optionally substituted with a substituent R⁸; a monocyclic carbocyclic or heterocyclic group of 5 or 6 ring members selected from C₄₋₆ cycloalkyl, piperidine, imidazole, pyridine; and a bicyclic heterocyclic group selected from tetrahydroisoquinoline and dihydroisoindole; the monocyclic carbocyclic or heterocyclic group and the bicyclic heterocyclic group each being optionally substituted with one or two substituents R^{7b};

wherein the substituent R⁸ is selected from hydroxy; amino; C(═O)NR¹⁰R¹¹; cyclopropyl; piperidine and pyrrolidine; and an aromatic heterocyclic group selected from pyrrole, imidazole, pyrazole, indole and pyridone, the aromatic heterocyclic group being optionally substituted with 1 or 2 substituents R^{7e}.

1.34 A compound according to Embodiment 1.33 wherein R² is selected from hydrogen and R^{2a} wherein R^{2a} is selected from a C₁₋₈ alkyl group optionally substituted with a substituent R⁸; cyclohexyl substituted with a substituent R^{7b}; pyridine optionally substituted with a substituent R^{7b}; and tetrahydroisoquinoline; wherein the substituent R⁸ is selected from hydroxy; C(═O)NR¹⁰R¹¹; piperidine; pyrrole and imidazole.

1.35 A compound according to Embodiment 1.34 wherein R² is selected from hydrogen and a group R^{2a} wherein R^{2a} is a C₁₋₈ alkyl group optionally substituted with a substituent R⁸; wherein the substituent R⁸ is selected from hydroxy; C(═O)NR¹⁰R¹¹; piperidine; pyrrole and imidazole.

1.36 A compound according to Embodiment 1.35 wherein R² is selected from hydrogen and a group R^{2a} wherein R^{2a} is an C₁₋₈ alkyl group optionally substituted with a substituent R⁸; wherein the substituent R⁸ is selected from hydroxy and C(═O)NR¹⁰R¹¹.

1.37 A compound according to Embodiment 1.35 wherein R² is hydrogen.

1.38 A compound according to any one of Embodiments 1.1 to 1.36 wherein R² is a group R^{2a}.

1.39 A compound according to Embodiment 1.38 wherein R^{2a} is a C₁₋₈ alkyl group optionally substituted with a substituent R⁸; wherein the substituent R⁸ is selected from hydroxy and C(═O)NR¹⁰R¹¹.

1.39A A compound according to Embodiment 1.38 wherein R^{2a} is a C₁₋₈ alkyl group substituted with a substituent R⁸; wherein the substituent R⁸ is selected from hydroxy and C(═O)NR¹⁰R¹¹.

1.40 A compound according to Embodiment 1.38 wherein R^{2a} is a C₁₋₈ alkyl group substituted with a substituent R⁸; wherein the substituent R⁸ is selected from hydroxy and C(═O)NR¹⁰R¹¹.

1.41 A compound according to Embodiment 1.38 wherein R^{2a} is a C₁₋₈ alkyl group substituted with a substituent R⁸ which is C(═O)NR¹⁰R¹¹.

1.42 A compound according to any one of Embodiments 1.38 to 1.41 wherein, when R^{2a} is a optionally substituted C₁₋₈ alkyl group, it is selected from —CH₂CH₂-Opt, —CH(Alk)CH₂-Opt, —CH₂CH₂CH₂-Opt and —CH(Alk)CH₂CH₂-Opt where Opt is a hydrogen atom or the optional substituent, and Alk is methyl, ethyl or isopropyl.

1.43 A compound according to Embodiment 1.42 wherein, when R^{2a} is an optionally substituted C₁₋₈ alkyl group, it is selected from —CH₂CH₂-Opt and —CH(Alk)CH₂-Opt, where Opt is a hydrogen atom or the optional substituent, and Alk is methyl, ethyl or isopropyl.

1.44 A compound according to either of Embodiments 1.42 and 1.43 wherein Alk is methyl.

1.45 A compound according to Embodiment 1.43 or Embodiment 1.44 wherein R^{2a} is —*CH(Alk)CH₂-Opt and the asterisk denotes a chiral centre which is in the R-configuration.

1.45A A compound according to Embodiment 1.43 or Embodiment 1.44 wherein R^{2a} is —*CH(Alk)CH₂-Opt and the asterisk denotes a chiral centre which is in the S-configuration.

1.46 A compound according to any one of Embodiments 1.1 to 1.36 and 1.38 to 1.45 wherein R¹⁰ is selected from hydrogen and C₁₋₂ alkyl.

1.47 A compound according to Embodiment 1.46 wherein R¹⁰ is hydrogen.

1.48 A compound according to any one of Embodiments 1.1 to 1.36 and 1.38 to 1.41 wherein NR¹⁰R¹¹ forms a non-aromatic heterocyclic ring having a total of 4 to 7 ring members of which 1 or 2 are nitrogen atoms and the others are carbon atoms, the said non-aromatic heterocyclic ring being optionally substituted with one or more substituents selected from hydroxy, amino and C₁₋₄ alkyl.

1.49 A compound according to any one of Embodiments 1.1 to 1.36 and 1.38 to 1.47 wherein R¹¹ is selected from hydrogen; hydroxy; C₁₋₄ alkoxy; amino; mono- or di-C₁₋₄ alkylamino; a monocyclic non-aromatic carbocyclic or heterocyclic group of 3 to 7 ring members, of which 0, 1 or 2 are heteroatom ring members selected from O, N and S, the non-aromatic carbocyclic or heterocyclic group being optionally substituted with one or two substituents R^{7f}; unsubstituted C₁₋₂ alkyl and C₁₋₆ alkyl substituted with 1, 2 or 3 substituents R¹².

1.49A A compound according to Embodiment 1.49 wherein the substituted C₁₋₆ alkyl is an unbranched (straight chain) alkyl group.

1.49B A compound according to any one of Embodiments 1.1 to 1.36 and 1.38 to 1.47 wherein R¹¹ is selected from hydrogen; hydroxy; methoxy; amino; mono- or di-C₁₋₄ alkylamino; a monocyclic non-aromatic carbocyclic or heterocyclic group of 3 to 7 ring members, of which 0, 1 or 2 are heteroatom ring members selected from O and N, the non-aromatic heterocyclic group being optionally substituted with one or two substituents R^{7f}; and C₁₋₆ alkyl, wherein the C₁₋₆ alkyl is optionally substituted with 1, 2 or 3 substituents R¹².

1.49C A compound according to Embodiment 1.49B wherein the optionally substituted C₁₋₆ alkyl is an unbranched (straight chain) alkyl group.

1.50 A compound according to Embodiment 1.49 or Embodiment 1.49A wherein R¹¹ is selected from hydrogen; amino; a monocyclic non-aromatic heterocyclic group of 3 to 7 ring members, of which 1 or 2 are heteroatom ring members each of which is selected from O and N; unsubstituted C₁₋₆ alkyl; and C₁₋₆ alkyl substituted with 1, 2 or 3 substituents R¹².

1.50A A compound according to Embodiment 1.50 wherein the unsubstituted C₁₋₆ alkyl and the substituted C₁₋₆ alkyl are each an unbranched (straight chain) alkyl group.

1.51 A compound according to any one of Embodiments 1.1 to 1.36, 1.38 to 1.47 and 1.49 to 1.50A wherein the substituted C₁₋₆ alkyl is substituted with a single substituent R¹².

1.52 A compound according to any one of Embodiments 1.1 to 1.36, 1.38 to 1.47 and 1.49 to 1.51 wherein R¹² is selected from hydroxy; C₁₋₄ alkoxy; cyano; C₁₋₄alkoxycarbonyl; C₃₋₆cycloalkylamino; CONH₂; CONH(C₁₋₄alkyl); CON(C₁₋₄alkyl)₂ and a group —NH—CH₂-Cyc; where Cyc is a benzene, furan, thiophene or pyridine ring.

1.52A A compound according to any one of Embodiments 1.1 to 1.36, 1.38 to 1.47 and 1.49 to 1.51 wherein R¹² is selected from hydroxy; cyano; amino; mono- or di-C₁₋₄ alkylamino; CONH₂; and a group —NH—Bn; where Bn is a benzyl group.

1.52B A compound according to any one of Embodiments 1.1 to 1.36, 1.38 to 1.47 and 1.49 to 1.51 wherein R¹² is selected from hydroxy; cyano; CONH₂; and a group —NH—Bn; where Bn is a benzyl group.

1.53 A compound according to Embodiment 1.49 wherein R¹¹ is selected from:
hydrogen;
hydroxy;
methoxy;
amino;
mono- or di-C₁₋₄ alkylamino;
a monocyclic non-aromatic heterocyclic group of 3 to 7 ring members, of which 1 or 2 are heteroatom ring members selected from O and N provided that at least one heteroatom ring member is nitrogen, the non-aromatic heterocyclic group being optionally substituted with one or two substituents R^{7f}; and
unsubstituted C₁₋₂ alkyl;
C₁₋₆ alkyl substituted with a substituent R¹² selected from hydroxy; cyano; CONH₂; and a group —NH—CH₂-Cyc; where Cyc is a benzene ring.

1.53A A compound according to Embodiment 1.49 wherein R¹¹ is selected from:
hydrogen;
hydroxy;
methoxy;
amino;
mono- or di-C₁₋₄ alkylamino;
a monocyclic non-aromatic heterocyclic group of 3 to 7 ring members, of which 1 or 2 are heteroatom ring members selected from O and N provided that at least one heteroatom ring member is nitrogen, the non-aromatic heterocyclic group being optionally substituted with one or two substituents R^{7f}; and
unsubstituted C₁₋₂ alkyl; and
C₁₋₆ alkyl substituted with a substituent R¹² selected from hydroxy; amino; cyano; CONH₂; and a group —NH—CH₂-Cyc; where Cyc is a benzene ring.

1.54 A compound according to Embodiment 1.53 wherein R¹¹ is selected from:
hydrogen;
hydroxy;
methoxy;
amino;
mono- or di-C₁₋₄ alkylamino;
a monocyclic non-aromatic heterocyclic group of 3 to 7 ring members, of which 1 or 2 are heteroatom ring members selected from O and N provided that at least one heteroatom ring member is nitrogen, the non-aromatic heterocyclic group being optionally substituted with one or two substituents R^{7f}; and
unsubstituted C₁₋₂ alkyl;
C₁₋₄ alkyl substituted with a substituent R¹² selected from hydroxy; cyano; CONH₂; and a group —NH—CH₂-Cyc; where Cyc is a benzene ring.

1.54A A compound according to Embodiment 1.53A wherein R¹¹ is selected from:
hydrogen;
hydroxy;
methoxy;
amino;
mono- or di-C₁₋₄ alkylamino;
a monocyclic non-aromatic heterocyclic group of 3 to 7 ring members, of which 1 or 2 are heteroatom ring members selected from O and N provided that at least one heteroatom ring member is nitrogen, the non-aromatic heterocyclic group being optionally substituted with one or two substituents R^{7f}; and
unsubstituted C₁₋₂ alkyl;
C₁₋₄ alkyl substituted with a substituent R¹² selected from hydroxy; amino; cyano; CONH₂; and a group —NH—CH₂-Cyc; where Cyc is a benzene ring.

1.54B A compound according to Embodiment 1.54A wherein R¹¹ is selected from hydrogen and amino-C₂₋₃alkyl.

1.54C A compound according to Embodiment 1.54A wherein R¹¹ is selected from hydrogen and 2-aminoethyl.

1.55 A compound according to Embodiment 1.54 wherein R¹¹ is hydrogen.

1.55 A compound according to Embodiment 1.54 wherein R¹¹ is 2-aminoethyl.

1.56 A compound according to any one of Embodiments 1.1 to 1.55 wherein R^{7a} is selected from amino; hydroxy; C₁₋₄ alkyl; hydroxy-C₁₋₃ alkyl; and amino-C₁₋₃ alkyl.

1.56A A compound according to Embodiment 1.56 wherein R^{7a} is selected from amino; hydroxy; hydroxymethyl; aminomethyl and methyl.

1.56B A compound according to any one of Embodiments 1.1 to 1.55 wherein R^{7a} is absent.

1.56C A compound according to any one of Embodiments 1.1 to 1.56B wherein R^{7b} is selected from amino; hydroxy; C₁₋₄ alkyl; hydroxy-C₁₋₃ alkyl; and amino-C₁₋₃ alkyl.

1.56D A compound according to Embodiment 1.56C wherein R^{7b} is selected from amino; hydroxy; hydroxymethyl; aminomethyl and methyl.

1.56E A compound according to any one of Embodiments 1.1 to 1.56B wherein R^{7b} is absent.

1.56F A compound according to any one of Embodiments 1.1 to 1.55 wherein R^{7c} is selected from amino; hydroxy; C₁₋₄ alkyl; hydroxy-C₁₋₃ alkyl; and amino-C₁₋₃ alkyl.

1.56G A compound according to Embodiment 1.56F wherein R^{7c} is selected from amino; hydroxy; hydroxymethyl; aminomethyl and methyl.

1.56H A compound according to any one of Embodiments 1.1 to 1.56E wherein R^{7c} is absent.

1.56J A compound according to any one of Embodiments 1.1 to 1.56H wherein R^{7d} is selected from amino; hydroxy; C₁₋₄ alkyl; hydroxy-C₁₋₃ alkyl; and amino-C₁₋₃ alkyl.

1.56K A compound according to Embodiment 1.56J wherein R^{7d} is selected from amino; hydroxy; hydroxymethyl; aminomethyl and methyl.

1.56L A compound according to any one of Embodiments 1.1 to 1.56H wherein R^{7c} is absent.

1.56M A compound according to any one of Embodiments 1.1 to 1.56L wherein R^{7e} is selected from amino; hydroxy; C₁₋₄ alkyl; hydroxy-C₁₋₃ alkyl; and amino-C₁₋₃ alkyl.

1.56N A compound according to Embodiment 1.56M wherein R^{7e} is selected from methyl and ethyl.

1.56P A compound according to any one of Embodiments 1.1 to 1.56L wherein R^{7e} is absent.

1.56Q A compound according to any one of Embodiments 1.1 to 1.56P wherein R^{7f} is selected from amino; hydroxy; C₁₋₄ alkyl; hydroxy-C₁₋₃ alkyl; and amino-C₁₋₃ alkyl.

1.56R A compound according to Embodiment 1.56Q wherein R^{7f} is selected from amino; hydroxy; hydroxymethyl; aminomethyl and methyl.

1.56S A compound according to Embodiment 1.56R wherein R^{7f} is hydroxymethyl.

1.56T A compound according to any one of Embodiments 1.1 to 1.56P wherein R^{7f} is absent.

1.57 A compound according to any one of Embodiments 1.1 to 1.56T wherein R⁴ is selected from hydrogen and a substituent R^{4a}; wherein R^{4a} is selected from fluorine, chlorine, cyano; C₁₋₂ alkyl optionally substituted with one or more fluorine atoms; C₁₋₂ alkoxy optionally substituted with one or more fluorine atoms; hydroxy-C₁₋₂ alkyl; and C₁₋₂ alkoxy-C₁₋₂ alkyl.

1.57A A compound according to Embodiment 1.57 wherein R^{4a} is selected from fluorine, chlorine, cyano; methyl, ethyl, difluoromethyl, trifluoromethyl, methoxy, trifluoromethoxy, difluoromethoxy, hydroxymethyl, hydroxyethyl, methoxymethyl and methoxyethyl.

1.57B A compound according to Embodiment 1.57A wherein R^{4a} is selected from fluorine, chlorine, cyano; methyl, ethyl, difluoromethyl, trifluoromethyl and methoxy.

1.57C A compound according to Embodiment 1.57B wherein R^{4a} is selected from fluorine, chlorine and methyl.

1.57D A compound according to Embodiment 1.57C wherein R^{4a} is selected from fluorine and chlorine.

1.57E A compound according to Embodiment 1.57D wherein R^{4a} is fluorine.

1.57F A compound according to Embodiment 1.57D wherein R^{4a} is chlorine.

1.57G A compound according to any one of Embodiments 1.1 to 1.57F wherein R⁴ is a substituent R^{4a}.

1.57H A compound according to any one of Embodiments 1.1 to 1.57 wherein R⁴ is hydrogen.

1.58 A compound according to any one of Embodiments 1.1 to 1.57H wherein R⁵ is selected from hydrogen and a substituent R^{5a}; and R^{5a} is selected from fluorine, chlorine, cyano, C₁₋₂ alkyl optionally substituted with one or more fluorine atoms; C₁₋₂ alkoxy optionally substituted with one or more fluorine atoms; cyclopropyl; and amino.

1.58A A compound according to Embodiment 1.58 wherein R^{5a} is selected from fluorine, chlorine, cyano, methyl, ethyl, difluoromethyl, trifluoromethyl, methoxy, trifluoromethoxy and difluoromethoxy.

1.58B A compound according to Embodiment 1.58A wherein R^{5a} is selected from fluorine, chlorine, methyl and ethyl.

1.58C A compound according to Embodiment 1.58B wherein R^{5a} is fluorine or chlorine.

1.58D A compound according to Embodiment 1.58C wherein R^{5a} is chlorine.

1.58E A compound according to Embodiment 1.58C wherein R⁵ is fluorine.

1.58F A compound according to any one of Embodiments 1.1 to 1.58E wherein R⁵ is a substituent R^{5a}.

1.58G A compound according to any one of Embodiments 1.1 to 1.58 wherein R⁵ is hydrogen.

1.59 A compound according to any one of Embodiments 1.1 to 1.58G wherein R³ is selected from 6-membered monocyclic aryl and heteroaryl groups containing 0, 1 or 2 nitrogen ring members and being optionally substituted with one or more substituents R¹³; 9-membered bicyclic heteroaryl groups containing 1, 2, 3 or 4 heteroatom ring members selected from O, N and S and being optionally substituted with one or more substituents R¹³; 9- and 10-membered partially aromatic bicyclic heterocyclic groups containing a benzene ring fused to a non-aromatic 5- or 6-membered heterocyclic ring containing 1 or 2 heteroatoms selected from O, N and S, the said partially aromatic bicyclic heterocyclic groups being optionally substituted with one or more substituents selected from oxo and R¹³.

1.59A A compound according to any one of Embodiments 1.1 to 1.59 wherein R³ is selected from phenyl and pyridyl, each being optionally substituted with one or more substituents R¹³; and 9-membered partially aromatic bicyclic heterocyclic groups containing a benzene ring fused to a non-aromatic 5-membered heterocyclic ring containing 1 or 2 heteroatoms selected from O and N, the said partially aromatic bicyclic heterocyclic groups being optionally substituted with one or more substituents R¹³.

1.60 A compound according to Embodiment 1.59A wherein R³ is selected from phenyl and pyridyl, each being optionally substituted with one or more substituents R¹³; and 9-membered partially aromatic bicyclic heterocyclic groups containing a benzene ring fused to a non-aromatic 5-membered heterocyclic ring containing 1 or 2 heteroatoms selected from O and N, the said partially aromatic bicyclic heterocyclic groups being unsubstituted or being substituted with one or two substituents selected from C₁₋₄ alkyl.

1.61 A compound according to Embodiment 1.60 wherein R³ is selected from phenyl and pyridyl, each being optionally substituted with one or more substituents R¹³.

1.62 A compound according to Embodiment 1.61 wherein R³ is selected from phenyl optionally substituted with one or more substituents R¹³.

1.63 A compound according to Embodiment 1.61 wherein R³ is selected from pyridyl optionally substituted with one or more substituents R¹³.

1.63A A compound according to any one of Embodiments 1.1 to 1.61 and 1.63 wherein R³ is other than a substituted or unsubstituted pyridone or pyrimidone group.

1.64 A compound according to Embodiment 1.59 wherein R³ is a 9-membered partially aromatic bicyclic heterocyclic group containing a benzene ring fused to a non-aromatic 5-membered heterocyclic ring containing 1 or 2 heteroatoms selected from O and N, the said partially aromatic bicyclic heterocyclic groups being the said partially aromatic bicyclic heterocyclic groups being optionally substituted with one or more substituents R¹³.

1.65 A compound according to Embodiment 1.64 wherein the partially aromatic bicyclic heterocyclic groups being unsubstituted or is substituted with 1 or 2 methyl substituents.

1.66 A compound according to any one of Embodiments 1.1 to 1.64 wherein the substituents R¹³ are selected from halogen; cyano; nitro; CH═NOH; and a group R^{a}-R^{b}; and are optionally further selected from oxo;
R^{a} is a bond, O, CO, X¹C(X²), C(X²)X¹, SO₂, NR^{c}, SO₂NR^{c} or NR^{c}SO₂;
R^{b} is hydrogen; a cyclic group R^{d}; or an acyclic C₁₋₈ hydrocarbon group optionally substituted with one or more substituents selected from hydroxy, oxo, halogen, cyano, amino, mono- or di-C₁₋₄ alkylamino, and a cyclic group R^{d}; wherein one or two but not all of the carbon atoms of the acyclic C₁₋₈ hydrocarbon group may optionally be replaced by O, NR^{c}, X¹C(X²), C(X²)X¹ or X¹C(X²)X¹; SO₂NR^{c} or NR^{c}SO₂, but excluding the combination wherein R^{a} is a bond and R^{b} is hydrogen;
the cyclic group R^{d} is a monocyclic carbocyclic or heterocyclic group having from 3 to 7 ring members, of which 0, 1, 2 or 3 are heteroatom ring members selected from O and N, the carbocyclic or heterocyclic group being optionally substituted with one or more substituents selected from R¹⁴;
R¹⁴ is selected from cyano; and R^{a}-R^{e};
R^{e} is hydrogen or an acyclic C₁₋₈ hydrocarbon group optionally substituted with one or more substituents selected from phenyl and hydroxy
X¹ is O or NR^{c};
X² is ═O or ═NR^{c}; and
R^{c} is hydrogen or C₁₋₄ alkyl.

1.67 A compound according to Embodiment 1.66 wherein the substituents R¹³ are selected from halogen; cyano; nitro; CH═NOH; and a group R^{a}-R^{b}; and are optionally further selected from oxo;
R^{a} is a bond, O, CO, X¹C(X²), C(X²)X¹, NR^{c}, SO₂NR^{c} or NR^{c}SO₂;
R^{b} is hydrogen; a cyclic group R^{d}; or an acyclic C₁₋₈ hydrocarbon group optionally substituted with one or more substituents selected from hydroxy, halogen, cyano, and a cyclic group R^{d}; wherein one or two but not all of the carbon atoms of the acyclic C₁₋₈ hydrocarbon group may optionally be replaced by O, NR^{c}, SO₂NR^{c} or NR^{c}SO₂, but excluding the combination wherein R^{a} is a bond and R^{b} is hydrogen;
the cyclic group R^{d} is a monocyclic heterocyclic group having from 3 to 7 ring members, of which 1 or 2 are heteroatom ring members selected from O, N and S and oxidised forms thereof, the carbocyclic or heterocyclic group being optionally substituted with one or more substituents selected from R¹⁴; and
R¹⁴ is R^{a}-R^{e}; and R^{e} is an acyclic C₁₋₈ hydrocarbon group substituted with phenyl.

1.68 A compound according to any one of Embodiments 1.1 to 1.67 wherein either no substituents R¹³ are present or 1, 2 or 3 substituents R¹³ are present and are selected from halogen; cyano; nitro; CH═NOH; and a group R^{a}-R^{b}; and are optionally further selected from oxo; wherein
R^{a} is a bond, O, CO, X¹C(X²), C(X²)X¹, NR^{c}, SO₂NR^{c} or NR^{c}SO₂;
R^{b} is hydrogen; a cyclic group R^{d}; or an acyclic C₁₋₈ hydrocarbon group optionally substituted with one or more substituents selected from hydroxy, halogen, cyano, and a cyclic group R^{d}; wherein one or two but not all of the carbon atoms of the acyclic C₁₋₈ hydrocarbon group may optionally be replaced by O, NR^{c}, SO₂NR^{c} or NR^{c}SO₂, but excluding the combination wherein R^{a} is a bond and R^{b} is hydrogen;
the cyclic group R^{d} is a monocyclic heterocyclic group having from 3 to 7 ring members, of which 1 or 2 are heteroatom ring members selected from O, N and S and oxidised forms thereof, the carbocyclic or heterocyclic group being optionally substituted with one or more substituents selected from R¹⁴; and
R¹⁴ is R^{a}-R^{e}; and R^{e} is an acyclic C₁₋₈ hydrocarbon group substituted with phenyl.

1.69 A compound according to Embodiment 1.68 wherein either no substituents R¹³ are present or 1, 2 or 3 substituents R¹³ are present and are selected from fluorine; chlorine; cyano; nitro; CH═NOH; and a group R^{a}-R^{b}; and are optionally further selected from oxo; wherein
R^{a} is a bond, O, CO, CONR^{c}, NR^{c}CO, NR^{c}, SO₂NR^{c} or NR^{c}SO₂;
R^{b} is hydrogen; a cyclic group R^{d}; or a C₁₋₈ alkyl group optionally substituted with one or more substituents selected from hydroxy, fluorine, cyano, and a cyclic group R^{d}; wherein one or two but not all of the carbon atoms of the acyclic C₁₋₈ hydrocarbon group may optionally be replaced by O, NR^{c}, SO₂NR^{c} or NR^{c}SO₂;
the cyclic group R^{d} is a monocyclic heterocyclic group having from 3 to 7 ring members, of which 1 or 2 are heteroatom ring members selected from O, N and S and oxidised forms thereof, the heterocyclic group being optionally substituted with one or more substituents selected from R¹⁴; and
R¹⁴ is R^{a}-R^{e}; and R^{e} is benzyl.

1.70 A compound according to Embodiment 1.69 wherein either no substituents R¹³ are present or 1, 2 or 3 substituents R¹³ are present and are selected from fluorine; chlorine; cyano; nitro; CH═NOH; and a group R^{a}-R^{b}; and are optionally further selected from oxo; wherein
R^{a} is a bond, O, CO, CONR^{c}, NR^{c}CO, NR^{c}, SO₂NR^{c} or NR^{c}SO₂;
R^{b} is a cyclic group R^{d}; C₂₋₃ alkynyl; or a C₁₋₈ alkyl group optionally substituted with one or more substituents selected from hydroxy, fluorine, cyano, and a cyclic group R^{d}; wherein one or two but not all of the carbon atoms of the C₁₋₆ alkyl group may optionally be replaced by NR^{c}SO₂ and wherein the cyclic group R^{d} is a monocyclic heterocyclic group having from 4-6 ring members, of which 1 or 2 are heteroatom ring members selected from O and N, the heterocyclic group being optionally substituted with one or more substituents selected from R¹⁴; wherein R¹⁴ is R^{a}-R^{e}; and R^{e} is benzyl.

1.71 A compound according to any one of Embodiments 1.68 to 1.70 wherein either no substituents R¹³ are present or 1 or 2 substituents R¹³ are present.

1.72 A compound according to Embodiment 1.71 wherein no substituents R¹³ are present.

1.73 A compound according to Embodiment 1.71 wherein one substituent R¹³ is present.

1.74 A compound according to Embodiment 1.71 wherein two substituents R¹³ are present.

1.74A A compound according to any one of Embodiments 1.1 to 1.65 wherein either no substituents R¹³ are present or one or two substituents R¹³ are present and are selected from:
—(CH₂)_{y}NHSO₂CH₃ where y is 0 or 1;
C₁₋₂ alkyl optionally substituted with cyano, hydroxy or methoxyl or with one or more fluorine atoms;
C₁₋₂ alkoxy
pyrrolidinylcarbonyl;
C(O)NHR¹⁹; where R¹⁹ is hydrogen or C₁₋₂ alkyl optionally substituted with cyano;
C(O)NR²⁰R²¹ where R²⁰ is methyl and R²¹ is pyrazol-4-ylmethyl or 1-benzylpyrazol-4-ylmethyl;
—CH(CH₃)OC(O)NHCH₂CH₃;
CH₂OC(O)NHCH₂Cyp where Cyp is cyclopropyl;
halogen;
C(O)NH₂
acetylamino;
nitro;
cyano;
amino wherein the amino is optionally substituted with one or two C₁₋₂ alkyl groups;
C₁₋₂ alkylsulphonyl;
—NH(CO)NHCH₂CF₃;
—CH₂NHC(O)CH₃;
methyloxadiazolyl;
oxazolyl;
—SO₂NHCH₃;
cyclopropyl optionally substituted with cyano or hydroxymethyl;
CH═N—OH;
ethynyl.

1.74B A compound according to Embodiment 1.74A wherein either no substituents R¹³ are present or one or two substituents R¹³ are present and are selected from amino; hydroxy-C₁₋₃alkyl; C₁₋₄ alkyl; and halogen.

1.74C A compound according to Embodiment 1.74A wherein either no substituents R¹³ are present or one or two substituents R¹³ are present and are selected from amino; hydroxymethyl; methyl; and chlorine.

1.74D A compound according to Embodiment 1.74A wherein either no substituents R¹³ are present or one substituent R¹³ is present and is selected from amino and hydroxymethyl.

1.75 A compound according to any one of Embodiments 1.1 to 1.74 wherein when A and E are both CH, R⁰ is hydrogen, R⁴ and R⁵ are both hydrogen, R³ is phenyl and R¹ is hydrogen, then R² is other than 2-amino-pyridin-3-yl; 6-amino-pyridin-2-yl; 2-methyl-pyridin-4-yl; azetidin-3-yl; and 5-amino-pyridin-2-yl.

1.76 A compound according to any one of Embodiments 1.1 to 1.74 wherein when A and E are both CH, R⁰ is hydrogen, R⁴ and R⁵ are both fluorine, R³ is phenyl and R¹ is hydrogen, then R² is other than 6-amino-pyridin-2-yl and pyridin-2-yl.

1.77 A compound according to any one of Embodiments 1.1 to 1.74 wherein when A and E are both CH, R⁰ is hydrogen, R⁴ and R⁵ are both fluorine, R³ is 3-methanesulphonylamino-phenyl and R¹ is hydrogen, then R² is other than 2-methylimidazol-4-yl.

1.78 A compound according to any one of Embodiments 1.1 to 1.74 wherein when A and E are both CH, R⁰ is hydrogen, R⁴ and R⁵ are both hydrogen, R³ is pyridin-2-yl and R¹ is hydrogen, then R² is other than 4-aminocyclohexyl.

1.79 A compound according to any one of Embodiments 1.1 to 1.74 wherein when A and E are both CH, R⁰ is hydrogen, R¹ is hydrogen, R⁴ and R⁵ are both fluorine and R² is 5-methyl-pyridin-2-yl, then R³ is other than phenyl and 4-amino-3-methylphenyl.

1.80 A compound according to any one of Embodiments 1.1 to 1.74 wherein when A and E are both CH, R⁰ is hydrogen, R⁴ and R⁵ are both fluorine, R³ is phenyl and R² is hydrogen, then R¹ is other than nitromethyl; acetamidomethyl; cyano; and carbamoylmethyl.

1.81 A compound according to any one of Embodiments 1.1 to 1.74 wherein when A and E are both CH, R⁰ is hydrogen, R⁴ and R⁵ are both fluorine, R³ is phenyl and R¹ is ethyl, then R² is other than 2-pyridone-6-yl.

1.82 A compound according to any one of Embodiments 1.1 to 1.74 wherein when A and E are both CH, R⁰ is hydrogen, R⁴ and R⁵ are both fluorine, R³ is phenyl, R¹ is ethyl and the carbon atom to which R¹ is attached is in an S stereochemical configuration, then R² is other than 2-(N-succinimido)-ethyl.

1.83 A compound according to any one of Embodiments 1.1 to 1.74 wherein when A and E are both CH, R⁰ is hydrogen, R⁴ is fluorine, R⁵ is isopropyl, R³ is phenyl and R² is hydrogen, then R¹ is other than piperidin-4-ylmethyl.

1.84 A compound according to any one of Embodiments 1.1 to 1.74 wherein when A and E are both CH, R⁰ is hydrogen, R⁴ is fluorine and R⁵ is chlorine, R³ is phenyl, and R¹ is ethyl, then R² is other than 2-oxopiperidin-4-yl.

1.85 A compound according to any one of Embodiments 1.1 to 1.74 wherein when A and E are both CH, R⁰ is hydrogen, R⁴ is fluorine, R⁵ is chlorine, R³ is phenyl, R¹ is ethyl and the carbon atom to which R¹ is attached is in an R stereochemical configuration, then R² is other than a (pyrazol-4-yl)-CH(CH₃)— group; or a (morpholin-4-yl)-C(═O)—CH₂CH(CH₃)— group; or a (5-methyl-pyrazol-4-yl)-CH(CH₃)— group; or a CH₃O—CH₂CH₂—NH—C(═O)—CH₂CH(CH₃)— group; or an HOCH(CH₃)CH₂—NH—C(═O)—CH₂CH(CH₃)— group.

1.85A A compound according to any one of Embodiments 1.1 to 1.74 wherein when A and E are both CH, R⁰ is hydrogen, R⁴ is fluorine, R⁵ is chlorine, R³ is phenyl, R¹ is ethyl and the carbon atom to which R¹ is attached is in an S stereochemical configuration, then R² is other than a (pyrazol-4-yl)-CH(CH₃)— group; or a (morpholin-4-yl)-C(═O)—CH₂CH(CH₃)— group; or a (5-methyl-pyrazol-4-yl)-CH(CH₃)— group; or a CH₃O—CH₂CH₂—NH—C(═O)—CH₂CH(CH₃)— group; or an HOCH(CH₃)CH₂—NH—C(═O)—CH₂CH(CH₃)— group.

1.86 A compound according to any one of Embodiments 1.1 to 1.85 having the isomeric form (1a):

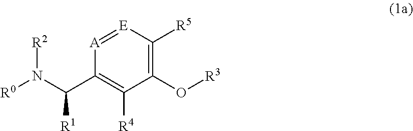

or a salt, N-oxide or tautomer thereof, wherein A, E, R⁰, R¹, R², R³, R⁴ and R⁵ are as defined in any one of Embodiments 1.1 to 1.85.

1.87 A compound according to any one of Embodiments 1.1 to 1.85 having the isomeric form (1b):

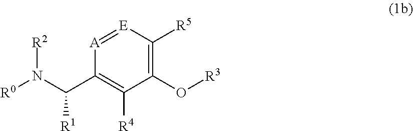

or a salt, N-oxide or tautomer thereof, wherein A, E, R⁰, R¹, R², R³, R⁴ and R⁵ are as defined in any one of Embodiments 1.1 to 1.85.

1.88 A compound according to Embodiment 1.86 having the formula (2):

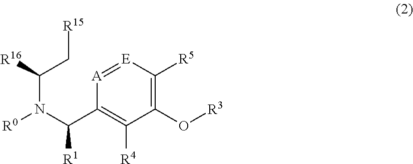

or a salt, N-oxide or tautomer thereof, wherein:

R¹⁵ is selected from hydrogen; a substituent R⁸; an acyclic C₁₋₃ hydrocarbon group optionally substituted with one or two substituents R⁸ wherein one carbon atom of the acyclic C₁₋₃ hydrocarbon group may optionally be replaced by a heteroatom or group selected from O and NR^{c} provided that at least one carbon atom of the acyclic C₁₋₃ hydrocarbon group remains; a monocyclic carbocyclic or heterocyclic group of 3 to 7 ring members, of which 0, 1 or 2 ring members are heteroatom ring members selected from O, N and S; and a bicyclic heterocyclic group of 9 or 10 ring members, of which 1 or 2 ring members are nitrogen atoms, one of the rings of the bicyclic heterocyclic group being a non-aromatic nitrogen-containing ring; the monocyclic carbocyclic or heterocyclic group and the bicyclic heterocyclic group each being optionally substituted with one or two substituents R^{7b};

R¹⁶ is selected from hydrogen and C₁₋₄ alkyl; and

A, E, R⁰, R¹, R³, R⁴, R⁵ and R⁸ are as defined in any one of Embodiments 1.1 to 1.85;

wherein at least one of R¹ and R² is other than hydrogen.

1.88A A compound according to Embodiment 1.188 having the formula (2a):

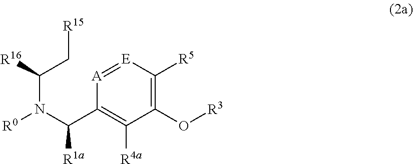

or a salt, N-oxide or tautomer thereof, wherein A, E, R⁰, R^{1a}, R³, R^{4a} and R⁵ are as defined in any one of Embodiments 1.1 to 1.56G and 1.57 to 1.88, and R¹⁵ and R¹⁶ are as defined in Embodiment 1.88.

1.89 A compound according to Embodiment 1.86 having the formula (3):

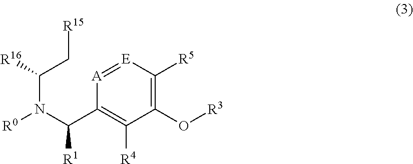

or a salt, N-oxide or tautomer thereof, wherein:

R¹⁵ is selected from hydrogen; a substituent R⁸; an acyclic C₁₋₃ hydrocarbon group optionally substituted with one or two substituents R⁸ wherein one carbon atom of the acyclic C₁₋₃ hydrocarbon group may optionally be replaced by a heteroatom or group selected from O and NR^{c} provided that at least one carbon atom of the acyclic C₁₋₃ hydrocarbon group remains; a monocyclic carbocyclic or heterocyclic group of 3 to 7 ring members, of which 0, 1 or 2 ring members are heteroatom ring members selected from O, N and S; and a bicyclic heterocyclic group of 9 or 10 ring members, of which 1 or 2 ring members are nitrogen atoms, one of the rings of the bicyclic heterocyclic group being a non-aromatic nitrogen-containing ring; the monocyclic carbocyclic or heterocyclic group and the bicyclic heterocyclic group each being optionally substituted with one or two substituents R^{7b};

R¹⁶ is selected from hydrogen and C₁₋₄ alkyl; and

A, E, R⁰, R¹, R³, R⁴, R⁵ and R⁸ are as defined in any one of Embodiments 1.1 to 1.85;

wherein at least one of R¹ and R² is other than hydrogen.

1.90 A compound according to Embodiment 1.87 having the formula (4):

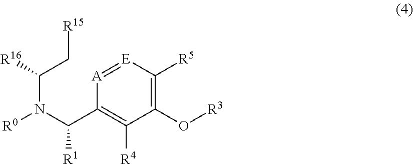

or a salt, N-oxide or tautomer thereof, wherein:

R¹⁵ is selected from hydrogen; a substituent R⁸; an acyclic C₁₋₃ hydrocarbon group optionally substituted with one or two substituents R⁸ wherein one carbon atom of the acyclic C₁₋₃ hydrocarbon group may optionally be replaced by a heteroatom or group selected from O and NR^{c} provided that at least one carbon atom of the acyclic C₁₋₃ hydrocarbon group remains; a monocyclic carbocyclic or heterocyclic group of 3 to 7 ring members, of which 0, 1 or 2 ring members are heteroatom ring members selected from O, N and S; and a bicyclic heterocyclic group of 9 or 10 ring members, of which 1 or 2 ring members are nitrogen atoms, one of the rings of the bicyclic heterocyclic group being a non-aromatic nitrogen-containing ring; the monocyclic carbocyclic or heterocyclic group and the bicyclic heterocyclic group each being optionally substituted with one or two substituents R^{7b};

R¹⁶ is selected from hydrogen and C₁₋₄ alkyl; and

A, E, R⁰, R¹, R³, R⁴, R⁵ and R⁸ are as defined in any one of Embodiments 1.1 to 1.85;

wherein at least one of R¹ and R² is other than hydrogen.

1.91 A compound according to Embodiment 1.87 having the formula (5):

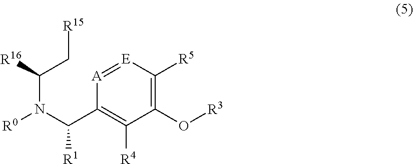

or a salt, N-oxide or tautomer thereof, wherein:

R¹⁵ is selected from hydrogen; a substituent R⁸; an acyclic C₁₋₃ hydrocarbon group optionally substituted with one or two substituents R⁸ wherein one carbon atom of the acyclic C₁₋₃ hydrocarbon group may optionally be replaced by a heteroatom or group selected from O and NR^{c} provided that at least one carbon atom of the acyclic C₁₋₃ hydrocarbon group remains; a monocyclic carbocyclic or heterocyclic group of 3 to 7 ring members, of which 0, 1 or 2 ring members are heteroatom ring members selected from O, N and S; and a bicyclic heterocyclic group of 9 or 10 ring members, of which 1 or 2 ring members are nitrogen atoms, one of the rings of the bicyclic heterocyclic group being a non-aromatic nitrogen-containing ring; the monocyclic carbocyclic or heterocyclic group and the bicyclic heterocyclic group each being optionally substituted with one or two substituents R^{7b};

R¹⁶ is selected from hydrogen and C₁₋₄ alkyl; and

A, E, R⁰, R¹, R³, R⁴, R⁵ and R⁸ are as defined in any one of Embodiments 1.1 to 1.85;

wherein at least one of R¹ and R² is other than hydrogen.

1.92 A compound according to any one of Embodiments 1.88 to 1.91A wherein R¹⁶ is C₁₋₃ alkyl.

1.93 A compound according to Embodiment 1.92 wherein R¹⁶ is methyl.

1.94 A compound according to any one of Embodiments 1.88 to 1.93 wherein R¹⁵ is selected from hydrogen; R⁸ and C₁₋₃ alkyl optionally substituted with a substituent R⁸.

1.94A A compound according to Embodiment 1.94 wherein R¹⁵ is selected from R⁸ and C₁₋₂ alkyl substituted with a substituent R⁸.

1.94B A compound according to Embodiment 1.94A wherein R¹⁵ is selected from hydrogen and C₁₋₃ alkyl.

1.94C A compound according to Embodiment 1.94A wherein R¹⁵ is selected from R⁸ wherein R⁸ is C(═O)NR¹⁰R¹¹.

1.94D A compound according to Embodiment 1.94C wherein R¹⁰ is hydrogen.

1.94E A compound according to Embodiment 1.94C or Embodiment 1.94D wherein R¹¹ is selected from hydrogen and hydroxy-C₁₋₄alkyl.

1.94F A compound according to Embodiment 1.94E wherein R¹¹ is hydrogen.

1.94G A compound according to Embodiment 1.94C or Embodiment 1.94D wherein R¹¹ is selected from hydrogen, amino-C₂₋₃alkyl and hydroxy-C₂₋₃alkyl.

1.94H A compound according to Embodiment 1.94G wherein R¹¹ is selected from hydrogen and 2-aminoethyl.

1.95 A compound according to Embodiment 1.88 wherein:

A is CH:

E is CH;

R⁰ is hydrogen;

R¹ is selected from C₁₋₆ alkyl (e.g. C₁₋₄ alkyl), cyclopropyl, hydroxy-C₁₋₄ alkyl and methoxy-C₁₋₃ alkyl;

R¹⁶ is selected from methyl and ethyl;

R¹⁵ is selected from C(O)NH₂ and C(O)NH(CH₂)₂OH;

R⁴ is fluorine or chlorine;

R⁵ is fluorine or chlorine; and

R³ is as defined in any one of Embodiments 1.1 and 1.59 to 1.74D.

1.95A A compound according to Embodiment 1.88 wherein:

A is CH:

E is CH;

R⁰ is hydrogen;

R¹ is selected from C₁₋₆ alkyl (e.g. C₁₋₄ alkyl), cyclopropyl, hydroxy-C₁₋₄ alkyl and methoxy-C₁₋₃ alkyl;

R¹⁶ is selected from methyl and ethyl;

R¹⁵ is selected from C(O)NH₂ and C(O)NH(CH₂)₂OH;

R⁴ is fluorine or chlorine;

R⁵ is fluorine or chlorine; and

R³ is selected from:
phenyl optionally substituted with one or two substituents selected from fluorine, chlorine, cyano, amino, C₁₋₄alkylsulphonylamino, C₁₋₄ acylamino, C₁₋₄alkyl, C₁₋₄alkoxy and five membered monocyclic heteroaryl groups containing one or two heteroatom ring members selected from O, N and S;
pyridyl optionally substituted with amino or carbamoyl; and
dihydrobenzofuranyl.

1.95B A compound according to Embodiment 1.88 wherein:
A is CH:
E is CH;
R⁰ is hydrogen;
R¹ is selected from C₁₋₆ alkyl (e.g. C₁₋₄ alkyl), cyclopropyl, hydroxy-C₁₋₄ alkyl and methoxy-C₁₋₃ alkyl;
R¹⁶ is selected from methyl and ethyl;
R¹⁵ is selected from C(O)NH₂, C(O)NH(CH₂)₂OH and C(O)NH(CH₂)₂NH₂;
R⁴ is fluorine or chlorine;
R⁵ is fluorine or chlorine; and
R³ is as defined in any one of Embodiments 1.1 and 1.59 to 1.74D.

1.96 A compound according to Embodiment 1.95A wherein:

A is CH:

E is CH;

R⁰ is hydrogen;

R¹ is selected from methyl, ethyl, cyclopropyl, methoxyethyl and hydroxyethyl;

R¹⁶ is selected from methyl and ethyl;

R¹⁵ is selected from C(O)NH₂ and C(O)NH(CH₂)₂OH;

R⁴ is fluorine;

R⁵ is chlorine; and

R³ is selected from:
phenyl optionally substituted with one or two substituents selected from fluorine, chlorine, cyano, amino, mesylamino, acetylamino, methyl, methoxy, cyanomethyl and oxazolyl;
pyridyl optionally substituted with amino or carbamoyl; and
dihydrobenzofuranyl.

1.96A A compound according to Embodiment 1.95B wherein:

A is CH:

E is CH;

R⁰ is hydrogen;

R¹ is selected from methyl, ethyl, cyclopropyl, methoxyethyl and hydroxyethyl;

R¹⁶ is selected from methyl and ethyl;

R¹⁵ is selected from C(O)NH₂, C(O)NH(CH₂)₂OH and C(O)NH(CH₂)₂NH₂;

R⁴ is fluorine;

R⁵ is chlorine; and

R³ is selected from:
phenyl optionally substituted with one or two substituents selected from fluorine, chlorine, cyano, amino, mesylamino, acetylamino, methyl, hydroxymethyl, methoxy, cyanomethyl and oxazolyl;
pyridyl optionally substituted with amino or carbamoyl; and
dihydrobenzofuranyl.

1.97 A compound according to Embodiment 1.95 wherein:

A is CH:

E is CH;

R⁰ is hydrogen;

R¹ is selected from methyl, ethyl, cyclopropyl and methoxyethyl;

R¹⁶ is selected from methyl and ethyl;

R¹⁵ is C(O)NH₂;

R⁴ is fluorine;

R⁵ is chlorine; and

R³ is selected from:
phenyl optionally substituted with one or two substituents selected from fluorine, cyano, amino, acetylamino and methyl; and
pyridyl optionally substituted with amino or carbamoyl.

1.97A A compound according to Embodiment 1.95B wherein:

A is CH:

E is CH;

R⁰ is hydrogen;

R¹ is selected from ethyl and cyclopropyl;

R¹⁶ is methyl;

R¹⁵ is selected from C(O)NH₂ and C(O)NH(CH₂)₂NH₂;

R⁴ is fluorine;

R⁵ is chlorine; and

R³ is selected from:
unsubstituted phenyl or hydroxymethylphenyl; and
aminopyridyl.

1.98 A compound according to Embodiment 1.1 wherein:

A is CH;

E is CH;

R⁰ is hydrogen or C₁₋₂ alkyl;

R¹ is selected from:
C₁₋₅ alkyl unsubstituted or substituted with a substituent selected from:
   amino;
   hydroxy;
   methoxy;
   fluorine;
   isopropylamino;
   pyridylaminocarbonyl; and
   C(O)NH₂;
tetrahydropyridyl;
pyridyl;
piperidinyl;
piperidinylmethyl;
cyclohexenyl;
cyclopropyl;
tetrahydrofuranyl;
tetrahydropyranyl;
tetrahydropyranylmethyl; and
dihydroimidazolyl;

R² is selected from hydrogen and a group R^{2a};

R^{2a} is selected from:
C₁₋₃ alkyl optionally substituted with:
   a five membered monocyclic heteroaryl group containing one or two nitrogen ring members, wherein the heteroaryl group is optionally substituted with one or two methyl or ethyl groups;
   a four to six membered saturated monocyclic heterocyclic group containing a single nitrogen heteroatom ring member
   cyclopropyl;
   indolyl;
   pyridyl;
   hydroxy;
   SH;
   cyano; and
   methoxy;
allyl;
dihydroxypropyl;
C₃₋₆ cycloalkyl optionally substituted with amino;
piperidinyl;
aminomethylpyrimidinyl;
CH(R¹⁷)(CH₂)ₐC(O)NR^{18a}R^{18b} where a is 0 or 1; R¹⁷ is hydrogen, C₁₋₃ alkyl or cyclopropyl; R^{18a} is hydrogen or methyl and R^{18b} is selected from:
   hydrogen;
   methyl;
   cyclopropyl;
   cyanomethyl;
   hydroxy-C₂₋₄ alkyl;
   pyridyl;
   CH₂C(O)OCH₃;
   CH₂C(O)NH₂;
   amino;
   methoxy;
   a four to six membered saturated monocyclic heterocyclic ring containing a single heteroatom ring member selected from O and N;
   aminocyclobutyl;
   benzylaminoethyl;
or NR^{18a}R^{18b} forms a piperazine or diazepine ring;
pyridyl optionally substituted with amino;
tetrahydroisoquinolinyl;
dihydroisoindolyl; and
imidazolyl;

R³ is selected from:
unsubstituted phenyl;
phenyl substituted with one or two substituents selected from:
   —(CH₂)_{y}NHSO₂CH₃ where y is 0 or 1;
   C₁₋₂ alkyl optionally substituted with cyano, hydroxy or methoxyl or with one or more fluorine atoms;
   C₁₋₂ alkoxy
   pyrrolidinylcarbonyl;
   C(O)NHR¹⁹; where R¹⁹ is hydrogen or C₁₋₂ alkyl optionally substituted with cyano;
   C(O)NR²⁰R²¹ where R²⁰ is methyl and R²¹ is pyrazol-4-ylmethyl or 1-benzylpyrazol-4-ylmethyl;
   —CH(CH₃)OC(O)NHCH₂CH₃;
   CH₂OC(O)NHCH₂Cyp where Cyp is cyclopropyl;
   halogen;
   C(O)NH₂
   acetylamino;
   nitro;
   cyano;
   amino wherein the amino is optionally substituted with one or two C₁₋₂ alkyl groups;
   C₁₋₂ alkylsulphonyl;
   —NH(CO)NHCH₂CF₃;
   —CH₂NHC(O)CH₃;
   methyloxadiazolyl;
   oxazolyl;
   —SO₂NHCH₃;
   cyclopropyl optionally substituted with cyano or hydroxymethyl;
   CH═N—OH;
   ethynyl;
pyridine unsubstituted or substituted with a substituent selected from amino, acetylamino, chlorine, cyano, methyl, C(O)NH₂ and hydroxymethyl;
pyridazine substituted with chorine;
dihydrobenzofuran;
dihydroindole substituted with two methyl groups; and
pyridone;
R⁴ is selected from fluorine and chlorine; and
R⁵ is selected from fluorine; chlorine; methyl and ethyl.

1.98A A compound according to Embodiment 1.1 wherein:

A is CH;

E is CH;

R⁰ is hydrogen or C₁₋₂ alkyl;

R¹ is selected from:
C₁₋₅ alkyl unsubstituted or substituted with a substituent selected from:
   amino;
   hydroxy;
   methoxy;
   fluorine;
   isopropylamino;
   pyridylaminocarbonyl; and
   C(O)NH₂;
tetrahydropyridyl;
pyridyl;
piperidinyl;
piperidinylmethyl;
cyclohexenyl;
cyclopropyl;
tetrahydrofuranyl;
tetrahydropyranyl;
tetrahydropyranylmethyl; and
dihydroimidazolyl;

R² is selected from hydrogen and a group R^{2a};

R^{2a} is selected from:
C₁₋₃ alkyl optionally substituted with:
   a five membered monocyclic heteroaryl group containing one or two nitrogen ring members, wherein the heteroaryl group is optionally substituted with one or two methyl or ethyl groups;
   a four to six membered saturated monocyclic heterocyclic group containing a single nitrogen heteroatom ring member
   cyclopropyl;
   indolyl;
   pyridyl;
   hydroxy;
   SH;
   cyano; and
   methoxy;
allyl;
dihydroxypropyl;
C₃₋₆ cycloalkyl optionally substituted with amino;
piperidinyl;
aminomethylpyrimidinyl;
CH(R¹⁷)(CH₂)ₐC(O)NR^{18a}R^{18b} where a is 0 or 1; R¹⁷ is hydrogen, C₁₋₃ alkyl or cyclopropyl;
R^{18a} is hydrogen or methyl and R^{18b} is selected from:
   hydrogen;
   methyl;
   cyclopropyl;
   cyanomethyl;
   hydroxy-C₂₋₄ alkyl;
   pyridyl;
   CH₂C(O)OCH₃;
   CH₂C(O)NH₂;
   amino;
   methoxy;
   a four to six membered saturated monocyclic heterocyclic ring containing a single heteroatom ring member selected from O and N;
   aminocyclobutyl;
   benzylaminoethyl;
or NR^{18a}R^{18b} forms a piperazine or diazepine ring;
pyridyl optionally substituted with amino;
tetrahydroisoquinolinyl;
dihydroisoindolyl; and
imidazolyl;

R³ is selected from:
unsubstituted phenyl;
phenyl substituted with one or two substituents selected from:
   —(CH₂)_{y}NHSO₂CH₃ where y is 0 or 1;
   C₁₋₂ alkyl optionally substituted with cyano, hydroxy or methoxyl or with one or more fluorine atoms;
   C₁₋₂ alkoxy
   pyrrolidinylcarbonyl;
   C(O)NHR¹⁹; where R¹⁹ is hydrogen or C₁₋₂ alkyl optionally substituted with cyano;
   C(O)NR²⁰R²¹ where R²⁰ is methyl and R²¹ is pyrazol-4-ylmethyl or 1-benzylpyrazol-4-ylmethyl;
   —CH(CH₃)OC(O)NHCH₂CH₃;
   CH₂OC(O)NHCH₂Cyp where Cyp is cyclopropyl;
   halogen;
   C(O)NH₂
   acetylamino;
   nitro;
   cyano;
   amino wherein the amino is optionally substituted with one or two C₁₋₂ alkyl groups;
   acetylamino;
   dimethylureido;
   C₁₋₂ alkylsulphonyl;
   —NH(CO)NHCH₂CF₃;
   —CH₂NHC(O)CH₃;
   methyloxadiazolyl;
   oxazolyl;
   pyrazolyl;
   —SO₂NHCH₃;
   cyclopropyl optionally substituted with cyano or hydroxymethyl;
   CH═N—OH;
   ethynyl;
pyridine unsubstituted or substituted with a substituent selected from amino, acetylamino, chlorine, cyano, methyl, C(O)NH₂ and hydroxymethyl;
pyrimidine optionally substituted with amino;
pyridazine optionally substituted with chorine;
pyrazine optionally substituted with carboxy, C(O)NH₂ or amino;
oxadiazole substituted with methyl;
thiadiazole substituted with methyl;
dihydrobenzoxazine optionally substituted with oxo;
2,3-dihydro-benzo[1,4]dioxine;
benzothiazole optionally substituted with amino;
pyridothiazole
dihydrobenzofuran;
dihydroindole substituted with two methyl groups; and
pyridone;
R⁴ is selected from fluorine and chlorine; and
R⁵ is selected from fluorine; chlorine; methyl and ethyl.

1.99 A compound according to Embodiment 1.98 wherein:

A is CH;

E is CH;

R⁰ is hydrogen or ethyl;

R¹ is selected from:
C₁₋₅ alkyl unsubstituted or substituted with a substituent selected from:
   amino;
   hydroxy;
   methoxy;
   fluorine;
   isopropylamino;
   pyridylaminocarbonyl; and
   C(O)NH₂;
tetrahydropyridyl;
pyridyl;
piperidinyl;
piperidinylmethyl;
piperidinyl;
cyclohexenyl;
cyclopropyl;
tetrahydrofuranyl;
tetrahydropyranyl;
tetrahydropyranylmethyl; and
dihydroimidazolyl;

R² is selected from hydrogen and a group R^{2a};

R^{2a} is selected from:
C₁₋₃ alkyl optionally substituted with:
   pyrrolyl;
   pyrazolyl;
   imidazolyl wherein the imidazolyl is optionally substituted with one or two methyl or ethyl groups;
   cyclopropyl;
   azetidinyl;
   piperidinyl;
   indolyl;
   pyridyl;
   hydroxy;
   SH;
   cyano; and
   methoxy;
allyl;
dihydroxypropyl;
cyclobutyl;
cyclopentyl;
aminocyclohexyl;
aminocyclobutyl;
piperidinyl;
aminomethylpyrimidinyl;
CH(R¹⁷)(CH₂)ₐC(O)NR^{18a}R^{18b} where a is 0 or 1; R¹⁷ is hydrogen, C₁₋₃ alkyl or cyclopropyl;
R¹⁸ is hydrogen or methyl and R^{18b} is selected from:
   hydrogen;
   methyl;
   cyclopropyl;
   dimethylaminoethyl;
   ethylaminoethyl;
   cyanomethyl;
   hydroxy-C₂₋₄ alkyl;
   pyridyl;
   CH₂C(O)OCH₃;
   CH₂C(O)NH₂;
   amino;
   methoxy;
   oxetanyl;
   azetidinyl;
   aminocyclobutyl;
   pyrrolidinyl;
   piperidinyl;
   benzylaminoethyl;
or NR^{18a}R^{18b} forms a piperazine or diazepine ring;
pyridyl optionally substituted with amino;
tetrahydroisoquinolinyl;
dihydroisoindolyl; and
imidazolyl;

wherein at least one of R¹ and R² is other than hydrogen;

R³ is selected from:
unsubstituted phenyl;
phenyl substituted with one substituent selected from:
   —(CH₂)_{y}NHSO₂CH₃ where y is 0 or 1;
   ethyl;
   hydroxymethyl;
   hydroxyethyl;
   methoxyethyl;
   pyrrolidinylcarbonyl;
   C(O)NHR¹⁹; where R¹⁹ is hydrogen or cyanoethyl;
   C(O)NR²⁰R²¹ where R²⁰ is methyl and R²¹ is pyrazol-4-ylmethyl or 1-benzylpyrazol-4-ylmethyl;
   —CH(CH₃)OC(O)NHCH₂CH₃;
   CH₂OC(O)NHCH₂Cyp where Cyp is cyclopropyl;
   fluorine;
   chlorine;
   nitro;
   cyano;
   dimethylamino;
   cyanomethyl;
   trifluoromethyl;
   methylsulphonyl;
   —NH(CO)NHCH₂CF₃;
   —CH₂NHC(O)CH₃;
   methyloxadiazolyl;
   oxazolyl;
   —SO₂NHCH₃,
   cyanocyclopropyl;
   hydroxymethylcyclopropyl;
   CH═N—OH;
   ethynyl;
disubstituted phenyl wherein the two substituents are selected from cyano, fluorine, chlorine, methyl, methoxy, nitro, oxazolyl, C(O)NH₂, trifluoromethyl, acetylamino and amino;
pyridine unsubstituted or substituted with a substituent selected from amino, acetylamino, chlorine, cyano, methyl, C(O)NH₂ and hydroxymethyl;
pyridazine substituted with chorine;
dihydrobenzofuran;
dihydroindole substituted with two methyl groups; and
pyridone;
R⁴ is selected from fluorine and chlorine; and
R⁵ is selected from fluorine; chlorine; methyl and ethyl.

1.99A A compound according to Embodiment 1.98A wherein:

A is CH;

E is CH;

R⁰ is hydrogen or ethyl;

R¹ is selected from:
C₁₋₅ alkyl unsubstituted or substituted with a substituent selected from:
   amino;
   hydroxy;
   methoxy;
   fluorine;
   isopropylamino;
   pyridylaminocarbonyl; and
   C(O)NH₂;
tetrahydropyridyl;
pyridyl;
piperidinyl;
piperidinylmethyl;
piperidinyl;
cyclohexenyl;
cyclopropyl;
tetrahydrofuranyl;
tetrahydropyranyl;
tetrahydropyranylmethyl; and
dihydroimidazolyl;

R² is selected from hydrogen and a group R^{2a};

R^{2a} is selected from:
C₁₋₃ alkyl optionally substituted with:
   pyrrolyl;
   pyrazolyl;
   imidazolyl wherein the imidazolyl is optionally substituted with one or two methyl or ethyl groups;
   cyclopropyl;
   azetidinyl;
   piperidinyl;
   indolyl;
   pyridyl;
   hydroxy;
   SH;
   cyano; and
   methoxy;
allyl;
dihydroxypropyl;
cyclobutyl;
cyclopentyl;
aminocyclohexyl;
aminocyclobutyl;
piperidinyl;
aminomethylpyrimidinyl;
CH(R¹⁷)(CH₂)ₐC(O)NR^{18a}R^{18b} where a is 0 or 1; R¹⁷ is hydrogen, C₁₋₃ alkyl or cyclopropyl;
R¹⁸ is hydrogen or methyl and R^{18b} is selected from:
   hydrogen;
   methyl;
   cyclopropyl;
   dimethylaminoethyl;
   ethylaminoethyl;
   cyanomethyl;
   hydroxy-C₂₋₄ alkyl;
   pyridyl;
   CH₂C(O)OCH₃;
   CH₂C(O)NH₂;
   amino;
   methoxy;
   oxetanyl;
   azetidinyl;
   aminocyclobutyl;
   pyrrolidinyl;
   piperidinyl;
   benzylaminoethyl;
or NR^{18a}R^{18b} forms a piperazine or diazepine ring;
pyridyl optionally substituted with amino;
tetrahydroisoquinolinyl;
dihydroisoindolyl; and
imidazolyl;

wherein at least one of R¹ and R² is other than hydrogen;

R³ is selected from:
unsubstituted phenyl;
phenyl substituted with one substituent selected from:
   —(CH₂)_{y}NHSO₂CH₃ where y is 0 or 1;
   ethyl;
   hydroxymethyl;
   hydroxyethyl;
   methoxyethyl;
   pyrrolidinylcarbonyl;
   C(O)NHR¹⁹; where R¹⁹ is hydrogen or cyanoethyl;
   C(O)NR²⁰R²¹ where R²⁰ is methyl and R²¹ is pyrazol-4-ylmethyl or 1-benzylpyrazol-4-ylmethyl;
   —CH(CH₃)OC(O)NHCH₂CH₃;
   CH₂OC(O)NHCH₂Cyp where Cyp is cyclopropyl;
   fluorine;
   chlorine;
   nitro;
   cyano;
   amino
   dimethylamino;
   acetylamino;
   dimethylureido;
   cyanomethyl;
   trifluoromethyl;
   methylsulphonyl;
   —NH(CO)NHCH₂CF₃;
   —CH₂NHC(O)CH₃;
   methyloxadiazolyl;
   oxazolyl;
   pyrazolyl;
   —SO₂NHCH₃;
   cyanocyclopropyl;
   hydroxymethylcyclopropyl;
   CH═N—OH;
   ethynyl;
disubstituted phenyl wherein the two substituents are selected from cyano, fluorine, chlorine, methyl, methoxy, nitro, oxazolyl, C(O)NH₂, methylcarbamoyl, dimethylcarbamoyl, morpholinylcarbonyl, trifluoromethyl, acetylamino and amino;
pyridine unsubstituted or substituted with a substituent selected from amino, dimethylamino, acetylamino, chlorine, cyano, methyl, C(O)NH₂ and hydroxymethyl;
pyrimidine optionally substituted with amino;
pyridazine optionally substituted with chorine;
pyrazine optionally substituted with carboxy, C(O)NH₂ or amino;
oxadiazole substituted with methyl;
thiadiazole substituted with methyl;
dihydrobenzofuran;
dihydroindole substituted with two methyl groups;
dihydrobenzoxazine optionally substituted with oxo;
2,3-dihydro-benzo[1,4]dioxine;
benzothiazole optionally substituted with amino;
pyridothiazole; and
pyridone;
R⁴ is selected from fluorine and chlorine; and
R⁵ is selected from fluorine; chlorine; methyl and ethyl.

1.100 A compound according to Embodiment 1.1 having the formula (6):

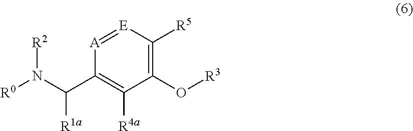

or a salt, N-oxide or tautomer thereof, wherein A, E, R⁰, R^{1a}, R², R³, R^{4a} and R⁵ are as defined in any one of Embodiments 1.1 to 1.56G and 1.57 to 1.99.

1.101 A compound according to Embodiment 1.100 having the stereochemical form (6a):

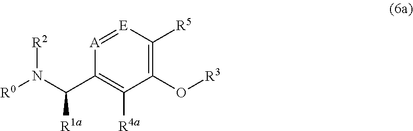

1.102 A compound according to Embodiment 1.100 having the stereochemical form (6b):

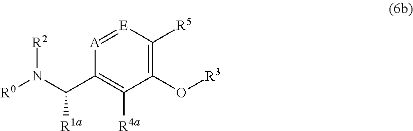

1.102A A compound according to Embodiment 1.100 having the formula (7):

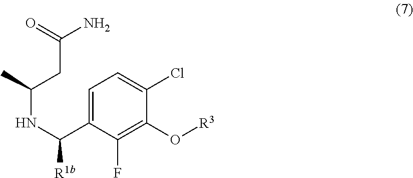

or a salt, N-oxide, tautomer or stereoisomer thereof,

wherein R^{1b} is selected from ethyl and cyclopropyl and R³ is as defined in any one of Embodiments 1.1 to 1.102.

1.103 A compound according to any one of Embodiments 1.1 to 1.102 which is other than (S)-3-[(R)-1-(4-chloro-2-fluoro-3-phenoxy-phenyl)-propylamino]-N-isopropyl-butyramide.

1.103A A compound according to any one of Embodiments 1.1 to 1.102 which is other than (S)-3-[(R)-1-(4-chloro-2-fluoro-3-phenoxy-phenyl)propylamino]-2-hydroxy-1,1,dimethylethyl-butyramide.

1.104 A compound according to any one of Embodiments 1.1 to 1.103A having a molecular weight of up to 1000.

1.104A A compound according to Embodiment 1.104 having a molecular weight of less than 750.

1.105 A compound according to Embodiment 1.104A having a molecular weight of less than 700.

1.106 A compound according to Embodiment 1.105 having a molecular weight of less than 650.

1.107 A compound according to Embodiment 1.106 having a molecular weight of less than 600 or less than 550.

1.108 A compound according to Embodiment 1.107 having a molecular weight of less than 525, for example, 500 or less.

1.109 A compound selected from the title compounds of any of Examples 1 to 518.

### DEFINITIONS

In this application, the following definitions apply, unless indicated otherwise.

References herein to formula (1) include formula (0) unless the context indicates otherwise.

The term “treatment” as used herein in relation to hepatitis C virus infections is used in a general sense to describe any form of intervention where a compound is administered to a subject suffering from, or at risk of suffering from, or potentially at risk of suffering from infection with HCV. Thus the term treatment covers both preventative (prophylactic) treatment (e.g. where there may be a risk of infection but no actual infection has been detected) and treatment where a subject has become infected with HCV. When a subject (e.g. a human subject) has become infected, the treatment may comprise management of the infection or elimination of the infection.

The term “subject” as used herein may refer to a human subject or a non-human subject. In a preferred embodiment, the subject is a human subject. Where the subject is a non-human subject, it may be for example another mammalian species or an avian species. The mammalian species may be, for example, a domestic animal such as a dog or cat, or farmed animals such as cattle, pigs, sheep, horses and goats. Thus, the compounds of the invention may be used in human or veterinary medicine.

As used herein, the term “combination”, as applied to two or more compounds andor agents (also referred to herein as the components), is intended to define material in which the two or more compoundsagents are associated. The terms “combined” and “combining” in this context are to be interpreted accordingly.

The association of the two or more compoundsagents in a combination may be physical or non-physical. Examples of physically associated combined compoundsagents include:
compositions (e.g. unitary formulations) comprising the two or more compoundsagents in admixture (for example within the same unit dose);
compositions comprising material in which the two or more compoundsagents are chemicallyphysicochemically linked (for example by crosslinking, molecular agglomeration or binding to a common vehicle moiety);
compositions comprising material in which the two or more compoundsagents are chemicallyphysicochemically co-packaged (for example, disposed on or within lipid vesicles, particles (e.g. micro- or nanoparticles) or emulsion droplets);
pharmaceutical kits, pharmaceutical packs or patient packs in which the two or more compoundsagents are co-packaged or co-presented (e.g. as part of an array of unit doses);

Examples of non-physically associated combined compoundsagents include:
material (e.g. a non-unitary formulation) comprising at least one of the two or more compoundsagents together with instructions for the extemporaneous association of the at least one compound to form a physical association of the two or more compoundsagents;
material (e.g. a non-unitary formulation) comprising at least one of the two or more compoundsagents together with instructions for combination therapy with the two or more compoundsagents;
material comprising at least one of the two or more compoundsagents together with instructions for administration to a patient population in which the other(s) of the two or more compoundsagents have been (or are being) administered;
material comprising at least one of the two or more compoundsagents in an amount or in a form which is specifically adapted for use in combination with the other(s) of the two or more compoundsagents.

As used herein, the term “combination therapy” is intended to define therapies which comprise the use of a combination of two or more compoundsagents (as defined above). Thus, references to “combination therapy”, “combinations” and the use of compoundsagents “in combination” in this application may refer to compoundsagents that are administered as part of the same overall treatment regimen. As such, the posology of each of the two or more compoundsagents may differ: each may be administered at the same time or at different times. It will therefore be appreciated that the compoundsagents of the combination may be administered sequentially (e.g. before or after) or simultaneously, either in the same pharmaceutical formulation (i.e. together), or in different pharmaceutical formulations (i.e. separately). Administration simultaneously in the same formulation would involve administration of a unitary formulation whereas administration simultaneously in different pharmaceutical formulations would involve non-unitary formulations. The posologies of each of the two or more compoundsagents in a combination therapy may also differ with respect to the route of administration.

As used herein, the term “pharmaceutical kit” defines an array of one or more unit doses of a pharmaceutical composition together with dosing means (e.g. measuring device) andor delivery means (e.g. inhaler or syringe), optionally all contained within common outer packaging. In pharmaceutical kits comprising a combination of two or more compoundsagents, the individual compoundsagents may unitary or non-unitary formulations. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. In pharmaceutical packs comprising a combination of two or more compoundsagents, the individual compoundsagents may unitary or non-unitary formulations. The unit dose(s) may be contained within a blister pack. The pharmaceutical pack may optionally further comprise instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions

The term “acyclic hydrocarbon group” (as in “acyclic C₁₋₈ hydrocarbon group” or “acyclic C₁₋₆ hydrocarbon group” or “acyclic C₁₋₅ hydrocarbon group”) refers to a non-cyclic group consisting of carbon and hydrogen atoms. The hydrocarbon group may be fully saturated or may contain one or more carbon-carbon double bonds or carbon-carbon triple bonds, or mixtures of double and triple bonds. The hydrocarbon group may be a straight chain or branched chain group.

Examples of acyclic C₁₋₈ hydrocarbon groups are alkyl, alkenyl and alkynyl groups.

In each instance where the term “acyclic C₁₋₈ hydrocarbon group” appears in any of Embodiments 1.1 to 1.109, a subset of acyclic C₁₋₈ hydrocarbon groups consists of C₁₋₈ alkyl, C₂₋₈ alkenyl and C₂₋₈ alkynyl groups. A particular subset of acyclic C₁₋₈ hydrocarbon groups consists of C₁₋₈ alkyl groups.

In each instance where the term “acyclic C₁₋₆ hydrocarbon group” appears in any of Embodiments 1.1 to 1.109, a subset of acyclic C₁₋₆ hydrocarbon groups consists of C₁₋₆ alkyl, C₂₋₆ alkenyl and C₂₋₆ alkynyl groups. A particular subset of acyclic C₁₋₆ hydrocarbon groups consists of C₁₋₆ alkyl groups.

In each instance where the term “acyclic C₁₋₅ hydrocarbon group” appears in any of Embodiments 1.1 to 1.109, a subset of acyclic C₁₋₅ hydrocarbon groups consists of C₁₋₅ alkyl, C₂₋₅ alkenyl and C₂₋₅ alkynyl groups. A particular subset of acyclic C₁₋₅ hydrocarbon groups consists of C₁₋₅ alkyl groups.

A further subset of acyclic C₁₋₈ hydrocarbon groups or acyclic C₁₋₆ hydrocarbon groups or acyclic C₁₋₅ hydrocarbon groups consists of C₁₋₄ alkyl, C₂₋₄ alkenyl and C₂₋₄ alkynyl groups. A particular subset consists of C₁₋₄ alkyl groups.

Within each of Embodiments 1.1 to 1.109, preferred subsets of acyclic C₁₋₈ hydrocarbon groups or acyclic C₁₋₆ hydrocarbon groups or acyclic C₁₋₅ hydrocarbon groups are C₁₋₈ alkyl groups, or C₁₋₆ alkyl groups, or C₁₋₅ alkyl groups or C₁₋₄ alkyl groups. One particular sub-set of alkyl groups consists of methyl, ethyl, n-propyl, isopropyl, n-butyl, isobutyl and tert-butyl. Another particular subset of alkyl groups consists of methyl, ethyl and isopropyl groups.

The term “unbranched (straight chain) alkyl group” refers to an alkyl group which is of the formula —(CH₂)ₙ—H where n is an integer. In the case of a C₁₋₆ alkyl group, n is an integer from 1 to 6. Where stated, the alkyl group may be optionally substituted with one or more defined substituents. In a substituted alkyl group, one or more of the hydrogen atoms may be replaced with a defined substituent.

References to a “monocyclic carbocyclic or heterocyclic group of 3 to 7 ring members cover non-aromatic and aromatic rings, unless the context indicates otherwise. Non-aromatic rings can be fully saturated (i.e. they contain no carbon-carbon or carbon-nitrogen multiple bonds) or partially unsaturated (i.e. they may contain one or in some cases two carbon-carbon or carbon-nitrogen double bonds). Unless indicated otherwise, the monocyclic or heterocyclic group of 3 to 7 ring members has 0, 1 or 2 heteroatom ring members selected from O, N and S.

An example of an aromatic ring is phenyl.

When the monocyclic or heterocyclic group is aromatic, typically it is a five or six membered ring.

Examples of five membered aromatic heterocyclic (heteroaryl) groups include but are not limited to pyrrole, furan, thiophene, imidazole, oxazole, isoxazole, thiazole, isothiazole and pyrazole.

Examples of six membered aromatic heterocyclic (heteroaryl) groups include but are not limited to pyridine, pyridone, pyrazine, pyridazine, pyrimidine and pyrimidone groups.

Examples of non-aromatic monocyclic carbocyclic groups of 3 to 7 ring members are C₃₋₇ cycloalkyl and C₃₋₇ cycloalkenyl groups such as cyclopropyl, cyclobutyl, cyclopentyl, cyclohexyl, cycloheptyl and cyclohexenyl.

Examples of non-aromatic monocyclic heterocyclic groups of 3 to 7 ring members are aziridine, azetidine, pyrrolidine, piperidine, azepine, piperazine, morpholine, thiomorpholine, tetrahydrofuran, tetrahydropyran, dihydropyran, dihydrofuran, dihydrothiazole, tetrahydrothiophene, dioxane, imidazoline, oxazoline, thiazoline, pyrazoline and pyrazolidine.

In formula (1), R² can be a bicyclic heterocyclic group of 9 or 10 ring members, of which 1 or 2 ring members are nitrogen atoms, one of the rings of the bicyclic heterocyclic group being a non-aromatic nitrogen-containing ring. Typically, one ring of the bicyclic heterocyclic group is aromatic. The aromatic ring may be a five membered or six membered ring. Thus, the bicyclic heterocyclic group can consist of (a) a six-membered aromatic ring fused to a six membered non-aromatic ring; or (b) a six-membered aromatic ring fused to a six membered non-aromatic ring; or (c) a five membered aromatic ring fused to a six membered non-aromatic ring. The six membered aromatic ring in (a) or (b) may be, for example, a benzene or pyridine ring. The five membered aromatic ring in (c) may be, for example, a pyrrole, thiophene or furan ring.

Examples of the bicyclic heterocyclic groups are tetrahydroquinoline, tetrahydroisoquinoline, dihydroindole, dihydroisoindole, dihydrobenzofuran, dihydrobenzopyran, dihydrobenzothiophene and aza-analogues thereof in which the benzene ring is replaced by a pyridine ring.

The term “bicyclic heteroaryl” as used herein refers to bicyclic ring systems in which both rings are aromatic.

The term “N-linked substituent” as used herein refers to a nitrogen atom-containing substituent such as an amino, methylamino, methylamino, pyrrolidinyl or morpholinyl group which is attached through the nitrogen atom.

The term “alkanoyl” as used herein refers to the acyl residue of an alkanoic acid. Examples of C₁₋₄ alkanoyl groups are formyl, acetyl, propanoyl and butanoyl.

The term “non-aromatic heterocyclic group having a total of 4 to 7 ring members of which 1 or 2 are nitrogen atoms and the others are carbon atoms” (e.g. as used in the definition of NR¹⁰R¹¹ above) refers to both fully saturated and partially unsaturated groups, but typically the groups are fully saturated; i.e. they contain no carbon-carbon or carbon-nitrogen multiple bonds. Examples of the non-aromatic heterocyclic groups are azetidine, pyrrolidine, piperidine, azepine, piperazine, imidazoline, pyrazoline and pyrazolidine groups.

### Salts and Free Bases

Many compounds of the formula (1) can exist in the form of salts, for example acid addition salts or, in certain cases salts of organic and inorganic bases such as carboxylate, sulfonate and phosphate salts. All such salts are within the scope of this invention, and references to compounds of the formula (1) include the salt forms of the compounds.

The salts are typically acid addition salts.

Alternatively, the compounds can exist in the free base form.

Accordingly, the invention also provides the following Embodiments 1.200 to 1.202:

1.200 A compound according to any one of Embodiments 1.1 to 1.109 which is in the form of a salt.

1.200A A compound according to any one of Embodiments 1.1 to 1.109 which is in the form of a free base.

1.201 A compound according to Embodiment 1.200 wherein the salt is an acid addition salt.

1.202 A compound according to Embodiment 1.200 or Embodiment 1.201 wherein the salt is a pharmaceutically acceptable salt.

The salts of the present invention can be synthesized from the parent compound that contains a basic or acidic moiety by conventional chemical methods such as methods described in Pharmaceutical Salts Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used.

Acid addition salts (as defined in Embodiment 1.201) may be formed with a wide variety of acids, both inorganic and organic. Examples of acid addition salts falling within Embodiment 1.201 include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, aspartic (e.g. L-aspartic), hydrochloric, hydriodic, phosphoric, nitric, sulfuric, citric, lactic, succinic, maleic, malic, isethionic, fumaric, benzenesulfonic, toluenesulfonic, methanesulfonic (mesylate), ethanesulfonic, naphthalenesulfonic, valeric, acetic, propanoic, butanoic, malonic, glucuronic and lactobionic acids. One particular salt is the hydrochloride salt.

If the compound is anionic, or has a functional group which may be anionic (e.g., —COOH may be —COO⁻), then a salt may be formed with an organic or inorganic bases, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (1) contain an amine function, these may form quaternary ammonium salts, for example by reaction with an alkylating agent according to methods well known to the skilled person. Such quaternary ammonium compounds are within the scope of formula (1).

The compounds of the invention may exist as mono- or di-salts depending upon the pKa of the acid from which the salt is formed.

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

In one embodiment of the invention, there is provided a pharmaceutical composition comprising a solution (e.g. an aqueous solution) containing a compound of the formula (1) and sub-groups and examples thereof as described herein in the form of a salt in a concentration of greater than 10 mgml, typically greater than 15 mgml and preferably greater than 20 mgml.

### N-Oxides

N-Oxides can be formed by treatment of the corresponding amine with an oxidizing agent such as hydrogen peroxide or a per-acid (e.g. a peroxycarboxylic acid), see for example Albini, A.; Pietra, S. Heterocyclic N-Oxides; CRC Press:Boca Raton, Fla., 1991, pp 31 More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Accordingly, the invention also provides:

1.203 A compound according to any one of Embodiments 1.1 to 1.109 which is in the form of an N-oxide.

### Tautomers

The compounds of the invention may exist in a number of different tautomeric forms and references to the compounds of formula (1) and their salts and N-oxides as defined in Embodiments 1.1 to 1.203 include all such forms.

For example, when R³ is a pyridine group substituted with hydroxy as shown below, the ring system may exhibit tautomerism between tautomers A and B.

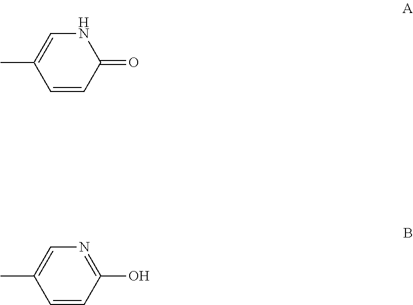

For the avoidance of doubt, where a compound can exist in one of several tautomeric forms and only one is specifically described or shown, all others are nevertheless embraced by Embodiments 1.1 to 1.203.

Accordingly, in another embodiment (Embodiment 1.204), the invention provides a tautomer of a compound according to any one of Embodiments 1.1 to 1.203.

### Stereoisomers

Stereoisomers are isomeric molecules that have the same molecular formula and sequence of bonded atoms but which differ only in the three-dimensional orientations of their atoms in space.

The stereoisomers can be, for example, geometric isomers or optical isomers.

### Geometric Isomers

With geometric isomers, the isomerism is due to the different orientations of an atom or group about a double bond, as in cis and trans (Z and E) isomerism about a carbon-carbon double bond, or cis and trans isomers about an amide bond, or syn and anti isomerism about a carbon nitrogen double bond (e.g. in an oxime), or rotational isomerism about a bond where there is restricted rotation, or cis and trans isomerism about a ring such as a cycloalkane ring.

Accordingly, in another embodiment (Embodiment 1.205), the invention provides a geometric isomer of a compound according to any one of Embodiments 1.1 to 1.204.

### Optical Isomers

Where compounds of the formula contain one or more chiral centres, and can exist in the form of two or more optical isomers, references to the compounds include all optical isomeric forms thereof (e.g. enantiomers, epimers and diastereoisomers), either as individual optical isomers, or mixtures (e.g. racemic mixtures) or two or more optical isomers, unless the context requires otherwise.

Accordingly, in another embodiment (Embodiment 1.206) the invention provides an optical isomeric form of a compound according to any one of Embodiments 1.1 to 1.205.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluoyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the invention exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers.

Accordingly, in another embodiment (Embodiment 1.207), the invention provides compositions containing a compound according to any one Embodiments 1.1 to 1.206 having one or more chiral centres, wherein at least 55% (e.g. at least 60%, 65%, 70%, 75%, 80%, 85%, 90% or 95%) of the compound of any one of Embodiments 1.1 to 1.206 is present as a single optical isomer (e.g. enantiomer or diastereoisomer).

In one general embodiment (Embodiment 1.208), 99% or more (e.g. substantially all) of the total amount of the compound (or compound for use) of any one of Embodiments 1.1 to 1.206 is present as a single optical isomer.

For example, in one embodiment (Embodiment 1.209) the compound is present as a single enantiomer.

In another embodiment (Embodiment 1.210), the compound is present as a single diastereoisomer.

The invention also provides mixtures of optical isomers, which may be racemic or non-racemic. Thus, the invention provides:

Embodiment 1.211 A compound according to any one of Embodiments 1.1 to 1.204 which is in the form of a racemic mixture of optical isomers.

Embodiment 1.212: A compound according to any one of Embodiments 1.1 to 1.204 which is in the form of a non-racemic mixture of optical isomers.

### Isotopes

The compounds of the invention as defined in any one of Embodiments 1.1 to 1.212 may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

In an analogous manner, a reference to a particular functional group also includes within its scope isotopic variations, unless the context indicates otherwise.

For example, a reference to an alkyl group such as an ethyl group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group).

The isotopes may be radioactive or non-radioactive. In one embodiment of the invention (Embodiment 1.213), the compound of any one of Embodiments 1.1 to 1.212 contains no radioactive isotopes. Such compounds are preferred for therapeutic use. In another embodiment (Embodiment 1.214), however, the compound of any one of Embodiments 1.1 to 1.212 may contain one or more radioisotopes. Compounds containing such radioisotopes may be useful in a diagnostic context.

### Solvates

Compounds of the formula (1) as defined in any one of Embodiments 1.1 to 1.214 may form solvates.

Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates.

Particularly preferred solvates are hydrates, and examples of hydrates include hemihydrates, monohydrates and dihydrates.

Accordingly, in further embodiments 1.215 and 1.216, the invention provides:

1.215 A compound according to any one of Embodiments 1.1 to 1.214 in the form of a solvate.

1.216 A compound according to Embodiment 1.215 wherein the solvate is a hydrate.

For a more detailed discussion of solvates and the methods used to make and characterise them, see Bryn et al., Solid-State Chemistry of Drugs, Second Edition, published by SSCI, Inc of West Lafayette, Ind., USA, 1999, ISBN 0-967-06710-3.

Alternatively, rather than existing as a hydrate, the compound of the invention may be anhydrous. Therefore, in another embodiment (Embodiment 1.217), the invention provides a compound as defined in any one of Embodiments 1.1 to 1.214 in an anhydrous form (e.g. anhydrous crystalline form).

### Crystalline and Amorphous Forms

The compounds of any one of Embodiments 1.1 to 1.217 may exist in a crystalline or non-crystalline (e.g. amorphous) state.

Whether or not a compound exists in a crystalline state can readily be determined by standard techniques such as X-ray powder diffraction (XRPD).

Crystals and their crystal structures can be characterised using a number of techniques including single crystal X-ray crystallography, X-ray powder diffraction (XRPD), differential scanning calorimetry (DSC) and infra red spectroscopy, e.g. Fourier Transform infra-red spectroscopy (FTIR). The behaviour of the crystals under conditions of varying humidity can be analysed by gravimetric vapour sorption studies and also by XRPD.

Determination of the crystal structure of a compound can be performed by X-ray crystallography which can be carried out according to conventional methods such as those described herein and as described in Fundamentals of Crystallography, C. Giacovazzo, H. L. Monaco, D. Viterbo, F. Scordari, G. Gilli, G. Zanotti and M. Catti, (International Union of CrystallographyOxford University Press, 1992 ISBN 0-19-855578-4 (pb), 0-19-85579-2 (hb)). This technique involves the analysis and interpretation of the X-ray diffraction of single crystal.

In an amorphous solid, the three dimensional structure that normally exists in a crystalline form does not exist and the positions of the molecules relative to one another in the amorphous form are essentially random, see for example Hancock et al. J. Pharm. Sci. (1997), 86, 1).

Accordingly, in further embodiments, the invention provides:

1.218 A compound according to any one of Embodiments 1.1 to 1.217 in a crystalline form.

1.219 A compound according to any one of Embodiments 1.1 to 1.217 which is:

(a) from 50% to 100% crystalline, and more particularly is at least 50% crystalline, or at least 60% crystalline, or at least 70% crystalline, or at least 80% crystalline, or at least 90% crystalline, or at least 95% crystalline, or at least 98% crystalline, or at least 99% crystalline, or at least 99.5% crystalline, or at least 99.9% crystalline, for example 100% crystalline.

1.220 A compound according to any one of Embodiments 1.1 to 1.217 which is in an amorphous form.

### Prodrugs

The compounds of the formula (1) as defined in any one of Embodiments 1.1 to 1.220 may be presented in the form of a pro-drug. By “prodrugs” is meant for example any compound that is converted in vivo into a biologically active compound of the formula (1), as defined in any one of Embodiments 1.1 to 1.220.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any hydroxyl groups present in the parent compound with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Accordingly, in another embodiment (Embodiment 1.221), the invention provides a pro-drug of a compound as defined in any one of Embodiments 1.1 to 1.219 wherein the compound contains a functional group which is convertable under physiological conditions to form a hydroxyl group or amino group.

### Complexes and Clathrates

Also encompassed by formula (1) in Embodiments 1.1 to 1.221 are complexes (e.g. inclusion complexes or clathrates with compounds such as cyclodextrins, or complexes with metals) of the compounds of Embodiments 1.1 to 1.221.

Accordingly, in another embodiment (Embodiment 1.222), the invention provides a compound according to any one of Embodiments 1.1 to 1.221 in the form of a complex or clathrate.

### Methods for the Preparation of Compounds of the Formula (1)

Compounds of the formula (1), as defined in Embodiments 1.0, 1.00 and 1.1 to 1.222, can be prepared in accordance with synthetic methods well known to the skilled person and as described herein. Reaction Schemes 1 to 10 below illustrate general methods for making the compounds of formula (1).

For example, they can be constructed through formation of the biaryl ether and benzylamine, by substitution at the benzylamine moiety and through additional modifications of intermediate molecules. The order of these steps can be varied providing that tolerant functional groups are present andor with relevant protecting groups (see Protective Groups in Organic Synthesis, Greene and Wuts, Wiley Interscience). The stereochemistry depicted in the reaction schemes set out below is by way of example only; each of the relevant stereoisomers can be synthesised using suitable reactantsreagents.

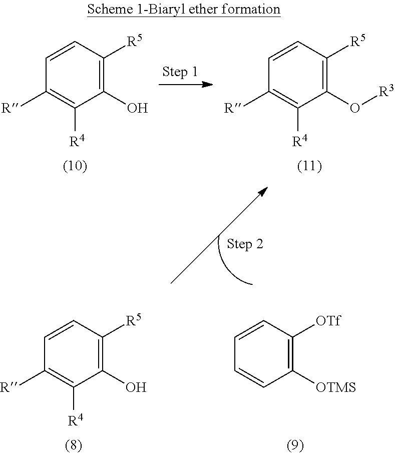

The introduction of the R³ group can take place either as the end step in the synthetic route to compounds of the formula (1) or, more usually, during one of the intermediate steps.

Scheme 1 illustrates two methods of forming an aryloxyheteroaryloxy ether bond. In Scheme 1, the moiety R″ can be a group:

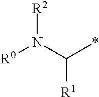

or a protected version thereof, where the asterisk indicates the point of attachment to the phenyl ring, or the moiety R″ can be a precursor group such as methyl which then undergoes further transformations to give the group R⁰R²NCH(R¹)—.

Step 1 in Scheme 1 makes use of a Chan-Lam coupling reaction in which an appropriately substituted phenol (8) is reacted with an aryl or heteroaryl boronic acid R³—B(OH)₂ using a suitable catalyst such as copper (II) actetate under basic conditions to give the biaryl compound (11).

In one set of particular reaction conditions, as used to prepare key intermediates in the synthesis of the exemplified compounds described in the experimental section below, the compound of formula (10) is reacted with the boronic acid R³—B(OH)₂ in dichloromethane in the presence of copper (II) acetate, pyridine, pyridine N-oxide and powdered 4 Å molecular sieves at room temperature. Particular examples of compounds of the formula (11) prepared by this route are those in which R″ is methyl.

In an alternative approach, as illustrated in Step 2 of Scheme 1, in situ trapping by a phenol of a reactive benzyne species generated from, for example, 2-(trimethylsilyl)phenyl trifluoromethane sulfonate will generate the compounds of interest. This reaction can be carried out by reacting a solution of the 2-trimethylsilyloxy triflate compound (9) in acetonitrile with the phenol (8) in the presence of caesium fluoride at room temperature, followed by quenching with potassium hydroxide.

As an alternative to Steps 1 and 2 in Scheme 1, the formation of aryloxy- and heteroaryloxy ethers can be achieved using an Ullman-type coupling of phenols with aryls or heteroaryls bearing a leaving group such as a halide or triflate using copper (I) salts under basic conditions. If the aryl or heteroaryl group is sufficiently electrophilic, SNAr chemistry can be used to produce the intermediates under basic conditions in a suitable solvent such as acetonitrile, dimethylsulfoxide or dimethylformamide typically at raised temperatures.

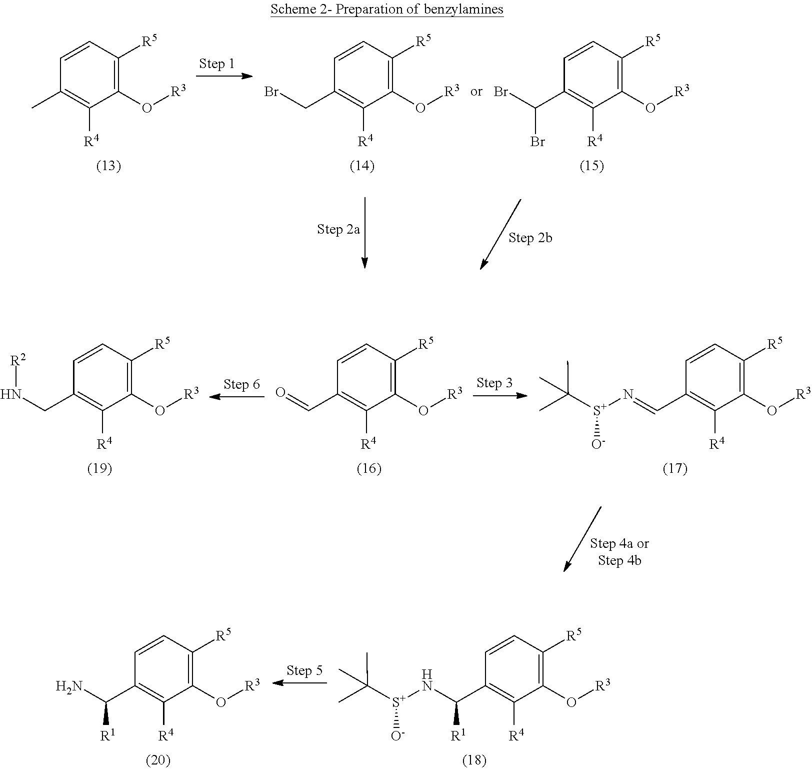

Compounds of the formula (11) in Scheme 1, wherein R″ is a methyl group, can be converted into optionally substituted benzylamine compounds of the formula (1) in a number of ways, examples of which are shown in Scheme 2.

In Scheme 2, a substituted toluene compound of formula (13) (which corresponds to a compound of formula (11) wherein R″ is methyl) can be converted in a series of steps via a benzaldehyde intermediate to give a substituted benzylamine.

In a first step (Step 1), the substituted toluene compound (13) is subjected to free radical bromination using an electrophilic bromine source (typically N-bromosuccinimide) and a free radical initiator (e.g. azobisisobutyronitrile (AlBN) or benzoyl peroxide). The bromination reaction is typically performed in a chlorinated solvent (e.g. carbon tetrachloride or dichloromethane) with heating (e.g. to a temperature of about 80° C.) under an inert atmosphere. Either the monobrominated product, compound (14), or the dibrominated product, compound (15), can be obtained from the bromination reaction depending on the number of equivalents of brominating agent used.

The bromo-compounds (14) and (15) can each be transformed into an aldehyde (16). In Step 2a, the monobromo-compound (14) can be treated with sodium bicarbonate in dimethylsulphoxide, preferably with heating to about 80 C, in order to oxidise the monobromide (14) to give the aldehyde (16).

In Step 2b, the dibromide (15) can be hydrolysed using silver nitrate in isopropyl alcohol, typically at room temperature, to give the aldehyde (16). The aldehyde (16) can then be used in a number of different synthetic conversions to give compounds of the formula (1).

In Scheme 2, Step 3, the aldehyde (16) is converted to the chiral sulphinylimine (17) by reaction with a chiral form of tert-butyl sulfinimide in the presence of a Lewis acid promoter such as titanium (IV) ethoxide. In Step 4a, the sulphinylimine intermediate (17) is then reacted with a nucleophilic reagent suitable for introducing the group R¹ or a precursor to the group R¹. For example, the intermediate (17) can be reacted at low temperature with a nucleophilic reactant such as a Grignard reagent (e.g. ethyl magnesium bromide), an alkyl, aryl or heteroaryl anion (such as isopropyl lithium, pyridin-3-yl lithium), or nitromethane (with tetra-n-butylammonium fluoride) to give the chiral sulphinamide (18), often as a mixture of diastereoisomers which can typically be readily separated by flash column chromatography.

In Step 5, the tert-butyl sulfinyl group is removed under acidic conditions (for example by treatment with a hydrohalic acid such as hydrochloric acid in a suitable solvent such as tetrahydrofuran, dioxane, ethyl acetate or methanol to give the α-substituted N-unsubstituted benzylamine (20).

Alternatively, in Step 4b, the sulfinimide (17) can be subjected to a transition metal catalysed coupling with a boronic acidester or a trifluoroborate salt. In one particular example of example of Step 4b, (N-Boc)-1,2,3,6-tetrahydropyridine-4-boronic acid pinacol ester can be coupled with the sulfinimide (17) using bis(acetonitrile)(1,5-cyclooctadiene)rhodium (I) tetrafluoroborate as a catalyst to give firstly an intermediate compound (18) and then, after removal of the tert-butyl sulfinyl group using HCl in dioxanemethanol, a compound of formula (20) wherein R¹ is a 1,2,3,6-tetrahydro-pyridin-4-yl group.

Additional functional group interconversions may be carried out on compounds of type (20). For example, when the group R¹ contains a high oxidation state group such as an alkene or nitro group, these can be reduced using catalytic hydrogenation or other metal mediated reducing conditions (such as tin in HCl or ironiron sulphate) to give the corresponding alkyl or amino group. Where the group R¹ contains an ester group, the ester group can be hydrolysed (e.g. with lithium hydroxide) and the resulting carboxylic acid converted to an amide by reaction with an amine and an amide coupling reagent (such as a combination of hydroxybenzotriazole and 1-ethyl-3-(3-dimethylaminopropyl)carbodiimide hydrochloride). Where R¹ contains an amine group, this can be reductively alkylated (e.g. with isopropyl ketone in the presence of sodium triacetoxyborohydride and acetic acid).

In Step 6 in Scheme 2, the benzaldehyde intermediate (16) is converted by a reductive amination step to the amine (19) by reacting the aldehyde (16) with an amine R²—NH₂ and a suitable reducing agent such as sodium triacetoxyborohydride, typically in tetrahydrofuran or a chlorinated solvent.

The reductive amination procedure can also be achieved in two steps by imine formation under dehydrating conditions where the aldehyde (16) and amine R²—NH₂ are refluxed (e.g. under Dean-Stark conditions) in the presence of catalytic acid (e.g. para-toluenesulfonic acid) or mixed with a Lewis acid in a non-protic solvent (e.g. titanium IV chloride in dichloromethane) followed by reduction with a suitable reducing agent such as sodium borohydride.

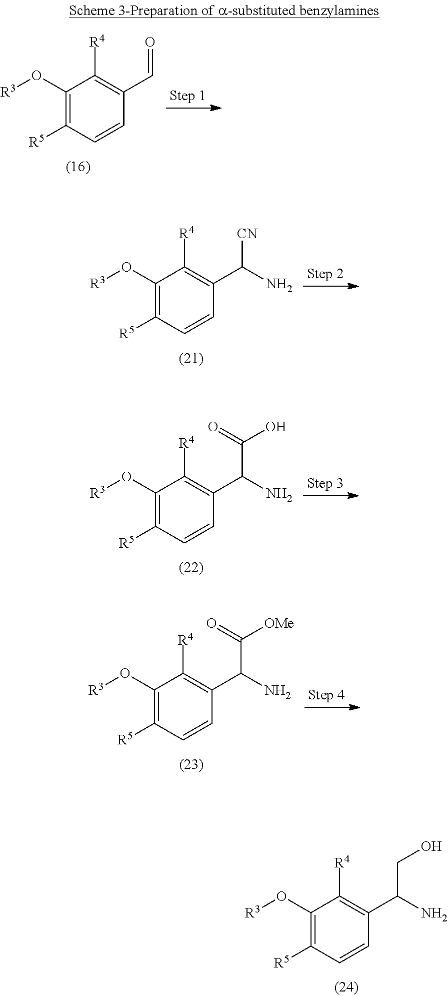

Another route for obtaining alpha-substituted benzylamines from the benzaldehyde (16) is shown in Scheme 3. In Step 1, lithium hexamethyldisilazide in THF is added to the aldehyde (16) at a low temperature (e.g. −40° C.) this is followed by addition of acetone cyanohydrin at room temperature to give the cyanobenzylamine (21).

In Step 2, the cyanobenzylamine (21) is then hydrolysed by reaction with strong acid (e.g. 6N hydrochloric acid), typically with heating at reflux, to give the carboxylic acid (22) which is then converted in Step 3 to the ester (23) by reaction with thionyl chloride and methanol). The ester (23) is then reduced to the alcohol (24) using a suitable reducing agent such as a hydride reducing agent. A preferred method, used in the preparation of compounds described in the experimental section below, is to carry out the reduction using sodium borohydride in an alcohol (e.g. methanol) solvent at a temperature between 0° C. and room temperature.

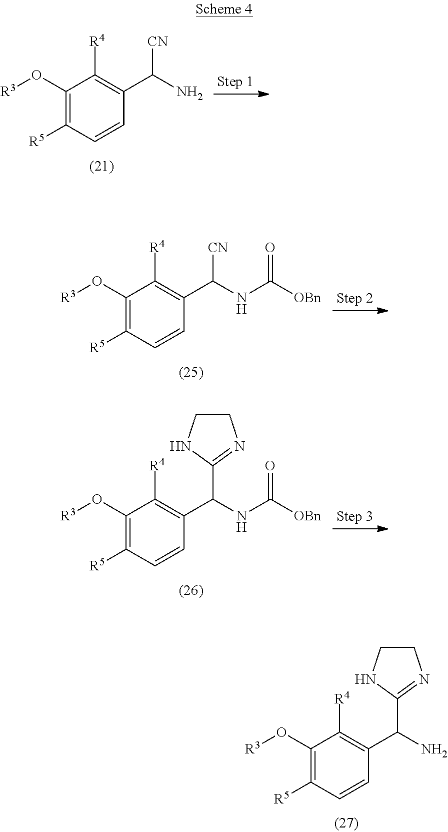

As shown in Scheme 4, the alpha-cyano intermediate (21) generated in Scheme 3 can also be converted into a dihydroimidazole. In Step 1, the primary amino group of the alpha-cyano intermediate (21) is protected, e.g. by conversion to the benzylcarbamate (25) by reaction with benzyl chloroformate in an aqueous organic solvent such as aqueous acetone. The reaction is carried out in the presence of a base such as sodium bicarbonate, typically at approximately room temperature.

The cyano group in the protected amine (25) is then converted to a dihydroimidazole ring in Step 2 by treatment with hydrogen chloride gas in ethanoldiethyl ether solvent at around 0° C. followed by reaction with ethylenendiamine to give the protected dihydroimidazole compound (26) which is then deprotected in Step 3 using hydrogen bromide in acetic acid at a temperature of around 0° C. to give the amine (27).

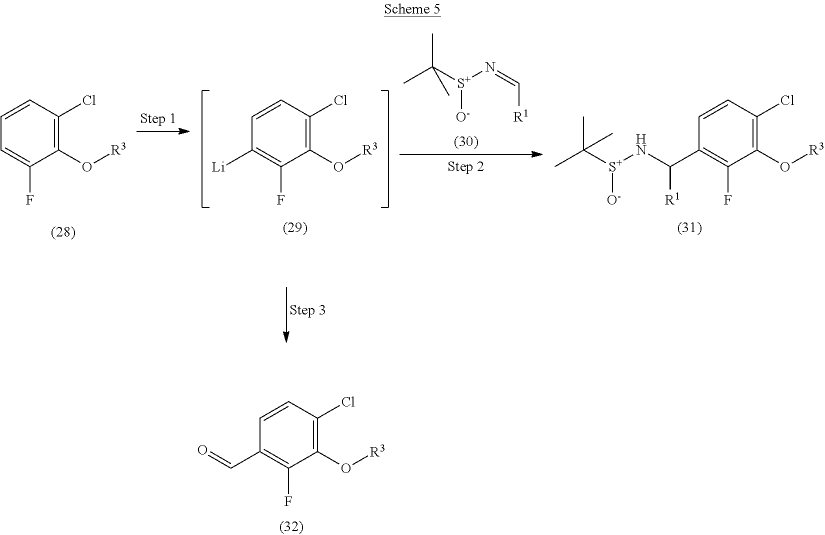

Benzaldehydes of the formula (16) (see Schemes 2 and 3) can also be accessed from intermediates other than a toluene. For example, ortho metallation of a benzene ring followed by formylation can be achieved with the use of a suitable directing group.

In Step 1 of Scheme 5, the example above, the fluorine atom of the fluoro-chloro-phenylether (28) directs lithiation of the phenyl ring to the ortho position. Reaction of the fluoro-chloro-phenylether (28) with a strong lithium base (e.g. sec-butyllithium or tert-butyllithium) in a non-protic solvent (e.g. tetrahydrofuran or diethyl ether) at low temperature (typically below 0° C. and more typically at −78° C.) gives the organolithium intermediate (29). In Step 3, the aldehyde (32) is formed by quenching the organolithium intermediate (29) with dimethylformamide.

Alternatively, as shown in Step 2, the organolithium intermediate (29) can be quenched by addition of the sulphinimide (30) to give the sulphinamide (31) which can be converted to a benzylamine as described in Scheme 2 above. The sulphimimide (30) itself can be obtained by reaction of a compound R¹—CHO with tert-butylsulfinamide in dichloromethane in the presence of a Lewis acid such as titanium tetraethoxide.

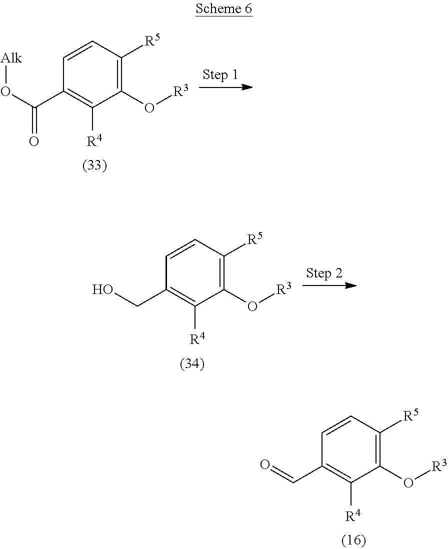

The benzaldehyde precursor (16) to the benzylamine can also be obtained by reduction of a benzoic acid ester followed by oxidation of the resulting alcohol as shown in Scheme 6. Thus, in Step 1, the ester (33) (where Alk is an alkyl group such as ethyl) is reduced to the alcohol (34) using a borane based reducing agent such as borane-tetrahydrofuran complex or an aluminum-based reducing agent such as lithium aluminium hydride in a suitable solvent (e.g. tetrahydrofuran or diethyl ether). In Step 2, the alcohol (34) is oxidised to the aldehyde (16) using an oxidising agent such as manganese (IV) oxide in a chlorinated solvent.

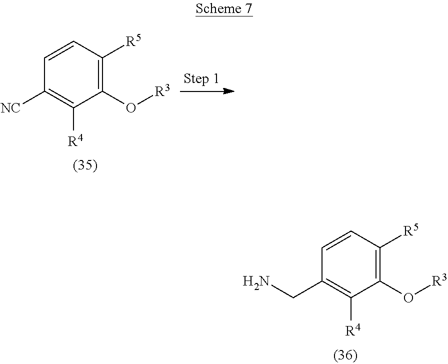

The benzylamine can also be accessed directly from a benzonitrile by reduction with, for example, borane based reducing agents such as borane-tetrahydrofuran complex or aluminium-based reducing agents such as lithium aluminium hydride in a suitable solvent (e.g. tetrahydrofuran or diethyl ether) or by hydrogenation using Raney nickel under a hydrogen atmosphere typically at room temperature and pressure.

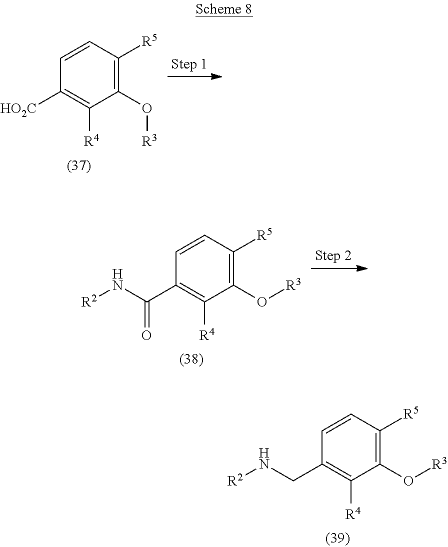

An alternative approach to the benzylamine is by reduction of a benzamide; which in turn can be accessed from a benzoic acid. For example, amide formation from a benzoic acid precursor can be achieved by forming the acyl halide using thionyl chloride or oxalyl chloride in a non-protic solvent or via the mixed anhydride using an alkyl chloroformate in non-protic solvent followed by reaction with a suitable amine. Alternatively this could be achieved using a variety of amide coupling reagents (such as dicyclohexylcarbodiimide and hydroxybenzotriazole). Reduction of the amide to the desired benzylamine can then be achieved using borane based reducing agents such as borane-tetrahydrofuran complex or aluminium-based reducing agents such as lithium aluminium hydride in a suitable solvent (e.g. tetrahydrofuran or diethyl ether).

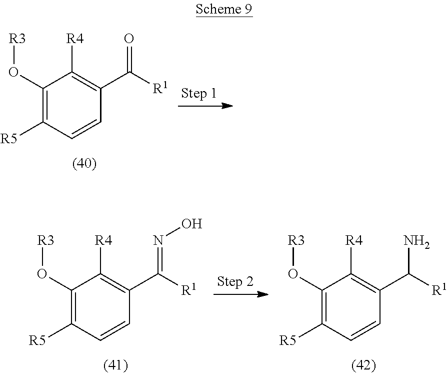

Where suitably substituted ketones are available, they can be converted to the desired benzylamine through oxime formation (e.g. by reaction with hydroxylamine hydrochloride in the presence of sodium acetate) and reduction (e.g. with zinc in acetic acid).

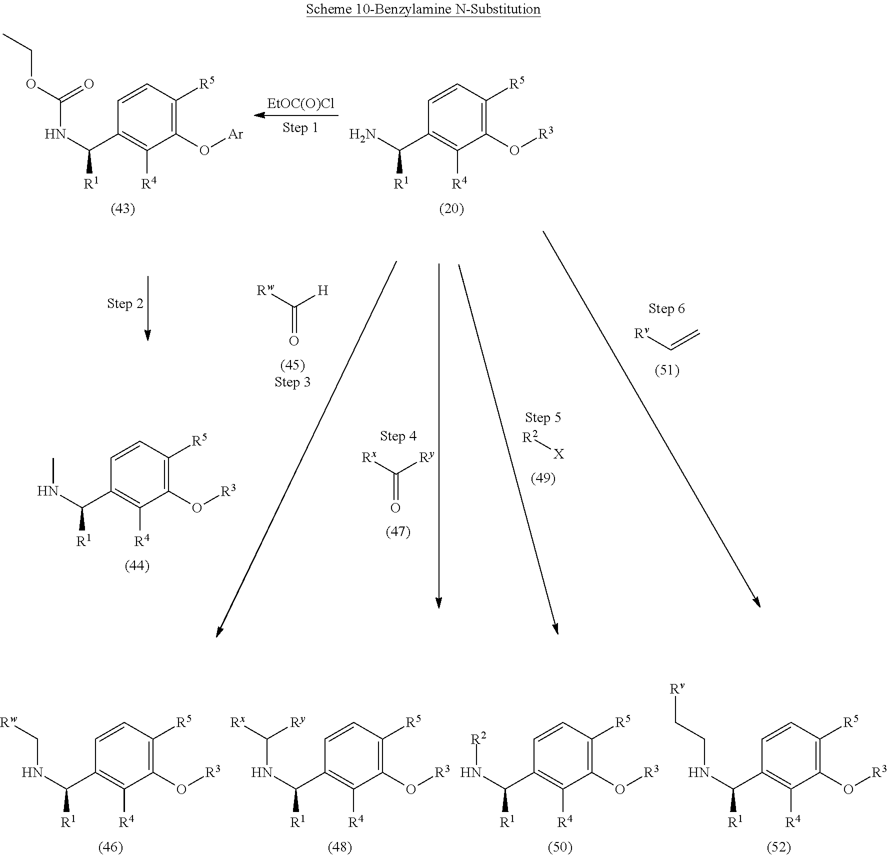

The benzylamine can be further substituted by means of reductive amination (step 3 or 4) whereby an aldehyde or ketone is reacted with the benzylamine and suitable reducing agent such as sodium triacetoxyborohydride in typically tetrahydrofuran or a chlorinated solvent. This procedure can also be achieved in two steps by imine formation under dehydrating conditions where the aldehyde and amine are refluxed (optionally under Dean-Stark conditions) in the presence of catalytic acid (e.g. para-toluenesulfonic acid) or mixed with a Lewis acid in non-protic solvent (e.g. titanium IV chloride or titanium IV isopropoxide in dichloromethane) followed by reduction with suitable reducing agent such as sodium borohydride. Where R^{x} and R^{y} are different and are other than hydrogen, reduction of the imine will give rise to a compound containing a chiral centre at the carbon atom linking R^{x} and R^{y}. By carrying out the reduction under chiral reduction conditions such as chiral hydrogenation, individual optical isomers may be formed preferentially or selectively. For example, chiral hydrogenation of an imine may be carried out using a ruthenium diamine asymmetric catalyst available from Johnson Matthey of Royston, UK.

Alkylation of the amine (step 5) using a compound of the formula R²—X where X is a leaving group such as halogen, triflate or mesylate can be achieved by heating in a suitable solvent or using basic conditions (e.g. alkali metal carbonate in dimethylformamide or dimethylsulfoxide). Arylation or hetetoarylation can be achieved using similar conditions with a suitably electrophilic aryl or heteroaryl halide (e.g. 4-fluoropyridine). Alternatively, an aryl or heteroaryl halide, triflate might be coupled to the benzylamine by transition metal-catalysed coupling (i.e. Buchwald coupling). Michael addition of the amine (step 6) to an activated alkene moiety (e.g. alkyl crotonate) can be achieved at elevated temperatures typically performed neat or with high boiling solvent such as dimethylformamide or N-methylpyrrolidine. Formation of carbamates (step 1) can be achieved using a suitably substituted chloroformate. Reduction of carbamates (step 2) to form the mono-methylamines is possible using lithium aluminium hydride or alternative reductant. Amides can be formed by reaction of a carboxylic acid using amide coupling reagents (such as hydroxybenzotriazole and 1-ethyl-3-(3-dimethylaminopropyl)carbodiimide) and these compounds may optionally be reduced to form alkyl amines (e.g. using lithium aluminium hydride).

### Further Modifications

A number of simple functional group modifications can be made to products and intermediates described above to furnish additional compounds within the scope. Some such transformations are listed in this section; however someone skilled in the art will be able to envisage similar useful transformations.

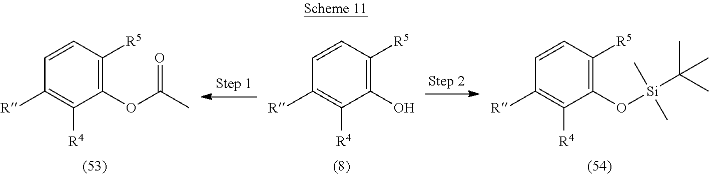

When the phenolic intermediate (8) requires protection, this can be achieved using any one of a number of groups: —see Protective Groups in Organic Synthesis, Greene and Wuts, Wiley Interscience, third edition. Step 2 in Scheme 11 above illustrates the introduction of a tert-butyldimethylsilyl protecting group. This can be accomplished by reacting the compound of formula (8) with the tert-butyldimethylsilyl chloride in the presence of a base (e.g. imidazole) in dimethylformamide. Alternatively, as shown in Step 1, the phenolic hydroxyl group can be protected as an acetyl ester. The acetyl ester can be formed by reacting the compound (8) with acetic anhydride or acetyl chloride in the presence of a base (e.g. triethylamine, pyridine) in a non-protic solvent.

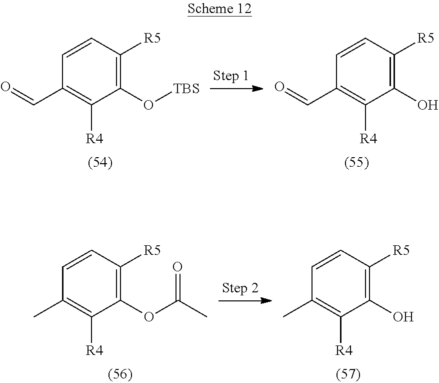

Removal of silyl and acetyl protecting groups from the phenolic hydroxyl group can be accomplished in a number of ways. For example, in order to remove a silyl protecting group as illustrated in Step 1 of Scheme 12, a fluoride source such as tetrabutylammonium fluoride in a non-protic solvent such as tetrahydrofuran can be used. In order to remove an acetyl protecting group, as shown in Step 2, hydrolysis under basic conditions can be employed, for example using an alkali metal hydroxide such as sodium hydroxide in suitable organic solvent such as an alcohol.

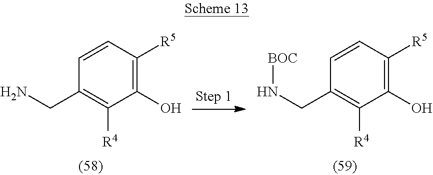

Protection of the benzylamine nitrogen was generally conducted using di tert-butyl dicarbonate in the presence of base such as triethylamine or diisopropylethylamine in ethereal or chlorinated solvent. Intermediate 59 can be further substituted by alkylation. For example, an allyl group can be added by generation of the carbamate anion using sodium hydride and reaction with allyl bromide.

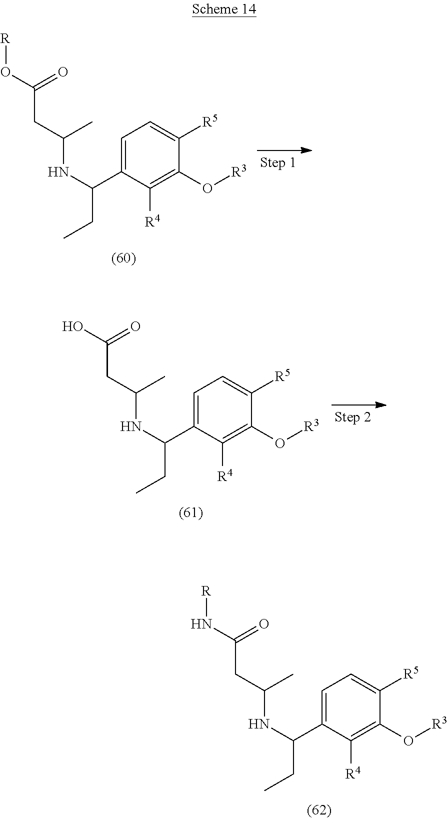

Products of the reaction of relevant benzylamine with a crotonyl ester can be further modified. In the example above, standard modifications known to those skilled in the art are used to convert the terminal ester moiety into an optionally substituted amide. Specifically, a methyl or ethyl ester can be hydrolysed under basic conditions (e.g. aqueous alkali metal hydroxide such as lithium hydroxide in organic solvent such as methanol). A tert-butyl ester can be hydrolysed under acidic conditions (e.g. hydrohalic acid). The resultant acid can be converted to the corresponding amide by reaction with a suitable amine in the presence of a variety of amide coupling reagents (such as dicyclohexylcarbodiimide and hydroxybenzotriazole) in a polar solvent such as dimethylformamide.

Products from biaryl ether formation can be further modified.

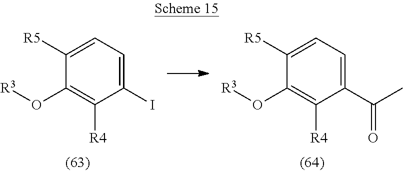

In the example above, standard modifications known to those skilled in the art are used to convert the aryl iodide into the corresponding ketone. Particularly, coupling with tributyl-(1-ethoxyvinyl)-tin under microwave irradiation can be achieved with an appropriate palladium source such as tetrakis(triphenylphosphine)palladium (0) in the presence of lithium chloride and in a suitably polar non-protic solvent such as acetonitrile. The ketone can subsequently be revealed upon treatment with hydrohalic acid.

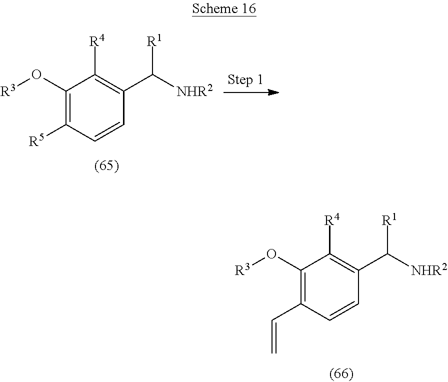

Conversion of an aryl halide such as aryl chloride (e.g. at R⁵) to another group can be conducted. For example, transition metal cross couplings (e.g. Suzuki, Negishi, Buchwald or Heck coupling) can be employed to add a range of carbon, oxygen or nitrogen-linked substituents. In the example above, conversion to a vinyl substituent was achieved using a palladium-mediated coupling with potassium vinyltrifluoroborate. These intermediates can be subjected to further functional group interconversions. For example, the vinyl substituent may be reduced by catalytic hydrogenation.

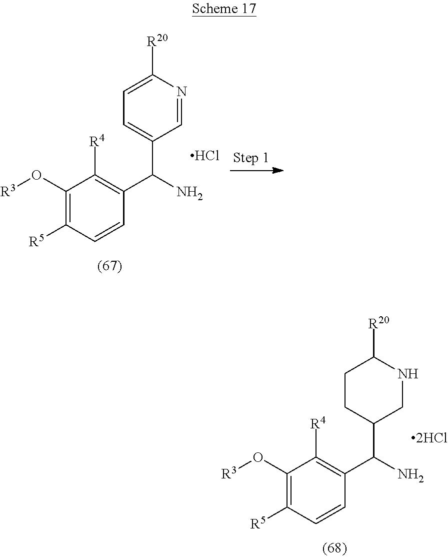

A 3-pyridyl substituted benzylamine can be accessed by addition of 3-pyridyllithium to the sulphinimide followed by deprotection as described above. This intermediate can be converted to the saturated ring by reduction. Typically this would be performed using catalytic hydrogenation using, for example, platinum oxide as catalyst. Where a 2-halo pyridine is formed, it can be converted to the 1H-pyridin-2-one by reaction with strong acids such as 6N hydrochloric acid. Similarly, this intermediate can be reduced according to the method described above.

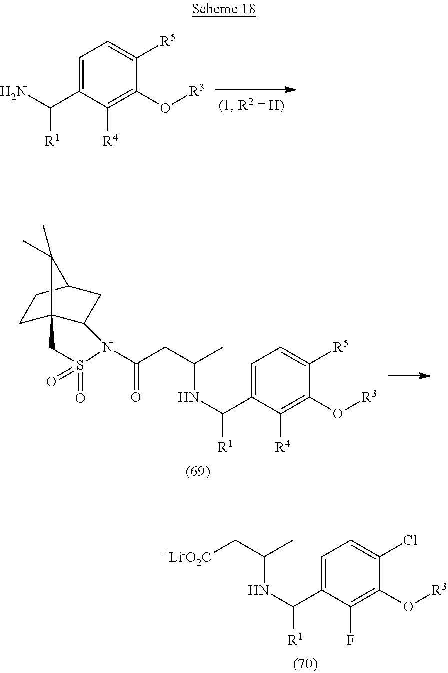

A variation of the approach illustrated in Scheme 14 above is shown in Scheme 18. In Scheme 18, a benzylamine compound of the formula (1) wherein R² is hydrogen is reacted with (R)-(−)-(2-butenoyl)-2,10-camphorsultam in the presence of lithium perchlorate in THF to give the camphorsultam derivative (69). Hydrolysis of the camphorsultam compound with using lithium hydroxide in THF gives the lithium carboxylate salt (70) which can be converted to the carboxylic acid and then to a compound of formula (1) wherein R² is —CH(CH₃)—CH₂—CONHR¹¹ by reaction with an amine of the formula HNHR¹¹ under amide forming conditions of the type described above, for example in the presence of HATU and triethylamine.

The starting materials for the syntheses set out in Schemes 1 to 18 above can be obtained commercially or by using standard synthetic methods well known to the skilled person or analogous thereto, see for example Advanced Organic Chemistry by Jerry March, 4^{th} Edition, John Wiley & Sons, 1992, and Organic Syntheses, Volumes 1-8, John Wiley, edited by Jeremiah P. Freeman (ISBN: 0-471-31192-8), 1995, and see also the methods described in the experimental section below.

Once formed, one compound of the formula (1), or a protected derivative thereof, can be converted into another compound of the formula (1) by methods well known to the skilled person. Examples of synthetic procedures for converting one functional group into another functional group are set out in standard texts such as Advanced Organic Chemistry and Organic Syntheses (see references above) or Fiesers' Reagents for Organic Synthesis, Volumes 1-17, John Wiley, edited by Mary Fieser (ISBN: 0-471-58283-2).

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

### Methods of Purification

The compounds of the invention may be isolated and purified by a number of methods well known to those skilled in the art and examples of such methods include chromatographic techniques such as column chromatography (e.g. flash chromatography) and HPLC. Preparative LC-MS is a standard and effective method used for the purification of small organic molecules such as the compounds described herein. The methods for the liquid chromatography (LC) and mass spectrometry (MS) may be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LCMS; J Comb Chem.; 2004; 6(2), 159-64 and Leister W, Strauss K, Wisnoski D, Zhao Z, Lindsley C., Development of a custom high-throughput preparative liquid chromatographymass spectrometer platform for the preparative purification and analytical analysis of compound libraries; J Comb Chem.; 2003; 5(3); 322-9.

Alternatively, normal phase preparative LC based methods might be used in place of reverse phase methods. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above may alternatively be used to purify the compounds.

Where products or intermediates are chiral, individual optical isomers may be separated by methods well know to the skilled person, for example by:
(i) chiral chromatography (chromatography on a chiral support); or
(ii) forming a salt with an optically pure chiral acid, separating the salts of the two diastereoisomers by fractional crystallisation and then releasing the active compound from the salt; or
(iii) forming a derivative (such as an ester) with an optically pure chiral derivatising agent (e.g. esterifying agent), separating the resulting epimers (e.g. by chromatography) and then converting the derivative to the compound of formula (1).

### Intermediates

Many of the synthetic intermediates described above are themselves novel and, as such, form part of the present application. Accordingly, in a further embodiment (Embodiment 2.1) of the invention, there is provided:

2.1 An intermediate compound selected from:

(a) a compound of the formula (36):

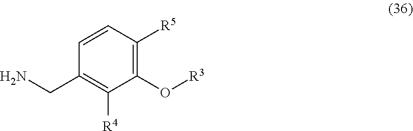

(b) a compound of the formula (21):

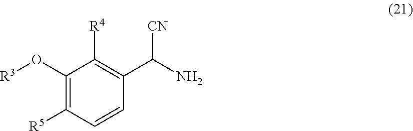

(c) a compound of the formula (23):

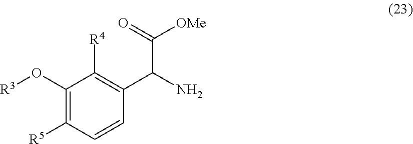

(d) a compound of the formula (22):

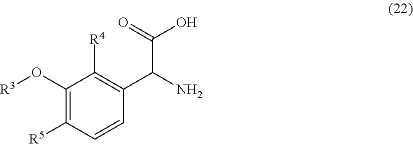

(e) a compound of the formula (23):

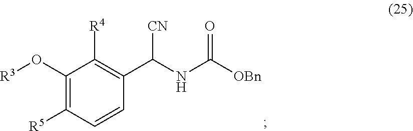

(f) a compound of the formula (17):

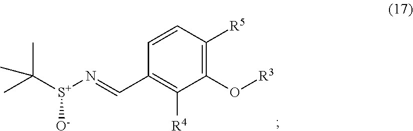

(f) a compound of the formula (18):

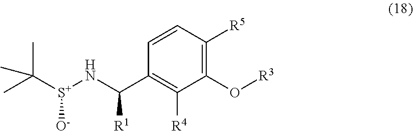

(g) a compound of the formula (19): and

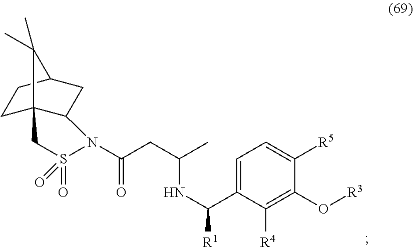

(h) a compound of the formula (20):

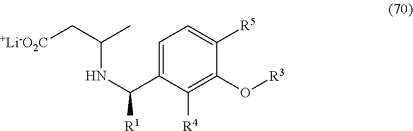

wherein R¹ (where present), R³, R⁴ and R⁵ are as defined in any one of Embodiments 1.1 to 1.112.

Particular intermediates of the invention are the intermediates KI-1 to KI-30 in the experimental section below.

Accordingly, in a further embodiment (Embodiment 2.2), the invention provides a synthetic intermediate selected from the Key Intermediates KI-1 to KI-30 defined herein.

In a further embodiment (Embodiment 2.3), the invention provides a synthetic intermediate selected from the following compounds (19) to (26):

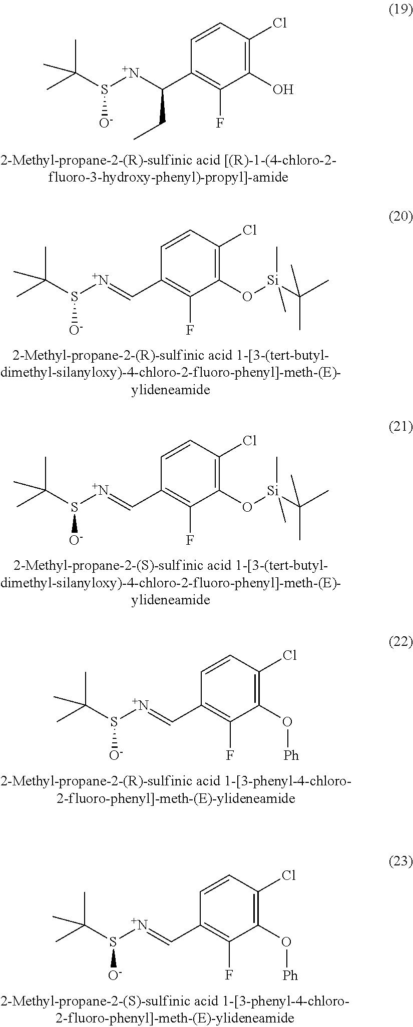

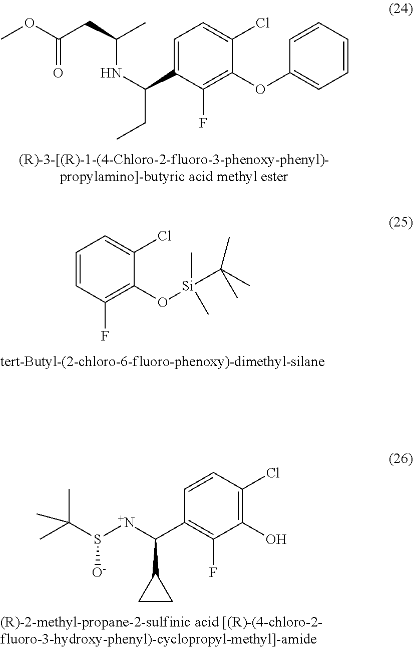

### Biological Activity and Therapeutic Uses

The compounds of Embodiments 1.1 to 1.222 are inhibitors of hepatitis C virus NS3 protease and are therefore beneficial in preventing or treating hepatitis C virus infection and virus-related disorders.

In particular, compounds of Embodiments 1.1 to 1.222 are active against multiple HCV genotypes and resistance mutations.

Compounds of Embodiments 1.1 to 1.222 bind to the allosteric site of the NS3 protein described in Jhoti et al. (idem) and therefore inhibit the function of the NS3 protein. Thus, compounds of the invention are allosteric inhibitors of the NS3 protease helicase

The activity of the compounds can be determined by means of the HCV NS3 protease assay described in Example A andor the replicon assay described in Example B below.

Preferred compounds of the formula (1) are those compounds that have IC₅₀ values of less than 1 μM against the HCV NS3 protease (when determined according to the assay described in Example A (or an assay analogous thereto).

Thus the compounds of the invention may be used for treating or preventing a viral infection or a virus-related disorder in a patient. In particular, such compounds can be inhibitors of HCV replication, and are thus useful for treating viral diseases such as hepatitis C and disorders related to the activity of a virus. In one embodiment, the hepatitis C infection is acute hepatitis C. In another embodiment, the hepatitis C infection is chronic hepatitis C. The compounds can be useful for treating a patient suffering from infection related to particular HCV genotypes as defined herein. HCV types and subtypes may differ in their antigenicity, level of viremia, severity of disease produced, and response to interferon therapy.

The compounds of the invention can also be useful for treating or preventing a disorder related to an HCV infection. Examples of such disorders include, but are not limited to, cirrhosis, portal hypertension, ascites, bone pain, varices, jaundice, hepatic encephalopathy, thyroiditis, porphyria cutanea tarda, cryoglobulinemia, glomerulonephritis, sicca syndrome, thrombocytopenia, lichen planus and diabetes mellitus.

The compounds of the invention may also be used for treating subjects who are suffering from co-infection with HCV and another virus such as hepatitis B (HBV) or human immunodeficiency virus (HIV).

The hypervariability of the HCV genome means that emergence of resistance on treatment with direct-acting antiviral agents (DAAs) is a major problem. Therapeutic intervention with agents acting via several mechanisms is required to increase the barrier to resistance during therapy. The addition of an agent with a new mechanism of action to the treatment regime is therefore an important means of further reducing clinical resistance to therapy. Thus, allosteric inhibitors of protease-helicase represent a new class of therapeutics with the potential for: (i) sensitising HCV to other treatments; (ii) alleviating or reducing the incidence of resistance to DAAs or treatments; (ii) reversing resistance to other DAAs or treatments; (iv) potentiating the activity of other DAAs or treatments; and (v) delaying or preventing the onset of resistance to other DAAs or treatments.

Accordingly, in the further embodiments 3.1 to 3.11 set out below, the invention provides:

3.1 A compound as defined in any one of Embodiments 1.1 to 1.222 wherein the compound has an IC₅₀ value of less than 1 μM against HCV NS3 protease (e.g. when determined according the assays described herein).

3.2 A compound as defined in any one of Embodiments 1.1 to 1.222 wherein the compound has an IC₅₀ value of less than 0.1 μM against HCV NS3 protease (e.g. when determined according the assays described herein).

3.2A A compound as defined in any one of Embodiments 1.0 to 1.329 having inhibitory activity against NS3 helicase.

3.2B A compound as defined in any one of Embodiments 1.0 to 1.329 wherein the compound has an IC₅₀ value of less than 50 μM against HCV NS3 helicase (e.g. when determined according the assays described herein).

3.2C A compound as defined in any one of Embodiments 1.0 to 1.329 wherein the compound has an IC₅₀ value of less than 10 μM against HCV NS3 helicase (e.g. when determined according the assays described herein).

3.2D A compound as defined in any one of Embodiments 1.0 to 1.329 wherein the compound has an IC₅₀ value of less than 5 μM against HCV NS3 helicase (e.g. when determined according the assays described herein).

3.2E A compound as defined in any one of Embodiments 1.0 to 1.329 wherein the compound has an IC₅₀ value of less than 1 μM against HCV NS3 helicase (e.g. when determined according the assays described herein).

3.2F A compound as defined in any one of Embodiments 1.0 to 1.329 wherein the compound has an IC₅₀ value of less than 0.1 μM against HCV NS3 helicase (e.g. when determined according the assays described herein).

3.3 A compound as defined in any one of Embodiments 1.1 to 1.222 for use in medicine or therapy.

3.4 A compound as defined in any one of Embodiments 1.1 to 1.222 for use in the prevention or treatment of hepatitis C virus infections (e.g as defined above).

3.5 A compound as defined in any one of Embodiments 1.1 to 1.222 for use in the treatment of hepatitis C virus infections (e.g. as defined above).

3.6 A compound as defined in any one of Embodiments 1.222 for use in the treatment of hepatitis C virus infection in a subject who has been diagnosed as having hepatitis C virus infection (e.g. as defined above).

3.7 The use of a compound as defined in any one of Embodiments 1.1 to 1.222 for the manufacture of a medicament for the prevention or treatment of hepatitis C virus infections (e.g. as defined above).

3.8 The use of a compound as defined in any one of Embodiments 1.1 to 1.222 for the manufacture of a medicament for the treatment of hepatitis C virus infections (e.g. as defined above).

3.9 The use of a compound as defined in any one of Embodiments 1.1 to 1.222 for the manufacture of a medicament for the treatment of hepatitis C virus infection in a subject who has been diagnosed as having hepatitis C virus infection (e.g. as defined above).

3.10 A method of preventing or treating a hepatitis C virus infection in a subject, which method comprises administering to the subject an effective anti-hepatitis C viral amount of a compound as defined in any one of Embodiments 1.1 to 1.222.

3.11 A method of treating a hepatitis C virus infection in a subject, which method comprises administering to the subject an effective anti-hepatitis C viral amount of a compound as defined in any one of Embodiments 1.1 to 1.222.

3.12 A compound as defined in any one of Embodiments 1.1 to 1.222 for use as an allosteric inhibitor of HCV NS3 protease helicase.

3.13 A method of inhibiting HCV NS3 protease helicase by bringing a compound as defined in any one of Embodiments 1.1 to 1.222 into contact with an allosteric binding site on the NS3 protease helicase.

3.14 A compound as defined in any one of Embodiments 1.1 to 1.222 having a therapeutically useful level of activity as an allosteric inhibitor of the NS3 protease helicase for use in treating hepatitis C viral infections.

3.15 The use of a compound as defined in any one of Embodiments 1.1 to 1.222 having a therapeutically useful level of activity as an allosteric inhibitor of the NS3 protease helicase for the manufacture of a medicament for treating hepatitis C viral infections.

3.16 A compound for use, method or use as defined in any one of Embodiments 3.12 to 3.15 wherein the compound binds to the allosteric binding site described in Jhoti et al., Jhoti et al. Nature Chemical Biology, 2012, doi:10.1038nchembio.1081.

3.17 A compound as defined in any one of Embodiments 1.1 to 1.222 for use in treating a subject (e.g. a mammal such as a human) suffering from hepatitis C(HCV) infection by
(i) sensitising the HCV to other treatments; andor
(ii) alleviating or reducing the incidence of resistance of the HCV to DAAs or treatments; andor
(iii) reversing resistance of the HCV to other DAAs or treatments; andor
(iv) potentiating the activity against the HCV of other DAAs or treatments; andor
(v) delaying or preventing the onset of resistance in the HCV to other DAAs or treatments.

3.18 The use of a compound as defined in any one of Embodiments 1.1 to 1.222 for the manufacture of a medicament for treating a subject (e.g. a mammal such as a human) suffering from hepatitis C(HCV) infection by

(i) sensitising the HCV to other treatments; andor

(ii) alleviating or reducing the incidence of resistance of the HCV to DAAs or treatments; andor

(iii) reversing resistance of the HCV to other DAAs or treatments; andor

(iv) potentiating the activity against the HCV of other DAAs or treatments; andor

(v) delaying or preventing the onset of resistance in the HCV to other DAAs or treatments.

3.19 A method of treating a subject (e.g. a mammal such as a human) suffering from hepatitis C(HCV) infection by:

(i) sensitising the HCV to other treatments; andor

(ii) alleviating or reducing the incidence of resistance of the HCV to DAAs or treatments; andor

(iii) reversing resistance of the HCV to other DAAs or treatments; andor

(iv) potentiating the activity against the HCV of other DAAs or treatments; andor

(v) delaying or preventing the onset of resistance in the HCV to other DAAs or treatments; which method comprises administering to the subject a therapeutically effective amount of a compound as defined in any one of Embodiments 1.1 to 1.222.

3.19A A compound for use, use or method according to any one of Embodiments 3.6, 3.9, 3.10, 3.11 and 3.17 wherein the subject is one who has been co-infected with HCV and another virus such as HBV or HIV.

3.19B A compound for use, use or method according to any one of Embodiments 3.4 to 3.11 and 3.14 to 3.19 wherein the HCV infection is accompanied by infection with another virus such as HBV or HIV.

3.19C A compound, compound for use, use or method according to any one of Embodiments 3.1 to 3.19B wherein the HCV is selected from genotypes 1a, 1b, 2a, 2b, 3a, 4a, 5a and 6a.

3.19D A compound, compound for use, use or method according to any one of Embodiments 3.1 to 3.19B wherein the HCV is selected from genotypes 1a, 1b, 3a, 5a and 6a.

3.19E A compound, compound for use, use or method according to any one of Embodiments 3.1 to 3.19B wherein the HCV is selected from genotypes 1a, 1b and 3a.

The “other DAAs” referred to in Embodiments 3.17 to 3.19 may be any of the therapeutic agents listed in the section headed “Combination Therapy” below and in Embodiments 3.20 and 3.21.

### Posology

The compounds as defined in any one of Embodiments 1.1 to 1.222 are generally administered to a human subject in need of such administration. The human subject will typically have been subjected to tests prior to treatment to establish whether a hepatitis C virus infection is present. The methods of diagnosing the hepatitis C virus infection (e.g. as defined above) may be standard methods well known to the skilled person.

The compounds of the invention will be administered in an effective amount, i.e. an amount which is effective to bring about the desired therapeutic effect

The amount of compound of the invention administered to the subject will depend on the nature of the viral infection and on the characteristics of the subject, such as general health, age, sex, body weight and tolerance to drugs. The skilled person will be able to determine appropriate dosages depending on these and other factors. Effective dosages for commonly used antiviral drugs are well known to the skilled person.

For example, a daily dose of the compound of formula (1) may be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (1) may be administered on a daily basis or on a repeat basis every 2, or 3, or 4, or 5, or 6, or 7, or 10 or 14, or 21, or 28 days for example.

The compounds of the invention may be administered orally in a range of doses, for example 1 to 1500 mg (0.6 to 938 mgm²), or 2 to 800 mg (1.25 to 500 mgm²), or 5 to 500 mg (3.1 to 312 mgm²), or 2 to 200 mg (1.25 to 125 mgm²) or 10 to 1000 mg (6.25 to 625 mgm²), particular examples of doses including 10 mg (6.25 mgm²), 20 mg (12.5 mgm²), 50 mg (31.3 mgm²), 80 mg (50 mgm²), 100 mg (62.5 mgm²), 200 mg (125 mgm²), 300 mg (187.5 mgm²), 400 mg (250 mgm²), 500 mg (312.5 mgm²), 600 mg (375 mgm²), 700 mg (437.5 mgm²), 800 mg (500 mgm²), 900 mg (562.5 mgm²) and 1000 mg (625 mgm²). The compound may be administered once or more than once each day. The compound is typically administered continuously (i.e. taken every day without a break for the duration of the treatment regimen).

In certain circumstances, for example, when used in combination with an anti-cancer drug for treating hepatocellular carcinoma, the compound can be administered continuously or intermittently (i.e. taken continuously for a given period such as a week, then discontinued for a period such as a week and then taken continuously for another period such as a week and so on throughout the duration of the treatment regimen). More usually, the compound of formula (O) will be administered continuously.

Ultimately, however, the quantity of compound administered and the length of the treatment regimen will be at the discretion of a supervising physician.

### Combination Therapy

The compounds of Embodiments 1.1 to 1.222 may be used alone or in combination with other therapeutic agents.

Accordingly, in another embodiment (Embodiment 3.20), the invention provides a combination of a compound as defined in any one of Embodiments 1.1 to 1.222 with at least one (e.g. 1, 2, 3 or 4, or more preferably 1, 2 or 3, and most preferably 2 to 3) other therapeutic agents selected from (a) interferons; (b) ribavirin and analogues thereof; (c) other HCV NS3 protease inhibitors; (d) alpha-glucosidase 1 inhibitors; (e) hepatoprotectants; (f) nucleoside or nucleotide inhibitors of HCV NS5B polymerase; (g) non-nucleoside inhibitors of HCV NS5B polymerase; (h) HCV NS5A inhibitors; (i) TLR-7 agonists; (j) cyclophillin inhibitors; (k) HCV IRES inhibitors; (l) pharmacokinetic enhancers; (m) immunoglobulins; (n) immunomodulators; (O) anti-inflammatory agents; (p) antibiotics; (q) HCV NS3 helicase inhibitors; (r) HCV NS4a antagonists; (s) HCV NS4b binding inhibitors; (t) HCV p7 inhibitors; (u) HCV core inhibitors; and (v) HCV entry inhibitors; (w) diacylglycerol acyltransferase type 1 inhibitors (DGAT-1).

Within Embodiment 3.20, examples of other therapeutic agents are as follows:

Examples of interferons are pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-nl (Wellferon), interferon alpha-n3 (Alferon), Interferon alpha 5 (Digna), injectable HDV-interferon, omega interferon (Intarcia), interferon-beta (Avonex, DL-8234), interferon-omega (omega DUROS, Biomed 510), Zalbin (Albuferon, albinterferon alpha-2b), IFN alpha-2b XL, BLX-883 (Locteron), DA-3021, glycosylated interferon alpha-2b (AVI-005), PEG-[iota]nfergen, PEGylated interferon lambda-1 (PEGylated IL-29) and belerofon.

Examples of ribavirin and its analogues include ribavirin per se (Rebetol, Copegus) and taribavirin (Viramidine).

Examples of HCV NS3 protease inhibitors are boceprevir (SCH-503034), telaprevir (VX-950), TMC-435, BI-201335, Vaniprevir (MK-7009), VX-500, VX-985, VX-813, BMS-650032, GS-9451, GS-9256, MK-5172, ACH-1625, ACH-2684, PHX-1766, Danoprevir (ITMN-191R7227), IDX-320, ABT-450, AVL-181, TG2349, AVL-192.

Examples of alpha-glucosidase 1 inhibitors celgosivir (MX-3253) and Miglitol, UT-231 B.

Examples of hepatoprotectants are IDN-6556, ME 3738, LB-84451, silibilin, MitoQ.

Examples of nucleoside or nucleotide inhibitors of HCV NS5B polymerase are R7128 (RO5024048), IDX-184, BCX-4678, PSI-7977, PSI-938, TMC649128, INX-189, BMS-791325, PSI 353661, ALS2200, ALS2158, GS6620.

Examples of non-nucleoside inhibitors of HCV NS5B polymerase Filibuvir (PF-868554), VX-759, VX-222, BI207127, Tegobuvir (GS-9190), IDX-375, Setrobuvir (ANA-598, VCH-916, MK-3281, VBY-708, A848837, ABT-333, A-48547, VCH-796 (nesbuvir), GSK625433, ABT 072, GS9669, TMC647055.

Examples of HCV NS5A inhibitors Daclastavir (BMS790052), BMS-824393, AZD-7295, AZD-2836 (A-831), EDP-239, PPI-461, PPI-1301, PPI668, ACH 2928, ACH3102, GS5885, GSK2336805, IDX719.

Examples of TLR-7 agonists are ANA-975, ANA-773 and SM-360320.

Examples of cyclophillin inhibitors are Alisporivir (DEBIO-025), SCY-635 and NIM811.

An example of an HCV IRES inhibitor is MCI-067.

An example of an HCV NS4a antagonist is ACH-1095.

An example of an HCV NS4b binding inhibitor is clemizole (Eiger).

Examples of pharmacokinetic enhancers are BAS-100, SPI-452, PF-4194477, TMC-41629 and roxythromycin.

Examples of immunostimulants include Zadaxin (SciClone).

Examples of HCV entry inhibitors are Pro-206, ITX-5061, SP-30.

An example of an HCV p7 inhibitor is BIT-225.

An example of a DGAT-1 inhibitor is LCQ908.

Examples of other drugs used for treating HCV and which may be combined with the compounds of Embodiments 1.0, 1.00 and 1.1 to 1.127 include nitazoxanide (Alinea, NTZ), BIVN-401 (virostat), PYN-17 (altirex), KPE02003002, actilon (CPG-10101), KRN-7000, civacir, GI-5005, XTL-6865, PTX-111, ITX2865, TT-033i, ANA 971, NOV-205, tarvacin, EHC-18, VGX-410C, EMZ-702, AVI 4065, Bavituximab, MDX-1106 (ONO-4538), Oglufanide and VX-497 (merimepodib), SCV-07, Lenocta, CTS-1027, JKB-122, CF-102, PYN17, PYN18, IMMU-105, CYT-107, GSK-2336805, GSK-2485852.

In a further embodiment (Embodiment 3.21), the invention provides a combination of a compound as defined in any one of Embodiments 1.0 to 1.222 with at least one (e.g. 1, 2, 3 or 4, or more preferably 1, 2 or 3, and most preferably 2 to 3) other therapeutic agents selected from (a) interferons; (b) ribavirin and analogues thereof; (c) other HCV NS3 protease inhibitors; (d) alpha-glucosidase 1 inhibitors; (e) hepatoprotectants; (f) nucleoside or nucleotide inhibitors of HCV NS5B polymerase; (g) non-nucleoside inhibitors of HCV NS5B polymerase; (h) HCV NS5A inhibitors; (i) TLR-7 or TLR-9 agonists; (j) cyclophillin inhibitors; (k) HCV IRES inhibitors; (l) pharmacokinetic enhancers; (m) immunoglobulins; (n) immunomodulators; (O) anti-inflammatory agents; (p) antibiotics; (q) HCV NS3 helicase inhibitors; (r) HCV NS4a antagonists; (s) HCV NS4b binding inhibitors; (t) HCV p7 inhibitors; (u) HCV core inhibitors; and (v) HCV entry inhibitors; (w) diacylglycerol acyltransferase type 1 inhibitors (DGAT-1); (x) TLR-3 agonist vaccine adjuvants; (y) viral assembly inhibitors; (z) HIV inhibitors; (aa) viral serine protease inhibitors; (ab) viral polymerase inhibitors; (ac) viral helicase inhibitors; (ad) immunomodulating agents; (ae) antioxidants; (af) antibacterial agents; (ag) therapeutic vaccines; (ah) hepatoprotectant agents; (ai) antisense agents; and (aj) internal ribosome entry site inhibitors.

Within Embodiment 3.21, examples of other therapeutic agents are as follows:

Examples of interferons are pegylated rIFN-alpha 2b (PEG-Intron, Redipen, Sylatron, C-Pegferon, Cylatron, SCH-054031, PEG-IFN-alfa2b, Peginterferon alfa-2b, Virtron, SCH-54031, ViraferonPeg), pegylated rIFN-alpha 2a (Pegasys), rIFN-alpha 2b (Intron A, IFN-alpha2b, YM-14090, Depolnterferon alpha, Alfratronol; Viraferon, Sch-30500), BIP-48 (Peginterferon alfa 2b 48 kDa), rIFN-alpha 2a (Roferon-A, Canferon A, Alphaferon, Interferon alfa-2a, Ro-22-8181, Roceron-A), interferon alpha (Omniferon, Alfanative, Multiferon), YPEG-IFN-alfa2a (Y-peginterferon alfa-2a) interferon alfacon-1 (Infergen, Advaferon, Inferax), interferon alpha-nl (Wellferon, Sumiferon, Sumiferon MP), interferon alpha 2b (Hanferon, SC Interferon-alpha, HL-143), peg Inerferon alpha 2b (P-1101), InferoXen, interferon alpha-n3 (Alferon Naturaferon, Alferon LDO, Human leukocyte interferon alpha, Alferon N Gel, Cellferon, Altemol, Alferon N Injection), Interferon alpha 5 (NAHE-001), injectable HDV-interferon, omega interferon (Intarcia), interferon-beta (Avonex, DL-8234, rHuIFN-beta, Fibroblast interferon, IFN-beta, DL-8234, R-Frone, Feron, Frone), PEG-interferon beta (PEGylated interferon beta, TRK-560) interferon-omega (omega DUROS, Biomed 510), Interferon beta-1a (Rebif, IFN-beta1a, IFN-B-1a) Interferon gamma-1b (Actimmune, Imukin 1, Immukin, DasKloster-1001-01, DasKloster-1001), IFN alpha-2b XL, BLX-883 (Locteron, CR2b), DA-3021, glycosylated interferon alpha-2b (AVI-005), PEG-[iota]nfergen, PEGylated interferon lambda-1 (PEGylated IL-29, BMS-914143, PEG-rIL-29, PEG-Interleukin-29), belerofon, LAPS-IFN alpha (HM-10660A), Alfaferone (Interferon alpha lozenges, BALL-1 IFN-alpha, Natural human lymphoblastoid interferon alfa, Veldona, OPC-18), BBT-012, and Peginterferon alfa-2bribavirin (Pegetron).

Examples of ribavirin and its analogues include ribavirin per se (Rebetol, Copegus, C-Virin; Ravanex, Virazide, Virazole, Ribacine, Cotronak, Viramid) and taribavirin (KD-024, AVS-206, Taribavirin hydrochloride, Viramidine hydrochloride, ICN-3142, Ribamidine hydrochloride, AVS-000206, Viramidine).

Examples of HCV NS3 protease inhibitors are boceprevir (SCH-503034, victrelis), telaprevir (VX-950, incivek, incivo), Simeprevir (TMC-435), Faldaprevir (BI-201335), Vaniprevir (MK-7009), VX-985, VX-813, VBY-376, Asunaprevir (BMS-650032), GS-9451, GS-9256 (GS-337152), MK-5172, Sovaprevir (ACH-1625), Neceprevir (ACH-2684), PHX-1766, Danoprevir (ITMN-191R7227), ABT-450, AVL-181, TG2349, AVL-192, Ossirene (PRX-0002AS101, PRX-0001AS101, IVX-Q-101, WAX-120337, AS-101), BL-8030.

Examples of alpha-glucosidase 1 inhibitors celgosivir (VIR-222, MBI-3253, Bucast, MDL-28574, Bu-cast, MX-3253), Brazaves (Zavesca, NB-DNJ, Vevesca, N-Bu-DNJ, N-Butyl-deoxynojirimycin, Miglustat, OGT-918, SC-48334), Miglitol (Diastabol, Glyset, Plumarol, Seibule).

Examples of hepatoprotectants are Emricasan (IDN-6556, PF-03491390, PF-3491390), Nivocasan (LB-84451), silibilin (Siliphos, Silybin-Phytosome, Silipide, Silybin phosphatidylcholine complex, IdB-1016), MitoQ (Mitoubiquinone mesylate, Mitoquinone mesylate), Molixan (BAM-205, NOV-205), Silymarin (Legalon).

Examples of nucleoside or nucleotide inhibitors of HCV NS5B polymerase are Mericitabine (R7128, RO5024048, MCB, R-4048, RG-7128, RO-5024048), IDX-184, IDX-19368, IDX-19370, BCX-5191 BCX-4678, Sofosbuvir (PSI-7977, GS7977), PSI 353661 (PSI-661), ALS2200, ALS2158, GS6620, T-1106).

Examples of non-nucleoside inhibitors of HCV NS5B polymerase Filibuvir (PF-868554), VX-759, Lomibuvir (VX-222, VCH-222), BI207127, Tegobuvir (GS-9190, GS-333126), IDX-375, PPI-383, VLS-732, Setrobuvir (ANA-598, RG-7790), VCH-916, MK-3281, A848837, ABT-333, A-48547, VCH-796 (nesbuvir), GSK625433, GSK-2485852, ABT 072, GS9669, TMC647055, BMS-791325, PPI-383.

Examples of HCV NS5A inhibitors Daclastavir (BMS790052), BMS-824393, AZD-7295, AZD-2836 (A-831), EDP-239, PPI-461, PPI-1301, PPI-668, ABT-267, ACH 2928, ACH3102, GS5885, GSK2336805, IDX719.

Examples of TLR-7 or TLR-9 agonists are ANA-773 (RG-7795), GS-9620, Resiquimod (R-848, VML-600, S-28463), SD-101, ProMune (PF-03512676, CpG B ODN, Agatolimod sodium, Vaxlmmune, CpG ODN 2006, CpG-2006, PF-3512676, CpG-7909), MCT-465.

Examples of cyclophillin inhibitors are Alisporivir (DEBIO-025, UNIL-025, DEB-025), SCY-635, BC556 and NIM811.

An example of an HCV IRES inhibitor is MCI-067.

An example of an HCV NS4a antagonist is ACH-1095 (ACH-0141095, GS-9525)

An example of an HCV NS4b binding inhibitor is clemizole (Reactrol, Klemidox, Histacuran, Allercur, Clemizole hydrochloride, Eiger).

Examples of pharmacokinetic enhancers are Paradisin C (BAS-100), SPI-452, PF-4194477, GS9350 (Gilead) and ritonavir.

Examples of immunostimulants include Zadaxin (Thymalfasin, Thymosin alpha 1, TA-1), and SM-360320.

Examples of HCV entry inhibitors are ITX-5061, ITX-4520, SP-30, HCV1 MAbM (BL-HCV1), E1E2-VLP and HCV E1E2MF59C.1 (E1E2MF59C.1, HCV E1E2MF59).

An example of an HCV p7 inhibitor is BIT-225.

An example of a DGAT-1 inhibitor is Pradigastat (LCQ-908A, LCQ908)

An example of a TLR-3 agonist is Ampligen (Rintatolimod; Atvogen)

Examples of other drugs used for treating HCV and which may be combined with the compounds of Embodiments 1.1 to 1.222 include nitazoxanide (PH-5776, Heliton, Cryptaz, Colufase, Daxon, Alinea, NTZ), PYN-17 (altirex), KPE02003002, KRN-7000, civacir, GI-5005, ITX2865, TT-033i (OBP-701, TT-033), ANA 971, NOV-205, EHC-18, VGX-410C, EMZ-702, Tarvacin (Bavituximab, Ch3G4), Nivolumab (BMS-936558, MDX-1106, ONO-4538), Oglufanide and VX-497 (merimepodib), Golotide (Golotimod, SCV-07), Lenocta, CTS-1027, JKB-122, CF-102 (CI-IB-MECA), PYN18, IMMU-105, CYT-107, EPB-415, EPB-500, EPB-200, BL-8020, UT-231 B, Nivocasan (GS9450), MK-8742, MK-2748, RO-5466731, RO-5428029, BMS-929075, CH-6808755, JNJ-47910382, VL-01, Vacc-HCV, HS-HIVSIV, TT-034 (PF-05095808), PHN-121, HCV-003 (AdCh3NSmutMVA-NSmut), MK-6325, MG-1105, RO-5303253, SB-9200, PerCvax (Ad6NSmutAdCh3NSmut), TerCvax (AdCh3NSmutAd6NSmut), IPH-1201, REP-2055 (REP-9AC), V-5 Immunitor,), Miravirsen (LNA-anti-mRNA-122, SPC-3649, LNA-antimiR-122), HepTide, PF-4136309 (INCB-8761), Pidilizumab (CT-011), (−)-Epicatechin gallate (ECG, (−)-Epicatechin-3-gallate), CYT-107 (CYT-99-007, rhIL-7, Recombinant interleukin-7), ChronVac-C, KPE-00001133, TG-4040 (MVA-HCV), Nurelin (ADS-5102, ADA; ADS-5101, EXP-105-1, Adamantamine hydrochloride, Lysovir, Mantadix, Hofcomant, Cerebramed, Amantadine hydrochloride, NSC-83653, Symmetrel), Teavigo (Sunphenon, Epigallocatechin-3-gallate, (−)-Epigallocatechin gallate, (−)-EGCG, Epigallocatechin gallate), Prevascar (Ilodecakin, Interleukin-10, IL-10, Tenovil, Sch-52000, rIL-10, rhIL-10), Oxocebron (Ryoxon, WF10, Ancloximex, Oxilium, Oxoferin, Oxoviron, Immunokine, Animexan, Oxomexan, Oxovasin, Oxovir, Macrokine, TCDO, WF-10), Thymogen (IM-862, Oglufanide disodium, Glufanide, Timogen), Civacir (Hepatitis C immune globulin (human), Nabi-Civacir), Phosphostim (IPH-1101, BrHPP sodium salt, Bromohydrin pyrophosphate), Transvax™ (IC-41, Peptide Vaccine IC41, hepatitis C vaccine).

In a preferred embodiment (Embodiment 3.21A), the invention provides a combination of a compound as defined in any one of Embodiments 1.1 to 1.222 with another therapeutic agent selected from telaprevir and boceprevir and combinations thereof, optionally with a further therapeutic (e.g. antiviral) agent such as interferon andor ribavarin.

Combinations with Anti-Cancer Agents

One consequence of infection with hepatitis C virus can be the subsequent development of hepatocellular carcinoma. Combinations of compounds of the invention with anti-cancer drugs may be used to treat hepatocellular carcinoma and in particular early stage hepatocarcinoma.

Accordingly, in further embodiments, the invention provides:

3.22 A combination of a compound according to any one of Embodiments 1.1 to 1.222 and an anti-cancer drug, and more particularly an anti-cancer drug effective in treating hepatocellular carcinoma.

3.23 A combination according to Embodiment 3.22 for use in treating hepatocellular carcinoma.

3.24 The use of a combination according to Embodiment 3.23 for the manufacture of a medicament for the treatment of hepatocellular carcinoma.

3.25 A method of treating hepatocellular carcinoma in a subject in need of such treatment, which method comprises administering to the subject a therapeutically effective amount of a combination as defined in Embodiment 3.22.

3.26 A combination, compound for use, use or method according to any one of Embodiments 3.22 to 3.25 wherein the anti-cancer drug is any one or more (e.g. 1, 2 or 3) selected from 131I-metuximab, AEG-35156, alloCIK, ALN-VSP, alpha-fetoprotein cancer vaccine, apatinib mesylate, ARENEGYR (NGR-TNF, NGR-hTNF), avastin, axitinib, AZD-1480, baclofen, bavituximab, (Tarvacin), BCT-100 (PEG-BCT-100), belinostat, bevacizumab, brivanib alaninate, cabozantinib (cabozantinib S-malate, BMS-907351, XL-184), camptothecin, capecitabine, paclitaxel (e.g. cationic lipid complexed paclitaxel nanoparticles), CF-102 (CI-IB-MECA), cisplatin, cixutumumab, CMS-024, CreaVax-HCC, CryoStim, CT-011, curaxin, darinaparsin (Zinapar), dasatinib, dovitinib lactate, doxorubicin, DW-166HC, ENZ-2968 (EZN-2968, SPC-2968), everolimus, EZN-2968 (ENZ-2968; SPC-2968), ficlatuzumab, flavopiridol, foretinib, fotemustine, ganetespib, GC-33 (RG-7686), golvatinib tartrate, GPC3(144-152)IFA, GPC3(298-306)IFA, GWN (ONO-7268MX1), HAP-302 (TH-302), hepacid (Melanocid, Pegylated arginine deiminase 20000), Immuncell-LC, ImmuCyst, kanglaite, KD-018, KD-025, lansoprazole, lenalidomide, lenvatinib mesylate, linifanib, LY-2157299, mapatumumab, MB-07133 (MB-7133), MEDI-573, melphalan, mepacrine (quinacrine), miriplatin, mitomycin, mitoxantrone, MK-2206 (NSC-749607), MS-20, muparfostat, nemorubicin, nimotuzumab, nintedanib, oncolytic HSV, OPB-31121, orantinib, oxiplatin, pidilizumab, pasireotide, PD-0332991, peretinoin, pexastimogene devacirepvec, Poly-ICLC (Hiltonol), provecta (Xantryl, Rose Bengal disodium), ramucirumab, recentin (AZD-2171), refametinib, regorafenib, resminostat, rF-CEA-TRICOMrV-CEA-TRICOM; CEA-TRICOM, Rose Bengal Sodium, SB-31 (SB Injection, deoxypodophyllotoxin), selumetinib (selumetinib sulfate), sirolimus (Rapamune), sorafenib, tamibarotene, tarceva, talaporfin, TB-403 (Anti-P1GF), temsirolimus, thalidomide, thymalfasin, tigatuzumab, tivantinib, TKM-080301 (PLK1-SNALP; TKM-PLK1), TLC-388, TRC-105, trebananib, tremelimumab, TS-1 (combination of tegafur, gimeracil and oteracil), tyroserleutide (L-Tyrosyl-L-seryl-L-leucine), tyroservaltide (Tyroservatide), vargatef, velcade, veliparib hydrochloride, YN-968D1, zinostatin and zybrestat (Combretastatin A-4).

### Pharmaceutical Formulations

While it is possible for the active compound to be administered alone, it is preferable to present it as a pharmaceutical composition (e.g. formulation).

Accordingly, in another embodiment (Embodiment 4.1) of the invention, there is provided a pharmaceutical composition comprising at least one compound of the formula (1) as defined in any one of Embodiments 1.1 to 1.222 together with at least one pharmaceutically acceptable excipient.

The pharmaceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of pharmaceutical compositions are set out in more detail below.

The term “pharmaceutically acceptable” as used herein pertains to compounds, materials, compositions, andor dosage forms which are, within the scope of sound medical judgment, suitable for use in contact with the tissues of a subject (e.g. a human subject) without excessive toxicity, irritation, allergic response, or other problem or complication, commensurate with a reasonable benefitrisk ratio. Each excipient must also be “acceptable” in the sense of being compatible with the other ingredients of the formulation.

Pharmaceutical compositions containing compounds of the formula (1) can be formulated in accordance with known techniques, see for example, Remington's Pharmaceutical Sciences, Mack Publishing Company, Easton, Pa., USA.

The pharmaceutical compositions can be in any form suitable for oral, parenteral, topical, intranasal, intrabronchial, sublingual, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump or syringe driver.

Pharmaceutical formulations adapted for parenteral administration include aqueous and non-aqueous sterile injection solutions which may contain anti-oxidants, buffers, bacteriostats, co-solvents, surface-active agents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

The formulations may be presented in unit-dose or multi-dose containers, for example sealed ampoules, vials and prefilled syringes, and may be stored in a freeze-dried (lyophilised) condition requiring only the addition of the sterile liquid carrier, for example water for injections, immediately prior to use.

The pharmaceutical formulation can be prepared by lyophilising a compound of formula (1), or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethyl-ellulose and suitable mixtures thereof, vegetable oils (such as sunflower oil, safflower oil and corn oil), and injectable organic esters such as ethyl oleate. Proper fluidity can be maintained, for example, by the use of thickening materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like. Prolonged absorption of the injectable pharmaceutical form may be brought about by the inclusion of agents which delay absorption such as aluminum monostearate and gelatin.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Thus, tablet compositions can contain a unit dosage of active compound together with an inert diluent or carrier such as a sugar or sugar alcohol, eg; lactose, sucrose, sorbitol or mannitol; andor a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citratebicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated. Coatings may act either as a protective film (e.g. a polymer, wax or varnish) or as a mechanism for controlling drug release or for aesthetic or identification purposes. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum, duodenum, jejenum or colon.

Instead of, or in addition to, a coating, the drug can be presented in a solid matrix comprising a release controlling agent, for example a release delaying agent which may be adapted to release the compound in a controlled manner in the gastrointestinal tract. Alternatively the drug can be presented in a polymer coating e.g. a polymethacrylate polymer coating, which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. In another alternative, the coating can be designed to disintegrate under microbial action in the gut As a further alternative, the active compound can be formulated in a delivery system that provides osmotic control of the release of the compound. Osmotic release and other delayed release or sustained release formulations (for example formulations based on ion exchange resins) may be prepared in accordance with methods well known to those skilled in the art.

The compound of formula (1) may be formulated with a carrier and administered in the form of nanoparticles, the increased surface area of the nanoparticles assisting their absorption. In addition, nanoparticles offer the possibility of direct penetration into the cell. Nanoparticle drug delivery systems are described in “Nanoparticle Technology for Drug Delivery”, edited by Ram B Gupta and Uday B. Kompella, Informa Healthcare, ISBN 9781574448573, published 13 Mar. 2006. Nanoparticles for drug delivery are also described in J. Control. Release, 2003, 91 (1-2), 167-172, and in Sinha et al., Mol. Cancer. Ther. Aug. 1, (2006) 5, 1909.

The pharmaceutical compositions typically comprise from approximately 1% (ww) to approximately 95%, preferably % (ww) active ingredient and from 99% (ww) to 5% (ww) of a pharmaceutically acceptable excipient or combination of excipients. Preferably, the compositions comprise from approximately 20% (ww) to approximately 90%,% (ww) active ingredient and from 80% (ww) to 10% of a pharmaceutically excipient or combination of excipients. The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, pre-filled syringes, dragees, tablets or capsules.

The pharmaceutically acceptable excipient(s) can be selected according to the desired physical form of the formulation and can, for example, be selected from diluents (e.g solid diluents such as fillers or bulking agents; and liquid diluents such as solvents and co-solvents), disintegrants, buffering agents, lubricants, flow aids, release controlling (e.g. release retarding or delaying polymers or waxes) agents, binders, granulating agents, pigments, plasticizers, antioxidants, preservatives, flavouring agents, taste masking agents, tonicity adjusting agents and coating agents.

The skilled person will have the expertise to select the appropriate amounts of ingredients for use in the formulations. For example tablets and capsules typically contain 0-20% disintegrants, 0-5% lubricants, 0-5% flow aids andor 0-99% (ww) fillersor bulking agents (depending on drug dose). They may also contain 0-10% (ww) polymer binders, 0-5% (ww) antioxidants, 0-5% (ww) pigments. Slow release tablets would in addition contain 0-99% (ww) release-controlling (e.g. delaying) polymers (depending on dose). The film coats of the tablet or capsule typically contain 0-10% (ww) polymers, 0-3% (ww) pigments, andor 0-2% (ww) plasticizers.

Parenteral formulations typically contain 0-20% (ww) buffers, 0-50% (ww) cosolvents, andor 0-99% (ww) Water for Injection (WFI) (depending on dose and if freeze dried). Formulations for intramuscular depots may also contain 0-99% (ww) oils.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into a polymer or waxy matrix that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules, chewable tablets and dispersible or effervescent tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition a capsule can contain a bulking agent, such as lactose or microcrystalline cellulose. A tablet can contain the solid solution blended with at least one disintegrant, a lubricant, a surfactant, a bulking agent and a glidant. A chewable tablet can contain the solid solution blended with a bulking agent, a lubricant, and if desired an additional sweetening agent (such as an artificial sweetener), and suitable flavours. Solid solutions may also be formed by spraying solutions of drug and a suitable polymer onto the surface of inert carriers such as sugar beads (‘non-pareils’). These beads can subsequently be filled into capsules or compressed into tablets.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use and nasal delivery include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound. Solutions of the active compound may also be used for rectal administration.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (1) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

Where the compound of formula (0) or formula (1) is used in combination with another therapeutic agent (such as another antiviral (e.g. anti-HCV) compound as defined above, the active components of the combination can be physically associated or non-physically associated as defined in the “Definitions” section above. Thus, the other therapeutic agent may be formulated separately to the compound of formula (0) or formula (1) or may be formulated together with the compound of formula (0) or formula (1). In one embodiment (Embodiment 4.2), the compound of formula (0) or formula (1) is formulated together with one or more other therapeutic agents.

Accordingly, in another embodiment (Embodiment 4.2) of the invention, there is provided a pharmaceutical composition comprising at least one compound of the formula (0) as defined in any one of Embodiments 1.0 to 1.222 together with at least one other therapeutic agent as defined herein and at least one pharmaceutically acceptable excipient.

The other therapeutic agent or agents can be any one or more of the agents listed under categories (a) to (z) above.

For example, the pharmaceutical compositions may contain 1, 2 or 3 other therapeutic agents, more typically, 1 or 2 other therapeutic agents.

The one or more other therapeutic agents may be intimately mixed with the compound of formula (0) and formulated together to give a homogeneous composition, or they may be presented in discrete sub-units (e.g. granules, layers, beads or minitablets) which are formulated to give a heterogeneous composition.

Thus, the composition may be presented as a multilayer tablet with one layer comprising the compound of formula (0) and optionally one or more other therapeutic agents and one or more further layers each containing one or more other therapeutic agents.

For example, the composition may take the form of a bilayer or trilayer tablet, with one layer containing the compound of formula (0) and the other layer or layers containing other therapeutic agents as hereinbefore defined.

Where tablet contains two or more layers, one or more layers may be provided with a release delaying-coating that delays release of the compound of formula (0) or another therapeutic agent, for example so that it is released at a different time, or at a different rate, or in a different region of the gastrointestinal tract, from other active agents in the composition.

Alternatively, instead of being presented in separate layers, the tablet composition may be formed from compressed granules wherein two or more different types of granule are present, each type of granule containing a different active agent. For example, the tablet may comprise one type of granules containing a compound of formula (0) and one or more further types of granules containing other therapeutic agents.

As an alternative to tablets, the compositions may be presented as capsules. The capsules may contain a solid, semi-solid or liquid filling in which the compound of formula (0) and the other therapeutic agents form a homogeneous mix, or the capsule may contain a filling in which the compound of formula (0) and the other therapeutic agents form a heterogeneous mix. Thus, the capsule may contain two or more different types of granules, beads or minitablets, wherein each type of granule, bead or minitablet contains a different therapeutic agent or combination of therapeutic agents. For example, one type of granule, bead or minitable may contain a compound of formula (0) and one or more further types of granule, bead or minitablet may contain other therapeutic agents. As with the tablet compositions described above, the various different sub-units (e.g. granules, beads of minitablets) may be formulated for release at different times, different rates or in different parts of the gastrointestinal tract.

The combination of active agents may also be presented as a pharmaceutical kit, pharmaceutical pack or patient pack in which the compound of formula (0) and one or more other therapeutic agents are co-packaged or co-presented (e.g. as part of an array of unit doses); optionally together with instructions for their use.

### EXAMPLES

The invention will now be illustrated, but not limited, by reference to the specific embodiments described in the following examples. In the examples, the following abbreviations are used.

### Abbreviations

DCE 1,2-Dichloroethane

DCM Dichloromethane

DMF N,N-Dimethylformamide

DMSO Dimethylsulfoxide

HCl Hydrochloric acid

Hplc High pressure liquid chromatography

Mins. Minutes

MS Mass Spectrometry

NMR Nuclear Magnetic Resonance Spectroscopy

Petrol Petroleum Ether

Sat. Saturated

THF Tetrahydrofuran

### Analytical LC-MS System and Method Description

In the following examples, compounds were characterised by mass spectroscopy using the systems and operating conditions set out below. Where atoms with different isotopes are present and a single mass quoted, the mass quoted for the compound is the monoisotopic mass (i.e. ³⁵Cl; ⁷⁹Br etc.).

### Waters Platform LC-MS System:

HPLC System: Waters 2795

Mass Spec Detector: Micromass Platform LC

PDA Detector: Waters 2996 PDA

Platform MS Conditions:

Capillary voltage: 3.6 kV (3.40 kV on ES negative)

Cone voltage: 30 V

Source Temperature: 120° C.

Scan Range: 125-800 amu

Ionisation Mode: ElectroSpray Positive or

ElectroSpray Negative or

ElectroSpray Positive & Negative

### Waters Fractionlynx LC-MS System:

HPLC System: 2767 autosampler−2525 binary gradient pump

Mass Spec Detector: Waters ZQ

PDA Detector: Waters 2996 PDA

Fractionlynx MS Conditions:

Capillary voltage: 3.5 kV (3.25 kV on ES negative)

Cone voltage: 40 V (25 V on ES negative)

Source Temperature: 120° C.

Scan Range: 125-800 amu

Ionisation Mode: ElectroSpray Positive or

ElectroSpray Negative or

ElectroSpray Positive & Negative

Agilent 1200SL-6140 LC-MS system—RAPID:

HPLC System: Agilent 1200 series SL

Mass Spec Detector: Agilent 6140 single quadrupole

Second Detector Agilent 1200 MWD SL

Agilent MS Conditions:

Capillary voltage: 4000V on ES pos (3500V on ES Neg)

FragmentorGain: 100

Gain: 1

Drying gas flow: 7.0 Lmin

Gas Temperature: 345° C.

Nebuliser Pressure: 35 psig

Scan Range: 125-800 amu

Ionisation Mode ElectroSpray Positive-Negative switching

### Mass Directed Purification LC-MS System

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LCMS; J Comb Chem.; 2004; 6(2), 159-64 and Leister W, Strauss K, Wisnoski D, Zhao Z, Lindsley C., Development of a custom high-throughput preparative liquid chromatographymass spectrometer platform for the preparative purification and analytical analysis of compound libraries; J Comb Chem.; 2003; 5(3); 322-9.

Several systems for purifying compounds via preparative LC-MS are described below although a person skilled in the art will appreciate that alternative systems and methods to those described could be used. From the information provided herein, or employing alternative chromatographic systems, a person skilled in the art could purify the compounds described herein by preparative LC-MS.

### Preparative LC-MS System Description:

### Waters Fractionlynx System:

Hardware:

2767 Dual Loop AutosamplerFraction Collector

2525 preparative pump

CFO (column fluidic organiser) for column selection

RMA (Waters reagent manager) as make up pump

Waters ZQ Mass Spectrometer

Waters 2996 Photo Diode Array Detector

Waters ZQ Mass Spectrometer

Waters MS Running Conditions:

Capillary voltage: 3.5 kV (3.2 kV on ES Negative)

Cone voltage: 25 V

Source Temperature: 120° C.

Scan Range: 125-800 amu

Ionisation Mode: ElectroSpray Positive or

### ElectroSpray Negative

### Agilent 1100 LC-MS Preparative System:

Hardware:

Autosampler: 1100 series “prepALS”

Pump: 1100 series “PrepPump” for preparative flow gradient and 1100 series “QuatPump” for pumping modifier in prep flow

UV detector: 1100 series “MWD” Multi Wavelength Detector

MS detector: 1100 series “LC-MSD VL”

Fraction Collector: 2×“Prep-FC”

Make Up pump: “Waters RMA”

Agilent Active Splitter

Agilent MS Running Conditions:

Capillary voltage: 4000 V (3500 V on ES Negative)

FragmentorGain:1501

Drying gas flow: 12.0 Lmin

Gas Temperature: 350° C.

Nebuliser Pressure: 50 psig

Scan Range: 125-800 amu

Ionisation Mode ElectroSpray Positive or

ElectroSpray Negative

Columns:

A range of commercially available columns—both achiral and chiral—were used such that, in conjunction with the changes in mobile phase, organic modifier and pH, they enabled the greatest cover in terms of a broad range of selectivity. All columns were used in accordance with the manufacturers recommended operating conditions. Typically 5 micron particle sized columns were used where available. For example, columns from Waters (including but not limited to XBridge™ Prep OBD™ C18 and Phenyl, Atlantis® Prep T3 OBD™ and Sunfire™ Prep OBD C18 5 μm 19×100 mm), Phenomenex (including but not limited to Synergy MAX-RP and LUX™ Cellulose-2), Astec (Chirobiotic™ columns including but not limited to V, V2 and T2) and Diacel® (including but not limited to Chiralpak® AD-H) were available for screening.

Eluents:

Mobile phase eluent was chosen in conjunction with column manufacturers recommended stationary phase limitations in order to optimise a columns separation performance.

Methods:

### Achiral Preparative Chromatography

The compound examples described have undergone HPLC purification, where indicated, using methods developed following recommendations as described in Snyder L. R., Dolan J. W., High-Performance Gradient Elution The Practical Application of the Linear-Solvent-Strength Model, Wiley, Hoboken, 2007.

### Chiral Preparative Chromatography

Preparative separations using Chiral Stationary Phases (CSPs) are the natural technique to apply to the resolution of enantiomeric mixtures. Equally, it can be applied to the separation of diastereomers and achiral molecules. Methods are well known in the art for optimising preparative chiral separations on CSPs and then using them to purify compounds. Such methods are described in Beesley T. E., Scott R. P. W.; Chiral Chromatography; Wiley, Chichester, 1998.

### Salt Formation

Target molecules containing a basic centre were routinely converted to the corresponding hydrochloride salt by treatment with for example sat. HCl in EtOAc or 4M HCl in dioxane, followed by evaporation. Trituration with a suitable solvent such as Et₂O and collection by filtration followed by drying under vacuum gave the target molecule as a solid.

### Synthesis of Key Intermediate 1

### (S)-1-(2,4-Difluoro-3-phenoxy-phenyl)-propylamine

### Step 1

A mixture of 2,6-difluoro-3-methylphenol (10.1 g, 70 mmol), phenyl boronic acid (8.6 g, 70 mmol), copper (II) acetate (12.7 g, 70 mmol), pyridine (29 ml, 350 mmol), pyridine N-oxide (7.3 g, 77 mmol) and powdered 4 Å molecular sieves (12.8 g) in DCM (400 ml) was stirred at room temperature overnight. The reaction mixture was filtered and the filtrate concentrated. The residue was partitioned between 2M HCl and petrol. The organic fractions were dried over magnesium sulfate, filtered and concentrated to afford 2,4-difluoro-3-phenoxytoluene (11.34 g, 74%) as a pale yellow liquid. ¹H NMR (400 MHz, CDCl₃): 7.40-7.18 (2H, m), 7.18-7.06 (1H, m), 7.06-6.84 (4H, m), 2.30 (3H, s).

### Step 1—Alternative Procedure

A solution of 2-trimethylsilyloxyphenyl triflate (10 g, 3.4 mmol) in acetonitrile (25 ml) was added dropwise to a solution of 2,6-difluoro-3-methylphenol (490 mg, 3.4 mmol) and cesium fluoride (15.2 g, 10 mmol) in acetonitrile (50 ml) under an inert atmosphere. The resulting suspension was stirred for 3 hours, quenched with 10% potassium hydroxide (100 ml) and extracted into petrol (5×100 ml). The combined organic fractions were dried over magnesium sulfate, filtered and concentrated to afford 2,4-difluoro-3-phenoxytoluene (660 mg) as a pale brown oil.

### Step 2

A solution of 2,4-difluoro-3-phenoxytoluene (21.7 g, 98 mmol), N-bromosuccinimide (21 g, 118 mmol) and azabisisobutyronitrile (217 mg, 1.3 mmol) in carbon tetrachloride (217 ml) was heated at 80° C. under an inert atmosphere overnight. Azabisisobutyronitrile (217 mg, 1.3 mmol) was added and the reaction heated to 90° C. for a further 3 hours. Water (100 ml) was added and the layers separated. The organic phase was washed with water, dried over magnesium sulfate, filtered and concentrated to yield 1-bromomethyl-2,4-difluoro-3-phenoxybenzene (31.96 g) which was used without further purification. ¹H NMR (400 MHz, CDCl₃): 7.36-7.32 (2H, m), 7.11 (2H, q), 7.07-6.99 (1H, m), 6.97 (2H, d), 4.52 (2H, s).

### Step 3

A solution of 1-bromomethyl-2,4-difluoro-3-phenoxybenzene (32 g, 98 mmol) and sodium hydrogen carbonate (50.4 g, 600 mmol) in DMSO (160 ml) was heated at 80° C. under an inert atmosphere overnight. The reaction was partitioned between water and petrol. The organic fractions were dried over magnesium sulfate, filtered and concentrated and the residue purified by distillation under reduced pressure. Heating to 100° C. at 0.2 mbar, the 2,4-difluoro-3-phenoxybenzaldehyde product (21.8 g) was collected as a colourless liquid. ¹H NMR (400 MHz, DMSO-d₆): 10.16 (1H, s), 7.91-7.79 (1H, m), 7.57-7.45 (1H, m), 7.45-7.36 (2H, m), 7.20-7.08 (1H, m), 7.04 (2H, d).

### Steps 2 and 3—Alternative Procedure

A solution of 2,4-difluoro-3-phenoxytoluene (6.5 g, 29.6 mmol), N-bromosuccinimide (15.8 g, 88.7 mmol) and azabisisobutyronitrile (350 mg, 2.1 mmol) in carbon tetrachloride (70 ml) was heated at 80° C. under an inert atmosphere overnight. azabisisobutyronitrile (100 mg, 0.6 mmol) and N-bromosuccinimide (2.5 g, 14.0 mmol) were added and the reaction heated to 80° C. overnight. Water (100 ml) was added and the layers separated. The aqueous phase was extracted with DCM (2×40 ml). The combined organic fractions were washed with water and brine, dried over magnesium sulfate, filtered and concentrated to yield 1-dibromomethyl-2,4-difluoro-3-phenoxybenzene (31.96 g) which was used without further purification.

The 1-dibromomethyl-2,4-difluoro-3-phenoxybenzene was dissolved in iso-propyl alcohol (120 ml) and silver nitrate (10.1 g, 59.2 mmol) was added, followed by water (24 ml). The reaction was stirred at room temperature for 3 hours, then filtered and the solid washed with iso-propyl alcohol.

The filtrate was concentrated, diluted with water (50 ml) and kept in a fumehood overnight before being extracted with DCM (2×50 ml). Organic fractions were dried over magnesium sulfate, filtered and evaporated to dryness. The residue was purified by column chromatography, eluting with 5% ethyl acetate in petrol to yield 2,4-difluoro-3-phenoxybenzaldehyde (7.0 g) as a yellow liquid.

### Step 4

Titanium (IV) ethoxide (1.8 ml, 8.54 mmol) was added to a solution of 2,4-difluoro-3-phenoxybenzaldehyde (1 g, 4.27 mmol) and (S)-tert-butylsulfinimide (520 mg, 4.48 mmol) in DCM (15 ml) under an inert atmosphere and the resulting mixture was stirred overnight. A suspension of sodium sulfate (10 g) in DCM (15 ml) was added and the mixture stirred vigourously for 1 hour before being filtered. The filtrate was evaporated to dryness to give (S)-2-methyl-propane-2-sulfinic acid 1-(2,4-difluoro-3-phenoxy-phenyl)meth-(E)-ylideneamide (1.40 g, 98%) as a white solid. ¹H NMR (400 MHz, DMSO-d₆): 8.64 (1H, s), 8.03-7.93 (1H, m), 7.55-7.45 (1H, m), 7.41-7.36 (2H, m), 7.14 (1H, t), 7.04 (2H, d), 1.21 (9H, s).

### Step 5

Ethyl magnesium bromide (2.8 ml of a 3M solution in THF, 2.4 mmol) was added dropwise to a solution of (S)-2-methyl-propane-2-sulfinic acid 1-(2,4-difluoro-3-phenoxy-phenyl)-meth-(E)-ylideneamide (1.4 g, 4.15 mmol) in THF (28 ml) under an inert atmosphere at −78° C. The reaction was stirred for 2 hours at −78° C. before being quenched with sat. ammonium chloride (15 ml) and allowed to warm to room temperature. The reaction mixture was partitioned between water and ethyl acetate. The organic fractions were washed with brine, dried over magnesium sulfate, filtered and evaporated and the residue purified by column chromatography. Elution with 0-50% ethyl acetate in petrol afforded the desired (S,S) isomer, 2-methyl-propane-2-(S)-sulfinic acid [(S)-1-(2,4-difluoro-3-phenoxy-phenyl)-propyl]amide, (0.92 g, 61%) as a colourless oil. ¹H NMR (400 MHz, DMSO-d₆): 7.54-7.42 (1H, m), 7.41-7.27 (3H, m), 7.11 (1H, t), 6.94 (2H, d), 4.41-4.29 (1H, m), 1.94-1.79 (1H, m), 1.77-1.62 (1H, m), 1.11 (9H, s), 0.86 (3H, t). Further elution yielded the other (R,S) isomer (0.12 g) also as a colourless oil. ¹H NMR (400 MHz, DMSO-d₆): 7.48-7.26 (4H, m), 7.11 (1H, t), 6.91 (2H, d), 4.39 (1H, q), 1.99-1.87 (1H, m), 1.84-1.67 (1H, m), 1.07 (9H, s), 0.85 (3H, t).

### Step 6

2-Methyl-propane-2-(S)-sulfinic acid [(S)-1-(2,4-difluoro-3-phenoxy-phenyl)-propyl]-amide (920 mg, 2.5 mmol) was dissolved in methanol (10 ml) and HCl (2 ml of a 4M solution in dioxane, 8 mmol) was added. The solution was stirred for 1 hour, then concentrated and the residue was triturated with diethyl etherpetrol (1:1) to afford Key Intermediate 1 (596 mg, 90%) as a white solid.

### Synthesis of Key Intermediate 2

### 2-Methyl-propane-2-(S)-sulfinic acid 1-[3-(tert-butyl-dimethyl-silanyloxy)-2,4-difluoro-phenyl]-meth-(E)-ylideneamide

### Step 1

A solution of 2,6-difluorophenol (130 g, 1 mol), tert-butyldimethylsilyl chloride (146 g, 0.97 mol) and imidazole (75 g, 1.1 mol) in DMF (650 ml) was stirred at room temperature overnight before being diluted with water (1.9 L) and extracted into petrol (3×650 ml). Combined organic fractions were washed consecutively with 10% potassium carbonate, water and brine, dried over magnesium sulfate, filtered and evaporated to dryness to give afforded 3-(tert-butyl-dimethyl-silanyloxy)-2,4-difluoro-benzene (226.5 g, 96%) as a colourless liquid.

### Step 2

sec-Butyl lithium (57 ml of a 1.3M solution in THF, 75.8 mmol) was added dropwise to a solution of the product of Step 1 (12 g, 49.2 mmol) in THF (50 ml) at −78° C. over a 45 min period. The solution was stirred for 30 mins more at this temperature before DMF (15 ml) was added. After a further 30 mins, sat. ammonium chloride was added and the reaction mixture was allowed to warm to room temperature before being extracted into ethyl acetate (3×30 ml). The organic fractions were dried over sodium sulfate, filtered, concentrated and subjected to column chromatography. Elution with 2% ethyl acetate in petrol afforded 3-(tert-butyl-dimethyl-silanyloxy)-2,4-difluoro-benzaldehyde (4.3 g, 32%) as a colourless oil. MS: [M+H]⁺273.

### Step 3

3-(tert-Butyl-dimethyl-silanyloxy)-2,4-difluoro-benzaldehyde (4.3 g) from Step 2 was condensed with (S)-tert-butyl sulfinimide as described in Key Intermediate 1, step 4 to generate Key Intermediate 2 (4.34 g) as a colourless oil.

### Synthesis of Key Intermediate 3a

### 2-Methyl-propane-2-(R)-sulfinic acid [(R)-1-(4-chloro-2-fluoro-3-hydroxy-phenyl)-propyl]-amide

### Step 1

To a 5L flange flask fitted with stirrer bar and nitrogen inletoutlet was added 6-chloro-2-fluoro-3-methyl phenol (200 g, 1.245 moles, 1.0 eq), pyridine (352 ml) and acetic anhydride (190.7 g, 177 ml, 1.868 moles, 1.08 eq). The mixture was heated at 50° C. for 60 minutes after which time NMR confirmed the reaction to be complete. The solvent was removed under reduced pressure at 50° C., the residue was diluted with ethyl acetate (1 L) and washed with 0.5M HCl (1 L), the aqueous was re-extracted with ethyl acetate (1 L). The organics were combined, washed with sat sodium hydrogen carbonate (1 L), then brine (1 L), dried over magnesium sulfate, filtered and evaporated to dryness at 40° C. to give acetic acid 6-chloro-2-fluoro-3-methyl-phenyl ester as a straw coloured oil, yield=244 g, 97%.

### Step 2

To a 5L flange flask fitted with stirrer bar, condenser and nitrogen inletoutlet was added acetic acid 6-chloro-2-fluoro-3-methyl-phenyl ester (244 g, 1.20 moles, 1.0 eq), carbon tetrachloride (2.4 L), azabisisobutyronitrile (12.2 g, 0.06 moles, 0.05 eq), N-bromosuccinimde (643 g, 3.61 moles, 3.0 eq). The orange coloured mixture was heated at 80° C. overnight after which time NMR confirmed ˜3% of the mono-bromo compound remaining. Further N-bromosuccinimde (64.3 g, 0.361 moles, 0.3 eq) and azabisisobutyronitrile (6.3 g, 0.03 moles, 0.025 eq) was added and the mixture heated for a further 3 hours. NMR showed approx 1% mono-bromo intermediate left plus other impurities starting to form—mixture worked-up. Water (2 L) was added, the organic layer removed and the aqueous re-extracted with DCM (2 L). The organic extracts were combined, dried over magnesium sulfate, filtered and evaporated to dryness. After 90% of the solvent was removed a solid started to precipitate, this was filtered off and the filtrate evaporated to dryness, NMR showed the solid to not contain product. The crude product was re-dissolved in DCM and evaporated to dryness to remove any residual traces of carbon tetrachloride. This procedure was repeated twice. The desired product, acetic acid 6-chloro-3-dibromomethyl-2-fluoro-phenyl estert was obtained as an orange oilsolid, yield=472 g (yield is over 100%-probably contains some N-bromosuccinimde impurity).

### Step 3

To a 10L flange flask fitted with stirrer bar, temperature probe and dropping funnel was added acetic acid 6-chloro-3-dibromomethyl-2-fluoro-phenyl ester (472 g, assume 1.20 moles, 1.0 eq) in i-propanol (4 L). To the water bath cooled solution was added dropwise over 10 minutes a solution of silver nitrate (408 g, 2.40 moles, 2.0 eq) in Water (800 ml). During addition a cream precipitate formed and the internal temperature rose to 32° C. After addition was complete the mixture was stirred for 1 hour, NMR confirmed the reaction to be complete. The solvent was removed under reduced pressure at 40° C. and the residue suspended in DCM (2 L) and water (2 L), then filtered through cellite. The organic phase was removed and the aqueous re-extracted with DCM (2 L). The organic extracts were combined, dried over magnesium sulfate, filtered and evaporated to dryness at 40° C. to give acetic acid 6-chloro-2-fluoro-3-formyl-phenyl ester as an orange oil, yield=253 g.

### Step 4

To a 3 L flange flask fitted with stirrer bar was added acetic acid 6-chloro-2-fluoro-3-formyl-phenyl ester (253 g, 1.17 moles, 1.0 eq) in methanol (800 ml). To the solution was added 10% sodium hydroxide (800 ml)—an immediate dark coloured solution resulted. The mixture was heated to 50° C., after 60 minutes NMR confirmed the reaction to be complete. The solvent was removed under reduced pressure at 40° C., the residue was diluted with water (1.5 L) and poured into concentrated HCl (300 ml) causing a precipitate to result. This was removed by filtration and dried under vacuum to give the crude product, yield=173 g. The crude material was stirred overnight in 5% ethyl acetatepetrol (1 L) then filtered off and dried to give 4-chloro-2-fluoro-3-hydroxybenzaldehyde as a tan coloured solid, yield=144 g

### Step 5

To a 3L flange flask fitted with stirrer bar and nitrogen inletoutlet was added 4-chloro-2-fluoro-3-hydroxybenzaldehyde (140 g, 0.802 moles, 1.0 eq) followed by DMF (500 ml), tert-butyldimethylsilyl chloride (145 g, 0.96 moles, 1.2 eq) and imidazole (76 g, 1.12 moles, 1.4 eq). The mixture was stirred at room temperature overnight. NMR confirmed the reaction to be complete. The mixture was diluted with water (2 L) and extracted with petrol (2 L), the aqueous was re-extracted with petrol (2 L). The organic extracts were combined, washed with 2M HCl (1 L), then brine (1 L) then dried over magnesium sulfate, filtered and evaporated to dryness at 40° C. to give the crude product as a brown oil, yield=252 g. This material was then purified by suction column chromatography on a 4 L sinter, loaded onto the column in petrol and eluted using ethyl acetatePetrol, 0-6% ethyl acetate, 2% steps, 4 L per step to give 3-tert-butyldimethylsilanyloxy-4-chloro-2-fluorobenzaldehyde as a golden oil, yield=205 g.

### Step 6

To a 10L flange flask fitted with overhead stirrer was added 3-tert-butyldimethylsilanyloxy-4-chloro-2-fluorobenzaldehyde (100 g 0.346 moles, 1.0 eq) followed by DCM (1.5 L), (R)-(+)-2-Methyl-2-propane sulfonamide (44 g, 0.363 moles, 1.05 eq) and finally titanium (IV) ethoxide (160 g, 0.70 moles, 2.0 eq). The straw coloured mixture was stirred at room temperature overnight under nitrogen. After overnight stirring the mixture had darkened and NMR confirmed the reaction to be complete. To the mixture was added DCM (1.5 L) followed by sodium sulphate decahydrate (1.03 Kg). The mixture was stirred vigorously for 1 hour before filtration through cellite—quite slow. The cellite pad was washed well with DCM (5×1 L), the filtrate was evaporated to dryness at 40° C. on rotary then any residual water azeotroped with toluene to give (R)-2-methyl-propane-2-sulfinic acid 1-[3-(tert-butyl-dimethyl-silanyloxy)-4-chloro-2-fluoro-phenyl]-meth-(E)-ylideneamide as a yellow oil, yield=140 g (contains some toluene).

### Step 7

To a 5L Flange flask fitted with stirrer bar, nitrogen inletoutlet and temperature probe was added (R)-2-methyl-propane-2-sulfinic acid 1-[3-(tert-butyl-dimethyl-silanyloxy)-4-chloro-2-fluoro-phenyl]-meth-(E)-ylideneamide (140 g, 0.357 moles, 1.0 eq) followed by THF (2.5 L). The mixture was cooled to −78° C. before cannula addition of EtMgBr (3.0 M in Et₂O, 238 ml, 0.714 moles, 2.0 eq). During addition the mixture goes a milky colour and slightly thicker—still able to stir using stirrer bar. The mixture was stirred for 3 hours after which time NMR confirmed the reaction to be complete. The mixture was quenched by the addition of sat. ammonium chloride (1.25 L). The mixture was extracted with ethyl acetate (2×2 L), dried over magnesium sulfate, filtered and evaporated to dryness to give the crude product as a straw coloured oil, yield=141.5 g. The crude product was dissolved in a small amount of DCM and loaded onto a column (silica bed size 13 cm×24 cm). The product was eluted using Petrolethyl acetate, 0-35%, 2 L per step, 5% steps until 30% & 35%-4 L. The desired product, major enantiomer, (R)-2-methyl-propane-2-sulfinic acid {(R)-1-[3-(tert-butyl-dimethyl-silanyloxy)-4-chloro-2-fluoro-phenyl]-propyl}-amide, was isolated as an off-white solid, yield=76.5 g, the minor enantiomer was isolated as a viscous straw coloured oil, yield 33.8 g, some mixed fractions were also isolated yield=5.5 g.

### Step 8

A mixture of (R)-2-methyl-propane-2-sulfinic acid {(R)-1-[3-(tert-butyl-dimethyl-silanyloxy)-4-chloro-2-fluoro-phenyl]-propyl}-amide (70 g, 0.166 mol) and cesium fluoride (76 g, 0.498 mol) in acetonitrile (700 ml) and water (350 ml) was stirred at room temperature overnight. The reaction was shown to be complete by TLC (1:1 ethyl acetate: petrol). After diluting with brine (350 ml) and diethyl ether (350 ml), the mixture was stirred vigorously before the phases were separated. The aqueous fraction was extracted with diethyl ether (350 ml) and the combined organic liquors were dried (MgSO₄) and concentrated to furnish a white solid. This material was left to stand overnight, was wet with petrol (500 ml) and diethyl ether (50 ml) and was stirred at room temperature for 1 hour. The solid was collected by filtration and washed with further petrol (200 ml). 21.2 g to give the product, Key Intermediate 3a as a white granular solid.

### Alternative Synthesis of Key Intermediate 3a

### 2-Methyl-propane-2-(R)-sulfinic acid [(R)-1-(4-chloro-2-fluoro-3-hydroxy-phenyl)-propyl]-amide

### Step 1

To a 5 L flange flask fitted with a stirrer bar and nitrogen inletoutlet was charged 2-chloro-6-fluorophenol (40 g, 273 mmol, 1.0 eq), DCM (1.1 L) and imidazole (28 g, 411 mmol, 1.5 eq). Tert-Butyldimethylsilyl chloride (41.13 g, 273 mmol, 1.0 eq) was added portionwise over 30 min at T<25° C. After 1 hour, TLC showed 5% 2-chloro-6-fluorophenol remained. Additional tert-butyldimethylsilyl chloride (2.0 g, 13.3 mmol, 0.05 eq) was added. After an additional stir of 1 hour, water (500 ml) was added and the organic layer separated. The organic layer was washed with 10% aq. citric acid (500 ml), 10% aq. K₂CO₃ (500 ml), then dried over MgSO₄, filtered and concentrated in vacuo to give a yellow oil (68 g). The material was purified by column chromatography on silica (500 g), eluting with heptanes (100%). The product fractions were concentrated and THF (200 ml) used to remove residual heptanes to give (2-chloro-6-fluorophenoxy)(tert-butyl)dimethylsilane as a colourless oil (64 g, 1H NMR >95%, 245 mmol, 90% yield). ¹H NMR (270 MHz, CDCl₃): 7.10 (1H, m), 6.90 (1H, m), 6.81 (1H, m), 1.04 (9H, s), 0.23 (6H, obs d).

### Step 2

To a 10 L flange flask fitted with a overhead anchor stirrer, temperature probe, dropping funnel and nitrogen inletoutlet was added (2-chloro-6-fluorophenoxy)(tert-butyl)dimethylsilane (176.4 g, 678 mmol, 1.0 eq) and THF (3.5 L). The solution was cooled to −70° C. and sec-butyllithium (1.4 M in cyclohexanes, 630 ml, 882 mmol, 1.3 eq) was added dropwise at <−65° C. After 2 hours, ¹H NMR indicated 13% starting material remained. An additional charge of sec-butyllithium (1.4 M in cyclohexanes, 82 ml, 115 mmol, 0.17 eq) was added. After 30 min, DMF (68 ml, 880 mmol, 1.3 eq) was added dropwise at <−65° C. After 30 min, the reaction was quenched by addition of acetic acid (180 ml) in THF (90 ml). The reaction was allowed to warm to −35° C. and water (1.4 L) was charged. The organic layer was separated off and the aqueous layer extracted with diethyl ether (1.4 L). The combined organic layers were washed with sat. brine (1.4 L) before being dried over MgSO₄, filtered and concentrated in vacuo. The material was purified by column chromatography on silica (2500 g), eluting with heptanes (100%) up to 100% EtOAc. The product fractions were concentrated to give 3-tert-butyldimethylsilanyloxy-4-chloro-2-fluorobenzaldehyde (157.3 g, ¹H NMR >95% excluding solvent, 86% active, 478 mmol, 69% yield). ¹H NMR (270 MHz, CDCl₃): 10.27 (1H, s), 7.40 (1H, dd), 7.23 (1H, dd), 1.04 (9H, s), 0.26 (6H, d).

### Step 3

To a 10 L flange flask fitted with overhead stirrer was added 3-tert-butyldimethylsilanyloxy-4-chloro-2-fluorobenzaldehyde (135.3 g 469 mmol, 1.0 eq) followed by DCM (2 L), (R)-(+)-2-methyl-2-propane sulfinamide (68.14 g, 562 mmol, 1.2 eq) and finally titanium (IV) ethoxide (213.7 g, 937 mmol, 2.0 eq). The straw coloured mixture was stirred at room temperature overnight under nitrogen. After overnight stirring the NMR confirmed the reaction to be complete. To the mixture was added DCM (2 L) followed by sodium sulphate decahydrate (1.36 Kg). The mixture was stirred vigorously for 1 hour before filtration through Celite (580 g). The Celite pad was washed well with DCM (3×2 L), the filtrate was evaporated to dryness at 40° C. and any residual water azeotroped with toluene (3×600 ml) to give 1-[3-(tert-butyl-dimethyl-silanyloxy)-4-chloro-2-fluoro-phenyl]-meth-(E)-ylideneamide as a yellow oil (192.5 g, ¹H NMR >95% excluding solvent, 91% active, 447 mmol, 95% yield). ¹H NMR (270 MHz, CDCl₃): 8.81 (1H, s), 7.50 (1H, dd), 7.30-7.15 (1H, m), 1.25 (9H, s), 1.04 (9H, s), 0.24 (6H, d).

### Step 4

To a 10 L flange flask fitted with stirrer bar, nitrogen inletoutlet and temperature probe was added (R)-2-methyl-propane-2-sulfinic acid 1-[3-(tert-butyl-dimethyl-silanyloxy)-4-chloro-2-fluoro-phenyl]-meth-(E)-ylideneamide (176 g, 448 mmol, 1.0 eq) followed by THF (3.2 L). The mixture was cooled to −78° C. before cannula addition of ethylmagnesium bromide (3.0 M in Et₂O, 269 ml, 895 mmol, 2.0 eq). During addition the mixture became opaque and thickened. The mixture was stirred for 3 hours after which time NMR confirmed the reaction to be complete. The mixture was quenched by the addition of sat. ammonium chloride (1.7 L). The mixture was extracted with EtOAc acetate (2×3 L), dried (MgSO₄), filtered and evaporated to dryness to give the crude product as a straw coloured oil (190 g). The crude product was adsorbed onto silica (400 g) and loaded onto a silica column (3000 g). The product was eluted using heptaneEtOAc, 0-20%. (R)-2-Methyl-propane-2-sulfinic acid {(R)-1-[3-(tert-butyl-dimethyl-silanyloxy)-4-chloro-2-fluoro-phenyl]-propyl}-amide, major diastereomer, was isolated as an off-white solid (98.8 g, ¹H NMR >95%, 234 mmol, 52% yield). ¹H NMR (270 MHz, CDCl₃): 7.10 (1H, dd), 6.81 (1H, dd), 4.45 (1H, q), 3.46 (1H, d), 2.05-1.65 (2H, m), 1.20 (9H, s), 1.03 (9H, s), 0.84 (3H, t), 0.21 (6H, d).

### Step 5

To a 5 L flange flask was charged (R)-2-methyl-propane-2-sulfinic acid {(R)-1-[3-(tert-butyl-dimethyl-silanyloxy)-4-chloro-2-fluoro-phenyl]-propyl}-amide (98.8 g, 234 mmol, 1.0 eq), water (1 L), MeCN (1 L) and cesium fluoride (52 g, 342 mmol, 1.46 eq). The mixture was stirred at room temperature overnight. The reaction was shown to be complete by HPLC. The MeCN was removed in vacuo and the residue acidified to pH 4 with citric acid (20 g). The aqueous was extracted with EtOAc (2×1 L). The organic layer was washed with sat. brine (1 L), dried over MgSO₄, filtered and concentrated in vacuo. Residual solvents were removed by a heptanes strip (500 ml) before the material was slurried in heptanes (600 ml) and filtered. The solid was washed with heptanes (100 ml) and dried in vacuo at 40° C. to give 66 g solid. This was reslurried in 4:1 heptaneEt₂O (640 ml) for 1 hour and filtered. The solids were washed with heptanes(2×100 ml) and dried to give to give (R)-2-methyl-propane-2-sulfinic acid [(R)-1-(4-chloro-2-fluoro-3-hydroxy-phenyl)-propyl]-amide (62.5 g, ¹H NMR >95%, 203 mmol, 87% yield). 1H NMR (270 MHz, CDCl₃): 8.03 (1H, bs), 7.00 (1H, dd), 6.65 (1H, dd), 4.45 (1H, q), 3.68 (1H, d), 1.95-1.65 (2H, m), 1.25 (9H, s), 0.83 (3H, t).

### Synthesis of Key Intermediate 3b

### 2-Methyl-propane-2-sulfinic acid [(R)-(4-chloro-2-fluoro-3-hydroxy-phenyl)-cyclopropyl-methyl]-amide

### Step 1

To 3-tert-butyldimethylsilanyloxy-4-chloro-2-fluorobenzaldehyde (12.49 g, 43.67 mmol, 1.0 eq) in DCM (200 ml) was added (S)-(−)-2-methyl-2-propane sulfinamide (5.30 g, 43.73 mmol, 1.0 eq) followed by titanium (IV) ethoxide (20.0 g, 87.68 mmol, 2.0 eq). The reaction was stirred overnight before addition of DCM (1000 ml) and sodium sulfate decahydrate (130 g). After 30 min vigorous stirring, the mixture was filtered through Celite (200 g) and the cake washed with DCM (2×500 ml). The organic liquors were dried (MgSO₄), filtered and concentrated. THF (300 ml) was charged to the crude compound and removed in vacuo to give (S)-2-methyl-propane-2-sulfinic acid 1-[3-(tertbutyl-dimethyl-silanyloxy)-4-chloro-2-fluoro-phenyl]-meth-(E)-ylideneamide (18.16 g, ¹H NMR >95% excluding solvent, 15.8 g active, 40.31 mmol, 92% yield). ¹H NMR (270 MHz, CDCl₃): 8.80 (1H, s), 7.49 (1H, dd), 7.18 (1H, dd), 1.25 (9H, s), 1.03 (9H, s), 0.24 (6H, d).

### Step 2

To a solution of (S)-2-methyl-propane-2-sulfinic acid 1-[3-(tertbutyl-dimethyl-silanyloxy)-4-chloro-2-fluoro-phenyl]-meth-(E)-ylideneamide (15.8 g, 40.31 mmol, 1.0 eq) in anhydrous THF (270 ml) at −75° C. was added 0.5M cyclopropylmagensium bromide in THF (161 ml, 80.5 mmol, 2.0 eq) dropwise at <−65° C. over 30 min. The reaction was stirred for 1 hour at <−65° C. before addition of saturated ammonium chloride solution (200 ml). The mixture was allowed to warm to 0° C. before extraction with EtOAc (3×200 ml). The combined organic layers were washed with sat. brine (200 ml), dried (MgSO₄), filtered and concentrated to give 20 g crude material (83:17 major:minor diastereomers by ¹H NMR). The crude material was adsorbed onto silica (30 g) and purified by column chromatography on silica (500 g), eluting with 10% EtOAcheptanesup to 80% EtOAc. (S)-2-Methyl-propane-2-sulfinic acid {(R)-[3-(tert-butyl-dimethyl-silanyloxy)-4-chloro-2-fluoro-phenyl]-cyclopropyl-methyl}-amide was isolated in two batches: 1^{st} batch; 7.77 g ¹H NMR >95% excluding solvent, 7.5 g active, 17.3 mmol, 43% yield. 2^{nd} batch; 3.04 g ¹H NMR >95% excluding solvent including 2% minor diastereomer, 2.87 g active, 6.61 mmol, 16% yield. 1H NMR (270 MHz, CDCl₃): 7.09 (1H, dd), 6.88 (1H, dd), 3.83 (1H, dd), 3.53 (1H, d), 1.27-1.20 (1H, m), 1.18 (9H, s), 1.03 (9H, s), 0.74-0.63 (1H, m), 0.57-0.37 (3H, m), 0.21 (6H, d).

### Step 3

To (S)-2-methyl-propane-2-sulfinic acid {(R)-[3-(tert-butyl-dimethyl-silanyloxy)-4-chloro-2-fluoro-phenyl]-cyclopropyl-methyl}-amide (7.50 g, 17.3 mmol, 1.0 eq) in MeCN (75 ml) was charged water (75 ml) and then cesium fluoride (3.15 g, 20.7 mmol, 1.2 eq) and the mixture stirred overnight at RT. The MeCN was removed in vacuo and 10% citric acid (30 ml) added (pH 4). The aqueous was extracted with EtOAc (2×40 ml) and the combined organic layers washed with sat. brine (20 ml) before being dried (MgSO₄), filtered and concentrated in vacuo. Heptane (50 ml) was charged and removed in vacuo. The crude solid was slurried in 1:1 heptanes:Et₂O (100 ml) for 1 hour at 0° C. before being filtered and washed with heptanes (20 ml). Oven drying at 40° C. gave (S)-2-methyl-propane-2-sulfinic acid [(R)-(4-chloro-2-fluoro-3-hydroxy-phenyl)-cyclopropyl-methyl]-amide (3.84 g, >97% by NMRLC, 12.0 mmol, 69% yield).

### Synthesis of Key Intermediate 3c

### [(R)-(4-Chloro-2-fluoro-phenyl)-cyclopropyl-methyl]-carbamic acid tert-butyl ester

### Step 1

To 4-chloro-2-fluorobenzaldehyde (30.64 g, 193.2 mmol, 1.0 eq) in DCM (460 ml) was added (S)-(−)-2-methyl-2-propane sulfinamide (23.41 g, 193.2 mmol, 1.0 eq) followed by titanium (IV) ethoxide (88.1 g, 386 mmol, 2.0 eq). The reaction was stirred overnight before addition of DCM (1 L) and sodium sulfate decahydrate (310 g). After 30 min vigorous stirring, the mixture was filtered through Celite (500 g) and the cake washed with DCM (2×1 L). The organic liquors were dried (MgSO₄), filtered and concentrated in vacuo. The crude compound was dissolved in DCM (500 ml), washed with 10% aq citric acid (200 ml), and saturated brine (100 ml), dried (MgSO₄), filtered and concentrated in vacuo to give (S)-2-methyl-propane-2-sulfinic acid 1-(4-chloro-2-fluoro-phenyl)-meth-(E)-ylideneamide (49.7 g, 1H NMR >95% excluding solvent, 46.7 g active, 178 mmol, 92% yield). 1H NMR (270 MHz, CDCl₃): 8.82 (1H, s), 7.96-7.90 (1H, dd), 7.24-7.16 (2H, m), 1.25 (9H, s).

### Step 2

To a solution of (S)-2-methyl-propane-2-sulfinic acid 1-(4-chloro-2-fluoro-phenyl)-meth-(E)-ylideneamide (26.2 g, 0.1 mol, 1.0 eq) in anhydrous THF (700 ml) at −75° C. was added 0.5M cyclopropylmagensium bromide in THF (400 ml, 0.2 mol, 2.0 eq) dropwise at <−65° C. over 30 min. The reaction was stirred for 2 hours at <−65° C. then allowed to warm to room temperature and stirred for 4 hours. Saturated ammonium chloride solution (300 ml), was added, followed by water (150 ml). The layers were separated and the aqueous extracted with EtOAc (3×200 ml). The combined organic layers were washed with sat. brine (300 ml), dried (MgSO₄), filtered and concentrated in vacuo. The crude material was purified by column chromatography on silica (500 g), eluting with 10% EtOAcheptanesup to 80% EtOAc. (S)-2-Methyl-propane-2-sulfinic acid [(R)-(4-chloro-2-fluoro-phenyl)-cyclopropyl-methyl]-amide was isolated in two batches (combined yield 26.4 g, 86.9 mmol, 87%): 1^{st} batch; 18.4 g ¹H NMR 4:1 mixture of diastereomers in favour of desired isomer, 2^{nd} batch; 8 g ¹H NMR 19:1 mixture of diastereomers in favour of desired isomer. The 2^{nd} batch was repurified by column chromatography on silica (500 g), eluting with 10% EtOAcheptanesup to 80% EtOAc, to give 6.6 g of pure (S)-2-methyl-propane-2-sulfinic acid [(R)-(4-chloro-2-fluoro-phenyl)-cyclopropyl-methyl]-amide. 1H NMR (270 MHz, CDCl₃): 7.33 (1H, t), 7.11 (1H, dd), 7.08 (1H, dd), 3.86 (1H, dd), 3.56 (1H, d), 1.28-1.22 (1H, m), 1.18 (9H, s), 0.90-0.80 (1H, m), 0.74-0.64 (1H, m), 0.56-0.35 (2H, m).

### Step 3

To a solution of (S)-(−)-2-methyl-propane-2-sulfinic acid [(R)-(4-chloro-2-fluoro-phenyl)-cyclopropyl-methyl]-amide (6.6 g, 21.7 mmol, 1.0 eq) in EtOAc (150 ml) was added 2.1 M HCl in EtOAc (20.7 ml, 43.4 mmol, 2.0 eq) and the mixture stirred overnight, after which time analysis indicated complete deprotection. The mixture was concentrated in vacuo, the residue slurried in heptaneEt₂O (31, 100 ml) for 1 hour, filtered and sucked dry. The HCl salt was partitioned between DCM (100 ml) and sat aq NaHCO₃ (50 ml) and the mixture stirred vigorously for 10 min, the layers separated and the aqueous extracted with DCM. The combined organics were dried (MgSO₄), filtered and concentrated in vacuo. The resulting amine (3.6 g, 18.0 mmol, 1.0 eq) was dissolved in THF (60 ml) and Et₃N (3.8 ml, 27.0 mmol, 1.5 eq) added, followed by Boc₂O (5.17 g, 23.4 mmol, 1.3 eq). The mixture was stirred at room temperature for 1 hour, additional Boc₂O (0.5 g) added and the mixture stirred for an additional 1 hour, after which time analysis (LC) indicated complete conversion. Water (60 ml) was added, the layers separated and the aqueous extracted with EtOAc (2×60 ml). The combined organics were dried (MgSO₄), filtered and concentrated. The residue was purified on silica (150 g) eluting with 100% heptanes to 20% EtOAcheptane. The isolated material was slurried in heptanes (30 ml), the solid filtered, washed with heptanes and sucked dry to give [(R)-(4-chloro-2-fluoro-phenyl)-cyclopropyl-methyl]-carbamic acid tert-butyl ester (1.9 g). The filtrate was concentrated and the solid obtained reslurried in heptanes (10 ml) to provide additional [(R)-(4-chloro-2-fluoro-phenyl)-cyclopropyl-methyl]-carbamic acid tert-butyl ester (1.2 g, combined yield 3.1 g, 10.3 mmol, 47.7%).

### Synthesis of Key Intermediate 3d

### [(R)-1-(4-Chloro-2-fluoro-phenyl)-propyl]-carbamic acid tert-butyl ester

### Step 1

To a solution of 4-chloro-2-fluoro-benzaldehyde (198.9 g, 1.254 mol, 1.0 eq) in DCM (2.5 ml) was added (R)-(+)-2-methyl-2-propanesulfinamide (159.6 g, 1.317 mol, 1.1 eq). To this was added a solution of titanium (IV) ethoxide (571.8 g, 2.008 mol, 1.6 eq) in DCM (500 ml) and the reaction was stirred at room temperature overnight. The reaction was diluted with DCM (2 L), Na₂SO₄.10H₂O (2.00 Kg, 6.21 mol, 5.0 eq) was added and the mixture was stirred for 1 h. The mixture was filtered through Celite (1 Kg), eluting with DCM (2×2 L). The filtrate was concentrated in vacuo and the sample dissolved in DCM (2 L). The solution was washed with 10% citric acid solution (2×500 ml) and water (500 ml), dried over MgSO₄, filtered and concentrated in vacuo. The residue was slurried in heptanes (200 ml) at 40° C. for 1 hour and then cooled to room temperature and stirred overnight. The stirred suspension was cooled to 0° C. for 1 hour then filtered, washed with cold heptanes (50 ml) and dried in an oven at 40° C. under vacuum overnight to give 237 g of material. The filtrate was concentrated in vacuo, the residue recrystallised from refluxing heptanes (100 ml), cooled to 0° C., filtered and washed with cold heptanes (20 ml). The solids were dried in an oven at 40° C. under vacuum overnight to give 14.1 g of material which was blended with the 237 g previously isolated to give (R)-(+)-2-methyl-propane-2-sulfinic acid 1-(4-chloro-2-fluoro-phenyl)-meth-(E)-ylideneamide (256.7 g, ¹H NMR >95%, 0.981 mol, 78% yield). ¹H NMR (270 MHz, CDCl₃): 8.82 (1H, s), 7.96-7.90 (1H, m), 7.25-7.17 (2H, m), 1.25 (9H, s).

### Step 2

To a solution of (R)-(+)-2-methyl-propane-2-sulfinic acid 1-(4-chloro-2-fluoro-phenyl)-meth-(E)-ylideneamide (50 g, 0.191 mol, 1.0 eq) in THF (1 L) at −75° C. was added 3M ethylmagnesium bromide in Et₂O (127.4 ml, 0.382 mol, 2.0 eq) slowly at <−65° C. over 30 min. The reaction was stirred for 2.5 h at <−65° C. before addition of sat. ammonium chloride solution (500 ml). The solution was diluted with water (250 ml) and the organic layer separated. The aqueous layer was extracted with EtOAc (2×500 ml) and the combined organic layers were washed with brine (500 ml), dried over MgSO₄, filtered and concentrated in vacuo to afford 59 g of crude material (3:1 mixture of diastereomers by ¹H NMR). The crude material was purified by chromatography (silica, 1 Kg) eluting with 20% EtOAcheptanes up to 30% EtOAc to give (R)-(+)-2-methyl-propane-2-sulfinic acid [(R)-1-(4-chloro-2-fluoro-phenyl)-propyl]-amide (19.9 g, ¹H NMR >95%, 0.0682 mol, 34% yield). ¹H NMR (270 MHz, CDCl₃): 7.27-7.21 (1H, m), 7.13-7.04 (2H, m), 4.43 (1H, q), 3.50 (1H, d), 2.02-1.72 (2H, m), 1.21 (9H, s), 0.89 (3H, t).

### Step 3

To a solution of (R)-(+)-2-methyl-propane-2-sulfinic acid [(R)-1-(4-chloro-2-fluoro-phenyl)-propyl]-amide (19.9 g, 68.2 mmol, 1.0 eq) in EtOAc (500 ml) was added 2.1M HCl in dioxane (69 ml, 137.1 mmol, 2.0 eq) slowly. The reaction was stirred at room temperature under N₂ for 30 min. The solvents were removed in vacuo and the crude material slurried in 3:1 heptane:Et₂O (200 ml) for 20 min then filtered and the cake washed with heptanes (2×50 ml). The cake was dried in an oven at 35° C. under vacuum for 30 min to give (R)-1-(4-chloro-2-fluoro-phenyl)-propylamine hydrochloride (19.6 g, ¹H NMR >95% excluding solvents, 77% active, 67.7 mmol, 99% yield). ¹H NMR (270 MHz, DMSO-d₆): 8.81 (3H, s), 7.77 (1H, t), 7.52 (1H, dd), 7.41 (1H, dd), 4.33 (1H, q), 2.08-1.76 (2H, m), 0.76 (3H, t).

### Step 4

To a suspension of (R)-1-(4-chloro-2-fluoro-phenyl)-propylamine hydrochloride (19.6 g, 67.7 mmol, 1.0 eq) in THF (330 ml) at room temperature was added di-tert-butyl dicarbonate (19.8 g, 90.7 mmol, 1.3 eq) and the reaction was stirred at room temperature overnight. To this was added water (330 ml) and EtOAc (330 ml). The layers were separated, the aqueous layer was extracted with EtOAc (330 ml), the combined organics were washed with brine (330 ml), dried over MgSO₄, filtered, and concentrated in vacuo. The residue was dissolved in EtOAc (100 ml) and washed with an aqueous 10% citric acid solution (2×50 ml), dried over MgSO₄, filtered and concentrated in vacuo. The residue was triturated with 5:1 heptaneEtOAc (100 ml) to give a white crystalline solid which was slurried in heptanes (100 ml) to give 5 g of material. The liquors were concentrated in vacuo then slurried in heptanes (50 ml) to give 10 g of material. The liquors were concentrated in vacuo and then slurried in heptanes (10 ml) to give 3.9 g of material. The collected solids were oven dried at 45° C. under vacuum for 6 h to give 15.8 g of material. Of this, 9.2 g was dissolved in DCM (200 ml), washed with water (3×100 ml) and brine (100 ml), dried over MgSO₄, filtered and concentrated in vacuo to provide [(R)-1-(4-chloro-2-fluoro-phenyl)-propyl]-carbamic acid tert-butyl ester (8.7 g, ¹H NMR >95%, 30.2 mmol 77% yield).

### Synthesis of Key Intermediate 3e

### [(R)-1-(4-Chloro-2-fluoro-3-hydroxy-phenyl)-propyl]-carbamic acid tert-butyl ester

### Step 1

To a solution of (R)-2-methyl-propane-2-sulfinic acid [(R)-1-(4-chloro-2-fluoro-3-hydroxy-phenyl)-propyl]-amide (20 g, 64.9 mmol, 1 eq) in EtOAc (500 ml) and MeOH (40 ml) was added 2.1M HCl in EtOAc (62 ml, 130 mmol, 2 eq) slowly. The reaction was stirred for 1 h at RT then concentrated in vacuo. To the oil was added 3:1 heptane:Et₂O (500 ml) and the solution stirred for 5 min at 40° C. then concentrated in vacuo. To the solid residue was added 3:1 heptane:Et₂O (400 ml) and the solution stirred for 5 min at 40° C. then concentrated in vacuo. The solid was slurried in 3:1 heptane:Et₂O (200 ml) at RT overnight, filtered and the solids washed with heptanes (3×50 ml) to give 15.7 g of material. This was dissolved in THF (330 mL) and to the stirred solution was added Et₃N (20 ml, 66.48 mmol, 1.02 eq). To the mixture was added di-tert-butyl dicarbonate (15.6 g, 71.48 mmol, 1.1 eq) and the reaction was stirred for 1 h at RT. Di-tert-butyl dicarbonate (0.78 g, 3.57 mmol, 0.06 eq) and Et₃N (1 ml, 3.32 mmol, 0.05 eq) were added and the reaction stirred for 1 h. Upon completion, H₂O (330 ml) was added and the organics were extracted with EtOAc (2×330 ml), washed with brine (330 ml), dried over MgSO₄, filtered and concentrated in vacuo. The residue was purified via chromatography (silica, 380 g) and the concentrated product fractions were azeotroped with heptanes (2×300 ml) to give an oily solid. The material was dissolved in 20% THF80% MeOH (350 ml) and 2M KOH (350 ml) solution was added and the reaction was stirred at RT overnight. 10% Aq. citric acid (515 ml) was added (pH 4) and the organics were extracted with EtOAc (2×1 L), washed with brine, dried over MgSO₄, filtered and concentrated in vacuo to give [(R)-1-(4-chloro-2-fluoro-3-hydroxy-phenyl)-propyl]-carbamic acid tert-butyl ester as an off white solid (20 g, ¹H NMR ca. 95%, 62.6 mmol, 96% yield).

### Synthesis of Key Intermediate 3fq

### [(R)-1-(4-Chloro-2-fluoro-3-iodo-phenyl)-propyl]-carbamic acid tert-butyl ester and 3((R)-1-tert-Butoxycarbonylamino-propyl)-6-chloro-2-fluoro-benzoic acid butyl ester

### Step 1—Intermediate 3f

To a flame dried flask under N₂ was charged a solution of [(R)-1-(4-chloro-2-fluoro-phenyl)-propyl]-carbamic acid tert-butyl ester (1.40 g, 4.84 mmol, 1.0 eq) in THF (36 ml). The stirred solution was cooled to −78° C. To this was added 2.5M n-butyllithium in hexane (4.25 ml, 10.64 mmol, 2.2 eq) dropwise <−65° C. over 5 min. The reaction was allowed to warm to −59° C. then cooled to <−65° C. for 1.5 h. To this was added a solution of I₂ (1.35 g, 5.32 mmol, 1.1 eq) in THF (6 ml) over 30 seconds. The reaction was stirred at <−65° C. for 30 min then quenched with water (45 ml) and allowed to warm to room temperature. The mixture was diluted with sat. aq. sodium thiosulphate (40 ml) then extracted with EtOAc (2×100 ml). The combined organics were washed with brine (100 ml), dried over MgSO₄, filtered and concentrated in vacuo. The residue was purified by chromatography (silica, 220 g) eluting with 1% MeOH7% EtOAc92% heptanes to give [(R)-1-(4-Chloro-2-fluoro-3-iodo-phenyl)-propyl]-carbamic acid tert-butyl ester (1.02 g, ¹H NMR >95%, 2.34 mmol, 48% yield).

### Step 2—Intermediate 3 g

To a solution of [(R)-1-(4-chloro-2-fluoro-3-iodo-phenyl)-propyl]-carbamic acid tert-butyl ester (200 mg, 0.484 mmol, 1 eq) in ⁿBuOH (10 ml) was added PdCl₂ (5 mg, 0.027 mmol, 5 mol %), 1,3-bis(diphenylphosphino)propane (11 mg, 0.027 mmol, 5 mol %) and 1,8-diazabicyclo[5.4.0]undec-ene (0.08 ml, 0.535 mmol, 1.1 eq). The mixture was sparged with CO₂ and heated to 100° C. for 1.5 hours. The reaction was cooled to room temperature and sparged with N₂. The mixture was filtered through Celite and washed with MeOH (2×50 ml). The filtrate was concentrated in vacuo and the residue was passed through a pad of silica (10 g) eluting with 1:1 EtOAc:heptane. Product containing fractions were concentrated in vacuo to give 3-((R)-1-tert-butoxycarbonyl-mino-propyl)-6-chloro-2-fluoro-benzoic acid butyl ester (105 mg, ¹H NMR >95%, 0.257 mmol, 53% yield).

### Synthesis of Key Intermediate 3 h

### [(R)-1-(4-Chloro-2-fluoro-phenyl-3-boronic acid)-propyl]-carbamic acid tert-butyl ester

### Step 1

To a solution of [(R)-1-(4-chloro-2-fluoro-phenyl)-propyl]-carbamic acid tert-butyl ester (1.00 g, 3.48 mmol, 1.0 eq) in THF (30 ml) at −70° C. was added n-Butyllithium (2.5M in hexanes, 1.39 ml, 3.48 mmol, 1.0 eq) at <−65° C. over 5 mins. After stirring for 10 mins, sec-Butyllithium (1.4M in cyclohexane, 2.74 ml, 3.84 mmol, 1.1 eq) was added dropwise over 5 mins at <−65° C. After 1 hour, 2-Isopropoxy-4,4,5,5-tetramethyl-1,3,2-dioxaborolane (1.29 g, 6.95 mmol, 2.0 eq) was added as a solution in THF (2 ml) at <−65° C. The reaction was stirred for 3 hours then quenched by addition of sat. ammonium chloride solution (20 ml). The mixture was allowed to warm to 0° C., before addition of water (10 ml) and extraction with Et₂O (2×30 ml). The organic layer was washed with sat. brine (30 ml), dried (MgSO₄), filtered and concentrated in vacuo. The crude material was purified by column chromatography on silica (50 g), eluting with 100% DCM. The product fractions were concentrated to give {(R)-1-[4-chloro-2-fluoro-3-(4,4,5,5-tetramethyl-[1,3,2]dioxaborolan-2-yl)-phenyl]-propyl}-carbamic acid tert-butyl ester (490 mg, 1H NMR >95% excluding solvents, 88% active, 1.04 mmol, 30% yield). 1H NMR (270 MHz, CDCl₃): 7.20-7.02 (2H, m), 4.90 (1H, bs), 4.65 (1H, bs), 1.80-1.65 (2H, m), 1.45-1.30 (21H, m), 0.84 (3H, t).

### Step 2

To {(R)-1-[4-chloro-2-fluoro-3-(4,4,5,5-tetramethyl-[1,3,2]dioxaborolan-2-yl)-phenyl]-propyl}-carbamic acid tert-butyl ester (340 mg, 0.821 mmol, 1.0 eq) in acetone (30 ml) and water (30 ml) was added ammonium acetate (127 mg, 1.642 mmol, 2.0 eq) and then sodium metaperiodate (351 mg, 1.642 mmol, 2.0 eq). After stirring for 2 hour at 20° C., the acetone was removed in vacuo. The pH was adjusted to ˜5 with 10% citric acid solution (5 ml) and extracted with DCM (20 ml and 10 ml). The organic layer was washed with sat. brine (5 ml), dried (MgSO₄), filtered and concentrated to give a crude material (381 mg). The crude material was combined with a previous batch (350 mg crude) and was purified by column chromatography on silica (9 g) eluting 100% DCM up to 2% MeOHDCM. The product containing fractions were concentrated to give [(R)-1-(4-chloro-2-fluoro-phenyl-3-boronic acid)-propyl]-carbamic acid tert-butyl ester (330 mg).

### Synthesis of Key Intermediate 4

### (2,4-Difluoro-3-hydroxy-benzyl)-carbamic acid tert-butyl ester

### Step 1

48% HBr (10 ml) was added to 2,4 difluoro-3-methoxybenzylamine (1 g, 5.78 mmol) and heated to 145° C. for 1 hour, mixture concentrated and triturated with ethyl acetate to afford 3-aminomethyl-2,6-difluoro-phenol (1.2 g)) MS: [M+H]⁺160

### Step 2

A solution of di-tert-butyldicarbonate (10.91 g, 0.05 mol) in tetrahydrofuran (60 ml) was added dropwise over 1 h to an ice cold mixture of 3-aminomethyl-2,6-difluoro-phenol (12 g, 0.05 mol), tetrahydrofuran (60 ml), water (120 ml) and 6M sodium hydroxide (21 ml, 0.125 mol). The mixture was warmed to RT, acidified with 5% citric acid (240 ml) and extracted with ethyl acetate (2×120 ml). The combined organic phase was washed with sat. brine (120 ml), dried over magnesium sulphate, filtered and concentrated. The residue was triturated with petrol, filtered and dried to give Key Intermediate 4 (13.9 g).

### Synthesis of Key Intermediate 5

### 4-(3-Aminomethyl-2,6-difluoro-phenoxy)-phenylamine

### Step 1

To a suspension of (2,4-difluoro-3-hydroxy-benzyl)-carbamic acid tert-butyl ester (Key Intermediate 4) (200 mg, 0.77 mmol), 4-fluoronitrobenzene (88 mg, 0.77 mmol) and potassium carbonate (213 mg, 1.15 mmol) in DMSO (4 ml) was stirred at 115° C. overnight. The mixture was partitioned between water and ethyl acetate, organic fraction dried over sodium sulphate, filtered and concentrated. Residue purified by column chromatography to give [2,4-difluoro-3-(4-nitro-phenoxy)-benzyl]-carbamic acid tert-butyl ester MS: [M+H]⁺381.

### Step 2

[2,4-Difluoro-3-(4-nitro-phenoxy)-benzyl]-carbamic acid tert-butyl ester was reduced as described in Example 19 step 2 to give [3-(4-amino-phenoxy)-2,4-difluoro-benzyl]-carbamic acid tert-butyl ester. MS: [M+Na]⁺373.

### Step 3

[3-(4-Amino-phenoxy)-2,4-difluoro-benzyl]carbamic acid tert-butyl ester was hydrolysed as described in Example 19 step 3 to give 4-(3-aminomethyl-2,6-difluoro-phenoxy)-phenylamine.

### Synthesis of Key Intermediate 6

### 3-(Benzo[1,3]-dioxol-5-yloxy)-2,4-difluoro-benzylamine

### Step 1

(2,4-Difluoro-3-hydroxy-benzyl)-carbamic acid tert-butyl ester (Key Intermediate 4) (0.1 g, 0.386 mmol) was treated with 2,3-dihydro-1-benzofuran-5-ylboronic acid (0.126 g, 0.771 mmol) using the method described in Key Intermediate 1, step 1 to give [3-(benzo[1,3]-dioxol-5-yloxy)-2,4-difluoro-benzyl]-carbamic acid tert-butyl ester, 33 mg. MS: [M+Na]⁺401.

### Step 2

[3-(Benzo[1,3]dioxol-5-yloxy)-2,4-difluoro-benzyl]-carbamic acid tert-butyl ester (0.067 g, 0.178 mmol) was treated with HCl as described in Example 3, step 3 to yield 3-(benzo[1,3]dioxol-5-yloxy)-2,4-difluoro-benzylamine, 28 mg.

### Synthesis of Key Intermediate 7

### 4-Fluoro-3-phenoxy-benzylamine

A solution of 4-fluoro-3-methoxybenzylamine hydrochloride (925 mg) in 48% aqueous hydrogen bromide was heated at reflux for 4 hours then evaporated to dryness to give 1.05 g of 5-aminomethyl-2-fluorophenol hydrobromide. A solution of 5-aminomethyl-2-fluorophenol hydrobromide (1.05 g; 4.75 mmol), phthaloyl dichloride (720 μl; 5 mmol) and triethylamine (2.4 ml; 17 mmol) in toluene was heated at 100° C. for 48 hours. The reaction mixture was cooled then partitioned between EtOAc and 2M hydrochloric acid. The EtOAc layer was separated, washed with saturated NaHCO₃ solution, then dried over Na₂SO₄, filtered and evaporated. The crude material was purified by flash column chromatography, gradient elution from 0% to 60% EtOAc in petroleum ether. Product containing fractions were combined and evaporated to give 540 mg of 2-(4-fluoro-3-hydroxy-benzyl)-isoindole-1,3-dione. [MH]⁺=272. 2-(4-Fluoro-3-phenoxy-benzyl)-isoindole-1,3-dione was prepared in a manner analogous to that of key intermediate 1, step 1, but starting from 2-(4-fluoro-3-hydroxy-benzyl)-isoindole-1,3-dione. [MH]⁺=348.

A solution of 2-(4-fluoro-3-phenoxy-benzyl)isoindole-1,3-dione (110 mg) and hydrazine hydrate (20 μl) in ethanol (5 ml) was heated at 60° C. overnight. The reaction mixture was evaporated then purified by preparative LCMS top give Key Intermediate 7. [MH]⁺=201.

### Synthesis of Key Intermediates 8 and 9

### (S)-3-(4-Chloro-2-fluoro-3-phenoxy-phenyl)-3-((R)-2-methyl-propane-2-sulfinylamino)-propionic acid and (R)-3-(4-Chloro-2-fluoro-3-phenoxy-phenyl)-3-(R)-2-methyl-propane-2-sulfinylamino)-propionic acid

Anhydrous methyl acetate (0.67 ml, 8.4 mmol) was added to a cooled solution of sodium hexadimethylsilazide (4.2 ml of a 2M solution in THF, 8.4 mmol) in diethyl ether (10 ml) at −78° C. under an inert atmosphere. The resulting solution was stirred 45 min further at this temperature and a solution of 2-methyl-propane-2-sulfinic acid 1-(4-chloro-2-fluoro-3-phenoxy-phenyl)-methylideneamide (1.5 g, 4.2 mmol) in diethyl ether (15 ml) was added. The reaction was stirred for 2 hours at −78° C., quenched with sat. ammonium chloride and allowed to warm to room temperature. The reaction mixture was concentrated under reduced pressure and the residue repartitioned between DCM and water. The layers were separated and the organic fraction evaporated to dryness. Trituration of the residue with ethyl acetate gave 1.05 g of a single diastereoisomer as a colourless powder. The relative stereochemistry was confirmed to be RₛS by small molecule X-ray crystallography.

The filtrate was evaporated and the residue dissolved in 1:1 THFMeOH (10 ml) and treated with 1M LiOH (8 ml) at room temperature overnight. The solvent was evaporated and the residue ripartitioned between Et₂O and H₂O, the aqueous layer was separated, acidified with 5% HCl (aq) and extracted with DCM. The combined organic extract was washed with H₂O, dried over Na₂SO₄, filtered and evaporated. Trituration of the crude residue with EtOAc gave 0.24 g of the second diastereoisomer as a colourless powder. The relative stereochemistry was confirmed to be R_{S}R by small molecule X-ray crystallography.

### Synthesis of Key Intermediate 10

### 4-Chloro-2-fluoro-3-phenoxy-benzoic acid

To a solution of 2,4-di fluoro-3-phenoxybenzaldehyde as described in key intermediate 1 (100 mg, 0.4 mmol) in acetic acid (1 ml) at 50° C. was added sodium perborate tetrahydrate (74 mg, 0.48 mmol) portionwise over 15 minutes, heating continued for 4 hours and left at 48 hours at RT. Precipitated solid filtered and washed with diethyl ether to give the product, Key Intermediate 10 (43 mg).

### Synthesis of Key Intermediate 11

### (Z)-3-[(R)-1-(4-Chloro-2-fluoro-3-phenoxy-phenyl)-propylamino]-but-2-enoic acid methyl ester

A solution of (R)-1-(4-Chloro-2-fluoro-3-phenoxy-phenyl)-propylamine (250 mg; 0.9 mmol), methyl acetoacetate (115 μl; 1.2 equivalents) and acetic acid (25 μl; 0.5 equivalents) in methanol (10 ml) was heated at 60° C. overnight then evaporated. The residue was partitioned between EtOAc and sat sodium hydrogen carbonate solution, the EtOAc layer was separated, then dried over Na₂SO₄, filtered and evaporated to give 330 mg of (Z)-3-[(R)-1-(4-Chloro-2-fluoro-3-phenoxy-phenyl)-propylamino]-but-2-enoic acid methyl ester as a colourless gum.

### Synthesis of Key Intermediates 12 and 13

### (R)-3-[(R)-1-(4-Chloro-2-fluoro-3-phenoxy-phenyl)-propylamino]-butyric acid methyl ester and

### (S)-3-[(R)-1-(4-Chloro-2-fluoro-3-phenoxy-phenyl)-propylamino]-butyric acid methyl ester

(R)-1-(4-Chloro-2-fluoro-3-phenoxy-phenyl)-propylamine hydrochloride (20 g, 63.2 mmol) (prepared in an analogous manner to KI-1) was converted to the free-base by partition between CHCl₃ and sat sodium hydrogen carbonate solution, the phases were separated and the aqueous layer was extracted into CHCl₃ (×2). Combined organic extracts were dried (magnesium sulfate), filtered and concentrated. The (R)-1-(4-chloro-2-fluoro-3-phenoxy-phenyl)-propylamine was split into two equal portions and methyl crotonate (60 ml) was added to each portion. Each reaction was heated to reflux, stirring under nitrogen for 24 h. The combined mixture was concentrated, azeoptroping with toluene. The residue was chromatographed twice, first eluting with a gradient of 10% EtOAcpetrol to 40% EtOAcpetrol to give a preliminary purification; the second with a gradient of toluene to 40% n-butyl actetatetoluene to give: (R)-3-[(R)-1-(4-chloro-2-fluoro-3-phenoxy-phenyl)-ethylamino]-butyric acid methyl ester (8.89 g). Upon further elution, (S)-3-[(R)-1-(4-Chloro-2-fluoro-3-phenoxy-phenyl)-ethylamino]-butyric acid methyl ester (7.69 g) was isolated.

### Table of Key Intermediates

By following the methods described above or in the Examples or General Methods below, or methods analogous thereto, Key Intermediates K-1 to K-30 were prepared.

| Inter- | | | | |
|---|---|---|---|---|
| me- | | | | |
| diate | Structure | Name | NMR & MS Data | Synthetic Method |
| KI-1 | 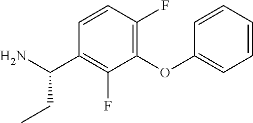 [SMILES: CC[C@H](N)c1ccc(F)c(Oc2ccccc2)c1F] | (S)-1-(2,4- Difluoro-3- phenoxy-phenyl)- propylamine | ¹H NMR (400 MHz, DMSO- d₆): 8.57 (2H, s), 7.66-7.56 (1H, m), 7.53-7.43 (1H, m), 7.43-7.34 (2H, m), 7.14 (1H, t), 6.97 (2H, d), 4.40 (1H, s), 2.07-1.96 (1H, m), 1.93-1.80 (1H, m), 0.82 (3H, t). | Key Intermediate 1 |
| KI-2 | 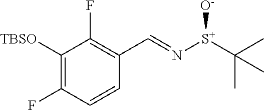 [SMILES: CC(C)(C)[S@+]([O-])/N=C/c1ccc(F)c(O[Si](C)(C)C(C)(C)C)c1F] | 2-Methyl- propane-2-(S)- sulfinic acid 1-[3-(tert-butyl- dimethyl- silanyloxy)- 2,4-difluoro- | ¹H NMR (400 MHz, DMSO- d₆): 8.62 (1H, s), 7.64 (1H, q), 7.36-7.24 (1H, m), 1.19 (9H, s), 1.00 (9H, s), 0.21 (6H, s). MS: [M + H]⁺ 376. | Key Intermediate 2 |
| | | phenyl]-meth- | | |
| | | (E)-ylideneamide | | |
| KI-3a | 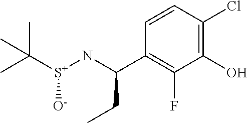 [SMILES: CC[C@@H](N[S@+]([O-])C(C)(C)C)c1ccc(Cl)c(O)c1F] | 2-Methyl- propane- 2-(R)-sulfinic acid [(R)-1-(4- chloro-2-fluoro- 3-hydroxy- phenyl)-propyl]- amide | 1H NMR (400 MHz, DMSO- d6): 10.31-10.17 (1H, m), 7.16 (1H, d), 6.93 (1H, t), 5.62 (1H, d), 4.32 (1H, q), 1.86-1.73 (1H, m), 1.70-1.57 (1H, m), 1.10 (9H, s), 0.84 (3H, t). | Key Intermediate 3a |
| KI-3b | 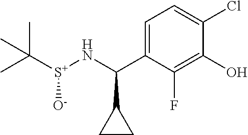 [SMILES: CC(C)(C)[S@@+]([O-])N[C@@H](c1ccc(Cl)c(O)c1F)C1CC1] | 2-Methyl- propane- 2-sulfinic acid [(R)-(4-chloro- 2-fluoro-3- hydroxy- phenyl)- cyclopropyl- methyl]-amide | 1H NMR (270 MHz, CDCl₃): 7.08 (1H, dd), 6.85 (1H, dd), 6.63 (1H, s), 3.90 (1H, dd), 3.61 (1H, d), 1.27-1.15 (10H, m), 0.75-0.65 (1H, m), 0.60- 0.35 (3H, m). LCMS: 99.2% (320.1 MH⁺) | Key Intermediate KI-3b |
| KI-3c | 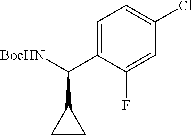 [SMILES: CC(C)(C)OC(=O)N[C@@H](c1ccc(Cl)cc1F)C1CC1] | [(R)-(4-Chloro-2- fluoro-phenyl)- cyclopropyl- methyl]-carbamic acid tert-butyl ester | 1H NMR (270 MHz, CDCl₃): 7.21 (1H, dd), 7.11-7.04 (2H, m), 5.11 (1H, br s), 4.23-4.12 (1H, m), 3.61 (1H, d), 1.38 (9H, br s), 1.20-1.11 (1H, m), 0.62-0.25 (4H, m). LCMS: 99.1% (322 MNa⁺) | Key Intermediate KI-3c |
| KI-3d | 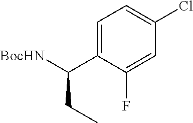 [SMILES: CC[C@@H](NC(=O)OC(C)(C)C)c1ccc(Cl)cc1F] | [(R)-1-(4-Chloro- 2-fluoro-phenyl)- propyl]- carbamic acid tert-butyl ester | 1H NMR (270 MHz, CDCl₃): 7.20-7.03 (3H, m), 4.93 (1H, s), 4.68 (1H, d), 1.77-1.69 (2H, m), 1.40 (9H, s), 0.88 (3H, t). MS: 310.0 ([M + Na]⁺). | Key Intermediate KI-3d |
| KI-3e | 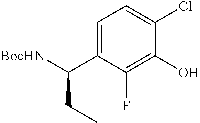 [SMILES: CC[C@@H](NC(=O)OC(C)(C)C)c1ccc(Cl)c(O)c1F] | [(R)-1-(4-Chloro- 2-fluoro-3- hydroxy-phenyl)- propyl]- carbamic acid tert-butyl ester | 1H NMR (270 MHz, CDCl₃): 7.06 (1H, dd), 6.72 (1H, t), 5.78 (1H, bs), 4.96-4.94 (1H, m), 4.81-4.73 (1H, m), 1.75- 1.67 (2H, m), 1.41 (9H, m), 0.86 (3H, t). MS: 326.1 ([M + Na]⁺). | Key Intermediate KI-3e |
| KI-3f | 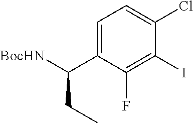 [SMILES: CC[C@@H](NC(=O)OC(C)(C)C)c1ccc(Cl)c(I)c1F] | [(R)-1-(4-Chloro- 2-fluoro-3-iodo- phenyl)-propyl]- carbamic acid tert-butyl ester | 1H NMR (270 MHz, CDCl₃): 7.25-7.14 (2H, m), 4.93 (1H, bs), 4.71-4.66 (1H, m), 1.80- 1.69 (2H, m), 1.40 (9H, s), 0.89 (3H, t). MS: 436.0 ([M + Na]⁺). | Key Intermediate KI-3f |
| KI-3g | 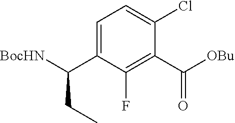 | 3-((R)-1-tert- Butoxycarbonyl- amino-propyl)- 6-chloro-2- fluoro-benzoic acid butyl ester | 1H NMR (270 MHz, CDCl₃): 7.27-7.14 (2H, m), 4.95-4.92 (1H, m), 4.71-4.69 (1H, m), 4.37 (2H, t), 1.79-1.63 (4H, m), 1.55-1.40 (11H, m), 0.98- 0.87 (6H, m). MS: 410.1 ([M + Na]⁺). | Key Intermediate KI-3g |
| KI-3h | 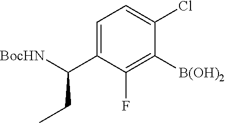 [SMILES: CC[C@@H](NC(=O)OC(C)(C)C)c1ccc(Cl)c(B(O)O)c1F] | [(R)-1-(4-Chloro- 2-fluoro-phenyl- 3-boronic acid)- propyl]-carbamic acid tert-butyl ester | 1H NMR (270 MHz, CDCl₃): 7.24-7.05 (2H, m), 4.95 (1H, bs), 4.66 (1H, bs), 3.64 (2H, s), 1.82-1.66 (2H, m), 1.39 (9H, bs), 0.87 (3H, t). LCMS: 354.1 (MNa⁺). | Key Intermediate KI-3h |
| KI-4 | 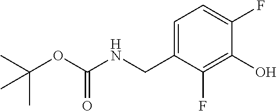 [SMILES: CC(C)(C)OC(=O)NCc1ccc(F)c(O)c1F] | (2,4-Difluoro-3- hydroxy-benzyl)- carbamic acid tert-butyl ester | 1H NMR (400 MHz, CDCl3): 6.93-6.76 (2H, m), 4.89 (1H, bs), 4.34 (2H, s), 1.47 (9H, s). | Key Intermediate 4 |
| KI-5 | 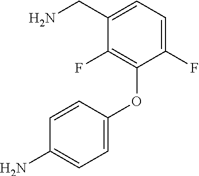 [SMILES: NCc1ccc(F)c(Oc2ccc(N)cc2)c1F] | 4-(3- Aminomethyl- 2,6-difluoro- phenoxy)- phenylamine | 1H NMR (400 MHz, Me-d3- OD): 7.54-7.45 (1H, m), 7.40 (2H, d), 7.35-7.25 (1H, m), 7.20-7.11 (2H, m), 4.25 (2H, s). | Key Intermediate 5 |
| KI-6 | 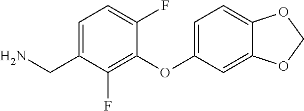 [SMILES: NCc1ccc(F)c(Oc2ccc3c(c2)OCO3)c1F] | 3-(Benzo[1,3] dioxol-5-yloxy)- 2,4-difluoro- benzylamine | 1H NMR (400 MHz, Me-d3- OD): 7.47-7.35 (1H, m), 7.29-7.17 (1H, m), 6.74 (1H, d), 6.59 (1H, d), 6.39 (1H, dd), 5.96 (2H, s), 4.22 (2H, s). [M − NH2] + 263 | Key Intermediate 6 |
| KI-7 | 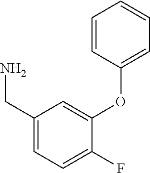 [SMILES: NCc1ccc(F)c(Oc2ccccc2)c1] | 4-Fluoro-3- phenoxy- benzylamine | 1H NMR (400 MHz, DMSO- d6): 7.44-7.27 (3H, m), 7.27- 7.19 (2H, m), 7.13 (1H, t), 6.97 (2H, d), 3.76 (2H, s). [MH]+ = 201 | Key Intermediate 7 |
| KI-8 | 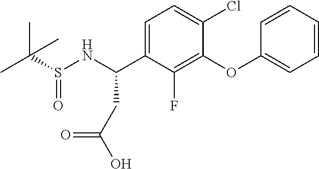 [SMILES: CC(C)(C)[S@@](=O)N[C@@H](CC(=O)O)c1ccc(Cl)c(Oc2ccccc2)c1F] | (S)-3-(4-Chloro- 2-fluoro- 3-phenoxy- phenyl)-3-((R)-2- methyl-propane- 2-sulfinylamino)- propionic acid | 1H NMR (400 MHz, Me-d3- OD): 7.44-7.36 (2H, m), 7.36-7.26 (2H, m), 7.12-7.02 (1H, m), 6.85 (2H, d), 5.06 (1H, t), 3.06 (1H, dd), 2.97 (1H, dd), 1.19 (9H, s). | Key Intermediate 8 |
| KI-9 | 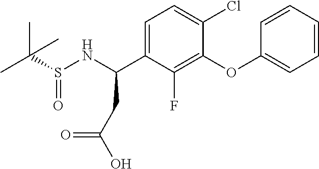 [SMILES: CC(C)(C)[S@@](=O)N[C@H](CC(=O)O)c1ccc(Cl)c(Oc2ccccc2)c1F] | (R)-3-(4-Chloro- 2-fluoro-3- phenoxy-phenyl)- 3-((R)-2-methyl- propane-2- sulfinylamino)- propionic acid | 1H NMR (400 MHz, Me-d3- OD): 7.44 (1H, dd), 7.40- 7.27 (3H, m), 7.12-7.02 (1H, m), 6.89 (2H, d), 5.07 (1H, dd), 2.93 (1H, dd), 2.83 (1H, dd), 1.23 (9H, s). | Key Intermediate 9 |
| KI-10 | 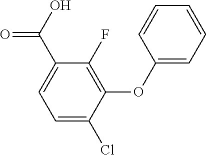 [SMILES: O=C(O)c1ccc(Cl)c(Oc2ccccc2)c1F] | 4-Chloro-2- fluoro-3- phenoxy- benzoic acid | ¹H NMR (400 MHz, DMSO- d6): 7.76 (1H, t), 7.56 (1H, d), 7.36 (2H, t), 7.11 (1H, t), 6.92 (2H, d). [MH]− = 265 | Key Intermediate 10 |
| KI-11 | 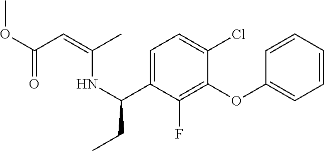 [SMILES: CC[C@@H](N/C(C)=C\C(=O)OC)c1ccc(Cl)c(Oc2ccccc2)c1F] | (Z)-3-[(R)-1-(4- Chloro-2-fluoro- 3-phenoxy- phenyl)- propylamino]- but-2-enoic acid methyl ester | 1H NMR (400 MHz, DMSO- d6): 8.98 (1H, d), 7.53 (1H, d), 7.36 (2H, t), 7.25 (1H, t), 7.11 (1H, t), 6.88 (2H, d), 4.80 (1H, q), 4.48 (1H, s), 3.54 (3H, s), 1.93-1.64 (5H, m), 0.97-0.77 (3H, m). | Key Intermediate 11 |
| KI-12 | 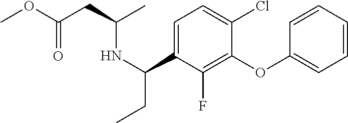 [SMILES: CC[C@@H](N[C@H](C)CC(=O)OC)c1ccc(Cl)c(Oc2ccccc2)c1F] | (R)-3-[(R)-1-(4- Chloro-2-fluoro- 3-phenoxy- phenyl)- propylamino]- butyric acid methyl ester | 1H NMR (400 MHz, Me-d3- OD): 7.45-7.36 (2H, m), 7.35-7.28 (2H, m), 7.06 (1H, t), 6.85 (2H, d), 4.00 (1H, dd), 3.63 (3H, s), 3.00-2.88 (1H, m), 2.53 (1H, dd), 2.29 (1H, dd), 1.91-1.75 (1H, m), 1.72-1.59 (1H, m), 1.06 (3H, | Key Intermediate 12 |
| | | | d), 0.84 (3H, t). | |
| | | | [M + H] + 380.0 | |
| KI-13 | 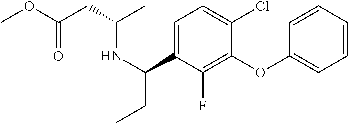 [SMILES: CC[C@@H](N[C@@H](C)CC(=O)OC)c1ccc(Cl)c(Oc2ccccc2)c1F] | (S)-3-[(R)-1-(4- Chloro-2-fluoro- 3-phenoxy- phenyl)- propylamino]- butyric acid methyl ester | 1H NMR (400 MHz, Me-d3- OD): 7.44-7.26 (4H, m), 7.07 (1H, t), 6.85 (2H, d), 4.03 (1H, dd), 3.63 (3H, s), 2.98-2.79 (1H, m), 2.42- 2.35 (2H, m), 1.93-1.77 (1H, m), 1.76-1.60 (1H, m), 1.07 (3H, d), 0.84 (3H, t). | Key Intermediate 13 |
| | | | [M + H] + 380.0 | |
| KI-14 | 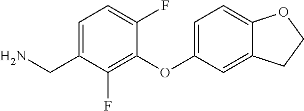 [SMILES: NCc1ccc(F)c(Oc2ccc3c(c2)CCO3)c1F] | 3-(2,3-Dihydro- benzofuran-5- yloxy)-2,4- difluoro- benzylamine | 1H NMR (400 MHz, Me-d3- OD): 7.45-7.33 (1H, m), 7.28-7.16 (1H, m), 6.88 (1H, s), 6.74-6.60 (2H, m), 4.55 (2H, t), 4.22 (2H, s), 3.18 (2H, t), 2.71 (3H, s). [M − NH2] + 261 | As Key Intermediate 6 |
| KI-15 | 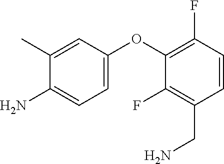 [SMILES: Cc1cc(Oc2c(F)ccc(CN)c2F)ccc1N] | 4-(3- Aminomethyl- 2,6-difluoro- phenoxy)-2- methyl- phenylamine | 1H NMR (400 MHz, Me-d3- OD): 7.31-7.20 (1H, m), 7.12-7.00 (1H, m), 6.72-6.63 (2H, m), 6.59 (1H, dd), 3.86 (2H, s), 2.14 (3H, s). | As Key Intermediate 5 using 5-fluoro-2- nitro toluene in step 1 |
| KI-16 | 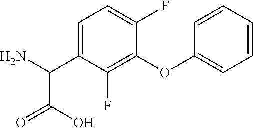 [SMILES: NC(C(=O)O)c1ccc(F)c(Oc2ccccc2)c1F] | Amino-(2,4- difluoro-3- phenoxy-phenyl)- acetic acid | 1H NMR (400 MHz, Me-d3- OD): 7.51-7.41 (1H, m), 7.41-7.25 (3H, m), 7.18-7.07 (1H, m), 6.96 (2H, d), 5.38 (1H, s). | Step 2 of example 46 |
| KI-17 | 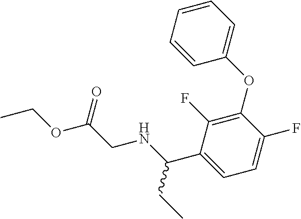 [SMILES: CCOC(=O)CNC(CC)c1ccc(F)c(Oc2ccccc2)c1F] | [1-(2,4-Difluoro- 3-phenoxy- phenyl)- propylamino]- acetic acid ethyl ester | 1H NMR (400 MHz, Me-d3- OD): 7.51-7.40 (1H, m), 7.40-7.29 (3H, m), 7.13 (1H, t), 6.97 (2H, d), 4.57 (1H, dd), 4.35-4.23 (2H, m), 4.04- 3.90 (2H, m), 2.31-2.19 (1H, m), 2.16-2.03 (1H, m), 1.30 (3H, t), 0.90 (3H, t). | As Example 42 using ethylbromoacetate |
| KI-18 | 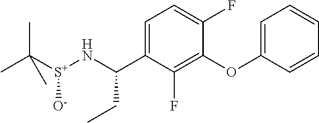 [SMILES: CC[C@H](N[S@+]([O-])C(C)(C)C)c1ccc(F)c(Oc2ccccc2)c1F] | (R)-2-Methyl- propane-2- sulfinic acid [(S)-1-(2,4- difluoro-3- phenoxy- phenyl)-propyl]- amide | [M + H] + 368 | As for Key Intermediate 1, step 5 but using (R)-tert- butylsulfinimide |
| KI-19 | 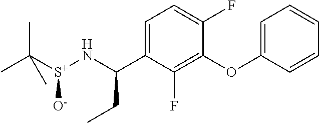 [SMILES: CC[C@@H](N[S@@+]([O-])C(C)(C)C)c1ccc(F)c(Oc2ccccc2)c1F] | (S)-2-Methyl- propane-2- sulfinic acid [(R)-1-(2,4- difluoro-3- phenoxy- phenyl)-propyl]- amide | [M + H] + 368 | Minor isomer isolated from Key Intermediate 1, step 5 |
| KI-20 | 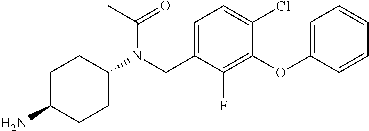 [SMILES: CC(=O)N(Cc1ccc(Cl)c(Oc2ccccc2)c1F)[C@H]1CC[C@H](N)CC1] | trans-N-(4- Amino- cyclohexyl)- N-(4-chloro-2- fluoro-3- phenoxy- benzyl)- acetamide | 1H NMR (Mixture of rotamers) (400 MHz, DMSO- d6): 7.99-7.72 (2H, m), 7.55- 7.30 (3H, m), 7.22-7.02 (2H, m), 6.94-6.83 (2H, m), 4.59 (0.8H, s), 4.45 (1.2H, s), 4.30-4.16 (0.4H, m), 3.79- 3.69 (0.6H, m), 2.99-2.87 | Atep 1 of example 273 |
| | | | (1H, m), 2.20 (1.6H, s), 1.98- | |
| | | | 1.87 (3.5H, m), 1.73 (1.2H, | |
| | | | d), 1.63-1.31 (4.7H, m). | |
| | | | [M + Na] + 413.0 | |
| KI-21 | 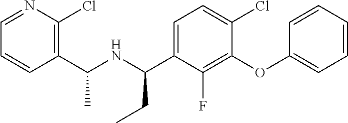 [SMILES: CC[C@@H](N[C@H](C)c1cccnc1Cl)c1ccc(Cl)c(Oc2ccccc2)c1F] | [(R)-1-(4-Chloro- 2-fluoro-3- phenoxy- phenyl)-propyl]- [(R)-1-(2-chloro- pyridin-3-yl)- ethyl]-amine | 1H NMR (400 MHz, Me-d3- OD): 8.45 (1H, dd), 8.18 (1H, dd), 7.63-7.52 (2H, m), 7.52-7.29 (3H, m), 7.16-7.06 (1H, m), 6.87 (2H, d), 4.75 (1H, q), 4.32 (1H, dd), 2.36- 2.23 (1H, m), 2.14-1.98 (1H, m), 1.71 (3H, d), 0.83 (3H, t). | Prepared in a manner analogous to example 5/6 using 1-(2-Chloro-pyridin- 3-yl)-ethanone and (R)-1- (4-chloro-2-fluoro-3- phenoxy-phenyl)- propylamine, followed by separation |
| | | | {M + H] + 419 | of diastereoisomers by |
| | | | | column chromatography. |
| KI-22 | 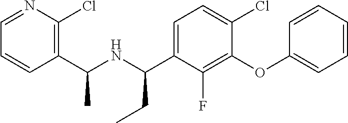 [SMILES: CC[C@@H](N[C@@H](C)c1cccnc1Cl)c1ccc(Cl)c(Oc2ccccc2)c1F] | [(R)-1-(4-Chloro- 2-fluoro-3- phenoxy- phenyl)-propyl]- [(S)-1-(2-chloro- pyridin-3-yl)- ethyl]-amine | 1H NMR (400 MHz, Me-d3- OD): 8.45 (1H, dd), 8.13 (1H, dd), 7.62-7.51 (2H, m), 7.47 (1H, dd), 7.41-7.30 (2H, m), 7.17-7.07 (1H, m), 6.89 (2H, d), 4.74 (1H, q), 4.63 (1H, dd), 2.34-2.21 (1H, m), 2.19-2.05 (1H, m), 1.75 (3H, | As for KI-21 |
| | | | d), 0.88 (3H, t). | |
| | | | {M + H] + 419 | |
| KI-23 | 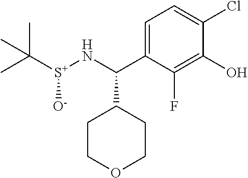 [SMILES: CC(C)(C)[S@@+]([O-])N[C@H](c1ccc(Cl)c(O)c1F)C1CCOCC1] | (R)-2-Methyl- propane-2- sulfinic acid [(S)-(4-chloro-2- fluoro-3- hydroxy-phenyl)- (tetrahydro- pyran-4-yl)- methyl]-amide | [M + H] + 364/366 | Prepared as for Example 75 step 1, but using (R)-2- methyl-propane-2-sulfinic acid 1-(tetrahydro-pyran-4- yl)-meth-(E)-ylideneamide (prepared from tetrahydropyranyl-4- carboxaldehyde in a manner analogous to example 61, step 2) |
| KI-24 | 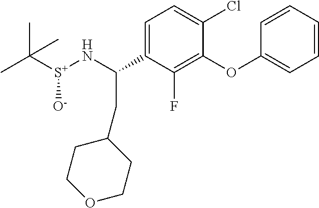 [SMILES: CC(C)(C)[S@@+]([O-])N[C@@H](CC1CCOCC1)c1ccc(Cl)c(Oc2ccccc2)c1F] | (R)-N-[(S)-1-(4- Chloro-2-fluoro- 3-phenoxy- phenyl)-2- (tetrahydro- pyran-4-yl)- ethyl]-2,2- dimethyl- propane- sulfinamide | 1H NMR (400 MHz, Me-d3- OD): 7.45-7.26 (4H, m), 7.08 (1H, t), 6.84 (2H, d), 4.76 (1H, t), 3.97-3.82 (2H, m), 3.45-3.34 (2H, m), 2.01-1.88 (1H, m), 1.83-1.71 (1H, m), 1.71-1.55 (3H, m), 1.41-1.25 (2H, m), 1.18 (9H, s). [M + H] + 454.0 | Example 276 |
| KI-25 | 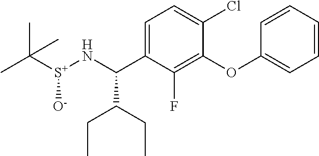 [SMILES: CCC(CC)[C@H](N[S@+]([O-])C(C)(C)C)c1ccc(Cl)c(Oc2ccccc2)c1F] | (R)-N-[(S)-1-(4- Chloro-2-fluoro- 3-phenoxy- phenyl)-2-ethyl- butyl]-2,2- dimethyl- propane- sulfinamide | 1H NMR (400 MHz, Me-d3- OD): 7.38 (1H, dd), 7.35-7.25 (3H, m), 7.08 (1H, t), 6.82 (2H, d), 4.55 (1H, dt), 1.90- 1.77 (1H, m), 1.77-1.63 (1H, m), 1.63-1.48 (1H, m), 1.44- 1.29 (1H, m), 1.24-1.17 (1H, m), 1.14 (9H, d), 0.95 (3H, t), 0.85 (3H, t). [M + H] + 426.0 | As for Example 276 step 1 using 3-pentyl magnesium bromide in step 1. |
| KI-26 | 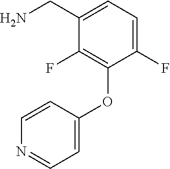 [SMILES: NCc1ccc(F)c(Oc2ccncc2)c1F] | 2,4-Difluoro-3- (pyridin-4-yloxy)- benzylamine | [M + H] + 237 | As for KI-6 using pyridin- 4-yl boronic acid in step 1. |
| KI-27 | 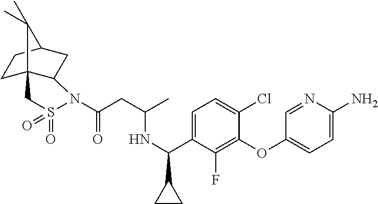 | | LCMS: 591.4 (MH⁺). | From Example 397 using General Method 1 below. Stirred for 4 days. Purified on silica (60 g-1:1 up to 2:1 EtOAc/heptanes) to give (380 mg, 44% yield). |
| KI-28 | 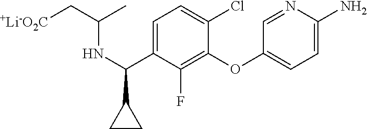 | | LCMS: 394.2 (MH⁺). | From KI-27 using General Method 2 below. |
| KI-29 | 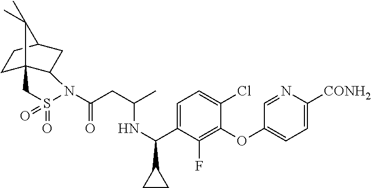 | | LCMS: 619.5 (MH⁺). | From Example 398 using General Method 1 below. Stirred for 4 days. Purified on silica (40 g-1:1 up to 2:1 EtOAc/heptanes) to give 492 mg (55% yield). |
| KI-30 | 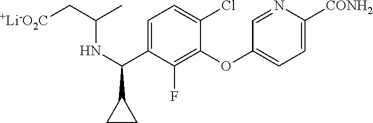 | | LCMS: 422.2 (MH⁺). | From KI-29 using General Method 2 below. |

### GENERAL METHODS

### General Method 1

### Conversion of a Compound of Formula (1) Wherein R⁰ and R² are Both Hydrogen to a Camphor Sultam Adduct of Formula

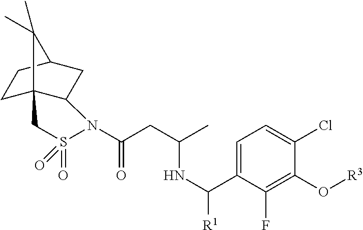

To the hydrochloride salt (0.5 mmol, 1.0 eq) of the benzylamine compound of formula (1) (R⁰ and R² are both hydrogen) was added DCM (5 ml) and sat. NaHCO₃ solution (5 ml) [pH checked >7]. The organic layer was separated off and concentrated in vacuo. To the free amine was added THF (1 ml), lithium perchlorate (74.5 mg, 0.7 mmol, 1.4 eq) and (R)-(−)-(2-butenoyl)-2,10-camphorsultam (170 mg, 0.6 mmol, 1.2 eq). The reaction was stirred at 20° C. for the specified time. EtOAc (10 ml) was added and the organic layer washed with water (10 ml) then sat. brine (10 ml). The organic layer was dried, filtered and concentrated in vacuo. The material was purified by column chromatography on silica (EtOAcheptanes).

### General Method 2

### Hydrolysis of a Camphor Sultam Adduct to Give a Lithium Carboxylate Salt of the Formula

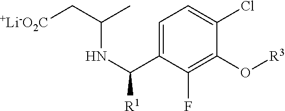

The Camphor sultam adduct (0.5 mmol, 1.0 eq) prepared by General Method 1 was dissolved in THF (20 vols) and a 1M aqueous solution of LiOH (1.0 ml, 1.0 mmol, 2.0 eq) was added. The mixture was stirred overnight and then the solvent removed in vacuo. A THF strip was utilized to remove any residual water.

### General Method 3

### Conversion of the Lithium Carboxylate Salt Prepared by General Method 2 to the Corresponding Amide of the Formula

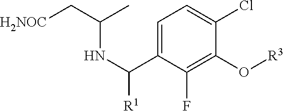

To the lithio salt (0.5 mmol) dissolved in DMF (10 ml) was charged sequentially NH₄Cl (133 mg, 2.5 mmol, 5 eq), triethylamine (488 μl, 3.5 mmol, 7 eq) and then HATU (285 mg, 0.75 mmol, 1.5 eq); the mixture was stirred for 5-24 hours at 20° C. Additional HATU was charged as required. EtOAc (20 ml) was added and the organic layer washed with water (10 ml), 10% LiCl (10 ml) and sat. brine (10 ml) before being dried, filtered and concentrated in vacuo. The material was purified by column chromatography on silica (60-100 equivalents) eluting with MeOHNH₃ in either DCM or EtOAc. [Normal grade silica: ZEOprep 6040-63 microns (Apollo Scientific); TLC grade silica: silica gel 60 H, 90%<55 μm (Merck)].

The hydrochloride salts were formed by dissolving the free base in either Et₂O, EtOAc or DCM and addition of 2 eq HCl in EtOAc (2M) or Et₂O (2M). The solid was isolated by filtration and dried using a vacuum oven at 40-50° C.

### General Method 4

### Reduction of an Aromatic Nitro Substituent to an Aromatic Amino Substituent

To a solution of a nitro compound (0.098 mmol, 1 eq) in MeOH (2.5 ml) was added Fe powder (54 mg, 0.98 mmol, 10 eq) and NH₄Cl (52 mg, 0.98 mmol, 10 eq) dissolved in water (1.8 ml). The reaction was stirred under N₂ at 60° C. for 1 h. The reaction was filtered through Celite, the pad was washed with MeOH (2×25 ml) and the filtrate was concentrated in vacuo. The residue was purified via chromatography (silica, 3 g) eluting with 0.2% 0.88 ammonia9.8% MeOH90% EtOAc. The residue was dissolved in Et₂O (3 ml) and EtOAc (1.5 ml) and to the solution was added 2.1 M HCl in EtOAc (0.5 ml). The white precipitate was filtered, washed with Et₂O (2 ml) and dried in an oven at 40° C. overnight under vacuum.

### EXAMPLES

### Example 1

### 1-(2,4-Difluoro-3-phenoxy-phenyl)-2-methyl-propylamine. hydrochloride

### Step 1

A mixture of 2,4-difluoro-3-hydroxybenzaldehyde (1.67 g, 10.5 mmol), phenyl boronic acid (3.2 g, 26.4 mmol), copper (II) acetate (2.4 g, 13.7 mmol), pyridine (1.0 g, 10.5 mmol), pyridine-N-oxide (4.25 ml, 52.5 mmol) and 4 Å molecular sieves (2.5 g) in DCM (50 ml) was stirred at room temperature for 48 hours. The reaction was quenched with sat. sodium hydrogen carbonate and the resulting suspension filtered through celite. The layers were separated and the aqueous fraction further extracted with DCM. The combined organic fractions were dried over sodium sulfate, filtered and concentrated. The residue was purified by column chromatography. Eluting with 20% DCM in petrol afforded 2,4-Difluoro-3-phenoxybenzaldehyde (2.26 g) as an impure, colourless oil, which was used without further purification.

### Step 2

2,4-Difluoro-3-phenoxybenzaldehyde (2.2 g) was reacted with tert-butyl sulfinimide and titanium (IV) ethoxide as described in the synthesis of Key Intermediate 1, step 4 to yield 2-methyl-propane-2-sulfinic acid 1-(2,4-difluoro-3-phenoxy-phenyl)-meth-(E)-ylideneamide (1.79 g) as an off-white solid. MS: [M+H]⁺338.

### Step 3

To a cooled solution (−78° C.) of 2-methyl-propane-2-sulfinic acid 1-(2,4-difluoro-3-phenoxy-phenyl)-meth-(E)-ylideneamide (100 mg, 0.3 mmol) in THF (5 ml) was added dropwise iso-propyl lithium (0.57 ml of a 0.7 M solution in pentanes, 0.4 mmol) maintaining a temperature below −68° C. The resulting solution was stirred at −78° C. for 1 hour, then partitioned between sat. ammonium chloride and DCM. The organic fractions were dried over sodium sulfate, filtered and concentrated. The residue was redissolved in methanol (1.5 ml) and HCl (0.15 ml of a 4M solution in dioxane) was added. After stirring at room temperature for 1 hour, the reaction mixture was evaporated to dryness and triturated with diethyl ether to give the title compound (64 mg) as a white solid.

### Example 3

### 1-(2,4-Difluoro-3-phenoxy-phenyl)-propylamine. hydrochloride

Ethyl magnesium bromide (0.23 ml of a 3 M solution in diethyl ether, 0.69 mmol) was added to a solution of dimethyl zinc (0.76 ml of a 1 M solution in heptanes, 0.76 mmol) in THF (1 ml). The mixture was stirred at room temperature for 15 mins, then transferred via cannula to a cooled solution (−78° C.) of 2-methyl-propane-2-sulfinic acid 1-(2,4-difluoro-3-phenoxy-phenyl)-meth-(E)-ylideneamide (prepared as described in Example 1) (150 mg, 0.44 mmol) in THF (5 ml). The resulting solution was stirred at −78° C. for 1 hour, ethyl magnesium bromide (0.23 ml of a 3 M solution in diethyl ether, 0.67 ml) was added and the reaction stirred 1 hour further at −78° C. The reaction was quenched with sat. ammonium chloride, allowed to warm to room temperature and extracted with DCM. The organic fractions were dried over sodium sulfate, filtered and concentrated. The residue was redissolved in methanol (2 ml) and HCl (2 ml of a 4M solution in dioxane) was added. After stirring at room temperature for 1 hour, the reaction mixture was evaporated to dryness and triturated with diethyl ether to give 1-(2,4-difluoro-3-phenoxy-phenyl)-propylamine hydrochloride (110 mg) as a white solid.

### Examples 5 and 6

### Trans-N-[1′-(2,4-difluoro-3-phenoxy-phenyl)-propyl]-cyclohexane-1,4-diamine and cis-N-[1-(2,4-Difluoro-3-phenoxy-phenyl)-propyl]-cyclohexane-1,4-diamine dihydrochloride

### Step 1

Triethylamine (0.04 ml, 0.29 mmol) was added to a mixture of 1-(2,4-difluoro-3-phenoxy-phenyl)-propylamine hydrochloride (80 mg, 0.27 mmol) and (4-oxo-cyclohexyl)-carbamic acid tert-butyl ester (57 mg, 0.27 mmol) in DCE (4 ml), followed by glacial acetic acid (0.03 ml, 0.53 mmol) and sodium triacetoxyborohydride (113 mg, 0.53 mmol). The resulting mixture was stirred at room temperature for 2 hours, then poured into 1 M sodium hydroxide and extracted into DCM. The residue was purified preparative hplc to afford the trans-substituted {4-[1-(2,4-difluoro-3-phenoxy-phenyl)-propylamino]-cyclohexyl}-carbamic acid tert-butyl ester (43 mg) as a white solid. MS: [M+H]⁺461. Further elution yielded the cis-substituted {4-[1-(2,4-difluoro-3-phenoxy-phenyl)-propylamino]-cyclohexyl}-carbamic acid tert-butyl ester (51 mg) as a colourless gel. MS: [M+H]⁺461.

### Step 2

Trans {-[1-(2,4-difluoro-3-phenoxy-phenyl)-propylamino]-cyclohexyl}-carbamic acid tert-butyl ester (51 mg, 0.09 mmol) was dissolved in a 4M solution of HCl in ethyl acetate (3 ml) and stirred for 3 hours. The resulting suspension was filtered and the solid washed with ethyl acetate and dried to give the title compound (33 mg) as a white solid. The cis derivative was deprotected and isolated in an analogous manner.

### Example 7

### (4-Aminomethyl-pyrimidin-2-yl)-[(R)-1-(4-chloro-2-fluoro-3-phenoxy-phenyl)-propyl]-amine hydrochloride

### Step 1

A mixture of 2-chloro-pyrimidine-4-carbonitrile (prepared analogously to WO2010025553 page 55 step 7, 110 mg, 0.79 mmol), (R)-1-(4-chloro-2-fluoro-3-phenoxy-phenyl)-propylamine (prepared in an analogous fashion to Key Intermediate 1) (249 mg, 0.79 mmol), potassium carbonate (450 mg, 3.3 mmol) and dimethylformamide (3 ml) was heated to 100° C. overnight. The reaction mixture was allowed to cool, ethyl acetate was added and the mixture was washed with water, 10% aqueous lithium chloride and saturated brine. The organic layer was dried (magnesium sulphate) and concentrated, purified by column chromatography, eluting with 5-30% ethyl acetate in petrol to furnish 2-[(R)-1-(4-chloro-2-fluoro-3-phenoxy-phenyl)-propylamino]-pyrimidine-4-carbonitrile (132 mg) as an oil. MS: [M+H]⁺383385

### Step 2

A mixture of 2-[(R)-1-(4-chloro-2-fluoro-3-phenoxy-phenyl)-propylamino]-pyrimidine-4-carbonitrile (132 mg, 0.35 mmol) and Raney Nickel (catalytic amount) in ethyl acetate (4 ml) and ammonia in methanol (7N, 4 ml) was stirred at room temperature under a hydrogen atmosphere overnight. The mixture was then filtered through GF-A paper under suction and concentrated. The residue was purified by preparative HPLC and salted using 2N hydrochloric acid in ethyl acetate to furnish (4-aminomethyl-pyrimidin-2-yl)-[(R)-1-(4-chloro-2-fluoro-3-phenoxy-phenyl)-propyl]-amine hydrochloride as a white solid.

### Example 8

### (5-Aminomethyl-pyrimidin-2-yl)-[(R)-1-(4-chloro-2-fluoro-3-phenoxy-phenyl)-propyl]-amine hydrochloride

### Step 1

(5-Bromo-pyrimidin-2-yl)-[(R)-1-(4-chloro-2-fluoro-3-phenoxy-phenyl)-propyl]-amine was prepared analogously to Example 7 Step 1 using 5-bromo-2-chloropyrimidine. MS: [M+H]⁺436438

### Step 2

2-[(R)-1-(4-Chloro-2-fluoro-3-phenoxy-phenyl)-propylamino]-pyrimidine-5-carbonitrile was prepared using the route analogous to that described in US20090062541. MS: [M+H]⁺383385

### Step 3

2-[(R)-1-(4-Chloro-2-fluoro-3-phenoxy-phenyl)-propylamino]-pyrimidine-5-carbonitrile was reduced using the procedure in Example 7 Step 2 to furnish (5-aminomethyl-pyrimidin-2-yl)-[(R)-1-(4-chloro-2-fluoro-3-phenoxy-phenyl)-propyl]-amine hydrochloride as a white solid

### Example 9

### (S)—N-(2-Amino-ethyl)-2-[(R)-1-(4-chloro-2-fluoro-3-phenoxy-phenyl)-propylamino]-propionamide dihydrochloride

### Step 1

(R)-1-(4-chloro-2-fluoro-3-phenoxy-phenyl)-propylamine (prepared in an analogous fashion to Key Intermediate 1) (50 mg, 0.16 mmol) was alkylated using (R)-2-trifluoromethane-sulfonyloxy-propionic acid methyl ester (0.95 ml, 0.95 mmol) in an analogous fashion to US20060105964 Example 1 Step 1 furnishing (S)-2-[(R)-1-(4-chloro-2-fluoro-3-phenoxy-phenyl)-propylamino]-propionic acid methyl ester as an oil (77 mg). MS: [M+H]⁺366368

### Step 2

(S)—N-(2-Amino-ethyl)-2-[(R)-1-(4-chloro-2-fluoro-3-phenoxy-phenyl)-propylamino]-propionamide dihydrochloride was prepared by hydrolysis, amide bond formation (using (2-amino-ethyl)-carbamic acid tert-butyl ester) and deprotection according to methods in Example 131 Step 2 and Example 223.

### Example 13

### C-(2,4-Difluoro-3-phenoxy-phenyl)-C-(1,2,3,6-tetrahydro-pyridin-4-yl)-methylamine. dihydrochloride

To a solution of 2-methyl-propane-2-sulfinic acid 1-(2,4-difluoro-3-phenoxy-phenyl)-meth-(E)-ylideneamide (prepared as described in Example 1) (200 mg, 0.59 mmol), bis(acetonitrile)(1,5-cyclooctadiene)rhodium(I)tetrafluoroborate (22 mg, 0.06 mmol) and (N-Boc)-1,2,3,6-tetrahydropyridine-4-boronic acid pinacol ester (180 mg, 0.59 mmol) in dioxane (2.5 ml) were added triethylamine (0.17 ml, 1.18 mmol) and water (2.5 ml). The resulting mixture was stirred overnight at room temperature and partitioned between water and DCM. The aqueous fraction was further extracted with DCM and the combined organic fractions were dried over sodium sulfate, filtered, concentrated and purified by column chromatography, eluting with 30-40% ethyl acetate in petrol. The residue (90 mg) was dissolved in methanol (3 ml) and HCL (1 ml of a 4M solution in dioxane) was added. After stirring for 1 hour at room temperature, the solution was concentrated and the residue triturated with diethyl ether to yield the title compound as an off-white solid.

### Example 14

### C-(2,4-Difluoro-3-phenoxy-phenyl)-C-piperidin-4-yl-methylamine. dihydrochloride

A suspension of C-(2,4-difluoro-3-phenoxy-phenyl)-C-(1,2,3,6-tetrahydro-pyridin-4-yl)-methylamine (30 mg, 0.1 mmol) and PdC (30 mg) in methanol (2 ml) was stirred under a hydrogen atmosphere for 2 hours, then filtered through celite. The filtrate was concentrated and the residue triturated with a small volume of methanol to afford the title compound as a white solid.

### Examples 15A and 15B

### 1-(2,4-difluoro-3-phenoxy-phenyl)-2-nitro-ethylamine (Compound 15A) and

### 1-(2,4-Difluoro-3-phenoxy-phenyl)-ethane-1,2-diamine (Compound 15B)

### Step 1

Tetrabutyl ammonium fluoride (1.2 ml of a 1M solution in THF, 1.2 mmol) was added to a solution of 2-methyl-propane-2-sulfinic acid 1-(2,4-difluoro-3-phenoxy-phenyl)-meth-(E)-ylideneamide (prepared as described in Example 1) (400 mg, 1.2 mmol), in nitromethane (3 ml). The reaction was stirred for 40 mins at room temperature, then filtered through a short pad of silica, eluting with ethyl acetate. The solvent was evaporated and the residue purified by column chromatography, eluting with 30-40% ethyl acetate in petrol to yield 1-(2,4-difluoro-3-phenoxy-phenyl)-2-nitro-ethylamine (Compound 15A) (240 mg) as an off-white solid. MS: [M+H]⁺399. Further elution afforded the other diastereomer (80 mg) as an off-white foam. MS: [M+H]⁺399. The first diastereomer (76 mg, 0.19 mmol) was dissolved in methanol (3 ml) and HCl (2 ml of a 4M solution in dioxane) was added. After stirring for 1 hour, the solution was concentrated and the residue triturated with diethyl ether to give the product (53 mg) as a white solid.

### Step 2

1-(2,4-Difluoro-3-phenoxy-phenyl)-2-nitro-ethylamine (43 mg, 0.16 mmol) was dissolved in methanol (2 ml). PdC (40 mg) and HCl (1 ml of a 4 M solution in dioxane, 4 mmol) were added and the resulting suspension was stirred under a hydrogen atmosphere overnight. The mixture was filtered through celite and the filtrate was concentrated and triturated with diethyl ether to yield the product, 1-(2,4-difluoro-3-phenoxy-phenyl)-ethane-1,2-diamine (Compound 15B), (35 mg) as a white solid.

### Example 16

### [1-(2,4-Difluoro-3-phenoxy-phenyl)-3-methyl-butyl]-methyl-amine. hydrochloride

### Step 1

A solution of 1-(2,4-difluoro-3-phenoxy-phenyl)-3-methyl-butylamine (prepared analogously to Example 1) (70 mg, 0.24 mmol) and ethyl chloroformate (0.03 ml, 0.26 mmol) in DCM (4 ml) was cooled to −30° C., before triethylamine (0.04 ml, 0.26 mmol) was added dropwise. The reaction was allowed to warm to room temperature and stirred for 1 hour before being quenched with 1M HCl. The aqueous layer was extracted with DCM and the combined organics were washed with sat. sodium hydrogen carbonate, dried over sodium sulfate, filtered and concentrated. The product, [1-(2,4-difluoro-3-phenoxy-phenyl)-3-methyl-butyl]-carbamic acid ethyl ester, was used in the next step without further purification.

### Step 2

Lithium aluminium hydride (0.5 ml of a 2M solution in THF) was added to a solution of [1-(2,4-difluoro-3-phenoxy-phenyl)-3-methyl-butyl]-carbamic acid ethyl ester (0.24 mmol, assumed) in THF (5 ml) at 0° C. The reaction was allowed to warm to room temperature and stirred for 2 hours. The reaction was cooled back to 0° C. and diethyl ether (5 ml) was added, followed by water (20 ml), 15% sodium hydroxide (36 ml) and water (40 ml). The resulting suspension was filtered and washed with hot ethyl acetate. The filtrate was concentrated and the residue purified by preparative hplc to generate the title compound (12 mg) as a solid.

### Example 19

### 1-(2,4-Difluoro-3-phenoxy-phenyl)-N*2*-isopropyl-ethane-1,2-diamine. dihydrochloride

### Step 1

2-Methyl-propane-2-sulfinic acid [1-(2,4-difluoro-3-phenoxy-phenyl)-2-nitro-ethyl]-amide (prepared as described in Example 15) (827 mg, 2.07 mmol) was dissolved in methanol (5 ml). HCl (5 ml of a 4M solution in dioxane) was added and the resulting solution stirred at room temperature for 1 hour. The mixture was concentrated and triturated with diethyl ether and the solid redissolved in THF (10 ml). Di-tert-butyl dicarbonate (327 mg, 3.11 mmol) was added, followed by 1M sodium hydrogen carbonate (6.2 ml, 6.2 mmol) and the resulting mixture was stirred at room temperature for 3.5 hours. The mixture was extracted with DCM and the organic fractions dried over sodium sulfate, filtered and evaporated. The residue was purified by column chromatography. Elution with 0-10% ethyl acetate in petrol afforded [1-(2,4-difluoro-3-phenoxy-phenyl)-2-nitro-ethyl]carbamic acid tert-butyl ester (500 mg) as a white solid. MS: [M+Na]⁺417.

### Step 2

[1-(2,4-difluoro-3-phenoxy-phenyl)-2-nitro-ethyl]carbamic acid tert-butyl ester (500 mg, 1.26 mmol) was dissolved in methanol (5 ml) and THF (5 ml). PdC was added and the suspension shaken overnight under a hydrogen atmosphere before being filtered. The filtrate was concentrated in vacuo to give [1-(2,4-difluoro-3-phenoxy-phenyl)-2-amino-ethyl]-carbamic acid tert-butyl ester (390 mg) as a grey powder which was used without further purification.

### Step 3

[1-(2,4-difluoro-3-phenoxy-phenyl)-2-amino-ethyl]carbamic acid tert-butyl ester (80 mg, 0.22 ml) was reductively aminated with acetone in a manner analogous to that described in Example 56, step 1. The product was dissolved in methanol (2 ml) and HCl (2 ml of a 4M solution in dioxane) and stirred for 1 hour at room temperature, before being concentrated and triturated with diethyl ether to afford the title compound (20 mg) as a white solid.

### Example 20

### 3-Amino-3-(2,4-difluoro-3-phenoxy-phenyl)-N-pyridin-4-yl-propionamide. dihydrochloride

### Step 1

Anhydrous methyl acetate (0.07 ml) was added to a cooled solution of sodium hexadimethylsilazide (0.9 ml of a 1M solution in THF, 0.9 mmol) in diethyl ether (5 ml) at −78° C. under an inert atmosphere. The resulting solution was stirred 1 hour further at this temperature and a solution of 2-methyl-propane-2-sulfinic acid 1-(2,4-difluoro-3-phenoxy-phenyl)-meth-(E)-ylideneamide (prepared as described in Example 1) (200 mg, 0.59 mmol) in diethyl ether (5 ml) was added. The reaction was stirred for 4 hours at −78° C., quenched with sat. ammonium chloride and allowed to warm to room temperature. The layers were separated and the organic fraction concentrated. The residue was taken up in 1M lithium hydroxide (2 ml), THF (1 ml) and methanol (1 ml) then stirred at room temperature overnight. 10% HCl was added until a suspension appeared and the mixture was extracted with ethyl acetate. The organic fractions were washed with 5% HCl and brine, dried over sodium sulfate, filtered and concentrated to yield 3-(2,4-difluoro-3-phenoxy-phenyl)-3-(2-methyl-propane-2-sulfinylamino)-propionic acid (200 mg) as a colourless powder which was used without further purification. MS: [M+H]⁺398.

### Step 2

A solution of 3-(2,4-difluoro-3-phenoxy-phenyl)-3-(2-methyl-propane-2-sulfinylamino)-propionic acid (100 mg, 0.25 mmol), 1-ethyl-3-(3-dimethylaminopropyl)carbodiimide (58 mg, 0.3 mmol), 1-hydroxybenzotriazole (40 mg, 0.3 mmol) and 4-aminopyridine (47 mg, 0.5 mmol) in DMF (3 ml) was stirred at room temperature for 48 hours. The DMF was evaporated and the residue partitioned between water and ethyl acetate. The organic fractions were washed with sat. sodium hydrogen carbonate, dried over sodium sulfate, filtered and evaporated to dryness. The residue was subjected to column chromatography. Elution with 5% methanol in DCM afforded 3-(2,4-difluoro-3-phenoxy-phenyl)-3-(2-methyl-propane-2-sulfinylamino)-N-pyridin-4-yl-propionamide (32 mg) as an impure solid, which was used without further purification. MS: [M+H]⁺474.

### Step 3

Crude 3-(2,4-difluoro-3-phenoxy-phenyl)-3-(2-methyl-propane-2-sulfinylamino)-N-pyridin-4-yl-propionamide (32 mg, 0.07 mmol) was dissolved in methanol (2 ml) and HCl (2 ml of a 4M solution in dioxane) was added. The mixture was stirred for 30 min, concentrated in vacuo and triturated with diethyl ether to afford the title compound (27 mg) as a white solid.

### Example 28

### (R)-{3-[(R)-1-(4-Chloro-2-fluoro-3-phenoxy-phenyl)-propylamino]-butyrylamino}-acetic acid methyl ester. hydrochloride

### Step 1

A solution of 3-[(R)-1-(4-chloro-2-fluoro-3-phenoxy-phenyl)-propylamino]-butyric acid methyl ester (Example 131 Step 1) (743 mg, 1.96 mmol) and lithium hydroxide (2.74 ml of a 1M aqueous solution, 2.74 mmol) in methanol (10 ml) was stirred at room temperature overnight, then concentrated.

### Step 2

A 100 mg portion of the residue was taken up in DMF (2 ml) and diisopropylethylamine (0.26 ml, 1.5 mmol) and glycine methyl ester hydrochloride (135 mg, 1.07 mmol) were added followed by 2-(1H-7-azabenzotriazol-1-yl)-1,1,3,3-tetramethyluronium hexafluorophosphate (122 mg, 0.32 mmol). The reaction mixture was stirred for 1 hour at room temperature before 2-(1H-7-azabenzotriazol-1-yl)-1,1,3,3-tetramethyluronium hexafluorophosphate (122 mg, 0.32 mmol) was added and the reaction stirred 1 hour further. The mixture was concentrated, then partitioned between water and chloroform. The organic fractions were dried over sodium sulfate, filtered and concentrated. The residue was subjected to preparative hplc and subsequent HCl salt formation to yield the (R,R) isomer (12 mg) as a white solid. Further elution and subsequent HCl salt formation yielded the (R,S) isomer (19 mg) also as a white solid.

### Example 39

### Allyl-[1-(2,4-difluoro-3-phenoxy-phenyl)-propyl]-amine. hydrochloride

### Step 1

1-(2,4-difluoro-3-phenoxy-phenyl)-propylamine hydrochloride (prepared as described in Example 3, step 1) (400 mg, 1.33 mmol) was dissolved in chloroform and cooled to 0° C. before triethylamine (0.41 ml, 2.93 mmol) and di-tert-butyl dicarbonate (349 mg, 1.6 mmol) were added. The reaction was allowed to warm to room temperature and stirred overnight. Water was added and the layers separated. The aqueous portion was further extracted with DCM and the combined organic fractions were dried over magnesium sulfate, filtered and evaporated to afford and N-Boc-1-(2,4-difluoro-3-phenoxy-phenyl)-propylamine as an impure solid, which was used without further purification. MS: [M+Na]⁺386.

### Step 2

Allyl bromide (0.01 ml, 0.14 mmol) was added to a suspension of sodium hydride (5.6 mg of a 60% suspension in mineral oils, 0.14 mmol) and N-Boc-1-(2,4-difluoro-3-phenoxy-phenyl)-propylamine (50 mg, 0.14 mmol) in THF (3 ml) at 0° C. The reaction was stirred for 1 hour at 0° C., 1 hour at room temperature and overnight at 60° C. Allyl bromide (0.01 ml, 0.14 mmol) and sodium hydride (5.6 mg of a 60% suspension in mineral oils, 0.14 mmol) were added and the reaction mixture heated for a further 1 hour at 70° C. The mixture was cooled and partitioned between water and ethyl acetate. The combined organic fractions were washed with brine, dried over magnesium sulfate, filtered and concentrated. The crude residue was taken up in HCl (4 ml of a 4M solution in ethyl acetate), stirred for 2 hours at room temperature, concentrated and triturated with diethyl ether to afford the title compound (14 mg) as a solid.

### Example 42

### [1-(2,4-Difluoro-3-phenoxy-phenyl)-propyl]-(2-methoxy-ethyl)-amine. hydrochloride

1-Bromo-2-methoxyethane (36 mg, 0.26 mmol) was added to a suspension of 1-(2,4-difluoro-3-phenoxy-phenyl)-propylamine hydrochloride (prepared as described in Example 3) (80 mg, 0.26 mmol) and potassium carbonate (84 mg, 0.52 mmol) in THF (2 ml). The reaction mixture was heated to 60° C. for 1 hour. DMSO (1 ml) was added and the reaction was heated for a further 6 hours at 80° C. The mixture was partitioned between water and ethyl acetate and the organic fractions were dried over magnesium sulfate, filtered and concentrated. The residue was purified by preparative hplc to yield the title compound (20 mg) as a white solid.

### Example 45

### 2-[1-(2,4-Difluoro-3-phenoxy-phenyl)-propylamino]-ethanol. hydrochloride

### Step 1

Ethyl bromoacetate (0.033 ml, 0.26 mmol) and potassium iodide (3 mg, cat.) were added to a suspension of 1-(2,4-difluoro-3-phenoxy-phenyl)-propylamine hydrochloride (prepared as described in Example 3) (80 mg, 0.26 mmol) and di-iso-propylethylamine (0.1 ml, 0.52 mmol) in THF (2 ml). The reaction was stirred for 3 hours at room temperature then at 60° C. for 2 hours. The reaction mixture was partitioned between sat. sodium hydrogen carbonate and ethyl acetate and the organic fractions were dried over magnesium sulfate, filtered and concentrated. The residue was purified by preparative hplc to give [1-(2,4-difluoro-3-phenoxy-phenyl)-propylamino]-acetic acid ethyl ester (30 mg) as a solid. MS: [M+Na]⁺372.

### Step 2

Lithium aluminium hydride (0.04 ml of a 2M solution in THF, 0.08 mmol) was added to a solution of [1-(2,4-difluoro-3-phenoxy-phenyl)-propylamino]-acetic acid ethyl ester (30 mg, 0.08 mmol) in THF (1 ml) at 0° C. The reaction was stirred for 1 hour at 0° C., lithium aluminium hydride (0.04 ml of a 2M solution in THF, 0.08 mmol) was added and the mixture allowed to warm to room temperature and stirred 1 hour further. The reaction mixture was partitioned between 1M sodium hydroxide and ethyl acetate. The organic fractions were dried over magnesium sulfate, filtered and concentrated and the residue purified by preparative hplc to afford the title compound (9 mg) as an off-white solid.

### Example 46

### 2-Amino-2-(2,4-difluoro-3-phenoxy-phenyl)-ethanol. hydrochloride

### Step 1

2,4-difluoro-3-phenoxybenzaldehyde (2 g, 3.54 mmol) (prepared as described in Example 1, Step 1) was dissolved in THF (30 ml) and cooled to −40° C. Lithium hexamethyldisilazide (10.25 ml of a 1M solution in THF, 10.25 mmol) was added dropwise. The resulting solution was allowed to warm to room temperature and stirred for 4 hours before acetone cyanohydrin (1.56 ml, 17.1 mmol) was added. After stirring at room temperature overnight, the mixture was partitioned between water and ethyl acetate. The combined organic fractions were dried over sodium sulfate, filtered and concentrated. The residue was purified by column chromatography. Elution with 20-35% ethyl acetate in petrol gave amino-(2,4-difluoro-3-phenoxy-phenyl)-acetonitrile (940 mg) as an orange gum. MS: [M+H−NH₃]⁺244.

### Step 2

Amino-(2,4-difluoro-3-phenoxy-phenyl)-acetonitrile (233 mg, 0.90 mmol) was heated to reflux in 6N HCl for 3 hours. The solvent was evaporated and the residue azeotroped with toluene, then triturated with diethyl ether to give amino-(2,4-difluoro-3-phenoxy-phenyl)-acetic acid (262 mg) as an off-white solid. MS: [M+H]⁺280.

### Step 3

To a solution of amino-(2,4-difluoro-3-phenoxy-phenyl)-acetic acid (262 mg, 0.83 mmol) in methanol (8 ml), cooled to 0° C. was added thionyl chloride (0.18 ml, 2.5 mmol). The reaction was allowed to warm to room temperature and stirred overnight. The solvent was evaporated and the residue triturated with diethyl ether to afford amino-(2,4-difluoro-3-phenoxy-phenyl)-acetic acid methyl ester (211 mg) as an off-white solid. MS: [M+Na]⁺316.

### Step 4

To a solution of amino-(2,4-difluoro-3-phenoxy-phenyl)-acetic acid methyl ester (100 mg, 0.34 mmol) in methanol (5 ml) cooled to 0° C. was added sodium borohydride (130 mg, 3.4 mmol). The reaction was allowed to warm to room temperature and stirred for 2 hours before being quenched with 1M sodium hydroxide and extracted into DCM. The combined organic fractions were dried over sodium sulfate, filtered and evaporated and the residue subjected to column chromatography. Elution with 6% 2M NH₃ in methanol in DCM yielded the title compound (23 mg) as a white solid.

### Example 50

### C-(2,4-Difluoro-3-phenoxy-phenyl)-C-(4,5-dihydro-1H-imidazol-2-yl)-methylamine.dihydrobromide

### Step 1

Benzoyl chloride (550 mg, 3.2 mmol) and sodium hydrogen carbonate (450 mg, 5.4 mmol) were added to a solution of amino-(2,4-difluoro-3-phenoxy-phenyl)-acetonitrile (prepared as described in Example 46, step 1) (700 mg, 2.7 mmol) in acetonewater (1:1, 10 ml). The resulting solution was stirred for 4 hours at room temperature, then partitioned between water and ethyl acetate. The organic fractions were washed with brine, dried over sodium sulfate, filtered and concentrated. The residue was purified by column chromatography, eluting with 10-30% ethyl acetate in petrol to afford N-benzoyl-amino-(2,4-difluoro-3-phenoxy-phenyl)-acetonitrile (971 mg) as a white solid. MS: [M+Na]⁺417.

### Step 2

Hydrogen chloride gas was bubbled through a solution of N-benzoyl-amino-(2,4-difluoro-3-phenoxy-phenyl)-acetonitrile (500 mg, 1.27 mmol) in ethanoldiethyl ether (1:1, 10 ml) at 0° C. The solution was stirred for 1 hour at 0° C., followed by 2 hours at room temperature, then stored at 4° C. for 72 hours. The solution was concentrated and triturated with diethyl ether.

The white solid was dissolved in anhydrous ethanol (5 ml) and ethylenediamine (2 ml) was added. The reaction was stirred for 3 hours at room temperature, then 1 hour at reflux, before being neutralized with sat. sodium hydrogen carbonate and extracted into DCM. Organic fractions were dried over sodium sulfate, filtered, concentrated and purified by column chromatography. Elution with 10% methanol in DCM generated [(2,4-difluoro-3-phenoxy-phenyl)-(4,5-dihydro-1H-imidazol-2-yl)-methyl]-carbamic acid benzyl ester (60 mg as a white solid. MS: [M+H₃O]⁺456.

### Step 3

To a solution of [(2,4-difluoro-3-phenoxy-phenyl)-(4,5-dihydro-1H-imidazol-2-yl)-methyl]-carbamic acid benzyl ester (50 mg, 0.11 mmol) in acetic acid (1 ml) at 0° C. was added HBr (2 ml of a 32% solution in AcOH) and the resulting mixture was stirred overnight. The suspension was filtered and the solid washed with copious volumes of diethyl ether, then dried to give the title compound (37 mg) as a yellow solid.

### Example 53

### 1-[1-(2,4-Difluoro-3-phenoxy-phenyl)-propylamino]-propan-2-ol.hydrochloride

A mixture of 1-(2,4-difluoro-3-phenoxy-phenyl)-propylamine (prepared as described in Example 3) (50 mg, 0.19 mmol) and 1-bromo-2-propanol (26 mg, 0.19 mmol) was heated under microwave irradiation at 120° C. for 8×15 min. The material was purified by preparative hplc to give the title compound (15 mg) as a 5:1 mixture of diastereomers.

### Example 54

### (S)-2-[(R)-1-(4-Chloro-2-fluoro-3-phenoxy-phenyl)-propylamino]-propionamide hydrochloride

### Step 1

(S)-2-[(R)-1-(4-chloro-2-fluoro-3-phenoxy-phenyl)-propylamino]-N-(2,4-dimethoxy-benzyl)-propionamide was prepared from the acid (Example 9 Step 2) according to the method described in Example 223 using 2-4-dimethoxybenzylamine. MS: [M+H]⁺501

### Step 2

A mixture of (S)-2-[(R)-1-(4-chloro-2-fluoro-3-phenoxy-phenyl)-propylamino]-N-(2,4-dimethoxy-benzyl)-propionamide (100 mg, 0.2 mmol), trifluoroacetic acid (1 ml), anisole (0.05 ml) and DCM (1 ml) was stirred at 70° C. overnight. The mixture was allowed to cool, extra DCM was added and the organic liquors were washed with saturated sodium bicarbonate solution and were concentrated. The residue was purified by column chromatography and was salted using 2N hydrochloric acid in ethyl acetate and dried in a vacuum oven. (S)-2-[(R)-1-(4-Chloro-2-fluoro-3-phenoxy-phenyl)-propylamino]-propionamide hydrochloride (16 mg) was produced.

### Example 55

### 4-[1-(2,4-Difluoro-3-phenoxy-phenyl)-propylamino]-tetrahydro-furan-3-ol.hydrochloride

A mixture of 1-(2,4-difluoro-3-phenoxy-phenyl)-propylamine prepared as described in Example 3 (50 mg, 0.19 mmol) and 3,4-epoxytetrahydrofuran (16 mg, 0.19 mmol) was heated under microwave irradiation at 140° C. for a total of 6 hours with further and 3,4-epoxytetrahydrofuran (16 mg, 0.19 mmol) added ever hour. The material was purified by preparative hplc to give the title compound (24 mg) as a 2:3 mixture of diastereomers. ¹H NMR (400 MHz, Me-d3-OD): 7.50-7.39 (1H, m), 7.39-7.24 (3H, m), 7.16-7.06 (1H, m), 6.95 (2H, d), 4.57-4.18 (2H, m), 4.13-3.96 (2H, m), 3.94-3.62 (1H, m), 3.62-3.53 (1H, m), 2.04 (2H, d), 0.95-0.83 (3H, m).

### Example 56

### 3-[1-(2,4-Difluoro-3-phenoxy-phenyl)-propylamino]-propan-1-ol.hydrochloride

[3-(tert-Butyl-dimethyl-silanyloxy)-propyl]-[1-(2,4-difluoro-3-phenoxy-phenyl)-propyl]-amine (prepared in an analogous fashion to Example 56 using 3-(tert-butyldimethylsiloxy)-propanal in step 1) (126 mg, 0.29 mmol) was dissolved in THF (3 ml) and tetrabutyl ammonium fluoride (0.58 ml of a 1M solution in THF, 0.58 mmol) was added. The reaction mixture was stirred for 1 hour at room temperature, concentrated and purified by preparative hplc to give the title compound (55 mg) as a solid.

### Examples 59 and 60

### C-(2,4-Difluoro-3-phenoxy-phenyl)-C-pyridin-3-yl-methylamine. dihydrochloride (Example 59A); C-(2,4-Difluoro-3-phenoxy-phenyl)-C-piperidin-3-yl-methylamine. dihydrochloride (anti-diastereomer) (Example 59B) and C-(2,4-Difluoro-3-phenoxy-phenyl)-C-piperidin-3-yl-methylamine.dihydrochloride (Syn-diastereomer) (Example 60)

### Step 1

3-Bromopyridine (590 mg, 3.7 mmol) in diethyl ether (5 ml) was added dropwise to a solution of n-butyl lithium (1.5 ml of a 2.5M solution in hexanes) in diethyl ether (15 ml) at −78° C. under an inert atmosphere. After stirring at this temperature for 30 mins, a cooled solution (−78° C.) of 2-methyl-propane-2-sulfinic acid 1-(2,4-difluoro-3-phenoxy-phenyl)-meth-(E)-ylideneamide (Prepared as described in Example 1) (500 mg, 1.5 mmol) in THF (8 ml) was added. The reaction was stirred at this temperature for a further 1.5 hours, then quenched with sat. ammonium chloride (3 ml) and allowed to warm to room temperature, before being partitioned between water and DCM. The organic fractions were dried over sodium sulfate, filtered and concentrated and the residue was purified by column chromatography, eluting with 70% ethyl acetate in petrol. The resulting white foam was redissolved in methanol (6 ml) and HCl (3 ml of a 4M solution in dioxanes, 12 mmol) was added and the reaction mixture stirred for 1 hour at room temperature. The resulting suspension was filtered and the solid washed with diethyl ether and dried to afford C-(2,4-Difluoro-3-phenoxy-phenyl)-C-pyridin-3-yl-methylamine. dihydrochloride (Example 59A) (374 mg) as an off-white solid. MS: [M+H]⁺313.

### Step 2

A suspension of platinum dioxide (60 mg, 0.052 mmol) and C-(2,4-difluoro-3-phenoxy-phenyl)-C-pyridin-3-yl-methylamine hydrochloride (200 mg, 0.52 mmol) in methanolethanol1-propanolDMF (1:1:1:1, 10 ml) was flushed with N₂ before being stirred under a hydrogen atmosphere for 6 hours. The mixture was filtered and the filtrate evaporated to dryness. The residue was purified by preparative hplc to afford -(2,4-Difluoro-3-phenoxy-phenyl)-C-piperidin-3-yl-methylamine. dihydrochloride (anti-diastereomer) (Example 59B) (7 mg) as a white solid. Further elution yielded C-(2,4-Difluoro-3-phenoxy-phenyl)-C-piperidin-3-yl-methylamine. dihydrochloride (Syn-diastereomer) (Example 60) (24 mg) also as a white solid.

### Examples 61 and 62

### C-(2,4-Difluoro-3-phenoxy-phenyl)-C-(tetrahydrofuran-3-yl)-methylamine.hydrochloride (anti-diastereomer) and C-(2,4-Difluoro-3-phenoxy-phenyl)—C-(tetrahydro-furan-3-yl)-methylamine.hydrochloride (Syn-diastereomer)

### Step 1

A solution of 1,6-difluorophenol (10.12 g, 78 mmol), tert-butyldimethylsilyl chloride (9.3 g, 62 mmol) and imidazole (6 g, 88 mmol) in DMF (50 ml) was stirred overnight under an inert atmosphere. The reaction mixture was partitioned between water and petrol and the combined organic fractions were washed with water, 10% potassium carbonate and brine, dried over sodium sulfate, filtered and evaporated. The residue was purified by column chromatography. Elution with petrol afforded 2-(tert-butyldimethylsilyloxy)-1,3-difluoro-benzene (13.74 g as a colourless oil). ¹H NMR (400 MHz, DMSO-d₆): 7.19-7.04 (2H, m), 7.04-6.92 (1H, m), 0.98 (9H, s), 0.17 (6H, s).

### Step 2

A solution of tetrahydrofuran-3-carboxaldehyde (2.45 g, 24.5 mmol), tert-butylsulfinamide (3.11 g (25.7 mmol) and titanium tetraethoxide (11.2 g, 50 mmol) in DCM (20 ml) was stirred overnight before brine (20 ml) was added. The suspension was filtered through celite and the filtrate extracted with DCM. The combined organic fractions were dried over sodium sulfate, filtered and concentrated and the residue purified by column chromatography. Elution with 30% ethyl acetate in petrol generated 2-methyl-propane-2-sulfinic acid 1-(tetrahydro-furan-3-yl)-meth-(E)-ylideneamide (2.8 g) as a pale yellow oil.

### Step 3

sec-butyl lithium (3.15 ml of a 1.3M solution in cyclohexane, 4.1 mmol) was added dropwise to a solution of 2-(tert-butyldimethylsilyloxy)-1,3-difluorobenzene (1.0 g, 4.1 mmol) in THF (10 ml) at −78° C. under an inert atmosphere. After 30 mins at this temperature, a solution of 2-methyl-propane-2-sulfinic acid 1-(tetrahydro-furan-3-yl)-meth-(E)-ylideneamide (693 mg, 3.4 mmol) in THF (5 ml). The reaction was stirred for 1 hour at −78° C., before being quenched with sat. ammonium chloride (10 ml) and allowed to warm to room temperature. The layers were separated and the aqueous portion was further extracted with DCM. The organic fractions were dried over sodium sulfate, filtered and evaporated to dryness. The residue was purified by column chromatography, eluting with 60% ethyl acetate in petrol gave 2-methyl-propane-2-sulfinic acid [[3-(tert-butyl-dimethyl-silanyloxy)-2,4-difluoro-phenyl]-(tetrahydro-furan-3-yl)-methyl]amide (715 mg) as a white foam. MS: [M+H]⁺448.

### Step 4

To a solution of 2-methyl-propane-2-sulfinic acid [[3-(tert-butyl-dimethyl-silanyloxy)-2,4-difluoro-phenyl]-(tetrahydro-furan-3-yl)-methyl]-amide (715 mg, 1.6 mmol) in acetonitrile (4.75 ml) and water (0.25 ml) was added 1,8-diazabicycloundec-7-ene (0.24 ml, 1.6 mmol) and the resulting solution was stirred for 1 hour. The reaction was partitioned between sat. ammonium chloride and DCM. The organic fractions were dried over sodium sulfate, filtered and concentrated and the residue purified by column chromatography. Elution with ethyl acetate gave 2-methyl-propane-2-sulfinic acid [(2,4-difluoro-3-hydroxy-phenyl)-(tetrahydro-furan-3-yl)-methyl]-amide (400 mg) as a white foam. MS: [M+H]⁺334.

### Step 5

2-Methyl-propane-2-sulfinic acid [(2,4-difluoro-3-hydroxy-phenyl)-(tetrahydro-furan-3-yl)-methyl]-amide (385 mg, 1.15 mmol) was coupled with phenyl boronic acid (352 mg, 2.9 mmol) using the method described in Key Intermediate 1, step 1. The residue was dissolved in methanol (3 ml) and HCl (3 ml of a 4M solution in dioxane) was added. After 1 hour, the solution was evaporated to dryness and the residue purified by preparative hplc to afford the anti diastereomer (Example 61) (30 mg) as a white foam. Further elution yielded the syn diastereomer (Example 62) (30 mg) as a white foam.

### Example 72

### 1-(2,4-Difluoro-3-phenoxy-phenyl)-2-pyridin-4-yl-ethylamine (Example 72A) and 1-(2,4-Difluoro-3-phenoxy-phenyl)-2-piperidin-4-yl-ethylamine. dihydrochloride (Example 72B)

### Step 1

A solution of 4-methylpyridine (280 mg, 2.9 mmol) in THF (4 ml) was cooled to 0° C. and lithium hexadimethylsilazide (2.9 ml of a 1M solution in THF, 2.9 mmol) was added under an inert atmosphere. The resulting solution was stirred 30 mins. further at this temperature and a solution of 2-methyl-propane-2-sulfinic acid 1-(2,4-difluoro-3-phenoxy-phenyl)-meth-(E)-ylideneamide (prepared as described in Example 1) (500 mg, 0.1.48 mmol) in THF (6 ml) was added dropwise. The reaction mixture was allowed to warm to room temperature and stirred for 1 hour before being quenched with sat. ammonium chloride. The layers were separated and the aqueous portion further extracted with DCM. The organic fractions were dried over sodium sulfate, filtered and concentrated. The residue was purified by column chromatography, eluting with 50-100% ethyl acetate in petrol afforded the product (342 mg) as a yellow gum. This was redissolved in methanol (3 ml) and HCl (3 ml of a 4M solution in dioxane) was added. After 1 hour the solvent was evaporated and the residue triturated with diethyl ether to give 1-(2,4-difluoro-3-phenoxy-phenyl)-2-pyridin-4-yl-ethylamine (Example 72A) as a pale yellow solid. MS: [M+H−NH₃]⁺310.

### Step 2

1-(2,4-difluoro-3-phenoxy-phenyl)-2-pyridin-4-yl-ethylamine (259 mg, 0.8 mmol) was reduced as described in Example 59, step 2 to generate difluoro-3-phenoxy-phenyl)-2-piperidin-4-yl-ethylamine. dihydrochloride (Example 72B) (121 mg) as a white solid.

### Example 73

### 5-[Amino-(2,4-difluoro-3-phenoxy-phenyl)-methyl]-1H-pyridin-2-one

### Step 1

THF (10 ml) and di-iso-butyl aluminium hydride (0.04 ml of a 1M solution in toluene, 0.04 mmol) were added to a mixture of magnesium (690 mg, 28.3 mmol) and lithium chloride (190 mg, 4.5 mmol) under an argon atmosphere. The resulting mixture was cooled to 0° C. and 5-bromo-2-chloropyridine (690 mg, 3.6 mmol) was added in one portion. After 30 mins, a solution of 2-methyl-propane-2-sulfinic acid 1-(2,4-difluoro-3-phenoxy-phenyl)-meth-(E)-ylideneamide (prepared as described in Example 1) (1.205 g, 3.6 mmol) in THF (6 ml) was added and the reaction was allowed to warm to room temperature and stirred for 1.5 hours. The mixture was cooled to 0° C. and quenched with sat. ammonium chloride, then extracted into DCM. The combined organic extracts were dried over sodium sulfate, filtered and evaporated to dryness. The residue was purified by column chromatography. Elution with 25-50% ethyl acetate in petrol yielded 2-methyl-propane-2-sulfinic acid [(6-chloro-pyridin-3-yl)-(2,4-difluoro-3-phenoxy-phenyl)-methyl]-amide (170 mg) as a colourless oil.

### Step 2

A solution of 2-methyl-propane-2-sulfinic acid [(6-chloro-pyridin-3-yl)-(2,4-difluoro-3-phenoxy-phenyl)-methyl]-amide (170 mg, 0.38 mmol) in 6N HCl (5 ml) was heated to reflux overnight, before being concentrated. The residue was purified by preparative hplc to afford 5-[amino-(2,4-difluoro-3-phenoxy-phenyl)methyl]-1H-pyridin-2-one (42 mg) as a white solid. MS: [M+H]⁺329.

### Step 3

A solution of 5-[amino-(2,4-difluoro-3-phenoxy-phenyl)-methyl]-1H-pyridin-2-one (30 mg, 0.09 mmol) in acetic acid (2 ml) was stirred for 16 hours under a 50 psi atmosphere of hydrogen. The resulting suspension was filtered and the filtrate concentrated, azeotroping with methanol. The residue was purified by preparative hplc to afford the syn diastereoisomer (10 mg) of the title compound as a colourless gum. Further elution yielded the anti diastereomer (16 mg) as a colourless gum.

### Examples 75 and 76

### 2-{[(2,4-Difluoro-3-phenoxy-phenyl)-piperidin-4-yl-methyl]-amino}-propan-1-ol.dihydrochloride (diastereoisomer 1) (Example 75)

### 2-{[(2,4-Difluoro-3-phenoxy-phenyl)-piperidin-4-yl-methyl]-amino}-propan-1-ol.dihydrochloride (diastereoisomer 2) (Example 76)

### Step 1

sec-butyl lithium (42.2 ml of a 1.3M solution in cyclohexane, 54.9 mmol) was added dropwise to a solution of 2-(tert-butyldimethylsilyloxy)-1,3-difluorobenzene (prepared as described in Example 61, step 1) (9.05 g, 37.2 mmol) in THF (100 ml) at −70° C. under an inert atmosphere. After 30 mins at this temperature, a solution of 4-{[(E)-2-methyl-propane-2-sulfinylimino]-methyl}-piperidine-1-carboxylic acid tert-butyl ester (prepared analogously to Example 61, step 2) (11.25 g, 35.4 mmol) in THF (50 ml) was added dropwise, maintaining a temperature below −60° C. The reaction was stirred for 1 hour further at this temperature, before tetrabutyl ammonium fluoride (39 ml of a 1M solution in THF, 39 mmol) was added. The reaction was allowed to warm to room temperature and stirred for 1 hour, then partitioned between diethyl ether and brine. The organic fractions were washed extensively with water, dried over sodium sulfate, filtered and evaporated to dryness. The aqueous fraction was further extracted with ethyl acetate and the organic fractions dried, filtered and concentrated. The two residues were combined to give 4-[(2,4-difluoro-3-hydroxy-phenyl)-(2-methyl-propane-2-sulfinylamino)-methyl]-piperidine-1-carboxylic acid tert-butyl ester (15 g) as a white foam, which was used without further purification.

### Step 2

4-[(2,4-Difluoro-3-hydroxy-phenyl)-(2-methyl-propane-2-sulfinylamino)-methyl]-piperidine-1-carboxylic acid tert-butyl ester (1.5 g, 3.35 mmol) was coupled with phenyl boronic acid (610 mg, 5.03 mmol) using the method described in Key Intermediate 1, step 1. The residue (1.7 g) was dissolved in diethyl ether (10 ml) and cooled to 0° C. HCl (0.84 ml of a 4M solution in dioxane, 3.35 mmol) was added. The reaction was stirred at 0° C. for 1 hour, then at room temperature for 48 hours. The resulting suspension was filtered and the filtrate concentrated and the residue purified by column chromatography. Elution with 0-15% methanol in DCM afforded 4-[amino-(2,4-difluoro-3-phenoxy-phenyl)-methyl]-piperidine-1-carboxylic acid tert-butyl ester (780 mg) as a pale brown gum. MS: [M+Na]⁺441.

### Step 3

4-[Amino-(2,4-difluoro-3-phenoxy-phenyl)-methyl]-piperidine-1-carboxylic acid tert-butyl ester was treated with hydroxyacetone and then with HCl as described in Example 56. The product was purified by column chromatography, eluting with 10% methanol in DCM to afford one diastereomer (20 mg) (Example 75) as an off-white solid. Further elution yielded the other diastereomer (20 mg) (Example 76) also as an off-white solid.

### Examples 79 and 80

### (S)-3-[(S)-1-(4-Chloro-2-fluoro-3-phenoxy-phenyl)-propylamino]-butyramide.hydrochloride (Example 79) and (R)-3-[(S)-1-(4-Chloro-2-fluoro-3-phenoxy-phenyl)-propylamino]-butyramide.hydrochloride (Example 80)

(S)-1-(4-Chloro-2-fluoro-3-phenoxy-phenyl)-propylamine (prepared in analogous manner to Key Intermediate 1, but using 6-chloro-2-fluoro-3-methyl phenol as starting material) (80 mg, 0.25 mmol) was reductively aminated with acetoacetamide using the method described in Example 56, step 1. The diastereomers were separated by column chromatography. Elution with 0-50% methanol in DCM afforded the (R,S) product (50 mg) as a white solid. Further elution yielded the (S,S) isomer (7 mg), also as a white solid.

### Example 87

### 2-[(R)-1-(2,4-Difluoro-3-phenoxy-phenyl)-2-pyridin-4-yl-ethylamino]-ethanol (Example 87A) and 2-[(R)-1-(2,4-Difluoro-3-phenoxy-phenyl)-2-piperidin-4-yl-ethylamino]-ethanol (Example 87B)

### Step 1

To a suspension of (S)-1-(2,4-difluoro-3-phenoxy-phenyl)-2-pyridin-4-yl-ethylamine (150 mg, 0.38 mmol) (prepared as described in Example 72, but using (R)-tert-butyl sulfinimide) in DCE (3 ml) was added triethylamine (0.1 ml, 7.6 mmol), 2-(tert-butyldimethylsilyloxy)-ethanal (0.07 ml, 0.38 mmol) and sodium triacetoxyborohydride (112 mg, 5.3 mmol) and the resulting mixture was stirred overnight at room temperature. The reaction was partitioned between 1M sodium hydroxide and DCM. The combined organic extracts were dried over sodium sulfate, filtered and concentrated. The crude residue was redissolved in THF (2 ml) and tetrabutyl ammonium fluoride (0.38 ml of a 1M solution in THF, 0.38 ml) was added. After stirring for 1.5 hours, the reaction mixture was partitioned between sat. ammonium chloride and DCM. The organic fractions were dried over sodium sulfate, filtered and concentrated and the residue purified by column chromatography. Elution with 5-10% methanol in DCM gave 2-[(S)-1-(2,4-difluoro-3-phenoxy-phenyl)-2-pyridin-4-yl-ethylamino]-ethanol (Example 87A) (140 mg) as a yellow oil. MS: [M+H]⁺371.

### Step 2

2-[(S)-1-(2,4-difluoro-3-phenoxy-phenyl)-2-pyridin-4-yl-ethylamino]-ethanol (240 mg, 0.65 mmol) was reduced as described in Example 59, step 2 but using methanol as the solvent to generate Example 87B) (20 mg) as a white solid.

### Example 88

### (S)-3-[(R)-1-(4-Chloro-2-fluoro-3-phenoxy-phenyl)-propylamino]-butyramide.hydrochloride

### Step 1

6-Chloro-2-fluoro-3-methylphenol (35 g, 0.218 mol), cesium fluoride (100 g, 0.654 mol) and acetonitrile (350 mL) were combined, stirring at room temperature under nitrogen. 2-(Trimethylsilyl)phenyl triflate (65 g, 0.218 mol) in acetonitrile (100 mL) was added over 20 minutes, followed by acetonitrile (250 mL). The resulting mixture was stirred at room temperature overnight. The reaction was quenched with 10% aqueous potassium hydroxide (350 mL) and extracted with petrol (7×700 mL). The combined organics were dried (magnesium sulfate) and concentrated in vacuo at 40° C. to give 1-chloro-3-fluoro-4-methyl-2-phenoxybenzene (44.5 g, 0.188 mol).

### Step 2

1-Chloro-3-fluoro-4-methyl-2-phenoxybenzene (44.5 g, 0.188 mol), N-bromosuccinimide (100.4 g, 0.564 mol), azobisisobutyronitrile (2.2 g, 0.013 mol) and carbon tetrachloride (445 mL) were stirred under nitrogen and heated to 80° C. overnight. Further N-bromosuccinimide (20 g, 0.112 mol) and azobisisobutyronitrile (2.2 g, 0.013 mol) were added. Heating was continued for a further 6 hrs, when the reaction was complete by ¹H NMR. Heating was removed and the reaction mixture was cooled to room temperature. Water (440 mL) was added and the phases were separated. The aqueous was extracted with dichloromethane (2×220 mL) and the combined organics were dried (magnesium sulfate) and concentrated in vacuo at 40° C. to give 1-chloro-4-dibromomethyl-3-fluoro-2-phenoxybenzene (98.3 g). The material was used directly without purification.

### Step 3

1-Chloro-4-dibromomethyl-3-fluoro-2-phenoxybenzene (98.3 g), isopropanol (740 mL), silver nitrate (64 g, 0.376 mol) and water (150 mL) were combined. The resulting mixture was stirred for 2 hrs and then filtered. The filtrate was concentrated in vacuo at 40° C. and water (375 mL) was added to the residue. The mixture was extracted with dichloromethane (2×375 mL) and the combined organics were dried (magnesium sulfate) and concentrated in vacuo at 40° C. The residue was chromatographed on a silica pad, eluting with a gradient of 5-10% ethyl acetatepetrol to give 4-chloro-2-fluoro-3-phenoxybenzaldehyde (31 g, 0.123 mol).

### Step 4

4-Chloro-2-fluoro-3-phenoxybenzaldehyde (37.8 g), (R)-(+)-2-methyl-2-propanesulfinamide (19.1 g, 0.158 mol), titanium(IV) ethoxide (68.8 g, 0.301 mol) and dichloromethane (565 mL) were combined. The resulting mixture was stirred overnight under nitrogen. The solution was diluted with dichloromethane (565 mL) and solid sodium sulfate decahydrate (380 g) was added with vigorous stirring for 1 hr. The slurry was filtered and the filtrate was concentrated in vacuo at 40° C. The residue was chromatographed on a silica pad, eluting with a gradient of 0-20% ethyl acetatepetrol to give (R)-2-methylpropane-2-sulfinic acid 1-(4-chloro-2-fluoro-3-phenoxyphenyl)meth-(E)-ylideneamide (26.8 g, 0.076 mol).

### Step 5

A solution of ethylmagnesium bromide (50 mL, 0.15 mol) was added over 35 minutes to a solution of (R)-2-methylpropane-2-sulfinic acid 1-(4-chloro-2-fluoro-3-phenoxyphenyl)meth-(E)-ylideneamide (26.5 g) in tetrahydrofuran (530 mL) at −70° C. After 3 hrs stirring at −70° C., the mixture was quenched with saturated ammonium chloride (270 mL). Water (270 mL) was added and the phases were separated. The aqueous was extracted with ethyl acetate (2×270 mL) and the combined organics were washed with saturated brine (270 mL), dried (magnesium sulfate) and concentrated in vacuo at 40° C. The residue was chromatographed on a silica pad, eluting with a gradient of 20-60% ethyl acetatepetrol to give (R)-2-methylpropane-2-sulfinic acid [(R)-1-(4-chloro-2-fluoro-3-phenoxyphenyl)-propyl]amide (11.9 g, 0.031 mol).

### Step 6

4M Hydrogen chloride in dioxane (24 mL) was added to a solution of (R)-2-methylpropane-2-sulfinic acid [(R)-1-(4-chloro-2-fluoro-3-phenoxyphenyl)propyl]amide (11.9 g, 0.031 mol) in methanol (120 mL). After stirring for 1 hr, the solution was concentrated in vacuo at 40° C. The residue was slurried in 3:1 petrolether (120 mL), filtered and dried in vacuo at 40° C. to give (R)-1-(4-chloro-2-fluoro-3-phenoxyphenyl)propylamine hydrochloride (9.3 g, 0.029 mol).

### Step 7

Triethylamine (0.04 ml, 0.25 mmol) was added to a mixture of (R)-1-(4-chloro-2-fluoro-3-phenoxy-phenyl)propylamine hydrochloride (80 mg, 0.25 mmol) and acetoacetamide (26 mg, 0.25 mmol) in DCE (3 ml), followed by glacial acetic acid (0.04 ml, 0.5 mmol) and sodium triacetoxyborohydride (164 mg, 0.5 mmol). The resulting mixture was stirred at room temperature for 24 hours, poured into saturated sodium hydrogen carbonate and extracted into DCM. The organic fraction was dried over sodium sulfate, filtered and concentrated. The diastereomers were separated by column chromatography. Elution with 0-10% methanol in DCM afforded the (R,R) isomer which was subsequently converted to the hydrochloride salt (35 mg). Further elution provided the (S,R) isomer which was subsequently converted to the title compound hydrochloride salt (3 mg).

### Example 91

### N-Cyanomethyl-3-[(S)-1-(2,4-difluoro-3-phenoxy-phenyl)-propylamino]-propionamide.hydrochloride

### Step 1

(R)-1-(2,4-Difluoro-3-phenoxy-phenyl)-propylamine was reacted with ethyl acrylate in a microwave oven to give 3-[(R)-1-(2,4-difluoro-3-phenoxy-phenyl)-propylamino]-propionic acid ethyl ester.

### Step 2

Lithium hydroxide (152 mg, 3.7 mmol) was added to a solution of 3-[(R)-1-(2,4-difluoro-3-phenoxy-phenyl)-propylamino]-propionic acid ethyl ester (658 mg, 1.8 mmol) in THF:methanol:water (2:1:1, 5 ml) and the reaction stirred at room temperature for 1 hour. The mixture was adjusted to pH 7 using 2M HCl then evaporated to dryness. The residue was dissolved in DMSO and purified by preparative hplc to give of 3-[(R)-1-(2,4-difluoro-3-phenoxy-phenyl)-propylamino]-propionic acid (180 mg) as an off-white solid. MS: [M−H]⁻ 334.

### Step 3

A solution of 3-[(R)-1-(2,4-difluoro-3-phenoxy-phenyl)-propylamino]-propionic acid (67 mg, 0.2 mml), 1-ethyl-3-(3-dimethylaminopropyl)carbodiimide (42 mg, 0.22 mmol), 1-hydroxybenzotriazole (30 mg, 0.22 mmol) and aminoacetonitrile (11.3 mg, 0.2 mmol) in DMSO (1 ml) was stirred at room temperature overnight. The solution was purified by preparative hplc to afford the title compound (10 mg) as a solid.

### Example 92

### 3-[(S)-1-(2,4-Difluoro-3-phenoxy-phenyl)-propylamino]-N-(2-hydroxy-ethyl)-propionamide.hydrochloride

### Step 1

3-[(R)-1-(2,4-Difluoro-3-phenoxy-phenyl)-propylamino]-propionic acid (prepared as described in Example 91) (67 mg, 0.2 mml) was treated with 2-(tert-butyldimethylsilanyloxy)-ethylamine as described in Example 91, step 3 to afford N-[2-(tert-butyl-dimethyl-silanyloxy)-ethyl]-3-[(R)-1-(2,4-difluoro-3-phenoxy-phenyl)-propylamino]-propionamide as a solid. MS: [M+H]⁺493.

### Step 2

A solution of N-[2-(tert-butyl-dimethyl-silanyloxy)-ethyl]-3-[(R)-1-(2,4-difluoro-3-phenoxy-phenyl)-propylamino]-propionamide (103 mg, 0.21 mmol) and tetrabutyl ammonium fluoride (0.42 ml of a 1M solution in THF, 0.42 mmol) in THF (1 ml) was stirred at room temperature for 2 hours, then concentrated. The residue was purified by preparative hplc to generate the title compound (28 mg) as a solid.

### Example 95

### 2-[1-(2,4-Difluoro-3-phenoxy-phenyl)-2-pyridin-4-yl-ethylamino]-propan-1-ol (Example 95A) and (R)-2-[(S)-1-(2,4-Difluoro-3-phenoxy-phenyl)-2-piperidin-4-yl-ethylamino]-propan-1-ol.dihydrochloride (Example 95B)

### Step 1

(S)-1-(2,4-difluoro-3-phenoxy-phenyl)-2-pyridin-4-yl-ethylamine (prepared as described in example 72 using (S)-tert-butylsulfinimide) (250 mg, 0.63 mmol) was treated with hydroxyacetone as described in Example 56, step 1 to generate 2-[(S)-1-(2,4-difluoro-3-phenoxy-phenyl)-2-pyridin-4-yl-ethylamino]-propan-1-ol (Example 95A) (200 mg) as a 2:1 mixture of diastereomers. MS: [M+H]⁺385.

### Step 2

2-[(S)-1-(2,4-Difluoro-3-phenoxy-phenyl)-2-pyridin-4-yl-ethylamino]-propan-1-ol (170 mg, 0.44 mmol) was reduced as described in Example 59, step 2 to give a mixture of diastereoisomers of (R)-2-[(S)-1-(2,4-difluoro-3-phenoxy-phenyl)-2-piperidin-4-yl-ethylamino]-propan-1-ol. dihydrochloride. The diastereomers were separated by preparative hplc to give the (S,S) diastereomer (36 mg) as a white solid. Further elution yielded the (S,R) diastereomer (29 mg also as a white solid.

### Example 97

### 3-[(R)-1-(4-Chloro-2-fluoro-3-phenoxy-phenyl)-propylamino]-propionamide.hydrochloride

A mixture of (S)-1-(2-chloro-4-fluoro-3-phenoxy-phenyl)-propylamine (prepared analogously to Key Intermediate 1) (50 mg, 0.16 mmol), triethylamine (0.02 ml, 0.16 mmol) and 3-bromopropionamide (24 mg, 0.16 mmol) was heated under microwave irradiation to 120° C. for 2×30 mins. The resulting mixture was purified by preparative hplc to afford the title compound (7 mg) as a solid.

### Example 100

### 2-[(S)-1-(2,4-Difluoro-3-phenoxy-phenyl)-propylamino]-ethanol.trifluoroacetate

### Step 1

Key Intermediate 1 (200 mg, 0.67 mmol) was treated with (tert-butyldimethylsilyloxy)-acetaldehyde (0.14 ml, 0.67 mmol) using the method described in Example 3, step 2 to generate butyl-dimethyl-silanyloxy)-ethyl]-[(S)-1-(2,4-difluoro-3-phenoxy-phenyl)-propyl]-amine (281 mg) as a solid. MS: [M+H]⁺247.

Step 2

[2-(tert-Butyl-dimethyl-silanyloxy)-ethyl]-[(S)-1-(2,4-difluoro-3-phenoxy-phenyl)-propyl]-amine (170 mg, 0.55 mmol) was treated with tetrabutyl ammonium fluoride as described in Example 56 to afford the title compound (35 mg) as a white solid.

### Example 102

### Allyl-[(S)-1-(2,4-difluoro-3-phenoxy-phenyl)-propyl]-amine.hydrochloride

A mixture of allyl bromide (0.087 ml, 1.0 mmol) and Key Intermediate 1 (300 mg, 1.0 mmol) was stirred overnight and the resulting solid purified by preparative hplc to afford the title compound (89 mg) as a white solid.

### Example 103

### 2-[(S)-1-(2,4-Difluoro-3-phenoxy-phenyl)-propylamino]-ethanethiol.hydrochloride

### Step 1

Mercaptoacetic acid (0.38 ml, 5.43 mmol) was added to a solution of chlorotriphenylmethane (1.54 ml, 5.97 mmol) and triethylamine (0.83 ml, 5.97 mmol) in toluene (15 ml). The resulting solution was stirred at room temperature overnight before being concentrated. The residue was partitioned between water and chloroform. The organic fractions were dried over sodium sulfate, filtered and concentrated to give trityl sulfanylacetic acid (2.19 g) which was used without further purification.

### Step 2

1-Ethyl-3-(3-dimethylaminopropyl)carbodiimide (288 mg, 1.5 mmol) was added to a solution of Key Intermediate 1 (300 mg, 1.0 mmol), trityl sulfanylacetic acid (502 mg, 1.5 mmol), 1-hydroxy-7-azabenzotriazole (204 mg, 1.5 mmol) and diisopropylethylamine (0.87 ml, 5.0 mmol) in DMF (8 ml). The reaction mixture was stirred at room temperature for 48 hours, then partitioned between water and ethyl acetate. The organic fractions were washed with 5% citric acid and with sat. sodium hydrogen carbonate, dried over sodium sulfate, filtered and concentrated. The residue was triturated with DCMdiethyl ether (1:1) to afford N-[1-(2,4-difluoro-3-phenoxy-phenyl)-propyl]-2-tritylsulfanyl-acetamide (345 mg) as a white powder. MS: [M−H]⁻578.

### Step 3

Borane (0.93 ml of a 1M solution in THF, 0.93 mmol) was added dropwise to a solution of N-[1-(2,4-difluoro-3-phenoxy-phenyl)-propyl]-2-tritylsulfanyl-acetamide (180 mg, 0.31 mmol) in THF (2 ml). The mixture was heated to 60° C. overnight, then cooled to 0° C. before being quenched with methanol (1 ml) and concentrated. The residue was taken up in DCM (3 ml) and trifluoroacetic acid (0.31 ml of a 1M solution in THF, 0.31 mmol) was added dropwise, followed by triethylsilane (0.055 ml, 0.34 mmol). The resulting mixture was stirred for 1 hour at room temperature, before sat. sodium hydrogen carbonate (2 ml) was added. After 30 mins, the layers were separated and the aqueous layer was further extracted with DCM. The combined organic fractions were washed with brine, dried over sodium sulfate, filtered and concentrated and the residue purified by preparative hplc to yield the title compound (8 mg) as a white solid.

### Example 104

### 2-{1-[1-(2,4-Difluoro-3-phenoxy-phenyl)-propylamino]-ethyl}-cyclohexanone.hydrochloride

### Step 1

1-(2,4-difluoro-3-phenoxy-phenyl)-propylamine prepared as described in Example 3 (186 mg, 0.7 mmol) was added to 2-acetylcyclohexanone (57 mg, 0.27 mmol) in DCE (3 ml), followed by glacial acetic acid (0.056 ml, 1.4 mmol) and sodium triacetoxyborohydride (212 mg, 1.4 mmol). The resulting mixture was stirred at room temperature overnight, then poured into sat. sodium hydrogen carbonate and extracted into ethyl acetate. The residue was purified by preparative hplc to afford of 2-{1-[1-(2,4-difluoro-3-phenoxy-phenyl)-propylamino]-ethyl}-cyclohexanol (69 mg). MS: [M+H]⁺362.

### Step 2

Dess-Martin periodinane (1,1,1-Triacetoxy-1,1-dihydro-1,2-benziodoxol-3(1H)-one) (90 mg, 0.23 mmol) was added to a solution of 2-{1-[1-(2,4-difluoro-3-phenoxy-phenyl)-propylamino]-ethyl}-cyclohexanol (69 mg, 0.19 mmol) in DCM (3 ml). The mixture was stirred at room temperature for 2 hours, treated with further acetic acid 1,1-diacetoxy-3-oxo-1L5-ioda-2-oxa-indan-1-yl ester (90 mg, 0.23 mmol) and stirred at room temperature for 48 hours. The reaction was partitioned between DCM and sat. sodium thiosulphate, organic fraction washed with sat. sodium hydrogen carbonate, brine and dried over sodium sulphate. The residue was purified by preparative hplc to yield the title compound (7 mg).

### Example 105

### 1-(2-Fluoro-3-phenoxy-4-vinyl-phenyl)-2-pyridin-4-yl-ethylamine (Example 105A) and 1-(4-Ethyl-2-fluoro-3-phenoxy-phenyl)-2-piperidin-4-yl-ethylamine.dihydrochloride (Example 105B)

### Step 1

2-Methyl-propane-2-sulfinic acid 1-(4-chloro-2-fluoro-3-phenoxy-phenyl)-methylideneamide (prepared in an analogous fashion to Key Intermediate 1, but using 6-chloro-2-fluoro-3-methyl phenol as starting material) (1.81 g) was treated with 4-methylpyridine as described in Example 72, step 1 to give 2-methyl-propane-2-sulfinic acid [1-(4-chloro-2-fluoro-3-phenoxy-phenyl)-2-pyridin-4-yl-ethyl]-amide (1.085 g) as a solid. MS: [M+H]⁺447.

### Step 2

A solution of 2-methyl-propane-2-sulfinic acid [1-(4-chloro-2-fluoro-3-phenoxy-phenyl)-2-pyridin-4-yl-ethyl]-amide (100 mg, 0.22 mmol), potassium vinyltrifluoroborate (30 mg, 0.22 mmol) and potassium phosphate (142 mg, 0.66 mmol) in dioxane (1.5 ml) and water (0.5 ml) was degassed by bubbling through nitrogen for 10 mins.

Tris(dibenzylideneacetone)dipalladium (0) (10 mg, 0.01 mmol) was added, followed by 2-dicyclohexylphosphino-2′,6′-dimethoxybiphenyl (9 mg, 0.02 mmol) and the resulting mixture was heated for 1 hour at 120° C. under microwave irradiation. The mixture was partitioned between water and ethyl acetate and the organic fractions were washed with brine, dried over magnesium sulfate, filtered and evaporated. The residue was purified by column chromatography. Elution with 0-100% ethyl acetate in hexane, followed by 0-10% methanol in ethyl acetate afforded 2-methyl-propane-2-sulfinic acid [1-(2-fluoro-3-phenoxy-4-vinyl-phenyl)-2-pyridin-4-yl-ethyl]-amide (60 mg) as a solid. MS: [M+H]⁺439.

### Step 3

2-Methyl-propane-2-sulfinic acid [1-(2-fluoro-3-phenoxy-4-vinyl-phenyl)-2-pyridin-4-yl-ethyl]-amide (60 mg, 0.14 mmol) was treated with HCl as described in Key Intermediate 1, step 6 to give 1-(2-fluoro-3-phenoxy-4-vinyl-phenyl)-2-pyridin-4-yl-ethylamine (Example 105A) (45 mg) as a solid.

### Step 4

1-(2-Fluoro-3-phenoxy-4-vinyl-phenyl)-2-pyridin-4-yl-ethylamine (45 mg) was reduced as described in Example 59 to afford Example 105B (20 mg) as an off-white solid.

### Example 106

### (R)—N-{4-[3-(1-Amino-propyl)-6-chloro-2-fluoro-phenoxy]-2-methyl-phenyl}-acetamide

### Step 1

To A solution of (S)-2-methyl-propane-2-sulfinic acid {(R)-1-[4-chloro-2-fluoro-3-(4-amino-3-methyl-phenoxy)-phenyl]-propyl}-amide as described in Example 112 steps 1-2 (50 mg, 0.12 mmol) in pyridine (1 ml) added a total of acetyl chloride (0.025 ml, 0.3 mmol) at 0° C. over 2 hours. The mixture was concentrated and the residue partitioned between ethyl acetate and water. The organic fraction was dried over sodium sulphate, filtered and concentrated to yield a crude intermediate product (46 mg) MS: [M+H]⁺442.

### Step 2

The crude intermediate product was dissolved in a 4M solution of HCl in ethyl acetate (2 ml) and stirred overnight. The mixture was concentrated and triturated with ethyl acetatediethyl ether [1:1] resulting suspension filtered to give the title compound (27 mg).

### Example 107

### (R)—N-(2-Amino-ethyl)-3-(4-chloro-2-fluoro-3-phenoxy-benzylamino)-butyramide.dihydrochloride

### Step 1

Triethylamine (0.28 ml, 1.99 mmol) was added to a mixture of 4-chloro-2-fluoro-3-phenoxybenzaldehyde prepared in an analogous manner to key intermediate 1 (500 mg, 1.99 mmol) and (R)-3-amino-butyric acid ethyl ester hydrochloride (334 mg, 1.99 mmol) in DCE (10 ml), followed by glacial acetic acid (0.23 ml, 3.98 mmol) and sodium triacetoxyborohydride (1.27 g, 5.97 mmol). The resulting mixture was stirred at room temperature for 24 hours, then poured into sodium hydrogen carbonate and extracted with DCM. The organic fraction was washed with brine, dried over sodium sulphate, filtered and concentrated. Residue purified by column chromatography to give (R)-3-(4-chloro-2-fluoro-3-phenoxy-benzylamino)-butyric acid ethyl ester (270 mg) MS: [M+H]⁺366.

### Step 2

(R)-3-(4-Chloro-2-fluoro-3-phenoxy-benzylamino)-butyric acid ethyl ester (120 mg, 0.32 mmol) in THF:MeOH:H₂O (6 ml) was treated with lithium hydroxide monohydrate (1.2 equivs) and stirred at room temperature for 2 hours to give (R)-3-(4-chloro-2-fluoro-3-phenoxy-benzylamino)-butyric acid then concentrated. Used without further purification.

### Step 3

To (R)-3-(4-chloro-2-fluoro-3-phenoxy-benzylamino)-butyric acid from previous step in DMF (6 ml) and diisopropylethylamine (0.33 ml, 2.24 mmol) and tert-butyl N-(2-aminoethyl)carbamate (105 mg, 0.64 mmol). Reaction cooled to 0° C. 2-(1H-7-azabenzotriazol-1-yl)-1,1,3,3-tetramethyluronium hexafluorophosphate (186 mg, 0.48 mmol) was added. The reaction mixture was stirred for 1 hour at 0° C. poured into water and extracted with DCM twice. The organic fractions were combined washed with brine, dried over sodium sulphate, filtered and concentrated. Residue purified by preparative hplc, product was treated with a 4M solution of HCl in ethyl acetate (3 ml) and stirred RT overnight. The mixture was concentrated to yield the title compound (40 mg).

### Example 108

### N-{3-[3-(1-Amino-2-pyridin-4-yl-ethyl)-2,6-difluoro-phenoxy]-phenyl}-methane-sulfonamide (Example 108A) and N-{3-[3-(1-Amino-2-piperidin-4-yl-ethyl)-2,6-difluoro-phenoxy]-phenyl}-methane-sulfonamide.dihydrochloride (Example 1088)

### Step 1

Key Intermediate 2 (3.26 g, 8.7 mmol) was treated with 4-methylpyridine as described in Example 72, step 1 to generate (S)-2-methyl-propane-2-sulfinic acid {1-[3-(tert-butyl-dimethyl-silanyloxy)-2,4-difluoro-phenyl]-2-pyridin-4-yl-ethyl}-amide, which was used without further purification.

### Step 2

(S)-2-Methyl-propane-2-sulfinic acid {1-[3-(tert-butyl-dimethyl-silanyloxy)-2,4-difluoro-phenyl]-2-pyridin-4-yl-ethyl}-amide was treated with tetrabutyl ammonium fluoride as described in Example 56 to give (S)-2-methyl-propane-2-sulfinic acid [1-(2,4-difluoro-3-hydroxy-phenyl)-2-pyridin-4-yl-ethyl]-amide (480 mg) as a pale yellow solid. MS: [M+H]⁺355.

### Step 3

(S)-2-Methyl-propane-2-sulfinic acid [1-(2,4-difluoro-3-hydroxy-phenyl)-2-pyridin-4-yl-ethyl]-amide (308 mg, 0.87 mmol) was coupled with 3-(methanesulfonylamino)-phenyl boronic acid using the method described in Key Intermediate 1, step 1 to afford (S)-2-methyl-propane-2-sulfinic acid {1-[2,4-difluoro-3-(3-methylsulfonylamino-phenoxy)-phenyl]-2-pyridin-4-yl-ethyl}-amide (293 mg) as a brown gum. MS: [M+H]⁺524.

### Step 4

(S)-2-Methyl-propane-2-sulfinic acid {1-[2,4-difluoro-3-(3-methylsulfonylamino-phenoxy)-phenyl]-2-pyridin-4-yl-ethyl}-amide (290 mg) was treated with HCl as described in Key Intermediate 1, step 6 to give N-{3-[3-(1-amino-2-pyridin-4-yl-ethyl)-2,6-difluoro-phenoxy]-phenyl}-methane-sulfonamide (Example 108A) (270 mg) as an impure white powder. MS: [M−H]⁻ 418.

### Step 5

1-[2,4-difluoro-3-(3-methylsulfonylamino-phenoxy)-phenyl]-2-pyridin-4-yl-ethyl-amine (270 mg) was reduced as described in Example 59 to yield N-{3-[3-(1-amino-2-piperidin-4-yl-ethyl)-2,6-difluoro-phenoxy]-phenyl}-methane-sulfonamide. dihydrochloride (Example 108B) (79 mg) as an off-white solid.

### Example 110

### (2,4-Difluoro-3-phenoxy-benzyl)-pyridin-4-yl-amine.hydrochloride

### Step 1

To a stirred solution of 2,4-difluoro-3-methoxy-benzonitrile (2 g, 11.8 mmol) in DCM (59.1 mL) at −78° C. was added boron tribromide in DCM (35.5 mL, 35.5 mmol) slowly. The mixture was allowed to warm to room temperature and was stirred overnight. The mixture was cooled to 0° C. and additional boron tribromide in DCM (23.7 mL, 23.7 mmol) was added, the mixture was warmed to room temperature and stirred for 24 hours. The mixture was poured onto ˜200 mL of water and extracted into DCM (×3), dried (magnesium sulfate), filtered and concentrated to give 1.70 g of crude material. Trituration with DCM gave 1.11 g of 2,4-difluoro-3-hydroxy-benzonitrile as an off white powder. MS: [M−H]⁻ 154.

### Step 2

Difluoro-3-hydroxy-benzonitrile (0.287 g, 1.85 mmol) was treated with phenylboronic acid (0.677 g, 5.55 mmol) using the method described in Key Intermediate 1, step 1 to give 2,4-difluoro-3-phenoxy-benzonitrile, 281 mg.

### Step 3

To a stirred solution of 2,4-difluoro-3-phenoxy-benzonitrile (0.281 g, 1.22 mmol) in THF (3.04 mL) at 0° C. was added borane in THF (1M solution, 3.65 mL, 3.65 mmol) dropwise. The mixture was stirred at room temperature for 3 hours before it was quenched at 0° C. by the addition of excess MeOH (˜3 mL). The mixture was stirred at room temperature for 1 hour before THF was removed under vacuum and it was partitioned between water and EtOAc. The phases were separated and the aqueous layer was extracted into EtOAc (×3), combined organic extracts were dried (magnesium sulfate), filtered and concentrated. The residue was taken into DCM and 1.25M HCl in MeOH was added giving a white precipitate which was concentrated and triturated with Et2O giving 194 mg of 2,4-difluoro-3-phenoxy-benzylamine hydrochloride as a white solid. MS: [M−NH2]⁺ 219.

### Step 4

To a stirred suspension of 2,4-difluoro-3-phenoxy-benzylamine hydrochloride (0.095 g, 0.403 mmol) and 4-fluoropyridine hydrochloride (0.0538 g, 0.403 mmol) in MeCN (1.01 mL) at room temperature was added N,N-diisopropylethylamine (0.218 mL, 1.25 mmol). The solution was heated at 90° C. overnight, water and EtOAc were added, the phases were separated and the aqueous layer was extracted into EtOAc (×2). The combined organic extracts were dried (magnesium sulfate), filtered and concentrated to give 117 mg of crude material. Preparative HPLC gave the desired product as a free base. Formation of the HCl salt in Et2O gave 8.9 mg of (2,4-difluoro-3-phenoxy-benzyl)-pyridin-4-yl-amine hydrochloride as a white solid.

### Example 111

### {3-[3-((S)-1-Amino-propyl)-6-chloro-2-fluoro-phenoxy]-phenyl}-methanol.hydrochloride

### Step 1

The enantiomer of Key Intermediate 3, (S)-2-methyl-propane-2-sulfinic acid [(S)-1-(4-chloro-2-fluoro-3-hydroxy-phenyl)-propyl]-amide (300 mg, 0.98 mmol) was coupled with 3-formylphenyl boronic acid as described in Key Intermediate 1, step 1 to generate of (S)-2-methyl-propane-2-sulfinic acid {(S)-1-[4-chloro-2-fluoro-3-(3-formyl-phenoxy)-phenyl]-propyl}-amide (276 mg) as a colourless oil. MS: [M+H]⁺412.

### Step 2

To a solution of (S)-2-methyl-propane-2-sulfinic acid {(S)-1-[4-chloro-2-fluoro-3-(3-formyl-phenoxy)-phenyl]-propyl}-amide (276 mg, 0.67 mmol) in methanol (6 ml) at 0° C. was added sodium borohydride (51 mg, 1.34 mmol) and the resulting solution was stirred for 1 hour at this temperature. The mixture was concentrated and the residue partitioned between sat. ammonium chloride and DCM. The organic fractions were dried over sodium sulfate, filtered, concentrated and subjected to column chromatography. Elution with 50-70% ethyl acetate in petrol yielded (S)-2-methyl-propane-2-sulfinic acid {(S)-1-[4-chloro-2-fluoro-3-(3-hydroxymethyl-phenoxy)-phenyl]-propyl}-amide (252 mg) as a colourless gum. MS: [M+H]⁺414.

### Step 3

(S)-2-Methyl-propane-2-sulfinic acid {(S)-1-[4-chloro-2-fluoro-3-(3-hydroxymethyl-phenoxy)-phenyl]-propyl}-amide (200 mg) was treated with HCl as described in Key Intermediate 1, step 6 to afford the title compound (143 mg) as a white solid.

### Example 112

### 4-[3-((R)-1-Amino-propyl)-6-chloro-2-fluoro-phenoxy]-2-methyl-phenylamine.dihydrochloride

### Step 1

A solution of Key Intermediate 3 (300 mg, 0.98 mmol), 5-fluoro-2-nitrotoluene (0.14 ml, 1.17 mmol) and cesium carbonate (640 mg, 1.95 mmol) in DMSO (2 ml) was heated to 110° C. for 4 hours. The mixture was partitioned between brine and diethyl ether and the organic fraction dried over sodium sulfate, filtered and concentrated. The residue was purified by column chromatography, eluting with 30-50% ethyl acetate in petrol to give (R)-2-methyl-propane-2-sulfinic acid {(R)-1-[4-chloro-2-fluoro-3-(3-methyl-4-nitro-phenoxy)-phenyl]-propyl}-amide (286 mg as a yellow oil. MS: [M+H]⁺443.

### Step 2

A suspension of (R)-2-methyl-propane-2-sulfinic acid {(R)-1-[4-chloro-2-fluoro-3-(3-methyl-4-nitro-phenoxy)-phenyl]-propyl}-amide (230 mg, 0.52 mmol) and PdC (100 mg) in methanolethyl acetate (1:1, 5 ml) was stirred overnight under a hydrogen atmosphere. The reaction was filtered and the filtrate concentrated. The residue was purified by column chromatography, eluting with 2% methanol in DCM to afford (R)-2-methyl-propane-2-sulfinic acid {(R)-1-[4-chloro-2-fluoro-3-(4-amino-3-methyl-phenoxy)-phenyl]-propyl}-amide (230 mg) as a pale yellow oil. MS: [M+H]⁺413 mg.

### Step 3

(R)-2-Methyl-propane-2-sulfinic acid {(R)-1-[4-chloro-2-fluoro-3-(4-amino-3-methyl-phenoxy)-phenyl]-propyl}-amide (230 mg) was treated with HCl as described in Key Intermediate 1, step 6 to generate the title compound (84 mg) as a white solid.

### Example 113

### [1-(2,4-Difluoro-3-phenoxy-phenyl)-propyl]-pyridin-4-yl-amine.hydrochloride

A solution of 1-(2,4-difluoro-3-phenoxy-phenyl)-propylamine (prepared as Key Intermediate 1, using racemic sulfinimide) (100 mg, 0.3 mmol) and 4-chloropyridine hydrochloride (50 mg, 0.3 mmol) in NMP (1 ml) was heated to 140° C. for 1 hour under microwave irradiation. The reaction was purified by preparative hplc to afford the title compound (9 mg) as an off-white solid.

### Example 131

### (S)-3-[(R)-1-(4-Chloro-2-fluoro-3-phenoxy-phenyl)-propylamino]-N-methyl-butyramide.hydrochloride (Example 131A); and (R)-3-[(R)-1-(4-Chloro-2-fluoro-3-phenoxy-phenyl)-propylamino]-N-methyl-butyramide. hydrochloride (Example 131B)

### Step 1

A solution of (R)-1-(4-chloro-2-fluoro-3-phenoxy-phenyl)-propylamine

(prepared in an analogous fashion to Key Intermediate 1) (350 mg, 1.25 mmol) and methyl crotonate (0.13 ml, 1.25 mmol) in methanol (3 ml) was heated to 80° C. for 2×2 hours under microwave irradiation. Methyl crotonate (0.13 ml, 1.25 mmol) was added and the reaction further heated to 130° C. for 3 hours under microwave irradiation, before being concentrated. The residue was purified by column chromatography, eluting with 30-40% ethyl acetate in petrol to give 3-[(R)-1-(4-chloro-2-fluoro-3-phenoxy-phenyl)-propylamino]-butyric acid methyl ester (245 mg) as a mixture of diastereomers. MS: [M+H]⁺380.

### Step 1 Alternative Procedure

A solution of (R)-1-(4-chloro-2-fluoro-3-phenoxy-phenyl)-propylamine (prepared in an analogous fashion to Key Intermediate 1) (1 g, 3.16 mmol) in methyl crotonate (9 ml, excess) was heated to 170° C. for 6+2 hours under microwave irradiation, before being concentrated. The residue was purified by column chromatography, eluting with 0-45% ethyl acetate in petrol to give of 3-[(R)-1-(4-chloro-2-fluoro-3-phenoxy-phenyl)-propylamino]-butyric acid methyl ester (743 mg) as a mixture of diastereomers. MS: [M+H]⁺380.

### Step 2

A solution of 3-[(R)-1-(4-chloro-2-fluoro-3-phenoxy-phenyl)-propylamino]-butyric acid methyl ester (235 mg, 0.62 mmol) and lithium hydroxide (24 mg, 1.9 mmol) in THFmethanolwater (2:1:1, 4 ml) was stirred at room temperature for 3 hours, then acidified with 1M HCl and concentrated to give of 3-[(R)-1-(4-chloro-2-fluoro-3-phenoxy-phenyl)-propylamino]-butyric acid.

### Step 3

The residue from Step 2 was taken up in DMF (5 ml) and 1-ethyl-3-(3-dimethylaminopropyl)carbodiimide (43 mg, 0.74 mmol), 1-hydroxy-7-azabenzotriazole (101 mg, 0.74 mmol) and triethylamine (0.17 ml, 1.24 mmol) were added, followed by methylamine (0.32 ml of a 40% wt. solution in water, 3.72 mmol). The reaction mixture was stirred overnight at room temperature then partitioned between sat. sodium hydrogen carbonate and DCM. The organic fractions were dried over sodium sulfate, filtered and concentrated. The residue was subjected to preparative hplc to yield the (R,R) isomer (Example 131B (29 mg) as a white solid.

### Example 132

### 1-{3-[3-((S)-1-Amino-propyl)-6-chloro-2-fluoro-phenoxy]-phenyl}-ethanone (Example 132A); and 1-{3-[3-((S)-1-Amino-propyl)-6-chloro-2-fluoro-phenoxy]-phenyl}-ethanol.hydrochloride (Example 132B)

### Step 1

The enantiomer of Key Intermediate 3a (1.5 g, 4.9 mmol) was coupled with 3-iodophenylboronic acid (2 g) as described in Key Intermediate 1, step 1 to generate (S)-2-methyl-propane-2-sulfinic acid {(S)-1-[4-chloro-2-fluoro-3-(3-iodo-phenoxy)-phenyl]-propyl}-amide (649 mg) as a colourless gum. MS: [M+H]⁺508.

### Step 2

(S)-2-methyl-propane-2-sulfinic acid {(S)-1-[4-chloro-2-fluoro-3-(3-iodo-phenoxy)-phenyl]-propyl}-amide (640 mg, 1.2 mmol), lithium chloride (160 mg, 3.8 mmol) and tetrakis(triphenylphosphine)palladium (0) (145 mg, 0.12 mmol) in acetonitrile (3 ml) was added tributyl-(1-ethoxyvinyl)-tin (0.47 ml, 1.4 mmol). The reaction mixture was heated for 30 mins under microwave irradiation, then filtered and concentrated. The residue was purified by column chromatography, eluting with 30-40% ethyl acetate in petrol to give (S)-2-methyl-propane-2-sulfinic acid {(S)-1-[4-chloro-2-fluoro-3-[(3-(1-ethoxyvinyl)-phenoxy]-phenyl]-propyl}-amide (287 mg) as a yellow oil. MS: [M+H]⁺454.

### Step 3

(S)-2-methyl-propane-2-sulfinic acid {(S)-1-[4-chloro-2-fluoro-3-[(3-(1-ethoxyvinyl)-phenoxy]-phenyl]-propyl}-amide (287 mg, 0.63 mmol) was dissolved in dioxane (3 ml) and 2M HCl (3 ml) was added. The reaction was stirred at room temperature for 1 hour, then concentrated to afford (S)-1-[4-chloro-2-fluoro-3-[3-acetyl-phenoxy]-phenyl]-propyl-amine (Example 132A), which was used without further purification. MS: [M+H]⁺322.

### Step 4

A solution of (S)-1-[4-chloro-2-fluoro-3-[(3-acetyl-phenoxy]-phenyl]-propyl-amine and sodium borohydride (80 mg, 2.1 mmol) in methanol (5 ml) was stirred for 1 hour then concentrated. The residue was partitioned between sat. ammonium chloride and DCM and the organic fractions dried over sodium sulfate, filtered and evaporated to dryness. The crude material was purified by preparative hplc to yield the title compound product (Example 132B) (59 mg) as a white solid.

### Example 133

### {3-[3-((S)-1-Amino-propyl)-6-chloro-2-fluoro-phenoxy]-phenyl}-pyrrolidin-1-yl-methanone.hydrochloride

### Step 1

The enantiomer of Key Intermediate 3a (1.5 g, 4.9 mmol) was coupled with 3-methoxy-carbonyl-phenylboronic acid (2.2 g) as described in Key Intermediate 1, step 1 to generate (S)-3-{6-chloro-2-fluoro-3-[(S)-1-(2-methyl-propane-2-sulfinylamino)-propyl]-phenoxy}-benzoic acid methyl ester (1.3 g) as a pale yellow foam. MS: [M+H]⁺442.

### Step 2

(S)-3-{6-Chloro-2-fluoro-3-[(S)-1-(2-methyl-propane-2-sulfinylamino)-propyl]-phenoxy}-benzoic acid methyl ester was treated with lithium hydroxide and coupled with pyrrolidine using the method described in Example 131, step 2 to generate (S)-3-{6-chloro-2-fluoro-3-[(S)-1-(2-methyl-propane-2-sulfinylamino)-propyl]-phenoxy}-benzoic acid pyrrolidine amide (134 mg) as a colourless foam. MS: [M+H]⁺481.

### Step 3

(S)-3-{6-Chloro-2-fluoro-3-[(S)-1-(2-methyl-propane-2-sulfinylamino)-propyl]-phenoxy}-benzoic acid pyrrolidine amide was hydrolysed with HCl as described in Key Intermediate 1, step 6 to give the title compound (15 mg) as a white solid.

### Example 134

### 3-[(R)-1-(4-Chloro-2-fluoro-3-phenoxy-phenyl-propylamino]-pentanenitrile (Example 134A); and (S)-3-[(R)-1-(4-Chloro-2-fluoro-3-phenoxy-phenyl)-propylamino]-pentanoic acid amide.hydrochloride (Example 134B)

### Step 1

(R)-1-(2-Chloro-4-fluoro-3-phenoxy-phenyl)-propylamine (prepared analogously to Key Intermediate 1) (186 mg, 0.67 mmol) was reductively aminated with 3-oxopentanenitrile using the method described in Example 56, step 1. The 3-[(R)-1-(4-chloro-2-fluoro-3-phenoxy-phenyl)-propylamino]-pentanenitrile thus produced was used in the next step as a mixture of diastereomers. MS: [M+H]⁺361.

### Step 2

A solution of crude 3-[(R)-1-(4-chloro-2-fluoro-3-phenoxy-phenyl)-propylamino]-pentanenitrile (0.67 mmol, assumed) in ethanol (5 ml) was cooled to 0° C. and 1M sodium hydroxide (2.5 ml, 2.5 mmol) was added followed by hydrogen peroxide (7 ml of a 30% aqueous solution). The resulting mixture was stirred for 5 hours at 0° C., then at room temperature overnight. The mixture was cooled back to 0° C. and sat. sodium thiosulphate (15 ml) was added dropwise. The ethanol was removed in vacuo and the remaining solution extracted into DCM. The organic fractions were dried over Na₂SO₄, filtered and concentrated and the residue purified by preparative hplc to afford the title compound (S,R) isomer (24 mg) as a white solid. Further elution yielded the (R,R) isomer (26 mg) as a white solid.

### Example 136

### [(S)-1-(2,4-Difluoro-3-phenoxy-phenyl)-propyl]-(2,3-dihydro-1H-isoindol-4-yl)-amine.hydrochloride

### Step 1

A solution of Key Intermediate 1 (50 mg, 0.19 mmol), tert-butyl 4-bromoisoindoline-2-carboxylate (57 mg, 0.19 mmol) and sodium tert-butoxide (26 mg, 0.27 mmol) in dioxane (1 ml) was degassed by bubbling through nitrogen for 5 mins. Tris(dibenzylideneacetone)dipalladium (0) (5 mg) and 2,2′-bis(diphenylphosphino)-1,1′-binapthyl (5 mg) were added and the reaction mixture was heated to 120° C. for 20 mins under microwave irradiation. The mixture was partitioned between sat. sodium hydrogen carbonate and ethyl acetate. The organic fractions were washed with brine, dried over magnesium sulfate, filtered and concentrated. The residue was purified by preparative hplc to give 4-[(S)-1-(2,4-difluoro-3-phenoxy-phenyl)-propylamino]-1,3-dihydro-isoindole-2-carboxylic acid tert-butyl ester (23 mg) as a solid.

### Step 2

4-[(S)-1-(2,4-Difluoro-3-phenoxy-phenyl)-propylamino]-1,3-dihydro-isoindole-2-carboxylic acid tert-butyl ester (23 mg, 0.05 mmol) was dissolved in a saturated solution of HCl in ethyl acetate (2 ml) and stirred at room temperature overnight. The solution was evaporated to dryness to yield the title compound (12 mg) as a white solid.

### Example 138

### 3-[3-((S)-1-Amino-propyl)-6-chloro-2-fluoro-phenoxy]-N-(1-benzyl-1H-pyrazol-4-ylmethyl)-N-methyl-benzamide.hydrochloride

### Step 1

Triethylamine (0.45 ml, 3.2 mmol) was added to a solution of 1-benzyl-4-formyl pyrrole (300 mg, 1.6 mmol) and methylamine hydrochloride (217 mg, 3.2 mmol) in DCE (6 ml). The resulting solution was stirred for 4 hours at room temperature before sodium borohydride (122 mg, 3.2 mmol) was added and the reaction stirred overnight. The mixture was partitioned between sat. ammonium chloride and DCM and the combined organic fractions were dried over sodium sulfate, filtered and evaporated. The residue was purified by preparative hplc to afford (1-benzyl-1H-pyrazol-4-ylmethyl)-methyl-amine (125 mg) as a colourless oil. MS: [M+H]⁺202.

### Step 2

(1-Benzyl-1H-pyrazol-4-ylmethyl)-methyl-amine (103 mg) and 3-{6-chloro-2-fluoro-3-[(S)-1-((S)-2-methyl-propane-2-sulfinylamino)-propyl]-phenoxy}-benzoic acid (110 mg) were coupled as described in Example 133 step 2 to give 3-[3-(-[(S)-1-(2-methyl-propane-2-sulfinylamino)-propyl])-6-chloro-2-fluoro-phenoxy]-N-(1-benzyl-1H-pyrazol-4-ylmethyl)-N-methyl-benzamide (118 mg) as a white foam. MS: [M+H]⁺611.

### Step 3

3-[3-(-[(S)-1-(2-Methyl-propane-2-sulfinylamino)-propyl])-6-chloro-2-fluoro-phenoxy]-N-(1-benzyl-1H-pyrazol-4-ylmethyl)-N-methyl-benzamide was dissolved in a sat. solution of HCl in ethyl acetate (3 ml) and stirred at room temperature for 1 hour. The resulting suspension was filtered and the solid washed with ethyl acetate and dried to yield the title compound (65 mg) as a white solid.

### Example 139

### Ethyl-carbamic acid (R)-1-{3-[3-((S)-1-amino-propyl)-6-chloro-2-fluoro-phenoxy]-phenyl}-ethyl ester.hydrochloride

### Step 1

To a solution of (S)-2-methyl-propane-2-sulfinic acid {(S)-1-[4-chloro-2-fluoro-3-(3-formyl-phenoxy)-phenyl]-propyl}-amide (prepared as described in Example 111) (627 mg, 1.5 mmol) in THF (8 ml) at −78° C. was added methyl magnesium bromide (3.8 ml of a 1M solution in THF, 3.8 mmol). After stirring for 1 hour at this temperature, further methyl magnesium bromide (2.3 ml of a 1M solution in THF, 2.3 mmol) was added. After a further 1 hour at this temperature, the reaction was quenched by addition of sat. ammonium chloride and extracted into DCM. The combined organic fractions were dried over sodium sulfate, filtered and concentrated and the residue purified by column chromatography. Elution with 0-60% ethyl acetate in petrol gave of (S-2-methyl-propane-2-sulfinic acid {(S)-1-[4-chloro-2-fluoro-3-[3-(2-hydroxyethyl)-phenoxy]-phenyl]-propyl}-amide (184 mg) as a colourless gum, which was used as a mixture of diastereomers. MS: [M+H−H₂O] 410.

### Step 2

Ethyl isocyanate (0.037 ml, 0.47 mmol) was added to a solution of (S-2-methyl-propane-2-sulfinic acid {(S)-1-[4-chloro-2-fluoro-3-[3-(2-hydroxyethyl)-phenoxy]-phenyl]-propyl}-amide (184 mg, 0.43 mmol) and triethylamine (0.06 ml, 0.43 mmol) in DCM. The reaction was stirred for 24 hours and ethyl isocyanate (0.037 ml, 0.47 mmol) was added. After 48 hours, further ethyl isocyanate (0.037 ml, 0.47 mmol) was added. The reaction was stirred 48 hours more, before being diluted with DCM, washed with water, dried over sodium sulfate, filtered and concentrated. The crude material was purified by preparative hplc to yield ethyl-carbamic acid (R)-1-(3-{6-chloro-2-fluoro-3-[(S)-1-((S)-2-methyl-propane-2-sulfinylamino)-propyl]-phenoxy}-phenyl)-ethyl ester (60 mg) as a beige oil. MS: [M+H]⁺499. Further elution provided the (S,S,S) isomer (54 mg) also as a beige oil. MS: [M+H]⁺499.

### Step 3

Ethyl-carbamic acid (R)-1-(3-{6-chloro-2-fluoro-3-[(S)-1-((S)-2-methyl-propane-2-sulfinylamino)-propyl]-phenoxy}-phenyl)ethyl ester (60 mg) was hydrolysed with HCl as described in Key Intermediate 1, step 6 to give the title compound (22 mg) as a white solid.

### Example 141

### 3-[3-((S)-1-Amino-propyl)-6-chloro-2-fluoro-phenoxy]-N-methyl-N-(1H-pyrazol-4-ylmethyl)-benzamide.hydrochloride

3-[3-((S)-1-Amino-propyl)-6-chloro-2-fluoro-phenoxy]-N-(1-benzyl-1H-pyrazol-4-ylmethyl)-N-methyl-benzamide (prepared as described in Example 138) (65 mg, 0.13 mmol) was dissolved in methanol (4 ml) and palladium hydroxide (2 mg, 0.013 mmol) and HCl (0.033 ml of a 4M solution in dioxane, 0.13 mmol) were added. The resulting mixture was stirred under a hydrogen atmosphere for 16 hours, then filtered and concentrated. The residue was purified by preparative hplc to afford the title compound (26 mg) as a white solid.

### Example 144

### [(S)-1-(2,4-Difluoro-3-phenoxy-phenyl)-propyl]-pyridin-4-yl-amine. hydrochloride

A solution of Key Intermediate 1 hydrochloride (100 mg, 0.38 mmol) and 4-chloropyridine hydrochloride (55 mg, 0.38 mmol) in DCM (5 ml) was washed with sat. sodium hydrogen carbonate, dried over sodium sulfate, filtered and evaporated to dryness. The residue was dissolved in NMP (1 ml) and heated under microwave irradiation for 10 mins at 170° C., followed by 10 mins at 185° C. The material was purified by preparative hplc to afford the title compound (6 mg) as a light brown foam.

### Example 145

### [(R)-1-(4-Chloro-2-fluoro-3-phenoxy-phenyl)-propyl]-[(S)-1-(1H-pyrazol-4-yl)-ethyl]-amine. dihydrochloride (Example 145A); and [(R)-1-(4-Chloro-2-fluoro-3-phenoxy-phenyl)-propyl]-[(R)-1-(1H-pyrazol-4-yl)-ethyl]-amine.dihydrochloride (Example 1458)

### Step 1

(R)-1-(2-Chloro-4-fluoro-3-phenoxy-phenyl)-propylamine (prepared analogously to Key Intermediate 1) (89 mg, 0.32 mmol) was reductively aminated with 1-[1-(4-methylbenzenesulphonyl)-1H-pyrazol-4-yl]ethan-1-one using the method described in Example 3, step 2 to give [(R)-1-(4-chloro-2-fluoro-3-phenoxy-phenyl)-propyl]-{1-[1-(toluene-4-sulfonyl)-1H-pyrazol-4-yl]-ethyl}-amine as a mixture of diastereomers which was used in the next step. MS: [M+H]⁺528.

### Step 2

[(R)-1-(4-Chloro-2-fluoro-3-phenoxy-phenyl)-propyl]-{1-[1-(toluene-4-sulfonyl)-1H-pyrazol-4-yl]-ethyl}-amine (90 mg, 0.17 mmol) was dissolved in a 4M solution of HCl in dioxane (5 ml) and heated to 80° C. for 1 hour. The resulting solid was separated by filtration and washed with dioxane to afford the (S,R) isomer of the title compound (40 mg) as a white solid. The filtrate was concentrated and purified by preparative hplc to yield the (R,R) isomer of the title compound (10 mg) also as a white solid.

### Example 156

### 6-[3-((R)-1-Amino-propyl)-6-chloro-2-fluoro-phenoxy]-pyridin-3-ylamine.dihydrochloride

### Step 1

A suspension of Key Intermediate 3 (200 mg, 0.65 mmol), 2-fluoro-5-nitropyridine (92 mg, 0.65 mmol) and potassium carbonate (225 mg, 1.6 mmol) in DMSO (2 ml) was stirred at room temperature overnight. The mixture was partitioned between brine and diethyl ether and the organic fraction dried over sodium sulfate, filtered and concentrated to give 2-methyl-propane-2-sulfinic acid {(R)-1-[3-(5-nitro-pyridin-2-yloxy)-4-chloro-2-fluoro-phenyl]-propyl}-amide (260 mg) as a colourless oil. MS: [M+H]⁺430.

### Step 2

2-Methyl-propane-2-sulfinic acid {(R)-1-[3-(5-nitro-pyridin-2-yloxy)-4-chloro-2-fluoro-phenyl]-propyl}-amide was reduced as described in Example 19, step 2 to generate 2-methyl-propane-2-sulfinic acid {(R)-1-[3-(5-amino-pyridin-2-yloxy)-4-chloro-2-fluoro-phenyl]-propyl}-amide (100 mg) as a colourless gum. MS: [M+H]⁺400.

### Step 3

2-Methyl-propane-2-sulfinic acid {(R)-1-[3-(5-amino-pyridin-2-yloxy)-4-chloro-2-fluoro-phenyl]-propyl}-amide was hydrolysed as described in Key Intermediate 1, step 6 to generate the title compound (67 mg) as a white solid.

### Example 163

### [(R)-1-(4-Chloro-2-fluoro-3-phenoxy-phenyl)-propyl]-1-[(R)-1-(1H-pyrrol-3-yl)-ethyl]-amine.dihydrochloride

### Step 1

(R)-1-(2-Chloro-4-fluoro-3-phenoxy-phenyl)-propylamine (prepared analogously to Key Intermediate 1) (200 mg, 0.76 mmol) was suspended in toluene (20 ml). 3-Acetyl-1-tosyl-pyrrole (167 mg, 0.76 mmol) was added, followed by tosic acid (5 mg, cat.) and the resulting mixture heated to reflux for 48 hours. The reaction was evaporated to dryness and the residue, [(R)-1-(4-chloro-2-fluoro-3-phenoxy-phenyl)-propyl]-[1-[1-(toluene-4-sulfonyl)-1H-pyrrol-3-yl]-eth-(E)-ylidene]-amine, (407 mg) used without further purification.

### Step 2

[(R)-1-(4-Chloro-2-fluoro-3-phenoxy-phenyl)-propyl]-[1-[1-(toluene-4-sulfonyl)-1H-pyrrol-3-yl]-eth-(E)-ylidene]-amine (0.76 mmol, assumed) was dissolved in methanol (10 ml) and cooled to 0° C. Sodium borohydride (24 mg, 0.76 mmol) was added and the reaction was stirred for 1 hour at 0° C., followed by 15 mins at room temperature. The mixture was concentrated and the residue partitioned between sat. sodium hydrogen carbonate and ethyl acetate. The combined organic fractions were dried over magnesium sulfate, filtered, evaporated and purified by preparative hplc to afford [(R)-1-(4-chloro-2-fluoro-3-phenoxy-phenyl)-propyl]-[(R)-1-[1-(toluene-4-sulfonyl)-1H-pyrrol-3-yl]-ethyl]-amine (55 mg). MS: [M+H]⁺527. Further elution yielded the (R,S) isomer (32 mg). MS: [M+H]⁺527.

### Step 3

A solution of [(R)-1-(4-chloro-2-fluoro-3-phenoxy-phenyl)-propyl]-[(R)-1-[1-(toluene-4-sulfonyl)-1H-pyrrol-3-yl]-ethyl]-amine (55 mg, 0.10 mmol) in methanol (2 ml) was added to a suspension of magnesium turnings (50 mg, 2.0 mmol) in methanol (2 ml) and the resulting mixture stirred at room temperature for 3 hours. The mixture was filtered, then partitioned between sat. ammonium chloride and ethyl acetate. The organic fractions were dried over magnesium sulfate, filtered, concentrated and purified by preparative hplc to afford the title compound (23 mg) as a solid.

### Example 171

### 1-{3-[3-((R)-1-Amino-propyl)-6-chloro-2-fluoro-phenoxy]-phenyl}-3-(2,2,2-trifluoro-ethyl)-urea.hydrochloride

### Step 1

Trifluoroethylamine (100 ml) and tetrahydrofuran (250 ml) were charged to a reaction vessel, stirring under nitrogen. Ice cooling was applied and neat 3-bromophenylisocyanate (50 g, 0.25 mol) was charged dropwise over 30 mins, maintaining the temperature at 15° C. Line rinse tetrahydrofuran (62 ml). The reaction was allowed to warm to RT overnight, with stirring under nitrogen. LC-MS (basic) indicated the presence of product. The reaction mixture was concentrated in vacuo at 40° C. to give 1-(3-bromo-phenyl)-3-(2,2,2-trifluoro-ethyl)-urea (78.05 g) used without further purification.

### Step 2

1-(3-Bromo-phenyl)-3-(2,2,2-trifluoro-ethyl)-urea (78 g, 0.26 mol), bis(pinacolato)-diboron (133.3 g, 0.53 mol) and potassium acetate (77.3 g, 0.79 mol) were charged to a reaction vessel, under nitrogen. DMSO (anhydrous, 275 ml) was charged to the reaction vessel by syringe and the thick mixture was stirred whilst degassing with vacuumnitrogen (×3). PdCl₂(dppf) solid (19.2 g, 26.2 mmol) was charged and then the thick mixture was stirred whilst degassing with vacuumnitrogen (×3). Heat was applied (oil set temperature 100° C.) and heating was maintained overnight, stirring under nitrogen. LC-MS (acidic) indicated the presence of product. The reaction was cooled to RT and diluted with water (800 ml), extracted with ethyl acetate (2×800 ml). The combined organics were washed with water (800 ml), brine (800 ml), dried (magnesium sulfate) and concentrated in vacuo at 40° C. The resulting black residue was triturated with petrol (800 ml) and ethyl acetate (40 ml), with vigorous stirring. The slurry was filtered, cake wash petrol (400 ml) and air dried to give 1-[3-(4,4,5,5-tetramethyl-[1,3,2]dioxaborolan-2-yl)-phenyl]-3-(2,2,2-trifluoro-ethyl)-urea (91.2 g) as a greybrown solid.

### Step 3

Sodium periodate (46.5 g, 218 mmol) was added to a solution of 1-[3-(4,4,5,5-tetramethyl-[1,3,2]dioxaborolan-2-yl)-phenyl]-3-(2,2,2-trifluoro-ethyl)-urea (25 g, 72.6 mmol) in THFwater (4:1, 250 ml). The reaction was stirred for 30 mins before HCl (51 ml of a 1M solution, 51 mmol) was added and the resulting mixture stirred 3 hours further. The solution was diluted with water and extracted with ethyl acetate. The combined organic fractions were washed with 10% sodium thiosulphate and brine, dried over magnesium sulfate, filtered and concentrated. The residue was triturated with diethyl ether and dried to afford 3-(2,2,2-trifluoro-ethyl)-ureido-phenyl-boronic acid (16.50 g) as a grey powder. MS: [M+H]⁺263.

### Step 4

Key Intermediate 3 was treated with 3-(2,2,2-trifluoro-ethyl)-ureido-phenyl-boronic acid as described in Example 132, steps 1 and 3 to generate the title compound (77 mg) as a white solid.

### Example 184 and 188

### (R)-1-{4-Chloro-2-fluoro-3-[4-(1-methoxy-ethyl)-phenoxy]-phenyl}-propylamine

### Step 1

Key Intermediate 3 (300 mg g, 0.98 mmol) was coupled with 4-acetylphenylboronic acid (328 mg) as described in Key Intermediate 1, step 1 to generate (R)-2-methyl-propane-2-sulfinic acid {(R)-1-[3-(4-acetyl-phenoxy)-4-chloro-2-fluoro-phenyl]-propyl}-amide (300 mg) as a brown oil. MS: [M+H]⁺426.

### Step 2

Sodium borohydride (54 mg, 1.41 mmol) was added to a solution of (R)-2-methyl-propane-2-sulfinic acid {(R)-1-[3-(4-acetyl-phenoxy)-4-chloro-2-fluoro-phenyl]-propyl}-amide (300 mg, 0.71 mmol) at 0° C. and the resulting solution was stirred for 1 hour at this temperature. The reaction was quenched with sat. ammonium chloride and extracted into DCM. Combined organic fractions were dried over sodium sulfate, filtered and evaporated. The residue was purified by column chromatography, eluting with 0-100% ethyl acetate in petrol to afford (R)-2-methyl-propane-2-sulfinic acid {(R)-1-[3-(4-[1-hydroxyethyl]-phenoxy)-4-chloro-2-fluoro-phenyl]-propyl}-amide (167 mg) as a colourless foam. MS: [M+H−H₂O]⁺410.

### Step 3

(R)-2-methyl-propane-2-sulfinic acid {(R)-1-[3-(4-[1-hydroxyethyl]-phenoxy)-4-chloro-2-fluoro-phenyl]-propyl}-amide (167 mg, 0.39 mmol) was treated with HCl as described in Key Intermediate 1, step 6. Purification by preparative hplc afforded 1-{4-[3-((R)-1-amino-propyl)-6-chloro-2-fluoro-phenoxy]-phenyl}-ethanol (4 mg) as a white solid. Further elution afforded (R)-1-{4-chloro-2-fluoro-3-[4-(1-methoxy-ethyl)-phenoxy]-phenyl}-propylamine (115 mg) also as a white solid.

### Example 190

### Cyclopropylmethyl-carbamic acid 5-[3-((R)-1-amino-propyl)-6-chloro-2-fluoro-phenoxy]-2-fluoro-benzyl ester.hydrochloride

### Step 1

(R)-2-Methyl-propane-2-sulfinic acid {(R)-1-[4-chloro-2-fluoro-3-(4-fluoro-3-hydroxymethyl-phenoxy)-phenyl]-propyl}-amide (prepared analogously to Example 111, using 4-fluoro-3-formylphenylboronic acid in step 1) (145 mg, 0.34 mmol) was added to a suspension of carbonyl diimidazole (54 mg, 0.34 mmol) in THF (5 ml) at 10° C. The reaction was stirred for 2 hours at room temperature, before cyclopropanemethylamine (24 mg, 0.34 mmol), triethylamine (0.047 ml, 0.34 mmol) and 1,8-diazabicycloundec-7-ene (0.05 ml, 0.34 mmol) were added. The resulting mixture was stirred at room temperature overnight, before being diluted with DCM and washed with water. The organic layer was dried over sodium sulfate, filtered and concentrated and the residue purified by preparative hplc to afford cyclopropylmethyl-carbamic acid 5-{6-chloro-2-fluoro-3-[(R)-1-((R)-2-methyl-propane-2-sulfinylamino)-propyl]-phenoxy}-2-fluoro-benzyl ester (64 mg) as a colourless oil. MS: [M+H]⁺529.

### Step 2

Cyclopropylmethyl-carbamic acid 5-{6-chloro-2-fluoro-3-[(R)-1-((R)-2-methyl-propane-2-sulfinylamino)-propyl]-phenoxy}-2-fluoro-benzyl ester (64 mg, 0.12 mmol) was treated with HCl as described in Key Intermediate 1, step 6 to generate the title compound (36 mg) as a white solid. MS: [M+H]⁺425.

### Example 203

### (R)-1-[4-Chloro-2-fluoro-3-(4-oxazol-5-yl-phenoxy)-phenyl]-propylamine.hydrochloride

### Step 1

Key Intermediate 3 (500 mg, 1.63 mmol) was coupled with 4-formylphenyl boronic acid as described in Key Intermediate 1, step 1 to generate (R)-2-methyl-propane-2-sulfinic acid {(R)-1-[4-chloro-2-fluoro-3-(4-formyl-phenoxy)-phenyl]-propyl}-amide (477 mg) as a colourless oil. MS: [M+H]⁺412.

### Step 2

A mixture of (R)-2-methyl-propane-2-sulfinic acid {(R)-1-[4-chloro-2-fluoro-3-(4-formyl-phenoxy)-phenyl]-propyl}-amide (234 mg, 0.57 mmol), (4-toluene-sulfonyl)methyl isocyanide (111 mg, 0.57 mmol) and potassium carbonate (102 mg, 0.74 mmol) in methanol (8 ml) was heated to reflux for 2 hours, then concentrated. The residue was taken up in DCM and washed with water. The aqueous fraction was further extracted into DCM and the combined organic layers were dried over sodium sulfate, filtered and concentrated. The residue was purified by column chromatography to give R)-2-methyl-propane-2-sulfinic acid {(R)-1-[4-chloro-2-fluoro-3-(4-oxazol-5-yl-phenoxy)-phenyl]propyl}-amide (210 mg) as a colourless oil. MS: [M+H]⁺451.

### Step 3

(R)-2-Methyl-propane-2-sulfinic acid {(R)-1-[4-chloro-2-fluoro-3-(4-oxazol-5-yl-phenoxy)-phenyl]propyl}-amide (210 mg, 0.47 mmol) was treated with HCl as described in Key Intermediate 1, step 6 to afford the title compound (130 mg) as a white solid.

### Example 204

### 4-[3-((R)-1-Amino-propyl)-6-chloro-2-fluoro-phenoxy]-benzaldehyde oxime.hydrochloride

### Step 1

A solution of hydroxylamine hydrochloride (49 mg, 0.7 mmol) in water (1 ml) was added dropwise to a solution of (R)-2-methyl-propane-2-sulfinic acid {(R)-1-[4-chloro-2-fluoro-3-(4-formyl-phenoxy)-phenyl]-propyl}-amide (prepared as described in Example 203) (243 mg, 0.6 mmol) and sodium carbonate (125 mg, 1.2 mmol) in ethanolwater (1:1, 4 ml). The resulting mixture was stirred for 4 hours, diluted with water and filtered. The solid was washed with water and dried to yield (R)-2-Methyl-propane-2-sulfinic acid ((R)-1-{4-chloro-2-fluoro-3-[4-(hydroxyimino-methyl)-phenoxy]-phenyl}-propyl)-amide (147 mg) as a white solid. MS: [M+H]⁺427.

### Step 2

(R)-2-Methyl-propane-2-sulfinic acid ((R)-1-{4-chloro-2-fluoro-3-[4-(hydroxyimino-methyl)-phenoxy]-phenyl}-propyl)-amide (147 mg, 0.35 mmol) was treated with HCl as described in Key Intermediate 1, step 6 to afford the title compound (104 mg) as a white solid.

### Example 218

### 4-[3-((R)-1-Amino-propyl)-6-chloro-2-fluoro-phenoxy]-1H-pyridin-2-one. hydrochloride

### Step 1

Key Intermediate 3 (300 mg, 0.97 mmol) was coupled with 2-methoxy-4-pyridinylboronic acid (374 mg) as described in Key Intermediate 1, step 1 to generate (R)-2-methyl-propane-2-sulfinic acid {(R)-1-[4-chloro-2-fluoro-3-(2-methoxy-pyridin-4-yloxy)-phenyl]-propyl}-amide (250 mg) as a colourless oil. MS: [M+H]⁺415.

### Step 2

(R)-2-Methyl-propane-2-sulfinic acid {(R)-1-[4-chloro-2-fluoro-3-(2-methoxy-pyridin-4-yloxy)-phenyl]-propyl}-amide (250 mg, 0.60 mmol) was heated to reflux overnight in 6N HCl (5 ml). The reaction was evaporated to dryness and coevaporated twice further with toluene. The residue was triturated with diethyl ether to afford the title compound (191 mg) as a colourless powder.

### Example 223

### (R)—N-(2-Amino-ethyl)-3-[(R)-1-(4-chloro-2-fluoro-3-phenoxy-phenyl)-propylamino]-butyramide.dihydrochloride

A solution of 3-[(R)-1-(4-chloro-2-fluoro-3-phenoxy-phenyl)-propylamino]-butyric acid (prepared as described in Example 28) (100 mg, 0.27 mmol) was coupled with tert-butyl N-(2-aminoethyl)carbamate (219 mg) as described in Example 28. The crude product was taken up in 1,4-dioxane (2 ml) and HCl (5 ml of a 4M solution in 1,4-dioxane) was added. The resulting solution was stirred for 1.5 hours, then concentrated. The residue purified by preparative hplc and subsequent HCl salt formation afforded the (R,R) isomer title compound (26 mg) as a white solid. Further elution and subsequent HCl salt formation afforded the (S,R) isomer (24 mg) also as a white solid.

### Example 225

### [2,4-Difluoro-3-(3-methyl-4-nitro-phenoxy)-benzyl]-pyridin-4-yl-amine (Example 225A); and [3-(4-Amino-3-methyl-phenoxy)-2,4-difluoro-benzyl]-pyridin-4-yl-amine. hydrochloride (Example 225B)

### Step 1

2,4-Difluoro-3-methoxybenzoic acid (5 g, 26.6 mmol) was dissolved in thionyl chloride (26.6 mL) and heated at 80° C. for 4 hours before excess thionyl chloride was evaporated. The residue was dissolved in THF (53.2 mL) cooled to 0° C. and treated with 2-aminopyridine (3 g, 31.9 mmol) in portions followed by the addition of pyridine (6.45 mL, 79.7 mmol). The mixture was allowed to warm to room temperature and was stirred overnight. Saturated sodium hydrogen carbonate solution was added and THF and pyridine were evaporated before the aqueous layer was extracted into CHCl3 (×3). The combined organic extracts were dried (sodium sulfate), filtered and concentrated. Column chromatography eluting with a gradient of 0% EtOAcpetrol to 40% EtOAc gave 2.67 g of 2,4-difluoro-3-methoxy-n-pyridin-2-yl-benzamide as a white crystalline solid. MS: [M+H]+ 265.

### Step 2

To a stirred solution of (2,4-difluoro-3-methoxy-benzyl)-pyridin-2-yl-amine (2.67 g, 10.1 mmol) in THF (25.3 mL) at 0° C. was added borane in THF (1M solution, 60.6 mL, 60.6 mmol) dropwise. The mixture was heated at 60° C. for 7 hours. MeOH was added carefully, the mixture stirred for 1 hour then conc. HCl was added carefully and the mixture stirred for 1 hour. The solvents were removed under vacuum. The basic fraction was isolated by passing the residue through an SCX column providing 1.44 g of (2,4-difluoro-3-methoxy-benzyl)-pyridin-2-yl-amine which was used without further purification.

### Step 3

To a stirred solution of (2,4-difluoro-3-methoxy-benzyl)-pyridin-2-yl-amine (1.44 g, 5.75 mmol) in DCM (46 mL) at 0° C. was added boron tribromide (1.11 mL, 11.5 mmol) slowly. The mixture was allowed to warm to room temperature and was stirred overnight. The reaction was cooled to 0° C. quenched by the addition of water and concentrated. The basic fraction was isolated by passing the residue through an SCX column providing 1.15 g of 2,6-difluoro-3-(pyridin-2-ylaminomethyl)-phenol as a white solid. MS: [M+H]+ 237.

### Step 4

A suspension of 2,6-difluoro-3-(pyridin-2-ylaminomethyl)-phenol (0.1 g, 0.423 mmol), 5-fluoro-2-nitrotoluene (0.0797 g, 0.847 mmol) and potassium carbonate (0.117 g, 0.847 mmol) in N-methyl-2-pyrrolidone (0.635 mL) was heated under microwave irradiation at 100° C. for 40 minutes. The mixture was filtered and the solution was subject to preparative HPLC providing [2,4-difluoro-3-(3-methyl-4-nitro-phenoxy)-benzyl]-pyridin-4-yl-amine, 37 mg. MS: [M+H]+ 372.

### Step 5

[2,4-difluoro-3-(3-methyl-4-nitro-phenoxy)-benzyl]-pyridin-4-yl-amine (0.037 g, 0.0996 mmol) was reduced under an atmosphere of hydrogen using the method described in Example 112, step 2. Preparative HPLC provided [3-(4-amino-3-methyl-phenoxy)-2,4-difluoro-benzyl]-pyridin-4-yl-amine, 27 mg.

### Example 227

### 5-[3-((R)-1-Amino-propyl)-6-chloro-2-fluoro-phenoxy]-2-fluoro-benzamide (R isomer).hydrochloride

### Step 1

Key Intermediate 3 (645 mg, 2.1 mmol) was coupled with 4-fluoro-3-methoxycarbonylphenyl boronic acid as described in Key Intermediate 1, step 1 to generate 5-{6-chloro-2-fluoro-3-[(R)-1-((R)-2-methyl-propane-2-sulfinylamino)-propyl]-phenoxy}-2-fluoro-benzoic acid methyl ester (77 mg) as a colourless oil. MS: [M+H]⁺460.

### Step 2

A solution of 5-{6-chloro-2-fluoro-3-[(R)-1-((R)-2-methyl-propane-2-sulfinylamino)-propyl]-phenoxy}-2-fluoro-benzoic acid methyl ester (70 mg, 0.15 mmol) in 7M ammoniamethanol (3 ml) was stirred at room temperature overnight, then concentrated to give 5-{6-chloro-2-fluoro-3-[(R)-1-((R)-2-methyl-propane-2-sulfinylamino)-propyl]-phenoxy}-2-fluoro-benzamide (60 mg), which was used without further purification. MS: [M+H]⁺445.

### Step 3

5-{6-Chloro-2-fluoro-3-[(R)-1-((R)-2-methyl-propane-2-sulfinylamino)-propyl]-phenoxy}-2-fluoro-benzamide (60 mg, 0.14 mmol) was treated with HCl as described in Key Intermediate 1, step 6 to afford the title compound (28 mg) as a white solid.

### Example 229

### R)-3-{(R)-1-[4-Chloro-3-(4-ethynyl-phenoxy)-2-fluoro-phenyl]-propyl-amino}-butyramide.hydrochloride

### Step 1

Key Intermediate 3 (600 mg, 1.95 mmol) was coupled with (4-[(trimethylsilyl)ethynyl]phenyl)boronic acid as described in Key Intermediate 1, step 1 to generate (R)-2-methyl-propane-2-sulfinic acid {(R)-1-[4-chloro-2-fluoro-3-(4-trimethylsilanylethynyl-phenoxy)-phenyl]-propyl}-amide (300 mg). MS: [M+H]⁺480.

### Step 2

(R)-2-Methyl-propane-2-sulfinic acid {(R)-1-[4-chloro-2-fluoro-3-(4-trimethylsilanylethynyl-phenoxy)-phenyl]-propyl}-amide (300 mg, 0.63 mmol) was treated with HCl as described in Key Intermediate 1, step 6 to give (R)-1-[4-chloro-2-fluoro-3-(4-trimethylsilanylethynyl-phenoxy)-phenyl]-propylamine (80 mg) as a solid. MS: [M+H]⁺376.

### Step 3

R)-1-[4-Chloro-2-fluoro-3-(4-trimethylsilanylethynyl-phenoxy)-phenyl]-propylamine (80 mg, 0.21 mmol) was reductively aminated using acetoacetamide and the procedure described in Example 3, step 2 to generate 3-{(R)-1-[4-chloro-2-fluoro-3-(4-trimethylsilanylethynyl-phenoxy)-phenyl]-propylamino}-butyramide (77 mg), which was used in the subsequent step as a mixture of diastereomers. MS: [M+H]⁺461.

### Step 4

To a solution of 3-{(R)-1-[4-chloro-2-fluoro-3-(4-trimethylsilanylethynyl-phenoxy)-phenyl]-propylamino}-butyramide (77 mg, 0.17 mmol) in THF (1 ml) was added tetrabutyl ammonium fluoride (0.17 ml of a 1M solution in THF, 0.17 mmol). The reaction was stirred for 1 hour, then partitioned between sat. ammonium chloride and DCM. The organic fractions were dried over magnesium sulfate, filtered and evaporated to dryness and the residue was purified by preparative hplc to afford the (R,S) isomer of the title compound (9 mg) as a white solid. Further elution yielded the (R,R) isomer title compound (24 mg).

### Example 238

### (R)-1-[4-Chloro-2-fluoro-3-(3-methyl-4-nitro-phenoxy)-phenyl]-propylamine hydrochloride (Example 238A); and (S)—N-(2-Amino-ethyl)-3-{(R)-1-[3-(4-amino-3-methyl-phenoxy)-4-chloro-2-fluoro-phenyl]-propylamino}-butyramide (Example 238B)

### Step 1

(R)-2-Methyl-propane-2-sulfinic acid {(R)-1-[4-chloro-2-fluoro-3-(3-methyl-4-nitro-phenoxy)-phenyl]propyl}-amide (prepared as described in Example 112) (702 mg, 1.59 mmol) was treated with HCl as described in Key Intermediate 1, step 6 to give the product Example 238A) (551 mg) as a yellow solid. MS: [M+H]⁺339.

### Step 2

(R)-1-[4-Chloro-3-(3-methyl-4-nitro-phenoxy)-2-fluoro-phenyl]-propylamine (300 mg, 0.82 mmol) was treated as described in Example 131, step 1 and then as Example 28. The product was purified by column chromatography. Elution with 0-10% iso-propyl alcohol in ethyl acetate afforded [2-((R)-3-{(R)-1-[4-chloro-2-fluoro-3-(3-methyl-4-nitro-phenoxy)-phenyl]-propylamino}-butyrylamino)-ethyl]-carbamic acid tert-butyl ester (77 mg). MS: [M+H]⁺567. Further elution gave the (R,S) isomer (79 mg). MS: [M+H]⁺567.

### Step 3

A mixture of [2-((S)-3-{(R)-1-[4-chloro-2-fluoro-3-(3-methyl-4-nitro-phenoxy)-phenyl]-propylamino}-butyrylamino)-ethyl]-carbamic acid tert-butyl ester (75 mg, 0.132 mmol), iron powder (66 mg, 1.19 mmol) and iron (II) sulfate heptahydrate (81 mg, 0.291 mmol) in dioxanewater (5:1, 6 ml) was heated to reflux for 90 mins. The hot reaction mixture was filtered and the solids washed with dioxane and ethyl acetate. The combined filtrates were concentrated and purified by column chromatography. Elution with 0-20% methanol in ethyl acetate generated [2-((S)-3-{(R)-1-[4-Chloro-2-fluoro-3-(4-amino-3-methyl-phenoxy)-phenyl]-propylamino}-butyrylamino)-ethyl]-carbamic acid tert-butyl ester (67 mg) as a colourless oil. MS: [M+H]⁺537.

### Step 4

[2-((S)-3-{(R)-1-[4-Chloro-2-fluoro-3-(4-amino-3-methyl-phenoxy)-phenyl]-propylamino}-butyrylamino)-ethyl]-carbamic acid tert-butyl ester was hydrolysed with HCl as described in Example 3, step 3 to afford the title compound (56 mg) as a white solid.

### Example 244

### (S)-3-{(R)-1-[3-(4-Acetylamino-3-methyl-phenoxy)-4-chloro-2-fluoro-phenyl]-propylamino}-butyramide.hydrochloride

### Step 1

A solution of (R)-2-Methyl-propane-2-sulfinic acid {(R)-1-[4-chloro-2-fluoro-3-(4-amino-3-methyl-phenoxy)-phenyl]-propyl}-amide (prepared as described in Example 112) (110 mg, 0.267 mmol), acetyl chloride (19 μl, 0.267 mmol) and triethylamine (74 μl, 0.534 mmol) in DCM (4 ml) was stirred for 1 hour at room temperature, before 1M sodium hydrogen carbonate was added. The aqueous fraction was extracted into DCM and the organic fractions were dried, filtered and concentrated to afford N-(4-{6-Chloro-2-fluoro-3-[(R)-1-((R)-2-methyl-propane-2-sulfinylamino)-propyl]-phenoxy}-2-methyl-phenyl)-acetamide (114 mg) as an off-white foam. MS: {M+H]⁺ 455.

### Step 2

N-(4-{6-Chloro-2-fluoro-3-[(R)-1-((R)-2-methyl-propane-2-sulfinylamino)-propyl]-phenoxy}-2-methyl-phenyl)-acetamide was hydrolysed with HCl and then reductively aminated with acetoacetamide using the procedures described in Example 3. The product was purified by column chromatography. Elution with 0-20% ethanol in ethyl acetate gave the (S,R) isomer title compound as a white solid. The mixed fraction was columned again, eluting with 10-20% methanol in ethyl acetate to afford the corresponding (R,R) isomer as a white solid.

### Example 248

### 5-{3-[(R)-1-((R)-2-Carbamoyl-1-methyl-ethylamino)-propyl]-6-chloro-2-fluoro-phenoxy}-pyridine-2-carboxylic acid amide.hydrochloride

### Step 1

Key Intermediate 3 was treated with 2-cyano-5-chloropyridine as described in Example 112, step 1 to provide (R)-2-methyl-propane-2-sulfinic acid {(R)-1-[4-chloro-2-fluoro-3-(6-cyano-pyridin-3-yloxy)-phenyl]propyl}-amide (281 mg) as an off-white solid. MS: [M+H]⁺410.

### Step 2

A solution of (R)-2-methyl-propane-2-sulfinic acid {(R)-1-[4-chloro-2-fluoro-3-(6-cyano-pyridin-3-yloxy)-phenyl]propyl}-amide (281 mg) in 2M HCl in ethyl acetate (2 ml) was stirred at room temperature for 6 hours. The solvent was decanted off and the residue dried under reduced pressure and triturated with ethyl acetate to give 5-[3-((R)-1-amino-propyl)-6-chloro-2-fluoro-phenoxy]-pyridine-2-carboxylic acid amide (271 mg) as a yellow solid. MS: [M+H]⁺324.

### Step 3

5-[3-((R)-1-Amino-propyl)-6-chloro-2-fluoro-phenoxy]-pyridine-2-carboxylic acid amide (271 mg, 0.76 mmol) was reductively aminated with acetoacetamide as described in Example 3, step 2. The product was purified by preparative hplc to afford the (R,R) isomer title compound (48 mg) as a white solid. Further elution yielded the corresponding (R, S) isomer (9 mg) also as a white solid.

### Example 261

### (S)-1-(2,4-Dichloro-3-phenoxy-phenyl)-propylamine.hydrochloride

### Step 1

A solution of 2,6-dichloro-3-methylphenol (5.0 g, 28.2 mmol) and acetic anhydride (5.0 ml, 53 mmol) in pyridine (10 ml) was stirred at room temperature overnight, then concentrated. The residue was partitioned between diethyl ether and 2M HCl. The organic fraction was washed with sodium hydrogen carbonate, dried over sodium sulfate, filtered and evaporated to leave acetic acid 2,6-dichloro-3-methylphenyl ester (5.97 g) which was used without further purification. MS: [M+H]⁺519.

### Step 2

Acetic acid 2,6-dichloro-3-methylphenyl ester (5.95 g, 26.9 mmol) was treated with NBS followed by silver nitrate, as described in Key Intermediate 1, steps 2 and 3, alternative procedure to form 2,4-dichloro-3-acetoxybenzaldehyde (6.3 g) as an impure orange solid. MS: [M+H]⁺233.

### Step 3

2M Sodium hydroxide (60 ml, 120 mmol) was added to a solution of 2,4-dichloro-3-acetoxybenzaldehyde (6.0 g, 25.8 mmol) in methanol (60 ml) and the resulting solution was heated to 50° C. for 2 hours. 2M HCl (80 ml) and water (50 ml) were added and the resulting precipitate was separated by filtration, washed with water and dried to afford 2,4-dichloro-3-hydroxybenzaldehyde (4.085 g) as a cream solid. MS: [M−H]⁻ 189.

### Step 4

2,4-Dichloro-3-hydroxybenzaldehyde (1.0 g, 5.23 mmol) was treated as described in Key Intermediate 1, step 1, alternative procedure to give 2,4-dichloro-3-phenoxybenzaldehyde (380 mg) as an impure fawn solid.

### Step 5

2,4-Dichloro-3-phenoxybenzaldehyde was treated as described in Key Intermediate 1, steps 4-6 to afford the title compound as a white solid.

### Example 266

### R)-1-[4-Chloro-3-(3-chloro-4-nitro-phenoxy)-2-fluoro-phenyl]-propylamine (Example 266A); and 3-{(R)-1-[4-Chloro-3-(3-chloro-4-nitro-phenoxy)-2-fluoro-phenyl]-propyl-amino}-butyramide (Example 266B); and (S)-3-{(R)-1-[3-(4-Amino-3-chloro-phenoxy)-4-chloro-2-fluoro-phenyl]-propyl-amino}-butyramide.dihydrochloride (Example 266C)

### Step 1

(R)-1-[4-Chloro-3-(3-chloro-4-nitro-phenoxy)-2-fluoro-phenyl]-propylamine (Example 266A—prepared as described in Example 112, steps 1 and 3 using 3-chloro-4-nitrophenyl boronic acid in step 1) (227 mg, 0.77 mmol) was reductively aminated with acetoacetamide (78 mg, 0.77 mmol) as described in Example 88 to give 3-{(R)-1-[3-(3-chloro-4-nitro-phenoxy)-4-chloro-2-fluoro-phenyl]-propyl-amino}-butyramide (Example 266B) (244 mg) as a mixture of diastereoisomers.

### Step 2

A mixture of iron (255 mg, 4.6 mmol), iron (II) sulphate heptahydrate (310 mg, 1.1 mmol) and 3-{(R)-1-[3-(3-chloro-4-nitro-phenoxy)-4-chloro-2-fluoro-phenyl]-propyl-amino}-butyramide (244 mg, 0.5 mmol) in dioxane (5 ml) and water (1 ml) was heated to reflux overnight. The reaction mixture was allowed to cool, then filtered. The filtrate was concentrated and purified by preparative hplc to generate the (R,S) isomer (Example 266C) (45 mg as a beige solid. Further elution provided the corresponding (R,R) isomer (Example 267) (100 mg) also as a beige solid.

### Example 271

### trans-N-(4-Chloro-2-fluoro-3-phenoxy-benzyl)-cyclohexane-1,4-diamine. dihydrochloride

### Step 1

1-Bromomethyl-4-chloro-2-fluoro-3-phenoxy-benzene was prepared by a method analogous to that of Key Intermediate 1, step 2. ¹H NMR (400 MHz, CDCl3): 7.37-7.22 (4H, m), 7.13-7.07 (1H, m), 6.92 (2H, d), 4.50 (2H, d).

### Step 2

A solution of 1-bromomethyl-4-chloro-2-fluoro-3-phenoxy-benzene (0.25 g, 0.792 mmol) in DMF (1.50 mL) was added to a solution of N-Boc-trans-1,4-cyclohexanediamine (0.204 g, 0.951 mmol) and pyridine (0.169 mL, 1.58 mmol) in DMF (1.25 mL) dropwise at 0° C. The mixture was left in the ice bath and was warmed to room temperature overnight. The reaction was diluted with Et₂O and water, the phases were separated and the aqueous layer was extracted into Et₂O (×3), combined organic extracts were dried (sodium sulfate), filtered and concentrated. The crude [4-(4-chloro-2-fluoro-3-phenoxy-benzylamino)-cyclohexyl]-carbamic acid tert-butyl ester was diluted with 1,4-dioxane (2.00 mL) and HCl (4M in 1,4-dioxane, 5.00 mL) was added and the mixture was left to stand for 5 hours before it was concentrated. Preparative HPLC followed by HCl salt formation provided [3-(4-amino-3-methyl-phenoxy)-2,4-difluoro-benzyl]-pyridin-4-yl-amine as the dihydrochloride salt, 102 mg.

### Example 272

### trans-N-(2-Fluoro-4-methyl-3-phenoxy-benzyl)-cyclohexane-1,4-diamine.dihydrochloride

To a microwave tube was added [4-(2-fluoro-4-methyl-3-phenoxy-benzylamino)-cyclohexyl]-carbamic acid tert-butyl ester (0.144 g, 0.321 mmol), methylboronic acid (0.0576 g, 0.962 mmol), palladium(II) acetate (0.00288 g, 0.0128 mmol), S-Phos (0.0105 g, 0.0257 mmol) and tripotassium phosphate (0.136 g, 0.641 mmol) followed by toluene (1.04 mL). The flask was evacuated and refilled with nitrogen twice before the tube was sealed and heated under microwave irradiation at 120° C. for 40 minutes. The mixture was then diluted with EtOAc, filtered, and concentrated. The crude material was diluted with EtOAc (2.00 mL) and HCl (saturated in EtOAc, 5.00 mL) was added and the mixture was left to stand for 5 hours before it was filtered and washed with EtOAc to give N-(2-fluoro-4-methyl-3-phenoxy-benzyl)-cyclohexane-1,4-diamine as the dihydrochloride salt, 95 mg.

### Example 273

### trans-N-(4-Chloro-2-fluoro-3-phenoxy-benzyl)-N-ethyl-cyclohexane-1,4-diamine.dihydrochloride

### Step 1

A solution of [4-(2-fluoro-4-methyl-3-phenoxy-benzylamino)-cyclohexyl]-carbamic acid tert-butyl ester (0.107 g, 0.238 mmol) in acetic anhydride (2.38 mL) and pyridine (2.38 mL) was stirred at room temperature overnight before it was concentrated. The residue was partitioned between water and CHCl₃ and extracted into CHCl₃ (×3). The combined organic extracts were dried (sodium sulfate), filtered and concentrated. The material was taken into EtOAc and saturated HCl in EtOAc was added dropwise. The reaction was stirred at room temperature overnight before the mixture was concentrated. Preparative HPLC provided N-(4-amino-cyclohexyl)-N-(4-chloro-2-fluoro-3-phenoxy-benzyl)-acetamide, 74 mg. ¹H NMR (Mixture of rotamers) (400 MHz, DMSO-d6): 7.99-7.72 (2H, m), 7.55-7.30 (3H, m), 7.22-7.02 (2H, m), 6.94-6.83 (2H, m), 4.59 (0.8H, s), 4.45 (1.2H, s), 4.30-4.16 (0.4H, m), 3.79-3.69 (0.6H, m), 2.99-2.87 (1H, m), 2.20 (1.6H, s), 1.98-1.87 (3.5H, m), 1.73 (1.2H, d), 1.63-1.31 (4.7H, m). MS: [M+Na]+413.0.

### Step 2

To a stirred solution of N-(4-amino-cyclohexyl)-N-(4-chloro-2-fluoro-3-phenoxy-benzyl)-acetamide (0.04 g, 0.102 mmol) in THF (0.256 mL) at 0° C. was added borane in THF (1M solution, 0.512 mL, 0.512 mmol) dropwise. The mixture was stirred at room temperature for overnight then at 50° C. for 5 hours before it was quenched at 0° C. by the addition of excess MeOH (˜3 mL). The mixture was stirred at room temperature for overnight before the solvents were removed under vacuum. Preparative HPLC provided N-(4-chloro-2-fluoro-3-phenoxy-benzyl)-N-ethyl-cyclohexane-1,4-diamine which was converted to the dihydrochloride salt, 9.1 mg.

### Example 274

### {3-[3-((S)-1-Amino-propyl)-6-chloro-2-fluoro-phenoxy]-5-fluoro-phenyl}-methanol.hydrochloride

### Step 1

Key Intermediate 3 (0.3 g, 0.975 mmol) was coupled with (3-fluoro-5-methoxycarbonyl-phenyl)boronic acid (0.297 g, 2.44 mmol) using the method described in Key Intermediate 1, step 1 providing 3-(6-chloro-3-{(S)-1-[(S)-2,2-dimethyl-propane-sulfinamide]-propyl}-2-fluoro-phenoxy)-5-fluoro-benzoic acid methyl ester. MS: [M+H]+ 460.0.

### Step 2

A solution of 3-(6-chloro-3-{(S)-1-[(S)-2,2-dimethyl-propanesulfinamide]-propyl}-2-fluoro-phenoxy)-5-fluoro-benzoic acid methyl ester (0.711 g, 1.55 mmol) and 1M lithium hydroxide (1M, 4.64 mL, 4.64 mmol) in 1,4-dioxane (7.73 mL) was stirred at room temperature for 3 hours before the solvents evaporated. The residue was partitioned between 5% citric acid solution and CHCl₃ and extracted into CHCl₃ (×3). The combined organic extracts were dried (sodium sulfate), filtered, concentrated and was used without further purification. To a stirred solution of 3-(6-chloro-3-{(S)-1-[(S)-2,2-dimethyl-propanesulfinamide]-propyl}-2-fluoro-phenoxy)-5-fluoro-benzoic acid in THF (3.82 mL) at 0° C. was added borane in THF (1M solution, 4.58 mL, 4.58 mmol) dropwise. The mixture was stirred at 50° C. for overnight before it was quenched at 0° C. by the addition of excess MeOH followed by piperazine (0.658 g, 7.64 mmol). The mixture was stirred at room temperature overnight before the solvents were removed under vacuum. The residue was taken into EtOAc, washed with water (×2), brine, dried (sodium sulfate), filtered, concentrated and was used without further purification. (S)—N-{(S)-1-[4-Chloro-2-fluoro-3-(3-fluoro-5-hydroxymethyl-phenoxy)-phenyl]propyl}-2,2-dimethyl-propanesulfinamide was taken into MeOH (3.09 mL), and 4M HCl in 1,4-dioxane (3.09 mL) was added dropwise. The reaction was stirred at room temperature for 1.5 hours before the mixture was concentrated (500 mg). 150 milligrams was subject to preparative HPLC and provided {3-[3-((S)-1-amino-propyl)-6-chloro-2-fluoro-phenoxy]-5-fluoro-phenyl}-methanol which was converted to the hydrochloride salt, 81 mg.

### Example 275

### [(S)-1-(4-Chloro-2-fluoro-3-phenoxy-phenyl)-propyl]-(1H-imidazol-2-yl)-amine.hydrochloride

To a microwave tube was added (S)-1-(2-chloro-4-fluoro-3-phenoxy-phenyl)-propylamine (0.2 g, 0.715 mmol) (prepared analogously to Key Intermediate 1), 2-chloroimidazole (0.088 g, 0.858 mmol), p-toluenesulfonic acid monohydrate (0.068 g, 0.357 mmol) and toluene (1.22 mL). The tube was evacuated and refilled with nitrogen twice before the tube was sealed and heated at 160° C. for 8 hours. Upon cooling the mixture was partitioned between CHCl₃ and saturated sodium hydrogen carbonate solution, the phases were separated and the aqueous layer was extracted into CHCl₃ (×3). Combined organic extracts were dried (sodium sulfate), filtered and concentrated. Preparative HPLC and provided [(S)-1-(4-chloro-2-fluoro-3-phenoxy-phenyl)-propyl]-(1H-imidazol-2-yl)-amine, 30.1 mg, and [(S)-1-(4-chloro-2-fluoro-3-phenoxy-phenyl)-propyl]-bis-(1H-imidazol-2-yl)-amine, 29.8 mg.

### Example 276

### (R)-1-(4-Chloro-2-fluoro-3-phenoxy-phenyl)-2-(tetrahydro-pyran-4-yl)-ethylamine. hydrochloride and (S)-1-(4-Chloro-2-fluoro-3-phenoxy-phenyl)-2-(tetrahydro-pyran-4-yl)-ethylamine.hydrochloride

### Step 1

A 2-neck flask fitted with a condenser and containing magnesium (0.397 g, 16.3 mmol) was made anhydrous by heating under a stream of N₂. The magnesium was stirred overnight before a small crystal of iodine and THF (24.5 mL) were added. 4-(Bromomethyl)-tetrahydropyran (2.66 g, 14.8 mmol) was added dropwise, over 30 minutes, whereupon the iodine colour paled significantly. Finally the mixture was heated at 50° C. for an additional 5 hours and then cooled to room temperature. To a stirred solution of (R)-2-methyl-propane-2-sulfinic acid 1-(4-chloro-2-fluoro-3-phenoxy-phenyl)-meth-(E)-ylideneamide (1.5 g, 4.24 mmol) in THF (29.7 mL) at −78° C. was added the Grignard solution dropwise. The mixture was left in the cold bath and allowed to warm to room temperature overnight before it was quenched at 0° C. by the addition of saturated ammonium chloride solution. The phases were separated and the aqueous phase was extracted into EtOAc (×3). Combined organic extracts were dried (sodium sulfate), filtered and concentrated. Column chromatography eluting with a gradient of 50% EtOAcpetrol to 100% EtOAc provided (R)—N—[(R)-1-(4-chloro-2-fluoro-3-phenoxy-phenyl)-2-(tetrahydro-pyran-4-yl)-ethyl]-2,2-dimethyl-propanesulfinamide, 432 mg. ¹H NMR (400 MHz, Me-d3-OD): 7.45-7.26 (4H, m), 7.08 (1H, t), 6.84 (2H, d), 4.76 (1H, t), 3.97-3.82 (2H, m), 3.45-3.34 (2H, m), 2.01-1.88 (1H, m), 1.83-1.71 (1H, m), 1.71-1.55 (3H, m), 1.41-1.25 (2H, m), 1.18 (9H, s). MS: [M+H]+ 454.0. Further elution with 2% MeOHEtOAc gave (R)—N—[(S)-1-(4-chloro-2-fluoro-3-phenoxy-phenyl)-2-(tetrahydro-pyran-4-yl)-ethyl]-2,2-dimethyl-propanesulfinamide, 1.25 g. ¹H NMR (400 MHz, Me-d3-OD): 7.47-7.26 (4H, m), 7.07 (1H, t), 6.93-6.82 (2H, m), 4.77-4.66 (1H, m), 3.94-3.87 (2H, m), 3.40-3.34 (2H, m), 2.00-1.88 (1H, m), 1.79-1.60 (4H, m), 1.37-1.27 (2H, m), 1.24 (9H, s). MS: [M+H]+ 454.0.

### Step 2

(R)-1-(4-Chloro-2-fluoro-3-phenoxy-phenyl)-2-(tetrahydro-pyran-4-yl)-ethylamine was prepared by a method analogous to that of Key Intermediate 1, step 6. (S)-1-(4-Chloro-2-fluoro-3-phenoxy-phenyl)-2-(tetrahydro-pyran-4-yl)-ethylamine was prepared by a method analogous to that of Key Intermediate 1, step 6.

### Example 277

### (S)—N-(2-Amino-ethyl)-3-[(R)-1-(4-chloro-2-fluoro-3-phenoxy-phenyl)-2-(tetrahydro-pyran-4-yl)-ethylamino]-butyramide.dihydrochloride

### Step 1

(R)-1-(4-Chloro-2-fluoro-3-phenoxy-phenyl)-2-(tetrahydro-pyran-4-yl)-ethylamine (0.425 g, 1.1 mmol) was converted to the free-base by partition between CHCl₃ and saturated sodium hydrogen carbonate solution, the phases were separated and the aqueous layer was extracted into CHCl₃ (×2). Combined organic extracts were dried (sodium sulfate), filtered and concentrated. To a reaction vial was added the (R)-1-(4-chloro-2-fluoro-3-phenoxy-phenyl)-2-(tetrahydro-pyran-4-yl)-ethylamine, lithium perchlorate (0.164 g, 1.54 mmol) and (2E)-1-[(3aS,6R,7aR)-tetrahydro-8,8-dimethyl-2,2-dioxido-3H-3a,6-methano-2,1-benzisothiazol-1(4H)-yl]-2-buten-1-one (0.374 g, 1.32 mmol). The tube was evacuated and refilled with nitrogen twice before the tube was stirred at room temperature for 5 days before the mixture was diluted with EtOAc, washed with water (×2), dried (sodium sulfate), filtered and concentrated. Column chromatography eluting with a gradient of 30% EtOAcpetrol to 60% EtOAcpetrol gave 3-[(R)-1-(4-chloro-2-fluoro-3-phenoxy-phenyl)-2-(tetrahydro-pyran-4-yl)-ethylamino]-1-[(3aS,6R,7aR)-tetrahydro-8,8-dimethyl-2,2-dioxido-3H-3a,6-methano-2,1-benzisothiazol-1(4H)-yl]-butan-1-one as a 3:1 mixture of diastereomers, 503 mg. MS: [M+H]+ 633.2.

### Step 2

A solution of 3-[(R)-1-(4-chloro-2-fluoro-3-phenoxy-phenyl)-2-(tetrahydro-pyran-4-yl)-ethylamino]-1-[(3aS,6R,7aR)-tetrahydro-8,8-dimethyl-2,2-dioxido-3H-3a,6-methano-2,1-benzisothiazol-1(4H)-yl]-butan-1-one (0.503 g, 0.794 mmol) in 1M lithium hydroxide (1M solution, 1.19 mL, 1.19 mmol) and THF (3.97 mL) was stirred at room temperature overnight before the solution was concentrated to dryness to give 3-[(R)-1-(4-chloro-2-fluoro-3-phenoxy-phenyl)-2-(tetrahydro-pyran-4-yl)-ethylamino]-butyric acid, 391 mg. Used without further purification. MS: [M+H]+ 436.0.

### Step 3

(S)—N-(2-Amino-ethyl)-3-[(R)-1-(4-chloro-2-fluoro-3-phenoxy-phenyl)-2-(tetrahydro-pyran-4-yl)-ethylamino]-butyramide and (R)—N-(2-amino-ethyl)-3-[(R)-1-(4-chloro-2-fluoro-3-phenoxy-phenyl)-2-(tetrahydro-pyran-4-yl)-ethylamino]-butyramide were prepared from 3-[(R)-1-(4-chloro-2-fluoro-3-phenoxy-phenyl)-2-(tetrahydro-pyran-4-yl)-ethylamino]-butyric acid (0.092 g, 0.211 mmol) by a method analogous to that of Example 223. (S)—N-(2-amino-ethyl)-3-[(R)-1-(4-chloro-2-fluoro-3-phenoxy-phenyl)-2-(tetrahydro-pyran-4-yl)-ethylamino]-butyramide, 14 mg: and (R)—N-(2-amino-ethyl)-3-[(R)-1-(4-chloro-2-fluoro-3-phenoxy-phenyl)-2-(tetrahydro-pyran-4-yl)-ethylamino]-butyramide, 9 mg:

### Example 279

### 1-[3-(4-Chloro-phenoxy)-2,4-difluoro-phenyl]-propylamine.hydrochloride

Chlorine gas was bubbled through a solution of 1-(2,4-difluoro-3-phenoxy-phenyl)-propylamine hydrochloride (100 mg) in 5% MeOHDCM (10 ml) for 5 minutes then the solution was stirred at room temperature overnight. The reaction mixture was diluted with DCM, washed with saturated sodium hydrogen carbonate solution, then dried over Na₂SO₄, filtered and evaporated. The residue was triturated with diethyl ether and the resultant solid collected by filtration and sucked dry to give 48 mg 1-[3-(4-chloro-phenoxy)-2,4-difluoro-phenyl]-propylamine as a white solid.

### Example 294

### 3-Amino-3-(2,4-difluoro-3-phenoxy-phenyl)-propan-1-ol.hydrochloride

To a stirred solution of 3-amino-3-(2,4-difluoro-3-phenoxy-phenyl)-propionic acid methyl ester (as described in Example 20) (0.136 g, 0.44 mmol) in THF (5 mL) at 0° C. was added lithium aluminium hydride in THF (2M solution, 0.66 mL, 1.3 mmol) dropwise. The mixture was stirred at room temperature for 1 h 30 mins, water (0.3 ml) was added carefully and then 1N NaOH (0.6 ml) and water (0.3 ml) were added successively. The resulting suspension was filtered through a plug of Na₂SO₄, evaporated under reduced pressure and the residue purified by flash column chromatography eluting with 2N NH₃ in MeOHDCM (3:97) to give 41 mg of 3-amino-3-(2,4-difluoro-3-phenoxy-phenyl)-propan-1-ol as a colourless powder.

### Example 314

### 3-{(R)-1-[(R)-1-(4-Chloro-2-fluoro-3-phenoxy-phenyl)-propylamino]-ethyl}-1H-pyridin-2-one.hydrochloride

To a suspension of (R)-1-(4-chloro-2-fluoro-3-phenoxy-phenyl)-propylamine hydrochloride (prepared in analogous manner to Key Intermediate 1, but using 6-chloro-2-fluoro-3-methyl phenol as starting material) (400 mg, 1.3 mmol) in DCE (6 ml) was added triethylamine (180 μl, 1.26 mmol), 1-(2-chloro-pyridin-3-yl)-ethanone (0.2 g, 1.26 mmol) and glacial acetic acid (156 μl, 2.6 mmol). The resulting mixture was stirred at room temperature for 24 h, and then for an additional 72 hr after sodium triacetoxyborohydride (540 mg, 2.6 mmol) was added. It was poured into 1 M sodium hydroxide and extracted into DCM and evaporated. The residue was heated under reflux for 48 h in a mixture 6 N HCl (3 ml) and THF (3 ml). The solvents were evaporated and the crude residue purified by preparative hplc to give the (R,R) isomer title compound (6 mg) as a white solid. Further elution afforded the (S,R) isomer (17 mg) as a white solid.

### Example 337

### (R)-1-(4-Chloro-2-fluoro-3-phenoxy-phenyl)-3-methoxy-propylamine hydrochloride

### Step 1

A solution di-tert-butyl dicarbonate (0.173 g, 0.8 mmol) in dioxane (2 ml) was added dropwise to a solution of (R)-3-amino-3-(4-chloro-2-fluoro-3-phenoxy-phenyl)-propan-1-ol hydrochloride (0.22 g, 0.7 mmol), prepared as described in Example 338, in dioxaneH₂O (3 ml4 ml) containing sodium hydrogen carbonate at 0° C. The reaction mixture was allowed to warm to room temperature and stirred over the weekend. Solvent evaporated, residue taken up in DCMH₂O, organic layer separated, dried over Na₂SO4, filtered and evaporated. Crude residue purified by flash column chromatography eluting with 5% MeOHDCM to give [(R)-1-(4-chloro-2-fluoro-3-phenoxy-phenyl)-3-hydroxy-propyl]-carbamic acid tert-butyl ester (0.24 g) as a colourless oil that solidifies upon standing. ¹H NMR (400 MHz, DMSO-d6): 7.48 (2H, d), 7.41-7.26 (4H, m), 7.18-7.05 (1H, m), 6.86 (3H, d), 4.94-4.79 (1H, m), 4.52 (1H, t), 3.57 (1H, s), 3.45-3.33 (2H, m), 1.90-1.78 (1H, m), 1.78-1.65 (1H, m), 1.36 (9H, s).

### Step 2

To a solution of [(R)-1-(4-chloro-2-fluoro-3-phenoxy-phenyl)-3-hydroxy-propyl]-carbamic acid tert-butyl ester (0.22 g, 0.55 mmol) in acetonitrile (5 ml) were added successively silver(I) oxide (1.3 g, 5.5 mmol) and methyl iodide (0.68 ml, 11 mmol). The reaction mixture was stirred for 48 h at room temperature, filtered through celite, filtrate evaporated and the residue purified by flash column chromatography eluting with 30% EtOAcPetrol to give [(R)-1-(4-chloro-2-fluoro-3-phenoxy-phenyl)-3-methoxy-propyl]-carbamic acid tert-butyl ester (0.175 g) as a colourless solid. ¹H NMR (400 MHz, DMSO-d6): 7.58-7.44 (2H, m), 7.41-7.28 (3H, m), 7.15-7.05 (1H, m), 6.86 (2H, d), 4.92-4.79 (1H, m), 3.39-3.33 (1H, m), 3.26-3.19 (1H, m), 3.17 (3H, s), 1.96-1.75 (2H, m), 1.40-1.16 (9H, m).

### Step 3

A solution of [(R)-1-(4-chloro-2-fluoro-3-phenoxy-phenyl)-3-methoxy-propyl]-carbamic acid tert-butyl ester (0.09 g, 0.22 mmol) was dissolved in EtOAc (3 ml) saturated with HCl and stirred for 1 h and the solvent was evaporated to dryness. The residue was triturated with Et₂O and the solid collected and dried to give (R)-1-(4-chloro-2-fluoro-3-phenoxy-phenyl)-3-methoxy-propylamine hydrochloride (58 mg) as a colourless powder.

### Example 338

### 3-Amino-3-(4-chloro-2-fluoro-3-phenoxy-phenyl)-propan-1-ol.hydrochloride

To a stirred solution of (S)-3-(4-chloro-2-fluoro-3-phenoxy-phenyl)-3-((R)-2-methyl-propane-2-sulfinylamino)-propionic acid (0.204 g, 0.49 mmol) (prepared as described for Key Intermediates 8 and 9 in THF (5 mL) at 0° C. was added borane in THF (1M solution, 1.2 mL, 1.2 mmol) dropwise. The mixture was stirred at room temperature for 1 h 30 mins, quenched by dropwise addition of 10% citric acid and extracted with DCM. The combined extract was washed with H₂O, dried over Na₂SO₄, filtered and evaporated. The residue was purified by flash column chromatography eluting with 5% MeOHDCM to give 0.16 g of (R)-2-Methyl-propane-2-sulfinic acid [(S)-1-(4-chloro-2-fluoro-3-phenoxy-phenyl)-3-hydroxy-propyl]-amide as a colourless powder. ¹H NMR (400 MHz, Me-d3-OD): 7.43-7.35 (2H, m), 7.35-7.26 (2H, m), 7.12-7.02 (1H, m), 6.84 (2H, d), 3.81-3.71 (1H, m), 3.70-3.60 (1H, m), 2.25-2.12 (1H, m), 2.09-1.97 (1H, m), 1.20 (9H, s). [M+H]⁺=400

To a stirred solution of (R)-2-methyl-propane-2-sulfinic acid [(S)-1-(4-chloro-2-fluoro-3-phenoxy-phenyl)-3-hydroxy-propyl]-amide (0.07 g, 0.17 mmol) in MeOH (2 ml) was added 4 N HCldioxane (00.3 ml). The mixture was stirred at room temperature for 1 h, the solvent was evaporated and the residue triturated with Et₂O and filtered to give 0.05 g of the title compound as a hydrochloride salt.

### Example 357

### (R)-1-(4-Chloro-2-fluoro-3-phenoxy-phenyl)-3-fluoro-propylamine.hydrochloride

To a solution of [(R)-1-(4-chloro-2-fluoro-3-phenoxy-phenyl)-3-hydroxy-propyl] carbamic acid tert-butyl ester (0.33 g, 0.83 mmol) (prepared as described in Example 337 step 1) in DCM at −78° C. under an inert atmosphere were successively added DBU (0.19 ml, 1.25 mmol) and XtalFluorE (0.29 g, 1.25 mmol) and the mixture stirred at −78° C. for 30 minutes and then allowed to warm to room temperature. The reaction was quenched with 5% aq. NaHCO₃, stirred for 15 minutes and extracted twice with DCM. Combined organics dried over Na₂SO₄, filtered, evaporated and the residue purified by flash column chromatography eluting with 50% to 100% EtOAc in petrol. The fractions with mass corresponding to the desired product were combined and evaporated. The residue was treated with 4N HCldioxane (3 ml) overnight and the solvent evaporated. Trituration of the resulting solid residue with Et₂O gave the title compound as a colourless powder (11 mg). 1H NMR (400 MHz, Me-d3-OD): 7.54 (1H, dd), 7.45 (1H, dd), 7.39-7.29 (2H, m), 7.16-7.06 (1H, m), 6.90 (2H, d), 4.80 (1H, dd), 4.74-4.64 (0.5H, m), 4.63-4.47 (1H, m), 4.46-4.36 (0.5H, m), 2.60-2.27 (2H, m). {M+H]+ 298.

### Example 361

### 4-[3-((R)-1-Amino-propyl)-6-chloro-2-fluoro-phenoxy]-phenylamine hydrochloride

(R)-2-Methyl-propane-2-sulfinic acid {(R)-1-[3-(4-amino-phenoxy)-4-chloro-2-fluoro-phenyl]-propyl}-amide (450 mg, 1.13 mmol, 1.0 eq) was dissolved in EtOAc (4.5 ml) and 2.1 M HCl in EtOAc (1.07 ml, 2.26 mmol, 2.0 eq) charged. After stirring for 1 hour MeOH (2 ml) and additional 2.1 M HCl in EtOAc (0.54 ml, 1.13 mmol, 1.0 eq) were added. After stirring for 30 minutes analysis (HPLC) indicated complete conversion and the mixture was concentrated in vacuo. The solid obtained was slurried in 3:1 hepaneEt₂O (16 ml), filtered off, washed with heptanes (3 ml) and was dried in vacuo at 30° C. overnight, to give 4-[3-((R)-1-amino-propyl)-6-chloro-2-fluoro-phenoxy]-phenylamine hydrochloride (377 mg, ¹H NMR >95%, 1.13 mmol, quantitative yield).

### Example 362

### (R)-1-[4-chloro-2-fluoro-3-(4-nitro-phenoxy)-phenyl]-propylamine hydrochloride

To a solution of (R)-2-methyl-propane-2-sulfinic acid {(R)-1-[4-chloro-2-fluoro-3-(4-nitro-phenoxy)-phenyl]-propyl}-amide (1.310 mg, 3.05 mmol, 1.0 eq) (Example 360 Step 1) in EtOAc (40 ml) was added 2.1 M HCl in EtOAc (4.5 ml, 9.5 mmol, 3.1 eq) and stirred at RT for 1 hour. The reaction was concentrated in vacuo and the residue was slurried in 3:1 heptane:Et₂O (30 ml) for 4 hours, the solids were filtered and washed with 3:1 heptane:Et₂O (2×10 ml). The solids were dried in an oven at 40° C. for overnight under vacuum to give (R)-1-[4-chloro-2-fluoro-3-(4-nitro-phenoxy)-phenyl]-propylamine hydrochloride (819 mg, ¹H NMR >95%, 2.27 mmol, 74% yield). ¹H NMR (270 MHz, DMSO-d₆): 8.83 (3H, s), 8.29-8.23 (2H, m), 7.78-7.67 (2H, m), 7.22-7.16 (2H, m), 4.38 (1H, q), 2.01-1.81 (2H, m), 0.83 (3H, t).

### Example 363

### N-{4-[3-((R)-1-Amino-propyl)-6-chloro-2-fluoro-phenoxy]-phenyl}-acetamide hydrochloride

### Step 1

To a solution of (R)-2-methyl-propane-2-sulfinic acid {(R)-1-[3-(4-amino-phenoxy)-4-chloro-2-fluoro-phenyl]-propyl}-amide (400 mg, 1.00 mmol, 1.0 eq) (Example 360 Step 2) in DCM (2 ml) was added neutral alumina (153 mg, 1.50 mmol, 1.5 eq) and acetic anhydride (0.09 ml, 1.10 mmol, 1.0 eq). After stirring for 1 hour analysis (HPLC) indicated complete conversion. The mixture was filtered then concentrated in vacuo, to give N-(4-{6-chloro-2-fluoro-3-[(R)-1-{(R)-2-methyl-propane-2-sulfinylamino}-propyl]-phenoxy}-phenyl)-acetamide (400 mg, ¹H NMR >95%, 0.91 mmol, 91% yield). ¹H NMR (270 MHz, CDCl₃): 7.40 (2H, d), 7.22-7.10 (3H, m), 6.84 (2H, d), 4.42 (1H, dd), 3.73 (1H, d), 2.06 (3H, s), 2.01-1.90 (1H, m), 1.86-1.74 (1H, m), 1.21 (9H, s), 0.88 (3H, t).

### Step 2

N-(4-{6-Chloro-2-fluoro-3-[(R)-1-{(R)-2-methyl-propane-2-sulfinylamino}-propyl]-phenoxy}-phenyl)-acetamide (450 mg, 0.91 mmol, 1.0 eq) was dissolved in EtOAc (4 ml) and 2.1 M HCl in EtOAc (0.9 ml, 1.80 mmol, 2.0 eq) charged. After stirring for 1 hour analysis (HPLC) indicated complete conversion and the mixture was concentrated in vacuo. The solid obtained was slurried in 3:1 hepaneEt₂O (15 ml), filtered off, washed with heptanes (3 ml) and was dried in vacuo at 30° C. overnight, to give N-{4-[3-((R)-1-Amino-propyl)-6-chloro-2-fluoro-phenoxy]-phenyl}-acetamide hydrochloride (270 mg, ¹H NMR >95% (excluding 7% solvents), 0.67 mmol, 74% yield).

### Example 364

### 3-{4-[3-((R)-1-Amino-propyl)-6-chloro-2-fluoro-phenoxy]-phenyl}-1,1-dimethyl-urea hydrochloride

### Step 1

To a solution of (R)-2-methyl-propane-2-sulfinic acid {(R)-1-[3-(4-amino-phenoxy)-4-chloro-2-fluoro-phenyl]-propyl}-amide (Example 360 Step 2) (400 mg, 1.00 mmol, 1.0 eq) in DCM (4 ml) was added Et₃N (0.42 ml, 3.00 mmol, 3.0 eq) and DMAP (5 mg) followed by dimethyl carbamoyl chloride (0.23 ml, 2.50 mmol, 2.5 eq) dropwise over 1 min. The mixture was heated to 40° C. and stirred for 3 days, after which time analysis (HPLC) indicated complete conversion. The mixture was cooled to room temperature, water (4 ml) added and the mixture stirred for 30 min. The layers were separated, the aqueous extracted with DCM (2×10 ml), the combined organics dried (MgSO₄), filtered and concentrated in vacuo. The residue was purified by column chromatography on silica (10 g), eluting with 1% MeOHEtOAc to give (R)-2-methyl-propane-2-sulfinic acid ((R)-1-{4-chloro-3-[4-(3,3-dimethyl-ureido)-phenoxy]-2-fluoro-phenyl}-propyl)-amide (410 mg, 1H NMR ˜90%, 0.79 mmol, 78% yield). 1H NMR (270 MHz, CDCl₃): 7.30-7.21 (3H, m), 7.14-7.09 (1H, m), 6.81 (2H, m), 6.23 (1H, br s), 4.44 (1H, dd), 3.52 (1H, d), 3.00 (6H, s), 1.99-1.91 (1H, m), 1.83-1.75 (1H, m), 1.20 (9H, s), 0.87 (3H, t).

### Step 2

(R)-2-Methyl-propane-2-sulfinic acid ((R)-1-{4-chloro-3-[4-(3,3-dimethyl-ureido)-phenoxy]-2-fluoro-phenyl}-propyl)-amide (400 mg (90%), 0.77 mmol, 1.0 eq) was dissolved in EtOAc (20 ml) and 2.1 M HCl in EtOAc (1.0 ml, 2.10 mmol, 2.75 eq) charged.

After stirring for 1 hour analysis (HPLC) indicated complete conversion and the suspension was filtered, the solid washed with Et₂O (3 ml) and dried in vacuo at 40° C. overnight, to give 3-{4-[3-((R)-1-Amino-propyl)-6-chloro-2-fluoro-phenoxy]-phenyl}-1,1-dimethyl-urea hydrochloride (225 mg, 1H NMR ˜95%, 0.56 mmol, 73% yield).

### Example 365

### 7-[3-((R)-1-Amino-propyl)-6-chloro-2-fluoro-phenoxy]-4H-benzo[1,4]oxazin-3-one hydrochloride

### Step 1

To a stirred mixture of 5-fluoro-2-nitro-phenol (10 g, 64 mmol, 1 eq) and K₂CO₃ (13.2 g, 96 mmol, 1.5 eq) in acetonitrile (360 ml) at 0° C. was added benzyl bromide (8.4 ml, 70 mmol, 1.1 eq) dropwise and the reaction was heated to 40° C. overnight. The reaction was cooled to room temperature, poured into water (350 ml), extracted with EtOAc (2×400 ml), washed with brine (400 ml), dried over MgSO₄, filtered and concentrated in vacuo. The crude material was purified via column chromatography (silica, 200 g) eluting with 5% EtOAc95% heptanes up to 20% EtOAc80% heptanes to give 2-benzyloxy-4-fluoro-1-nitro-benzene, (14.0 g, LC 97.2%, 56.6 mmol, 88% yield). 1H NMR (270 MHz, CDCl₃): 7.97 (1H, dd), 7.46-7.37 (5H, m), 6.83 (1H, dd), 6.77-6.71 (1H, m), 5.23 (2H, s).

### Step 2

To a flask was charged (R)-2-methyl-propane-2-sulfinic acid [(R)-1-(4-chloro-2-fluoro-3-hydroxy-phenyl)-propyl]-amide (5.22 g, 17.0 mmol, 1.0 eq), 2-benzyloxy-4-fluoro-1-nitro-benzene (5.03 g, 20.3 mmol, 1.2 eq), Cs₂CO₃ (11.04 g, 33.88 mmol, 2.0 eq) and DMSO (200 ml) and the stirred mixture was heated to 100° C. under N₂ overnight. The reaction was allowed to cool to RT, diluted with water (200 ml) and extracted with EtOAc (3×500 ml). The combined organics were washed with water (3×200 ml) and brine (3×100 ml), dried over MgSO₄, filtered and concentrated in vacuo. The residue was purified via column chromatography (silica, 470 g) eluting with 25% EtOAcheptanes to give (R)-2-methyl-propane-2-sulfinic acid {(R)-1-[3-(3-benzyloxy-4-nitro-phenoxy)-4-chloro-2-fluoro-phenyl]-propyl}-amide (8.1 g, ¹H NMR >80%, 80% active, 12.1 mmol, 71% yield). ¹H NMR (270 MHz, CDCl₃): 7.96 (1H, d), 7.50-7.12 (7H, m), 6.65 (1H, d), 6.55 (1H, dd), 5.19 (2H, m), 4.34 (1H, q), 3.53 (1H, d), 2.06-1.73 (2H, m), 1.18 (9H, s), 0.93 (3H, t).

### Step 3

To a solution of (R)-2-methyl-propane-2-sulfinic acid {(R)-1-[3-(3-benzyloxy-4-nitro-phenoxy)-4-chloro-2-fluoro-phenyl]-propyl}-amide (8.80 g, 16.4 mmol, 1.0 eq) in MeOH (400 ml) was added Fe powder (9.18 g, 164 mmol, 10.0 eq) and a solution of NH₄Cl (8.77 g, 164 mmol, 10.0 eq) in water (200 ml). The reaction was heated to 76° C. for 1 hour, cooled to RT, filtered through Celite and washed with MeOH (3×200 ml). The filtrate was concentrated in vacuo and extracted with DCM (2×150 ml). The organics were washed with brine (100 ml), phase separated and concentrated in vacuo. The crude material was adsorbed onto silica (38 g) and purified via column chromatography (silica, 430 g) eluting with 30% EtOAcheptanes to give (R)-2-methyl-propane-2-sulfinic acid {(R)-1-[3-(4-amino-3-benzyloxy-phenoxy)-4-chloro-2-fluoro-phenyl]-propyl}-amide (6.5 g, ¹H NMR >90%, 11.58 mmol, 71% yield). ¹H NMR (270 MHz, CDCl₃): 7.43-7.28 (5H, m), 7.22 (1H, dd), 7.12-7.06 (1H, m), 6.66-6.58 (2H, m), 6.30 (1H, dd), 5.03 (2H, m), 4.41 (1H, q), 3.62 (2H, s), 3.52 (1H, d), 2.05-1.78 (2H, m), 1.18 (9H, s), 0.88 (3H, t).

### Step 4

To a solution of (R)-2-methyl-propane-2-sulfinic acid {(R)-1-[3-(4-amino-3-benzyloxy-phenoxy)-4-chloro-2-fluoro-phenyl]-propyl}-amide (500 mg, 0.99 mmol, 1 eq) in DCM (11 ml) at 0° C. was added 1M BCl₃ in DCM (2 ml, 2 mmol, 2.0 eq) slowly. The reaction was stirred at 0° C. for 15 min. then warmed to RT and stirred for 1 hour. The reaction was poured onto ice (16 g) and stirred until >0° C. The organic layer was separated and the aqueous layer washed with Et₂O (2×30 ml). The aqueous layer was taken to pH 8 with NaHCO₃, extracted with DCM (2×30 ml), phase separated and concentrated in vacuo to give 300 mg of crude material (NMR indicated >85% debenzylated material). To a solution of the crude residue in THF (4.5 ml) was added a sat. aqueous solution of NaHCO₃ (7.5 ml) followed by chloroacetyl chloride (0.10 ml, 1.2 mmol) dropwise. The reaction was stirred at RT for 15 min then heated to 40° C. overnight. The reaction was heated up to 60° C. for a further 5.5 hours then cooled to RT, dissolved in EtOAc (20 ml), separated and the organics washed with brine (15 ml), dried over MgSO₄, filtered and concentrated in vacuo. The crude material was purified via column chromatography (silica, 15 g) eluting with 1:1 EtOAc:heptanes to afford (R)-2-methyl-propane-2-sulfinic acid {(R)-1-[4-chloro-2-fluoro-3-(3-oxo-3,4-dihydro-2H-benzo[1,4]oxazin-7-yloxy)-phenyl]-propyl}-amide (200 mg, ¹H NMR >80%, 0.352 mmol, 36% yield). 1H NMR (270 MHz, CDCl₃): 9.43 (1H, s), 7.20-7.08 (2H, m), 6.63-6.59 (2H, m), 6.28 (1H, dd), 4.58-4.38 (4H, m), 2.04-1.71 (2H, m), 1.23 (9H, s), 0.89 (3H, t).

### Step 5

To a solution of (R)-2-methyl-propane-2-sulfinic acid {(R)-1-[4-chloro-2-fluoro-3-(3-oxo-3,4-dihydro-2H-benzo[1,4]oxazin-7-yloxy)-phenyl]-propyl}-amide (100 mg, 0.220 mmol, 1.0 eq) in EtOAc (20 ml) was added 2.1M HCl in EtOAc (0.31 ml, 0.651 mmol, 3.0 eq) slowly. The suspension was stirred at RT for 30 min. then concentrated in vacuo. The residue was slurried in 1:3 Et₂O:heptanes (12 ml) for 1 hour, filtered and washed with heptanes (5 ml) to give 7-[3-((R)-1-amino-propyl)-6-chloro-2-fluoro-phenoxy]-4H-benzo[1,4]oxazin-3-one hydrochloride (60 mg, ¹H NMR >95%, 0.155 mmol, 70% yield).

### Example 366

### (R)-1-[4-Chloro-3-(3,4-dihydro-2H-benzo[1,4]oxazin-7-yloxy)-2-fluoro-phenyl]-propylamine dihydrochloride

### Step 1

To a solution of (R)-2-methyl-propane-2-sulfinic acid {(R)-1-[4-chloro-2-fluoro-3-(3-oxo-3,4-dihydro-2H-benzo[1,4]oxazin-7-yloxy)-phenyl]-propyl}-amide (85 mg, 0.187 mmol, 1.0 eq) (Example 365 Step 4) in EtOAc (15 ml) was added 2.1M HCl in EtOAc (3 ml, 6.3 mmol, 33.7 eq) slowly. The suspension was stirred at RT for 1 hour then concentrated in vacuo. The residue was slurried in 1:3 Et₂O:heptanes (10 ml) for 1 hour, filtered and washed with heptanes (5 ml). To a solution of the collected solids in DCM (10 ml) was added sat. aqueous NaHCO₃ (5 ml) and the mixture was stirred until all the solids were dissolved. The layers were separated and the aqueous layer was washed with DCM (10 ml). The collected organic extracts were passed through a phase separator and concentrated in vacuo. The residue was dissolved in THF (0.16 ml) and cooled to 0° C. To the stirred solution was added 1M borane.THF complex in THF (0.26 ml 0.26 mmol, 1.4 eq) dropwise over 1 min. The reaction was warmed to RT and stirred overnight. Additional 1M borane.THF complex in THF (0.10 ml, 0.10 mmol, 0.5 eq) was added and the reaction was stirred for 1 hour. To the reaction MeOH (1 ml) was added dropwise and then stirred for 1 hour at RT then concentrated in vacuo. The residue was dissolved in MeOH (0.5 ml), 4M HCl in dioxane (0.2 ml, 0.8 mmol, 4.3 eq) was added and the reaction stirred for 15 min then concentrated in vacuo. The residue was partitioned between sat. aq. NaHCO₃ (1 ml) and DCM (2 ml) and the organic layer separated and concentrated in vacuo. The residue was purified via chromatography (silica, 10 g) eluting with 10% MeOHDCM. The oil was triturated with Et₂O (3 ml) and the solid was dissolved in EtOAc (0.3 ml) and 2.1M HCl in EtOAc (0.5 ml, 1.05 mmol, 5.6 eq). The mixture was concentrated in vacuo and the solid was dried in an oven at 30° C. overnight under vacuum to give (R)-1-[4-chloro-3-(3,4-dihydro-2H-benzo[1,4]oxazin-7-yloxy)-2-fluoro-phenyl]-propylamine hydrochloride (34 mg, ¹H NMR 80%, 80% active, 0.073 mmol, 39% yield).

### Example 367

### (R)-1-[4-Chloro-3-(2,3-dihydro-benzo[1,4]dioxin-6-yloxy)-2-fluoro-phenyl]-propylamine hydrochloride

### Step 1

To a solution of [(R)-1-(4-chloro-2-fluoro-3-hydroxy-phenyl)-propyl]-carbamic acid tert-butyl ester (470 mg, 1.55 mmol, 1.0 eq) in DCM (75 ml) was added 3,4-(ethylenedioxy)benzene boronic acid (555 mg, 3.09 mmol, 2.0 eq) and powdered 4 A molecular sieves (380 mg), followed by pyridine (0.30 ml, 3.71 mmol, 2.4 eq) and the mixture stirred until the majority was in solution. Cu(OAc)₂ (367 mg, 2.02 mmol, 1.3 eq) was added and the mixture stirred under air for 3 days, after which time analysis (LC) indicated approximately 30% product formation. The mixture was diluted with water (75 ml), stirred for 30 minutes, then the layers separated and the aqueous extracted with DCM (2×50 ml). The combined organics were dried (MgSO₄), filtered and concentrated in vacuo. The crude material was purified by chromatography on silica (50 g) eluting with DCM to provide {(R)-1-[4-chloro-3-(2,3-dihydro-benzo[1,4]dioxin-6-yloxy)-2-fluoro-phenyl]-propyl}-carbamic acid tert-butyl ester (182 mg, ¹H NMR >95%, 0.42 mmol, 26.8% yield). ¹H NMR (270 MHz, CDCl₃): 7.20-7.18 (1H, m), 7.09-7.01 (1H, m), 6.78-6.73 (1H, m), 6.40-6.36 (2H, m), 4.94 (1H, br s), 4.75-4.63 (1H, m), 4.27-4.16 (4H, m), 1.81-1.68 (2H, m), 1.40 (9H, br s), 0.89 (3H, t).

### Step 2

{(R)-1-[4-Chloro-3-(2,3-dihydro-benzo[1,4]dioxin-6-yloxy)-2-fluoro-phenyl]-propyl}-carbamic acid tert-butyl ester (200 mg, 0.46 mmol, 1.0 eq) was dissolved in EtOAc (0.5 ml) and 2.1 M HCl in EtOAc (1.0 ml, 2.10 mmol, 4.6 eq) charged. After stirring for 1 hour analysis (HPLC) indicated 30% conversion, therefore 4 M HCl in EtOAc (0.5 ml, 2.00 mmol) was added and the mixture stirred overnight. Analysis (HPLC) indicated complete conversion after this time therefore the mixture was concentrated in vacuo, followed by heptanes azeotrope. The resulting solid was dried in vacuo at 40° C. overnight, to give 150 mg (R)-1-[4-chloro-3-(2,3-dihydro-benzo[1,4]dioxin-6-yloxy)-2-fluoro-phenyl]-propylamine hydrochloride (150 mg, ¹H NMR >95%, 0.40 mmol, 87% yield).

### Example 368

### (R)-1-[4-Chloro-2-fluoro-3-(pyridin-4-yloxy)-phenyl]-propylamine hydrochloride

### Step 1

To a solution of Key Intermediate KI-3a, (R)-2-methyl-propane-2-sulfinic acid [(R)-1-(4-chloro-2-fluoro-3-hydroxy-phenyl)-propyl]-amide (1.0 g, 3.25 mmol, 1.0 eq) in DMA (20 ml) was added potassium tert-butoxide (365 mg, 3.25 mol, 1.0 eq). The solution was stirred for 1 hour to give a yellow solution before addition of 2-chloro-4-fluoropyridine (855 mg, 6.50 mmol, 2.0 eq). The reaction was held at 100° C. for 16 hours and then allowed to cool to room temperature. Water (100 ml) was added and extracted with DCM (2×30 ml). The organic layers were washed with 10% aq. K₂CO₃ solution (30 ml), water (30 ml) and sat. brine (30 ml). The solution was dried, filtered and concentrated directly onto silica (2 g). The material was purified by column chromatography on silica (50 g), eluting with 2:1 up to 1:1 heptanesEtOAc. The product fractions were combined and concentrated to give (R)-2-methyl-propane-2-sulfinic acid {(R)-1-[4-chloro-3-(2-chloro-pyridin-4-yloxy)-2-fluoro-phenyl]-propyl}-amide (800 mg, ¹H NMR >95% excluding solvent, 50% active, 0.95 mmol, 29% yield). ¹H NMR (270 MHz, CDCl₃): 8.26 (1H, d), 7.32-7.15 (2H, m), 6.89 (1H, d), 6.80 (1H, dd), 4.40 (1H, q), 3.53 (1H, d), 2.10-1.50 (2H, m), 1.45 (9H, s), 0.87 (3H, t).

### Step 2

To a solution of (R)-2-methyl-propane-2-sulfinic acid {(R)-1-[4-chloro-3-(2-chloro-pyridin-4-yloxy)-2-fluoro-phenyl]-propyl}-amide (1.10 g, 2.38 mmol, 1.0 eq) in MeOH (30 ml) was added ammonium formate (826 mg, 13.1 mmol, 5.5 eq) and 10% PdC (50% wet, 0.1 g). The mixture was heated at reflux for 2 hours. Additional ammonium formate (826 mg, 13.1 mmol, 5.5 eq) and 10% PdC (50% wet, 0.1 g) were added and the mixture heated at reflux for 4 hours. The catalyst was filtered off and fresh 10% PdC (50% wet, 0.1 g) added. After an additional reflux for 6 hours the catalyst was filtered off and washed with MeOH (10 ml). The solvent was removed in vacuo and the residue extracted into DCM (40 ml) and concentrated to give 901 mg of a crude yellow oil. The material was adsorbed onto silica (2 g) and purified by column chromatography on silica (30 g), eluting with 1:1 EtOAcheptanes up to 100% EtOAc. The product fractions were combined and concentrated to give (R)-2-methyl-propane-2-sulfinic acid {(R)-1-[4-chloro-2-fluoro-3-(pyridin-4-yloxy)-phenyl]-propyl}-amide (301 mg, 1H NMR >90%, 0.70 mmol, 29% yield). 1H NMR (270 MHz, CDCl₃): 8.49 (2H, d), 7.30-7.15 (2H, m), 6.83 (2H, d), 4.42 (1H, q), 3.56 (1H, d), 2.10-1.75 (2H, m), 1.21 (9H, s), 0.91 (3H, t).

### Step 3

(R)-2-methyl-propane-2-sulfinic acid {(R)-1-[4-chloro-2-fluoro-3-(pyridin-4-yloxy)-phenyl]-propyl}-amide (301 mg, 0.78 mmol) was dissolved in EtOAc (8 ml) and 2.1 M HCl in EtOAc (1.5 ml, 3.15 mmol) charged. After stirring for 1 hour, the solid was filtered off and washed with EtOAc (2 ml). The material was dried to give (R)-1-[4-chloro-2-fluoro-3-(pyridin-4-yloxy)-phenyl]-propylamine hydrochloride (175 mg, 0.55 mmol, 71% yield)—see table 2.

### Example 369

### (R)-1-[4-Chloro-2-fluoro-3-(pyridin-2-yloxy)-phenyl]-propylamine hydrochloride

### Step 1

A solution of 2-bromopyridine (360 mg, 2.28 mmol, 1.0 eq) and 2-pyridyl acetone (62 mg, 0.48 mmol, 0.2 eq) in N-methyl-2-pyrrolidone (14 ml) was vacuum degassed three times (release to nitrogen). Key Intermediate KI-3a, (R)-2-methyl-propane-2-sulfinic acid [(R)-1-(4-chloro-2-fluoro-3-hydroxy-phenyl)-propyl]-amide, (700 mg, 2.28 mmol, 1.0 eq) was added, followed by Cs₂CO₃ (1.48 g, 4.56 mmol, 2.0 eq) and CuBr (164 mg, 1.14 mmol, 0.5 eq), with further vacuum degassing performed after each addition. Once all reagents were added the mixture was heated to 115° C. and stirred for 16 hours after which time analysis (HPLC) showed 69% product and 21% starting material. The mixture was cooled to room temperature then poured into water (150 ml), the resulting suspension filtered, the solid washed with water and sucked dry. The solid was partitioned between water (50 ml) and DCM (50 ml), the mixture filtered and the filtrate layers separated. The aqueous was extracted with DCM (50 ml) then the combined organics passed through a phase separator and concentrated in vacuo. The crude material was purified by column chromatography on silica (10 g), eluting with DCM to 1% MeOHDCM. The product fractions were combined and concentrated to give (R)-2-methyl-propane-2-sulfinic acid {(R)-1-[4-chloro-2-fluoro-3-(pyridin-2-yloxy)-phenyl]-propyl}-amide (483 mg, 1H NMR >95%, 1.25 mmol, 55% yield). ¹H NMR (270 MHz, CDCl₃): 8.08 (1H, dd), 7.75-7.68 (1H, m), 7.27-7.24 (1H, m), 7.17 (1H, d), 7.08-6.98 (2H, m), 4.57 (1H, dd), 3.51 (1H, d), 2.04-1.94 (1H, m), 1.87-1.73 (1H, m), 1.21 (9H, s), 0.87 (3H, t).

### Step 2

(R)-2-Methyl-propane-2-sulfinic acid {(R)-1-[4-chloro-2-fluoro-3-(pyridin-2-yloxy)-phenyl]-propyl}-amide (420 mg, 1.09 mmol, 1.0 eq) was dissolved in EtOAc (25 ml) and 2 M HCl in EtOAc (1.04 ml, 2.18 mmol, 2.0 eq) charged. After stirring for 1 hour, the mixture was concentrated in vacuo. The solid was slurried in 3:1 heptaneEt₂O (12 ml), filtered off and washed with heptanes (3 ml). The material was dried to give (R)-1-[4-chloro-2-fluoro-3-(pyridin-2-yloxy)-phenyl]-propylamine hydrochloride (325 mg, ¹H NMR >95%, 1.02 mmol, 94% yield).

### Example 370

### 4-[3-((R)-1-Amino-propyl)-6-chloro-2-fluoro-phenoxy]-pyridin-2-ylamine hydrochloride

### Step 1

To a solution of the compound of Example 368 Step 1, (R)-2-methyl-propane-2-sulfinic acid {(R)-1-[4-chloro-3-(2-chloro-pyridin-4-yloxy)-2-fluoro-phenyl]-propyl}-amide, (2.6 g, 6.20 mmol, 1.0 eq) in EtOAc (40 ml) was added 2M HCl in EtOAc (10 ml, 20 mmol). After 16 hours at RT, the solvent was removed in vacuo and the residue azeotroped with toluene (500 ml). The solids were slurried in Et₂O (50 ml) and filtered to give 2.8 g of the HCl salt. This was added to DCM (15 ml) followed by sat. NaHCO₃ (10 ml). The organic layer was separated off and concentrated in vacuo to give 1520 mg (4.83 mmol) of the amine. This was redissolved in THF (25 ml) before charging sat. NaHCO₃ solution (25 ml) and di-tert-butyl dicarbonate (1105 mg, 5.06 mmol). After 16 hours, EtOAc (20 ml) was added and the organic layer separated off, washed with brine (10 ml), before being dried (MgSO₄), filtered and concentrated to give 1.96 g crude solid. The material was adsorbed onto silica (5 g) and purified by column chromatography on silica (60 g), eluting with 1:3 EtOAcheptanes. The product fractions were combined and concentrated to give {(R)-1-[4-chloro-3-(2-chloro-pyridin-4-yloxy)-2-fluoro-phenyl]-propyl}-carbamic acid tert-butyl ester (1.51 g, ¹H NMR >95% excluding solvent, 88% active, 3.20 mmol, 52% yield). ¹H NMR (270 MHz, CDCl₃): 8.26 (1H, d), 7.30-7.10 (2H, m), 6.75 (1H, m), 4.91 (1, bs), 4.71 (1H, obs bq), 1.70-1.65 (2H, m), 1.37 (9H, s), 0.91 (3H, t).

### Step 2

To {(R)-1-[4-chloro-3-(2-chloro-pyridin-4-yloxy)-2-fluoro-phenyl]-propyl}-carbamic acid tert-butyl ester (1400 mg, 3.37 mmol, 1.0 eq) was added palladium (II) chloride (30 mg, 0.17 mmol, 5 mol %), 1,8-diazabicycloundec-7-ene (560 mg, 3.68 mmol, 1.1 eq), 1,3-bis(diphenylphosphino)propane (140 mg, 0.34 mmol, 10 mol %) and 1-butanol (40 ml). Carbon monoxide gas (1 Lmin) was passed through the reaction whilst warming to 100° C. After 3.5 hours at 100° C., the reaction was cooled and EtOAc (30 ml) charged before filtering through Celite (10 g). The solvent was removed in vacuo, toluene (30 ml) charged and the solvent was removed in vacuo. The crude material was adsorbed onto silica (3 g) and purified by column chromatography on silica (40 g), eluting with 1:3 EtOAcheptanes. The product fractions were combined and concentrated to give 4-[3-((R)-1-tert-butoxycarbonylamino-propyl)-6-chloro-2-fluoro-phenoxy]-pyridine-2-carboxylic acid butyl ester (1530 mg, ¹H NMR >95% excluding solvent, 89% active, 2.83 mmol, 84% yield). 1H NMR (270 MHz, CDCl₃): 8.60 (1H, d), 7.64 (1H, d), 7.30-7.10 (2H, m), 6.90 (1H, dd), 4.92 (1H, bs), 4.73 (1H, m), 4.39 (2H, t), 1.85-1.70 (4H, m), 1.40 (2H, m) 1.37 (9H, s), 0.95 (3H, t), 0.91 (3H, t).

### Step 3

To 4-[3-((R)-1-tert-butoxycarbonylamino-propyl)-6-chloro-2-fluoro-phenoxy]-pyridine-2-carboxylic acid butyl ester (1350 mg, 2.81 mmol, 1.0 eq) in THF (20 ml) was added 1M aqueous LiOH (20 ml, 20 mmol, 7.1 eq). After 3 hours, the THF was removed in vacuo and the aqueous layer washed with Et₂O (2×10 ml), before being acidified to pH 4 by the addition of 10% citric acid (10 ml). Extraction with EtOAc (2×20 ml) and concentration gave 4-[3-((R)-1-tert-butoxycarbonylamino-propyl)-6-chloro-2-fluoro-phenoxy]-pyridine-2-carboxylic acid (1100 mg, ¹H NMR >95% excluding solvent, 93% active, 2.41 mmol, 86% yield). 1H NMR (270 MHz, CDCl₃): 13.0-12.0 (1H, bs), 8.63 (1H, d), 7.70-7.20 (4H, m), 4.64 (1H, q), 3.33 (1H, d), 1.80-1.50 (2H, m), 1.36 (9H, s), 0.83 (3H, t).

### Step 4

A mixture of 4-[3-((R)-1-tert-butoxycarbonylamino-propyl)-6-chloro-2-fluoro-phenoxy]-pyridine-2-carboxylic acid (700 mg, 1.65 mmol, 1.0 eq), diphenylphosphoryl azide (650 mg, 2.36 mmol, 1.43 eq) and triethylamine (245 mg, 2.42 mmol, 1.47 eq) in DMF (18 ml) was stirred for 16 hours at ambient temperature. Water (2 ml, 111 mmol, 67.3 eq) was added and the reaction heated at 100° C. for 2 hours. The reaction was concentrated before addition of EtOAc (30 ml). The organic layer was washed with water (30 ml), 10% LiCl (30 ml) and sat. brine (30 ml) before undergoing drying and concentration in vacuo. The material was dissolved in DCM and loaded onto a SCX-2 (10 g) column. This was eluted with 100% DCM then 100% MeOH then 100% 7N NH₃ in MeOH. The product fractions were concentrated and further purified by column chromatography on silica (10 g), eluting with 1:1 EtOAcheptanes. The product fractions were combined and concentrated to give {(R)-1-[3-(2-amino-pyridin-4-yloxy)-4-chloro-2-fluoro-phenyl]-propyl}-carbamic acid tert-butyl ester (141 mg, ¹H NMR >90%, 0.36 mmol, 22% yield). 1H NMR (270 MHz, CDCl₃): 7.93 (1H, d), 7.30-7.05 (2H, m), 6.21 (1H, d), 5.88 (1H, d), 5.00 (1H, d), 4.74 (1H, q), 4.49 (2H, bs), 1.80-1.50 (2H, m), 1.40 (9H, s), 0.75 (3H, t).

### Step 5

To {(R)-1-[3-(2-amino-pyridin-4-yloxy)-4-chloro-2-fluoro-phenyl]-propyl}-carbamic acid tert-butyl ester (70 mg, 0.177 mmol, 1.0 eq) in EtOAc (0.6 ml) was added 4M HCl in EtOAc (1.4 ml). After 20 hours at ambient temperature, additional 4M HCl in EtOAc (0.5 ml) was added. After 1 hour, the reaction was filtered and the solids washed with Et₂O (3 ml). The solid was oven dried at 40° C. to give 4-[3-((R)-1-amino-propyl)-6-chloro-2-fluoro-phenoxy]-pyridin-2-ylamine hydrochloride (39 mg, 0.117 mmol, 66% yield)—see table 2.

### Example 371

### N-{4-[3-((R)-1-Amino-propyl)-6-chloro-2-fluoro-phenoxy]-pyridin-2-yl}-acetamide hydrochloride

### Step 1

To the compound of Example 370 Step 4, {(R)-1-[3-(2-amino-pyridin-4-yloxy)-4-chloro-2-fluoro-phenyl]-propyl}-carbamic acid tert-butyl ester, (70 mg, 0.177 mmol, 1.0 eq) in DCM (5 ml) was added acetic anhydride (20 mg, 0.195 mmol, 1.10 eq) and pyridine (20 μl, 0.248 mmol, 1.4 eq). After 16 hours, the reaction was washed with sat. NaHCO₃ solution (3 ml) and sat. brine (3 ml). After concentration in vacuo, the material was purified by column chromatography on silica (1 g), eluting with 1:1 EtOAcheptanes. The product fractions were combined and concentrated to give {(R)-1-[3-(2-acetylamino-pyridin-4-yloxy)-4-chloro-2-fluoro-phenyl]-propyl}-carbamic acid tert-butyl ester (91 mg, ¹H NMR >90% excluding solvents, 85% active, 0.177 mmol, 100% yield). ¹H NMR (270 MHz, CDCl₃): 8.91 (1H, bs), 8.05 (1H, d), 7.84 (1H, obs s), 7.23 (1H, d), 7.13 (1H, t), 6.52 (1H, dd), 4.96 (1H, obs bs), 4.72 (1H, obs bs), 2.16 (3H, s), 1.80-1.50 (2H, m), 1.40 (9H, s), 0.91 (3H, t).

### Step 2

{(R)-1-[3-(2-Acetylamino-pyridin-4-yloxy)-4-chloro-2-fluoro-phenyl]-propyl}-carbamic acid tert-butyl ester (77 mg, 0.177 mmol, 1.0 eq) was dissolved in EtOAc (0.7 ml) and 4M HCl in EtOAc (1.5 ml) added. After 2 hours, the reaction was concentrated in vacuo. The solid was slurried in Et₂O (2 ml), filtered, washed with Et₂O (2 ml) and dried to give N-{4-[3-((R)-1-amino-propyl)-6-chloro-2-fluoro-phenoxy]-pyridin-2-yl}-acetamide hydrochloride (23 mg, 0.061 mmol, 35% yield).

### Example 372

### (R)-1-{4-Chloro-2-fluoro-3-[4-(2H-pyrazol-3-yl)-phenoxy]-phenyl}-propylamine hydrochloride

### Step 1

To a solution of Key Intermediate KI-3e, [(R)-1-(4-chloro-2-fluoro-3-hydroxy-phenyl)-propyl]-carbamic acid tert-butyl ester, (2.0 g, 6.59 mmol, 1.0 eq) in DCM (320 ml) was added 4-acetylphenyl boronic acid (2.16 g, 13.17 mmol, 2.0 eq) and powdered 4 A molecular sieves (1.6 g), followed by pyridine (1.32 ml, 15.37 mmol, 2.3 eq) and the mixture stirred until the majority was in solution. Cu(OAc)₂ (1.56 g, 8.59 mmol, 1.3 eq) was added and the mixture stirred under air for 3 days, after which time analysis (LC) indicated approximately 30% product formation. The mixture was diluted with water (320 ml), stirred for 30 minutes, then the layers separated and the aqueous extracted with DCM (200 ml). The combined organics were dried (MgSO₄), filtered and concentrated in vacuo. The crude material was purified by chromatography on silica (250 g) eluting with DCM to provide {(R)-1-[3-(4-acetyl-phenoxy)-4-chloro-2-fluoro-phenyl]-propyl}-carbamic acid tert-butyl ester (1.2 g, 1H NMR ˜70%, 1.99 mmol, 30.2% yield). 1H NMR (270 MHz, CDCl₃): 8.00-7.92 (2H, m), 7.27-7.23 (1H, m), 7.15-7.06 (1H, m), 6.89 (2H, m), 4.93 (1H, br s), 4.73 (1H, m), 2.56 (3H, s), 1.81-1.71 (2H, m), 1.41 (9H, br s), 0.91 (3H, t).

### Step 2

To a solution of {(R)-1-[3-(4-acetyl-phenoxy)-4-chloro-2-fluoro-phenyl]-propyl}-carbamic acid tert-butyl ester (710 mg, 70% purity, 1.18 mmol, 1.0 eq) in toluene (1.5 ml) was added DMF.DMA (0.57 ml, 4.29 mmol, 3.6 eq) and the mixture heated to 110° C. and stirred for 2 days with further DMF.DMA added over this time (2×0.6 ml). After this time analysis (HPLC) showed 45% product (4% starting material). The mixture was cooled to rt, concentrated in vacuo and the residue azeotroped with toluene (10 ml). The residue was dissolved in EtOH (15 ml), NH₂NH₂.H₂O (0.125 ml, 2.5 mmol, 2.2 eq) added, the mixture heated to reflux and stirred for 1 hour. After this time analysis (HPLC) indicated complete conversion to product. The mixture was cooled to room temperature, diluted with EtOAc (50 ml), washed with water (3×15 ml), dried (MgSO₄), filtered and concentrated in vacuo. The residue was suspended in heptaneEt₂O (31, 80 ml), heated to reflux, allowed to cool to room temperature and the resulting solid filtered off. The filtrate was concentrated and the residue purified by column chromatography on silica (100 g) eluting with 30% EtOAc in heptanes to give ((R)-1-{4-chloro-2-fluoro-3-[4-(2H-pyrazol-3-yl)-phenoxy]-phenyl}-propyl)-carbamic acid tert-butyl ester (350 mg, ¹H NMR ˜95%, 0.78 mmol, 66% yield). 1H NMR (270 MHz, CDCl₃): 7.68 (2H, m), 7.58 (1H, d), 7.25-7.20 (1H, m), 7.14-7.06 (1H, m), 6.96-6.86 (2H, m), 6.54 (1H, d), 4.93 (1H, br s), 4.69 (1H, m), 1.82-1.72 (2H, m), 1.40 (9H, br s), 0.90 (3H, t).

### Step 3

((R)-1-{4-Chloro-2-fluoro-3-[4-(2H-pyrazol-3-yl)-phenoxy]-phenyl}-propyl)-carbamic acid tert-butyl ester (429 mg, 0.96 mmol, 1.0 eq) was dissolved in EtOAc (5 ml) and 4 M HCl in EtOAc (15 ml) charged. After stirring for 5 hour, the mixture was concentrated in vacuo. The solid was slurried in Et₂O (5 ml), filtered off and washed with Et₂O (2 ml). The material was dried to give (R)-1-{4-Chloro-2-fluoro-3-[4-(2H-pyrazol-3-yl)-phenoxy]-phenyl}-propylamine hydrochloride (266 mg, 1H NMR >95%, 0.77 mmol, 73% yield).

### Example 373

### 5-[3-((R)-1-Amino-propyl)-6-chloro-2-fluoro-phenoxy]-2-fluoro-benzamide hydrochloride

### Step 1

To a solution of Key Intermediate KI-3e, [(R)-1-(4-chloro-2-fluoro-3-hydroxy-phenyl)-propyl]-carbamic acid tert-butyl ester, (10.0 g, 33.0 mmol, 1.0 eq) in DCM (1.6 L) was added methyl 3-carboxy 4-fluorophenyl boronic acid (12.6 g, 66.0 mmol, 2.0 eq) and powdered 4 A molecular sieves (8 g), followed by pyridine (6.6 ml, 81.6 mmol, 2.5 eq) and the mixture stirred until the majority was in solution. Cu(OAc)₂ (7.8 g, 42.9 mmol, 1.3 eq) was added and the mixture stirred under air for 2 days, after which time analysis (LC) indicated approximately 25% product formation. The mixture was diluted with water (1.6 L), stirred for 30 minutes, filtered then the layers separated and the aqueous extracted with DCM (2×400 ml). The combined organics were dried (MgSO₄), filtered and concentrated in vacuo. The crude material was purified by chromatography on silica (1 kg) eluting with DCM to provide 5-[3-((R)-1-tert-butoxycarbonylamino-propyl)-6-chloro-2-fluoro-phenoxy]-2-fluoro-benzoic acid methyl ester (2.9 g, 1H NMR >95%, 6.36 mmol, 19.3% yield). 1H NMR (270 MHz, CDCl₃): 7.41-7.38 (1H, m), 7.21 (1H, m), 7.12-7.00 (3H, m), 4.93 (1H, br s), 4.70 (1H, m), 3.89 (3H, s), 1.80-1.70 (2H, m), 1.40 (9H, br s), 0.90 (3H, t).

### Step 2

To a solution of 5-[3-((R)-1-tert-butoxycarbonylamino-propyl)-6-chloro-2-fluoro-phenoxy]-2-fluoro-benzoic acid methyl ester (2.90 g, 6.36 mmol, 1.0 eq) in THF (70 ml) was added LiOH.H₂O (2.67 g, 63.6 mmol, 10 eq) in water (53 ml) and the mixture stirred vigorously at room temperature overnight after which time analysis (HPLC) indicated complete hydrolysis. The THF was removed in vacuo, the remaining aqueous acidified to pH 45 with saturated aqueous citric acid solution (50 ml) and extracted with EtOAc (3×50 ml). The combined organics were dried (MgSO₄), filtered and concentrated in vacuo to give 5-[3-((R)-1-tert-butoxycarbonylamino-propyl)-6-chloro-2-fluoro-phenoxy]-2-fluoro-benzoic acid as a white solid (2.56 g, 1H NMR >95%, 5.79 mmol, 91% yield). 1H NMR (270 MHz, CDCl₃): 7.41-7.38 (1H, m), 7.23 (1H, m), 7.14-7.09 (3H, m), 4.93 (1H, br s), 4.72 (1H, m), 1.81-1.71 (2H, m), 1.40 (9H, br s), 0.90 (3H, t).

### Step 3 General Procedure

To a solution of 5-[3-((R)-1-tert-butoxycarbonylamino-propyl)-6-chloro-2-fluoro-phenoxy]-2-fluoro-benzoic acid (640 mg, 1.45 mmol, 1.0 eq) in THF (13 ml) was added ammonia (16.7 mmol, 11.5 eq), ⁱPrNEt₂ (1.9 ml, 11.0 mmol, 7.5 eq) and HATU (826 mg, 2.17 mmol, 1.5 eq) and the mixture stirred overnight, after which time analysis (HPLC) showed complete conversion to product. The mixture was diluted EtOAc (30 ml) and washed with water (2×10 ml). The aqueous was extracted with EtOAc (20 ml) and the combined organics dried (MgSO₄), filtered and concentrated in vacuo. The residue was redissolved in EtOAc (20 ml) and washed with water (2×10 ml), 10% aq K₂CO₃ (10 ml) and brine (10 ml) then dried (MgSO₄), filtered and concentrated in vacuo to give {(R)-1-[3-(3-carbamoyl-4-fluoro-phenoxy)-4-chloro-2-fluoro-phenyl]-propyl}-carbamic acid tert-butyl ester

1H NMR (270 MHz, CDCl₃): 7.46 (1H, dd), 7.18 (1H, dd), 7.09-6.92 (3H, m), 5.23 (1H, d), 4.66 (1H, br s), 1.75-1.64 (2H, m), 1.35 (9H, br s), 0.85 (3H, t).

### Step 4 General Procedure

The amide obtained from Step 3 was dissolved in EtOAc (10 ml), 4 M HCl in EtOAc (15 ml) added and the mixture stirred for 1 hour, after which time further 4 M HCl in EtOAc (5 ml) was added. The mixture was stirred for an additional 1 hour after which time analysis (HPLC) indicated complete deprotection. The mixture was concentrated in vacuo, then the residue azeotroped with Et₂O followed by 11 heptane.Et₂O to give (R)-1-[3-(3-carbamoyl-4-fluoro-phenoxy)-4-chloro-2-fluoro-phenyl]-propylamine hydrochloride as a solid, 585 mg, 1H NMR 93% (7% solvents), 1.44 mmol, 99% yield.

### Example 374

### 5-[3-((R)-1-Amino-propyl)-6-chloro-2-fluoro-phenoxyl-2-fluoro-N-methyl-benzamide hydrochloride

### Step 1

Following the method described in Example 373 Step 3, but substituting methylamine for ammonia, gave {(R)-1-[4-Chloro-2-fluoro-3-(4-fluoro-3-methylcarbamoyl-phenoxy)-phenyl]propyl}-carbamic acid tert-butyl ester

1H NMR (270 MHz, CDCl₃): 7.45 (1H, dd), 7.18 (1H, dd), 7.09-7.02 (3H, m), 5.24 (1H, d), 4.66 (1H, br s), 2.93 (3H, s), 1.75-1.65 (2H, m), 1.36 (9H, br s), 0.86 (3H, t).

### Step 2

Deprotection of the product of Step 1, following the procedure of Example 373 Step 4, gave (R)-1-[4-chloro-2-fluoro-3-(4-fluoro-3-methylcarbamoyl-phenoxy)-phenyl]-propylamine hydrochloride, 610 mg, ¹H NMR 92% (8% solvents), 1.43 mmol, 98% yield.

### Example 375

### {5-[3-((R)-1-Amino-propyl)-6-chloro-2-fluoro-phenoxyl-2-fluoro-phenyl}-morpholin-4-yl-methanone hydrochloride

### Step 1

Following the method described in Example 373 Step 3, but substituting morpholine for ammonia, gave ((R)-1-{4-chloro-2-fluoro-3-[4-fluoro-3-(morpholine-4-carbonyl)-phenoxy]-phenyl}-propyl)-carbamic acid tert-butyl ester.

1H NMR (270 MHz, CDCl₃): 7.18 (1H, dd), 7.09-7.02 (2H, m), 6.87-6.82 (2H, m), 5.25 (1H, d), 4.65 (1H, br s), 3.71 (4H, br s), 3.60 (2H, dd), 3.34-3.30 (2H, m), 1.74-1.64 (2H, m), 1.36 (9H, br s), 0.85 (3H, t).

### Step 2

Deprotection of the product of Step 1, following the procedure of Example 373 Step 4, gave (R)-1-{4-chloro-2-fluoro-3-[4-fluoro-3-(morpholine-4-carbonyl)-phenoxy]-phenyl}-propylamine hydrochloride, 500 mg, 1H NMR 95% (5% solvents), 1.06 mmol, 73% yield.

### Example 376

### 5-[3-((R)-1-Amino-propyl)-6-chloro-2-fluoro-phenoxy]-2-fluoro-N,N-dimethyl-benzamide hydrochloride

### Step 1

Following the method described in Example 373 Step 3, but substituting dimethylamine for ammonia, gave {(R)-1-[4-chloro-3-(3-dimethylcarbamoyl-4-fluoro-phenoxy)-2-fluoro-phenyl]-propyl}-carbamic acid tert-butyl ester.

1H NMR (270 MHz, CDCl₃): 7.19 (1H, dd), 7.09-6.96 (2H, m), 6.89-6.81 (2H, m), 5.13 (1H, d), 4.67 (1H, br s), 3.06 (3H, s), 2.91 (3H, s), 1.77-1.66 (2H, m), 1.37 (9H, br s), 0.87 (3H, t).

### Step 2

Deprotection of the product of Step 1, following the procedure of Example 373 Step 4, gave (R)-1-[4-chloro-3-(3-dimethylcarbamoyl-4-fluoro-phenoxy)-2-fluoro-phenyl]-propylamine hydrochloride, 615 mg ¹H NMR 90% (10% solvents), 1.37 mmol, 94% yield.

### Example 377

### (R)-1-[4-Chloro-2-fluoro-3-(pyrimidin-4-yloxy)-phenyl]-propylamine hydrochloride

### Step 1

To a mixture of Key Intermediate KI-3a, (R)-2-methyl-propane-2-sulfinic acid

[(R)-1-(4-chloro-2-fluoro-3-hydroxy-phenyl)-propyl]-amide, (500 mg, 1.62 mmol, 1.0 eq) and 1,4-dioxane (30 ml) was added potassium tert-butoxide (220 mg, 1.96 mol, 1.2 eq). After 30 min, 4,6-dichloropyrimidine (300 mg, 2.01 mmol, 1.24 eq) was added and the reaction heated at 100° C. for 20 hours. The dioxaneproduct was decanted off and concentrated in vacuo. The residue was partitioned between 10% citric acid solution (30 ml) and DCM (60 ml). The organic layer was washed with 10% K₂CO₃ solution (30 ml), dried, filtered and concentrated onto silica (2 g). The material was purified by column chromatography on silica (30 g), eluting with 1:1 heptanesEtOAc. The product fractions were combined and concentrated to give (R)-2-methyl-propane-2-sulfinic acid {(R)-1-[4-chloro-3-(6-chloro-pyrimidin-4-yloxy)-2-fluoro-phenyl]-propyl}-amide (560 mg, ¹H NMR >95%, 1.33 mmol, 82% yield). 1H NMR (270 MHz, CDCl₃): 8.26 (1H, s), 7.25 (2H, dd), 7.13 (1H, s), 4.52 (1H, q), 3.50 (1H, d), 2.05-1.70 (2H, m), 1.20 (9H, s), 0.87 (3H, t).

### Step 2

To a solution of (R)-2-methyl-propane-2-sulfinic acid {(R)-1-[4-chloro-3-(6-chloro-pyrimidin-4-yloxy)-2-fluoro-phenyl]-propyl}-amide (490 mg, 1.17 mmol) in MeOH (15 ml) was added N,N-diisopropylethylamine (0.25 ml, 1.43 mmol, 1.23 eq) and 10% PdC (50% wet, 0.1 g). The reaction was stirred vigorously under a hydrogen atmosphere for 2 hours. The catalyst was removed by filtration and the filtrate concentrated in vacuo. The crude material was adsorbed onto silica (1 g) and purified by column chromatography on silica (20 g), eluting with 1:1 heptanesEtOAc. The product fractions were combined and concentrated to give (R)-2-methyl-propane-2-sulfinic acid {(R)-1-[4-chloro-2-fluoro-3-(pyrimidin-4-yloxy)-phenyl]-propyl}-amide (290 mg, 1H NMR >80%, 0.60 mmol, 51% yield).

1H NMR (270 MHz, CDCl₃): 8.72 (1H, s), 8.64 (1H, d), 7.30-7.15 (2H, m), 7.09 (1H, dd), 4.52 (1H, q), 3.55 (1H, d), 2.10-1.40 (2H, m), 1.21 (9H, s), 0.88 (3H, t).

### Step 3

To a solution of (R)-2-methyl-propane-2-sulfinic acid {(R)-1-[4-chloro-2-fluoro-3-(pyrimidin-4-yloxy)-phenyl]-propyl}-amide (290 mg, 0.75 mmol) in EtOAc (5 ml) was added 2 M HCl in EtOAc (2 ml, 4.2 mmol). After stirring for 1 hour, the solid was filtered off and washed with EtOAc (5 ml) and Et₂O (5 ml). The material was dried to give (R)-1-[4-chloro-2-fluoro-3-(pyrimidin-4-yloxy)-phenyl]-propylamine hydrochloride (182 mg, 0.57 mmol, 77% yield).

### Example 378

### 6-[3-((R)-1-Amino-propyl)-6-chloro-2-fluoro-phenoxy]-pyrimidin-4-ylamine hydrochloride

### Step 1

A solution of the compound of Example 377 Step 1, (R)-2-methyl-propane-2-sulfinic acid {(R)-1-[4-chloro-3-(6-chloro-pyrimidin-4-yloxy)-2-fluoro-phenyl]-propyl}-amide, (800 mg, 1.90 mmol, 1.0 eq) in 7N NH₃MeOH (15 ml) was heated in a sealed tube at 110° C. for 2 days. The solvent was removed in vacuo and the crude material purified by column chromatography on silica (6 g), eluting with 1:1 heptanesEtOAc. The product fractions were combined and concentrated to give (R)-2-methyl-propane-2-sulfinic acid {(R)-1-[3-(6-amino-pyrimidin-4-yloxy)-4-chloro-2-fluoro-phenyl]-propyl}-amide (136 mg, ¹H NMR >95% excluding solvent, 63% active, 0.21 mmol, 11% yield). 1H NMR (270 MHz, CDCl₃): 8.18 (1H, s), 7.35-7.15 (2H, m), 6.02 (1H, s), 4.45 (1H, q), 3.70 (1H, d), 2.10-170 (2H, m), 1.21 (9H, s), 0.85 (3H, t).

### Step 2

To a solution of (R)-2-methyl-propane-2-sulfinic acid {(R)-1-[3-(6-amino-pyrimidin-4-yloxy)-4-chloro-2-fluoro-phenyl]-propyl}-amide (136 mg, 0.339 mmol) in EtOAc (10 ml) was added 2.1M HCl in EtOAc (2 ml, 4.2 mmol). After stirring for 1 hour, the solid was filtered off and washed with EtOAc (2 ml) and Et₂O (2 ml). The material was dried to give 6-[3-((R)-1-amino-propyl)-6-chloro-2-fluoro-phenoxy]-pyrimidin-4-ylamine hydrochloride (79 mg, 0.24 mmol, 70% yield).

### Example 379

### (R)-1-[4-Chloro-2-fluoro-3-(pyridazin-3-yloxy)-phenyl]-propylamine hydrochloride

### Step 1

To a mixture of Key Intermediate KI-3a, (R)-2-methyl-propane-2-sulfinic acid [(R)-1-(4-chloro-2-fluoro-3-hydroxy-phenyl)-propyl]-amide, (500 mg, 1.62 mmol, 1.0 eq) and 1,4-dioxane (30 ml) was added potassium tert-butoxide (220 mg, 1.96 mol, 1.2 eq). After 30 min, 3,6-dichloropyridazine (730 mg, 4.90 mmol, 3.02 eq) was added and the reaction heated at 100° C. for 72 hours. The reaction was concentrated in vacuo and the residue partitioned between water (20 ml) and DCM (40 ml). The organic layer was dried, filtered and concentrated onto silica (2 g). The material was purified by column chromatography on silica (20 g), eluting with 1:1 heptanesEtOAc. The product fractions were combined and concentrated to give (R)-2-methyl-propane-2-sulfinic acid {(R)-1-[4-chloro-3-(6-chloro-pyridazin-3-yloxy)-2-fluoro-phenyl]-propyl}-amide (503 mg, ¹H NMR >95%, 1.20 mmol, 74% yield). 1H NMR (270 MHz, CDCl₃): 7.55 (1H, d), 7.33 (1H, d), 7.30-7.15 (2H, m), 4.50 (1H, q), 3.50 (1H, d), 2.05-1.40 (2H, m), 1.20 (9H, s), 0.84 (3H, t).

### Step 2

To (R)-2-methyl-propane-2-sulfinic acid {(R)-1-[4-chloro-3-(6-chloro-pyridazin-3-yloxy)-2-fluoro-phenyl]-propyl}-amide (500 mg, 1.19 mmol, 1.0 eq) in MeOH (10 ml) was added N,N-diisopropylethylamine (0.1 ml, 0.57 mmol, 0.48 eq) and 10% PdC (50% wet, 0.1 g). The reaction was stirred vigorously under a hydrogen atmosphere for 2 hours. Additional 10% PdC (50% wet, 0.1 g) was added and the reaction stirred for a further 16 hours. The catalyst was removed by filtration and the filtrate concentrated in vacuo. The crude material was adsorbed onto silica (1 g) and purified by column chromatography on silica (15 g), eluting with 1:3 heptanesEtOAc. The product fractions were combined and concentrated to give (R)-2-methyl-propane-2-sulfinic acid {(R)-1-[4-chloro-2-fluoro-3-(pyridazin-3-yloxy)-phenyl]-propyl}-amide (403 mg, 1H NMR >90%, 0.94 mmol, 79% yield). 1H NMR (270 MHz, CDCl₃): 8.93 (1H, dd), 7.28 (1H, dd), 7.31 (1H, dd), 7.27-7.15 (2H, m), 4.51 (1H, q), 3.55 (1H, d), 2.05-1.70 (2H, m), 1.20 (9H, s), 0.85 (3H, t).

### Step 3

To a solution of (R)-2-methyl-propane-2-sulfinic acid {(R)-1-[4-chloro-2-fluoro-3-(pyridazin-3-yloxy)-phenyl]-propyl}-amide (310 mg, 0.80 mmol) in EtOAc (5 ml) was added 2.1 M HCl in EtOAc (2 ml, 4.2 mmol). After stirring for 1 hour, the solid was filtered off and washed with EtOAc (5 ml) and Et₂O (5 ml). The material was dried to give (R)-1-[4-chloro-2-fluoro-3-(pyridazin-3-yloxy)-phenyl]-propylamine hydrochloride (186 mg, 0.58 mmol, 73% yield).

### Example 380

### (R)-1-[4-Chloro-2-fluoro-3-(pyrazin-2-yloxy)-phenyl]-propylamine hydrochloride

### Step 1

To a flask was charged Key Intermediate KI-3a, (R)-2-methyl-propane-2-sulfinic acid [(R)-1-(4-chloro-2-fluoro-3-hydroxy-phenyl)-propyl]-amide, (1.00 g, 3.25 mmol, 1.0 eq), chloropyrazine (0.744 g, 6.50 mmol, 2.0 eq), Cs₂CO₃ (2.22 g, 6.81 mmol, 2.1 eq) and DMSO (40 ml) and the stirred reaction was heated to 110° C. overnight. To this, more chloropyrazine (0.372 g, 3.25 mmol, 1.0 eq) was added stirred at 110° C. for a further 7 hours. To this, chloropyrazine (0.653 g, 4.51 mmol, 1.4 eq) and Cs₂CO₃ (1.70 g, 5.22 mmol, 1.6 eq) were added stirred at 110° C. overnight. The reaction was cooled to RT, poured into water (400 ml), extracted with 15% heptaneEtOAc (2×200 ml). The organics were washed with water (3×200 ml) and brine (200 ml), dried over MgSO₄, filtered and concentrated in vacuo. The crude material was purified via column chromatography (silica, 50 g) eluting with 1:1 EtOAc:heptanes to give (R)-2-methyl-propane-2-sulfinic acid {(R)-1-[4-chloro-2-fluoro-3-(pyrazin-2-yloxy)-phenyl]-propyl}-amide (580 mg, ¹H NMR >90%, 1.35 mmol, 42% yield). 1H NMR (270 MHz, CDCl₃): 8.57 (1H, d), 8.30 (1H, d), 8.03 (1H, dd), 7.29-7.18 (2H, m), 4.53 (1H, q), 3.50 (1H, d), 2.06-1.74 (2H, m), 1.21 (9H, s), 0.91 (3H, t).

### Step 2

To a solution of (R)-2-methyl-propane-2-sulfinic acid {(R)-1-[4-chloro-2-fluoro-3-(pyrazin-2-yloxy)-phenyl]-propyl}-amide (600 mg, 1.55 mmol, 1.0 eq) in EtOAc (45 ml) was added 2.1 M HCl in EtOAc (4.39 ml, 9.22 mmol, 5.9 eq) slowly and the mixture was stirred at RT for 2 hours. The reaction was concentrated in vacuo and the residue was slurried in 3:1 heptane:Et₂O (45 ml) overnight. The solids were filtered, washed with 3:1 heptane:Et₂O (2×25 ml) and dried in vacuo at 35° C. overnight to give (R)-1-[4-chloro-2-fluoro-3-(pyrazin-2-yloxy)-phenyl]-propylamine hydrochloride (409 mg, ¹H NMR >95%, 1.29 mmol, 83% yield).

### Example 381

### 5-[3-((R)-1-Amino-propyl)-6-chloro-2-fluoro-phenoxy]-pyrazin-2-ylamine hydrochloride

### Step 1

A mixture of Key Intermediate KI-3a, (R)-2-methyl-propane-2-sulfinic acid [(R)-1-(4-chloro-2-fluoro-3-hydroxy-phenyl)-propyl]-amide, (1.25 g, 4.06 mmol, 1 eq), MeCN (75 ml), K₂CO₃ (1.7 g, 12.3 mmol, 3 eq), sodium iodide (75 mg, 0.50 mmol, 0.12 eq) and methyl 5-chloropyrazinecarboxylate (1440 g, 8.34 mmol, 2.1 eq) was stirred at 40° C. for 24 hours. The solvent was removed in vacuo and the crude material partitioned between DCM (50 ml) and water (30 ml). The organic layer was dried, filtered and adsorbed onto silica (3 g). Purification by column chromatography on silica (50 g), eluting with 1:2 up to 1:1 heptanesEtOAc afforded 1420 mg crude (R)-5-{6-Chloro-2-fluoro-3-[(R)-1-((R)-2-methyl-propane-2-sulfinylamino)-propyl]-phenoxy}-pyrazine-2-carboxylic acid methyl ester (contained 5% intermediate 3 by NMR). The material was dissolved in DCM (30 ml) and washed with 10% K₂CO₃ (2×20 ml) before being dried, filtered and concentrated to give (R)-5-{6-chloro-2-fluoro-3-[(R)-1-(2-methyl-propane-2-sulfinylamino)-propyl]-phenoxy}-pyrazine-2-carboxylic acid methyl ester (1190 mg, ¹H NMR >95%, 2.67 mmol, 66% yield). 1H NMR (270 MHz, CDCl₃): 8.78 (1H, s), 8.64 (1H, s), 7.25 (2H, dd), 4.52 (1H, q), 4.01 (3H, s), 3.50 (1H, d), 2.10-1.70 (2H, m), 1.22 (9H, s), 0.89 (3H, t).

### Step 2

(R)-5-{6-Chloro-2-fluoro-3-[(R)-1-(2-methyl-propane-2-sulfinylamino)-propyl]-phenoxy}-pyrazine-2-carboxylic acid methyl ester (1190 mg, 2.68 mmol, 1 eq) was dissolved in THF (10 ml) before water (10 ml) and LiOH.H₂O (500 mg, 11.96 mmol, 4.5 eq) were added. After 1 hour at room temperature, the THF was removed in vacuo and the aqueous washed with Et₂O (10 ml). The aqueous layer was acidified to pH 4 with 10% citric acid solution (20 ml) and extracted with EtOAc (30 ml). The combined organic layers were washed with sat. brine (30 ml) before being dried, filtered and concentrated to give (R)-5-{6-chloro-2-fluoro-3-[(R)-1-(2-methyl-propane-2-sulfinylamino)-propyl]-phenoxy}-pyrazine-2-carboxylic acid (1.9 g, ¹H NMR >95% excluding solvent, 53% active, 2.33 mmol, 87% yield). 1H NMR (270 MHz, MeOD): 8.77 (1H, s), 8.68 (1H, s), 7.50-7.30 (2H, m), 4.48 (1H, t), 2.00-1.40 (2H, m), 1.20 (9H, s), 0.94 (3H, t).

### Step 3

To (R)-5-{6-chloro-2-fluoro-3-[(R)-1-(2-methyl-propane-2-sulfinylamino)-propyl]-phenoxy}-pyrazine-2-carboxylic acid (1.2 g, 2.79 mmol, 1.0 eq) was added tert-butanol (10 ml) and triethylamine (320 mg, 3.16 mmol, 1.13 eq) and the mixture heated to 80° C. Diphenylphosphoryl azide (800 mg, 2.91 mmol, 1.04 eq) was added and the reaction heated for 16 hours. Additional diphenylphosphoryl azide (300 mg, 1.09 mmol, 0.39 eq) was charged and after an additional 5 hours the reaction was cooled and tert-butanol removed in vacuo. The crude material was partitioned between DCM (20 ml) and water (20 ml). The organic layer was washed with sat. brine (10 ml), dried, filtered and concentrated. Purification by column chromatography on silica (60 g), eluting with 1:2 heptanesEtOAc gave (R)-(5-{6-chloro-2-fluoro-3-[(R)-1-(2-methyl-propane-2-sulfinylamino)-propyl]-phenoxy}-pyrazin-2-yl)-carbamic acid tert-butyl ester (415 mg, ¹H NMR >95%, 0.83 mmol, 30% yield). 1H NMR (270 MHz, CDCl₃): 8.66 (1H, s), 8.18 (1H, s), 7.30-7.10 (2H, m), 4.51 (1H, q), 3.50 (1H, d), 2.05-1.40 (2H, m), 1.51 (9H, s), 1.21 (9H, s), 0.86 (3H, t).

### Step 4

(R)-(5-{6-Chloro-2-fluoro-3-[(R)-1-(2-methyl-propane-2-sulfinylamino)-propyl]-phenoxy}-pyrazin-2-yl)-carbamic acid tert-butyl ester (415 mg, 0.83 mmol, 1.0 eq) was dissolved in EtOAc (5 ml) and 4M HCl in EtOAc (10 ml, 40 mmol, 48.2 eq) was added. HPLC analysis showed the deprotection was not complete, therefore 4 M HCl in EtOAc (3 ml, 12 mmol, 14.5 eq) was added and the mixture stirred for 1 hour. After this time the solids were filtered off and washed with Et₂O (5 ml). The material was dried at 40° C. to give 5-[3-((R)-1-amino-propyl)-6-chloro-2-fluoro-phenoxy]-pyrazin-2-ylamine hydrochloride (201 mg, 0.68 mmol, 73% yield).

### Example 382

### (R)-1-[4-Chloro-2-fluoro-3-(pyrimidin-2-yloxy)-phenyl]-propylamine

### Step 1

A mixture of Key Intermediate KI-3a, (R)-2-methyl-propane-2-sulfinic acid [(R)-1-(4-chloro-2-fluoro-3-hydroxy-phenyl)-propyl]-amide, (0.700 g, 2.27 mmol, 1.0 eq), 2-chloropyrimidine (0.313 g, 2.73 mmol, 1.2 eq) and K₂CO₃ (1.57 g, 11.4 mol, 5.0 eq) in DMF (28 ml) was stirred at 110° C. for 5 hour. The reaction was cooled to RT, poured into water (100 ml), extracted with 15% heptaneEtOAc (2×100 ml), washed with water (2×100 ml) then brine (100 ml), dried over MgSO₄, filtered and concentrated in vacuo. The crude residue was purified via column chromatography (silica, 40 g) eluting with 1:1 heptane:EtOAc to give (R)-2-methyl-propane-2-sulfinic acid {(R)-1-[4-chloro-2-fluoro-3-(pyrimidin-2-yloxy)-phenyl]-propyl}-amide (470 mg, ¹H NMR >95%, 1.22 mmol, 45% yield). 1H NMR (270 MHz, CDCl₃): 8.55 (2H, d), 7.28-7.18 (2H, m), 7.08 (1H, t), 4.56 (1H, q), 3.51 (1H, d), 2.06-1.74 (2H, m), 1.21 (9H, s), 0.88 (3H, t).

### Step 2

To a solution of (R)-2-methyl-propane-2-sulfinic acid {(R)-1-[4-chloro-2-fluoro-3-(pyrimidin-2-yloxy)-phenyl]-propyl}-amide (415 mg, 1.17 mmol, 1.0 eq) in EtOAc (50 ml) was added 2.1M HCl in EtOAc (1.66 ml, 3.49 mmol, 3.0 eq) and stirred at RT for 1.5 hours. The reaction was concentrated in vacuo and azeotroped with toluene (20 ml). The residue was slurried in 3:1 heptane:Et₂O (20 ml) for 2 hours, the solids were filtered and washed with 3:1 heptane:Et₂O (10 ml). The solids were dried in an oven under vacuum at 30° C. for ca. 60 hours under vacuum to give (R)-1-[4-chloro-2-fluoro-3-(pyrimidin-2-yloxy)-phenyl]-propylamine hydrochloride (358 mg, ¹H NMR >95%, 1.13 mmol, 96% yield).

### Example 383

### 2-[3-((R)-1-Amino-propyl)-6-chloro-2-fluoro-phenoxy]-pyrimidin-5-ylamine hydrochloride

### Step 1

To a solution of Key Intermediate KI-3a, (R)-2-methyl-propane-2-sulfinic acid

[(R)-1-(4-chloro-2-fluoro-3-hydroxy-phenyl)-propyl]-amide (700 mg, 2.27 mmol, 1 eq) in MeCN (42 ml) was charged potassium carbonate (952 mg, 6.88 mmol, 3.0 eq), sodium iodide (42 mg, 0.280 mmol, 0.12 eq) and 2-chloro-5-nitropyridimidine (728 mg, 4.56 mmol, 2.0 eq). After 16 hours at room temperature; the solids were filtered off and washed with MeCN (10 ml). The liquors were concentrated in vacuo and the crude material purified by column chromatography on silica (50 g), eluting with 1:1 heptanesEtOAc. The product fractions were combined and concentrated followed by a Et₂O (10 ml) strip to give (R)-2-methyl-propane-2-sulfinic acid {(R)-1-[4-chloro-2-fluoro-3-(5-nitro-pyrimidin-2-yloxy)-phenyl]-propyl}-amide (901 mg, ¹H NMR >95%, 2.09 mmol, 92% yield). 1H NMR (270 MHz, CDCl₃): 8.77 (1H, s), 8.63 (1H, s), 7.26 (2H, dd), 4.51 (1H, q), 3.52 (1H, d), 2.05-1.65 (2H, m), 1.21 (9H, s), 0.89 (3H, t).

### Step 2

To a solution of (R)-2-methyl-propane-2-sulfinic acid {(R)-1-[4-chloro-2-fluoro-3-(5-nitro-pyrimidin-2-yloxy)-phenyl]-propyl}-amide (800 mg, 1.86 mmol, 1.0 eq) in MeOH (8 ml) was charged water (8 ml), ammonium chloride (500 mg, 9.35 mmol, 5.0 eq) and powdered iron (520 mg, 9.35 mmol, 5.0 eq). The reaction was heated at 60° C. for 1 hour, the solids filtered off and washed with MeOH (20 ml). The solvent was removed in vacuo and the solids filtered off and washed with water (5 ml). The crude solid was partitioned between EtOAc (100 ml) and water (20 ml), the organic layer dried, filtered and concentrated to give 800 mg crude solid. The material was purified by column chromatography on silica (20 g), eluting with 100% EtOAc. The product fractions were combined to give (R)-2-methyl-propane-2-sulfinic acid {(R)-1-[3-(5-amino-pyrimidin-2-yloxy)-4-chloro-2-fluoro-phenyl]-propyl}-amide (496 mg, ¹H NMR >95% excluding solvents, 76% active, 0.94 mmol, 51% yield). ¹H NMR (270 MHz, CDCl₃): 8.00 (2H, s), 7.23-7.10 (2H, m), 4.56 (1H, q), 3.61 (2H, bs), 3.54 (1H, d), 2.05-1.70 (2H, m), 1.20 (9H, s), 0.86 (3H, t).

### Step 3

To (R)-2-methyl-propane-2-sulfinic acid {(R)-1-[3-(5-amino-pyrimidin-2-yloxy)-4-chloro-2-fluoro-phenyl]-propyl}-amide (490 mg, 1.22 mmol, 1.0 eq) in EtOAc (10 ml) was added 2.1M HCl in EtOAc (3 ml, 6.3 mmol, 5.16 mmol). After 1 hour, the solids were filtered off and washed with Et₂O (5 ml) and heptanes (5 ml). The solid was dried at 30° C. in a vacuum oven to give 2-[3-((R)-1-amino-propyl)-6-chloro-2-fluoro-phenoxy]-pyrimidin-5-ylamine hydrochloride (320 mg, 0.96 mmol, 79% yield)—see table 2.

### Example 384

### (R)-1-[3-(Benzothiazol-2-yloxy)-4-chloro-2-fluoro-phenyl]-propylamine hydrochloride

### Step 1

To a reaction tube was charged Key Intermediate KI-3a, (R)-2-methyl-propane-2-sulfinic acid [(R)-1-(4-chloro-2-fluoro-3-hydroxy-phenyl)-propyl]-amide, (700 mg, 2.27 mmol, 1.0 eq), 2-chlorobenzo[d]thiazole (463 mg, 2.73 mmol, 1.2 eq), K₂CO₃ (1.57 g, 11.4 mmol, 5.0 eq) and DMF (12 ml) and the reaction was stirred under N₂ at 100° C. overnight. The reaction was cooled to RT, poured into H₂O (25 ml) and extracted with DCM (2×25 ml). The organics were concentrated in vacuo, taken up in 10% heptaneEtOAc (20 ml) and washed with H₂O (20 ml). The organics were dried over MgSO₄, filtered and concentrated in vacuo. The residue was purified by column chromatography (silica, 40 g) packed in DCM and eluted with DCM followed by 5% MeOHDCM. Product containing fractions were combined and concentrated in vacuo to give (R)-2-methyl-propane-2-sulfinic acid {(R)-1-[3-(benzothiazol-2-yloxy)-4-chloro-2-fluoro-phenyl]-propyl}-amide (600 mg, ¹H NMR >90%, 1.22 mmol, 54% yield). 1H NMR (270 MHz, CDCl₃): 7.71-7.65 (2H, m), 7.40-7.21 (4H, m), 4.48 (1H, q), 3.56 (1H, d), 2.27-1.74 (2H, m), 1.22 (9H, s), 0.91 (3H, t).

### Step 2

To a solution of (R)-2-methyl-propane-2-sulfinic acid {(R)-1-[3-(benzothiazol-2-yloxy)-4-chloro-2-fluoro-phenyl]-propyl}-amide (550 mg, 1.30 mmol, 1.0 eq) in MeOH (12 ml) was added 2.1M HCl in EtOAc (1.2 ml, 2.52 mmol, 1.9 eq). The mixture was stirred at RT for 1 hour and then concentrated in vacuo. To the residue was added 3:1 heptane:Et₂O (15 ml) and the mixture was stirred overnight at RT. The solid was filtered, washed with 3:1 heptane:Et₂O (15 ml) and dried in a vacuum oven for 6 hours at 35° C. to give (R)-1-[3-(benzothiazol-2-yloxy)-4-chloro-2-fluoro-phenyl]-propylamine hydrochloride as an off white solid (338 mg, ¹H NMR >95%, 0.906 mmol, 70% yield).

### Example 385

### 2-[3-((R)-1-Amino-propyl)-6-chloro-2-fluoro-phenoxy]-benzothiazol-5-yl amine hydrochloride

### Step 1

To a N₂ purged flask was added Key Intermediate KI-3a, (R)-2-methyl-propane-2-sulfinic acid [(R)-1-(4-chloro-2-fluoro-3-hydroxy-phenyl)-propyl]-amide, (700 mg, 2.27 mmol, 1.0 eq), potassium tert-butoxide (385 mg, 3.43 mmol, 1.5 eq), 2-chloro-5-nitrobenzo[d]thiazole (738 mg, 3.43 mmol, 1.5 eq) and 1,4-dioxane (42 ml). The stirred mixture was heated quickly to 100° C. and stirred for 48 hours. The mixture was cooled to RT and concentrated in vacuo. The organics were extracted into DCM (3×200 ml) and concentrated in vacuo. The crude material was purified via column chromatography (silica, 55 g) eluting with 2% MeOHDCM. The product containing fractions were combined and concentrated giving (R)-2-methyl-propane-2-sulfinic acid {(R)-1-[4-chloro-2-fluoro-3-(5-nitro-benzothiazol-2-yloxy)-phenyl]propyl}-amide as a yellow oil (970 mg, 1H NMR >95% excluding solvent, 90% active, 1.80 mmol, 79% yield). 1H NMR (270 MHz, CDCl₃): 8.52 (1H, d), 8.19 (1H, dd), 7.85 (1H, d), 7.35-7.27 (2H, m), 4.55 (1H, q), 2.08-1.75 (2H, m), 1.23 (9H, s), 0.92 (3H, t).

### Step 2

To a solution of (R)-2-methyl-propane-2-sulfinic acid {(R)-1-[4-chloro-2-fluoro-3-(5-nitro-benzothiazol-2-yloxy)-phenyl]propyl}-amide (800 mg, 1.65 mmol, 1.0 eq) in MeOH (20 ml) was added NH₄Cl (440 mg, 8.23 mmol, 5.0 eq) dissolved in H₂O (20 ml) and the mixture was stirred under N₂ at 40° C. To this was added iron powder (460 mg, 8.23 mmol, 5.0 eq) and the reaction heated to 76° C. for 1 hour. The reaction was cooled to RT and stirred overnight. The mixture was filtered, washed with MeOH (200 ml) and concentrated in vacuo. The residue was dissolved in H₂O (100 ml) and extracted with EtOAc (2×150 ml). The combined organics were washed with H₂O (100 ml) and brine (100 ml), dried over MgSO₄, filtered and concentrated in vacuo. The crude residue was purified by column chromatography (silica, 26 g), packed in DCM and eluted with 50% DCMEtOAc. The product fraction were combined and concentrated to give (R)-2-methyl-propane-2-sulfinic acid {(R)-1-[3-(5-amino-benzothiazol-2-yloxy)-4-chloro-2-fluoro-phenyl]-propyl}-amide as a yellow solid (511 mg, ¹H NMR >95% excluding solvent, 90% active, 1.01 mmol, 61% yield). 1H NMR (270 MHz, CDCl₃): 7.41 (1H, d), 7.30-7.21 (2H, m), 6.98 (1H, d), 6.66 (1H, dd), 4.11 (1H, q), 3.74 (2H, bs), 3.56 (1H, d), 2.09-1.72 (2H, m), 1.21 (9H, s), 0.89 (3H, t).

### Step 3

To a solution of (R)-2-methyl-propane-2-sulfinic acid {(R)-1-[3-(5-amino-benzothiazol-2-yloxy)-4-chloro-2-fluoro-phenyl]-propyl}-amide (450 mg, 0.995 mmol, 1.0 eq) in EtOAc (40 ml) was added 2.1M HCl in EtOAc (2.9 ml, 6.09 mmol, 6.1 eq). The reaction was stirred at room temp for 1.5 hours. The reaction was concentrated in vacuo and redissolved in EtOAc (40 ml) and 2.1M HCl in EtOAc (2 ml, 4.20 mml, 4.2 eq) was added. The mixture was stirred for 2 hours at RT and the white precipitate was filtered and washed with 4:1 EtOAc:Et₂O (3 ml). The solid was dried in a vacuum oven at 35° C. overnight to provide 2-[3-((R)-1-amino-propyl)-6-chloro-2-fluoro-phenoxy]-benzothiazol-5-yl amine hydrochloride as an off white solid (340 mg, ¹H NMR >95%, 0.876 mmol, 88% yield).

### Example 386

### (R)-1-[4-Chloro-2-fluoro-3-(thiazolo[4,5-d]pyridin-2-yloxy)-phenyl]-propylamine hydrochloride

### Step 1

To a flask was charged Key Intermediate KI-3a, (R)-2-methyl-propane-2-sulfinic acid [(R)-1-(4-chloro-2-fluoro-3-hydroxy-phenyl)-propyl]-amide, (0.700 g, 2.27 mmol, 1.0 eq), Cs₂CO₃ (1.48 g, 4.55 mmol, 2.2 eq), 2-chlorothiazolo[4,5-c]pyridine (0.466 g, 2.73 mmol, 1.2 eq) and DMSO (28 ml). The mixture was stirred at 110° C. for 1.5 hours then allowed to cool to RT. The reaction was diluted with 15% heptaneEtOAc (200 ml), washed with water (3×200 ml) then brine (200 ml), dried over MgSO₄, filtered and concentrated in vacuo. The crude material was purified via column chromatography (silica, 45 g) eluting with 2:1 heptane:EtOAc to give (R)-2-methyl-propane-2-sulfinic acid {(R)-1-[4-chloro-2-fluoro-3-(thiazolo[4,5-c]pyridin-2-yloxy)-phenyl]-propyl}-amide (500 mg, ¹H NMR >80%, 0.905, 40% yield). 1H NMR (270 MHz, CDCl₃): 8.88 (1H, s), 8.40 (1H, d), 7.62 (1H, d), 7.28-7.20 (2H, m), 4.49 (1H, q), 3.52 (1H, d), 2.05-1.78 (2H, m), 1.21 (9H, s), 0.88 (3H, t).

### Step 2

To a solution of (R)-2-methyl-propane-2-sulfinic acid {(R)-1-[4-chloro-2-fluoro-3-(thiazolo[4,5-c]pyridin-2-yloxy)-phenyl]-propyl}-amide (500 mg, 1.13 mmol, 1.0 eq) in EtOAc (40 ml) was added 2.1M HCl in EtOAc (2.12 ml, 4.45 mmol, 3.9 eq) and the reaction was stirred at RT for 1 hour then concentrated in vacuo. To a solution of the residue dissolved in EtOAc (50 ml) was added 2.1M HCl in EtOAc (1.00 ml, 2.10 mmol, 1.9 eq) and the reaction stirred for 45 min then concentrated in vacuo. The residue was slurried in 3:1 heptane:Et₂O (60 ml) for 2 hours then filtered. The solids were slurried in 1M HCl in Et₂O (3 ml) for 1 hour, filtered and washed with Et₂O (5 ml). The solids were dried in vacuo at 35° C. overnight to give (R)-1-[4-chloro-2-fluoro-3-(thiazolo[4,5-c]pyridin-2-yloxy)-phenyl]-propylamine hydrochloride (262 mg, ¹H NMR >95%, 0.700 mmol, 64% yield).

### Example 387

### (R)-1-[4-Chloro-2-fluoro-3-(5-methyl-[1,3,4]thiadiazol-2-yloxy)-phenyl]-propylamine hydrochloride

### Step 1

A flask was charged with Key Intermediate KI-3a, (R)-2-methyl-propane-2-sulfinic acid [(R)-1-(4-chloro-2-fluoro-3-hydroxy-phenyl)-propyl]-amide, (1.50 g, 4.87 mmol, 1.0 eq), 2-bromo-5-methyl-1,3,4-thiadiazole (1.31 g, 7.31 mmol, 1.5 eq), K₂CO₃ (2.69 g, 19.5 mmol, 4.0 eq) and DMF (60 ml) and the reaction was stirred under N₂ at 115° C. overnight. To the reaction was added 2-bromo-5-methyl-1,3,4-thiadiazole (0.600 g, 3.35 mmol, 0.7 eq) and stirred for a further 2 days. The reaction was allowed to cool to RT, poured into H₂O (400 ml) and extracted with 15% heptaneEtOAc (5×400 ml). The organics were washed with H₂O (5×300 ml) and brine (2×300 ml), dried over MgSO₄, filtered and concentrated in vacuo. The residue was purified via chromatography (silica, 80 g) eluting with 50% EtOAcheptanes up to 100% EtOAc. Product containing fractions combined and concentrated in vacuo to give (R)-2-methyl-propane-2-sulfinic acid {(R)-1-[4-chloro-2-fluoro-3-(5-methyl-[1,3,4]thiadiazol-2-yloxy)-phenyl]propyl}-amide (368 mg, ¹H NMR >95%, 0.951 mmol, 20% yield). 1H NMR (270 MHz, CDCl₃): 7.28-7.19 (2H, m), 4.51 (1H, q), 3.52 (1H, d), 2.66 (3H, s), 2.05-1.71 (2H, m), 1.21 (9H, s), 0.88 (3H, t).

### Step 2

To a stirred solution of (R)-2-methyl-propane-2-sulfinic acid {(R)-1-[4-chloro-2-fluoro-3-(5-methyl-[1,3,4]thiadiazol-2-yloxy)-phenyl]propyl}-amide (350 mg, 0.858 mmol, 1.0 eq) in EtOAc (30 ml) was added 2.1M HCl in EtOAc (0.41 ml, 0.858 mmol, 1.0 eq). The mixture was stirred at RT and LC indicated full conversion after 30 min. The reaction was concentrated in vacuo and the residue slurried in 3:1 heptane:Et₂O (15 ml) for 1 hour. The suspension was filtered, washed with heptanes (2×5 ml) and dried in a vacuum oven at 35° C. overnight to give (R)-1-[4-chloro-2-fluoro-3-(5-methyl-[1,3,4]thiadiazol-2-yloxy)-phenyl]-propylamine hydrochloride (167 mg, ¹H NMR >95%, 0.494 mmol, 58% yield).

### Example 388

### (R)-1-[4-Chloro-2-fluoro-3-(5-methyl-[1,3,4]oxadiazol-2-yloxy)-phenyl]-propylamine hydrochloride

### Step 1

A flask was charged with Key Intermediate KI-3a, (R)-2-methyl-propane-2-sulfinic acid [(R)-1-(4-chloro-2-fluoro-3-hydroxy-phenyl)-propyl]-amide, (1.080 g, 3.51 mmol, 1.0 eq), 2-bromo-5-methyl-1,3,4-oxadiazole (0.858 g, 5.26 mmol, 1.5 eq), K₂CO₃ (1.115 g, 8.07 mmol, 2.3 eq) and DMF (43 ml) and the reaction was stirred under N₂ at 80° C. for 16 hours. The reaction was allowed to cool to RT, poured into H₂O (200 ml) and extracted with EtOAc (2×300 ml). The organics were diluted with heptanes (100 ml) and washed with H₂O (3×200 ml) and brine (100 ml), dried over MgSO₄, filtered and concentrated in vacuo. The aqueous layers were combined and extracted with 20% MeOHEtOAc (2×300 ml) and the organic layers were combined and washed with H₂O (3×200 ml) and brine (100 ml) then dried over MgSO₄, filtered and concentrated in vacuo. The combined residues were purified via chromatography (silica, 50 g) eluting with 30% EtOAcheptanes up to 80% EtOAcheptane. Product containing fractions combined and concentrated in vacuo. The residue was dissolved in DCM (100 ml) and washed with 10% K₂CO₃ solution (100 ml), dried over MgSO₄, filtered and concentrated in vacuo. The residue was purified via chromatography (silica, 45 g), eluting with 80% heptaneEt₂O up to 100% Et₂O to give (R)-2-methyl-propane-2-sulfinic acid {(R)-1-[4-chloro-2-fluoro-3-(5-methyl-[1,3,4]oxadiazol-2-yloxy)-phenyl]-propyl}-amide (550 mg, ¹H NMR >95%, 1.41 mmol, 40% yield). 1H NMR (270 MHz, CDCl₃): 7.30-7.22 (2H, m), 4.52 (1H, q), 3.52 (1H, d), 2.49 (3H, s), 2.03-1.71 (2H, m), 1.21 (9H, s), 0.89 (3H, t).

### Step 2

To a stirred solution of (R)-2-methyl-propane-2-sulfinic acid {(R)-1-[4-chloro-2-fluoro-3-(5-methyl-[1,3,4]oxadiazol-2-yloxy)-phenyl]propyl}-amide (150 mg, 0.385 mmol, 1.0 eq) in EtOAc (6 ml) was added 2.1M HCl in EtOAc (0.18 ml, 0.39 mmol, 1.0 eq). The mixture was stirred at RT for 1 hour. 2.1M HCl in EtOAc (0.18 ml, 0.39 mmol, 1.0 eq) was added and the mixture stirred for 15 min at RT. The reaction was concentrated in vacuo and the residue slurried in heptanes (6 ml) for 60 hours. Et₂O (2 ml) was added and the mixture stirred for 1 hour then filtered, washed with heptanes (2×5 ml) and dried in a vacuum oven at 40° C. for 4 hours to give (R)-1-[4-chloro-2-fluoro-3-(5-methyl-[1,3,4]oxadiazol-2-yloxy)-phenyl]-propylamine hydrochloride (58 mg, ¹H NMR >95%, 0.18 mmol, 47% yield).

### Example 389

### {5-[3-((R)-1-Amino-propyl)-6-chloro-2-fluoro-phenoxy]-pyridin-2-yl}-dimethyl-amine hydrochloride

### Step 1

Twelve reactions were carried out: to each reaction was added Key Intermediate KI-3a, (R)-2-methyl-propane-2-sulfinic acid [(R)-1-(4-chloro-2-fluoro-3-hydroxy-phenyl)-propyl]-amide, (773 mg, 2.51 mmol, 1.0 eq), DCM (125 ml), pyridine (0.47 ml, 5.83 mmol, 2.3 eq), 2-(N,N-dimethylamino)pyridine-5-boronic acid hydrate (833 mg, 4.53 mmol, 1.8 eq) and 4 Å powdered molecular sieves (1.33 g). The mixture was stirred for 30 min before the addition of copper (II) acetate (0.57 g, 3.14 mmol, 1.25 eq). The reactions were stirred for 90 hours at room temperature before being concentrated in vacuo. To the crude material was added EtOAc (1 L) and water (1 L). The solids were filtered off, the organic layer was washed with sat. brine (2×500 ml), dried, filtered and concentrated in vacuo. The material was purified by column chromatography on silica (800 g), eluting with 100% DCM up to 50% EtOAc. The product containing fractions were combined to give 1.9 g crude material, which was dissolved in EtOAc (100 ml) and washed with 10% K₂CO₃ solution (3×30 ml). The solvent was removed and the material purified by column chromatography on silica (50 g), eluting with 100% DCM up to 30% EtOAc. The product fractions were combined to give (R)-2-methyl-propane-2-sulfinic acid {(R)-1-[4-chloro-3-(6-dimethylamino-pyridin-3-yloxy)-2-fluoro-phenyl]-propyl}-amide (530 mg, ¹H NMR >95% excluding solvents, 94% active, 1.16 mmol, 3.9% yield). 1H NMR (270 MHz, CDCl₃): 7.99 (1H, d), 7.23-7.05 (3H, m), 6.42 (1H, d), 4.44 (1H, q), 3.51 (1H, d), 3.02 (6H, s), 2.05-1.65 (2H, m), 1.20 (9H, s), 0.86 (3H, t).

### Step 2

To (R)-2-methyl-propane-2-sulfinic acid {(R)-1-[4-chloro-3-(6-dimethylamino-pyridin-3-yloxy)-2-fluoro-phenyl]-propyl}-amide (500 mg, 1.17 mmol, 1.0 eq) in EtOAc (20 ml) was added 2.1M HCl in EtOAc (1 ml, 2.1 mmol, 1.80 eq). After 1 hour, the solids were filtered off and washed with Et₂O (5 ml). Oven drying at 40° C. gave {5-[3-((R)-1-Amino-propyl)-6-chloro-2-fluoro-phenoxy]-pyridin-2-yl}-dimethyl-amine hydrochloride (446 mg, 1.24 mmol, >100% yield).

### Example 390

### 4-[3-((R)-1-Amino-propyl)-6-chloro-2-fluoro-phenoxy]-benzamide

### Step 1

To a solution of Key Intermediate KI-3e, [(R)-1-(4-chloro-2-fluoro-3-hydroxy-phenyl)-propyl]-carbamic acid tert-butyl ester (1.0 g, 3.30 mmol, 1.0 eq) in DCM (160 ml) was added 4-cyanophenyl boronic acid (0.98 g, 6.59 mmol, 2.0 eq) and powdered 4 A molecular sieves (0.8 g), followed by pyridine (0.66 ml, 7.69 mmol, 2.3 eq) and the mixture stirred until the majority was in solution. Cu(OAc)₂ (0.78 g, 4.30 mmol, 1.3 eq) was added and the mixture stirred under air for 3 days, after which time analysis (LC) indicated approximately 30% product formation. The mixture was diluted with water (160 ml), stirred for 30 minutes, then the layers separated and the aqueous extracted with DCM (100 ml). The combined organics were dried (MgSO₄), filtered and concentrated in vacuo. The crude material was purified by chromatography on silica (75 g) eluting with DCM to provide {(R)-1-[3-(4-cyano-phenoxy)-4-chloro-2-fluoro-phenyl]-propyl}-carbamic acid tert-butyl ester (0.35 g, 1H NMR 95%, 0.86 mmol, 26.2% yield). 1H NMR (270 MHz, CDCl₃): 7.63-7.58 (2H, m), 7.27-7.24 (1H, m), 7.16-7.11 (1H, m), 6.94 (2H, d), 4.89 (1H, br s), 4.76-4.68 (1H, m), 1.78-1.70 (2H, m), 1.40 (9H, br s), 0.91 (3H, t).

### Step 2

{(R)-1-[3-(4-Cyano-phenoxy)-4-chloro-2-fluoro-phenyl]-propyl}-carbamic acid tert-butyl ester (205 mg, 0.51 mmol, 1.0 eq) was suspended in ^{t}BuOH (4 ml) and heated to reflux. To the resulting solution was added KOH (85%, 85 mg, 1.29 mmol, 2.5 eq) and the mixture stirred at reflux for 5 hours, then cooled to room temperature, partitioned between DCM (30 ml) and water (50 ml). The layers separated and the aqueous extracted with DCM (3×30 ml). The combined organincs were dried (MgSO₄), filtered, concentrated in vacuo and dried overnight at 40° C. to give 4-[3-((R)-1-amino-propyl)-6-chloro-2-fluoro-phenoxy]-benzamide (95 mg, ¹H NMR >95%, 0.29 mmol, 58% yield).

### Example 391

### 5-[3-((R)-1-Amino-propyl)-6-chloro-2-fluoro-phenoxy]-pyrazine-2-carboxylic acid hydrochloride

A sample of the compound of Example 381 Step 2, (R)-5-{6-chloro-2-fluoro-3-[(R)-1-(2-methyl-propane-2-sulfinylamino)-propyl]-phenoxy}-pyrazine-2-carboxylic acid, (100 mg, 0.23 mmol, 1.0 eq) was dissolved in EtOAc (3 ml) and 2M HCl in EtOAc (1 ml, 2 mmol, 8.7 eq) was added. The resulting solid was filtered off and washed with EtOAc (1 ml) and Et₂O (1 ml) to give 5-[3-((R)-1-amino-propyl)-6-chloro-2-fluoro-phenoxy]-pyrazine-2-carboxylic acid hydrochloride (76 mg, ¹H NMR >95%, 0.21 mmol, 91% yield).

### Example 392

### 5-[3-((R)-1-Amino-propyl)-6-chloro-2-fluoro-phenoxy]-pyrazine-2-carboxylic acid amide hydrochloride

### Step 1

To a sample of the compound of Example 381 Step 2, (R)-5-{6-chloro-2-fluoro-3-[(R)-1-(2-methyl-propane-2-sulfinylamino)-propyl]-phenoxy}-pyrazine-2-carboxylic acid, (1.0 g, 2.33 mmol, 1.0 eq) in DMF (15 ml) was added NH₄Cl (1.5 g, 27.9 mmol, 12.0 eq), O-(benzotriazol-1-yl)-N,N′,N′-tetramethyluronium hexafluorophosphate (1.32 g, 3.49 mmol, 1.5 eq) and then N,N-diisopropylethylamine (3.21 ml, 18.6 mmol, 8 eq). After 16 hours at RT, the reaction was filtered and washed with DMF (5 ml). Water (200 ml) was added and extracted with EtOAc (2×200 ml). The organics were washed with sat. brine (2×50 ml), dried (MgSO₄), filtered and concentrated. The crude solid was triturated with Et₂O (15 ml), filtered and washed with Et₂O to give 5-{6-chloro-2-fluoro-3-[(R)-1-((R)-2-methyl-propane-2-sulfinylamino)-propyl]-phenoxy}-pyrazine-2-carboxylic acid amide (762 mg, ¹H NMR ˜80% active, 1.42 mmol, 61% yield). ¹H NMR (270 MHz, CDCl₃): 7.55 (1H, d), 7.32 (1H, d), 7.28-7.15 (2H, m), 4.50 (1H, q), 3.52 (1H, d), 2.05-1.72 (2H, m), 1.21 (9H, s), 0.86 (3H, t).

### Step 2

To 5-{6-chloro-2-fluoro-3-[(R)-1-((R)-2-methyl-propane-2-sulfinylamino)-propyl]-phenoxy}-pyrazine-2-carboxylic acid amide (160 mg, 0.37 mmol, 1.0 eq) in EtOAc (5 ml) was added 2M HCl in EtOAc (2 ml, 4 mmol, 10.8 eq). After 30 min, the solids were filtered off and washed with EtOAc (1 ml) and Et₂O (1 ml). The material was slurried in heptanesEt₂O (3:1, 8 ml) for 1 hour, filtered and washed with heptanes (3 ml) to give 5-[3-((R)-1-amino-propyl)-6-chloro-2-fluoro-phenoxy]-pyrazine-2-carboxylic acid amide hydro-chloride (72 mg, 0.20 mmol, 54% yield).

### Example 397

### 5-[3-((R)-Amino-cyclopropyl-methyl)-6-chloro-2-fluoro-phenoxy]-pyridin-2-ylamine hydrochloride

### Step 1

Key Intermediate KI-3b, (R)-2-methyl-propane-2-sulfinic acid [(R)-(4-chloro-2-fluoro-3-hydroxy-phenyl)-cyclopropyl-methyl]-amide, (2.0 g, 6.25 mmol, 1.0 eq), cesium carbonate (6.1 g, 18.76 mmol, 3.0 eq) and 5-chloro-2-nitropyridine (1.49 g, 9.38 mmol, 1.50 eq) in DMSO (100 ml) were heated at 50° C. for 16 hours. The mixture was poured into water (500 ml) and extracted with EtOAc (2×60 ml). The combined organic layers were washed with 10% K₂CO₃ (2×60 ml), water (60 ml) and sat. brine (60 ml) before being dried (MgSO₄), filtered and concentrated. The crude material was adsorbed onto silica (8 g) and the material purified by column chromatography on silica (50 g), eluting with 1:1 up to 2:1

EtOAc:heptanes. The combined product fractions were concentrated and stripped with diethyl ether (30 ml) to give (R)-2-methyl-propane-2-sulfinic acid {(R)-[4-chloro-2-fluoro-3-(6-nitro-pyridin-3-yloxy)-phenyl]-cyclopropyl-methyl}-amide (1.80 g, ¹H NMR >95% excluding solvent, 97% active, 3.91 mmol, 63% yield). 1H NMR (270 MHz, CDCl₃): 8.31 (1H, d), 8.26 (1H, d), 7.42-7.30 (3H, m), 3.88 (1H, dd), 3.61 (1H, d), 1.30-1.23 (1H, m), 1.21 (9H, s), 0.78-0.67 (1H, m), 0.63-0.37 (3H, m).

### Step 2

(R)-2-Methyl-propane-2-sulfinic acid {(R)-[4-chloro-2-fluoro-3-(6-nitro-pyridin-3-yloxy)-phenyl]-cyclopropyl-methyl}-amide (1.73 g, 3.91 mmol, 1.0 eq), iron powder (1094 mg, 19.6 mmol, 5.0 eq), ammonium chloride (1050 mg, 19.6 mmol, 5.0 eq) in MeOH (108 ml) and water (78 ml) was heated to reflux for 2 hours. The reaction was cooled and filtered through Celite (20 g), washing with MeOH (100 ml). The MeOH was removed in vacuo, sat. NaHCO₃ (30 ml) was added and extracted with EtOAc (60 ml). The organic layer was washed with sat. brine (20 ml), dried (MgSO₄), filtered and concentrated onto silica (6 g). The material was purified by column chromatography on silica (40 g), eluting with 100% EtOAc up to 5% MeOHEtOAc to give (R)-2-methyl-propane-2-sulfinic acid {(R)-[3-(6-amino-pyridin-3-yloxy)-4-chloro-2-fluoro-phenyl]-cyclopropyl-methyl}-amide (2001 mg, ¹H NMR >95% excluding solvent, 89% active, 4.32 mmol, 110% yield). ¹H NMR (270 MHz, CDCl₃): 7.78 (1H, d), 7.23-7.15 (2H, m), 7.10 (1H, dd), 6.45 (1H, dd), 4.27 (2H, bs), 3.84 (1H, dd), 3.58 (1H, d), 1.30-1.19 (1H, m), 1.18 (9H, s), 0.75-0.63 (1H, m), 0.59-0.35 (3H, m).

### Step 3

(R)-2-Methyl-propane-2-sulfinic acid {(R)-[3-(6-amino-pyridin-3-yloxy)-4-chloro-2-fluoro-phenyl]-cyclopropyl-methyl}-amide (1780 mg, 4.32 mmol, 1.0 eq) was dissolved in EtOAc (200 ml) at 40° C. and the solution allowed to cool to 20° C. before addition of 2.1M HClEtOAc (10 ml). After 90 min, the solvent was removed in vacuo, additional EtOAc (20 ml) was added and removed in vacuo. The solids were slurried in Et₂O (50 ml), filtered and washed with Et₂O (10 ml) to give 5-[3-((R)-amino-cyclopropyl-methyl)-6-chloro-2-fluoro-phenoxy]-pyridin-2-ylamine hydrochloride (1064 mg, ¹H NMR >95%, 3.09 mmol, 72% yield).

### Example 398

### 5-[3-((R)-Amino-cyclopropyl-methyl)-6-chloro-2-fluoro-phenoxy]-pyridine-2-carboxylic acid amide hydrochloride

### Step 1

Key Intermediate KI-3b, (R)-2-methyl-propane-2-sulfinic acid [(R)-(4-chloro-2-fluoro-3-hydroxy-phenyl)-cyclopropyl-methyl]-amide, (1.50 g, 4.69 mmol, 1.0 eq), N-methyl-2-pyrrolidone (36 ml), cesium carbonate (3.36 g, 10.32 mmol, 2.2 eq) and 5-chloro-2-cyanopyridine (715 mg, 5.16 mmol, 1.1 eq) were heated at 120° C. for 16 hours. The reaction was combined with a smaller scale reaction (500 mg). Water (300 ml) was added and extracted with EtOAc (3×60 ml). The organic layers were washed with 10% K₂CO₃ (2×60 ml), sat. brine (60 ml), dried (MgSO₄), filtered and concentrated in vacuo. The crude material was purified by column chromatography on silica (60 g), eluting with 100% heptanes then 1:1 heptanesEtOAc then 1:2 to give (R)-2-methyl-propane-2-sulfinic acid {(R)-[4-chloro-3-(6-cyano-pyridin-3-yloxy)-2-fluoro-phenyl]-cyclopropyl-methyl}-amide (1.70 g, ¹H NMR >95% excluding solvent, 83% active, 3.34 mmol, 71% yield). 1H NMR (270 MHz, CDCl₃): 8.41 (1H, d), 7.64 (1H, d), 7.38-7.26 (2H, m), 7.19 (1H, dd), 3.85 (1H, dd), 3.60 (1H, d), 1.26-1.24 (1H, m), 1.20 (9H, s), 0.77-0.65 (1H, m), 0.63-0.35 (3H, m).

### Step 2

To (R)-2-methyl-propane-2-sulfinic acid {(R)-[4-chloro-3-(6-cyano-pyridin-3-yloxy)-2-fluoro-phenyl]-cyclopropyl-methyl}-amide (1.25 g, 2.96 mmol, 1.0 eq) in THF (25 ml) was charged water (25 ml) then 2.5M NaOH (1.3 ml, 3.26 mmol, 1.1 eq). The mixture was heated at 90° C. for 16 hours before being cooled to 0° C. and extracted with TBME (3×50 ml). The organic layers were washed with sat. brine (60 ml), before being dried (MgSO₄), filtered and concentrated in vacuo. The resulting solid was slurried in Et₂O (50 ml), filtered and washed with Et₂O (20 ml) to give 5-{6-chloro-3-[(R)-cyclopropyl-((R)-2-methyl-propane-2-sulfinylamino)-methyl]-2-fluoro-phenoxy}-pyridine-2-carboxylic acid amide (807 mg, 1H NMR ˜94% [5% nitrile starting material], 1.72 mmol, 58% yield). The liquors were concentrated to give 320 mg crude material, which was taken through the reaction a second time. The crude material was purified by column chromatography on silica (10 g), eluting with 1:1 EtOAcDCM up to 100% EtOAc to provide 5-{6-chloro-3-[(R)-cyclopropyl-(2-methyl-propane-2-sulfinylamino)-methyl]-2-fluoro-phenoxy}-pyridine-2-carboxylic acid amide as a white solid (172 mg, ¹H NMR >95%, 0.39 mmol, 13% yield). 1H NMR (270 MHz, CDCl₃): 8.30 (1H, d), 8.15 (1H, d), 7.68 (1H, bs), 7.30 (2H, m), 7.20 (1H, dd), 5.48 (1H, bs), 3.85 (1H, dd), 3.60 (1H, d), 1.30-1.24 (1H, m), 1.20 (9H, s), 0.77-0.38 (4H, m).

### Step 3

To 5-{6-chloro-3-[(R)-cyclopropyl-((R)-2-methyl-propane-2-sulfinylamino)-methyl]-2-fluoro-phenoxy}-pyridine-2-carboxylic acid amide (170 mg, 0.386 mmol, 1.0 eq) in EtOAc (10 ml) was added 2.1 M HCl in EtOAc (1 ml, 2.1 mmol). After 1 hour, the solids were filtered off and washed with Et₂O (5 ml). Oven drying at 30° C. gave 5-[3-((R)-amino-cyclopropyl-methyl)-6-chloro-2-fluoro-phenoxy]-pyridine-2-carboxylic acid amide hydrochloride (128 mg, 0.344 mmol, 89% yield).

By following the methods described above, modified as necessary, the compounds listed in the Table below were prepared. In the Table, there are no Examples 47, 63, 86 and 298.

### Example 460

### 3-{[(1R)-1-{3-[(6-aminopyridin-3-yl)oxy]-4-chloro-2-fluorophenyl}propyl]amino}-3-methylbutanamide hydrochloride (1:1)

### Step 1

A solution of Key Intermediate 3 (3 g, 9.77 mmol), 5-chloro-2-nitropyridine (1.55 g, 1.17 mmol) and cesium carbonate (3.05 g, 19.5 mmol) in DMSO (24 mL) was heated to 80° C. for 2 hours. The mixture was partitioned between water and ethyl acetate and the organic fraction dried over sodium sulfate, filtered and concentrated. The residue was purified by column chromatography, eluting with 0-70% ethyl acetate in petrol to give (R)—N—[(1R)-1-{4-chloro-2-fluoro-3-[(6-nitropyridin-3-yl)oxy]phenyl}propyl]-2-methylpropane-2-sulfinamide, 375 g. MS: [M+H]⁺430.

### Step 2

A solution of (R)—N—[(1R)-1-{4-chloro-2-fluoro-3-[(6-nitropyridin-3-yl)oxy]phenyl}propyl]-2-methylpropane-2-sulfinamide (2.7 g, 6.28 mmol) in 4M HCl in 1,4-dioxane (6.28 mL) and 1,4-dioxane (31.4 mL) was stirred at room temperature for 1 hour before the mixture was concentrated. The residue was triturated with Et₂O and dried to give (1R)-1-{4-chloro-2-fluoro-3-[(6-nitropyridin-3-yl)oxy]phenyl}propan-1-amine hydrochloride as a white solid, 2.25 g, 99%. MS: [M+NH2]+ 326.

### Step 3

(1R)-1-{4-chloro-2-fluoro-3-[(6-nitropyridin-3-yl)oxy]phenyl}propan-1-amine hydrochloride (0.05 g, 0.154 mmol) was converted to the free-base by partition between CHCl₃ and saturated NaHCO₃ solution, the phases were separated and the aqueous layer was extracted into CHCl₃ (×3). Combined organic extracts were dried (Na₂SO₄), filtered and concentrated. In a screw-top vial a suspension of the residue and triethylamine hydrochloride (0.0296 g, 0.215 mmol) in 1,4-dioxane (0.261 mL) under nitrogen was stirred at 65° C. for 4 days. The mixture was concentrated diluted with EtOAc, washed with H₂O (×2), dried (Na₂SO₄), filtered and concentrated. Column chromatography eluting with a gradient of 0% EtOAcpetrol to 25% EtOAcpetrol then to 40% EtOAcpetrol gave 3-{[(1R)-1-{4-chloro-2-fluoro-3-[(6-nitropyridin-3-yl)oxy]phenyl}propyl]amino}-3-methyl-1-[(3aS,6R,7aR)-tetrahydro-8,8-dimethyl-2,2-dioxido-3H-3a,6-methano-2,1-benzisothiazol-1(4H)-yl]butan-1-one, 0.010 g, 10%. MS: [M+H]⁺ 623.2.

### Step 4

0.080 mg of 3-{[(1R)-1-{4-chloro-2-fluoro-3-[(6-nitropyridin-3-yl)oxy]phenyl}propyl]amino}-3-methyl-1-[(3aS,6R,7aR)-tetrahydro-8,8-dimethyl-2,2-dioxido-3H-3a,6-methano-2,1-benzisothiazol-1(4H)-yl]butan-1-one was treated as described in Example 277, step 2 providing 3-{[(1R)-1-{4-chloro-2-fluoro-3-[(6-nitropyridin-3-yl)oxy]phenyl}propyl]amino}-3-methylbutanoic acid which was used without further purification, MS: [M+H]⁺ 426.

### Step 5

To a stirred solution of 3-{[(1R)-1-{4-chloro-2-fluoro-3-[(6-nitropyridin-3-yl)oxy]phenyl}propyl]amino}-3-methylbutanoic acid (0.048 g, 0.113 mmol), N,N-diisopropylethylamine (0.157 mL, 0.902 mmol) and ammonium chloride (0.0301 g, 0.564 mmol) in DMF (0.676 mL) at 0° C. was added 2-(1H-7-azabenzotriazol-1-yl)-1,1,3,3-tetramethyluronium hexafluorophosphate (0.0643 g, 0.169 mmol). The mixture was allowed to warm to room temperature and stirred for 1 hour. The mixture was poured into EtOAc and washed with water (×3). The organic extracts were dried (Na₂SO₄), filtered and concentrated providing 3-{[(1R)-1-{4-chloro-2-fluoro-3-[(6-nitropyridin-3-yl)oxy]phenyl}propyl]amino}-3-methylbutanamide which was used without further purification. MS: [M+H]⁺ 425.

### Step 6

A stirred suspension of 3-{[(1R)-1-{4-chloro-2-fluoro-3-[(6-nitropyridin-3-yl)oxy]phenyl}propyl]amino}-3-methylbutanamide (0.048 g, 0.113 mmol), iron (II) sulfate heptahydrate (0.0157 g, 0.0564 mmol) and iron powder (0.0504 g, 0.902 mmol) in 1,4-dioxane (1.13 mL) and water (0.225 mL) was heated at 100° C. for 3 hours. The mixture was cooled and filtered, washing with 1,4-dioxane (×3) then DCM (×1) and concentrated. The residue was purified by preparative HPLC providing 3-{[(1R)-1-{3-[(6-aminopyridin-3-yl)oxy]-4-chloro-2-fluorophenyl}propyl]amino}-3-methylbutanamide which was converted to the hydrochloride salt, 0.016 g.

By following the methods described above, or methods analogous thereto, the compounds shown in Table A below were prepared. The numbers in the table are the example numbers.

Characterising data and details of the synthetic methods used to prepare the compounds are set out in Table B below.

TABLE A
Examples

| | |
|---|---|
| 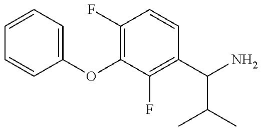 [SMILES: CC(C)C(N)c1ccc(F)c(Oc2ccccc2)c1F] | 1 |
| 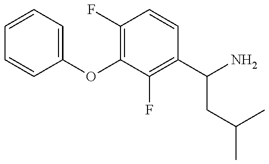 [SMILES: CC(C)CC(N)c1ccc(F)c(Oc2ccccc2)c1F] | 2 |
| 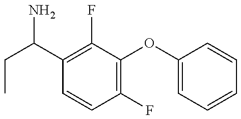 [SMILES: CCC(N)c1ccc(F)c(Oc2ccccc2)c1F] | 3 |
| 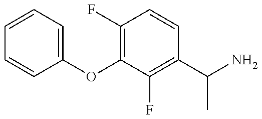 [SMILES: CC(N)c1ccc(F)c(Oc2ccccc2)c1F] | 4 |
| 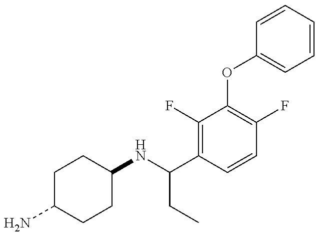 [SMILES: CCC(N[C@H]1CC[C@H](N)CC1)c1ccc(F)c(Oc2ccccc2)c1F] | 5 |
| 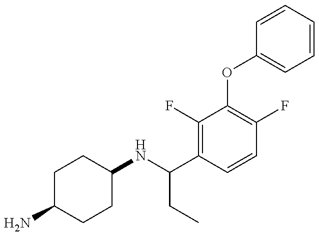 [SMILES: CCC(N[C@H]1CC[C@@H](N)CC1)c1ccc(F)c(Oc2ccccc2)c1F] | 6 |
| 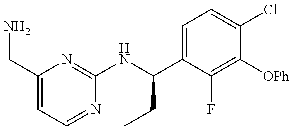 [SMILES: CC[C@@H](Nc1nccc(CN)n1)c1ccc(Cl)c(Oc2ccccc2)c1F] | 7 |
| 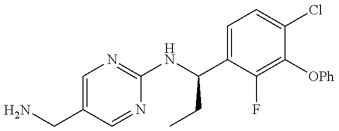 [SMILES: CC[C@@H](Nc1ncc(CN)cn1)c1ccc(Cl)c(Oc2ccccc2)c1F] | 8 |
| 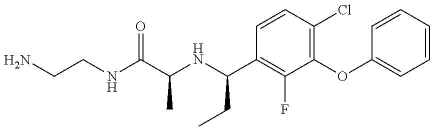 [SMILES: CC[C@@H](N[C@@H](C)C(=O)NCCN)c1ccc(Cl)c(Oc2ccccc2)c1F] | 9 |
| 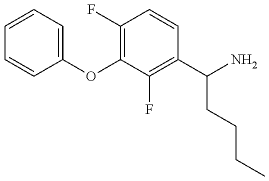 [SMILES: CCCCC(N)c1ccc(F)c(Oc2ccccc2)c1F] | 10 |
| 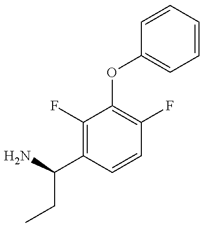 [SMILES: CC[C@@H](N)c1ccc(F)c(Oc2ccccc2)c1F] | 11 |
| 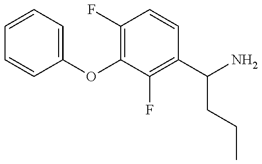 [SMILES: CCCC(N)c1ccc(F)c(Oc2ccccc2)c1F] | 12 |
| 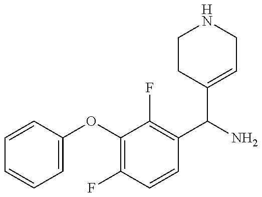 [SMILES: NC(C1=CCNCC1)c1ccc(F)c(Oc2ccccc2)c1F] | 13 |
| 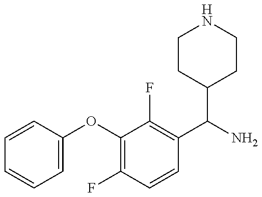 [SMILES: NC(c1ccc(F)c(Oc2ccccc2)c1F)C1CCNCC1] | 14 |
| 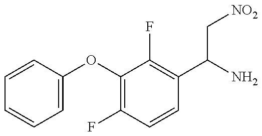 [SMILES: NC(C[N+](=O)[O-])c1ccc(F)c(Oc2ccccc2)c1F] | 15A |
| 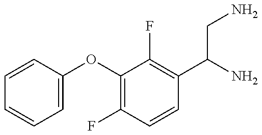 [SMILES: NCC(N)c1ccc(F)c(Oc2ccccc2)c1F] | 15B |
| 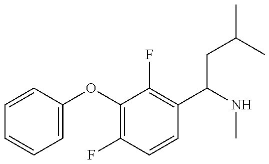 [SMILES: CNC(CC(C)C)c1ccc(F)c(Oc2ccccc2)c1F] | 16 |
| 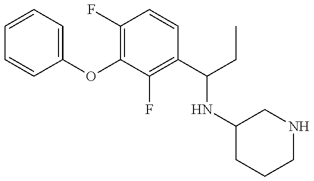 [SMILES: CCC(NC1CCCNC1)c1ccc(F)c(Oc2ccccc2)c1F] | 17 |
| 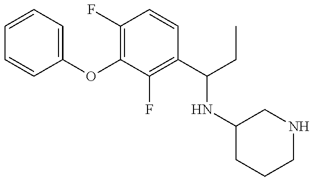 [SMILES: CCC(NC1CCCNC1)c1ccc(F)c(Oc2ccccc2)c1F] | 18 |
| 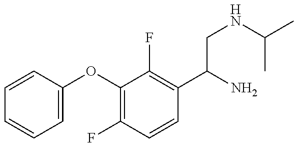 [SMILES: CC(C)NCC(N)c1ccc(F)c(Oc2ccccc2)c1F] | 19 |
| 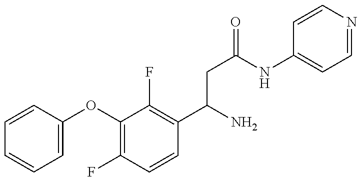 [SMILES: NC(CC(=O)Nc1ccncc1)c1ccc(F)c(Oc2ccccc2)c1F] | 20 |
| 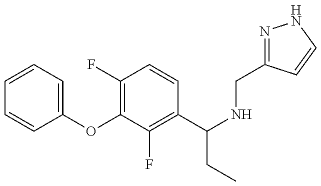 [SMILES: CCC(NCc1cc[nH]n1)c1ccc(F)c(Oc2ccccc2)c1F] | 21 |
| 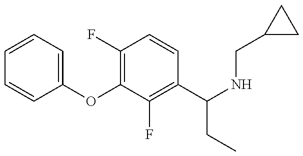 [SMILES: CCC(NCC1CC1)c1ccc(F)c(Oc2ccccc2)c1F] | 22 |
| 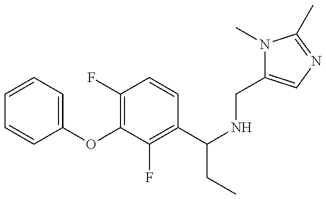 [SMILES: CCC(NCc1cnc(C)n1C)c1ccc(F)c(Oc2ccccc2)c1F] | 23 |
| 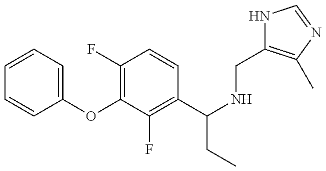 [SMILES: CCC(NCc1[nH]cnc1C)c1ccc(F)c(Oc2ccccc2)c1F] | 24 |
| 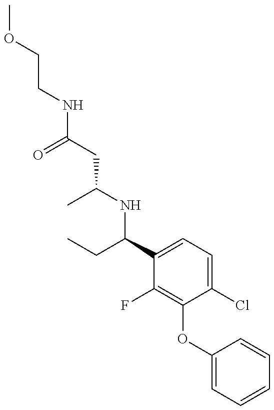 [SMILES: CC[C@@H](N[C@H](C)CC(=O)NCCOC)c1ccc(Cl)c(Oc2ccccc2)c1F] | 25 |
| 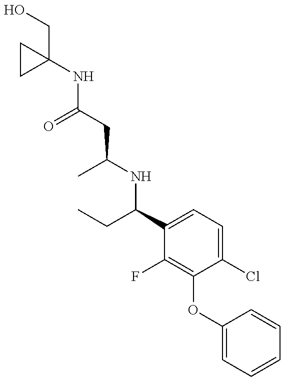 [SMILES: CC[C@@H](N[C@@H](C)CC(=O)NC1(CO)CC1)c1ccc(Cl)c(Oc2ccccc2)c1F] | 26 |
| 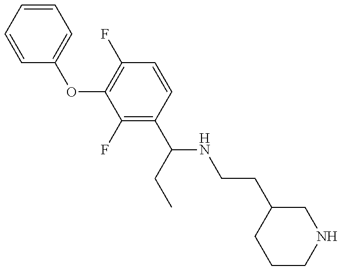 [SMILES: CCC(NCCC1CCCNC1)c1ccc(F)c(Oc2ccccc2)c1F] | 27 |
| 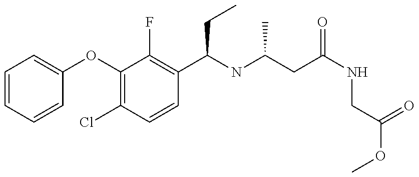 [SMILES: CC[C@@H](N[C@H](C)CC(=O)NCC(=O)OC)c1ccc(Cl)c(Oc2ccccc2)c1F] | 28 |
| 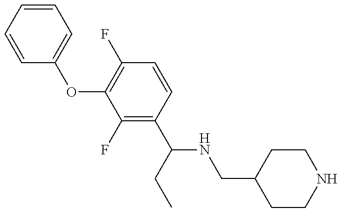 [SMILES: CCC(NCC1CCNCC1)c1ccc(F)c(Oc2ccccc2)c1F] | 29 |
| 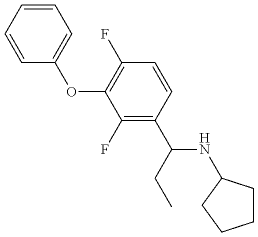 [SMILES: CCC(NC1CCCC1)c1ccc(F)c(Oc2ccccc2)c1F] | 30 |
| 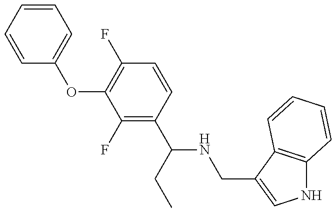 [SMILES: CCC(NCc1c[nH]c2ccccc12)c1ccc(F)c(Oc2ccccc2)c1F] | 31 |
| 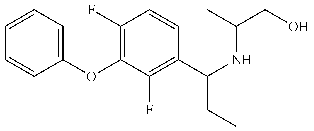 [SMILES: CCC(NC(C)CO)c1ccc(F)c(Oc2ccccc2)c1F] | 32 |
| 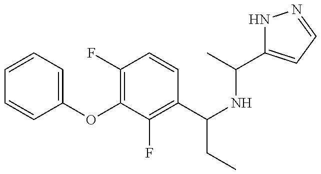 [SMILES: CCC(NC(C)c1ccn[nH]1)c1ccc(F)c(Oc2ccccc2)c1F] | 33 |
| 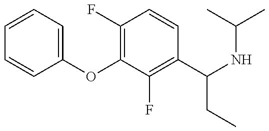 [SMILES: CCC(NC(C)C)c1ccc(F)c(Oc2ccccc2)c1F] | 34 |
| 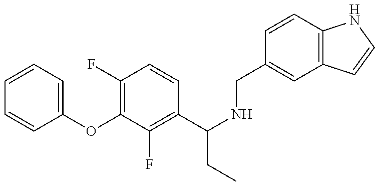 [SMILES: CCC(NCc1ccc2[nH]ccc2c1)c1ccc(F)c(Oc2ccccc2)c1F] | 35 |
| 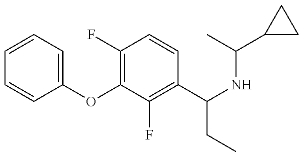 [SMILES: CCC(NC(C)C1CC1)c1ccc(F)c(Oc2ccccc2)c1F] | 36 |
| 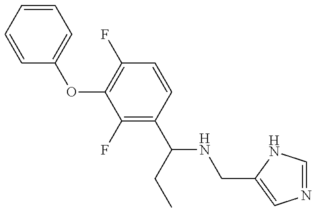 [SMILES: CCC(NCc1cnc[nH]1)c1ccc(F)c(Oc2ccccc2)c1F] | 37 |
| 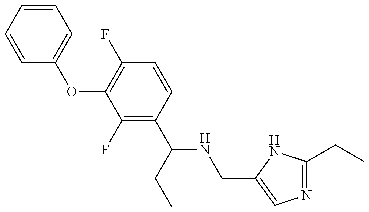 [SMILES: CCc1ncc(CNC(CC)c2ccc(F)c(Oc3ccccc3)c2F)[nH]1] | 38 |
| 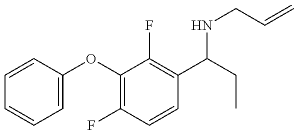 [SMILES: C=CCNC(CC)c1ccc(F)c(Oc2ccccc2)c1F] | 39 |
| 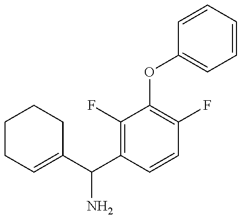 [SMILES: NC(C1=CCCCC1)c1ccc(F)c(Oc2ccccc2)c1F] | 40 |
| 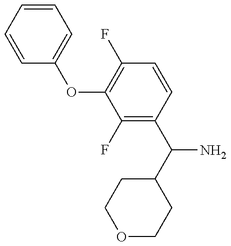 [SMILES: NC(c1ccc(F)c(Oc2ccccc2)c1F)C1CCOCC1] | 41 |
| 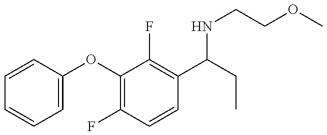 [SMILES: CCC(NCCOC)c1ccc(F)c(Oc2ccccc2)c1F] | 42 |
| 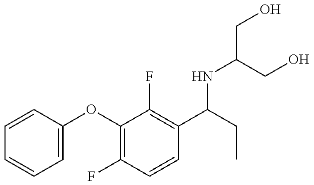 [SMILES: CCC(NC(CO)CO)c1ccc(F)c(Oc2ccccc2)c1F] | 43 |
| 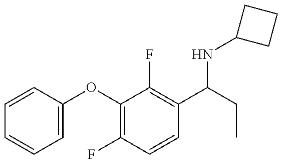 [SMILES: CCC(NC1CCC1)c1ccc(F)c(Oc2ccccc2)c1F] | 44 |
| 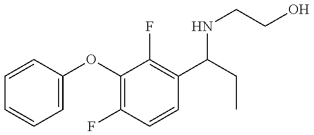 [SMILES: CCC(NCCO)c1ccc(F)c(Oc2ccccc2)c1F] | 45 |
| 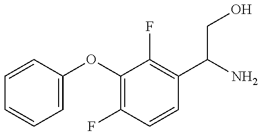 [SMILES: NC(CO)c1ccc(F)c(Oc2ccccc2)c1F] | 46 |
| 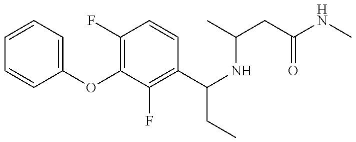 [SMILES: CCC(NC(C)CC(=O)NC)c1ccc(F)c(Oc2ccccc2)c1F] | 48 |
| 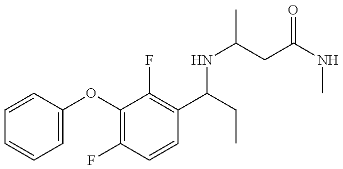 [SMILES: CCC(NC(C)CC(=O)NC)c1ccc(F)c(Oc2ccccc2)c1F] | 49 |
| 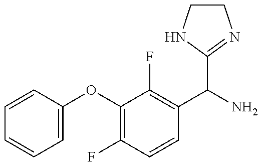 [SMILES: NC(C1=NCCN1)c1ccc(F)c(Oc2ccccc2)c1F] | 50 |
| 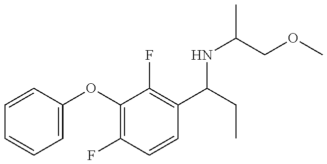 [SMILES: CCC(NC(C)COC)c1ccc(F)c(Oc2ccccc2)c1F] | 51 |
| 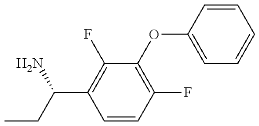 [SMILES: CC[C@H](N)c1ccc(F)c(Oc2ccccc2)c1F] | 52 |
| 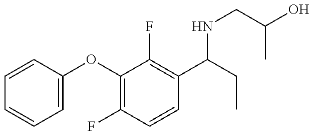 [SMILES: CCC(NCC(C)O)c1ccc(F)c(Oc2ccccc2)c1F] | 53 |
| 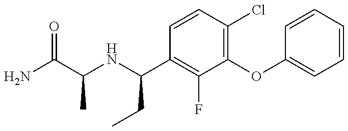 [SMILES: CC[C@@H](N[C@@H](C)C(N)=O)c1ccc(Cl)c(Oc2ccccc2)c1F] | 54 |
| 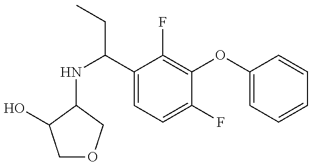 [SMILES: CCC(NC1COCC1O)c1ccc(F)c(Oc2ccccc2)c1F] | 55 |
| 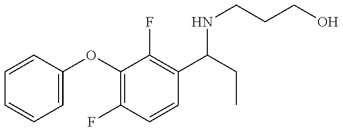 [SMILES: CCC(NCCCO)c1ccc(F)c(Oc2ccccc2)c1F] | 56 |
| 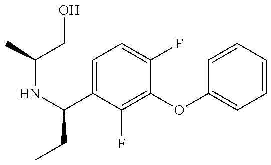 [SMILES: CC[C@@H](N[C@@H](C)CO)c1ccc(F)c(Oc2ccccc2)c1F] | 57 |
| 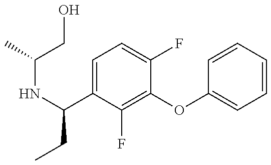 [SMILES: CC[C@@H](N[C@H](C)CO)c1ccc(F)c(Oc2ccccc2)c1F] | 58 |
| 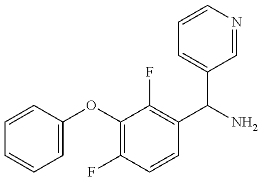 [SMILES: NC(c1cccnc1)c1ccc(F)c(Oc2ccccc2)c1F] | 59A |
| 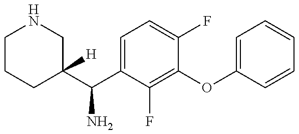 [SMILES: N[C@H](c1ccc(F)c(Oc2ccccc2)c1F)[C@@H]1CCCNC1] | 59B |
| 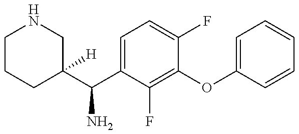 [SMILES: N[C@H](c1ccc(F)c(Oc2ccccc2)c1F)[C@H]1CCCNC1] | 60 |
| 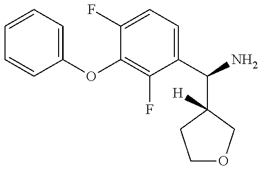 [SMILES: N[C@H](c1ccc(F)c(Oc2ccccc2)c1F)[C@H]1CCOC1] | 61 |
| 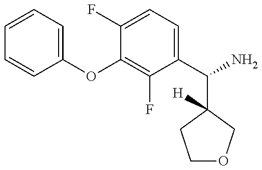 [SMILES: N[C@@H](c1ccc(F)c(Oc2ccccc2)c1F)[C@H]1CCOC1] | 62 |
| 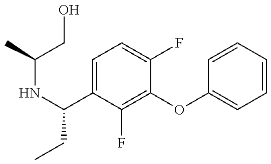 [SMILES: CC[C@H](N[C@@H](C)CO)c1ccc(F)c(Oc2ccccc2)c1F] | 64 |
| 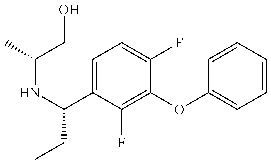 [SMILES: CC[C@H](N[C@H](C)CO)c1ccc(F)c(Oc2ccccc2)c1F] | 65 |
| 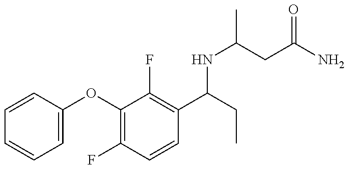 [SMILES: CCC(NC(C)CC(N)=O)c1ccc(F)c(Oc2ccccc2)c1F] | 66 |
| 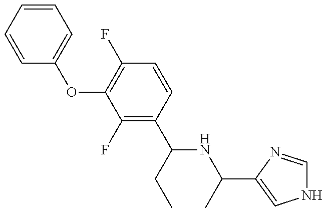 [SMILES: CCC(NC(C)c1c[nH]cn1)c1ccc(F)c(Oc2ccccc2)c1F] | 67 |
| 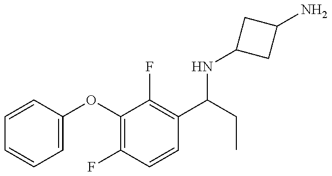 [SMILES: CCC(NC1CC(N)C1)c1ccc(F)c(Oc2ccccc2)c1F] | 68 |
| 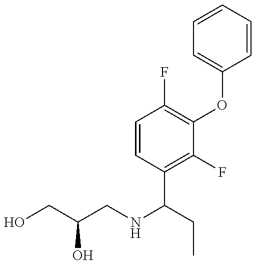 [SMILES: CCC(NC[C@@H](O)CO)c1ccc(F)c(Oc2ccccc2)c1F] | 69 |
| 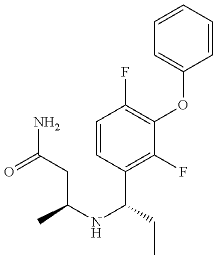 [SMILES: CC[C@H](N[C@@H](C)CC(N)=O)c1ccc(F)c(Oc2ccccc2)c1F] | 70 |
| 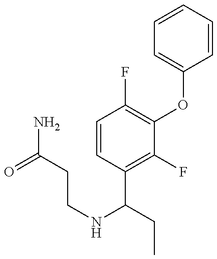 [SMILES: CCC(NCCC(N)=O)c1ccc(F)c(Oc2ccccc2)c1F] | 71 |
| 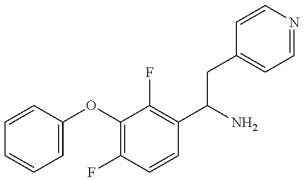 [SMILES: NC(Cc1ccncc1)c1ccc(F)c(Oc2ccccc2)c1F] | 72A |
| 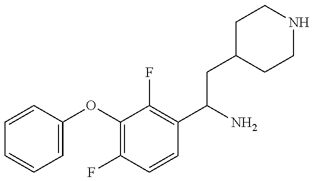 [SMILES: NC(CC1CCNCC1)c1ccc(F)c(Oc2ccccc2)c1F] | B |
| 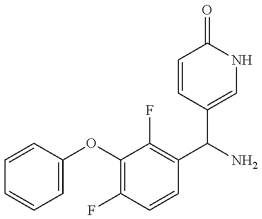 [SMILES: NC(c1ccc(=O)[nH]c1)c1ccc(F)c(Oc2ccccc2)c1F] | 73 |
| 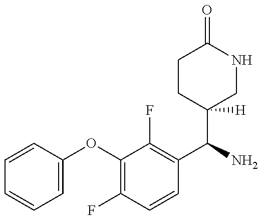 [SMILES: N[C@@H](c1ccc(F)c(Oc2ccccc2)c1F)[C@H]1CCC(=O)NC1] | 74A |
| 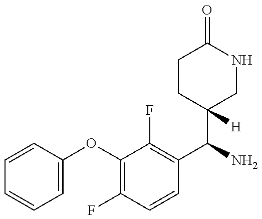 [SMILES: N[C@@H](c1ccc(F)c(Oc2ccccc2)c1F)[C@@H]1CCC(=O)NC1] | 74B |
| 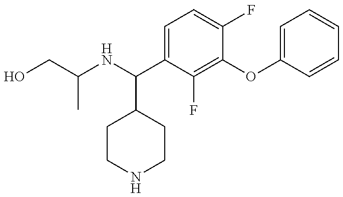 [SMILES: CC(CO)NC(c1ccc(F)c(Oc2ccccc2)c1F)C1CCNCC1] | 75 |
| 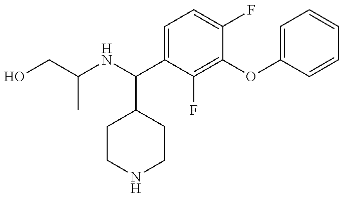 [SMILES: CC(CO)NC(c1ccc(F)c(Oc2ccccc2)c1F)C1CCNCC1] | 76 |
| 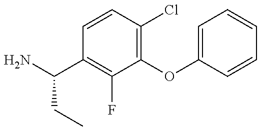 [SMILES: CC[C@H](N)c1ccc(Cl)c(Oc2ccccc2)c1F] | 77 |
| 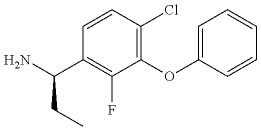 [SMILES: CC[C@@H](N)c1ccc(Cl)c(Oc2ccccc2)c1F] | 78 |
| 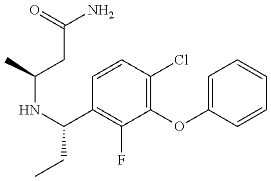 [SMILES: CC[C@H](N[C@@H](C)CC(N)=O)c1ccc(Cl)c(Oc2ccccc2)c1F] | 79 |
| 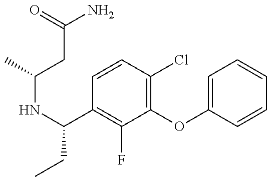 [SMILES: CC[C@H](N[C@H](C)CC(N)=O)c1ccc(Cl)c(Oc2ccccc2)c1F] | 80 |
| 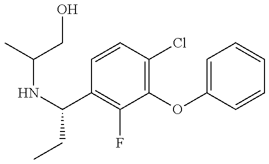 [SMILES: CC[C@H](NC(C)CO)c1ccc(Cl)c(Oc2ccccc2)c1F] | 81 |
| 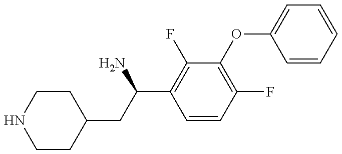 [SMILES: N[C@H](CC1CCNCC1)c1ccc(F)c(Oc2ccccc2)c1F] | 82 |
| 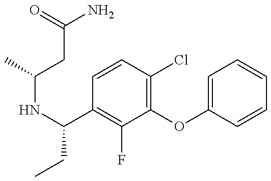 [SMILES: CC[C@H](N[C@H](C)CC(N)=O)c1ccc(Cl)c(Oc2ccccc2)c1F] | 83 |
| 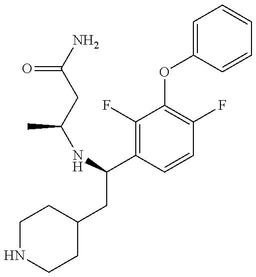 [SMILES: C[C@@H](CC(N)=O)N[C@H](CC1CCNCC1)c1ccc(F)c(Oc2ccccc2)c1F] | 84 |
| 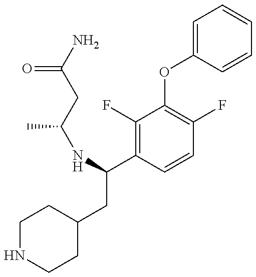 [SMILES: C[C@H](CC(N)=O)N[C@H](CC1CCNCC1)c1ccc(F)c(Oc2ccccc2)c1F] | 85 |
| 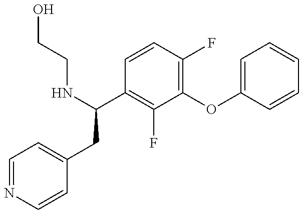 [SMILES: OCCN[C@H](Cc1ccncc1)c1ccc(F)c(Oc2ccccc2)c1F] | 87A |
| 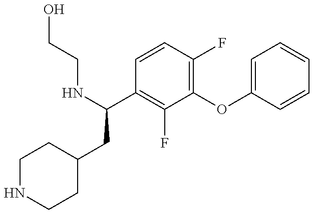 [SMILES: OCCN[C@H](CC1CCNCC1)c1ccc(F)c(Oc2ccccc2)c1F] | 87B |
| 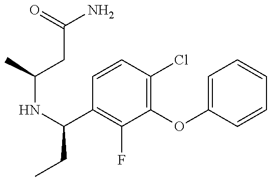 [SMILES: CC[C@@H](N[C@@H](C)CC(N)=O)c1ccc(Cl)c(Oc2ccccc2)c1F] | 88 |
| 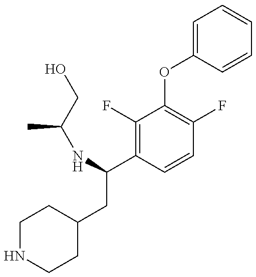 [SMILES: C[C@@H](CO)N[C@H](CC1CCNCC1)c1ccc(F)c(Oc2ccccc2)c1F] | 89 |
| 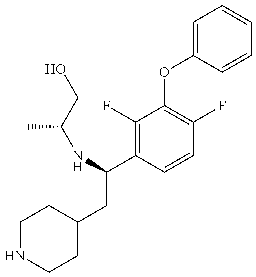 [SMILES: C[C@H](CO)N[C@H](CC1CCNCC1)c1ccc(F)c(Oc2ccccc2)c1F] | 90 |
| 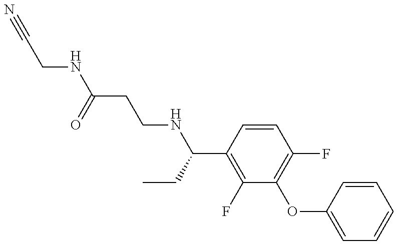 [SMILES: CC[C@H](NCCC(=O)NCC#N)c1ccc(F)c(Oc2ccccc2)c1F] | 91 |
| 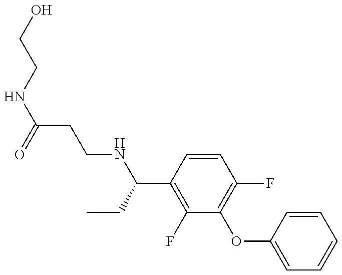 [SMILES: CC[C@H](NCCC(=O)NCCO)c1ccc(F)c(Oc2ccccc2)c1F] | 92 |
| 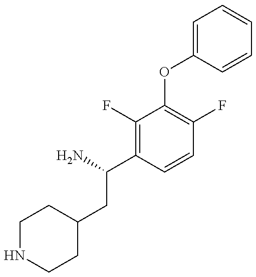 [SMILES: N[C@@H](CC1CCNCC1)c1ccc(F)c(Oc2ccccc2)c1F] | 93 |
| 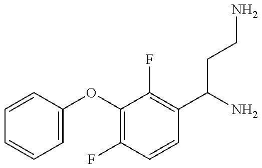 [SMILES: NCCC(N)c1ccc(F)c(Oc2ccccc2)c1F] | 94 |
| 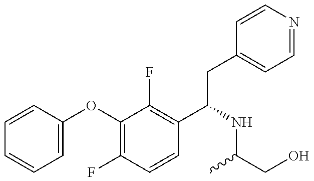 [SMILES: CC(CO)N[C@@H](Cc1ccncc1)c1ccc(F)c(Oc2ccccc2)c1F] | 95A |
| 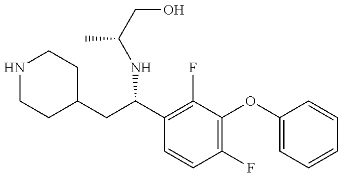 [SMILES: C[C@H](CO)N[C@@H](CC1CCNCC1)c1ccc(F)c(Oc2ccccc2)c1F] | 95B |
| 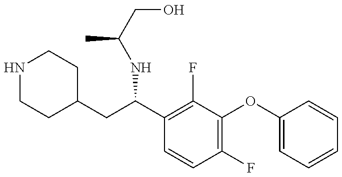 [SMILES: C[C@@H](CO)N[C@@H](CC1CCNCC1)c1ccc(F)c(Oc2ccccc2)c1F] | 96 |
| 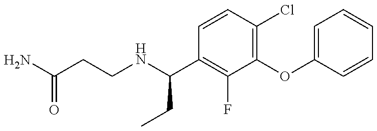 [SMILES: CC[C@@H](NCCC(N)=O)c1ccc(Cl)c(Oc2ccccc2)c1F] | 97 |
| 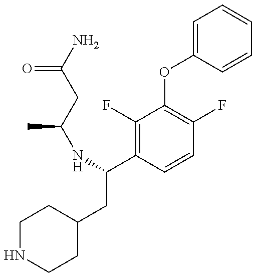 [SMILES: C[C@@H](CC(N)=O)N[C@@H](CC1CCNCC1)c1ccc(F)c(Oc2ccccc2)c1F] | 98 |
| 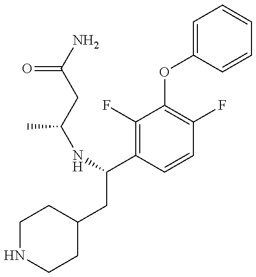 [SMILES: C[C@H](CC(N)=O)N[C@@H](CC1CCNCC1)c1ccc(F)c(Oc2ccccc2)c1F] | 99 |
| 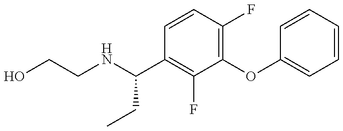 [SMILES: CC[C@H](NCCO)c1ccc(F)c(Oc2ccccc2)c1F] | 100 |
| 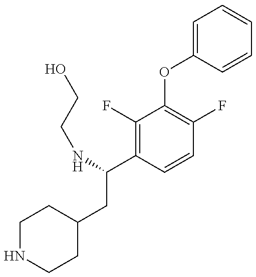 [SMILES: OCCN[C@@H](CC1CCNCC1)c1ccc(F)c(Oc2ccccc2)c1F] | 101 |
| 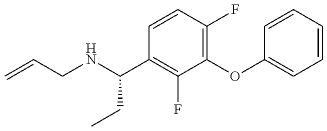 [SMILES: C=CCN[C@@H](CC)c1ccc(F)c(Oc2ccccc2)c1F] | 102 |
| 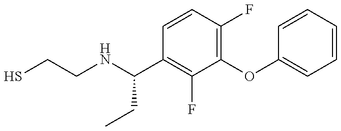 [SMILES: CC[C@H](NCCS)c1ccc(F)c(Oc2ccccc2)c1F] | 103 |
| 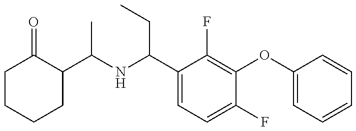 [SMILES: CCC(NC(C)C1CCCCC1=O)c1ccc(F)c(Oc2ccccc2)c1F] | 104 |
| 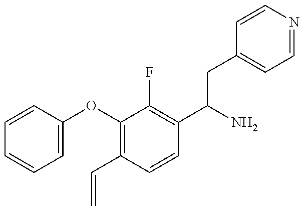 [SMILES: C=Cc1ccc(C(N)Cc2ccncc2)c(F)c1Oc1ccccc1] | 105A |
| 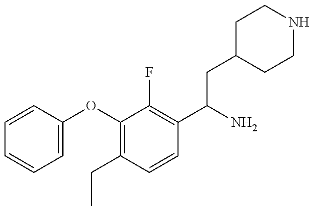 [SMILES: CCc1ccc(C(N)CC2CCNCC2)c(F)c1Oc1ccccc1] | 105B |
| 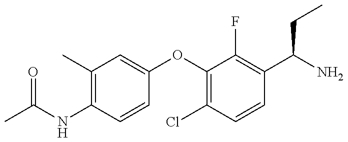 [SMILES: CC[C@@H](N)c1ccc(Cl)c(Oc2ccc(NC(C)=O)c(C)c2)c1F] | 106 |
| 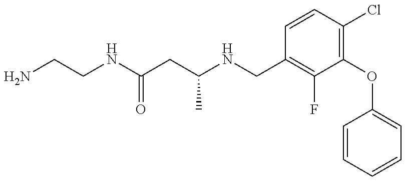 [SMILES: C[C@H](CC(=O)NCCN)NCc1ccc(Cl)c(Oc2ccccc2)c1F] | 107 |
| 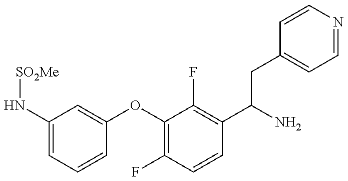 [SMILES: CS(=O)(=O)Nc1cccc(Oc2c(F)ccc(C(N)Cc3ccncc3)c2F)c1] | 108A |
| 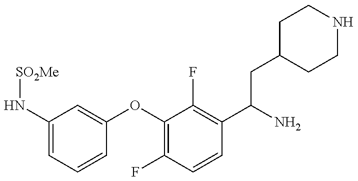 [SMILES: CS(=O)(=O)Nc1cccc(Oc2c(F)ccc(C(N)CC3CCNCC3)c2F)c1] | 108B |
| 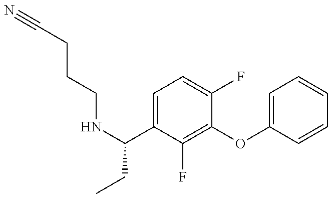 [SMILES: CC[C@H](NCCCC#N)c1ccc(F)c(Oc2ccccc2)c1F] | 109 |
| 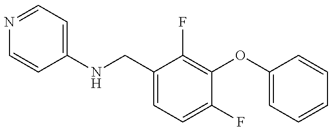 [SMILES: Fc1ccc(CNc2ccncc2)c(F)c1Oc1ccccc1] | 110 |
| 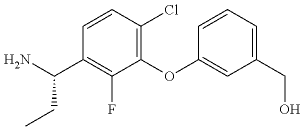 [SMILES: CC[C@H](N)c1ccc(Cl)c(Oc2cccc(CO)c2)c1F] | 111 |
| 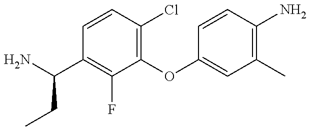 [SMILES: CC[C@@H](N)c1ccc(Cl)c(Oc2ccc(N)c(C)c2)c1F] | 112 |
| 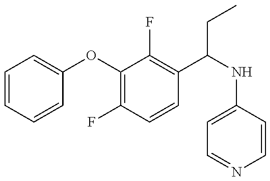 [SMILES: CCC(Nc1ccncc1)c1ccc(F)c(Oc2ccccc2)c1F] | 113 |
| 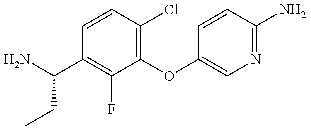 [SMILES: CC[C@H](N)c1ccc(Cl)c(Oc2ccc(N)nc2)c1F] | 114 |
| 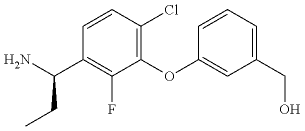 [SMILES: CC[C@@H](N)c1ccc(Cl)c(Oc2cccc(CO)c2)c1F] | 115 |
| 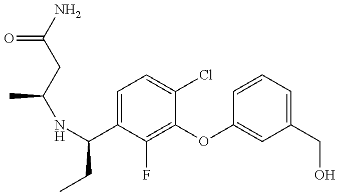 [SMILES: CC[C@@H](N[C@@H](C)CC(N)=O)c1ccc(Cl)c(Oc2cccc(CO)c2)c1F] | 116 |
| 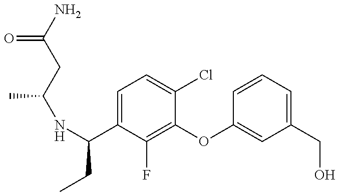 [SMILES: CC[C@@H](N[C@H](C)CC(N)=O)c1ccc(Cl)c(Oc2cccc(CO)c2)c1F] | 117 |
| 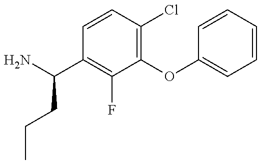 [SMILES: CCC[C@@H](N)c1ccc(Cl)c(Oc2ccccc2)c1F] | 118 |
| 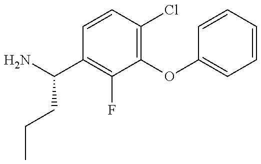 [SMILES: CCC[C@H](N)c1ccc(Cl)c(Oc2ccccc2)c1F] | 119 |
| 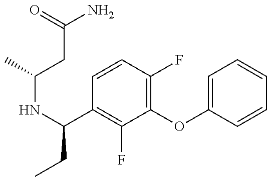 [SMILES: CC[C@@H](N[C@H](C)CC(N)=O)c1ccc(F)c(Oc2ccccc2)c1F] | 120 |
| 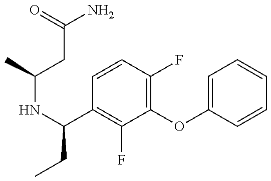 [SMILES: CC[C@@H](N[C@@H](C)CC(N)=O)c1ccc(F)c(Oc2ccccc2)c1F] | 121 |
| 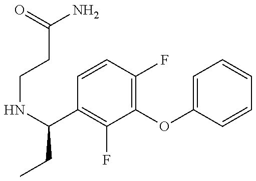 [SMILES: CC[C@@H](NCCC(N)=O)c1ccc(F)c(Oc2ccccc2)c1F] | 122 |
| 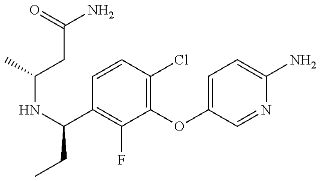 [SMILES: CC[C@@H](N[C@H](C)CC(N)=O)c1ccc(Cl)c(Oc2ccc(N)nc2)c1F] | 123 |
| 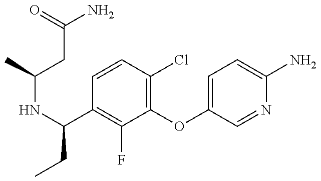 [SMILES: CC[C@@H](N[C@@H](C)CC(N)=O)c1ccc(Cl)c(Oc2ccc(N)nc2)c1F] | 124 |
| 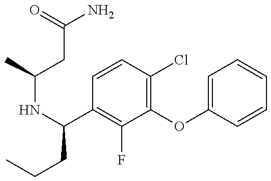 [SMILES: CCC[C@@H](N[C@@H](C)CC(N)=O)c1ccc(Cl)c(Oc2ccccc2)c1F] | 125 |
| 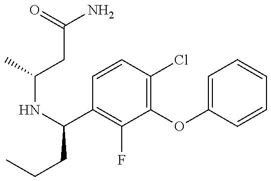 [SMILES: CCC[C@@H](N[C@H](C)CC(N)=O)c1ccc(Cl)c(Oc2ccccc2)c1F] | 126 |
| 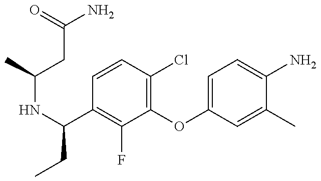 [SMILES: CC[C@@H](N[C@@H](C)CC(N)=O)c1ccc(Cl)c(Oc2ccc(N)c(C)c2)c1F] | 127 |
| 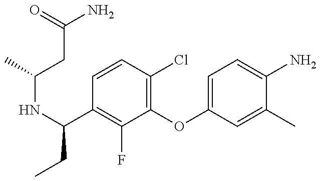 [SMILES: CC[C@@H](N[C@H](C)CC(N)=O)c1ccc(Cl)c(Oc2ccc(N)c(C)c2)c1F] | 128 |
| 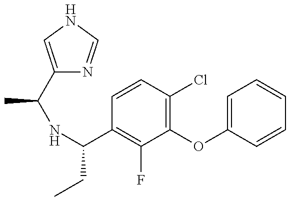 [SMILES: CC[C@H](N[C@@H](C)c1c[nH]cn1)c1ccc(Cl)c(Oc2ccccc2)c1F] | 129 |
| 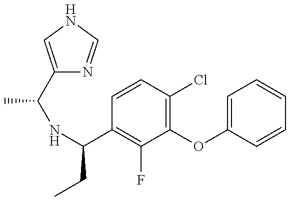 [SMILES: CC[C@@H](N[C@H](C)c1c[nH]cn1)c1ccc(Cl)c(Oc2ccccc2)c1F] | 130 |
| 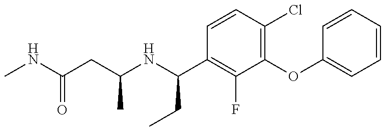 [SMILES: CC[C@@H](N[C@@H](C)CC(=O)NC)c1ccc(Cl)c(Oc2ccccc2)c1F] | 131A |
| 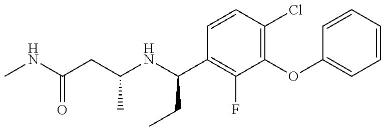 [SMILES: CC[C@@H](N[C@H](C)CC(=O)NC)c1ccc(Cl)c(Oc2ccccc2)c1F] | 131B |
| 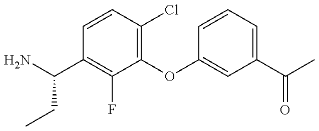 [SMILES: CC[C@H](N)c1ccc(Cl)c(Oc2cccc(C(C)=O)c2)c1F] | 132A |
| 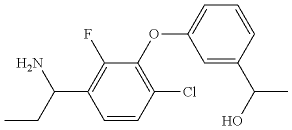 [SMILES: CCC(N)c1ccc(Cl)c(Oc2cccc(C(C)O)c2)c1F] | 132B |
| 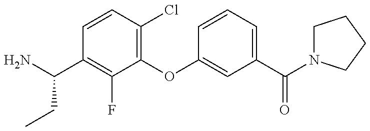 [SMILES: CC[C@H](N)c1ccc(Cl)c(Oc2cccc(C(=O)N3CCCC3)c2)c1F] | 133 |
| 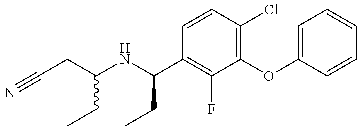 [SMILES: CCC(CC#N)N[C@H](CC)c1ccc(Cl)c(Oc2ccccc2)c1F] | 134A |
| 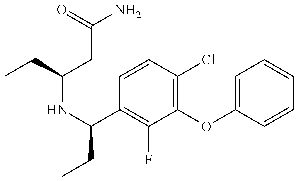 [SMILES: CC[C@@H](CC(N)=O)N[C@H](CC)c1ccc(Cl)c(Oc2ccccc2)c1F] | 134B |
| 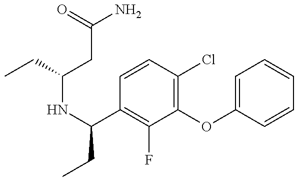 [SMILES: CC[C@H](CC(N)=O)N[C@H](CC)c1ccc(Cl)c(Oc2ccccc2)c1F] | 135 |
| 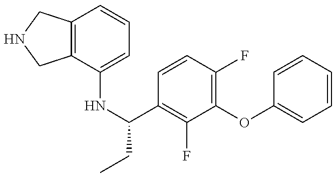 [SMILES: CC[C@H](Nc1cccc2c1CNC2)c1ccc(F)c(Oc2ccccc2)c1F] | 136 |
| 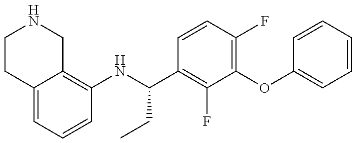 [SMILES: CC[C@H](Nc1cccc2c1CNCC2)c1ccc(F)c(Oc2ccccc2)c1F] | 137 |
| 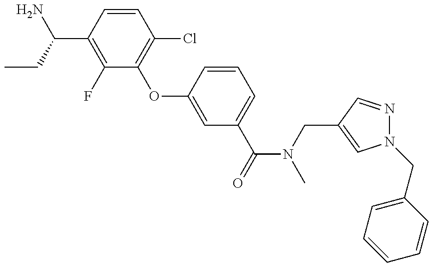 [SMILES: CC[C@H](N)c1ccc(Cl)c(Oc2cccc(C(=O)N(C)Cc3cnn(Cc4ccccc4)c3)c2)c1F] | 138 |
| 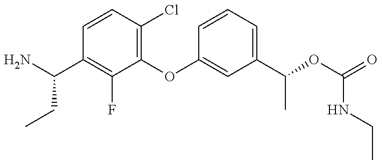 [SMILES: CCNC(=O)O[C@H](C)c1cccc(Oc2c(Cl)ccc([C@@H](N)CC)c2F)c1] | 139 |
| 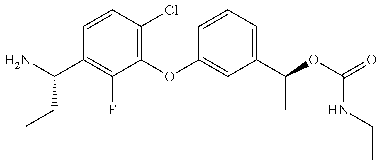 [SMILES: CCNC(=O)O[C@@H](C)c1cccc(Oc2c(Cl)ccc([C@@H](N)CC)c2F)c1] | 140 |
| 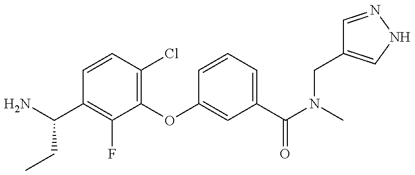 [SMILES: CC[C@H](N)c1ccc(Cl)c(Oc2cccc(C(=O)N(C)Cc3cn[nH]c3)c2)c1F] | 141 |
| 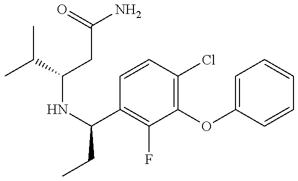 [SMILES: CC[C@@H](N[C@@H](CC(N)=O)C(C)C)c1ccc(Cl)c(Oc2ccccc2)c1F] | 142 |
| 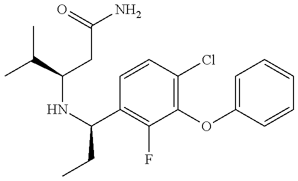 [SMILES: CC[C@@H](N[C@H](CC(N)=O)C(C)C)c1ccc(Cl)c(Oc2ccccc2)c1F] | 143 |
| 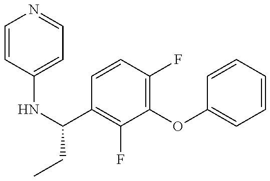 [SMILES: CC[C@H](Nc1ccncc1)c1ccc(F)c(Oc2ccccc2)c1F] | 144 |
| 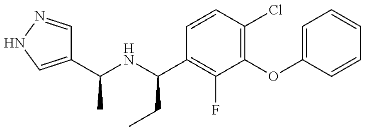 [SMILES: CC[C@@H](N[C@@H](C)c1cn[nH]c1)c1ccc(Cl)c(Oc2ccccc2)c1F] | 145A |
| 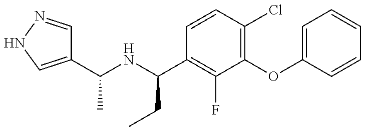 [SMILES: CC[C@@H](N[C@H](C)c1cn[nH]c1)c1ccc(Cl)c(Oc2ccccc2)c1F] | 145B |
| 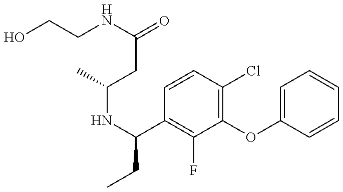 [SMILES: CC[C@@H](N[C@H](C)CC(=O)NCCO)c1ccc(Cl)c(Oc2ccccc2)c1F] | 146 |
| 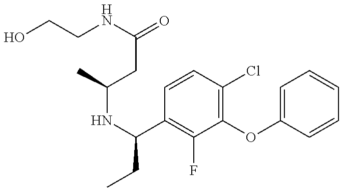 [SMILES: CC[C@@H](N[C@@H](C)CC(=O)NCCO)c1ccc(Cl)c(Oc2ccccc2)c1F] | 147 |
| 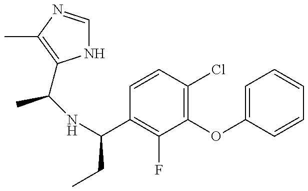 [SMILES: CC[C@@H](N[C@@H](C)c1[nH]cnc1C)c1ccc(Cl)c(Oc2ccccc2)c1F] | 148 |
| 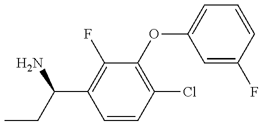 [SMILES: CC[C@@H](N)c1ccc(Cl)c(Oc2cccc(F)c2)c1F] | 149 |
| 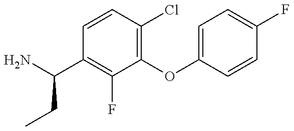 [SMILES: CC[C@@H](N)c1ccc(Cl)c(Oc2ccc(F)cc2)c1F] | 150 |
| 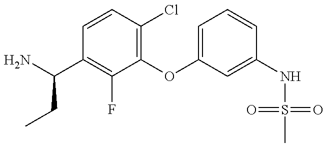 [SMILES: CC[C@@H](N)c1ccc(Cl)c(Oc2cccc(NS(C)(=O)=O)c2)c1F] | 151 |
| 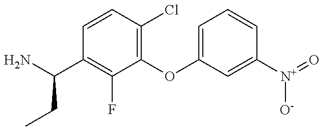 [SMILES: CC[C@@H](N)c1ccc(Cl)c(Oc2cccc([N+](=O)[O-])c2)c1F] | 152 |
| 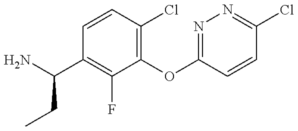 [SMILES: CC[C@@H](N)c1ccc(Cl)c(Oc2ccc(Cl)nn2)c1F] | 153 |
| 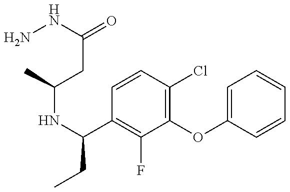 [SMILES: CC[C@@H](N[C@@H](C)CC(=O)NN)c1ccc(Cl)c(Oc2ccccc2)c1F] | 154 |
| 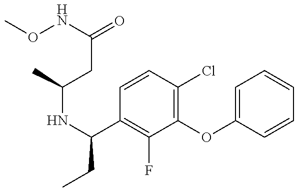 [SMILES: CC[C@@H](N[C@@H](C)CC(=O)NOC)c1ccc(Cl)c(Oc2ccccc2)c1F] | 155 |
| 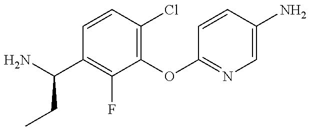 [SMILES: CC[C@@H](N)c1ccc(Cl)c(Oc2ccc(N)cn2)c1F] | 156 |
| 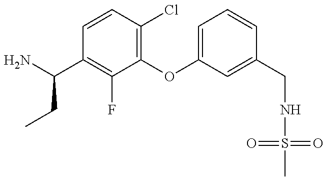 [SMILES: CC[C@@H](N)c1ccc(Cl)c(Oc2cccc(CNS(C)(=O)=O)c2)c1F] | 157 |
| 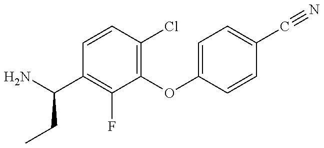 [SMILES: CC[C@@H](N)c1ccc(Cl)c(Oc2ccc(C#N)cc2)c1F] | 158 |
| 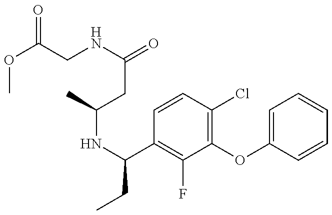 [SMILES: CC[C@@H](N[C@@H](C)CC(=O)NCC(=O)OC)c1ccc(Cl)c(Oc2ccccc2)c1F] | 159 |
| 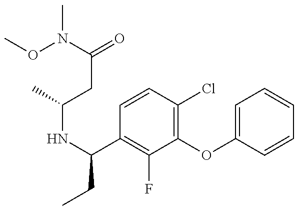 [SMILES: CC[C@@H](N[C@H](C)CC(=O)N(C)OC)c1ccc(Cl)c(Oc2ccccc2)c1F] | 160 |
| 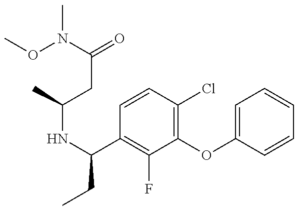 [SMILES: CC[C@@H](N[C@@H](C)CC(=O)N(C)OC)c1ccc(Cl)c(Oc2ccccc2)c1F] | 161 |
| 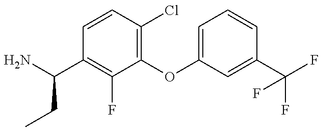 [SMILES: CC[C@@H](N)c1ccc(Cl)c(Oc2cccc(C(F)(F)F)c2)c1F] | 162 |
| 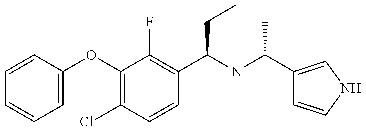 [SMILES: CC[C@@H](N[C@H](C)c1cc[nH]c1)c1ccc(Cl)c(Oc2ccccc2)c1F] | 163 |
| 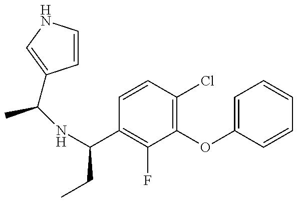 [SMILES: CC[C@@H](N[C@@H](C)c1cc[nH]c1)c1ccc(Cl)c(Oc2ccccc2)c1F] | 164 |
| 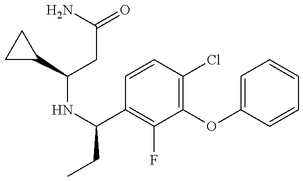 [SMILES: CC[C@@H](N[C@H](CC(N)=O)C1CC1)c1ccc(Cl)c(Oc2ccccc2)c1F] | 165 |
| 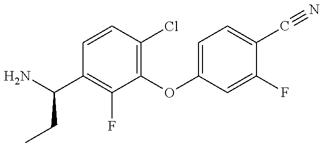 [SMILES: CC[C@@H](N)c1ccc(Cl)c(Oc2ccc(C#N)c(F)c2)c1F] | 166 |
| 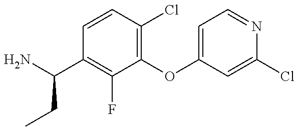 [SMILES: CC[C@@H](N)c1ccc(Cl)c(Oc2ccnc(Cl)c2)c1F] | 167 |
| 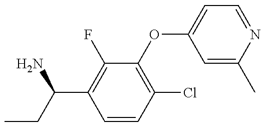 [SMILES: CC[C@@H](N)c1ccc(Cl)c(Oc2ccnc(C)c2)c1F] | 168 |
| 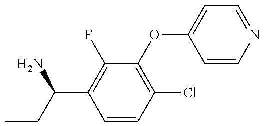 [SMILES: CC[C@@H](N)c1ccc(Cl)c(Oc2ccncc2)c1F] | 169 |
| 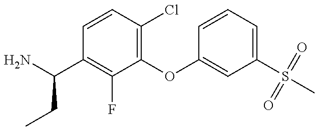 [SMILES: CC[C@@H](N)c1ccc(Cl)c(Oc2cccc(S(C)(=O)=O)c2)c1F] | 170 |
| 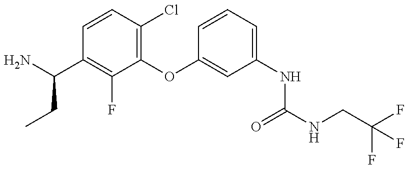 [SMILES: CC[C@@H](N)c1ccc(Cl)c(Oc2cccc(NC(=O)NCC(F)(F)F)c2)c1F] | 171 |
| 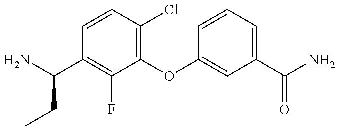 [SMILES: CC[C@@H](N)c1ccc(Cl)c(Oc2cccc(C(N)=O)c2)c1F] | 172 |
| 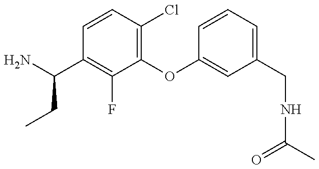 [SMILES: CC[C@@H](N)c1ccc(Cl)c(Oc2cccc(CNC(C)=O)c2)c1F] | 173 |
| 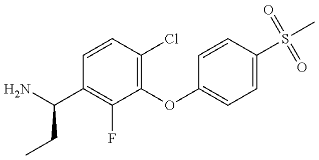 [SMILES: CC[C@@H](N)c1ccc(Cl)c(Oc2ccc(S(C)(=O)=O)cc2)c1F] | 174 |
| 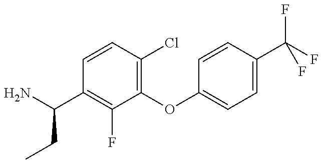 [SMILES: CC[C@@H](N)c1ccc(Cl)c(Oc2ccc(C(F)(F)F)cc2)c1F] | 175 |
| 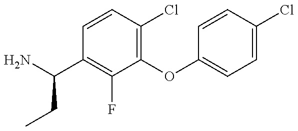 [SMILES: CC[C@@H](N)c1ccc(Cl)c(Oc2ccc(Cl)cc2)c1F] | 176 |
| 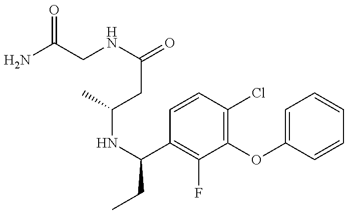 [SMILES: CC[C@@H](N[C@H](C)CC(=O)NCC(N)=O)c1ccc(Cl)c(Oc2ccccc2)c1F] | 177 |
| 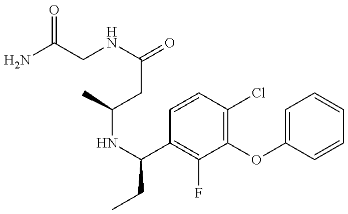 [SMILES: CC[C@@H](N[C@@H](C)CC(=O)NCC(N)=O)c1ccc(Cl)c(Oc2ccccc2)c1F] | 178 |
| 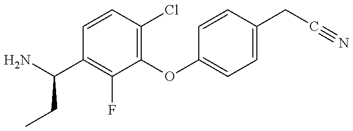 [SMILES: CC[C@@H](N)c1ccc(Cl)c(Oc2ccc(CC#N)cc2)c1F] | 179 |
| 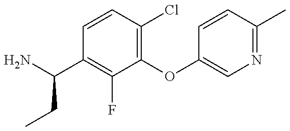 [SMILES: CC[C@@H](N)c1ccc(Cl)c(Oc2ccc(C)nc2)c1F] | 180 |
| 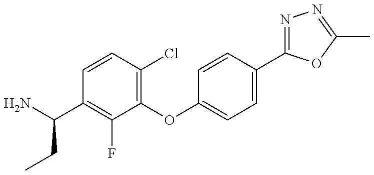 [SMILES: CC[C@@H](N)c1ccc(Cl)c(Oc2ccc(-c3nnc(C)o3)cc2)c1F] | 181 |
| 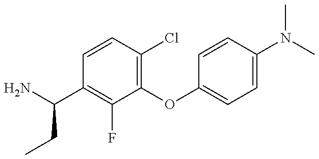 [SMILES: CC[C@@H](N)c1ccc(Cl)c(Oc2ccc(N(C)C)cc2)c1F] | 182 |
| 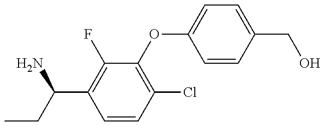 [SMILES: CC[C@@H](N)c1ccc(Cl)c(Oc2ccc(CO)cc2)c1F] | 183 |
| 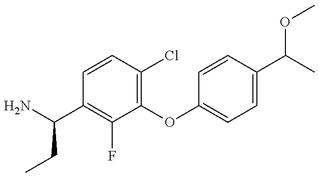 [SMILES: CC[C@@H](N)c1ccc(Cl)c(Oc2ccc(C(C)OC)cc2)c1F] | 184 |
| 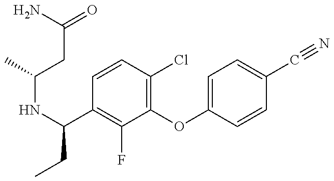 [SMILES: CC[C@@H](N[C@H](C)CC(N)=O)c1ccc(Cl)c(Oc2ccc(C#N)cc2)c1F] | 185 |
| 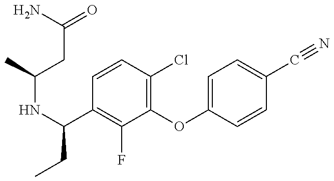 [SMILES: CC[C@@H](N[C@@H](C)CC(N)=O)c1ccc(Cl)c(Oc2ccc(C#N)cc2)c1F] | 186 |
| 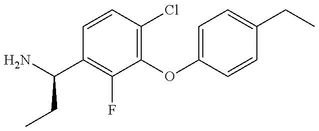 [SMILES: CCc1ccc(Oc2c(Cl)ccc([C@H](N)CC)c2F)cc1] | 187 |
| 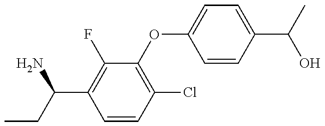 [SMILES: CC[C@@H](N)c1ccc(Cl)c(Oc2ccc(C(C)O)cc2)c1F] | 188 |
| 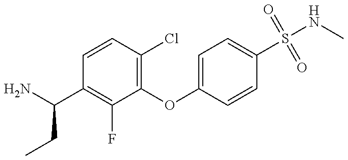 [SMILES: CC[C@@H](N)c1ccc(Cl)c(Oc2ccc(S(=O)(=O)NC)cc2)c1F] | 189 |
| 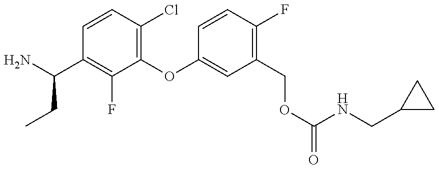 [SMILES: CC[C@@H](N)c1ccc(Cl)c(Oc2ccc(F)c(COC(=O)NCC3CC3)c2)c1F] | 190 |
| 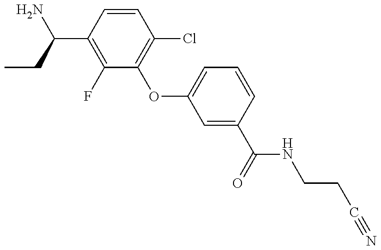 [SMILES: CC[C@@H](N)c1ccc(Cl)c(Oc2cccc(C(=O)NCCC#N)c2)c1F] | 191 |
| 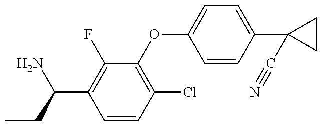 [SMILES: CC[C@@H](N)c1ccc(Cl)c(Oc2ccc(C3(C#N)CC3)cc2)c1F] | 192 |
| 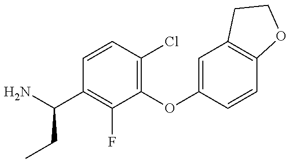 [SMILES: CC[C@@H](N)c1ccc(Cl)c(Oc2ccc3c(c2)CCO3)c1F] | 193 |
| 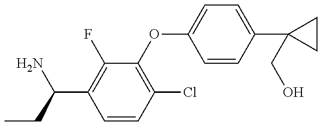 [SMILES: CC[C@@H](N)c1ccc(Cl)c(Oc2ccc(C3(CO)CC3)cc2)c1F] | 194 |
| 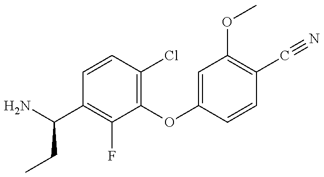 [SMILES: CC[C@@H](N)c1ccc(Cl)c(Oc2ccc(C#N)c(OC)c2)c1F] | 195 |
| 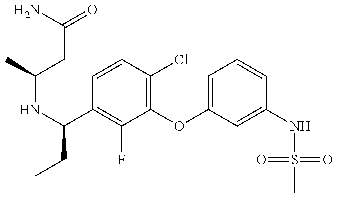 [SMILES: CC[C@@H](N[C@@H](C)CC(N)=O)c1ccc(Cl)c(Oc2cccc(NS(C)(=O)=O)c2)c1F] | 196 |
| 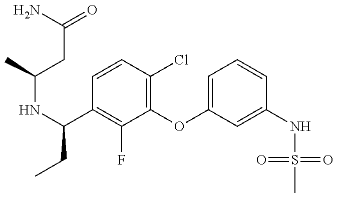 [SMILES: CC[C@@H](N[C@@H](C)CC(N)=O)c1ccc(Cl)c(Oc2cccc(NS(C)(=O)=O)c2)c1F] | 196 |
| 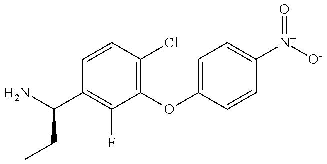 [SMILES: CC[C@@H](N)c1ccc(Cl)c(Oc2ccc([N+](=O)[O-])cc2)c1F] | 197 |
| 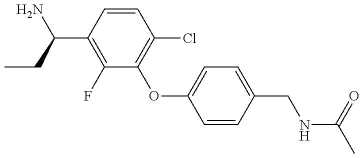 [SMILES: CC[C@@H](N)c1ccc(Cl)c(Oc2ccc(CNC(C)=O)cc2)c1F] | 198 |
| 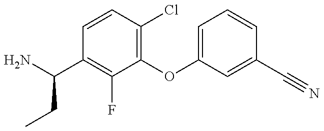 [SMILES: CC[C@@H](N)c1ccc(Cl)c(Oc2cccc(C#N)c2)c1F] | 199 |
| 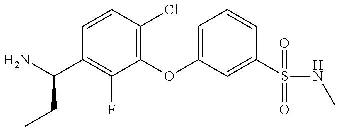 [SMILES: CC[C@@H](N)c1ccc(Cl)c(Oc2cccc(S(=O)(=O)NC)c2)c1F] | 200 |
| 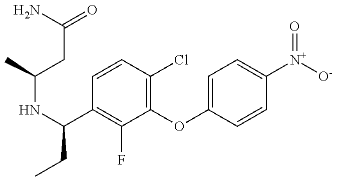 [SMILES: CC[C@@H](N[C@@H](C)CC(N)=O)c1ccc(Cl)c(Oc2ccc([N+](=O)[O-])cc2)c1F] | 201 |
| 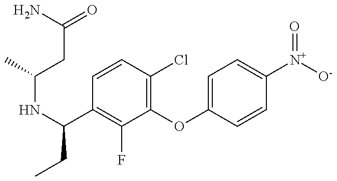 [SMILES: CC[C@@H](N[C@H](C)CC(N)=O)c1ccc(Cl)c(Oc2ccc([N+](=O)[O-])cc2)c1F] | 202 |
| 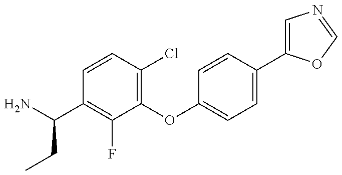 [SMILES: CC[C@@H](N)c1ccc(Cl)c(Oc2ccc(-c3cnco3)cc2)c1F] | 203 |
| 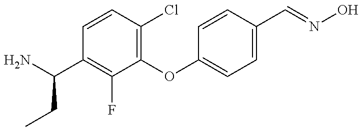 [SMILES: CC[C@@H](N)c1ccc(Cl)c(Oc2ccc(/C=N/O)cc2)c1F] | 204 |
| 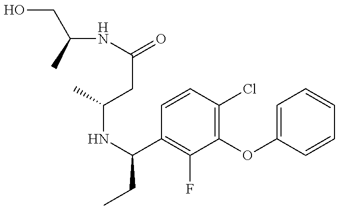 [SMILES: CC[C@@H](N[C@H](C)CC(=O)N[C@@H](C)CO)c1ccc(Cl)c(Oc2ccccc2)c1F] | 205 |
| 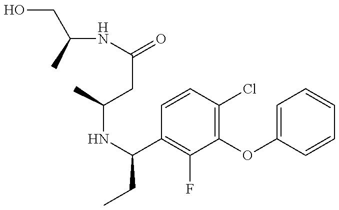 [SMILES: CC[C@@H](N[C@@H](C)CC(=O)N[C@@H](C)CO)c1ccc(Cl)c(Oc2ccccc2)c1F] | 206 |
| 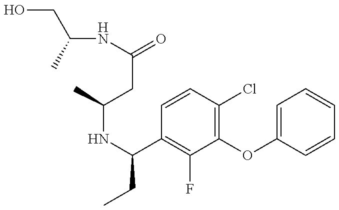 [SMILES: CC[C@@H](N[C@@H](C)CC(=O)N[C@H](C)CO)c1ccc(Cl)c(Oc2ccccc2)c1F] | 207 |
| 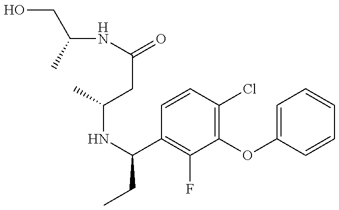 [SMILES: CC[C@@H](N[C@H](C)CC(=O)N[C@H](C)CO)c1ccc(Cl)c(Oc2ccccc2)c1F] | 208 |
| 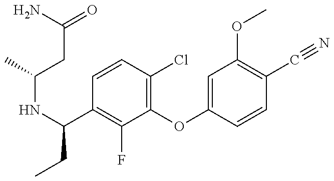 [SMILES: CC[C@@H](N[C@H](C)CC(N)=O)c1ccc(Cl)c(Oc2ccc(C#N)c(OC)c2)c1F] | 209 |
| 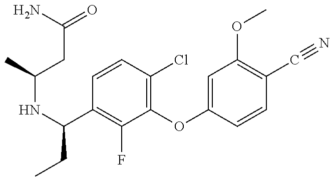 [SMILES: CC[C@@H](N[C@@H](C)CC(N)=O)c1ccc(Cl)c(Oc2ccc(C#N)c(OC)c2)c1F] | 210 |
| 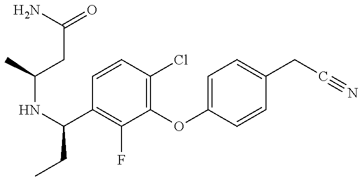 [SMILES: CC[C@@H](N[C@@H](C)CC(N)=O)c1ccc(Cl)c(Oc2ccc(CC#N)cc2)c1F] | 211 |
| 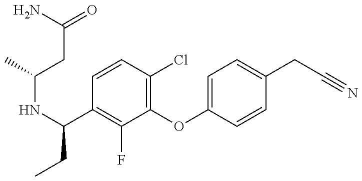 [SMILES: CC[C@@H](N[C@H](C)CC(N)=O)c1ccc(Cl)c(Oc2ccc(CC#N)cc2)c1F] | 212 |
| 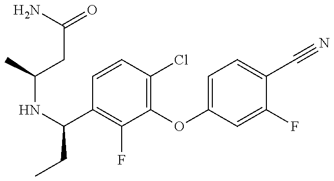 [SMILES: CC[C@@H](N[C@@H](C)CC(N)=O)c1ccc(Cl)c(Oc2ccc(C#N)c(F)c2)c1F] | 213 |
| 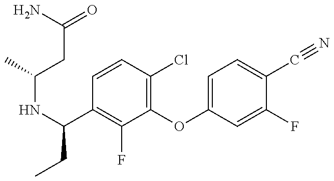 [SMILES: CC[C@@H](N[C@H](C)CC(N)=O)c1ccc(Cl)c(Oc2ccc(C#N)c(F)c2)c1F] | 214 |
| 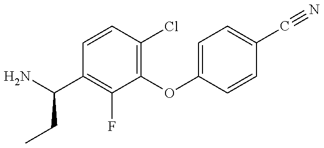 [SMILES: CC[C@@H](N)c1ccc(Cl)c(Oc2ccc(C#N)cc2)c1F] | 215 |
| 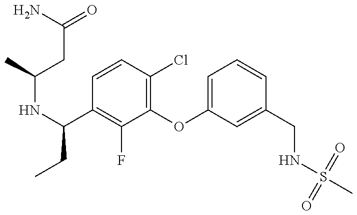 [SMILES: CC[C@@H](N[C@@H](C)CC(N)=O)c1ccc(Cl)c(Oc2cccc(CNS(C)(=O)=O)c2)c1F] | 216 |
| 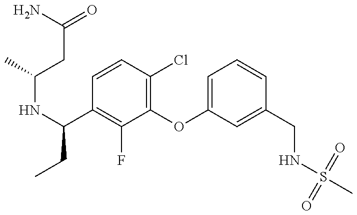 [SMILES: CC[C@@H](N[C@H](C)CC(N)=O)c1ccc(Cl)c(Oc2cccc(CNS(C)(=O)=O)c2)c1F] | 217 |
| 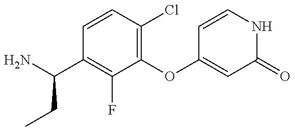 [SMILES: CC[C@@H](N)c1ccc(Cl)c(Oc2cc[nH]c(=O)c2)c1F] | 218 |
| 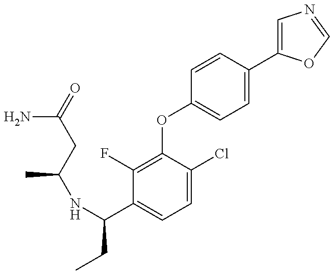 [SMILES: CC[C@@H](N[C@@H](C)CC(N)=O)c1ccc(Cl)c(Oc2ccc(-c3cnco3)cc2)c1F] | 219 |
| 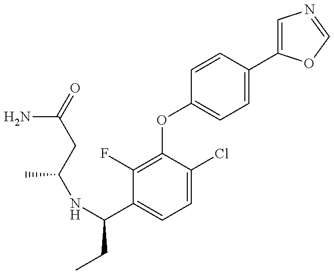 [SMILES: CC[C@@H](N[C@H](C)CC(N)=O)c1ccc(Cl)c(Oc2ccc(-c3cnco3)cc2)c1F] | 220 |
| 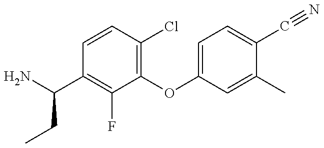 [SMILES: CC[C@@H](N)c1ccc(Cl)c(Oc2ccc(C#N)c(C)c2)c1F] | 221 |
| 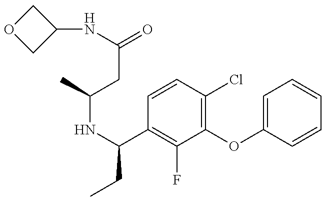 [SMILES: CC[C@@H](N[C@@H](C)CC(=O)NC1COC1)c1ccc(Cl)c(Oc2ccccc2)c1F] | 222 |
| 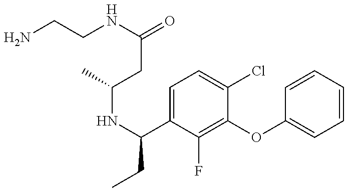 [SMILES: CC[C@@H](N[C@H](C)CC(=O)NCCN)c1ccc(Cl)c(Oc2ccccc2)c1F] | 223 |
| 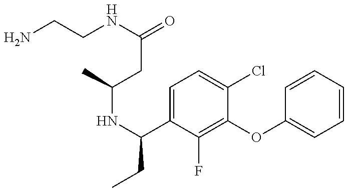 [SMILES: CC[C@@H](N[C@@H](C)CC(=O)NCCN)c1ccc(Cl)c(Oc2ccccc2)c1F] | 224 |
| 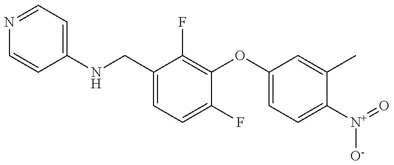 [SMILES: Cc1cc(Oc2c(F)ccc(CNc3ccncc3)c2F)ccc1[N+](=O)[O-]] | 225A |
| 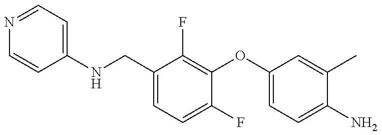 [SMILES: Cc1cc(Oc2c(F)ccc(CNc3ccncc3)c2F)ccc1N] | 225B |
| 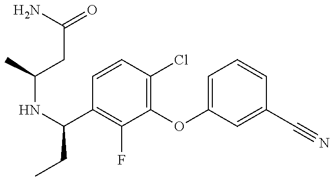 [SMILES: CC[C@@H](N[C@@H](C)CC(N)=O)c1ccc(Cl)c(Oc2cccc(C#N)c2)c1F] | 226 |
| 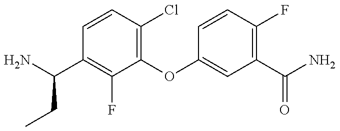 [SMILES: CC[C@@H](N)c1ccc(Cl)c(Oc2ccc(F)c(C(N)=O)c2)c1F] | 227 |
| 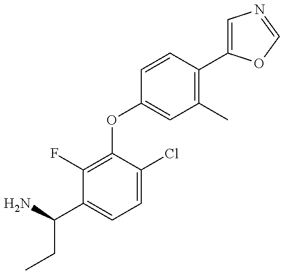 [SMILES: CC[C@@H](N)c1ccc(Cl)c(Oc2ccc(-c3cnco3)c(C)c2)c1F] | 228 |
| 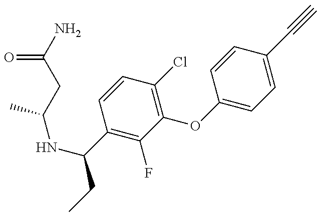 [SMILES: C#Cc1ccc(Oc2c(Cl)ccc([C@@H](CC)N[C@H](C)CC(N)=O)c2F)cc1] | 229 |
| 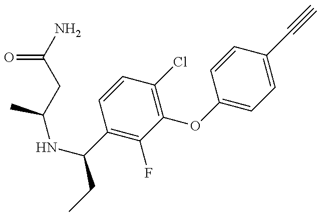 [SMILES: C#Cc1ccc(Oc2c(Cl)ccc([C@@H](CC)N[C@@H](C)CC(N)=O)c2F)cc1] | 230 |
| 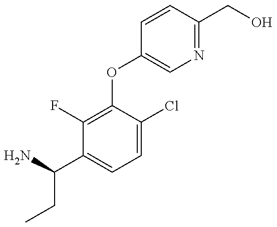 [SMILES: CC[C@@H](N)c1ccc(Cl)c(Oc2ccc(CO)nc2)c1F] | 231 |
| 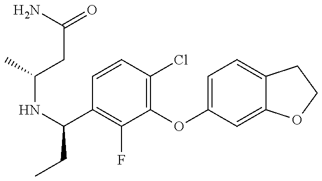 [SMILES: CC[C@@H](N[C@H](C)CC(N)=O)c1ccc(Cl)c(Oc2ccc3c(c2)OCC3)c1F] | 232 |
| 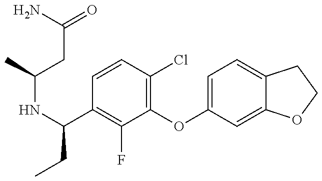 [SMILES: CC[C@@H](N[C@@H](C)CC(N)=O)c1ccc(Cl)c(Oc2ccc3c(c2)OCC3)c1F] | 233 |
| 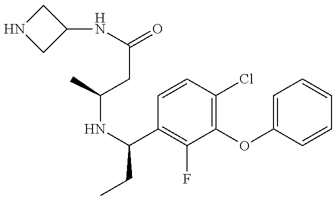 [SMILES: CC[C@@H](N[C@@H](C)CC(=O)NC1CNC1)c1ccc(Cl)c(Oc2ccccc2)c1F] | 234 |
| 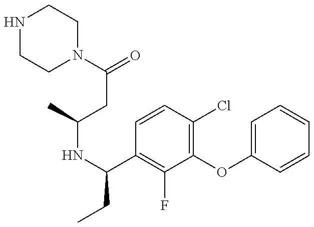 [SMILES: CC[C@@H](N[C@@H](C)CC(=O)N1CCNCC1)c1ccc(Cl)c(Oc2ccccc2)c1F] | 235 |
| 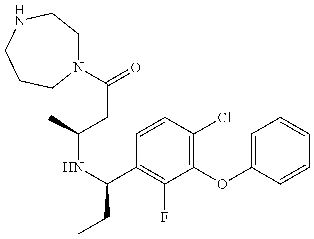 [SMILES: CC[C@@H](N[C@@H](C)CC(=O)N1CCCNCC1)c1ccc(Cl)c(Oc2ccccc2)c1F] | 236 |
| 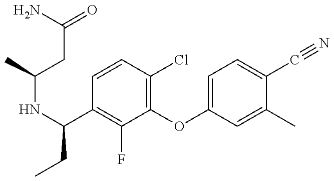 [SMILES: CC[C@@H](N[C@@H](C)CC(N)=O)c1ccc(Cl)c(Oc2ccc(C#N)c(C)c2)c1F] | 237 |
| 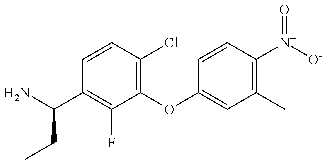 [SMILES: CC[C@@H](N)c1ccc(Cl)c(Oc2ccc([N+](=O)[O-])c(C)c2)c1F] | 238A |
| 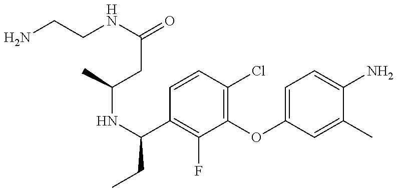 [SMILES: CC[C@@H](N[C@@H](C)CC(=O)NCCN)c1ccc(Cl)c(Oc2ccc(N)c(C)c2)c1F] | 238B |
| 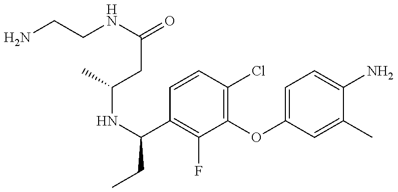 [SMILES: CC[C@@H](N[C@H](C)CC(=O)NCCN)c1ccc(Cl)c(Oc2ccc(N)c(C)c2)c1F] | 239 |
| 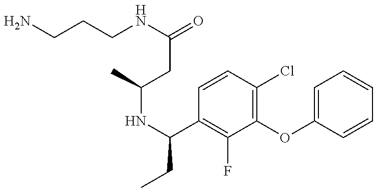 [SMILES: CC[C@@H](N[C@@H](C)CC(=O)NCCCN)c1ccc(Cl)c(Oc2ccccc2)c1F] | 240 |
| 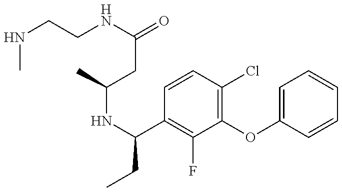 [SMILES: CC[C@@H](N[C@@H](C)CC(=O)NCCNC)c1ccc(Cl)c(Oc2ccccc2)c1F] | 241 |
| 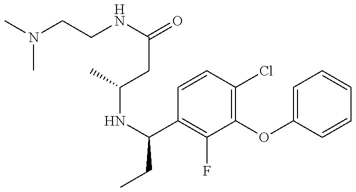 [SMILES: CC[C@@H](N[C@H](C)CC(=O)NCCN(C)C)c1ccc(Cl)c(Oc2ccccc2)c1F] | 242 |
| 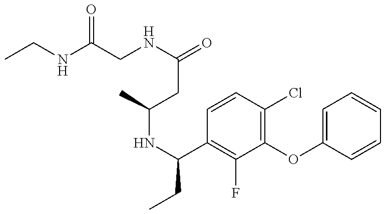 [SMILES: CCNC(=O)CNC(=O)C[C@H](C)N[C@H](CC)c1ccc(Cl)c(Oc2ccccc2)c1F] | 243 |
| 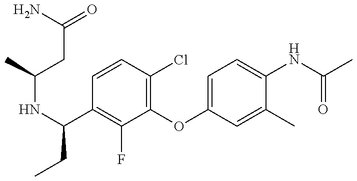 [SMILES: CC[C@@H](N[C@@H](C)CC(N)=O)c1ccc(Cl)c(Oc2ccc(NC(C)=O)c(C)c2)c1F] | 244 |
| 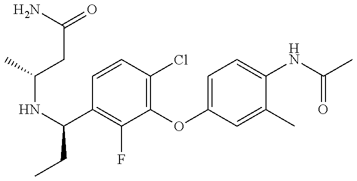 [SMILES: CC[C@@H](N[C@H](C)CC(N)=O)c1ccc(Cl)c(Oc2ccc(NC(C)=O)c(C)c2)c1F] | 245 |
| 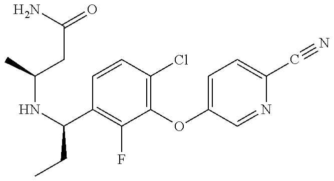 [SMILES: CC[C@@H](N[C@@H](C)CC(N)=O)c1ccc(Cl)c(Oc2ccc(C#N)nc2)c1F] | 246 |
| 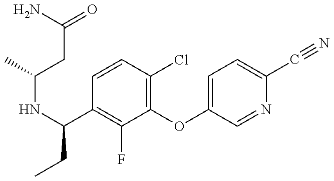 [SMILES: CC[C@@H](N[C@H](C)CC(N)=O)c1ccc(Cl)c(Oc2ccc(C#N)nc2)c1F] | 247 |
| 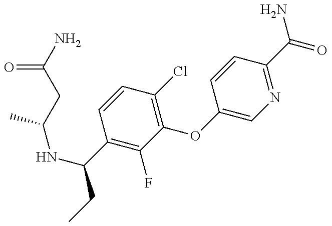 [SMILES: CC[C@@H](N[C@H](C)CC(N)=O)c1ccc(Cl)c(Oc2ccc(C(N)=O)nc2)c1F] | 248 |
| 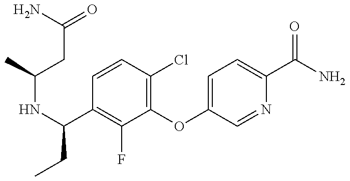 [SMILES: CC[C@@H](N[C@@H](C)CC(N)=O)c1ccc(Cl)c(Oc2ccc(C(N)=O)nc2)c1F] | 249 |
| 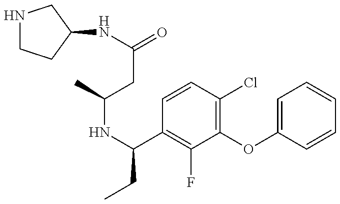 [SMILES: CC[C@@H](N[C@@H](C)CC(=O)N[C@H]1CCNC1)c1ccc(Cl)c(Oc2ccccc2)c1F] | 250 |
| 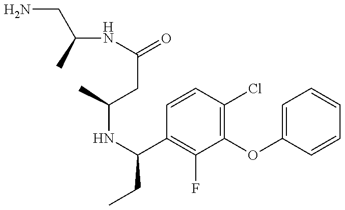 [SMILES: CC[C@@H](N[C@@H](C)CC(=O)N[C@@H](C)CN)c1ccc(Cl)c(Oc2ccccc2)c1F] | 251 |
| 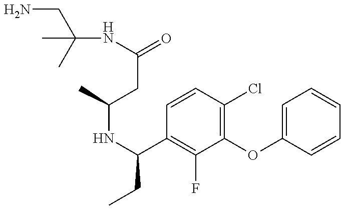 [SMILES: CC[C@@H](N[C@@H](C)CC(=O)NC(C)(C)CN)c1ccc(Cl)c(Oc2ccccc2)c1F] | 252 |
| 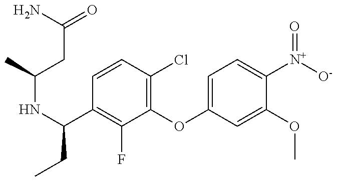 [SMILES: CC[C@@H](N[C@@H](C)CC(N)=O)c1ccc(Cl)c(Oc2ccc([N+](=O)[O-])c(OC)c2)c1F] | 253 |
| 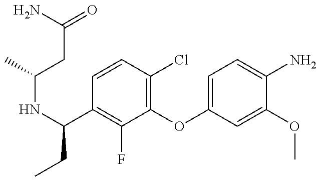 [SMILES: CC[C@@H](N[C@H](C)CC(N)=O)c1ccc(Cl)c(Oc2ccc(N)c(OC)c2)c1F] | 254 |
| 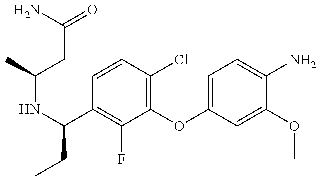 [SMILES: CC[C@@H](N[C@@H](C)CC(N)=O)c1ccc(Cl)c(Oc2ccc(N)c(OC)c2)c1F] | 255 |
| 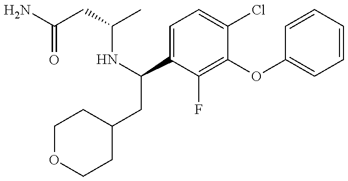 [SMILES: C[C@@H](CC(N)=O)N[C@H](CC1CCOCC1)c1ccc(Cl)c(Oc2ccccc2)c1F] | 256 |
| 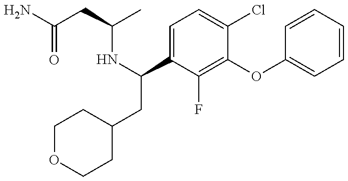 [SMILES: C[C@H](CC(N)=O)N[C@H](CC1CCOCC1)c1ccc(Cl)c(Oc2ccccc2)c1F] | 257 |
| 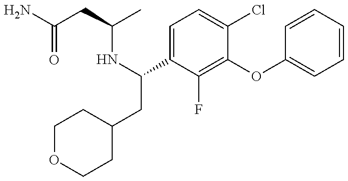 [SMILES: C[C@H](CC(N)=O)N[C@@H](CC1CCOCC1)c1ccc(Cl)c(Oc2ccccc2)c1F] | 258 |
| 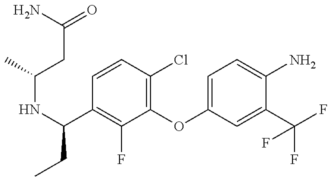 [SMILES: CC[C@@H](N[C@H](C)CC(N)=O)c1ccc(Cl)c(Oc2ccc(N)c(C(F)(F)F)c2)c1F] | 259 |
| 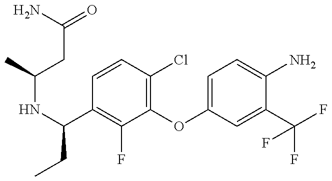 [SMILES: CC[C@@H](N[C@@H](C)CC(N)=O)c1ccc(Cl)c(Oc2ccc(N)c(C(F)(F)F)c2)c1F] | 260 |
| 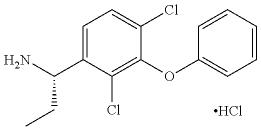 [SMILES: CC[C@H](N)c1ccc(Cl)c(Oc2ccccc2)c1Cl ; Cl] | 261 |
| 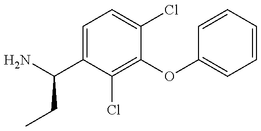 [SMILES: CC[C@@H](N)c1ccc(Cl)c(Oc2ccccc2)c1Cl] | 262 |
| 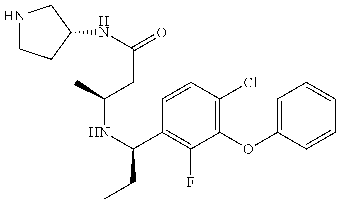 [SMILES: CC[C@@H](N[C@@H](C)CC(=O)N[C@@H]1CCNC1)c1ccc(Cl)c(Oc2ccccc2)c1F] | 263 |
| 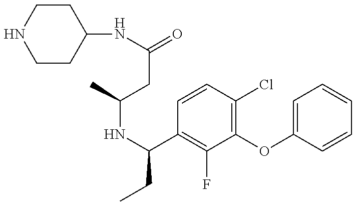 [SMILES: CC[C@@H](N[C@@H](C)CC(=O)NC1CCNCC1)c1ccc(Cl)c(Oc2ccccc2)c1F] | 264 |
| 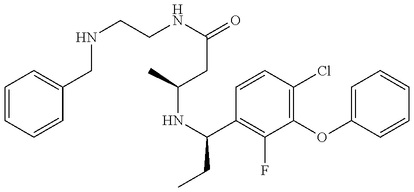 [SMILES: CC[C@@H](N[C@@H](C)CC(=O)NCCNCc1ccccc1)c1ccc(Cl)c(Oc2ccccc2)c1F] | 265 |
| 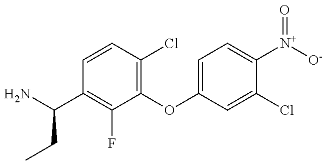 [SMILES: CC[C@@H](N)c1ccc(Cl)c(Oc2ccc([N+](=O)[O-])c(Cl)c2)c1F] | 266A |
| 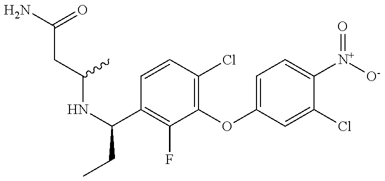 [SMILES: CC[C@@H](NC(C)CC(N)=O)c1ccc(Cl)c(Oc2ccc([N+](=O)[O-])c(Cl)c2)c1F] | 266B |
| 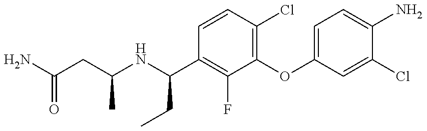 [SMILES: CC[C@@H](N[C@@H](C)CC(N)=O)c1ccc(Cl)c(Oc2ccc(N)c(Cl)c2)c1F] | 266C |
| 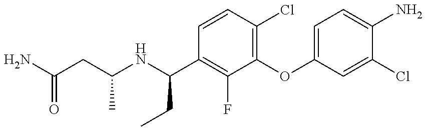 [SMILES: CC[C@@H](N[C@H](C)CC(N)=O)c1ccc(Cl)c(Oc2ccc(N)c(Cl)c2)c1F] | 267 |
| 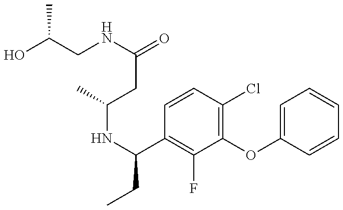 [SMILES: CC[C@@H](N[C@H](C)CC(=O)NC[C@@H](C)O)c1ccc(Cl)c(Oc2ccccc2)c1F] | 268 |
| 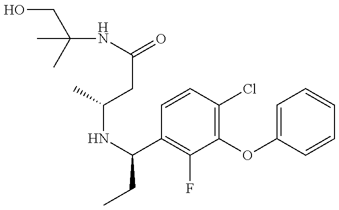 [SMILES: CC[C@@H](N[C@H](C)CC(=O)NC(C)(C)CO)c1ccc(Cl)c(Oc2ccccc2)c1F] | 269 |
| 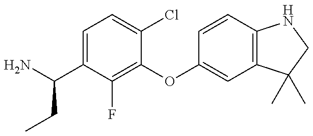 [SMILES: CC[C@@H](N)c1ccc(Cl)c(Oc2ccc3c(c2)C(C)(C)CN3)c1F] | 270 |
| 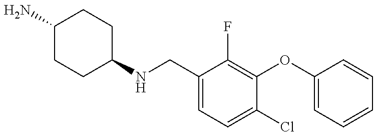 [SMILES: N[C@H]1CC[C@H](NCc2ccc(Cl)c(Oc3ccccc3)c2F)CC1] | 271 |
| 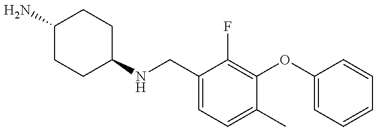 [SMILES: Cc1ccc(CN[C@H]2CC[C@H](N)CC2)c(F)c1Oc1ccccc1] | 272 |
| 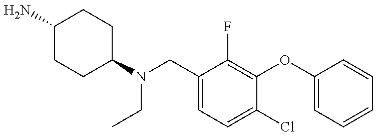 [SMILES: CCN(Cc1ccc(Cl)c(Oc2ccccc2)c1F)[C@H]1CC[C@H](N)CC1] | 273B |
| 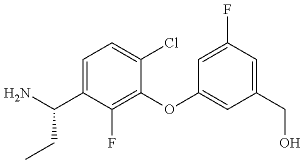 [SMILES: CC[C@H](N)c1ccc(Cl)c(Oc2cc(F)cc(CO)c2)c1F] | 274 |
| 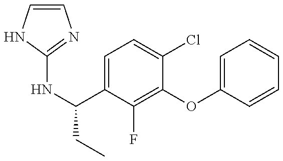 [SMILES: CC[C@H](Nc1ncc[nH]1)c1ccc(Cl)c(Oc2ccccc2)c1F] | 275 |
| 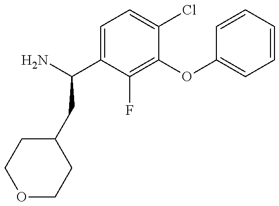 [SMILES: N[C@H](CC1CCOCC1)c1ccc(Cl)c(Oc2ccccc2)c1F] | 276A |
| 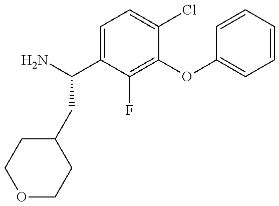 [SMILES: N[C@@H](CC1CCOCC1)c1ccc(Cl)c(Oc2ccccc2)c1F] | 276B |
| 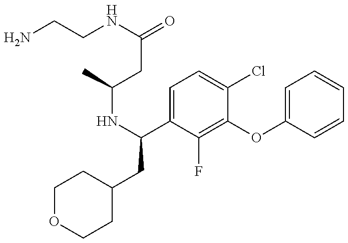 [SMILES: C[C@@H](CC(=O)NCCN)N[C@H](CC1CCOCC1)c1ccc(Cl)c(Oc2ccccc2)c1F] | 277 |
| 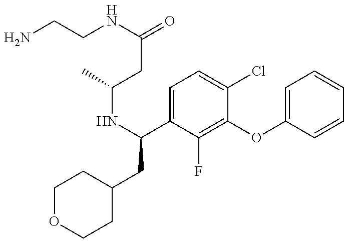 [SMILES: C[C@H](CC(=O)NCCN)N[C@H](CC1CCOCC1)c1ccc(Cl)c(Oc2ccccc2)c1F] | 278 |
| 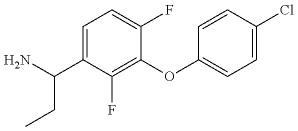 [SMILES: CCC(N)c1ccc(F)c(Oc2ccc(Cl)cc2)c1F] | 279 |
| 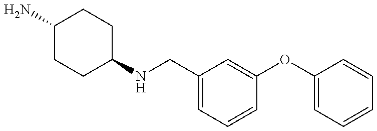 [SMILES: N[C@H]1CC[C@H](NCc2cccc(Oc3ccccc3)c2)CC1] | 280 |
| 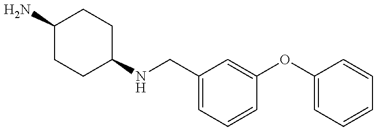 [SMILES: N[C@H]1CC[C@@H](NCc2cccc(Oc3ccccc3)c2)CC1] | 281 |
| 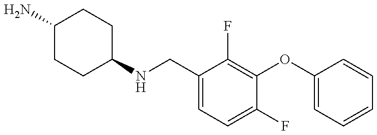 [SMILES: N[C@H]1CC[C@H](NCc2ccc(F)c(Oc3ccccc3)c2F)CC1] | 282 |
| 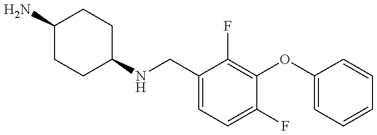 [SMILES: N[C@H]1CC[C@@H](NCc2ccc(F)c(Oc3ccccc3)c2F)CC1] | 283 |
| 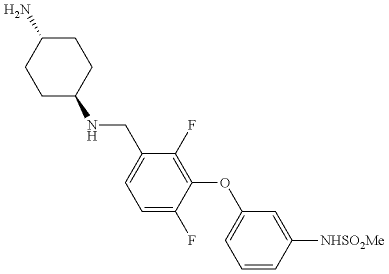 | 284 |
| 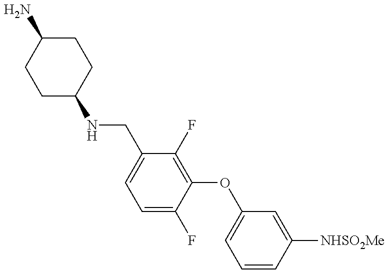 | 285 |
| 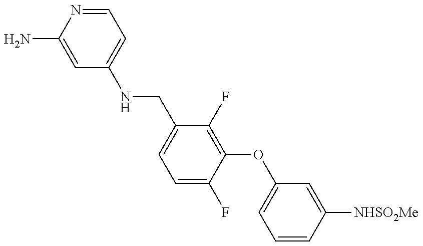 | 286 |
| 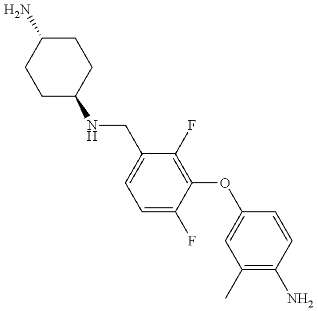 [SMILES: Cc1cc(Oc2c(F)ccc(CN[C@H]3CC[C@H](N)CC3)c2F)ccc1N] | 287 |
| 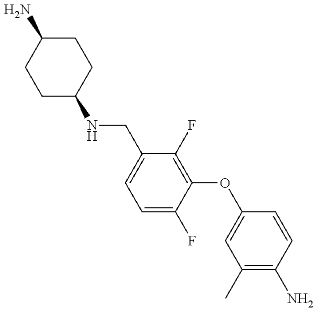 [SMILES: Cc1cc(Oc2c(F)ccc(CN[C@H]3CC[C@@H](N)CC3)c2F)ccc1N] | 288 |
| 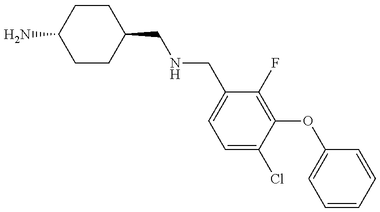 [SMILES: N[C@H]1CC[C@H](CNCc2ccc(Cl)c(Oc3ccccc3)c2F)CC1] | 289 |
| 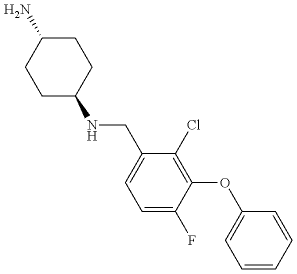 [SMILES: N[C@H]1CC[C@H](NCc2ccc(F)c(Oc3ccccc3)c2Cl)CC1] | 290 |
| 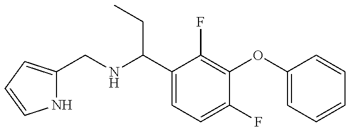 [SMILES: CCC(NCc1ccc[nH]1)c1ccc(F)c(Oc2ccccc2)c1F] | 291 |
| 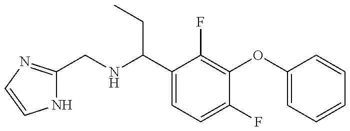 [SMILES: CCC(NCc1ncc[nH]1)c1ccc(F)c(Oc2ccccc2)c1F] | 292 |
| 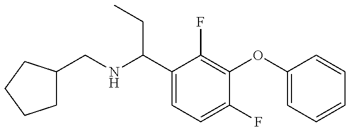 [SMILES: CCC(NCC1CCCC1)c1ccc(F)c(Oc2ccccc2)c1F] | 293 |
| 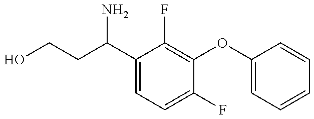 [SMILES: NC(CCO)c1ccc(F)c(Oc2ccccc2)c1F] | 294 |
| 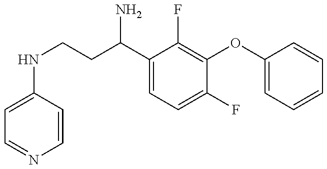 [SMILES: NC(CCNc1ccncc1)c1ccc(F)c(Oc2ccccc2)c1F] | 295 |
| 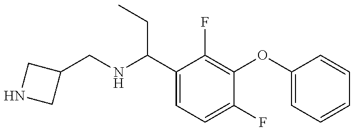 [SMILES: CCC(NCC1CNC1)c1ccc(F)c(Oc2ccccc2)c1F] | 296 |
| 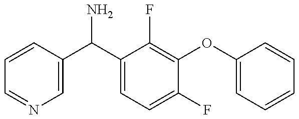 [SMILES: NC(c1cccnc1)c1ccc(F)c(Oc2ccccc2)c1F] | 297 |
| 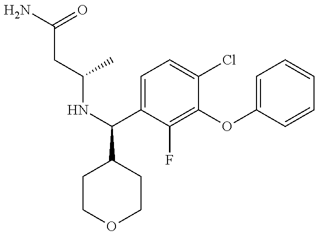 [SMILES: C[C@@H](CC(N)=O)N[C@@H](c1ccc(Cl)c(Oc2ccccc2)c1F)C1CCOCC1] | 299 |
| 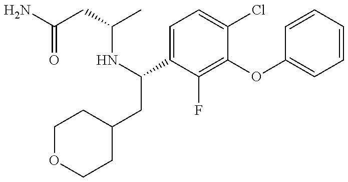 [SMILES: C[C@@H](CC(N)=O)N[C@@H](CC1CCOCC1)c1ccc(Cl)c(Oc2ccccc2)c1F] | 300 |
| 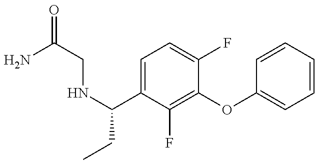 [SMILES: CC[C@H](NCC(N)=O)c1ccc(F)c(Oc2ccccc2)c1F] | 301 |
| 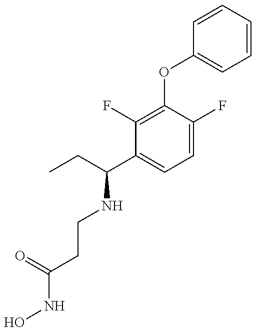 [SMILES: CC[C@H](NCCC(=O)NO)c1ccc(F)c(Oc2ccccc2)c1F] | 302 |
| 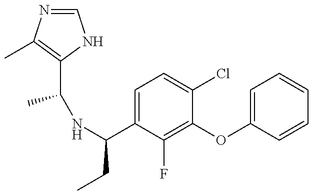 [SMILES: CC[C@@H](N[C@H](C)c1[nH]cnc1C)c1ccc(Cl)c(Oc2ccccc2)c1F] | 303 |
| 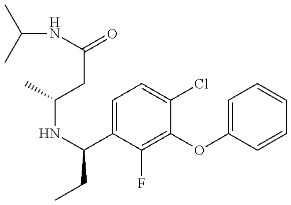 [SMILES: CC[C@@H](N[C@H](C)CC(=O)NC(C)C)c1ccc(Cl)c(Oc2ccccc2)c1F] | 304 |
| 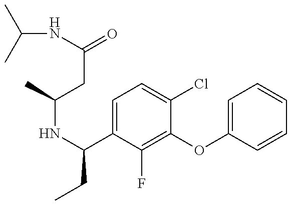 [SMILES: CC[C@@H](N[C@@H](C)CC(=O)NC(C)C)c1ccc(Cl)c(Oc2ccccc2)c1F] | 305 |
| 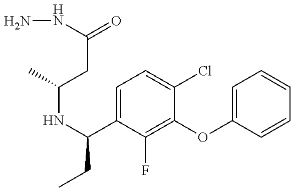 [SMILES: CC[C@@H](N[C@H](C)CC(=O)NN)c1ccc(Cl)c(Oc2ccccc2)c1F] | 306 |
| 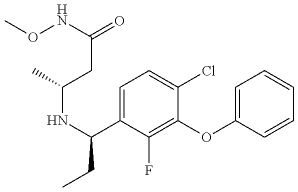 [SMILES: CC[C@@H](N[C@H](C)CC(=O)NOC)c1ccc(Cl)c(Oc2ccccc2)c1F] | 307 |
| 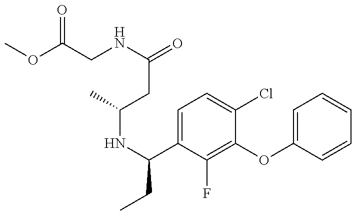 [SMILES: CC[C@@H](N[C@H](C)CC(=O)NCC(=O)OC)c1ccc(Cl)c(Oc2ccccc2)c1F] | 308 |
| 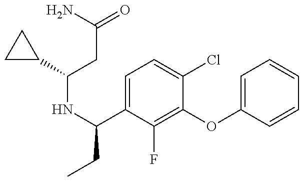 [SMILES: CC[C@@H](N[C@@H](CC(N)=O)C1CC1)c1ccc(Cl)c(Oc2ccccc2)c1F] | 309 |
| 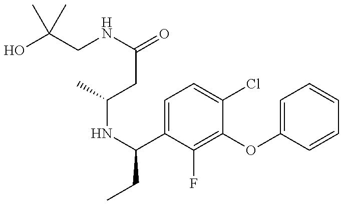 [SMILES: CC[C@@H](N[C@H](C)CC(=O)NCC(C)(C)O)c1ccc(Cl)c(Oc2ccccc2)c1F] | 310 |
| 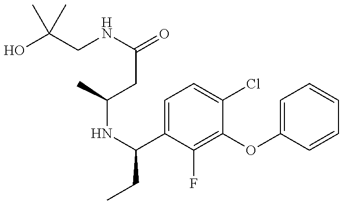 [SMILES: CC[C@@H](N[C@@H](C)CC(=O)NCC(C)(C)O)c1ccc(Cl)c(Oc2ccccc2)c1F] | 311 |
| 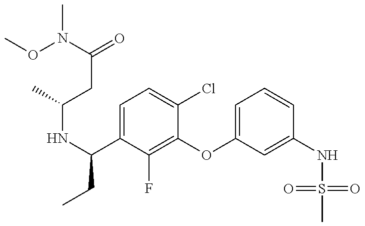 [SMILES: CC[C@@H](N[C@H](C)CC(=O)N(C)OC)c1ccc(Cl)c(Oc2cccc(NS(C)(=O)=O)c2)c1F] | 312 |
| 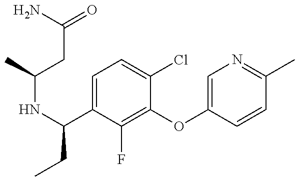 [SMILES: CC[C@@H](N[C@@H](C)CC(N)=O)c1ccc(Cl)c(Oc2ccc(C)nc2)c1F] | 313 |
| 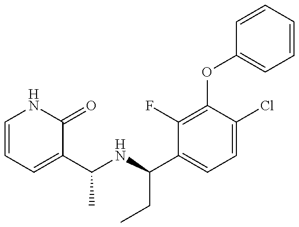 [SMILES: CC[C@@H](N[C@H](C)c1ccc[nH]c1=O)c1ccc(Cl)c(Oc2ccccc2)c1F] | 314 |
| 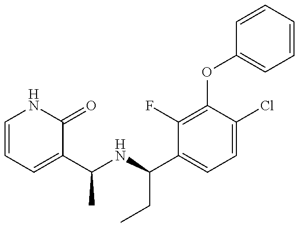 [SMILES: CC[C@@H](N[C@@H](C)c1ccc[nH]c1=O)c1ccc(Cl)c(Oc2ccccc2)c1F] | 315 |
| 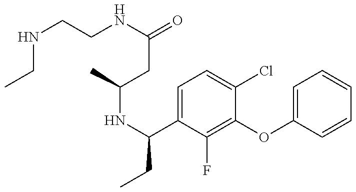 [SMILES: CCNCCNC(=O)C[C@H](C)N[C@H](CC)c1ccc(Cl)c(Oc2ccccc2)c1F] | 316 |
| 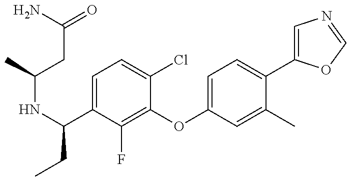 [SMILES: CC[C@@H](N[C@@H](C)CC(N)=O)c1ccc(Cl)c(Oc2ccc(-c3cnco3)c(C)c2)c1F] | 317 |
| 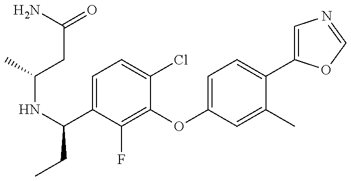 [SMILES: CC[C@@H](N[C@H](C)CC(N)=O)c1ccc(Cl)c(Oc2ccc(-c3cnco3)c(C)c2)c1F] | 318 |
| 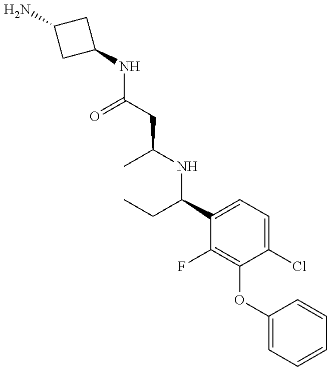 [SMILES: CC[C@@H](N[C@@H](C)CC(=O)N[C@H]1C[C@H](N)C1)c1ccc(Cl)c(Oc2ccccc2)c1F] | 319 |
| 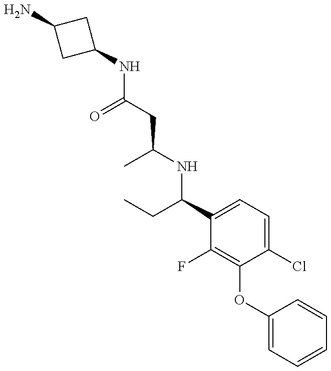 [SMILES: CC[C@@H](N[C@@H](C)CC(=O)N[C@H]1C[C@@H](N)C1)c1ccc(Cl)c(Oc2ccccc2)c1F] | 320 |
| 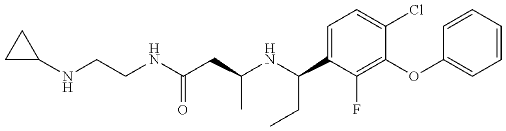 [SMILES: CC[C@@H](N[C@@H](C)CC(=O)NCCNC1CC1)c1ccc(Cl)c(Oc2ccccc2)c1F] | 321 |
| 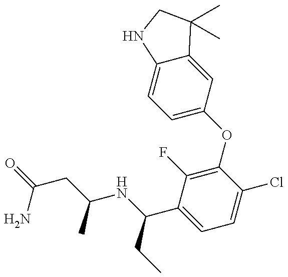 [SMILES: CC[C@@H](N[C@@H](C)CC(N)=O)c1ccc(Cl)c(Oc2ccc3c(c2)C(C)(C)CN3)c1F] | 322 |
| 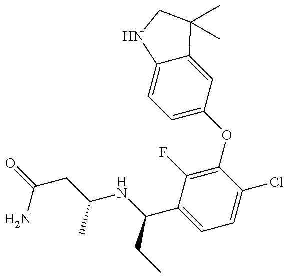 [SMILES: CC[C@@H](N[C@H](C)CC(N)=O)c1ccc(Cl)c(Oc2ccc3c(c2)C(C)(C)CN3)c1F] | 323 |
| 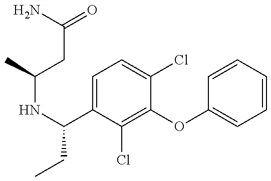 [SMILES: CC[C@H](N[C@@H](C)CC(N)=O)c1ccc(Cl)c(Oc2ccccc2)c1Cl] | 324 |
| 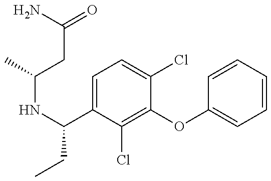 [SMILES: CC[C@H](N[C@H](C)CC(N)=O)c1ccc(Cl)c(Oc2ccccc2)c1Cl] | 325 |
| 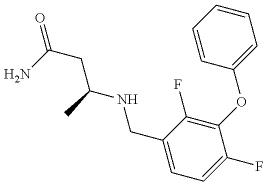 [SMILES: C[C@@H](CC(N)=O)NCc1ccc(F)c(Oc2ccccc2)c1F] | 26 |
| 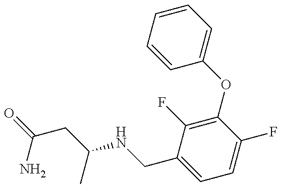 [SMILES: C[C@H](CC(N)=O)NCc1ccc(F)c(Oc2ccccc2)c1F] | 327 |
| 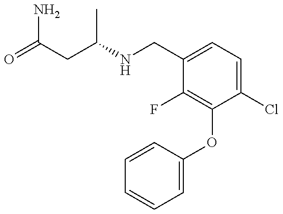 [SMILES: C[C@@H](CC(N)=O)NCc1ccc(Cl)c(Oc2ccccc2)c1F] | 328 |
| 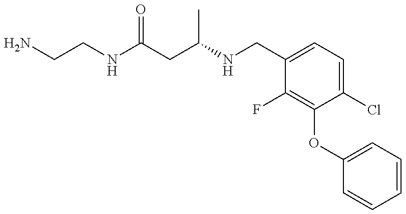 [SMILES: C[C@@H](CC(=O)NCCN)NCc1ccc(Cl)c(Oc2ccccc2)c1F] | 329 |
| 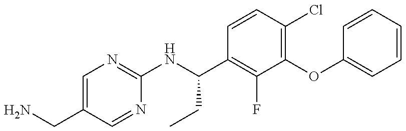 [SMILES: CC[C@H](Nc1ncc(CN)cn1)c1ccc(Cl)c(Oc2ccccc2)c1F] | 330 |
| 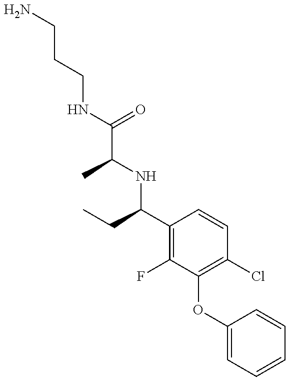 [SMILES: CC[C@@H](N[C@@H](C)C(=O)NCCCN)c1ccc(Cl)c(Oc2ccccc2)c1F] | 331 |
| 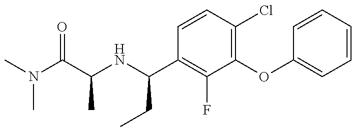 [SMILES: CC[C@@H](N[C@@H](C)C(=O)N(C)C)c1ccc(Cl)c(Oc2ccccc2)c1F] | 332 |
| 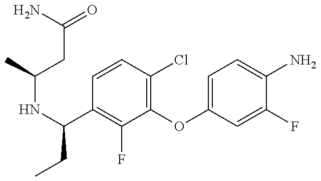 [SMILES: CC[C@@H](N[C@@H](C)CC(N)=O)c1ccc(Cl)c(Oc2ccc(N)c(F)c2)c1F] | 333 |
| 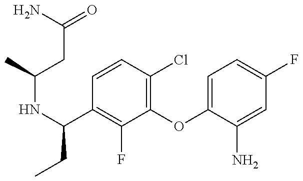 [SMILES: CC[C@@H](N[C@@H](C)CC(N)=O)c1ccc(Cl)c(Oc2ccc(F)cc2N)c1F] | 334 |
| 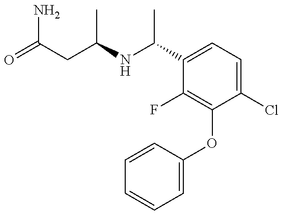 [SMILES: C[C@H](CC(N)=O)N[C@H](C)c1ccc(Cl)c(Oc2ccccc2)c1F] | 335 |
| 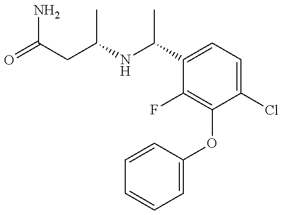 [SMILES: C[C@@H](CC(N)=O)N[C@H](C)c1ccc(Cl)c(Oc2ccccc2)c1F] | 336 |
| 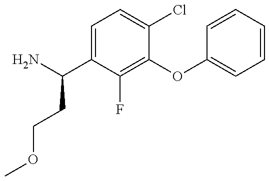 [SMILES: COCC[C@@H](N)c1ccc(Cl)c(Oc2ccccc2)c1F] | 337 |
| 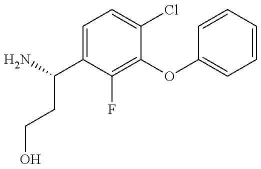 [SMILES: N[C@@H](CCO)c1ccc(Cl)c(Oc2ccccc2)c1F] | 338 |
| 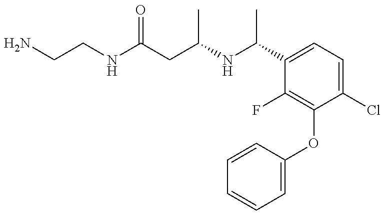 [SMILES: C[C@@H](CC(=O)NCCN)N[C@H](C)c1ccc(Cl)c(Oc2ccccc2)c1F] | 339 |
| 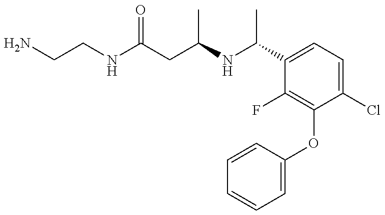 [SMILES: C[C@H](CC(=O)NCCN)N[C@H](C)c1ccc(Cl)c(Oc2ccccc2)c1F] | 340 |
| 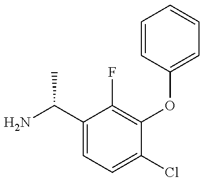 [SMILES: C[C@@H](N)c1ccc(Cl)c(Oc2ccccc2)c1F] | 341 |
| 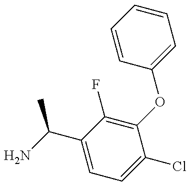 [SMILES: C[C@H](N)c1ccc(Cl)c(Oc2ccccc2)c1F] | 342 |
| 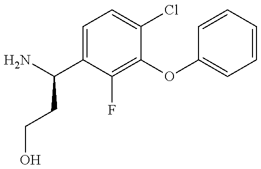 [SMILES: N[C@H](CCO)c1ccc(Cl)c(Oc2ccccc2)c1F] | 343 |
| 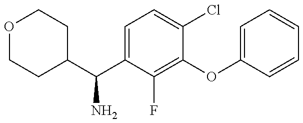 [SMILES: N[C@H](c1ccc(Cl)c(Oc2ccccc2)c1F)C1CCOCC1] | 344 |
| 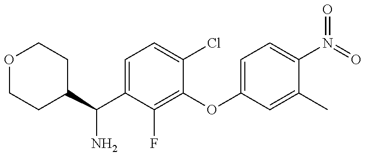 [SMILES: Cc1cc(Oc2c(Cl)ccc([C@@H](N)C3CCOCC3)c2F)ccc1[N+](=O)[O-]] | 345 |
| 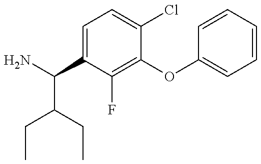 [SMILES: CCC(CC)[C@@H](N)c1ccc(Cl)c(Oc2ccccc2)c1F] | 346 |
| 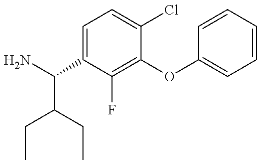 [SMILES: CCC(CC)[C@H](N)c1ccc(Cl)c(Oc2ccccc2)c1F] | 347 |
| 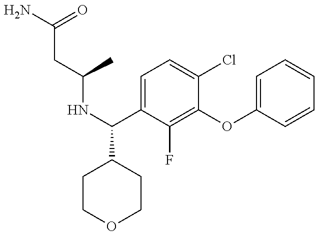 [SMILES: C[C@H](CC(N)=O)N[C@H](c1ccc(Cl)c(Oc2ccccc2)c1F)C1CCOCC1] | 348 |
| 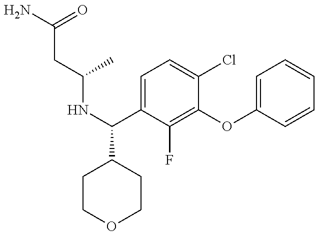 [SMILES: C[C@@H](CC(N)=O)N[C@H](c1ccc(Cl)c(Oc2ccccc2)c1F)C1CCOCC1] | 349 |
| 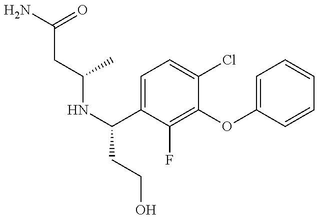 [SMILES: C[C@@H](CC(N)=O)N[C@@H](CCO)c1ccc(Cl)c(Oc2ccccc2)c1F] | 350 |
| 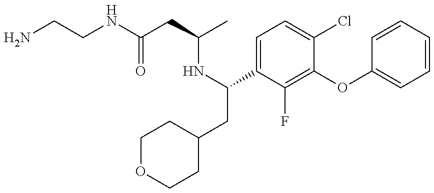 [SMILES: C[C@H](CC(=O)NCCN)N[C@@H](CC1CCOCC1)c1ccc(Cl)c(Oc2ccccc2)c1F] | 351 |
| 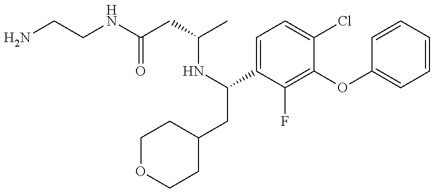 [SMILES: C[C@@H](CC(=O)NCCN)N[C@@H](CC1CCOCC1)c1ccc(Cl)c(Oc2ccccc2)c1F] | 352 |
| 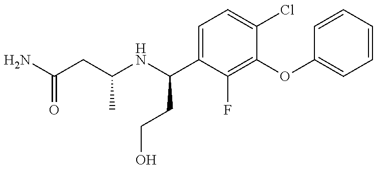 [SMILES: C[C@H](CC(N)=O)N[C@H](CCO)c1ccc(Cl)c(Oc2ccccc2)c1F] | 353 |
| 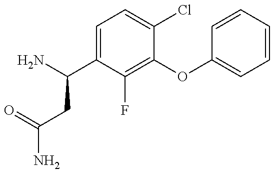 [SMILES: NC(=O)C[C@@H](N)c1ccc(Cl)c(Oc2ccccc2)c1F] | 354 |
| 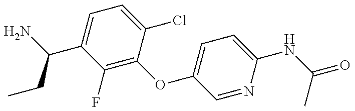 [SMILES: CC[C@@H](N)c1ccc(Cl)c(Oc2ccc(NC(C)=O)nc2)c1F] | 355 |
| 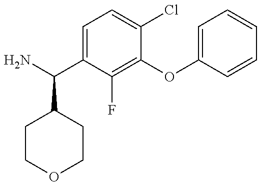 [SMILES: N[C@@H](c1ccc(Cl)c(Oc2ccccc2)c1F)C1CCOCC1] | 356 |
| 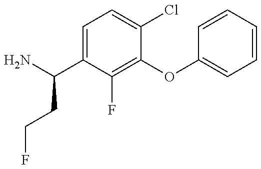 [SMILES: N[C@H](CCF)c1ccc(Cl)c(Oc2ccccc2)c1F] | 357 |
| 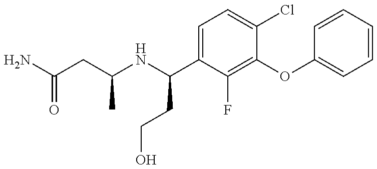 [SMILES: C[C@@H](CC(N)=O)N[C@H](CCO)c1ccc(Cl)c(Oc2ccccc2)c1F] | 358 |
| 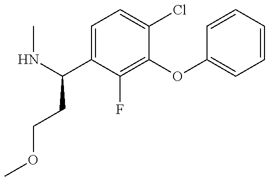 [SMILES: CN[C@H](CCOC)c1ccc(Cl)c(Oc2ccccc2)c1F] | 359 |
| 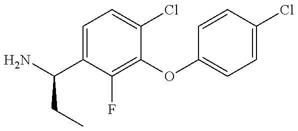 [SMILES: CC[C@@H](N)c1ccc(Cl)c(Oc2ccc(Cl)cc2)c1F] | 360 |
| 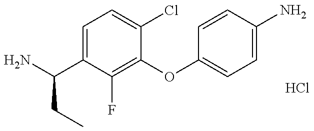 [SMILES: CC[C@@H](N)c1ccc(Cl)c(Oc2ccc(N)cc2)c1F ; Cl] | 361 |
| 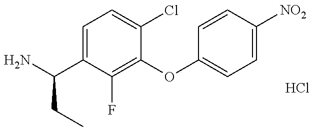 [SMILES: CC[C@@H](N)c1ccc(Cl)c(Oc2ccc([N+](=O)[O-])cc2)c1F ; Cl] | 362 |
| 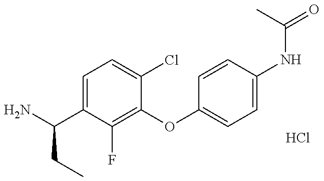 [SMILES: CC[C@@H](N)c1ccc(Cl)c(Oc2ccc(NC(C)=O)cc2)c1F ; Cl] | 363 |
| 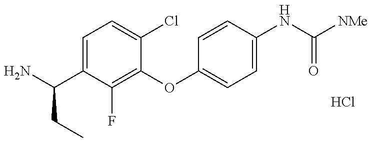 | 364 |
| 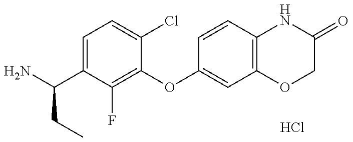 [SMILES: CC[C@@H](N)c1ccc(Cl)c(Oc2ccc3c(c2)OCC(=O)N3)c1F ; Cl] | 365 |
| 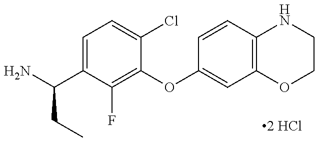 [SMILES: CC[C@@H](N)c1ccc(Cl)c(Oc2ccc3c(c2)OCCN3)c1F] | 366 |
| 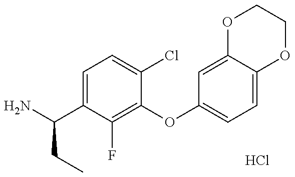 [SMILES: CC[C@@H](N)c1ccc(Cl)c(Oc2ccc3c(c2)OCCO3)c1F ; Cl] | 367 |
| 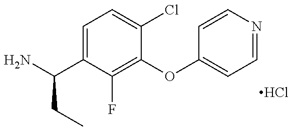 [SMILES: CC[C@@H](N)c1ccc(Cl)c(Oc2ccncc2)c1F ; Cl] | 368 |
| 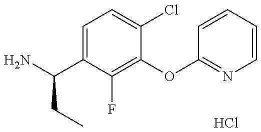 [SMILES: CC[C@@H](N)c1ccc(Cl)c(Oc2ccccn2)c1F ; Cl] | 369 |
| 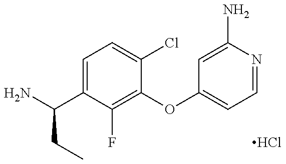 [SMILES: CC[C@@H](N)c1ccc(Cl)c(Oc2ccnc(N)c2)c1F ; Cl] | 370 |
| 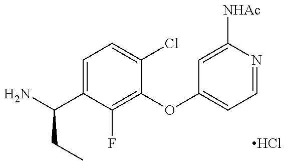 [SMILES: CC[C@@H](N)c1ccc(Cl)c(Oc2ccnc(NC(C)=O)c2)c1F ; Cl] | 371 |
| 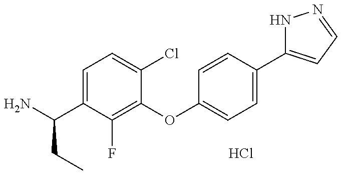 [SMILES: CC[C@@H](N)c1ccc(Cl)c(Oc2ccc(-c3ccn[nH]3)cc2)c1F ; Cl] | 372 |
| 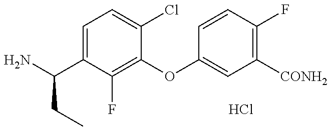 [SMILES: CC[C@@H](N)c1ccc(Cl)c(Oc2ccc(F)c(C(N)=O)c2)c1F ; Cl] | 373 |
| 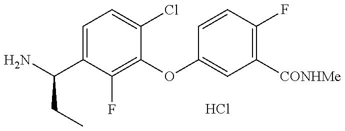 | 374 |
| 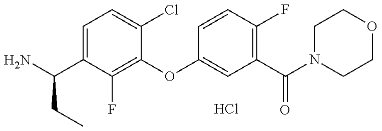 [SMILES: CC[C@@H](N)c1ccc(Cl)c(Oc2ccc(F)c(C(=O)N3CCOCC3)c2)c1F ; Cl] | 375 |
| 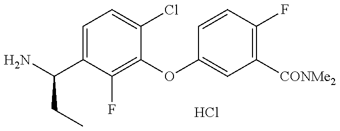 | 376 |
| 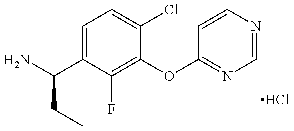 [SMILES: CC[C@@H](N)c1ccc(Cl)c(Oc2ccncn2)c1F ; Cl] | 377 |
| 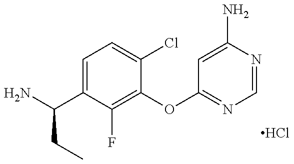 [SMILES: CC[C@@H](N)c1ccc(Cl)c(Oc2cc(N)ncn2)c1F ; Cl] | 378 |
| 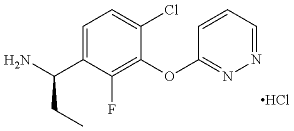 [SMILES: CC[C@@H](N)c1ccc(Cl)c(Oc2cccnn2)c1F ; Cl] | 379 |
| 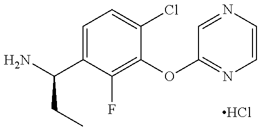 [SMILES: CC[C@@H](N)c1ccc(Cl)c(Oc2cnccn2)c1F ; Cl] | 380 |
| 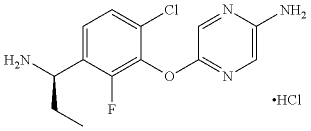 [SMILES: CC[C@@H](N)c1ccc(Cl)c(Oc2cnc(N)cn2)c1F ; Cl] | 381 |
| 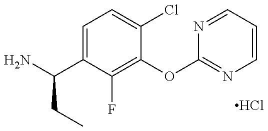 [SMILES: CC[C@@H](N)c1ccc(Cl)c(Oc2ncccn2)c1F ; Cl] | 382 |
| 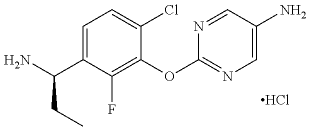 [SMILES: CC[C@@H](N)c1ccc(Cl)c(Oc2ncc(N)cn2)c1F ; Cl] | 383 |
| 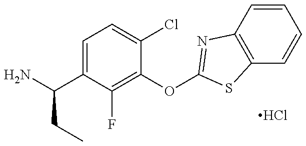 [SMILES: CC[C@@H](N)c1ccc(Cl)c(Oc2nc3ccccc3s2)c1F ; Cl] | 384 |
| 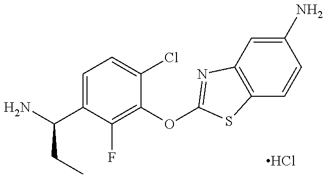 [SMILES: CC[C@@H](N)c1ccc(Cl)c(Oc2nc3cc(N)ccc3s2)c1F ; Cl] | 385 |
| 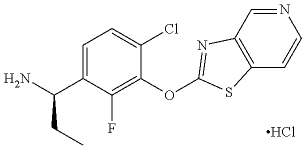 [SMILES: CC[C@@H](N)c1ccc(Cl)c(Oc2nc3cnccc3s2)c1F ; Cl] | 386 |
| 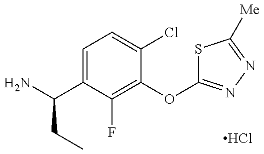 [SMILES: CC[C@@H](N)c1ccc(Cl)c(Oc2nnc(C)s2)c1F ; Cl] | 387 |
| 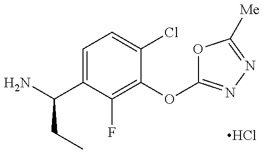 [SMILES: CC[C@@H](N)c1ccc(Cl)c(Oc2nnc(C)o2)c1F ; Cl] | 388 |
| 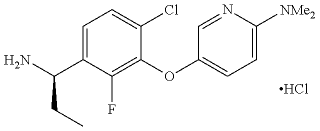 [SMILES: CC[C@@H](N)c1ccc(Cl)c(Oc2ccc(N(C)C)nc2)c1F ; Cl] | 389 |
| 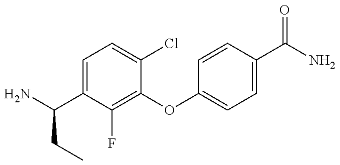 [SMILES: CC[C@@H](N)c1ccc(Cl)c(Oc2ccc(C(N)=O)cc2)c1F] | 390 |
| 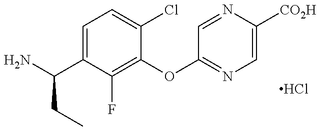 [SMILES: CC[C@@H](N)c1ccc(Cl)c(Oc2cnc(C(=O)O)cn2)c1F ; Cl] | 391 |
| 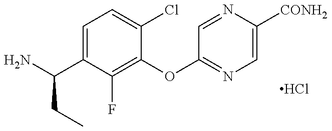 [SMILES: CC[C@@H](N)c1ccc(Cl)c(Oc2cnc(C(N)=O)cn2)c1F ; Cl] | 392 |
| 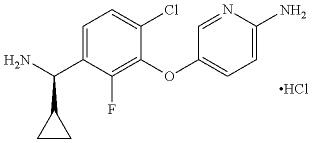 [SMILES: Nc1ccc(Oc2c(Cl)ccc([C@H](N)C3CC3)c2F)cn1 ; Cl] | 397 |
| 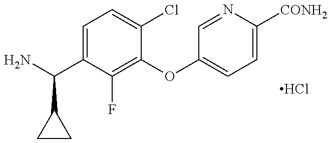 [SMILES: NC(=O)c1ccc(Oc2c(Cl)ccc([C@H](N)C3CC3)c2F)cn1 ; Cl] | 398 |
| 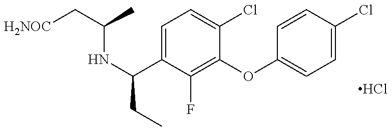 [SMILES: CC[C@@H](N[C@H](C)CC(N)=O)c1ccc(Cl)c(Oc2ccc(Cl)cc2)c1F ; Cl] | 399 |
| 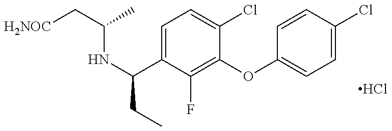 [SMILES: CC[C@@H](N[C@@H](C)CC(N)=O)c1ccc(Cl)c(Oc2ccc(Cl)cc2)c1F ; Cl] | 400 |
| 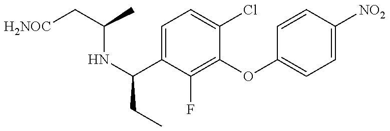 [SMILES: CC[C@@H](N[C@H](C)CC(N)=O)c1ccc(Cl)c(Oc2ccc([N+](=O)[O-])cc2)c1F] | 401 |
| 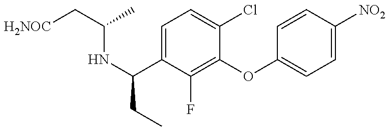 [SMILES: CC[C@@H](N[C@@H](C)CC(N)=O)c1ccc(Cl)c(Oc2ccc([N+](=O)[O-])cc2)c1F] | 402 |
| 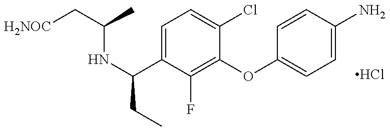 [SMILES: CC[C@@H](N[C@H](C)CC(N)=O)c1ccc(Cl)c(Oc2ccc(N)cc2)c1F ; Cl] | 403 |
| 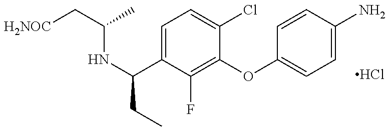 [SMILES: CC[C@@H](N[C@@H](C)CC(N)=O)c1ccc(Cl)c(Oc2ccc(N)cc2)c1F ; Cl] | 404 |
| 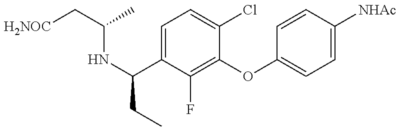 [SMILES: CC[C@@H](N[C@@H](C)CC(N)=O)c1ccc(Cl)c(Oc2ccc(NC(C)=O)cc2)c1F] | 405 |
| 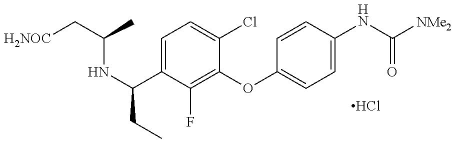 [SMILES: CC[C@@H](N[C@H](C)CC(N)=O)c1ccc(Cl)c(Oc2ccc(NC(=O)N(C)C)cc2)c1F ; Cl] | 406 |
| 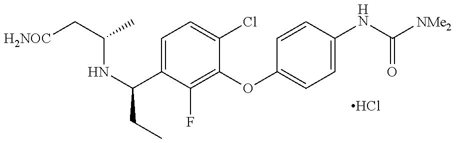 [SMILES: CC[C@@H](N[C@@H](C)CC(N)=O)c1ccc(Cl)c(Oc2ccc(NC(=O)N(C)C)cc2)c1F ; Cl] | 407 |
| 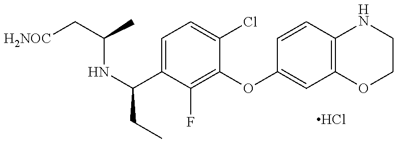 [SMILES: CC[C@@H](N[C@H](C)CC(N)=O)c1ccc(Cl)c(Oc2ccc3c(c2)OCCN3)c1F ; Cl] | 408 |
| 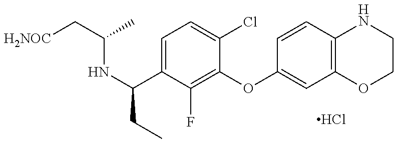 [SMILES: CC[C@@H](N[C@@H](C)CC(N)=O)c1ccc(Cl)c(Oc2ccc3c(c2)OCCN3)c1F ; Cl] | 409 |
| 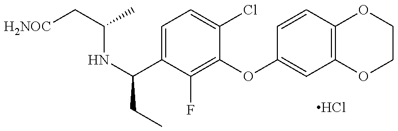 [SMILES: CC[C@@H](N[C@@H](C)CC(N)=O)c1ccc(Cl)c(Oc2ccc3c(c2)OCCO3)c1F ; Cl] | 410 |
| 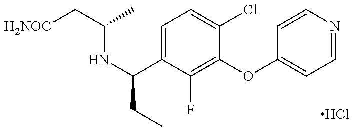 [SMILES: CC[C@@H](N[C@@H](C)CC(N)=O)c1ccc(Cl)c(Oc2ccncc2)c1F ; Cl] | 411 |
| 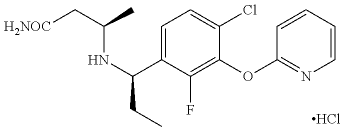 [SMILES: CC[C@@H](N[C@H](C)CC(N)=O)c1ccc(Cl)c(Oc2ccccn2)c1F ; Cl] | 412 |
| 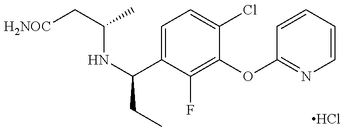 [SMILES: CC[C@@H](N[C@@H](C)CC(N)=O)c1ccc(Cl)c(Oc2ccccn2)c1F ; Cl] | 413 |
| 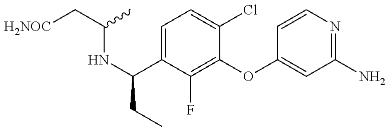 [SMILES: CC[C@@H](NC(C)CC(N)=O)c1ccc(Cl)c(Oc2ccnc(N)c2)c1F] | 414 |
| 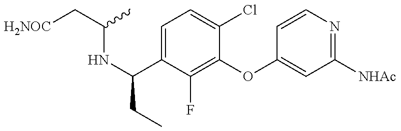 [SMILES: CC[C@@H](NC(C)CC(N)=O)c1ccc(Cl)c(Oc2ccnc(NC(C)=O)c2)c1F] | 415 |
| 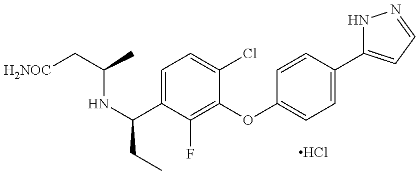 [SMILES: CC[C@@H](N[C@H](C)CC(N)=O)c1ccc(Cl)c(Oc2ccc(-c3ccn[nH]3)cc2)c1F ; Cl] | 416 |
| 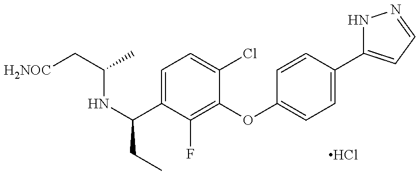 [SMILES: CC[C@@H](N[C@@H](C)CC(N)=O)c1ccc(Cl)c(Oc2ccc(-c3ccn[nH]3)cc2)c1F ; Cl] | 417 |
| 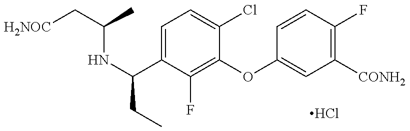 [SMILES: CC[C@@H](N[C@H](C)CC(N)=O)c1ccc(Cl)c(Oc2ccc(F)c(C(N)=O)c2)c1F ; Cl] | 418 |
| 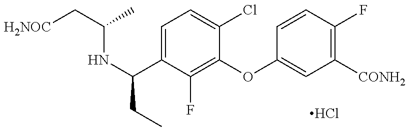 [SMILES: CC[C@@H](N[C@@H](C)CC(N)=O)c1ccc(Cl)c(Oc2ccc(F)c(C(N)=O)c2)c1F ; Cl] | 419 |
| 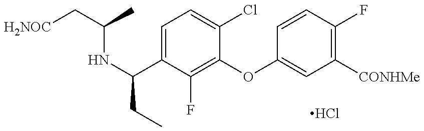 | 420 |
| 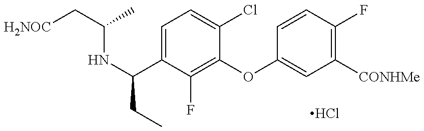 | 421 |
| 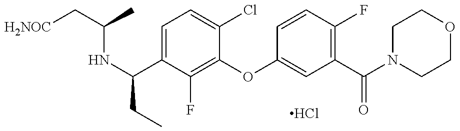 [SMILES: CC[C@@H](N[C@H](C)CC(N)=O)c1ccc(Cl)c(Oc2ccc(F)c(C(=O)N3CCOCC3)c2)c1F ; Cl] | 422 |
| 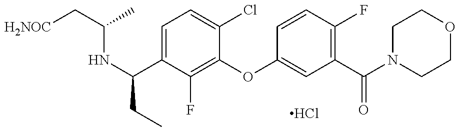 [SMILES: CC[C@@H](N[C@@H](C)CC(N)=O)c1ccc(Cl)c(Oc2ccc(F)c(C(=O)N3CCOCC3)c2)c1F ; Cl] | 423 |
| 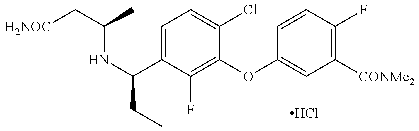 | 424 |
| 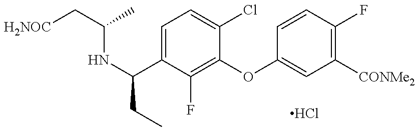 | 425 |
| 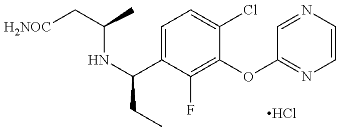 [SMILES: CC[C@@H](N[C@H](C)CC(N)=O)c1ccc(Cl)c(Oc2cnccn2)c1F ; Cl] | 426 |
| 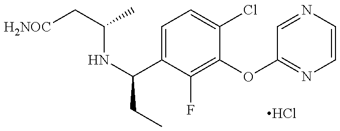 [SMILES: CC[C@@H](N[C@@H](C)CC(N)=O)c1ccc(Cl)c(Oc2cnccn2)c1F ; Cl] | 427 |
| 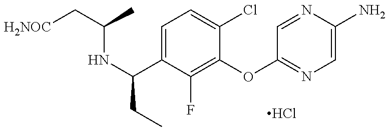 [SMILES: CC[C@@H](N[C@H](C)CC(N)=O)c1ccc(Cl)c(Oc2cnc(N)cn2)c1F ; Cl] | 428 |
| 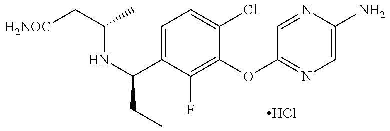 [SMILES: CC[C@@H](N[C@@H](C)CC(N)=O)c1ccc(Cl)c(Oc2cnc(N)cn2)c1F ; Cl] | 429 |
| 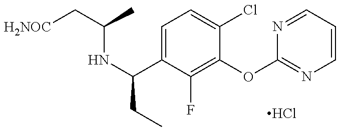 [SMILES: CC[C@@H](N[C@H](C)CC(N)=O)c1ccc(Cl)c(Oc2ncccn2)c1F ; Cl] | 430 |
| 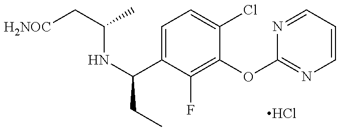 [SMILES: CC[C@@H](N[C@@H](C)CC(N)=O)c1ccc(Cl)c(Oc2ncccn2)c1F ; Cl] | 431 |
| 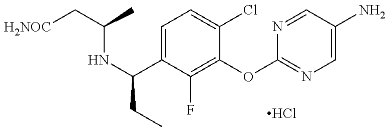 [SMILES: CC[C@@H](N[C@H](C)CC(N)=O)c1ccc(Cl)c(Oc2ncc(N)cn2)c1F ; Cl] | 432 |
| 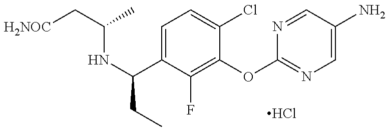 [SMILES: CC[C@@H](N[C@@H](C)CC(N)=O)c1ccc(Cl)c(Oc2ncc(N)cn2)c1F ; Cl] | 433 |
| 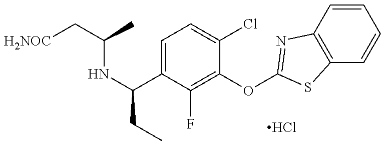 [SMILES: CC[C@@H](N[C@H](C)CC(N)=O)c1ccc(Cl)c(Oc2nc3ccccc3s2)c1F ; Cl] | 434 |
| 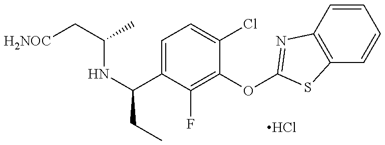 [SMILES: CC[C@@H](N[C@@H](C)CC(N)=O)c1ccc(Cl)c(Oc2nc3ccccc3s2)c1F ; Cl] | 435 |
| 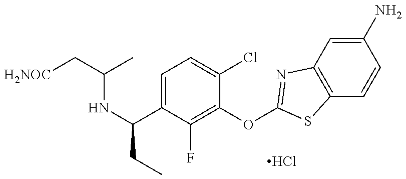 [SMILES: CC[C@@H](NC(C)CC(N)=O)c1ccc(Cl)c(Oc2nc3cc(N)ccc3s2)c1F ; Cl] | 436 |
| 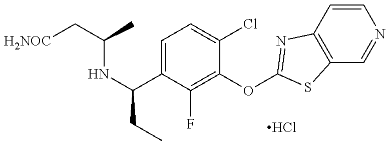 [SMILES: CC[C@@H](N[C@H](C)CC(N)=O)c1ccc(Cl)c(Oc2nc3ccncc3s2)c1F ; Cl] | 437 |
| 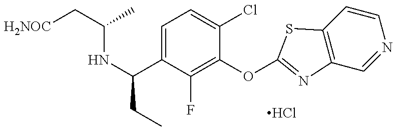 [SMILES: CC[C@@H](N[C@@H](C)CC(N)=O)c1ccc(Cl)c(Oc2nc3cnccc3s2)c1F ; Cl] | 438 |
| 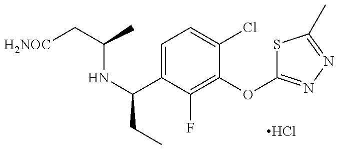 [SMILES: CC[C@@H](N[C@H](C)CC(N)=O)c1ccc(Cl)c(Oc2nnc(C)s2)c1F ; Cl] | 439 |
| 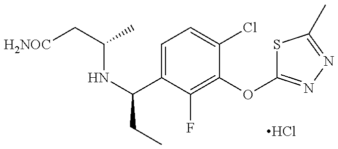 [SMILES: CC[C@@H](N[C@@H](C)CC(N)=O)c1ccc(Cl)c(Oc2nnc(C)s2)c1F ; Cl] | 440 |
| 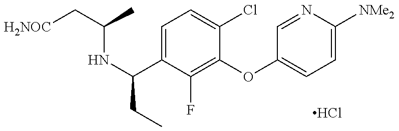 [SMILES: CC[C@@H](N[C@H](C)CC(N)=O)c1ccc(Cl)c(Oc2ccc(N(C)C)nc2)c1F ; Cl] | 441 |
| 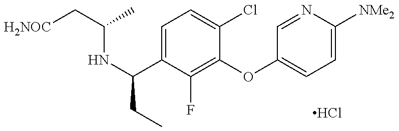 [SMILES: CC[C@@H](N[C@@H](C)CC(N)=O)c1ccc(Cl)c(Oc2ccc(N(C)C)nc2)c1F ; Cl] | 442 |
| 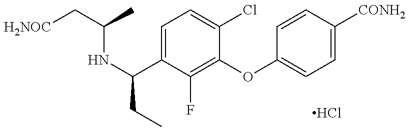 [SMILES: CC[C@@H](N[C@H](C)CC(N)=O)c1ccc(Cl)c(Oc2ccc(C(N)=O)cc2)c1F ; Cl] | 443 |
| 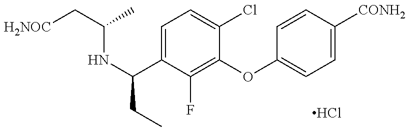 [SMILES: CC[C@@H](N[C@@H](C)CC(N)=O)c1ccc(Cl)c(Oc2ccc(C(N)=O)cc2)c1F ; Cl] | 444 |
| 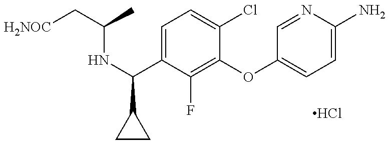 [SMILES: C[C@H](CC(N)=O)N[C@@H](c1ccc(Cl)c(Oc2ccc(N)nc2)c1F)C1CC1 ; Cl] | 453 |
| 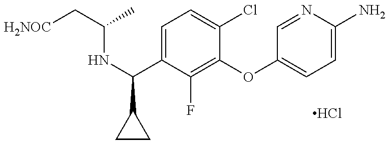 [SMILES: C[C@@H](CC(N)=O)N[C@@H](c1ccc(Cl)c(Oc2ccc(N)nc2)c1F)C1CC1 ; Cl] | 454 |
| 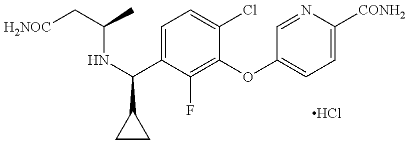 [SMILES: C[C@H](CC(N)=O)N[C@@H](c1ccc(Cl)c(Oc2ccc(C(N)=O)nc2)c1F)C1CC1 ; Cl] | 455 |
| 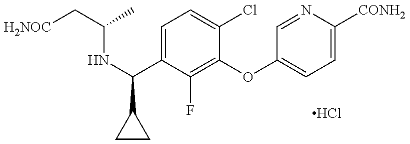 [SMILES: C[C@@H](CC(N)=O)N[C@@H](c1ccc(Cl)c(Oc2ccc(C(N)=O)nc2)c1F)C1CC1 ; Cl] | 456 |
| 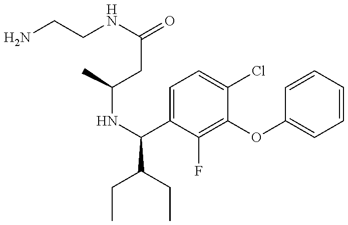 [SMILES: CCC(CC)[C@@H](N[C@@H](C)CC(=O)NCCN)c1ccc(Cl)c(Oc2ccccc2)c1F] | 457 |
| 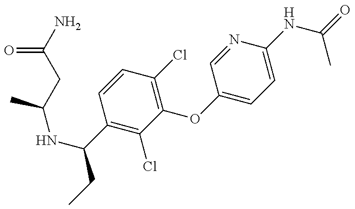 [SMILES: CC[C@@H](N[C@@H](C)CC(N)=O)c1ccc(Cl)c(Oc2ccc(NC(C)=O)nc2)c1Cl] | 458 |
| 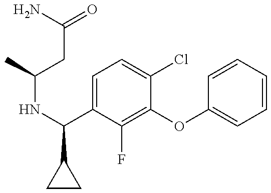 [SMILES: C[C@@H](CC(N)=O)N[C@@H](c1ccc(Cl)c(Oc2ccccc2)c1F)C1CC1] | 459 |
| 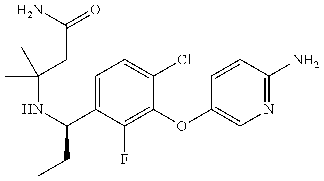 [SMILES: CC[C@@H](NC(C)(C)CC(N)=O)c1ccc(Cl)c(Oc2ccc(N)nc2)c1F] | 460 |
| 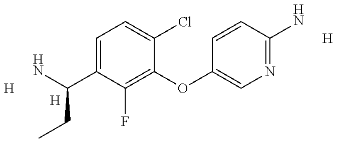 | 461 |
| 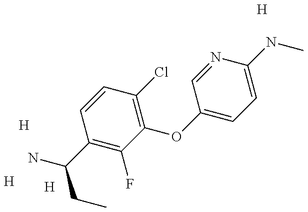 | 462 |
| 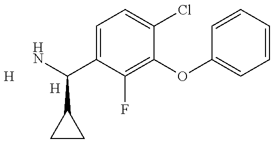 | 463 |
| 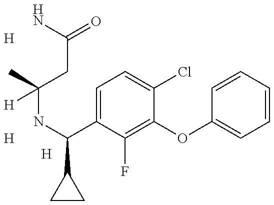 | 464 |
| 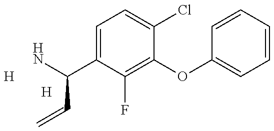 | 465 |
| 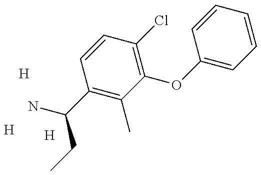 | 466 |
| 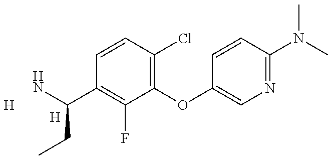 | 467 |
| 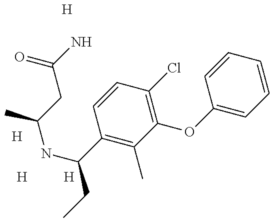 | 468 |
| 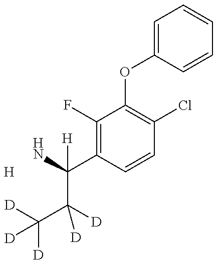 | 469 |
| 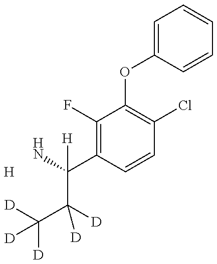 | 470 |
| 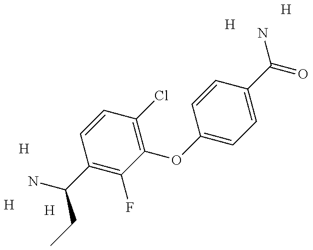 | 471 |
| 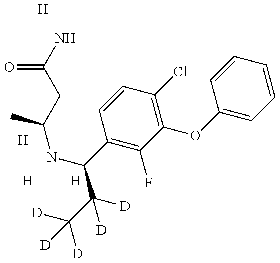 | 472 |
| 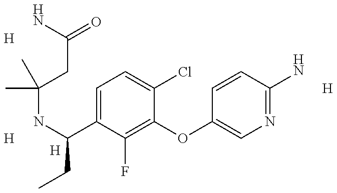 | 473 |

TABLE B
Characterising data and synthetic methods

| Example | NMR & MS Data | Synthetic Method |
|---|---|---|
| 1 | 1H NMR (400 MHz, DMSO-d6): 8.70 (3H, br s), 7.68-7.56 (1H, m), 7.47 (1H, t), 7.43-7.32 (2H, m), | Example 1 |
| | 7.18-7.06 (1H, m), 6.97 (2H, d), 4.20 (1H, dd), 2.28-2.16 (1H, m), 1.07 (3H, d), 0.78 (3H, d). [M + H − NH3]+ 261 | |
| 2 | 1H NMR (400 MHz, DMSO-d6): 8.62 (3H, s), 7.73-7.62 (1H, m), 7.52-7.43 (1H, m), 7.43-7.32 (2H, | As Example 1 using iso-butyl lithium in |
| | m), 7.25-7.07 (1H, m), 6.97 (2H, d), 4.50 (1H, t), 1.83 (2H, t), 1.45-1.32 (1H, m), 0.87 (6H, d). [M − NH2] + | step 3. |
| | 275 | |
| 3 | 1H NMR (400 MHz, DMSO-d6): 8.63 (3H, br s), 7.70-7.58 (1H, m), 7.53-7.43 (1H, m), 7.39 (2H, t), | Example 3, Step 1. |
| | 7.14 (1H, t), 6.97 (2H, d), 4.39 (1H, s), 2.10-1.97 (1H, m), 1.93-1.80 (1H, m), 0.82 (3H, t). [M + H − NH3]+ | |
| | 247 | |
| 4 | 1H NMR (400 MHz, DMSO-d6): 8.61 (3H, s), 7.69-7.57 (1H, m), 7.51-7.42 (1H, m), 7.41-7.33 (2H, | As Example 1 using methyl lithium in step |
| | m), 7.19-7.08 (1H, m), 6.98 (2H, d), 4.68-4.57 (1H, m), 1.56 (3H, d). [M − NH2] +233 | 3. |
| 5 | 1H NMR (400 MHz, DMSO-d6): 9.69 (1H, s), 9.58 (1H, s), 8.08 (3H, s), 7.97-7.88 (1H, m), | As Example 5/6. |
| | 7.52 (1H, t), 7.38 (2H, t), 7.13 (1H, t), 6.99 (2H, d), 4.54 (1H, d), 3.25 (1H, s), 2.98 (1H, s), | |
| | 2.36-2.25 (1H, m), 2.09-1.96 (1H, m), 1.96-1.59 (8H, m), 0.73 (3H, t). [M + H]+ 361 | |
| 6 | 1H NMR (400 MHz, DMSO-d6): 9.69 (1H, br s), 9.58 (1H, br s), 8.08 (3H, s), 7.98-7.88 (1H, m), | As Example 5/6. |
| | 7.52 (1H, t), 7.43-7.33 (2H, m), 7.13 (1H, t), 6.99 (2H, d), 4.54 (1H, br s), 3.25 (1H, br s), 2.98 (1H, | |
| | br s), 2.36-2.25 (1H, m), 2.09-1.96 (1H, m), 1.96-1.81 (5H, m), 1.81-1.59 (3H, m), 0.73 (3H, t). | |
| | [M + H]+ 361 | |
| 7 | ¹H NMR (400 MHz, DMSO-d₆, 80degC): 8.40 (2H, s), 8.30 (1H, d), 7.61 (1H, d), 7.52-7.43 (1H, m), | Example 7 |
| | 7.43-7.32 (3H, m), 7.10 (1H, t), 6.88 (2H, d), 6.75 (1H, d), 5.27 (1H, t), 3.94 (2H, s), 2.00-1.88 (1H, | |
| | m), 1.88-1.76 (1H, m), 0.93 (3H, t). [M + H]+ 387 | |
| 8 | ¹H NMR (400 MHz, DMSO-d₆): 8.39 (2H, s), 8.30 (2H, s), 8.17 (1H, d), 7.49-7.33 (4H, m), | Example 8 |
| | 7.11 (1H, t), 6.86 (2H, d), CH signal obscured by water signal at 5.1 ppm, 3.83 (2H, d), 1.95-1.82 (1H, | |
| | m), 1.82-1.68 (1H, m), 0.90 (3H, t). [M + H]+ 387 | |
| 9 | ¹H NMR (400 MHz, DMSO-d₆): 9.92-9.77 (2H, m), 8.86 (1H, s), 8.08 (3H, s), 7.87-7.78 (1H, m), | Example 9 |
| | 7.69 (1H, d), 7.39 (2H, t), 7.13 (1H, t), 6.92 (2H, d), 4.37 (1H, s), 3.74 (1H, d), 3.45-3.21 (2H, m), | |
| | 2.97-2.81 (2H, m), 2.24 (1H, d), 2.05-1.94 (1H, m), 1.39 (3H, d), 0.71 (4H, t). [M + H]+ 394 | |
| 10 | 1H NMR (400 MHz, DMSO-d6): 8.61 (3H, s), 7.70-7.59 (1H, m), 7.47 (1H, t), 7.42-7.30 (2H, m), | As Example 1 using nButyl magnesium |
| | 7.14 (1H, t), 6.97 (2H, d), 4.45 (1H, t), 2.06-1.92 (1H, m), 1.92-1.79 (1H, m), 1.34-1.15 (3H, m), | chloride in step 3. |
| | 1.15-1.03 (1H, m), 0.84 (3H, t). [M − NH2] +275 | |
| 11 | 1H NMR (400 MHz, DMSO-d6): 8.65 (3H, s), 7.70-7.58 (1H, m), 7.53-7.43 (1H, m), 7.43-7.33 (2H, | As Example 1 using (R)-(+)-2-Methyl-2- |
| | m), 7.19-7.08 (1H, m), 6.97 (2H, d), 4.39 (1H, br s), 2.10-1.97 (1H, m), 1.93-1.80 (1H, m), | propane sulfonamide in step 2 and ethyl |
| | 0.82 (3H, t). | magnesium bromide in step 3. |
| | [M − NH2] +247 | |
| 12 | 1H NMR (400 MHz, DMSO-d6): 8.55 (3H, s), 7.68-7.57 (1H, m), 7.52-7.43 (1H, m), 7.43-7.33 (2H, | As Example 1 using nPropyl magnesium |
| | m), 7.14 (1H, t), 6.97 (2H, d), 4.47 (1H, dd), 2.01-1.90 (1H, m), 1.90-1.78 (1H, m), 1.31-1.11 (2H, | chloride in step 3. |
| | m), 0.87 (3H, t). [M − NH2] +251 | |
| 13 | 1H NMR (400 MHz, DMSO-d6): 9.22 (2H, s), 9.10 (3H, s), 7.65-7.55 (1H, m), 7.48 (1H, t), | Example 13 |
| | 7.43-7.34 (2H, m), 7.15 (1H, t), 6.99 (2H, d), 5.94 (1H, s), 5.19 (1H, s), 3.66 (2H, s), 3.14 (2H, s), | |
| | 2.20 (2H, s). | |
| 14 | 1H NMR (400 MHz, DMSO-d6): 8.82 (4H, br s), 7.73-7.63 (1H, m), 7.50 (1H, t), 7.39 (2H, t), | Example 14 |
| | 7.14 (1H, t), 6.99 (2H, d), 4.32 (1H, d), 3.43-3.34 (1H, m), 3.26-3.14 (1H, m), 2.83 (2H, t), | |
| | 2.31-2.18 (1H, m), 2.09 (1H, d), 1.62-1.43 (2H, m), 1.39-1.24 (1H, m). | |
| 15A | | Example 15 step 1. |
| 15B | 1H NMR (400 MHz, DMSO-d6): 9.12 (3H, br s), 8.52 (3H, br s), 7.81-7.71 (1H, m), 7.50 (1H, t), | Example 15 step 2. |
| | 7.38 (2H, dd), 7.14 (1H, t), 7.04 (2H, d), 4.84 (1H, t), 3.60-3.43 (2H, m). | |
| 16 | 1H NMR (400 MHz, DMSO-d6): 9.54 (1H, d), 9.34 (1H, s), 7.71 (1H, q), 7.51 (1H, t), 7.39 (2H, t), | Example 16 |
| | 7.14 (1H, t), 6.98 (2H, d), 4.48 (1H, s), 2.47 (3H, s), 2.00-1.83 (2H, m), 1.33-1.21 (1H, m), | |
| | 0.85 (6H, 2 × d). [M + H]+ 265 | |
| 17 | 1H NMR (400 MHz, DMSO-d6): 10.66 (1H, s), 9.99 (1H, s), 9.27 (1H, s), 9.16 (1H, s), 7.89 (1H, s), | As Example 5/6 using N-Boc-piperidin-3- |
| | 7.52 (1H, t), 7.38 (2H, dd), 7.14 (1H, t), 7.03 (2H, d), 4.59 (1H, s), 3.40 2H, m), 3.25-3.06 (2H, m), | one in step 1. |
| | 2.86-2.75 (1H, m), 2.29-2.10 (2H, m), 2.07-1.95 (1H, m), 1.95-1.85 (1H, m), 1.81-1.57 (2H, m), | |
| | 0.74 (3H, t). [M + H] +347 | |
| 18 | 1H NMR (400 MHz, DMSO-d6): 10.40 (1H, s), 9.24 (1H, s), 8.97 (1H, s), 7.98 (1H, s), 7.55 (1H, t), | As Example 5/6 using N-Boc-piperidin-3- |
| | 7.44-7.34 (2H, m), 7.14 (1H, t), 6.99 (2H, d), 4.64-4.54 (1H, m), 3.71-3.60 (1H, m), 3.28-3.13 (2H, | onein step1. |
| | m), 3.10-2.97 (1H, m), 2.84-2.73 (1H, m), 2.28-2.16 (1H, m), 2.12-2.00 (2H, m), 1.95-1.83 (1H, m), | |
| | 1.73 (1H, d), 1.61-1.48 (1H, m), 0.76 (3H, t). [M + H] +347 | |
| 19 | 1H NMR (400 MHz, DMSO-d6): 9.15 (3H, s), 7.79-7.68 (1H, m), 7.51 (1H, t), 7.39 (2H, t), | Example 19 |
| | 7.15 (1H, t), 7.03 (2H, d), 4.97 (1H, t), 3.67 (1H, dd), 3.50 (1H, dd), 3.45-3.38 (1H, m), 1.30 (6H, 2 × d). | |
| | [M + H − NH3]+ 290 | |
| 20 | 1H NMR (400 MHz, Me-d3-OD): 8.66 (2H, d), 8.19 (2H, d), 7.59-7.50 (1H, m), 7.40-7.25 (3H, m), | Example 20 |
| | 7.17-7.07 (1H, m), 6.96 (2H, d), 5.16 (1H, t), 4.85 (24H, s), 3.43 (2H, dd). | |
| 21 | 1H NMR (400 MHz, Me-d3-OD): 7.73 (1H, d), 7.49-7.39 (1H, m), 7.39-7.29 (3H, m), 7.12 (1H, t), | As Example 5/6 using Pyrazole-3- |
| | 6.96 (2H, d), 6.43 (1H, d), 4.54 (1H, dd), 4.32-4.13 (2H, m), 2.31-2.21 (1H, m), 2.11-2.00 (1H, m), | carbaldehyde in Step 1. |
| | 0.89 (3H, t). | |
| 22 | 1H NMR (400 MHz, DMSO-d6): 9.13 (1H, d), 9.05-8.95 (1H, m), 7.62-7.48 (2H, m), 7.44-7.34 (2H, | As Example 5/6 using Cyclopropane |
| | m), 7.19-7.09 (1H, m), 6.98 (2H, d), 4.46 (1H, d), 2.93-2.83 (1H, m), 2.73-2.63 (1H, m), | carboxaldehyde in Step 1. |
| | 2.20-2.10 (1H, m), 1.93-1.82 (1H, m), 1.05-0.95 (1H, m), 0.75 (3H, t), 0.58 (2H, d), 0.37-0.24 (2H, m). | |
| 23 | 1H NMR (400 MHz, Me-d3-OD): 7.68-7.58 (2H, m), 7.42-7.31 (3H, m), 7.13 (1H, t), 6.98 (2H, d), | As Example 5/6 using 1,2- |
| | 4.68-4.59 (1H, m), 4.49-4.40 (1H, m), 4.27 (1H, d), 3.78 (3H, s), 2.68 (3H, s), 2.36-2.26 (1H, m), | Dimethylimidazole-5-carbaldehyde in Step |
| | 2.17-2.07 (1H, m), 0.92 (3H, t). | 1. |
| 24 | 1H NMR (400 MHz, Me-d3-OD): 8.86 (1H, s), 7.62-7.55 (1H, m), 7.42-7.31 (3H, m), 7.13 (1H, t), | As Example 5/6 using 4-Methylimidazole-5-carbaldehyde |
| | 6.98 (2H, d), 4.62 (1H, dd), 4.44-4.35 (1H, m), 4.22 (1H, d), 3.53-3.47 (1H, m), 2.35 (4H, d), | in Step 1. |
| | 2.16-2.08 (1H, m), 0.92 (3H, t). | |
| 25 | 1H NMR (400 MHz, Me-d3-OD): 7.58 (1H, dd), 7.48 (1H, dd), 7.41-7.29 (2H, m), 7.11 (1H, t), | As Example 28 step 2 using 2- |
| | 6.90 (2H, d), 4.66 (1H, dd), 3.70-3.58 (1H, m), 3.49-3.43 (2H, m), 3.43-3.37 (2H, m), 2.64 (1H, dd), | methoxyethylamine |
| | 2.56 (1H, dd), 2.31-2.16 (1H, m), 2.13-1.98 (1H, m), 1.36 (3H, d), 0.93 (3H, t). [M + H]+ 423.2 | |
| 26 | 1H NMR (400 MHz, Me-d3-OD): 7.56 (1H, dd), 7.49 (1H, dd), 7.39-7.31 (2H, m), 7.10 (1H, t), | As Example 28, step 2 using 1-amino- |
| | 6.90 (2H, d), 4.60 (1H, dd), 3.61-3.48 (2H, m), 3.45-3.34 (1H, m), 2.59-2.47 (2H, m), 2.25-2.12 (1H, m), | cyclopropanemethanol hydrochloride |
| | 2.12-1.98 (1H, m), 1.32 (3H, d), 0.90 (3H, t), 0.83-0.66 (4H, m). [M + H]+ 435.2 | |
| 27 | 1H NMR (400 MHz, Me-d3-OD): 7.58-7.48 (1H, m), 7.41-7.30 (3H, m), 7.13 (1H, t), 6.96 (2H, d), | As Example 5/6 using 3-(2-Oxoethyl)- |
| | 4.51 (1H, d), 3.40-3.35 (2H, m), 3.18-3.06 (1H, m), 3.02-2.85 (2H, m), 2.70 (1H, t), 2.31-2.20 (1H, | piperidine-1-carboxylic acid tert-butyl |
| | m), 2.13-1.81 (4H, m), 1.80-1.62 (3H, m), 1.33-1.20 (1H, m), 0.90 (3H, t). | ester in Step 1. |
| 28 | ¹H NMR (400 MHz, Me-d₃-OD): 7.58 (1H, d), 7.48 (1H, t), 7.42-7.25 (2H, m), | Example 28 |
| | 7.17-7.05 (1H, m), 6.90 (2H, d), 4.71-4.58 (1H, m), 3.97 (2H, s), 3.75 (3H, s), 3.69-3.62 (1H, m), | |
| | 3.32 (38H, s), 2.76-2.56 (2H, m), 2.23 (1H, dd), 2.12-1.97 (1H, m), 1.39 (3H, d), 0.91 (3H, | |
| | t). MS: [M + H]⁺ 437. | |
| 29 | 1H NMR (400 MHz, Me-d3-OD): 7.56 (1H, q), 7.42-7.31 (3H, m), 7.13 (1H, t), 6.96 (2H, d), | As Example 5/6 using N-Boc 4- |
| | 4.57-4.49 (1H, m), 3.45 (2H, d), 3.10-2.97 (3H, m), 2.85 (1H, dd), 2.33-2.23 (1H, m), 2.17-2.00 (4H, m), | piperidinylcarbox-aldehyde in Step 1. |
| | 1.57-1.42 (2H, m), 0.90 (3H, t). | |
| 30 | [1H NMR (400 MHz, Me-d3-OD): 7.49-7.41 (1H, m), 7.41-7.30 (3H, m), 7.13 (1H, t), 6.95 (2H, d), | As Example 5/6 using Cyclopentanone in |
| | 4.48 (1H, dd), 3.52-3.40 (1H, m), 2.26-2.16 (1H, m), 2.14-1.98 (3H, m), 1.87-1.75 (2H, m), | Step 1. |
| | 1.74-1.54 (4H, m), 0.89 (3H, t). | |
| 31 | 1H NMR (400 MHz, Me-d3-OD): 7.51-7.38 (2H, m), 7.37-7.27 (3H, m), 7.22-7.04 (4H, m), | As Example 5/6 using Indole-3- |
| | 6.99 (1H, t), 6.91 (2H, d), 4.01 (1H, dd), 3.83 (2H, q), 1.97-1.83 (1H, m), 1.77-1.64 (1H, m), 0.82 (3H, t). | carbaldehyde in Step 1. |
| 32 | 1H NMR (400 MHz, Me-d3-OD): 7.52-7.44 (1H, m), 7.40-7.30 (3H, m), 7.12 (1H, t), 6.95 (2H, d), | As Example 5/6 using Hydroxyacetone in |
| | 4.66-4.58 (1H, m), 3.81-3.74 (1H, m), 3.60-3.50 (1H, m), 3.30-3.21 (1H, m), 2.25-2.18 (1H, m), | Step 1. |
| | 2.07-1.99 (1H, m), 1.37-1.26 (3H, m), 0.89 (3H, t). | |
| 33 | 1H NMR (400 MHz, Me-d3-OD): 7.69 (1H, d), 7.42-7.29 (4H, m), 7.13 (1H, t), 7.00-6.92 (2H, m), | As Example 5/6 using 5-Acetylpyrazole |
| | 6.32 (1H, d), 4.42-4.33 (2H, m), 2.12-2.05 (1H, m), 2.02-1.96 (1H, m), 1.68 (3H, dd), 0.81 (3H, t). | hydrochloride and 1 eq. triethylamine in |
| | | Step 1. |
| 34 | 1H NMR (400 MHz, Me-d3-OD): 7.33 (3H, t), 7.21-7.03 (2H, m), 6.90 (2H, d), 4.01 (1H, dd), | As Example 5/6 using Acetone in Step 1. |
| | 2.67-2.55 (1H, m), 1.98-1.84 (1H, m), 1.74-1.60 (1H, m), 1.04 (6H, dd), 0.82 (3H, t). | |
| 35 | 1H NMR (400 MHz, Me-d3-OD): 7.54-7.42 (1H, m), 7.42-7.26 (3H, m), 7.12 (1H, t), 6.95 (2H, d), | As Example 5/6 using Indole-5- |
| | 4.66 (1H, dd), 2.67-2.53 (1H, m), 2.27-2.11 (1H, m), 2.06-1.89 (1H, m), 1.40 (3H, d), | carbaldehyde in Step 1. |
| | 1.04-0.83 (4H, m), 0.81-0.60 (2H, m), 0.41-0.22 (2H, m). | |
| 36 | 1H NMR (400 MHz, Me-d3-OD): 7.54-7.42 (1H, m), 7.42-7.26 (3H, m), 7.12 (1H, t), 6.95 (2H, d), | As Example 5/6 using Cyclopropyl methyl |
| | 4.66 (1H, dd), 2.67-2.53 (1H, m), 2.27-2.11 (1H, m), 2.06-1.89 (1H, m), 1.40 (3H, d), | ketone in Step 1. |
| | 1.04-0.83 (4H, m), 0.81-0.60 (2H, m), 0.41-0.22 (2H, m). | |
| 37 | 1H NMR (400 MHz, Me-d3-OD): 8.98 (1H, s), 7.78 (1H, s), 7.67-7.56 (1H, m), 7.43-7.31 (3H, m), | As Example 5/6 using Imidazole-4- |
| | 7.13 (1H, t), 6.98 (2H, d), 4.64 (1H, dd), 4.47 (1H, d), 4.32 (1H, d), 2.39-2.28 (1H, m), | carbaldehyde in Step 1. |
| | 2.21-2.09 (1H, m), 0.92 (3H, t). | |
| 38 | 1H NMR (400 MHz, Me-d3-OD): 7.70-7.59 (2H, m), 7.43-7.30 (3H, m), 7.13 (1H, t), 6.98 (2H, d), | As Example 5/6 using 2-Ethylimidazole-4-carbaldehyde |
| | 4.64 (1H, dd), 4.41 (1H, d), 4.26 (1H, d), 3.03 (2H, q), 2.41-2.28 (1H, m), 2.22-2.08 (1H, m), | in Step 1. |
| | 1.43 (3H, t), 0.92 (3H, t). | |
| 39 | 1H NMR (400 MHz, Me-d3-OD): 7.46-7.39 (1H, m), 7.39-7.31 (3H, m), 7.13 (1H, t), 6.96 (2H, d), | Example 39 |
| | 5.95-5.88 (1H, m), 5.55-5.45 (2H, m), 4.48 (1H, dd), 3.68-3.56 (2H, m), 2.24-2.17 (1H, m), | |
| | 2.07-1.99 (1H, m), 0.90 (3H, t). [M + H]+ 304 | |
| 40 | 1H NMR (400 MHz, DMSO-d6): 8.74 (3H, s), 7.51-7.43 (2H, m), 7.43-7.33 (2H, m), 7.14 (1H, t), | As Example 13, Step 1 using 1- |
| | 6.97 (2H, d), 5.86 (1H, s), 5.02 (1H, s), 2.13-1.94 (2H, m), 1.94-1.75 (2H, m), 1.66-1.43 (4H, m). | cyclohexene boronic acid. |
| 41 | 1H NMR (400 MHz, DMSO-d6): 7.45 (1H, q), 7.41-7.33 (2H, m), 7.32-7.24 (1H, m), 7.16-7.05 (1H, | As Example 13 using 3,6-Dihydro-2H- |
| | m), 6.97-6.87 (2H, m), 3.93-3.72 (3H, m), 3.27-3.08 (2H, m), 1.85-1.75 (1H, m), 1.75-1.57 (1H, m), | pyran-4-boronic acid pinacol ester in step |
| | 1.30-1.09 (3H, m). [M + H − NH3]+ 303 | 1. |
| 42 | 1H NMR (400 MHz, Me-d3-OD): 7.51-7.42 (1H, m), 7.42-7.29 (3H, m), 7.13 (1H, t), 6.96 (2H, d), | Example 42 |
| | 4.52 (1H, dd), 3.68-3.58 (2H, m), 3.40 (3H, s), 3.27-3.06 (2H, m), 2.28-2.17 (1H, m), | |
| | 2.11-1.99 (1H, m), 0.98-0.84 (3H, m). [M + H]+ 322 | |
| 43 | 1H NMR (400 MHz, Me-d3-OD): 7.55-7.48 (1H, m), 7.40-7.29 (3H, m), 7.12 (1H, t), 6.95 (2H, d), | As Example 5/6 using 1,3- |
| | 4.78-4.72 (1H, m), 3.83-3.69 (4H, m), 3.18-3.11 (1H, m), 2.33-2.17 (1H, m), 2.14-2.04 (1H, m), | Dihydroxyacetone in Step 1. |
| | 0.90 (3H, t). | |
| | [M + H]+ 338 | |
| 44 | 1H NMR (400 MHz, Me-d3-OD): 7.46-7.30 (4H, m), 7.13 (1H, t), 6.96 (2H, d), 4.38 (1H, dd), | As Example 5/6 using Cyclobutanone in |
| | 3.78-3.69 (1H, m), 2.34-2.20 (2H, m), 2.20-1.98 (4H, m), 1.97-1.83 (2H, m), 0.88 (3H, t). | Step 1. |
| | [M + H]+ 318 | |
| 45 | 1H NMR (400 MHz, Me-d3-OD): 7.54-7.44 (1H, m), 7.44-7.28 (3H, m), 7.12 (1H, t), 6.96 (2H, d), | Example 45 |
| | 4.85 (19H, s), 4.54 (1H, dd), 3.79 (2H, t), 3.19-3.09 (1H, m), 3.09-2.96 (1H, m), 2.31-2.18 (1H, m), | |
| | 0.91 (3H, t). [M + H − HOCH2CH2NH2]+ 247 | |
| 46 | 1H NMR (400 MHz, DMSO-d6): 8.66 (3H, s), 7.68-7.56 (1H, m), 7.51-7.41 (1H, m), 7.38 (2H, t), | Example 46 |
| | 7.14 (1H, t), 6.98 (2H, d), 4.50 (1H, s), 3.77 (2H, d). [M + H − NH3]+ 249 | |
| 48 | 1H NMR (400 MHz, Me-d3-OD): 7.53-7.42 (1H, m), 7.41-7.29 (3H, m), 7.13 (1H, t), 6.96 (2H, d), | As Example 5/6 using N-Methyl |
| | 4.65 (1H, dd), 3.71-3.60 (1H, m), 2.77 (3H, s), 2.68-2.47 (2H, m), 2.30-2.18 (1H, m), | acetoacetamide in Step 1. |
| | 2.13-2.00 (1H, m), 1.35 (3H, d), 0.93 (3H, t). [M + H]+ 363 | |
| 49 | 1H NMR (400 MHz, Me-d3-OD): 7.53-7.43 (1H, m), 7.41-7.28 (3H, m), 7.12 (1H, t), 6.97 (2H, d), | As Example 48 |
| | 4.64 (1H, dd), 3.51-3.40 (1H, m), 2.73 (3H, s), 2.61-2.51 (2H, m), 2.23-2.06 (2H, m), 1.37 (3H, d), | |
| | 0.92 (3H, t). | |
| | [M + H]+ 363 | |
| 50 | 1H NMR (400 MHz, Me-d3-OD): 7.54-7.42 (1H, m), 7.42-7.26 (3H, m), 7.13 (1H, t), 7.00 (2H, d), | Example 50 |
| | 5.38 (1H, s), 3.75-3.63 (1H, m), 3.47-3.37 (1H, m), 3.20-3.01 (2H, m). [M + H]+ 304 | |
| 51 | 1H NMR (400 MHz, Me-d3-OD): 7.42-7.25 (3H, m), 7.20-7.03 (2H, m), 6.90 (2H, d), 4.10-3.96 (1H, | As Example 5/6 using Methoxyacetone in |
| | m), 3.30-3.18 (5H, m), 2.81-2.71 (0.7H, m), 2.70-2.60 (0.3H, m), 1.95-1.81 (1H, m), 1.75-1.58 (1H, | Step 1. |
| | m), 0.98 (3H, d), 0.89-0.78 (3H, m). [M + H]+ 336 | |
| 52 | 1H NMR (400 MHz, DMSO-d6): 8.65 (3H, s), 7.70-7.58 (1H, m), 7.54-7.43 (1H, m), 7.43-7.33 (2H, | Key Intermediate 1 |
| | m), 7.14 (1H, t), 6.97 (2H, d), 4.39 (1H, s), 2.10-1.96 (1H, m), 1.93-1.79 (1H, m), 0.81 (3H, t). | |
| 53 | 1H NMR (400 MHz, Me-d3-OD): 7.49 (1H, d), 7.35 (3H, t), 7.12 (1H, t), 6.96 (2H, d), | Example 53 |
| | 4.58-4.46 (1H, m), 4.07-3.95 (1H, m), 3.09-2.86 (1.7H, m), 2.69 (0.3H, dd), 2.31-2.17 (1H, m), 2.13-1.99 (1H, | |
| | m), 1.32 (0.5H, dd), 1.20 (2.5H, dd), 0.95-0.83 (3H, m). [M + H]+ 322 | |
| 54 | ¹H NMR (400 MHz, DMSO-d₆): 9.92 (1H, s), 9.43 (1H, s), 7.88 (1H, s), 7.82-7.73 (1H, m), | Example 54 |
| | 7.68 (1H, d), 7.60 (1H, s), 7.38 (2H, t), 7.13 (1H, t), 6.92 (2H, d), 4.35 (1H, s), 3.75 (1H, s), 2.21 (1H, s), | |
| | 2.07-1.94 (1H, m), 1.41 (3H, d), 0.69 (3H, t). [M + H]+ 351 | |
| 55 | 1H NMR (400 MHz, Me-d3-OD): 7.50-7.39 (1H, m), 7.39-7.24 (3H, m), 7.16-7.06 (1H, m), | Example 55 |
| | 6.95 (2H, d), 4.57-4.18 (2H, m), 4.13-3.96 (2H, m), 3.94-3.62 (1H, m), 3.62-3.53 (1H, m), 2.04 (2H, d), | |
| | 0.95-0.83 (3H, m). | |
| 56 | 1H NMR (400 MHz, Me-d3-OD): 7.52-7.42 (1H, m), 7.42-7.28 (3H, m), 7.12 (1H, t), 6.95 (2H, d), | Example 56 |
| | 4.51 (1H, dd), 3.73-3.62 (2H, m), 3.22-2.98 (2H, m), 2.28-2.15 (1H, m), 2.13-1.99 (1H, m), | |
| | 1.97-1.83 (2H, m), 0.91 (3H, t). [M + H]+ 322 | |
| 57 | 1H NMR (400 MHz, Me-d3-OD): 7.43-7.20 (3H, m), 7.20-6.96 (2H, m), 6.90 (2H, d), 4.03 (1H, dd), | As Example 5/6 using Hydroxyacetone in |
| | 3.54-3.36 (2H, m), 2.73-2.57 (1H, m), 1.96-1.55 (2H, m), 0.97 (3H, d), 0.91-0.66 (3H, m). | Step 1. Separation of diastereoisomers by |
| | column chromatography. | |
| 58 | 1H NMR (400 MHz, Me-d3-OD): 7.42-7.22 (3H, m), 7.22-7.11 (1H, m), 7.07 (1H, t), 6.90 (2H, d), | As Example 57 |
| | 4.09-3.90 (1H, m), 3.80-3.40 (1H, m), 3.40-3.33 (1H, m), 2.59-2.48 (1H, m), 1.99-1.81 (1H, m), | |
| | 1.81-1.59 (1H, m), 0.98 (3H, d), 0.84 (3H, t). | |
| 59A | [M + H]+ 313 | Example 59, step 1. |
| 59B | 1H NMR (400 MHz, DMSO-d6): 9.30 (1H, br s), 9.12 (1H, br s), 9.02 (3H, br s), 7.74-7.64 (1H, m), | Example 59, step 2 |
| | 7.51 (1H, t), 7.44-7.34 (2H, m), 7.14 (1H, t), 7.00 (2H, d), 4.56-4.47 (1H, m), 3.55-3.44 (1H, m), | |
| | 3.19-3.09 (1H, m), 2.92-2.71 (2H, m), 2.49-2.41 (1H, m), 1.80-1.69 (1H, m), 1.65-1.55 (1H, m), | |
| | 1.55-1.44 (1H, m), 1.22-1.07 (1H, m). [M + H]+ 319 | |
| 60 | 1H NMR (400 MHz, DMSO-d6): 9.10 (1H, br s), 8.96 (3H, br s), 8.80 (1H, br s), 7.74-7.65 (1H, m), | As Example 59. |
| | 7.52 (1H, t), 7.39 (2H, t), 7.20-7.09 (1H, m), 7.07-6.97 (2H, m), 4.36 (1H, s), 3.26-3.16 (1H, m), | |
| | 2.92-2.83 (1H, m), 2.78-2.65 (1H, m), 2.65-2.54 (2H, m), 2.12-2.02 (1H, m), 1.94-1.83 (1H, m), | |
| | 1.71-1.61 (1H, m), 1.43-1.32 (1H, m). [M + H]+ 319 | |
| 61 | 1H NMR (400 MHz, DMSO-d6): 8.72 (3H, s), 7.69-7.59 (1H, m), 7.46 (1H, t), 7.42-7.33 (2H, m), | Example 61 |
| | 7.19-7.09 (1H, m), 6.98 (2H, d), 4.43 (1H, d), 3.89-3.78 (1H, m), 3.78-3.68 (1H, m), 3.53-3.48 (2H, | |
| | m), 3.23-3.12 (1H, m), 2.97-2.85 (1H, m), 2.19-2.08 (1H, m), 2.01-1.88 (1H, m). [M + H − NH3]+ 289 | |
| 62 | 1H NMR (400 MHz, DMSO-d6): 8.71 (3H, s), 7.72-7.62 (1H, m), 7.48 (1H, t), 7.39 (2H, t), | As Example 61 |
| | 7.19-7.09 (1H, m), 6.98 (2H, d), 4.47-4.36 (1H, m), 3.85 (1H, dd), 3.82-3.71 (2H, m), 3.61-3.55 (1H, m), | |
| | 2.94-2.82 (1H, m), 1.80-1.68 (1H, m), 1.43-1.32 (1H, m). [M + H − NH3]+ 289 | |
| 64 | 1H NMR (400 MHz, Me-d3-OD): 7.53-7.23 (3H, m), 7.23-6.98 (2H, m), 6.90 (2H, d), 4.27-3.84 (1H, | As Example 5/6 using Hydroxyacetone in |
| | m), 3.60-3.38 (2H, m), 2.73-2.46 (1H, m), 1.97-1.57 (2H, m), 0.97 (3H, d), 0.83 (3H, t). | Step 1. Separation of diastereoisomers by |
| | | column chromatography. |
| 65 | 1H NMR (400 MHz, Me-d3-OD): 7.42-7.23 (3H, m), 7.23-7.00 (2H, m), 6.90 (2H, d), 4.16-3.99 (1H, | As Example 64 |
| | m), 3.49-3.39 (1H, m), 3.35 (1H, s), 2.53 (1H, dd), 1.98-1.63 (2H, m), 0.98 (3H, d), 0.84 (3H, t). | |
| 66 | 1H NMR (400 MHz, Me-d3-OD): 7.54-7.43 (1H, m), 7.42-7.29 (3H, m), 7.12 (1H, t), 6.96 (2H, d), | As Example 5/6, using acetoacetamide in |
| | 4.65 (1H, dd), 3.73-3.57 (1H, m), 2.71-2.52 (2H, m), 2.29-2.16 (1H, m), 2.15-2.02 (1H, m), | step 1 |
| | 1.45-1.29 (3H, m), 1.00-0.87 (3H, m). [M + H]+ 349 | |
| 67 | ¹H NMR (400 MHz, Me-d₃-OD): 8.99 (1H, s), 7.82 (1H, s), 7.71 (1H, s), 7.64-7.48 (1H, m), | As Example 5/6, using 4-acetylimidazole |
| | 7.44-7.31 (3H, m), 7.19-7.08 (1H, m), 7.02-6.91 (2H, m), 4.69-4.53 (1H, m), 4.44 (1H, dd), | in step 1 |
| | 2.36-2.19 (1H, m), 2.19-2.01 (1H, m), 1.81 (3H, d), 0.94-0.79 (3H, m). [M + H]+ 358 | |
| 68 | 1H NMR (400 MHz, Me-d3-OD): 7.58-7.47 (1H, m), 7.43-7.31 (3H, m), 7.14 (1H, t), 6.96 (2H, d), | As Example 5/6, using N-Boc-3- |
| | 4.49 (1H, dd), 4.06-3.92 (2H, m), 2.95-2.83 (1H, m), 2.74-2.61 (1H, m), 2.61-2.41 (2H, m), | azetidinone in step 1. |
| | 2.29-2.17 (1H, m), 2.17-2.04 (1H, m), 0.90 (3H, t). [M + H]+ 333 | |
| 69 | 1H NMR (400 MHz, Me-d3-OD): 7.58-7.44 (1H, m), 7.41-7.28 (3H, m), 7.12 (1H, t), 6.95 (2H, d), | As Example 5/6, using L-Glyceraldehyde |
| | 4.82-4.39 (1H, m), 3.98-3.69 (2H, m), 3.66-3.43 (1H, m), 3.19-2.80 (2H, m), 2.32-2.17 (1H, m), | in step 1 |
| | 2.15-1.99 (1H, m), 0.99-0.84 (3H, m). [M + H]+ 338 | |
| 70 | 1H NMR (400 MHz, Me-d3-OD): 7.53-7.43 (1H, m), 7.41-7.29 (3H, m), 7.12 (1H, t), 6.96 (2H, d), | As Example 5/6, using acetoacetamide in |
| | 4.72-4.57 (1H, m), 3.69-3.58 (1H, m), 2.71-2.52 (2H, m), 2.29-2.18 (1H, m), 2.11-1.99 (1H, m), | step 1. Separation of diastereomers by |
| | 1.37 (3H, d), 0.93 (3H, t). [M + H]+ 349 | column chromatography. |
| 71 | 1H NMR (400 MHz, Me-d3-OD): 7.53-7.40 (1H, m), 7.40-7.28 (3H, m), 7.12 (1H, t), 6.96 (2H, d), | As Example 53 using 3- |
| | 4.52 (1H, dd), 3.31-3.16 (2H, m), 2.67 (2H, t), 2.29-2.15 (1H, m), 2.15-1.98 (1H, m), 0.93 (3H, t). | bromopropionamide |
| | [M + H]+ 335 | |
| 72A | [M + H − NH₃]+ 310 | Example 72, step 1. |
| 72B | 1H NMR (400 MHz, DMSO-d6): 8.77 (5H, br s), 7.76-7.67 (1H, m), 7.48 (1H, t), 7.43-7.31 (2H, m), | Example 72, step 2. |
| | 7.19-7.06 (1H, m), 6.99 (2H, d), 4.60-4.49 (1H, m), 3.27-3.13 (2H, m), 2.84-2.65 (2H, m), | |
| | 2.02-1.82 (2H, m), 1.82-1.69 (2H, m), 1.50-1.28 (3H, m). [M + H − NH3]+ 316 | |
| 73 | [M + H]⁺ 329. | Example 73, step 2. |
| 74A | 1H NMR (400 MHz, Me-d3-OD): 7.48-7.39 (1H, m), 7.39-7.27 (3H, m), 7.13 (1H, t), 6.97 (2H, d), | Example 73, step 3. |
| | 4.51 (1H, d), 2.98 (2H, d), 2.60-2.36 (3H, m), 2.26-2.15 (1H, m), 1.88-1.75 (1H, m). | |
| 74B | 1H NMR (400 MHz, Me-d3-OD): 7.49-7.40 (1H, m), 7.40-7.28 (3H, m), 7.13 (1H, t), 6.97 (2H, d), | Example 73, step 3. |
| | 4.56 (1H, d), 3.63-3.47 (1H, m), 3.26 (1H, dd), 2.58-2.43 (1H, m), 2.43-2.26 (2H, m), | |
| | 1.73-1.49 (2H, m). | |
| 75 | 1H NMR (400 MHz, Me-d3-OD): 7.66-7.49 (1H, m), 7.47-7.31 (3H, m), 7.13 (1H, t), 6.97 (2H, d), | Example 75. |
| | 4.77-4.63 (1H, m), 3.84-3.73 (1H, m), 3.73-3.60 (1H, m), 3.53 (1H, d), 3.48-3.39 (1H, m), | |
| | 3.26-3.17 (1H, m), 3.17-2.93 (2H, m), 2.65-2.48 (1H, m), 2.44-2.28 (1H, m), 1.80-1.58 (2H, m), | |
| | 1.58-1.41 (1H, m), 1.31 (3H, d). [M + H]+ 377 | |
| 76 | 1H NMR (400 MHz, Me-d3-OD): 7.68-7.51 (1H, m), 7.45-7.31 (3H, m), 7.13 (1H, t), 6.97 (2H, d), | As Example 75 |
| | 4.77-4.65 (1H, m), 3.77 (1H, dd), 3.69-3.56 (1H, m), 3.56-3.47 (1H, m), 3.47-3.38 (1H, m), | |
| | 3.17-2.92 (3H, m), 2.63-2.46 (1H, m), 2.46-2.29 (1H, m), 1.82-1.70 (1H, m), 1.70-1.42 (2H, m), | |
| | 1.38 (3H, d). [M + H]+ 377 | |
| 77 | 1H NMR (400 MHz, DMSO-d6): 8.60 (2H, d), 7.70-7.56 (2H, m), 7.38 (2H, t), 7.12 (1H, t), | Prepared in analogous manner to Key |
| | 6.91 (2H, d), 4.41 (1H, s), 2.08-1.95 (1H, m), 1.92-1.79 (1H, m), 0.82 (3H, t). [M + H − NH₃]+ 263 | Intermediate 1, starting with 6-chloro-2- |
| | | fluoro-3-methyl phenol. |
| 78 | 1H NMR (400 MHz, DMSO-d6): 8.58 (2H, s), 7.70-7.55 (2H, m), 7.38 (2H, dd), 7.12 (1H, t), | As Example 77 except using (R)-tert- |
| | 6.91 (2H, d), 4.41 (1H, s), 2.07-1.95 (1H, m), 1.92-1.79 (1H, m), 0.82 (3H, t). [M + H − NH₃]+ 263 | butylsulfinimide |
| 79 | 1H NMR (400 MHz, Me-d3-OD): 7.58 (1H, dd), 7.47 (1H, dd), 7.40-7.30 (2H, m), 7.11 (1H, t), | Example 79 |
| | 6.90 (2H, d), 4.65 (1H, dd), 3.69-3.60 (1H, m), 2.61 (2H, ddd), 2.27-2.19 (1H, m), 2.09-2.01 (1H, m), | |
| | 1.37 (3H, d), 0.93 (3H, t). [M + H]+ 365 | |
| 80 | 1H NMR (400 MHz, Me-d3-OD): 7.57 (1H, d), 7.52-7.43 (1H, m), 7.35 (2H, t), 7.11 (1H, t), | Example 79 |
| | 6.90 (2H, d), 4.64 (1H, dd), 3.47-3.41 (1H, m), 2.68-2.53 (2H, m), 2.22-2.04 (2H, m), 1.37 (3H, d), | |
| | 0.92 (3H, t). [M + H]+ 365 | |
| 81 | 1H NMR (400 MHz, Me-d3-OD): 7.58 (1H, dd), 7.54-7.43 (1H, m), 7.40-7.29 (2H, m), 7.11 (1H, t), | As Example 79 using (S)-1-(4-chloro-2- |
| | 6.89 (2H, d), 4.64 (1H, d), 3.82-3.71 (1H, m), 3.61-3.52 (1H, m), 3.30-3.20 (1H, m), 2.28-2.16 (1H, | fluoro-3-phenoxy- |
| | m), 2.12-1.97 (1H, m), 1.32 (3H, dd), 0.89 (3H, t). [M + H]+ 338 | phenyl)-propylamine and hydroxyacetone |
| 82 | 1H NMR (400 MHz, Me-d3-OD): 7.44-7.36 (1H, m), 7.36-7.27 (2H, m), 7.15 (1H, dt), | As Example 72 using (S)-(−)-2-methyl-2-propane sulfinimide. |
| | 7.11-7.03 (1H, m), 6.92 (2H, d), 4.28 (1H, t), 3.20-3.09 (2H, m), 2.77-2.60 (2H, m), 1.90-1.75 (2H, m), | |
| | 1.75-1.59 (2H, m), 1.56-1.41 (1H, m), 1.36-1.19 (2H, m). | |
| 83 | 1H NMR (400 MHz, Me-d3-OD): 7.58 (1H, dd), 7.52-7.42 (1H, m), 7.40-7.30 (2H, m), 7.11 (1H, t), | Example 88, first eluting isomer |
| | 6.90 (2H, d), 4.66 (1H, dd), 3.70-3.59 (1H, m), 2.72-2.51 (2H, m), 2.29-2.18 (1H, m), | |
| | 2.12-1.98 (1H, m), 1.37 (3H, d), 0.93 (3H, t). [M + H]+ 365 | |
| 84 | 1H NMR (400 MHz, Me-d3-OD): 7.41-7.28 (3H, m), 7.22-7.12 (1H, m), 7.12-7.04 (1H, m), | As Example 72 using (S)-(−)-2-Methyl-2- |
| | 6.91 (2H, d), 4.24 (1H, dd), 3.21-3.10 (2H, m), 2.88-2.77 (1H, m), 2.77-2.59 (2H, m), 2.25 (2H, d), | propane sulfinimide. Followed by |
| | 1.93-1.82 (1H, m), 1.82-1.59 (3H, m), 1.50-1.36 (1H, m), 1.36-1.21 (2H, m), 1.07 (3H, d). | treatment with acetoacetamide as |
| | | described in Example 5/6, step 1. |
| | | Separation of diastereomers by |
| | | preparative hplc |
| 85 | 1H NMR (400 MHz, Me-d3-OD): 7.43-7.27 (3H, m), 7.15 (1H, dt), 7.08 (1H, t), 6.91 (2H, d), | As Example 72 using (S)-(−)-2-methyl-2- |
| | 4.20 (1H, t), 3.11 (2H, d), 2.99-2.88 (1H, m), 2.71-2.55 (2H, m), 2.32 (1H, dd), 2.24 (1H, dd), 1.83 (1H, | propane sulfinimide. Followed by |
| | d), 1.73 (2H, dd), 1.66-1.53 (1H, m), 1.51-1.38 (1H, m), 1.32-1.16 (2H, m), 1.05 (3H, d). | treatment with acetoacetamide as |
| | | described in Example 79 |
| 87A | [M + H]⁺ 371. | Example 87, Step1 |
| 87B | 1H NMR (400 MHz, Me-d3-OD): 7.65-7.53 (1H, m), 7.43-7.31 (3H, m), 7.13 (1H, t), 6.98 (2H, d), | Example 87 |
| | 4.78 (1H, dd), 3.86-3.75 (2H, m), 3.46-3.37 (2H, m), 3.22-3.10 (1H, m), 3.08-2.98 (1H, m), | |
| | 2.98-2.81 (2H, m), 2.27-2.08 (2H, m), 2.03-1.94 (1H, m), 1.89-1.77 (1H, m), 1.61-1.41 (3H, m). | |
| 88 | 1H NMR (400 MHz, Me-d3-OD): 7.62-7.44 (2H, m), 7.40-7.29 (2H, m), 7.11 (1H, t), 6.90 (2H, d), | Example 88, second eluting isomer |
| | 4.65 (1H, dd), 3.54-3.39 (1H, m), 2.71-2.53 (2H, m), 2.27-2.01 (2H, m), 1.37 (3H, d), 0.91 (3H, t). | |
| | [M + H]⁺ 365.. | |
| 89 | 1H NMR (400 MHz, Me-d3-OD): 7.67-7.56 (1H, m), 7.44-7.31 (3H, m), 7.14 (1H, t), 6.98 (2H, d), | As Example 87, using hydroxyacetone in |
| | 3.78 (1H, dd), 3.61 (1H, dd), 3.38 (2H, d), 3.31-3.16 (1H, m), 3.00-2.81 (2H, m), 2.30-2.09 (2H, m), | step 1. Separation of diastereomers by reparative hplc. |
| | 2.09-1.96 (1H, m), 1.85-1.74 (1H, m), 1.61-1.38 (4H, m), 1.33 (3H, d) | |
| 90 | 1H NMR (400 MHz, DMSO-d6): 9.53 (2H, s), 8.77 (1H, s), 8.66 (1H, s), 7.98 (1H, q), 7.53 (1H, t), | As Example 89 |
| | 7.38 (2H, t), 7.14 (1H, t), 7.00 (2H, d), 4.73-4.63 (1H, m), 3.76-3.44 (5H, m), 3.18 (2H, dd), | |
| | 3.05 (1H, s), 2.83-2.75 (1H, m), 2.75-2.63 (1H, m), 2.15-2.01 (2H, m), 1.76 (1H, d), 1.60 (1H, d), | |
| | 1.44-1.26 (3H, m) | |
| 91 | 1H NMR (400 MHz, Me-d3-OD): 7.50-7.43 (1H, m), 7.41-7.30 (3H, m), 7.12 (1H, t), 6.97 (2H, d), | Example 91 |
| | 4.53 (1H, dd), 4.19 (2H, s), 3.45-3.37 (2H, m), 3.30-3.17 (2H, m), 2.75-2.66 (2H, m), | |
| | 2.26-2.18 (1H, m), 2.12-2.00 (1H, m), 0.93 (3H, t). [M + H]+ 374 | |
| 92 | 1H NMR (400 MHz, Me-d3-OD): 7.54-7.42 (1H, m), 7.41-7.28 (3H, m), 7.12 (1H, t), 6.96 (2H, d), | Example 92 |
| | 4.53 (1H, dd), 3.61 (2H, t), 3.32-3.12 (4H, m), 2.66 (2H, t), 2.30-2.16 (1H, m), 2.16-2.00 (1H, m), | |
| | 0.93 (3H, t). [M + H]+ 379 | |
| 93 | 1H NMR (400 MHz, Me-d3-OD): 7.56-7.46 (1H, m), 7.42-7.28 (3H, m), 7.13 (1H, t), 6.97 (2H, d), | As Example 72 using (R)-(+)-2-Methyl-2- |
| | 4.74 (1H, dd), 3.40 (2H, d), 3.03-2.86 (2H, m), 2.15-1.98 (3H, m), 1.98-1.87 (1H, m), | propane sulfinimide. |
| | 1.63-1.43 (3H, m) | |
| 94 | 1H NMR (400 MHz, DMSO-d6): 8.80 (3H, br s), 8.16 (3H, br s), 7.72-7.61 (2H, m), 7.39 (2H, t), | Made using methods described herein |
| | 7.15 (1H, t), 6.97 (2H, d), 4.55 (1H, dd), 2.89-2.76 (1H, m), 2.68-2.56 (1H, m), 2.42-2.30 (1H, m), | |
| | 2.30-2.18 (1H, m). | |
| 95A | MS: [M + H]⁺ 385 | Example 95, step 1. |
| 95B | NMR (400 MHz, Me-d3-OD): 7.68-7.57 (1H, m), 7.44-7.32 (3H, m), 7.14 (1H, t), 6.98 (2H, d), | Example 95 |
| | 3.80 (1H, dd), 3.60 (1H, dd), 3.39-3.36 (1H, m), 3.29-3.21 (1H, m), 2.99-2.81 (2H, m), 2.24-2.07 (2H, | |
| | m), 2.07-1.97 (1H, m), 1.86-1.76 (1H, m), 1.59-1.41 (3H, m), 1.34 (3H, d). [M + H]+ 391 | |
| 96 | 1H NMR (400 MHz, DMSO-d6): 8.85 (3H, br s), 7.94 (1H, q), 7.55 (1H, t), 7.38 (2H, t), 7.14 (1H, t), | Example 95 |
| | 7.00 (2H, d), 5.37 (1H, s), 4.66 (1H, s), 3.64-3.53 (2H, m), 3.22-3.17 (1H, m), 2.98 (1H, s), | |
| | 2.83-2.72 (1H, m), 2.72-2.64 (1H, m), 1.99-1.89 (1H, m), 1.75 (1H, d), 1.59 (1H, d), 1.43-1.22 (4H, m), | |
| | 1.18 (3H, d). [M + H]+ 3911H | |
| 97 | 1H NMR (400 MHz, Me-d3-OD): 7.57 (1H, dd), 7.45 (1H, dd), 7.39-7.29 (2H, m), 7.11 (1H, t), | Example 97 |
| | 6.90 (2H, d), 4.53 (1H, dd), 3.31-3.12 (2H, m), 2.66 (2H, t), 2.27-2.15 (1H, m), 2.13-1.98 (1H, m), | |
| | 0.93 (3H, t). [M + H]+ 351 | |
| 98 | 1H NMR (400 MHz, DMSO-d6): 10.03-9.95 (1H, s), 9.60 (1H, s), 8.75 (1H, s), 8.58 (1H, d), | As Example 87, using acetoacetamide in |
| | 7.97-7.89 (1H, m), 7.67 (1H, s), 7.54 (1H, t), 7.37 (2H, t), 7.18-7.09 (2H, m), 7.00 (2H, d), | step 1. Separation of diastereomers by |
| | 4.67-4.58 (1H, m), 3.26-3.11 (2H, m), 2.82-2.65 (2H, m), 2.57 (2H, d), 2.46-2.35 (1H, m), 2.14-2.03 (2H, m), 1.78 (1H, | prep hplc. |
| | m), 1.63 (1H, m), 1.35 (3H, m), 1.25 (3H, d). | |
| 99 | 1H NMR (400 MHz, Me-d3-OD): 7.59 (1H, s), 7.36 (3H, t), 7.13 (1H, t), 6.97 (2H, d), | As Example 98 |
| | 4.99-4.72 (1H, m), 3.54-3.19 (3H, m), 2.99-2.83 (2H, m), 2.64 (2H, d), 2.18 (2H, d), 2.07-1.97 (1H, m), | |
| | 1.86-1.76 (1H, m), 1.51 (3H, s), 1.40 (3H, d) | |
| 100 | 1H NMR (400 MHz, Me-d3-OD): 7.51-7.43 (1H, m), 7.40-7.30 (3H, m), 7.12 (1H, t), 6.96 (2H, d), | Example 100 |
| | 4.53 (1H, dd), 3.84-3.73 (2H, m), 3.17-3.09 (1H, m), 3.07-2.95 (1H, m), 2.30-2.17 (1H, m), | |
| | 2.13-1.98 (1H, m), 0.90 (3H, t). [M + H]+ 308 | |
| 101 | 1H NMR (400 MHz, Me-d3-OD): 7.64-7.50 (1H, m), 7.37 (3H, t), 7.14 (1H, t), 6.98 (2H, d), | As Example 87, using (tert- |
| | 4.82-4.72 (1H, m), 3.86-3.76 (2H, m), 3.45-3.28 (2H, m), 3.21-3.10 (1H, m), 3.07-2.98 (1H, m), | butyldimethylsiloxy)-acetaldehyde in step |
| | 2.98-2.82 (2H, m), 2.27-2.11 (2H, m), 2.04-1.95 (1H, m), 1.89-1.78 (1H, m), 1.61-1.45 (3H, m). | 1, followed by TBAF deprotection as in |
| | | Example 56. |
| 102 | 1H NMR (400 MHz, Me-d3-OD): 7.49-7.41 (1H, m), 7.41-7.29 (3H, m), 7.13 (1H, t), 6.96 (2H, d), | Example 102 |
| | 6.00-5.85 (1H, m), 5.52 (1H, s), 5.48 (1H, d), 4.49 (1H, dd), 3.72-3.54 (2H, m), 2.28-2.15 (1H, m), | |
| | 2.11-1.96 (1H, m), 0.90 (3H, t). [M + H]+ 304 | |
| 103 | 1H NMR (400 MHz, Me-d3-OD): 7.52-7.44 (1H, m), 7.40-7.32 (3H, m), 7.13 (1H, t), 6.96 (2H, d), | Example 103 |
| | 4.54 (1H, dd), 3.30-3.20 (1H, m), 3.14-3.04 (1H, m), 2.89-2.72 (2H, m), 2.32-2.16 (1H, m), | |
| | 2.16-2.00 (1H, m), 0.91 (3H, t). [M + H]+ 324 | |
| 104 | 1H NMR (400 MHz, Me-d3-OD): 7.56-7.43 (1H, m), 7.43-7.28 (3H, m), 7.13 (1H, t), 6.95 (2H, d), | Example 104 |
| | 4.71 (1H, dd), 3.56-3.44 (1H, m), 2.80-2.67 (1H, m), 2.44-2.28 (2H, m), 2.25 (3H, s), | |
| | 2.13-1.99 (2H, m), 1.84 (2H, d), 1.52-1.11 (4H, m), 0.90 (3H, t). | |
| 105A | MS: [M + H − NH3]⁺ = 318 | Example 105, step 3. |
| 105B | 1H NMR (400 MHz, Me-d3-OD): 7.45-7.37 (1H, m), 7.33 (3H, t), 7.07 (1H, t), 6.87 (2H, d), | Example 105 |
| | 4.70 (1H, dd), 3.38 (2H, t), 3.02-2.82 (2H, m), 2.66 (2H, q), 2.15-1.93 (4H, m), 1.89 (1H, d), | |
| | 1.60-1.40 (3H, m), 1.18 (3H, t). [Adduct] + 385 | |
| 106 | ¹H NMR (400 MHz, Me-d3-OD): 8.06 (1H, d), 7.86-7.73 (2H, m), 7.60 (1H, dd), 7.50 (1H, t), | Example 106 |
| | 4.53 (1H, dd), 2.25 (3H, s), 2.17-2.02 (3H, m), 0.98 (3H, t) | |
| 107 | 1H NMR (400 MHz, Me-d3-OD): 7.52 (2H, d), 7.40-7.29 (2H, m), 7.11 (1H, t), 6.91 (2H, d), | Example 107 |
| | 4.42 (2H, s), 3.82-3.67 (1H, m), 3.59-3.45 (2H, m), 3.09 (2H, t), 2.74 (2H, d), 1.47 (3H, d). | |
| 108A | MS: [M − H]⁻ 418 | Example 108 |
| 108B | 1H NMR (400 MHz, DMSO-d6): 8.53 (4H, br s), 7.73-7.62 (1H, m), 7.46 (1H, t), 7.32 (1H, t), | Example 108 |
| | 7.02-6.93 (1H, m), 6.90 (1H, d), 6.71-6.62 (1H, m), 5.76 (1H, s), 4.48 (1H, t), 3.26-3.14 (2H, m), | |
| | 3.01 (3H, s), 2.85-2.66 (2H, m), 1.95-1.70 (4H, m), 1.58-1.42 (1H, m), 1.42-1.24 (2H, m). [M + H]+ 426 | |
| 109 | 1H NMR (400 MHz, Me-d3-OD): 7.53-7.41 (1H, m), 7.41-7.30 (3H, m), 7.12 (1H, t), 6.96 (2H, d), | As Example 45, Step 1 using Key |
| | 4.53 (1H, dd), 3.22-3.08 (1H, m), 3.07-2.93 (1H, m), 2.61 (2H, t), 2.29-2.14 (1H, m), 2.14-1.94 (3H, | Intermediate 1 and 4-bromobutyronitrile. |
| | m), 0.91 (3H, t). [M + H]+ 331 | |
| 110 | ¹H NMR (400 MHz, Me-d3-OD): 8.12 (2H, s), 7.34 (3H, t), 7.17 (1H, t), 7.10 (1H, t), 6.99 (2H, d), | Example 110 |
| | 6.93 (2H, d), 4.65 (2H, s). MS: [M + H]+ 313 | |
| 111 | 1H NMR (400 MHz, DMSO-d6): 8.64 (3H, s), 7.69-7.59 (2H, m), 7.30 (1H, t), 7.05 (1H, d), | Example 111 |
| | 6.88 (1H, s), 6.75 (1H, dd), 5.25 (1H, t), 4.47 (2H, d), 4.40 (1H, t), 2.08-1.95 (1H, m), 1.91-1.77 (1H, m), | |
| | 0.81 (3H, t). [M + H]+ 310 | |
| 112 | 1H NMR (400 MHz, DMSO-d6): 8.68 (3H, s), 7.68-7.58 (2H, m), 7.26 (1H, s), 6.88 (1H, s), | Example 112 |
| | 6.77 (1H, d), 4.39 (1H, s), 2.28 (3H, s), 2.08-1.96 (1H, m), 1.95-1.80 (1H, m), 0.81 (4H, t). [M + H]+ 309 | |
| 113 | 1H NMR (400 MHz, Me-d3-OD): 8.08 (2H, d), 7.39-7.25 (3H, m), 7.22-7.13 (1H, m), 7.10 (1H, t), | Example 113 |
| | 6.95-6.82 (4H, m), 2.12-1.94 (2H, m), 1.05 (3H, t). [M + H]+ 341 | |
| 114 | 1H NMR (400 MHz, DMSO-d6): 8.74 (3H, s), 7.92-7.76 (2H, m), 7.76-7.54 (3H, m), 6.99 (1H, d), | As Example 112 using 5-chloro-2- |
| | 4.44-4.33 (1H, m), 2.09-1.97 (1H, m), 1.93-1.81 (1H, m), 0.82 (3H, t). | nitropyridine and the enantiomer of Key |
| | | Intermediate 3 in step 1. |
| 115 | 1H NMR (400 MHz, DMSO-d6): 8.63 (3H, s), 7.69-7.57 (2H, m), 7.31 (1H, t), 7.05 (1H, d), | As Example 111 using Key Intermediate |
| | 6.88 (1H, s), 6.75 (1H, dd), 5.25 (1H, t), 4.47 (2H, d), 4.44-4.34 (1H, m), 2.09-1.95 (1H, m), | 3. |
| | 1.92-1.78 (1H, m), 0.81 (3H, t). | |
| 116 | 1H NMR (400 MHz, DMSO-d6): 9.70 (1H, s), 9.31 (1H, s), 7.82-7.73 (1H, m), 7.73-7.64 (2H, m), | As Example 111 using Key Intermediate |
| | 7.36-7.25 (1H, m), 7.17 (1H, s), 7.05 (1H, d), 6.86 (1H, s), 6.76 (1H, d), 5.22 (1H, s), | 3, followed by reductive amination with |
| | 4.58-4.50 (1H, m), 4.46 (2H, s), 3.28-3.19 (1H, m), 2.70-2.57 (1H, m), 2.48-2.39 (1H, m), 2.24-2.12 (1H, m), | acetoacetamide as Example 79. |
| | 2.05-1.93 (1H, m), 1.20 (3H, d), 0.75 (3H, t). | Separation of diastereomers by preparative hplc. |
| 117 | 1H NMR (400 MHz, DMSO-d6): 9.74 (1H, s), 9.46 (1H, s), 7.87-7.76 (1H, m), 7.74-7.64 (2H, m), | As Example 116. |
| | 7.36-7.24 (1H, m), 7.17 (1H, d), 7.10-7.00 (1H, m), 6.88 (1H, s), 6.76 (1H, dd), 5.22 (1H, s), | |
| | 4.59-4.49 (1H, m), 4.47 (2H, s), 2.55 (1H, dd), 2.44-2.32 (1H, m), 2.25-2.13 (1H, m), 2.03-1.91 (1H, m), | |
| | 1.25 (3H, d), 0.76 (3H, t). | |
| 118 | 1H NMR (400 MHz, Me-d3-OD): 7.53 (1H, dd), 7.46-7.29 (3H, m), 7.11 (1H, t), 6.89 (2H, d), | As Key Intermediate 1 using 6-chloro-2- |
| | 4.58 (1H, t), 2.07-1.93 (2H, m), 1.44-1.22 (2H, m), 0.99 (3H, t). | fluoro-3-methylphenol in step 1 and n- |
| | [M + H]+ 294 | propyl magnesium bromide in step 5. |
| 119 | 1H NMR (400 MHz, Me-d3-OD): 7.53 (1H, dd), 7.46-7.29 (3H, m), 7.11 (1H, t), 6.89 (2H, d), | As Example 118 |
| | 4.58 (1H, dd), 2.05-1.94 (2H, m), 1.43-1.25 (2H, m), 0.99 (3H, t). | |
| | [M + H]+ 294 | |
| 120 | 1H NMR (400 MHz, Me-d3-OD): 7.42-7.26 (3H, m), 7.20-7.02 (2H, m), 6.91 (2H, d), 4.13-3.96 (1H, | As Example 79 using the enantiomer of |
| | m), 3.03-2.89 (1H, m), 2.35 (1H, dd), 2.24 (1H, dd), 1.94-1.78 (1H, m), 1.78-1.58 (1H, m), | Key Intermediate 1. Separation of |
| | 1.07 (3H, d), 0.86 (3H, t). | diastereomers by preparative hplc. |
| | [M + H]+ 349 | |
| 121 | 1H NMR (400 MHz, Me-d3-OD): 7.39-7.27 (3H, m), 7.20-7.02 (2H, m), 6.90 (2H, d), 4.05 (1H, dd), | As Example 79 using the enantiomer of |
| | 3.06-2.70 (1H, m), 2.35 (1H, dd), 2.32-2.19 (2H, m), 1.94-1.80 (1H, m), 1.75-1.60 (1H, m), | Key Intermediate 1. Separation of |
| | 1.14-1.02 (3H, m), 0.85 (3H, t) | diastereomers by preparative hplc. |
| | [M + H]+ 349 | |
| 122 | 1H NMR (400 MHz, Me-d3-OD): 7.52-7.41 (1H, m), 7.41-7.29 (3H, m), 7.12 (1H, t), 6.96 (2H, d), | As Example 53 using the enantiomer of |
| | 4.52 (1H, dd), 3.28-3.12 (2H, m), 2.67 (2H, t), 2.28-2.16 (1H, m), 2.15-2.02 (1H, m), 0.93 (3H, t). | Key Intermediate 1 and 3- |
| | | bromopropionamide |
| 123 | 1H NMR (400 MHz, Me-d3-OD): 7.93 (1H, dd), 7.68-7.54 (3H, m), 7.11 (1H, d), 4.68 (1H, dd), | As Example 112 using 5-chloro-2- |
| | 3.70-3.59 (1H, m), 2.66 (2H, d), 2.32-2.20 (1H, m), 2.14-1.97 (1H, m), 1.41 (3H, d), 0.93 (3H, t). | nitropyridine in step 1, followed by |
| | | reductive amination with acetoacetamide |
| | | as Example 5/6, step 1. Separation of |
| | | diastereomers by preparative hplc. |
| 124 | 1H NMR (400 MHz, Me-d3-OD): 7.93 (1H, dd), 7.69-7.52 (3H, m), 7.11 (1H, d), 4.65 (1H, dd), | As Example 112 using 5-chloro-2- |
| | 3.52-3.41 (1H, m), 2.71-2.59 (2H, m), 2.28-2.06 (2H, m), 1.40 (3H, d), 0.92 (3H, t). | nitropyridine in step 1, followed by |
| | | reductive amination with acetoacetamide |
| | | as Example 79. Separation of |
| | | diastereomers by preparative hplc. |
| 125 | 1H NMR (400 MHz, Me-d3-OD): 7.57 (1H, dd), 7.53-7.44 (1H, m), 7.35 (2H, t), 7.11 (1H, t), | As Example 79 using Example 118. |
| | 6.89 (2H, d), 4.76-4.65 (1H, m), 3.69-3.58 (1H, m), 2.68-2.53 (2H, m), 2.15-2.05 (2H, m), 1.37 (3H, d), | Separation of diastereomers by |
| | 1.28-1.20 (2H, m), 0.98 (3H, t). | preparative hplc |
| | [M + H]+ 379 | |
| 126 | 1H NMR (400 MHz, Me-d3-OD): 7.57 (1H, d), 7.52-7.43 (1H, m), 7.35 (2H, dd), 7.11 (1H, t), | As Example 125 |
| | 6.89 (2H, d), 4.69 (1H, s), 3.69-3.56 (1H, m), 2.64 (1H, d), 2.55 (1H, dd), 2.18-1.97 (2H, m), | |
| | 1.41-1.21 (5H, m), 0.98 (3H, t). M M + H]+ 379 | |
| 127 | 1H NMR (400 MHz, Me-d3-OD): 7.65-7.51 (2H, m), 7.36 (1H, d), 7.00 (1H, d), 6.88 (1H, dd), | As Example 112, followed by reductive |
| | 4.67 (1H, dd), 3.51-3.41 (1H, m), 2.65 (2H, dd), 2.40 (3H, s), 2.28-2.16 (1H, m), 2.15-2.05 (1H, m), | amination with acetoacetamide as |
| | 1.39 (3H, d), 0.92 (3H, t). | Example 79. Separation of diastereomers by preparative |
| | | hplc. |
| 128 | 1H NMR (400 MHz, Me-d3-OD): 7.64-7.49 (2H, m), 7.31 (1H, d), 6.97 (1H, d), 6.85 (1H, dd), | As Example 127 |
| | 4.67 (1H, dd), 3.71-3.60 (1H, m), 2.72-2.55 (2H, m), 2.39 (3H, s), 2.32-2.19 (1H, m), 2.13-2.01 (1H, m), | |
| | 1.39 (3H, d), 0.92 (3H, t). | |
| 129 | 1H NMR (400 MHz, Me-d3-OD): 7.60 (1H, s), 7.47-7.25 (4H, m), 7.07 (1H, t), 6.84 (2H, d), | As Example 79 using Example 78 and 4- |
| | 6.76 (1H, s), 3.75 (1H, s), 3.61 (1H, d), 1.85-1.71 (1H, m), 1.71-1.55 (1H, m), 1.35 (3H, d), | acetylimidazole. Separation of |
| | 0.88-0.71 (3H, m). | diastereomers by preparative hplc. |
| | [M + H]+ 374 | |
| 130 | 1H NMR (400 MHz, Me-d3-OD): 7.55 (1H, s), 7.41-7.27 (4H, m), 7.07 (1H, t), 6.90-6.77 (3H, m), | As Example 129 |
| | 4.00 (1H, dd), 3.77 (1H, d), 1.96-1.87 (1H, m), 1.72-1.62 (1H, m), 1.37 (3H, d), 0.81 (3H, t). | |
| | [M + H]+ 374 | |
| 131A | (400 MHz, Me-d₃-OD): 7.58 (1H, dd), 7.53-7.43 (1H, m), 7.35 (2H, t), 7.11 (1H, t), 6.90 (2H, d), | Example 131 |
| | 4.66 (1H, dd), 3.70-3.58 (1H, m), 2.76 (3H, s), 2.68-2.47 (2H, m), 2.30-2.17 (1H, m), | |
| | 2.13-1.99 (1H, m), 1.35 (3H, d), 0.93 (3H, t). [M + H]+ 366 | |
| 131B | 1H NMR (400 MHz, Me-d3-OD): 7.58 (1H, dd), 7.53-7.44 (1H, m), 7.35 (2H, t), 7.11 (1H, t), | Example 131 |
| | 6.91 (2H, d), 4.65 (1H, dd), 3.47-3.39 (1H, m), 2.73 (3H, s), 2.64-2.50 (2H, m), 2.26-2.04 (2H, m), | |
| | 1.36 (3H, d), 0.92 (3H, t). MS: [M + H]⁺ 366 | |
| 132A | [M + H]⁺ 322 | Example 132, step 3. |
| 132B | 1H NMR (400 MHz, DMSO-d6): 8.65 (2H, s), 7.70-7.57 (2H, m), 7.29 (1H, t), 7.07 (1H, dd), | Example 132 |
| | 6.92 (1H, s), 6.71 (1H, d), 5.21 (1H, d), 4.74-4.64 (1H, m), 4.40 (1H, dd), 2.09-1.96 (1H, m), | |
| | 1.91-1.77 (1H, m), 1.29 (3H, d), 0.80 (3H, t). [M + H]+ 322 | |
| 133 | 1H NMR (400 MHz, Me-d3-OD): 7.56 (1H, dd), 7.51-7.39 (2H, m), 7.29 (1H, d), 7.07 (1H, dd), | Example 133 |
| | 6.99 (1H, s), 4.51 (1H, dd), 3.58 (2H, t), 3.45 (2H, t), 2.17-1.96 (4H, m), 1.96-1.85 (2H, m), 0.97 (3H, t). | |
| | [M + H]+ 377 | |
| 134A | MS: [M + H]⁺ 361. | Example 134, Step 1 |
| 134B | 1H NMR (400 MHz, Me-d3-OD): 7.57 (1H, dd), 7.54-7.44 (1H, m), 7.34 (2H, t), 7.11 (1H, t), | Example 134 |
| | 6.90 (2H, d), 4.68 (1H, dd), 3.29-3.19 (1H, m), 2.74 (1H, dd), 2.53 (1H, dd), 2.26-2.16 (1H, m), | |
| | 2.16-2.04 (1H, m), 2.02-1.89 (1H, m), 1.71-1.58 (1H, m), 0.96 (3H, t), 0.91 (3H, t). [M + H]+ 379 | |
| 135 | 1H NMR (400 MHz, Me-d3-OD): 7.57 (1H, dd), 7.48 (1H, dd), 7.35 (2H, t), 7.11 (1H, t), 6.90 (2H, | Example 134 |
| | d), 4.80-4.62 (1H, m), 3.49-3.37 (1H, m), 2.77 (1H, dd), 2.54 (1H, dd), 2.30-2.17 (1H, m), | |
| | 2.13-1.99 (1H, m), 1.90-1.77 (1H, m), 1.73-1.60 (1H, m), 0.99 (3H, t), 0.93 (3H, t) [M + H]+ 379 | |
| 136 | 1H NMR (400 MHz, Me-d3-OD): 7.38-7.26 (3H, m), 7.17-7.02 (3H, m), 6.86 (2H, d), 6.69 (1H, d), | Example 136 |
| | 6.43 (1H, d), 4.67 (1H, t), 4.62-4.51 (4H, m), 2.05-2.00 (1H, m), 1.94-1.84 (1H, m), 1.04 (3H, t). | |
| | [M + H]+ 381 | |
| 137 | 1H NMR (400 MHz, Me-d3-OD): 5.88-5.75 (3H, m), 5.64-5.48 (3H, m), 5.36 (2H, d), 5.10 (1H, d), | As Example 136 using 2-Boc 8-bromo- |
| | 4.90 (1H, d), 3.16 (1H, t), 2.88-2.67 (2H, m), 2.04-1.91 (2H, m), 1.57 (2H, t), 0.63-0.48 (1H, m), | 1,2,3,4-tetrahydroisoquinoline in step 1. |
| | 0.47-0.34 (1H, m), −0.46 (3H, t). [M + H]+ 395 | |
| 138 | 1H NMR (400 MHz, DMSO-d6): 8.60 (2H, br s), 7.70-7.58 (2H, m), 7.45 (2H, t), 7.39-7.25 (4H, m), | Example 138 |
| | 7.25-7.18 (2H, m), 7.16 (1H, d), 7.05-6.97 (1H, m), 6.90 (1H, s), 5.30 (2H, s), 4.42 (2H, s), | |
| | 4.23 (1H, s), 2.80 (3H, s), 2.04-1.95 (1H, m), 1.88-1.79 (1H, m), 0.79 (3H, s). [M + H]+ 507 | |
| 139 | 1H NMR (400 MHz, Me-d3-OD): 7.54 (1H, d), 7.42 (1H, t), 7.31 (1H, t), 7.12 (1H, d), 6.92 (1H, s), | Example 139. |
| | 6.75 (1H, d), 5.73-5.62 (1H, m), 4.50 (1H, dd), 3.12 (2H, d), 2.16-1.95 (2H, m), 1.49 (3H, d), | |
| | 1.23-1.05 (3H, m), 0.97 (3H, t). [M + H]+ 395 | |
| 140 | 1H NMR (400 MHz, Me-d3-OD): 7.59-7.49 (1H, m), 7.42 (1H, t), 7.31 (1H, t), 7.11 (1H, d), | As Example 139. |
| | 6.92 (1H, s), 6.75 (1H, d), 5.73-5.62 (1H, m), 4.51 (1H, dd), 3.21-3.05 (2H, m), 2.16-1.95 (2H, m), | |
| | 1.49 (3H, d), 1.11 (3H, t), 0.97 (3H, t). | |
| 141 | 1H NMR (400 MHz, Me-d3-OD): 8.15 (1H, s), 7.75 (1H, s), 7.55 (1H, d), 7.51-7.41 (2H, m), | Example 141 |
| | 7.22 (1H, d), 7.03 (2H, s), 4.66 (1H, s), 4.57-4.42 (2H, m), 3.03 (3H, s), 2.16-2.06 (1H, m), | |
| | 2.06-1.96 (1H, m), 0.96 (3H, t). [M + H]+ 417 | |
| 142 | 1H NMR (400 MHz, Me-d3-OD): 7.58 (1H, dd), 7.48 (1H, dd), 7.41-7.29 (2H, m), 7.11 (1H, t), | As Example 134 using 4-methyl-3- |
| | 6.90 (2H, d), 4.70 (1H, dd), 3.45-3.36 (1H, m), 2.75 (1H, dd), 2.56 (1H, dd), 2.31-2.17 (1H, m), | oxopentanenitrile. |
| | 2.17-2.00 (2H, m), 1.09-0.84 (9H, m). | |
| 143 | 1H NMR (400 MHz, Me-d3-OD): 7.62-7.45 (2H, m), 7.40-7.29 (2H, m), 7.11 (1H, t), 6.90 (2H, d), | As Example 142 |
| | 4.70 (1H, dd), 3.41-3.27 (1H, m), 2.65 (1H, dd), 2.46 (1H, dd), 2.35-2.19 (2H, m), 2.19-2.04 (1H, | |
| | m), 1.03 (3H, d), 0.97 (3H, d), 0.91 (3H, t). | |
| 144 | 1H NMR (400 MHz, Me-d3-OD): 7.97 (2H, d), 7.40-7.30 (2H, m), 7.30-7.14 (1H, m), 7.14-7.04 (2H, | Example 144 |
| | m), 6.88 (2H, d), 6.51 (2H, d), 4.65 (1H, t), 2.04-1.71 (2H, m), 1.03 (3H, t). [M + H]+ 341 | |
| 145A | 400 MHz, Me-d₃-OD): 7.70 (2H, s), 7.60 (1H, dd), 7.44 (1H, dd), 7.41-7.32 (2H, m), 7.13 (1H, t), | Example 145 |
| | 6.92 (2H, d), 4.40 (1H, q), 4.24 (1H, dd), 2.16-2.05 (1H, m), 2.05-1.93 (1H, m), 1.70 (3H, d), | |
| | 0.81 (3H, t). MS: [M + H]⁺ 374. | |
| 145B | 1H NMR (400 MHz, Me-d3-OD): 7.81 (2H, s), 7.56 (1H, dd), 7.42 (1H, t), 7.38-7.29 (2H, m), | Example 145 |
| | 7.11 (1H, t), 6.88 (2H, d), 4.55 (1H, q), 4.40 (1H, dd), 2.33-2.19 (1H, m), 2.11-2.04 (1H, m), 1.70 (3H, | |
| | d), 0.85 (3H, t). [M + H]+ 374 | |
| 146 | 1H NMR (400 MHz, Me-d3-OD): 7.63-7.53 (1H, m), 7.48 (1H, t), 7.35 (2H, t), 7.11 (1H, t), | As Example 28 using ethanolamine in |
| | 6.90 (2H, d), 4.66 (1H, dd), 3.72-3.55 (3H, m), 3.37-3.34 (2H, m), 2.65 (1H, dd), 2.56 (1H, dd), | step 2. |
| | 2.31-2.16 (1H, m), 2.13-1.98 (1H, m), 1.36 (3H, d), 0.93 (3H, t). [M + H]+ 409 | |
| 147 | 1H NMR (400 MHz, Me-d3-OD): 7.57 (1H, d), 7.53-7.43 (1H, m), 7.34 (2H, t), 7.11 (1H, t), | As example 146 |
| | 6.90 (2H, d), 4.65 (1H, dd), 3.69-3.54 (2H, m), 3.49-3.40 (1H, m), 3.35-3.29 (2H, m), 2.68-2.52 (2H, m), | |
| | 2.26-2.01 (2H, m), 1.37 (3H, d), 0.92 (3H, t). [M + H]+ 409 | |
| 148 | 1H NMR (400 MHz, Me-d3-OD): 8.70 (1H, s), 7.52 (2H, s), 7.35 (2H, t), 7.11 (1H, t), 6.88 (2H, d), | As Example 79 using Example 78 and 4- |
| | 4.56 (1H, q), 4.45 (1H, dd), 2.25 (4H, s), 2.12-1.96 (1H, m), 1.73 (3H, d), 0.87 (3H, t). | acetyl-5-methylimidazole. |
| | [M + H]+ 388 | |
| 149 | 1H NMR (400 MHz, DMSO-d6): 8.66 (3H, s), 7.71-7.60 (2H, m), 7.42 (1H, q), 6.98 (1H, dt), | As Example 132, step 1 using Key |
| | 6.83 (1H, dt), 6.76 (1H, dd), 4.46-4.35 (1H, m), 2.09-1.96 (1H, m), 1.93-1.80 (1H, m), 0.82 (3H, t) | Intermediate 3 and 3-fluorophenyl boronic |
| | | acid followed by Key Intermediate 1, Step |
| | | 6. |
| 150 | 1H NMR (400 MHz, DMSO-d6): 8.53 (3H, s), 7.70-7.55 (2H, m), 7.21 (2H, t), 7.02-6.90 (2H, m), | As Example 132, step 1 using Key |
| | 4.40 (1H, dd), 2.06-1.94 (1H, m), 1.91-1.78 (1H, m), 0.81 (3H, t). | Intermediate 3 and 4-fluorophenyl boronic |
| | | acid followed by Key Intermediate 1, Step 6 |
| 151 | 1H NMR (400 MHz, DMSO-d6): 9.92 (1H, s), 8.58 (3H, s), 7.71-7.56 (2H, m), 7.31 (1H, t), | As Example 132, step 1 using Key |
| | 6.96 (1H, d), 6.83 (1H, s), 6.59 (1H, d), 4.41 (1H, s), 3.00 (3H, s), 2.06-1.93 (1H, m), 1.91-1.78 (1H, m), | Intermediate 3 and (3-methylsulfonyl- |
| | 0.81 (3H, t). | aminophenyl)boronic acid followed by Key |
| | | Intermediate 1, Step 6 |
| 152 | 1H NMR (400 MHz, DMSO-d6): 8.55 (3H, s), 8.02 (1H, d), 7.77-7.60 (4H, m), 7.52-7.43 (1H, m), | As Example 132, step 1 using Key |
| | 4.47-4.37 (1H, m), 2.07-1.94 (1H, m), 1.93-1.79 (1H, m), 0.89-0.75 (3H, m). | Intermediate 3 and 3-nitrophenyl boronic |
| | | acid followed by Key Intermediate 1, Step 6 |
| 153 | 1H NMR (400 MHz, DMSO-d6): 8.67 (3H, s), 8.11 (1H, d), 7.93 (1H, d), 7.68 (2H, s), 4.43 (1H, s), | As Example 112 using 3,6- |
| | 2.02 (1H, dd), 1.94-1.75 (1H, m), 0.80 (3H, d). [M + H]+ 316/318 | dichloropyrazine in step 1 followed by Key Intermediate 1, |
| | | Step 6. |
| 154 | 1H NMR (400 MHz, Me-d3-OD): 7.58 (2H, s), 7.35 (2H, t), 7.11 (1H, t), 6.89 (2H, d), 4.68 (1H, dd), | As Example 28 using hydrazine |
| | 3.55 (1H, dd), 2.85 (1H, dd), 2.75 (1H, dd), 2.31-2.05 (2H, m), 1.41 (3H, d), 0.91 (3H, t). [M + H]+ | dihydrochloride in step 2. |
| | 380 | |
| 155 | 1H NMR (400 MHz, Me-d3-OD): 7.58 (1H, d), 7.54-7.43 (1H, m), 7.35 (2H, t), 7.11 (1H, t), | As Example 28 using O- |
| | 6.90 (2H, d), 4.66 (1H, dd), 3.70 (3H, s), 3.57-3.44 (1H, m), 2.81-2.42 (2H, m), 2.27-2.03 (2H, m), | methylhydroxylamine hydrochloride in |
| | 1.43-1.34 (3H, m), 0.91 (3H, t). [M + H]+ 381 | step 2. |
| 156 | 1H NMR (400 MHz, DMSO-d6): 8.71 (3H, d), 7.85 (1H, s), 7.72-7.64 (1H, m), 7.64-7.54 (2H, m), | Example 156 |
| | 7.23 (1H, d), 4.39 (1H, s), 2.08-1.96 (1H, m), 1.89-1.75 (1H, m), 0.80 (3H, t). [M + H]+ 296 | |
| 157 | 1H NMR (400 MHz, Me-d3-OD): 7.54 (2H, d), 7.46-7.29 (4H, m), 7.13 (2H, d), 6.96 (2H, s), | As Example 132, step 1 using Key |
| | 6.84 (2H, d), 4.85 (55H, s), 4.51 (2H, t), 4.23 (3H, s), 3.68 (1H, s), 3.32 (84H, d), 2.87 (5H, s), | Intermediate 3 and (3-methylsulfonyl- |
| | 2.13-1.97 (4H, m), 1.03-0.91 (6H, m). [M + H]+ 387 | aminomethyl)benzene-boronic acid |
| | | followed by Key Intermediate 1, Step 6 |
| 158 | 1H NMR (400 MHz, Me-d3-OD): 7.76 (2H, d), 7.63-7.52 (1H, m), 7.48 (1H, t), 7.07 (2H, d), | As Example 132, step 1 using Key |
| | 4.85 (28H, s), 4.58-4.47 (1H, m), 3.40-3.22 (27H, m), 2.16-1.96 (2H, m), 0.98 (3H, t). [M + H]+ 305 | Intermediate 3 and 4-cyanophenyl-boronic |
| | | acid followed by Key Intermediate 1, Step 6 |
| 159 | 1H NMR (400 MHz, Me-d3-OD): 7.57 (1H, d), 7.52-7.39 (1H, m), 7.34 (2H, t), 7.10 (1H, t), | As Example 28 |
| | 6.90 (2H, d), 4.70-4.59 (1H, m), 3.96 (2H, s), 3.73 (3H, s), 3.55-3.43 (1H, m), 2.66 (2H, d), | |
| | 2.25-2.02 (2H, m), 1.39 (3H, d), 0.91 (3H, t). [M + H]+ 437 | |
| 160 | 1H NMR (400 MHz, Me-d3-OD): 7.59 (1H, d), 7.48 (1H, t), 7.35 (2H, t), 7.11 (1H, t), 6.90 (2H, d), | As Example 28, using N, O |
| | 4.68 (1H, dd), 3.78-3.62 (4H, m), 3.21 (3H, s), 2.97-2.79 (2H, m), 2.33-2.19 (1H, m), | dimethylhydroxylamine hydrochloride in |
| | 2.14-1.99 (1H, m), 1.38 (3H, d), 0.92 (3H, t). [M + H]+ 409 | step 2. |
| 161 | 1H NMR (400 MHz, Me-d3-OD): 7.58 (1H, d), 7.50 (1H, t), 7.34 (2H, t), 7.11 (1H, t), 6.92 (2H, d), | As Example 160 |
| | 4.67 (1H, dd), 3.75 (3H, s), 3.54-3.45 (1H, m), 3.20 (3H, s), 2.94 (1H, d), 2.84 (1H, dd), | |
| | 2.26-2.05 (2H, m), 1.42 (3H, d), 0.92 (3H, t). [M + H]+ 409 | |
| 162 | 1H NMR (400 MHz, DMSO-d6): 8.61 (2H, s), 7.75-7.57 (3H, m), 7.52 (1H, d), 7.31-7.16 (2H, m), | As Example 132, step 1 using Key |
| | 4.42 (1H, s), 2.08-1.95 (1H, m), 1.93-1.79 (1H, m), 0.88-0.72 (3H, m). [M + H]+ 348 | Intermediate 3 and 3- |
| | | trifluoromethylphenylboronic acid followed |
| | | by Key Intermediate 1, Step 6 |
| 163 | 1H NMR (400 MHz, Me-d3-OD): 7.42-7.30 (4H, m), 7.08 (1H, t), 6.85 (2H, d), 6.67 (1H, s), | Example 163 |
| | 6.46 (1H, s), 6.04 (1H, s), 3.84 (1H, d), 3.51 (1H, d), 1.85-1.72 (1H, m), 1.68-1.55 (1H, m), 1.33 (3H, d), | |
| | 0.74 (3H, t). [M + H]+ 373 | |
| 164 | 1H NMR (400 MHz, Me-d3-OD): 7.33 (4H, dd), 7.12-7.02 (1H, m), 6.84 (2H, d), 6.63 (2H, d), | As Example 163 |
| | 6.02 (1H, s), 4.05-3.96 (1H, m), 3.68 (1H, d), 1.95 (1H, t), 1.74-1.62 (1H, m), 1.33 (3H, d), | |
| | 0.85-0.70 (3H, m). | |
| | [M + H]+ 373 | |
| 165 | 1H NMR (400 MHz, Me-d3-OD): 7.60-7.53 (1H, m), 7.53-7.44 (1H, m), 7.35 (2H, t), 7.11 (1H, t), | As Example 134 using 3-cyclopropyl-3- |
| | 6.89 (2H, d), 3.55-3.45 (1H, m), 2.81-2.74 (2H, m), 2.30-2.18 (1H, m), 2.10-1.97 (1H, m), 1.20 (1H, | propionitrile. |
| | dt), 1.13-1.01 (1H, m), 0.92 (3H, t), 0.87-0.75 (1H, m), 0.75-0.63 (1H, m), 0.41-0.25 (2H, m). | |
| 166 | 1H NMR (400 MHz, Me-d3-OD): 7.79 (1H, t), 7.59 (1H, dd), 7.50 (1H, dd), 7.01-6.90 (2H, m), | As Example 132, step 1 using Key |
| | 4.51 (1H, t), 2.14-1.97 (2H, m), 0.98 (3H, t). | Intermediate 3 and 4-cyano-3- |
| | [M + H]+ 323 | fluorophenyl-boronic acid followed by Key |
| | | Intermediate 1, Step 6 |
| 167 | 1H NMR (400 MHz, Me-d3-OD): 8.33 (1H, d), 7.62 (1H, dd), 7.56 (1H, dd), 7.07-6.96 (2H, m), | As Example 132, step 1 using Key |
| | 4.55 (1H, dd), 2.20-1.96 (2H, m), 0.98 (3H, t). [M + H]+ 315 | Intermediate 3 and 2-chloropyridine-4- |
| | | boronic acid followed by Key Intermediate |
| | | 1, Step 6. |
| 168 | 1H NMR (400 MHz, Me-d3-OD): 8.70 (1H, s), 7.67 (2H, s), 7.56 (1H, s), 7.53-7.43 (1H, m), | As Example 132, step 1 using Key |
| | 4.64-4.51 (1H, m), 2.78 (3H, s), 2.23-1.95 (2H, m), 1.00 (3H, t). | Intermediate 3 and 2-methylpyridine-4- |
| | | boronic acid followed by Key Intermediate |
| | | 1, Step 6. |
| 169 | 1H NMR (400 MHz, Me-d3-OD): 7.85-7.59 (4H, m), 5.52 (1H, s), 4.58 (1H, dd), 2.20-1.99 (2H, m), | As Example 132, step 1 using Key |
| | 1.00 (3H, t). | Intermediate 3 and pyridine-4-boronic acid |
| | | followed by Key Intermediate 1, Step 6. |
| 170 | 1H NMR (400 MHz, DMSO-d6): 8.55 (2H, s), 7.77-7.60 (4H, m), 7.42 (1H, s), 7.30 (1H, d), | As Example 132, step 1 using Key |
| | 4.43 (1H, t), 3.26 (3H, s), 2.12-1.93 (1H, m), 1.90-1.79 (1H, m), 0.82 (3H, t). [M + H]+ 358 | Intermediate 3 and 3-methanesulfonyl- |
| | | phenyl boronic acid followed by Key |
| | | Intermediate 1, Step 6. |
| 171 | 1H NMR (400 MHz, Me-d3-OD): 7.53 (1H, dd), 7.44-7.35 (1H, m), 7.29 (1H, s), 7.23 (1H, t), | Example 171 |
| | 6.87 (1H, d), 6.58 (1H, dd), 4.50 (1H, dd), 3.88 (2H, q), 2.14-1.97 (2H, m), 0.97 (3H, t). [M + H]+ 420 | |
| 172 | 1H NMR (400 MHz, Me-d3-OD): 7.67-7.54 (2H, m), 7.53-7.32 (3H, m), 7.17 (1H, dd), 4.52 (1H, | As Example 132, step 1 using Key |
| | dd), 2.15-1.97 (2H, m), 0.98 (3H, t). M M + H]+ 323 | Intermediate 3 and 3-aminocarbonyl- |
| | | phenyl boronic acid followed by Key |
| | | Intermediate 1, Step 6. |
| 173 | 1H NMR (400 MHz, DMSO-d6): 8.62-8.49 (2H, m), 8.38 (1H, d), 7.66 (1H, d), 7.59 (1H, s), | As Example 132, step 1 using Key |
| | 7.30 (1H, t), 7.00 (1H, d), 6.85 (1H, s), 6.70 (1H, d), 4.41 (1H, s), 4.23 (2H, d), 2.04-1.96 (1H, m), | Intermediate 3 and (3-acetamidomethyl- |
| | 1.86 (4H, s), 0.82 (3H, t). [M + H]+ 351 | phenyl)-boronic acid followed by Key |
| | | Intermediate 1, Step 6. |
| 174 | 1H NMR (400 MHz, DMSO-d6): 8.54 (2H, d), 7.95 (2H, d), 7.71 (1H, d), 7.64 (1H, s), 7.17 (2H, d), | As Example 132, step 1 using Key |
| | 4.43 (1H, t), 3.22 (3H, s), 2.05-1.96 (1H, m), 1.91-1.81 (1H, m), 0.83 (3H, t). [M + H]+ 358 | Intermediate 3 and 4-methanesulfonyl- |
| | | phenyl boronic acid followed by Key |
| | | Intermediate 1, Step 6. |
| 175 | 1H NMR (400 MHz, DMSO-d6): 8.60-8.46 (2H, m), 7.78 (2H, d), 7.74-7.59 (2H, m), 7.13 (2H, d), | As Example 132, step 1 using Key |
| | 4.43 (1H, s), 2.04-1.95 (1H, m), 1.92-1.81 (1H, m), 0.82 (3H, t). [M + H]+ 348 | Intermediate 3 and 4-trifluoromethyl- |
| | | phenyl boronic acid followed by Key |
| | | Intermediate 1, Step 6. |
| 176 | 1H NMR (400 MHz, DMSO-d6): 8.65 (2H, s), 7.71-7.59 (2H, m), 7.48-7.39 (2H, m), 7.01-6.91 (2H, | As Example 132, step 1 using Key |
| | m), 4.40 (1H, s), 2.08-1.96 (1H, m), 1.92-1.79 (1H, m), 0.81 (3H, t). [M + H]+ 314 | Intermediate 3 and 4-chlorophenyl boronic |
| | | acid followed by Key Intermediate 1, Step |
| | | 6. |
| 177 | 1H NMR (400 MHz, Me-d3-OD): 7.58 (1H, dd), 7.54-7.47 (1H, m), 7.35 (2H, t), 7.11 (1H, t), | As Example 28, using glycinamide |
| | 6.90 (2H, d), 4.67 (1H, dd), 4.01-3.83 (2H, m), 3.75-3.60 (1H, m), 2.78-2.67 (1H, m), 2.67-2.58 (1H, m), | hydrochloride in step 2. |
| | 2.32-2.17 (1H, m), 2.12-1.96 (1H, m), 1.41-1.36 (3H, m), 0.91 (3H, t). [M + H]+ 422 | |
| 178 | 1H NMR (400 MHz, Me-d3-OD): 7.57 (1H, dd), 7.50 (1H, dd), 7.40-7.29 (2H, m), 7.11 (1H, t), | As Example 177 |
| | 6.90 (2H, d), 4.66 (1H, dd), 3.89 (2H, dd), 3.53-3.41 (1H, m), 2.75-2.58 (2H, m), 2.26-2.05 (2H, m), | |
| | 1.39 (3H, d), 0.91 (3H, t). [M + H]+ 422 | |
| 179 | 1H NMR (400 MHz, Me-d3-OD): 7.55 (1H, dd), 7.42 (1H, t), 7.36 (2H, d), 6.93 (2H, d), 4.51 (1H, | As Example 132, step 1 using Key |
| | dd), 3.88 (2H, s), 2.18-1.91 (2H, m), 0.97 (3H, t). [M + H]+ 319 | Intermediate 3 and (4-cyanomethylphenyl)boronic acid followed |
| | | by Key Intermediate |
| | | 1, Step 6. |
| 180 | 1H NMR (400 MHz, Me-d3-OD): 8.09 (1H, d), 7.41 (2H, d), 7.31-7.21 (2H, m), 4.10 (1H, t), | As Example 132, step 1 using Key |
| | 2.51 (3H, s), 1.86-1.68 (2H, m), 0.89 (3H, t). [M + H]+ 295 | Intermediate 3 and 6-methylpyridine-3- |
| | | boronic acid followed by Key Intermediate |
| | | 1, Step 6. |
| 181 | 1H NMR (400 MHz, Me-d3-OD): 8.06-7.98 (2H, m), 7.42 (2H, d), 7.11-7.02 (2H, m), 4.15-4.06 (1H, | As Example 132, step 1 using Key |
| | m), 2.62 (3H, s), 2.61 (1H, s), 1.90-1.68 (2H, m), 0.91 (3H, t). [M + H]+ 362 | Intermediate 3 and 4-(5-methyl-1,3,4- |
| | | oxadiazol-2-yl)-phenylboronic acid |
| | | followed by Key Intermediate 1, Step 6. |
| 182 | 1H NMR (400 MHz, Me-d3-OD): 7.37-7.27 (2H, m), 6.77 (4H, d), 4.07 (1H, t), 2.87 (6H, s), | As Example 132, step 1 using Key |
| | 1.85-1.67 (2H, m), 0.88 (3H, t). | Intermediate 3 and 4-(dimethylamino)- |
| | | phenylboronic acid followed by Key |
| | | Intermediate 1, Step 6. |
| 183 | 1H NMR (400 MHz, DMSO-d6): 8.63 (2H, s), 7.69-7.56 (2H, m), 7.30 (2H, d), 6.86 (2H, d), | As Example 111 starting from key |
| | 5.15 (1H, t), 4.45 (2H, d), 4.40 (1H, dd), 2.08-1.95 (1H, m), 1.92-1.78 (1H, m), 0.81 (3H, t). | intermediate 3, using (4- |
| | | hydroxymethylphenyl)boronic acid in |
| | | Step1 |
| 184 | 1H NMR (400 MHz, Me-d3-OD): 7.44 (1H, dd), 7.38 (1H, dd), 7.34-7.23 (2H, m), 6.91-6.81 (2H, | Example 184 |
| | m), 4.33 (1H, q), 4.26 (1H, t), 3.21 (3H, s), 1.94-1.78 (2H, m), 1.40 (3H, d), 0.92 (3H, t). [M + H]+ | |
| | 338 | |
| 185 | 1H NMR (400 MHz, Me-d3-OD): 7.82-7.72 (2H, m), 7.63 (1H, dd), 7.54 (1H, dd), 7.14-7.05 (2H, | As Example 79 using Example 158. |
| | m), 4.68 (1H, dd), 3.71-3.59 (1H, m), 2.72-2.51 (2H, m), 2.31-2.18 (1H, m), 2.13-1.99 (1H, m), | Separation of diastereomers by |
| | 1.38 (3H, d), 0.94 (3H, t). [M + H]+ 390 | preparative hplc. |
| 186 | 1H NMR (400 MHz, Me-d3-OD): 7.81-7.72 (2H, m), 7.66-7.50 (2H, m), 7.15-7.06 (2H, m), | As Example 185 |
| | 4.67 (1H, dd), 3.49-3.41 (1H, m), 2.70-2.52 (2H, m), 2.29-2.02 (2H, m), 1.43-1.34 (3H, m), | |
| | 0.99-0.87 (3H, m). | |
| | [M + H]+ 390 | |
| 187 | 1H NMR (400 MHz, Me-d3-OD): 7.52 (1H, dd), 7.43-7.33 (1H, m), 7.21-7.17 (1H, m), | As Example 132, step 1 using Key |
| | 7.17-7.12 (1H, m), 6.84-6.75 (2H, m), 4.50 (1H, dd), 3.54-3.47 (1H, m), 2.63 (2H, q), 2.14-1.95 (2H, m), | Intermediate 3 and 4-ethylphenylboronic |
| | 1.28-1.15 (3H, m), 0.97 (3H, t). [M + H]+ 308 | acid followed by Key Intermediate 1, Step 6 |
| 188 | 1H NMR (400 MHz, Me-d3-OD): 7.45 (1H, dd), 7.39 (1H, t), 7.33 (2H, d), 6.84 (2H, d), 4.82 (1H, | Example 184 |
| | q), 4.32 (1H, s), 2.04-1.79 (2H, m), 1.43 (3H, d), 0.92 (3H, t). [M + H]+ 324 | |
| 189 | 1H NMR (400 MHz, Me-d3-OD): 7.85 (2H, d), 7.58 (1H, d), 7.52-7.42 (1H, m), 7.09 (2H, d), | As Example 132, step 1 using Key |
| | 4.85 (27H, s), 4.53 (1H, t), 3.33 (41H, s), 2.55 (4H, s), 2.16-1.96 (3H, m), 1.45-1.28 (1H, m), | Intermediate 3 and 4-methylamino- |
| | 1.27-1.13 (2H, m), 1.03-0.90 (4H, m). [M + H]+ 373 | sulfonyl-phenylboronic acid followed by |
| | | Key Intermediate 1, Step 6 |
| 190 | 1H NMR (400 MHz, DMSO-d6): 8.61 (3H, s), 7.70-7.56 (2H, m), 7.39 (1H, t), 7.23 (1H, t), | Example 190 |
| | 7.10-7.00 (1H, m), 6.91-6.81 (1H, m), 5.03 (2H, s), 4.40 (1H, dd), 2.92-2.79 (2H, m), 2.07-1.95 (1H, m), | |
| | 1.91-1.78 (1H, m), 0.94-0.85 (1H, m), 0.82 (3H, t), 0.44-0.38 (1H, m), 0.37 (1H, d), 0.19-0.07 (2H, | |
| | m). [M + H]+ 425 | |
| 191 | 1H NMR (400 MHz, Me-d3-OD): 7.56 (1H, d), 7.51-7.32 (4H, m), 7.08 (1H, dd), 4.09 (1H, t), | As Example 132, step 1 using Key |
| | 3.61 (2H, t), 2.79 (2H, t), 1.89-1.66 (2H, m), 0.90 (3H, t). | Intermediate 3 and 3-(2-cyanoethyl- |
| | [M + H]+ 376 | aminocarbonyl)-benzene-boronic acid |
| | | followed by Key Intermediate 1, Step 6. |
| 192 | 1H NMR (400 MHz, DMSO-d6): 8.61 (2H, s), 7.70-7.57 (2H, m), 7.40-7.30 (2H, m), 6.98-6.88 (2H, | As Example 132, step 1 using Key |
| | m), 4.40 (1H, dd), 2.08-1.95 (1H, m), 1.92-1.78 (1H, m), 1.76-1.67 (2H, m), 1.51-1.41 (2H, m), | Intermediate 3 and 1-(4-borono-phenyl)- |
| | 0.81 (3H, t). | cyclo-propane carbo-nitrile followed by |
| | | Key Intermediate 1, Step 6. |
| 193 | 1H NMR (400 MHz, Me-d3-OD): 7.50 (1H, dd), 7.36 (1H, dd), 6.82 (1H, s), 6.69-6.56 (2H, m), | As Example 132, step 1 using Key |
| | 4.60-4.44 (3H, m), 3.18 (2H, t), 2.14-1.94 (2H, m), 0.96 (3H, t). [M + H]+ 322 | Intermediate 3 and 2,3-dihydro-1- |
| | | benzofuran-5-yl boronic acid followed by |
| | | Key Intermediate 1, Step 6. |
| 194 | 1H NMR (400 MHz, DMSO-d6): 8.62 (3H, s), 7.69-7.56 (2H, m), 7.33-7.23 (2H, m), 6.80 (2H, d), | As Example 132, step 1 using Key |
| | 4.65 (1H, t), 4.39 (1H, dd), 3.48 (2H, d), 2.08-1.95 (1H, m), 1.92-1.79 (1H, m), 0.93-0.75 (6H, m), | Intermediate 3 and 4-(1-(hydroxy- |
| | 0.75-0.63 (2H, m). | methyl)cyclo-propyl)-phenyl boronic acid |
| | | followed by Key Intermediate 1, Step 6. |
| 195 | 1H NMR (400 MHz, Me-d3-OD): 7.59 (2H, d), 7.52-7.44 (1H, m), 6.86 (1H, d), 6.43 (1H, dd), | As Example 132, step 1 using Key |
| | 4.53 (1H, dd), 3.95 (3H, s), 2.20-1.97 (2H, m), 0.97 (3H, t). [M + H]+ 335 | Intermediate 3 and 4-cyano-3- |
| | | methoxyphenyl boronic acid followed by |
| | | Key Intermediate 1, Step 6. |
| 196 | 1H NMR (400 MHz, DMSO-d6): 9.94-9.88 (1H, m), 9.55-9.34 (1H, m), 9.34-9.13 (1H, m), | As Example 88, using Example 151. |
| | 7.76-7.64 (3H, m), 7.35-7.26 (1H, m), 7.22-7.15 (1H, m), 6.95 (1H, d), 6.79 (1H, d), 6.62 (1H, dd), | |
| | 4.59-4.50 (1H, m), 3.29-3.20 (1H, m), 3.03-2.95 (3H, m), 2.63-2.53 (1H, m), 2.44 (1H, dd), | |
| | 2.20-2.10 (1H, m), 2.02-1.92 (1H, m), 1.30-1.16 (3H, m), 0.76 (3H, t). [M + H]+ 458/460 | |
| 197 | 1H NMR (400 MHz, DMSO-d6): 8.57 (3H, s), 8.34-8.25 (2H, m), 7.77-7.62 (2H, m), 7.22-7.13 (2H, | As Example 132, step 1 using Key |
| | m), 4.43 (1H, t), 2.07-1.95 (1H, m), 1.93-1.81 (1H, m), 0.83 (3H, t). | Intermediate 3 and 4-nitrophenyl boronic |
| | | acid followed by Key Intermediate 1, Step |
| | | 6. |
| 198 | 1H NMR (400 MHz, Me-d3-OD): 7.53 (1H, dd), 7.45-7.36 (1H, m), 7.28 (2H, d), 6.90-6.81 (2H, m), | As Example 132, step 1 using Key |
| | 4.50 (1H, dd), 4.33 (2H, s), 2.15-1.95 (5H, m), 0.96 (3H, t). [M + H]+ 351 | Intermediate 3 and (4-acetamidomethyl- |
| | | phenyl)-boronic acid followed by Key |
| | | Intermediate 1, Step 6. |
| 199 | 1H NMR (400 MHz, DMSO-d6): 8.59 (3H, s), 7.73-7.55 (4H, m), 7.45 (1H, s), 7.37-7.27 (1H, m), | As Example 132, step 1 using Key |
| | 4.41 (1H, dd), 2.09-1.93 (1H, m), 1.93-1.80 (1H, m), 0.83 (3H, t). | Intermediate 3 and 3-cyanophenyl-boronic |
| | | acid followed by Key Intermediate 1, Step |
| | | 6. |
| 200 | 1H NMR (400 MHz, Me-d3-OD): 7.64-7.55 (3H, m), 7.46 (1H, t), 7.29 (1H, s), 7.26-7.19 (1H, m), | As Example 132, step 1 using Key |
| | 4.55-4.48 (1H, m), 2.54 (3H, s), 2.13-1.97 (2H, m), 0.97 (3H, t). [M + H]+ 373 | Intermediate 3 and 3-methylamino- |
| | | sulfonyl-phenyl boronic acid followed by |
| | | Key Intermediate 1, Step 6. |
| 201 | 1H NMR (400 MHz, DMSO-d6): 9.52 (1H, s), 9.26-9.16 (1H, m), 8.33-8.24 (2H, m), 7.84-7.73 (2H, | As Example 88, using Example 197 |
| | m), 7.73-7.64 (1H, m), 7.27-7.16 (3H, m), 4.56 (1H, s), 3.28 (1H, s), 2.65-2.55 (1H, m), | |
| | 2.49-2.39 (1H, m), 2.21-2.10 (1H, m), 2.00 (1H, q), 1.23 (3H, d), 0.78 (3H, t). | |
| 202 | [M + H]+ 410/412 | As Example 201 |
| 203 | 1H NMR (400 MHz, DMSO-d6): 8.84-8.66 (3H, m), 8.42 (1H, s), 7.78-7.70 (2H, m), 7.70-7.65 (2H, | Example 203 |
| | m), 7.62 (1H, s), 7.04 (2H, d), 4.40 (1H, s), 2.11-1.98 (1H, m), 1.94-1.80 (1H, m), 0.82 (3H, t). | |
| | [M + H]+ 347 | |
| 204 | 1H NMR (400 MHz, DMSO-d6): 11.14 (1H, br s), 8.68 (3H, s), 8.11 (1H, s), 8.00 (1H, d), | Example 204 |
| | 7.72-7.63 (2H, m), 7.60 (1H, d), 6.96 (2H, dd), 4.46-4.35 (1H, m), 2.09-1.97 (1H, m), 1.93-1.81 (1H, m), | |
| | 0.82 (3H, t). [M + H]+ 323 | |
| 205 | 1H NMR (400 MHz, Me-d3-OD): 7.58 (1H, dd), 7.48 (1H, dd), 7.41-7.30 (2H, m), 7.11 (1H, t), | As Example 28 using (S)-2-amino-propan- |
| | 6.90 (2H, d), 4.66 (1H, dd), 4.08-3.94 (1H, m), 3.70-3.57 (1H, m), 3.52 (1H, dd), 3.47 (1H, dd), | 1-ol in step 2. |
| | 2.69-2.49 (2H, m), 2.31-2.17 (1H, m), 2.12-1.98 (1H, m), 1.36 (3H, d), 1.22-1.11 (3H, m), 0.93 (3H, t). | |
| | [M + H]+ 423 | |
| 206 | 1H NMR (400 MHz, Me-d3-OD): 7.57 (1H, dd), 7.49 (1H, dd), 7.40-7.29 (2H, m), 7.11 (1H, t), | As Example 205 |
| | 6.90 (2H, d), 4.65 (1H, dd), 4.03-3.92 (1H, m), 3.56-3.40 (3H, m), 2.67-2.50 (2H, m), 2.27-2.04 (2H, m), | |
| | 1.37 (3H, d), 1.20-1.08 (3H, m), 0.92 (3H, t). [M + H]+ 423 | |
| 207 | 1H NMR (400 MHz, Me-d3-OD): 7.57 (1H, dd), 7.49 (1H, dd), 7.40-7.29 (2H, m), 7.11 (1H, t), | As Example 28 using (R)-2-amino- |
| | 6.90 (2H, d), 4.65 (1H, dd), 4.03-3.91 (1H, m), 3.54-3.39 (3H, m), 2.66-2.49 (2H, m), 2.26-2.05 (2H, m), | propan-1-ol in step 2. |
| | 1.36 (3H, d), 1.14 (3H, d), 0.92 (3H, t). [M + H]+ 423 | |
| 208 | 1H NMR (400 MHz, Me-d3-OD): 7.58 (1H, dd), 7.48 (1H, dd), 7.41-7.29 (2H, m), 7.11 (1H, t), | As Example 207 |
| | 6.90 (2H, d), 4.66 (1H, dd), 4.07-3.94 (1H, m), 3.72-3.58 (1H, m), 3.58-3.43 (2H, m), 2.70-2.48 (2H, m), | |
| | 2.31-2.16 (1H, m), 2.14-1.97 (1H, m), 1.36 (3H, d), 1.15 (3H, d), 0.93 (3H, t). [M + H]+ 423 | |
| 209 | 1H NMR (400 MHz, Me-d3-OD): 7.67-7.50 (3H, m), 6.87 (1H, d), 6.46 (1H, dd), 4.68 (1H, dd), | As Example 132, step 1 using 4-cyano-3- |
| | 3.95 (3H, s), 3.69-3.60 (1H, m), 2.68-2.53 (2H, m), 2.29-2.19 (1H, m), 2.10-2.00 (1H, m), 1.37 (3H, d), | methoxyphenyl boronic acid. Then as |
| | 0.93 (3H, t). M M + H]+ 420 | Example 88. |
| 210 | 1H NMR (400 MHz, Me-d3-OD): 7.66-7.51 (3H, m), 6.85 (1H, d), 6.49 (1H, dd), 4.68 (1H, dd), | As Example 209. |
| | 3.98-3.88 (3H, m), 3.52-3.41 (1H, m), 2.70-2.53 (2H, m) 2.24-2.06 (2H, m), 1.38 (3H, d), 0.92 (3H, | |
| | t). | |
| | [M + H]+ 420 | |
| 211 | [1H NMR (400 MHz, Me-d3-OD): 7.62-7.55 (1H, m), 7.55-7.46 (1H, m), 7.36 (2H, d), | As Example 88 using Example 179. |
| | 6.98-6.90 (2H, m), 4.65 (1H, dd), 3.88 (2H, s), 3.48-3.39 (1H, m), 2.70-2.49 (2H, m), 2.30-1.98 (2H, m), | Separation of diastereomers by |
| | 1.38 (3H, d), 0.92 (3H, t). M M + H]+ 404 | preparative hplc. |
| 212 | 1H NMR (400 MHz, Me-d3-OD): 7.58 (1H, d), 7.52-7.46 (1H, m), 7.37 (2H, d), 6.94 (2H, d), | As Example 211 |
| | 4.68-4.62 (1H, m), 3.89 (2H, s), 3.69-3.54 (1H, m), 2.80-2.46 (2H, m), 2.30-2.15 (1H, m), 2.13-1.94 (1H, | |
| | m), 1.37 (3H, d), 0.93 (3H, t). M M + H]+ 404 | |
| 213 | 1H NMR (400 MHz, Me-d3-OD): 7.79 (1H, dd), 7.64 (1H, dd), 7.59 (1H, dd), 7.04 (1H, dd), | As Example 88 using Example 166. |
| | 6.95 (1H, dd), 4.68 (1H, dd), 3.72-3.60 (1H, m), 2.73-2.52 (2H, m), 2.32-2.19 (1H, m), 2.14-1.99 (1H, | Separation of diastereomers by |
| | m), 1.39 (3H, d), 0.94 (3H, t). | preparative hplc. |
| 214 | 1H NMR (400 MHz, Me-d3-OD): 7.79 (1H, dd), 7.68-7.54 (2H, m), 7.05 (1H, dd), 6.96 (1H, dd), | As Example 213 |
| | 4.67 (1H, dd), 3.53-3.40 (1H, m), 2.72-2.53 (2H, m), 2.28-2.05 (2H, m), 1.39 (3H, d), | |
| | 0.99-0.87 (3H, m). | |
| 215 | 1H NMR (400 MHz, Me-d3-OD): 7.55 (1H, dd), 7.51-7.39 (3H, m), 6.88 (2H, d), 4.50 (1H, dd), | As Example 132, step 1 using Key |
| | 3.48-3.41 (1H, m), 2.13-1.97 (2H, m), 0.97 (3H, t). [M + H]+ 304 | Intermediate 3 and using 4-(dihydroxy- |
| | | borophenyl)-acetylene followed by Key |
| | | Intermediate 1, Step 6. |
| 216 | 1H NMR (400 MHz, Me-d3-OD): 7.58 (1H, dd), 7.50 (1H, dd), 7.36 (1H, t), 7.10 (1H, d), 6.94 (1H, | As Example 88 using Example 157. |
| | dd), 6.87 (1H, s), 4.65 (1H, dd), 4.21 (2H, s), 3.47-3.38 (1H, m), 2.85 (3H, s), 2.67 (1H, dd), | Separation of diastereomers by |
| | 2.58 (1H, dd), 2.25-2.05 (2H, m), 1.38 (3H, d), 0.92 (3H, t). | preparative hplc. |
| 217 | 1H NMR (400 MHz, Me-d3-OD): 7.59 (1H, dd), 7.49 (1H, dd), 7.36 (1H, t), 7.12 (1H, d), | As Example 216 |
| | 6.96-6.86 (2H, m), 4.67 (1H, dd), 4.22 (2H, s), 3.66-3.56 (1H, m), 2.85 (3H, s), 2.65 (1H, dd), 2.60 (1H, dd), | |
| | 2.29-2.16 (1H, m), 2.12-2.03 (1H, m), 1.37 (3H, d), 0.94 (3H, t). | |
| 218 | 1H NMR (400 MHz, DMSO-d6): 8.66 (3H, d), 7.71-7.65 (2H, m), 7.47 (1H, d), 6.16 (1H, dd), | Example 218 |
| | 5.34 (1H, d), 4.45-4.35 (1H, m), 2.10-1.97 (1H, m), 1.94-1.80 (1H, m), 0.81 (3H, t). [M + H]+ 297 | |
| 219 | 1H NMR (400 MHz, Me-d3-OD): 8.25 (1H, s), 7.79-7.70 (2H, m), 7.60 (1H, dd), 7.53 (1H, dd), | As Example 88 using Example 203. |
| | 7.47 (1H, s), 7.03 (2H, d), 4.67 (1H, dd), 3.53-3.40 (1H, m), 2.71-2.54 (2H, m), 2.26-2.05 (2H, m), | Separation of diastereomers by |
| | 1.39 (3H, d), 0.93 (3H, t). | preparative hplc. |
| 220 | 1H NMR (400 MHz, Me-d3-OD): 8.27 (1H, s), 7.80-7.70 (2H, m), 7.61 (1H, dd), 7.53 (1H, dd), | As Example 219 |
| | 7.48 (1H, s), 7.07-6.98 (2H, m), 4.68 (1H, dd), 3.71-3.60 (1H, m), 2.66 (1H, dd), 2.59 (1H, dd), | |
| | 2.32-2.19 (1H, m), 2.14-1.99 (1H, m), 1.38 (3H, d), 0.94 (3H, t). | |
| 221 | 1H NMR (400 MHz, Me-d3-OD): 7.67 (1H, d), 7.58 (1H, dd), 7.52-7.42 (1H, m), 6.97 (1H, d), | As Example 132, step 1 using Key |
| | 6.85 (1H, dd), 4.52 (1H, dd), 2.52 (3H, s), 2.16-1.96 (2H, m), 0.98 (3H, t). | Intermediate 3 and 4-cyano-3-methyl- |
| | [M + H]+ 319 | phenyl boronic acid followed by Key |
| | | Intermediate 1, Step 6. |
| 222 | 1H NMR (400 MHz, Me-d3-OD): 7.63-7.54 (1H, m), 7.54-7.44 (1H, m), 7.41-7.29 (2H, m), | As Example 28 using oxetan-3-amine in |
| | 7.11 (1H, t), 6.90 (2H, d), 4.66 (1H, dd), 4.20-4.12 (1H, m), 3.78-3.58 (4H, m), 3.53-3.41 (1H, m), | step 2. |
| | 2.74-2.56 (2H, m), 2.28-2.05 (2H, m), 1.45-1.34 (3H, m), 0.92 (3H, t). [M + H]+ 421 | |
| 223 | 1H NMR (400 MHz, Me-d3-OD): 7.63-7.49 (2H, m), 7.41-7.29 (2H, m), 7.11 (1H, t), 6.90 (2H, d), | Example 223 |
| | 4.68 (1H, dd), 3.76-3.62 (1H, m), 3.58-3.41 (2H, m), 3.09 (2H, t), 2.70 (2H, dd), 2.36-2.20 (1H, m), | |
| | 2.16-2.03 (1H, m), 1.38 (3H, d), 0.92 (3H, t). [M + H]+ 408 | |
| 224 | 1H NMR (400 MHz, Me-d3-OD): 7.57 (2H, d), 7.35 (2H, dd), 7.11 (1H, t), 6.90 (2H, d), 4.67 (1H, | Example 223 |
| | dd), 3.55-3.42 (3H, m), 3.07 (2H, t), 2.80-2.61 (2H, m), 2.31-2.05 (2H, m), 1.45-1.35 (3H, m), | |
| | 0.91 (3H, t). [M + H]+ 408 | |
| 225A | MS: [M + H]+ 372. | Example 225 |
| 225B | 1H NMR (400 MHz, Me-d3-OD): 8.19 (1H, d), 8.08 (1H, d), 7.44-7.31 (2H, m), 7.26-7.15 (1H, m), | Example 225 |
| | 7.06-6.96 (3H, m), 6.92 (1H, dd), 4.66 (2H, s), 2.40 (3H, s). [M + H]+ 342.0 | |
| 226 | 1H NMR (400 MHz, DMSO-d6): 9.53-9.43 (1H, m), 9.23-9.14 (1H, m), 7.74 (2H, s), 7.70-7.52 (4H, | As Example 88 using Example 199. |
| | m), 7.31 (1H, d), 7.17 (1H, s), 4.54 (1H, s), 3.40-3.33 (1H, m), 2.64-2.55 (1H, m), 2.47-2.39 (1H, | |
| | m), 2.20-2.10 (1H, m), 2.05-1.94 (1H, m), 1.22 (3H, d), 0.77 (3H, t). | |
| 227 | 1H NMR (400 MHz, Me-d3-OD): 7.56 (1H, dd), 7.50-7.39 (1H, m), 7.34-7.18 (3H, m), 4.52 (1H, | Example 227 |
| | dd), 2.16-1.95 (2H, m), 0.97 (3H, t). [M + H]+ 341 | |
| 228 | 1H NMR (400 MHz, Me-d3-OD): 8.31 (1H, s), 7.67 (1H, d), 7.57 (1H, dd), 7.45 (1H, dd), 7.33 (1H, | As Example 203 using 4-formyl-3- |
| | s), 6.90 (1H, d), 6.82 (1H, dd), 4.53 (1H, dd), 2.46 (3H, s), 2.17-1.95 (2H, m), 0.98 (3H, t) | methylphenyl boronic acid in step 1 |
| 229 | 1H NMR (400 MHz, Me-d3-OD): 7.59 (2H, s), 7.46 (2H, d), 6.90 (2H, d), 4.72-4.60 (1H, m), | Example 229 |
| | 3.61 (1H, d), 3.45 (1H, s), 2.65 (2H, d), 2.34-2.18 (1H, m), 2.16-1.99 (1H, m), 1.39 (3H, d), 0.91 (3H, t). | |
| | [M + H]+ 389 | |
| 230 | 1H NMR (400 MHz, Me-d3-OD): 7.64-7.51 (2H, m), 7.46 (2H, d), 6.90 (2H, d), 4.65 (1H, dd), | As Example 229 |
| | 3.53-3.40 (2H, m), 2.67 (2H, d), 2.31-2.18 (1H, m), 2.18-2.03 (1H, m), 1.38 (3H, d), 0.91 (3H, t). [M + H]+ | |
| | 389 | |
| 231 | 1H NMR (400 MHz, Me-d3-OD): 8.61 (1H, d), 8.09 (1H, dd), 7.97 (1H, d), 7.64 (1H, dd), 7.57 (1H, | As Example 132, step 1 using Key |
| | dd), 4.95 (2H, s), 4.56 (1H, dd), 2.18-1.98 (2H, m), 0.99 (3H, t). | Intermediate 3 and 6-hydroxymethyl- |
| | | pyridine-3-boronic acid followed by Key |
| | | Intermediate 1, Step 6. |
| 232 | 1H NMR (400 MHz, Me-d3-OD): 7.55 (1H, dd), 7.42 (1H, dd), 6.84 (1H, s), 6.70-6.57 (2H, m), | As Example 88 using Example 193. |
| | 4.64 (1H, dd), 4.55 (2H, t), 3.69-3.59 (1H, m), 3.18 (2H, t), 2.71-2.51 (2H, m), 2.28-2.17 (1H, m), | Separation of diastereomers by |
| | 2.10-1.99 (1H, m), 1.36 (3H, d), 0.92 (3H, t). | preparative hplc. |
| | [M + H]+ 407 | |
| 233 | 1H NMR (400 MHz, Me-d3-OD): 7.54 (1H, dd), 7.43 (1H, dd), 6.84 (1H, s), 6.69-6.57 (2H, m), | As Example 232 |
| | 4.64 (1H, dd), 4.55 (2H, t), 3.46-3.41 (1H, m), 3.24-3.12 (2H, m), 2.69-2.53 (2H, m), 2.22-2.03 (2H, m), | |
| | 1.37 (3H, d), 0.91 (3H, t). | |
| | [M + H]+ 407 | |
| 234 | 1H NMR (400 MHz, Me-d3-OD): 7.62-7.51 (2H, m), 7.41-7.30 (2H, m), 7.12 (1H, t), 6.90 (2H, d), | As Example 28 using 1-Boc-3- |
| | 4.75-4.61 (2H, m), 4.35-4.23 (2H, m), 4.23-4.13 (2H, m), 3.56-3.44 (1H, m), 2.78-2.61 (2H, m), | aminoazetidine in step 2 followed by |
| | 2.32-2.04 (2H, m), 1.39 (3H, d), 0.91 (3H, t). [M + H]+ 420 | Example 223, Step 2. |
| 235 | 1H NMR (400 MHz, Me-d3-OD): 7.62-7.51 (2H, m), 7.40-7.29 (2H, m), 7.11 (1H, t), 6.91 (2H, d), | As Example 28 using N-Boc-piperazine in |
| | 4.67 (1H, dd), 3.92-3.72 (4H, m), 3.54-3.42 (1H, m), 3.37-3.17 (4H, m), 2.94 (1H, dd), 2.83 (1H, | step 2 followed by Example 223, Step 2. |
| | dd), 2.28-2.06 (2H, m), 1.43 (3H, d), 0.92 (3H, t). [M + H]+ 434 | |
| 236 | 1H NMR (400 MHz, Me-d3-OD): 7.62-7.55 (1H, m), 7.55-7.49 (1H, m), 7.34 (2H, t), 7.11 (1H, t), | As Example 28 using N-Boc- |
| | 6.91 (2H, d), 4.68 (1H, dd), 3.96-3.21 (7H, m), 2.91 (1H, dd), 2.86-2.73 (1H, m), 2.26-2.00 (4H, m), | homopiperazine in step 2 followed by |
| | 1.43 (3H, d), 0.92 (3H, t). [M + H]+ 448 | Example 223, Step 2. |
| 237 | 1H NMR (400 MHz, Me-d3-OD): 7.74-7.55 (3H, m), 6.99 (1H, s), 6.88 (1H, dd), 4.66 (1H, dd), | As Example 88 using Example 221. |
| | 3.52-3.42 (1H, m), 2.79-2.59 (2H, m), 2.51 (3H, s), 2.34-2.20 (1H, m), 2.20-2.02 (1H, m), 1.39 (3H, | Separation of diastereomers by |
| | d), 0.90 (3H, t). | preparative hplc. |
| | [M + H]+ 404 | |
| 238A | 1H NMR (400 MHz, Me-d3-OD): 7.67 (1H, dd), 7.64-7.55 (1H, m), 7.39 (1H, d), 7.01 (1H, d), | Example 238 |
| | 6.88 (1H, dd), 4.69 (1H, dd), 3.58-3.40 (4H, m), 3.09 (2H, t), 2.85-2.63 (2H, m), 2.41 (3H, s), | |
| | 2.33-2.06 (2H, m), 1.39 (3H, d), 0.91 (3H, t). | |
| | MS: [M + H]⁺ 339. | |
| 238B | 1H NMR (400 MHz, Me-d3-OD): 7.67 (1H, dd), 7.64-7.55 (1H, m), 7.39 (1H, d), 7.01 (1H, d), | Example 238 |
| | 6.88 (1H, dd), 4.69 (1H, dd), 3.58-3.40 (4H, m), 3.09 (2H, t), 2.85-2.63 (2H, m), 2.41 (3H, s), | |
| | 2.33-2.06 (2H, m), 1.39 (3H, d), 0.91 (3H, t). [M + H]+ 437 | |
| 239 | 1H NMR (400 MHz, Me-d3-OD): 7.69-7.55 (2H, m), 7.39 (1H, d), 7.01 (1H, d), 6.88 (1H, dd), | As Example 238 |
| | 4.70 (1H, dd), 3.80-3.69 (1H, m), 3.51 (2H, t), 3.11 (2H, t), 2.82-2.64 (2H, m), 2.41 (3H, s), | |
| | 2.38-2.22 (1H, m), 2.18-1.98 (1H, m), 1.40 (3H, d), 0.92 (3H, t). [M + H]+ 437 | |
| 240 | 1H NMR (400 MHz, Me-d3-OD): 7.63-7.48 (2H, m), 7.41-7.30 (2H, m), 7.11 (1H, t), 6.90 (2H, d), | As Example 28 using N-Boc-1,3- |
| | 4.65 (1H, dd), 3.53-3.41 (1H, m), 3.36-3.23 (2H, m), 2.96 (2H, t), 2.75-2.59 (2H, m), 2.29-2.04 (2H, | propanediamine in step 2 followed by |
| | m), 1.93-1.79 (2H, m), 1.37 (3H, d), 0.92 (3H, t). [M + H]+ 422 | Example 223, Step 2. |
| 241 | 1H NMR (400 MHz, Me-d3-OD): 7.63-7.54 (2H, m), 7.41-7.30 (2H, m), 7.11 (1H, t), 6.90 (2H, d), | As Example 28 using N-Boc-N-methyl- |
| | 4.67 (1H, dd), 3.60-3.41 (3H, m), 3.14 (2H, t), 2.80-2.61 (2H, m), 2.71 (3H, s), 2.29-2.08 (2H, m), | ethylenediamine in step 2 followed by |
| | 1.39 (3H, d), 0.91 (3H, t). [M + H]+ 422 | Example 223, Step 2. |
| 242 | 1H NMR (400 MHz, Me-d3-OD): 7.64-7.52 (2H, m), 7.41-7.30 (2H, m), 7.11 (1H, t), 6.91 (2H, d), | As Example 28 using N,N- |
| | 4.66 (1H, dd), 3.69-3.58 (1H, m), 3.58-3.45 (2H, m), 3.26 (2H, t), 2.91 (6H, s), 2.80-2.62 (2H, m), | dimethylethylenediamine in step 2 |
| | 2.28-2.09 (2H, m), 1.39 (3H, d), 0.91 (3H, t). [M + H]+ 436 | |
| 243 | 1H NMR (400 MHz, Me-d3-OD): 7.56 (1H, dd), 7.50 (1H, dd), 7.40-7.29 (2H, m), 7.11 (1H, t), | As Example 28 using 2-amino-N- |
| | 6.90 (2H, d), 4.66 (1H, dd), 3.87 (1H, d), 3.81 (1H, d), 3.53-3.42 (1H, m), 3.25 (2H, q), 2.75-2.57 (2H, | ethylacetamide in step 2 |
| | m), 2.26-2.05 (2H, m), 1.39 (3H, d), 1.14 (3H, t), 0.91 (3H, t). [M + H]+ 450 | |
| 244 | 1H NMR (400 MHz, Me-d3-OD): 7.57 (1H, dd), 7.49 (1H, dd), 7.25 (1H, d), 6.82 (1H, d), 6.69 (1H, | Example 244 |
| | dd), 4.65 (1H, dd), 3.48-3.38 (1H, m), 2.69-2.52 (2H, m), 2.28-2.01 (8H, m), 1.37 (3H, d), 0.91 (3H, | |
| | t). [M + H]+ 436 | |
| 245 | 1H NMR (400 MHz, Me-d3-OD): 7.64-7.47 (2H, m), 7.25 (1H, d), 6.83 (1H, d), 6.70 (1H, dd), | As Example 244 |
| | 4.66 (1H, dd), 3.70-3.57 (1H, m), 2.73-2.54 (2H, m), 2.35-2.20 (4H, m), 2.16 (3H, s), 2.12-1.96 (1H, m), | |
| | 1.38 (3H, d), 0.92 (3H, t). [M + H]+ 436 | |
| 246 | 1H NMR (400 MHz, Me-d3-OD): 8.54 (1H, d), 7.97-7.87 (1H, m), 7.70-7.57 (2H, m), 7.52 (1H, dd), | As Example 112 steps 1 & 3 using 2- |
| | 4.68 (1H, dd), 3.56-3.41 (1H, m), 2.73-2.56 (2H, m), 2.32-2.00 (2H, m), 1.40 (3H, d), 0.93 (3H, t). | cyano-5-chloro-pyridine in step 1. |
| | [M + H]+ 391 | Followed by Example 88. Separation of |
| | | diastereomers by preparative hplc. |
| 247 | 1H NMR (400 MHz, Me-d3-OD): 8.53 (1H, d), 7.91 (1H, d), 7.71-7.57 (2H, m), 7.50 (1H, dd), | As Example 246 |
| | 4.69 (1H, dd), 3.72-3.59 (1H, m), 2.73-2.54 (2H, m), 2.33-2.18 (1H, m), 2.16-2.00 (1H, m), 1.39 (3H, d), | |
| | 0.94 (3H, t). [M + H]+ 391 | |
| 248 | 1H NMR (400 MHz, Me-d3-OD): 8.42 (1H, d), 8.13 (1H, d), 7.65 (1H, dd), 7.59 (1H, dd), 7.43 (1H, | Example 248 |
| | dd), 4.69 (1H, dd), 3.72-3.61 (1H, m), 2.73-2.53 (2H, m), 2.33-2.19 (1H, m), 2.15-2.00 (1H, m), | |
| | 1.39 (3H, d), 0.94 (3H, t). [M + H]+ 409 | |
| 249 | 1H NMR (400 MHz, Me-d3-OD): 8.44 (1H, d), 8.14 (1H, d), 7.68-7.53 (2H, m), 7.47 (1H, dd), | As Example 248 |
| | 4.68 (1H, dd), 3.50-3.41 (1H, m), 2.72-2.55 (2H, m), 2.28-2.01 (2H, m), 1.40 (3H, d), 1.02-0.88 (3H, m). | |
| | [M + H]+ 409 | |
| 250 | 1H NMR (400 MHz, Me-d3-OD): 7.63-7.49 (2H, m), 7.41-7.30 (2H, m), 7.11 (1H, t), 6.91 (2H, d), | As Example 28 using (S)-1-benzyl-3-(Boc- |
| | 4.66 (1H, dd), 4.46-4.34 (1H, m), 3.56-3.41 (3H, m), 3.41-3.35 (1H, m), 3.28 (1H, dd), | amino)-pyrrolidine in step 2 followed by |
| | 2.75-2.59 (2H, m), 2.37-1.97 (4H, m), 1.38 (3H, d), 0.92 (3H, t). [M + H]+ 434 | Example 223, Step 2. |
| 251 | 1H NMR (400 MHz, Me-d3-OD): 7.63-7.52 (2H, m), 7.41-7.29 (2H, m), 7.11 (1H, t), 6.90 (2H, d), | As Example 28 using (S)-1-N-Boc- |
| | 4.66 (1H, dd), 4.21-4.07 (1H, m), 3.54-3.41 (1H, m), 3.05 (1H, dd), 2.95 (1H, dd), 2.78 (1H, dd), | propane-1,2-diamine in step 2 followed by |
| | 2.63 (1H, dd), 2.30-2.07 (2H, m), 1.39 (3H, d), 1.23 (3H, d), 0.91 (3H, t). | Example 223, Step 2. |
| | [M + H]+ 422 | |
| 252 | 1H NMR (400 MHz, Me-d3-OD): 7.58 (2H, d), 7.41-7.30 (2H, m), 7.11 (1H, t), 6.90 (2H, d), | As Example 28 using tert-Butyl-2-amino- |
| | 4.65 (1H, dd), 3.53-3.40 (1H, m), 3.27 (2H, dd), 2.78-2.59 (2H, m), 2.30-2.07 (2H, m), 1.47-1.35 (9H, | 2-methylpropylcarbamate in step 2 |
| | m), 0.91 (3H, t). [M + H]+ 436 | followed by Example 223, Step 2. |
| 253 | 1H NMR (400 MHz, Me-d3-OD): 7.94 (1H, d), 7.62 (1H, d), 7.60-7.51 (1H, m), 6.93 (1H, d), | As Example 248 using 3-methoxy-4-nitro- |
| | 6.48 (1H, dd), 4.67 (1H, dd), 3.95 (3H, s), 3.51-3.40 (1H, m), 2.70-2.53 (2H, m), 2.25-2.03 (2H, m), | fluorobenzene in step 1 |
| | 1.39 (3H, d), 0.93 (3H, t). [M + H]⁺ 440 | |
| 254 | 1H NMR (400 MHz, Me-d3-OD): 7.61 (1H, dd), 7.56 (1H, dd), 7.33 (1H, d), 6.95 (1H, d), 6.46 (1H, | As Example 253, followed by Example |
| | dd), 4.68 (1H, dd), 3.99 (3H, s), 3.71-3.60 (1H, m), 2.71-2.57 (2H, m), 2.31-2.20 (1H, m), | 238 step 3. Separation of diastereomers |
| | 2.12-1.99 (1H, m), 1.39 (3H, d), 0.92 (3H, t). [M + H]⁺ 410 | by column chromatography |
| 255 | 1H NMR (400 MHz, Me-d3-OD): 7.52 (1H, dd), 7.44 (1H, dd), 6.71 (1H, d), 6.61 (1H, d), 6.20 (1H, | As Example 254 |
| | dd), 4.66-4.51 (2H, m), 3.83 (3H, s), 2.63-2.54 (2H, m), 2.22-2.10 (1H, m), 2.10-1.98 (1H, m), | |
| | 1.34 (3H, d), 0.89 (3H, t). [M + H]⁺ 410 | |
| 256 | 1H NMR (400 MHz, Me-d3-OD): 7.59 (1H, dd), 7.54 (1H, dd), 7.41-7.30 (2H, m), 7.12 (1H, t), | As Example 277, step 1 and step 2 then |
| | 6.90 (2H, d), 4.77 (1H, dd), 3.95-3.82 (2H, m), 3.49-3.39 (1H, m), 3.39-3.20 (2H, m), 2.63 (1H, dd), | as Example 223 using ammonium |
| | 2.58 (1H, dd), 2.23-2.09 (1H, m), 2.06-1.93 (1H, m), 1.72-1.60 (1H, m), 1.54-1.43 (1H, m), | chloride |
| | 1.43-1.28 (6H, m). [M + H]+ 435.2 | |
| 257 | 1H NMR (400 MHz, Me-d3-OD): 7.60 (1H, dd), 7.53 (1H, dd), 7.41-7.30 (2H, m), 7.12 (1H, t), | As Example 256 |
| | 6.90 (2H, d), 4.83-4.76 (1H, m), 3.96-3.80 (2H, m), 3.69-3.55 (1H, m), 3.40-3.23 (2H, m), 2.65 (1H, dd), | |
| | 2.56 (1H, dd), 2.12-2.04 (2H, m), 1.73-1.61 (1H, m), 1.57-1.47 (1H, m), 1.42-1.29 (6H, m). [M + H]+ | |
| | 435.2 | |
| 258 | 1H NMR (400 MHz, Me-d3-OD): 7.59 (1H, dd), 7.54 (1H, dd), 7.41-7.30 (2H, m), 7.12 (1H, t), | As Example 277, step 1 and step 2 using |
| | 6.90 (2H, d), 4.77 (1H, dd), 3.95-3.82 (2H, m), 3.53-3.37 (1H, m), 3.37-3.21 (2H, m), 2.63 (1H, dd), | Example 276B then as Example 223 |
| | 2.59 (1H, dd), 2.23-2.11 (1H, m), 2.10-1.94 (1H, m), 1.72-1.60 (1H, m), 1.54-1.42 (1H, m), | using ammonium chloride |
| | 1.42-1.28 (6H, m). [M + H]+ 435.2 | |
| 259 | 1H NMR (400 MHz, Me-d3-OD): 7.56 (1H, dd), 7.45 (1H, dd), 6.97 (1H, dd), 6.91-6.81 (2H, m), | As Example 248 using 3-trifluoromethyl-4- |
| | 4.63 (1H, d), 3.63 (1H, s), 2.65 (1H, dd), 2.55 (1H, dd), 2.28-2.17 (1H, m), 2.08-1.97 (1H, m), | nitro-fluorobenzene in step 1. Followed by |
| | 1.35 (3H, d), 0.90 (3H, t). [M + H]⁺ 448 | Example 238, step 3. Separation of |
| | | diastereomers by column chromatography |
| | | after reductive amination step. |
| 260 | 1H NMR (400 MHz, Me-d3-OD): 7.56 (1H, dd), 7.47 (1H, dd), 6.98 (1H, dd), 6.92-6.81 (2H, m), | As Example 259 |
| | 4.65 (1H, dd), 3.47-3.37 (1H, m), 2.70-2.52 (2H, m), 2.26-2.00 (2H, m), 1.37 (3H, d), 0.89 (3H, t). | |
| | [M + H]⁺ 448 | |
| 261 | 1H NMR (400 MHz, DMSO-d6): 8.62 (2H, br s), 7.84 (1H, d), 7.70 (1H, d), 7.37 (2H, t), 7.10 (1H, | Example 261 |
| | t), 6.82 (2H, d), 4.58 (1H, s), 2.09-1.97 (1H, m), 1.94-1.82 (1H, m), 0.84 (3H, t). [M + H]+ 296 | |
| 262 | 1H NMR (400 MHz, DMSO-d6): 8.71 (2H, br s), 7.83 (1H, d), 7.74 (1H, d), 7.36 (2H, t), 7.10 (1H, | As Example 261, using minor |
| | t), 6.82 (2H, d), 4.57 (1H, s), 2.11-1.98 (1H, m), 1.95-1.81 (1H, m), 0.84 (3H, t). [M + H]+ 296 | diastereoisomer in final step |
| 263 | 1H NMR (400 MHz, Me-d3-OD): 7.63-7.48 (2H, m), 7.41-7.30 (2H, m), 7.12 (1H, t), 6.91 (2H, d), | As Example 28 using (R)-1-benzyl-3- |
| | 4.66 (1H, dd), 4.48-4.36 (1H, m), 3.56-3.43 (3H, m), 3.43-3.35 (1H, m), 3.27-3.12 (1H, m), | (Boc-amino)pyrrolidine in step 2 followed |
| | 2.67 (2H, d), 2.39-2.28 (1H, m), 2.28-2.16 (1H, m), 2.16-1.97 (3H, m), 1.39 (3H, d), 0.91 (3H, t). [M + H]+ | by Example 223, Step 2. |
| | 434 | |
| 264 | 1H NMR (400 MHz, Me-d3-OD): 7.62-7.50 (2H, m), 7.34 (2H, t), 7.11 (1H, t), 6.91 (2H, dd), | As Example 28 using N-Boc-4- |
| | 4.72-4.54 (2H, m), 4.00 (1H, d), 3.53-3.37 (2H, m), 3.25-3.11 (1H, m), 2.92-2.81 (1H, m), 2.81-2.67 (2H, | aminopiperidine in step 2 followed by |
| | m), 2.28-1.99 (4H, m), 1.69-1.44 (2H, m), 1.41 (3H, dd), 0.92 (3H, t). [M + H]+ 448 | Example 223, Step 2. |
| 265 | 1H NMR (400 MHz, Me-d3-OD): 7.62-7.52 (3H, m), 7.52-7.45 (3H, m), 7.42 (1H, dd), | As Example 28 using 1-Benzyl-1,3- |
| | 7.38-7.27 (2H, m), 7.10 (1H, t), 6.88 (2H, d), 4.66 (1H, dd), 4.25 (2H, dd), 3.65-3.42 (3H, m), 3.20 (2H, t), | propanediamine in step 2 followed by |
| | 2.80-2.60 (2H, m), 2.28-2.08 (2H, m), 1.38 (3H, d), 0.91 (3H, t). [M + H]+ 498 | Example 223, Step 2. |
| 266A | — | Example 266, Step1 |
| 266B | — | Example 266, Step 1. |
| 266C | 1H NMR (400 MHz, Me-d3-OD): 7.66-7.52 (2H, m), 7.47 (1H, d), 7.24 (1H, d), 7.03 (1H, dd), | Example 266 |
| | 4.68 (1H, dd), 3.53-3.41 (1H, m), 2.70-2.59 (2H, m), 2.27-2.16 (1H, m), 2.16-2.04 (1H, m), 1.39 (3H, d), | |
| | 0.92 (3H, t). [M + H]+ 414 | |
| 267 | 1H NMR (400 MHz, Me-d3-OD): 7.67-7.60 (1H, m), 7.57 (1H, dd), 7.47 (1H, d), 7.24 (1H, d), | As Example 266 |
| | 7.03 (1H, dd), 4.85 (23H, s), 4.68 (1H, dd), 3.73-3.62 (1H, m), 3.39-3.28 (22H, m), 2.72-2.56 (2H, m), | |
| | 2.32-2.20 (1H, m), 2.13-1.99 (1H, m), 1.39 (3H, d), 0.93 (3H, t). [M + H]+ 414 | |
| 268 | 1H NMR (400 MHz, Me-d3-OD): 7.58 (1H, dd), 7.48 (1H, dd), 7.41-7.29 (2H, m), 7.11 (1H, t), | As Example 28 using (R)-1-Amino-2- |
| | 6.90 (2H, d), 4.66 (1H, dd), 3.90-3.78 (1H, m), 3.71-3.59 (1H, m), 3.26 (1H, dd), 3.15 (1H, dd), | propanol in step 2. |
| | 2.67 (1H, dd), 2.58 (1H, dd), 2.31-2.16 (1H, m), 2.14-1.98 (1H, m), 1.37 (3H, d), 1.16 (3H, d), 0.93 (3H, | |
| | t). [M + H]+ 423 | |
| 269 | 1H NMR (400 MHz, Me-d3-OD): 7.58 (1H, dd), 7.48 (1H, dd), 7.41-7.29 (2H, m), 7.11 (1H, t), | As Example 28 using 2-Amino-2-methyl- |
| | 6.91 (2H, d), 4.65 (1H, dd), 3.70-3.52 (3H, m), 2.62 (1H, dd), 2.58-2.44 (1H, m), 2.31-2.16 (1H, m), | 1-propanol in step 2. |
| | 2.10-2.02 (1H, m), 1.36 (3H, d), 1.29 (6H, s), 0.93 (3H, t). [M + H]+ 437 | |
| 270 | 1H NMR (400 MHz, Me-d3-OD): 7.58 (1H, dd), 7.49 (1H, d), 7.45 (1H, d), 7.06 (1H, d), 6.93 (1H, | As Example 132, step 1 using Key |
| | dd), 4.53 (1H, dd), 3.68 (2H, s), 2.16-2.06 (1H, m), 2.05-1.97 (1H, m), 1.45 (3H, s), 1.44 (3H, s), | Intermediate 3 and 1-(tert- |
| | 0.97 (3H, t). [M + H]+ 349 | butoxycarbonyl)-3,3-dimethylindolin-5-yl- |
| | | 5-boronic acid followed by Key |
| | | Intermediate 1, Step 6. |
| 271 | 1H NMR (400 MHz, DMSO-d6): 9.57 (2H, br s), 8.08 (3H, br s), 7.70 (1H, t), 7.62 (1H, dd), | Example 271 |
| | 7.43-7.32 (2H, m), 7.12 (1H, t), 6.95 (2H, d), 4.22 (2H, s), 3.10 (1H, s), 2.98 (1H, s), 2.20 (2H, d), | |
| | 2.04 (2H, d), 1.53 (2H, q), 1.39 (2H, q). [M + H]+ 349.0 | |
| 272 | 1H NMR (400 MHz, DMSO-d6): 9.44 (2H, s), 8.09 (3H, s), 7.54 (1H, t), 7.41-7.31 (2H, m), | Example 272 |
| | 7.27 (1H, d), 7.08 (1H, t), 6.88 (2H, d), 4.24-4.13 (2H, m), 3.13-2.91 (2H, m), 2.26-2.12 (2H, m), | |
| | 2.18 (3H, s), 2.11-1.97 (2H, m), 1.53 (2H, q), 1.39 (2H, q). [M + H]+ 329.3 | |
| 273B | 1H NMR (400 MHz, DMSO-d6): 10.49 (1H, br s), 8.07 (3H, br s), 7.84-7.73 (1H, m), 7.65 (1H, d), | Example 273 |
| | 7.38 (2H, t), 7.12 (1H, t), 6.95 (2H, d), 4.57-4.46 (1H, m), 4.35-4.23 (1H, m), 3.29-3.06 (3H, m), | |
| | 3.06-2.92 (1H, m), 2.25-1.99 (4H, m), 1.84-1.63 (2H, m), 1.52-1.32 (2H, m), 1.27 (3H, t). [M + H]+ | |
| | 377.0 | |
| 274 | 1H NMR (400 MHz, DMSO-d6): 8.64 (2H, br s), 7.72-7.58 (2H, m), 6.95-6.85 (1H, m), 6.73 (1H, s), | Example 274 |
| | 6.66 (1H, dt), 4.47 (2H, s), 4.45-4.35 (1H, m), 2.09-1.95 (1H, m), 1.93-1.78 (1H, m), 0.82 (3H, t). | |
| | [M + H]+ 328.0 | |
| 275 | 1H NMR (400 MHz, DMSO-d6): 12.14 (2H, s), 8.66 (1H, d), 7.52 (1H, dd), 7.43-7.31 (3H, m), | Example 275 |
| | 7.11 (1H, t), 6.97 (2H, s), 6.90 (2H, d), 4.86 (1H, dd), 1.93-1.78 (2H, m), 0.94 (3H, t). [M + H]+ 346.0 | |
| 276A | 1H NMR (400 MHz, Me-d3-OD): 7.55 (1H, dd), 7.46 (1H, dd), 7.41-7.26 (2H, m), 7.11 (1H, t), | Example 276 |
| | 6.89 (2H, d), 4.70 (1H, dd), 3.97-3.81 (2H, m), 3.41-3.23 (2H, m), 2.08-1.86 (2H, m), 1.67 (1H, d), | |
| | 1.56 (1H, d), 1.51-1.24 (3H, m). [M + H]+ 350.0 | |
| 276B | 1H NMR (400 MHz, Me-d3-OD): 7.56 (1H, dd), 7.46 (1H, dd), 7.39-7.29 (2H, m), 7.12 (1H, t), | Example 276 |
| | 6.89 (2H, d), 4.71 (1H, dd), 3.97-3.86 (2H, m), 3.41-3.24 (2H, m), 2.10-1.87 (2H, m), 1.73-1.62 (1H, m), | |
| | 1.62-1.51 (1H, m), 1.51-1.26 (3H, m). [M + H]+ 350.0 | |
| 277 | 1H NMR (400 MHz, Me-d3-OD): 7.69-7.55 (2H, m), 7.41-7.30 (2H, m), 7.12 (1H, t), 6.90 (2H, d), | Example 277 |
| | 4.79 (1H, dd), 3.95-3.82 (2H, m), 3.55-3.41 (3H, m), 3.37-3.20 (2H, m), 3.07 (2H, t), 2.79-2.60 (2H, | |
| | m), 2.27-2.14 (1H, m), 2.09-1.97 (1H, m), 1.66 (1H, d), 1.47 (1H, d), 1.43-1.27 (6H, m). [M + H]+ | |
| | 478.2 | |
| 278 | 1H NMR (400 MHz, Me-d3-OD): 7.66-7.55 (2H, m), 7.41-7.30 (2H, m), 7.12 (1H, t), 6.90 (2H, d), | Example 277 |
| | 4.86-4.78 (1H, m), 3.89 (2H, t), 3.72-3.60 (1H, m), 3.57-3.42 (2H, m), 3.39-3.22 (2H, m), 3.09 (2H, | |
| | t), 2.69 (2H, dd), 2.11 (2H, t), 1.68 (1H, d), 1.49 (1H, d), 1.45-1.29 (6H, m). [M + H]+ 478.2 | |
| 279 | ¹H NMR (400 MHz, DMSO-d6): 8.56 (3H, s), 7.68-7.57 (1H, m), 7.54-7.47 (1H, m), | Example 279 |
| | 7.47-7.39 (2H, d), 7.02 (2H, d), 4.40 (1H, dd), 2.07-1.95 (1H, m), 1.93-1.80 (1H, m), 0.82 (3H, t). [MH]⁺ = | |
| | 298/300 | |
| 280 | 1H NMR (400 MHz, Me-d3-OD): 7.47 (1H, t), 7.40 (2H, t), 7.28 (1H, d), 7.24-7.13 (2H, m), | As Example 5/6 using 3-phenoxy- |
| | 7.13-6.98 (3H, m), 4.25 (2H, s), 3.29-3.11 (2H, m), 2.34 (2H, d), 2.21 (2H, d), 1.59 (4H, septet). [M + H]+ | benzylamine in step 1. Separation of |
| | 297.25 | diastereomers by preparative hplc. |
| 281 | 1H NMR (400 MHz, Me-d3-OD): 7.47 (1H, t), 7.40 (2H, t), 7.32 (1H, d), 7.24 (1H, s), 7.18 (1H, t), | As Example 280 |
| | 7.12-6.97 (3H, m), 4.27 (2H, s), 3.54-3.43 (1H, m), 3.40-3.32 (1H, m), 2.19-1.82 (8H, m). [M + H]+ | |
| | 297.25 | |
| 282 | 1H NMR (400 MHz, Me-d3-OD): 7.59-7.50 (1H, m), 7.40-7.32 (2H, m), 7.32-7.24 (1H, m), | As Example 5/6 using 2,4-difluoro-3- |
| | 7.12 (1H, t), 6.97 (2H, d), 4.37 (2H, s), 3.38-3.27 (1H, m), 3.26-3.16 (1H, m), 2.38 (2H, d), 2.22 (2H, d), | phenoxy-benzylamine hydrochloride |
| | 1.61 (4H, septet). [M + H]+ 333.0 | (Example 110, Step 3) in step 1. |
| | | Separation of diastereomers by |
| | | preparative hplc. |
| 283 | 1H NMR (400 MHz, DMSO-d6): 9.49 (2H, br s), 8.13 (3H, br s), 7.78-7.67 (1H, m), 7.49-7.33 (3H, | As Example 282 |
| | m), 7.14 (1H, t), 7.02 (2H, d), 4.31-4.20 (2H, m), 3.34-3.18 (2H, m), 2.06-1.82 (6H, m), 1.75 (2H, | |
| | d). [M + H]+ 333.0 | |
| 284 | 1H NMR (400 MHz, Me-d3-OD): 7.35 (1H, t), 7.31-7.26 (1H, m), 7.17-7.10 (1H, m), 6.99-6.91 (1H, | As Key Intermediate 6 using (3- |
| | m), 6.91-6.83 (1H, m), 6.69 (1H, dd), 3.86 (2H, s), 2.95 (3H, s), 2.88-2.74 (1H, m), 2.52-2.40 (1H, | methylsulfonylamino-phenyl)-boronic acid |
| | m), 2.08-2.00 (2H, m), 2.00-1.93 (2H, m), 1.37-1.17 (4H, m). [M + H]+ 426.0 | in step 1 then as Example 5/6. Separation |
| | | of diastereomers by preparative hplc. |
| 285 | 1H NMR (400 MHz, Me-d3-OD): 7.43-7.32 (1H, m), 7.28 (1H, t), 7.19-7.07 (1H, m), 6.94 (1H, dd), | As Example 284 |
| | 6.88 (1H, t), 6.69 (1H, dd), 3.85 (2H, s), 3.09-2.98 (1H, m), 2.95 (3H, s), 2.77-2.67 (1H, m), | |
| | 1.78-1.61 (8H, m). [M + H]+ 426.0 | |
| 286 | 1H NMR (400 MHz, Me-d3-OD): 7.48-7.37 (2H, m), 7.33-7.26 (3H, m), 7.22-7.12 (1H, m), | As Key Intermediate 6 using (3- |
| | 6.97-6.90 (2H, m), 6.73 (1H, dd), 6.30 (1H, d), 5.86-5.78 (1H, m), 4.50 (2H, s), 2.96 (3H, s). | methylsulfonylamino-phenyl)-boronic acid |
| | | in step 1 then Example 113 step 2 using |
| | | 4-chloro-2-nitropyridine followed by |
| | | Example 19 step 2 |
| 287 | 1H NMR (400 MHz, DMSO-d6): 7.32 (1H, q), 7.18 (1H, t), 6.62-6.46 (3H, m), 4.60 (2H, s), | As Key Intermediate 5, Step1 using 5- |
| | 3.72 (2H, s), 2.61 (1H, s), 2.27 (1H, d), 2.01 (3H, s), 1.86 (2H, s), 1.76 (2H, s), 1.20-0.94 (4H, m). | fluoro 2-nitrotoluene, Example 5/6 |
| | | Example 19 step 2 followed by separation |
| | | of diastereomers by prep hplc and |
| | | deprotection as Example 5/6 step 2 |
| 288 | 1H NMR (400 MHz, DMSO-d6): 7.40-7.28 (1H, m), 7.24-7.13 (1H, m), 6.62-6.47 (3H, m), | As Example 287 |
| | 4.65-4.55 (2H, m), 3.71 (2H, s), 2.71 (1H, d), 2.01 (3H, s), 1.65-1.31 (8H, m). | |
| 289 | 1H NMR (400 MHz, DMSO-d6): 9.44 (2H, br s), 8.03 (3H, br s), 7.73 (1H, t), 7.61 (1H, d), | As Example 271 using tert-butyl(trans-4- |
| | 7.37 (2H, t), 7.12 (1H, t), 6.94 (2H, d), 4.20 (2H, s), 3.00-2.87 (1H, m), 2.87-2.76 (2H, m), 1.97 (2H, d), | amino-methylcyclohexyl)carbamate |
| | 1.88 (2H, d), 1.79-1.65 (1H, m), 1.30 (2H, q), 1.04 (2H, q). [M + H]+ 363.27 | |
| 290 | 1H NMR (400 MHz, DMSO-d6): 9.54 (2H, br s), 8.10 (3H, br s), 7.82 (1H, dd), 7.59 (1H, t), | As Example 271 using 1-bromomethyl-2- |
| | 7.43-7.32 (2H, m), 7.12 (1H, t), 6.93 (2H, d), 4.36-4.24 (2H, m), 3.22-3.08 (1H, m), 3.06-2.93 (1H, m), | chloro-4-fluoro-3-phenoxy-benzene in |
| | 2.25 (2H, d), 2.06 (2H, d), 1.58 (2H, q), 1.42 (2H, q). [M + H]+ 349.0 | step 1 |
| 291 | 1H NMR (400 MHz, Me-d3-OD): 7.45-7.29 (4H, m), 7.13 (1H, t), 6.96 (2H, d), 6.83 (1H, dd), | As Example 5/6 using Pyrrole-2- |
| | 6.28 (1H, d), 6.15 (1H, t), 4.44 (1H, dd), 4.21 (1H, d), 4.16-4.08 (1H, m), 2.25-2.15 (1H, m), | carboxaldehyde in Step 1. |
| | 2.06-1.96 (1H, m), 0.88 (3H, t). | |
| 292 | 1H NMR (400 MHz, Me-d3-OD): 7.60-7.47 (3H, m), 7.35 (2H, t), 7.31-7.24 (1H, m), 7.11 (1H, t), | As Example 5/6 using imidazole-2- |
| | 6.93 (2H, d), 4.41-4.30 (2H, m), 4.25 (1H, d), 2.21-2.08 (1H, m), 2.05-1.89 (1H, m), 0.92 (3H, t). | carboxaldehyde in Step 1. |
| 293 | 1H NMR (400 MHz, Me-d3-OD): 7.52-7.41 (1H, m), 7.41-7.30 (3H, m), 7.13 (1H, t), 6.95 (2H, d), | As Example 5/6 using cyclopentane- |
| | 4.49 (1H, dd), 3.03 (1H, dd), 2.82 (1H, dd), 2.29-2.12 (2H, m), 2.11-1.97 (1H, m), 1.97-1.83 (2H, | carboxaldehyde in Step 1. |
| | m), 1.76-1.57 (4H, m), 1.32-1.15 (2H, m), 0.89 (3H, t). | |
| 294 | 1H NMR (400 MHz, DMSO-d6): 8.56 (3H, s), 7.62-7.49 (2H, m), 7.45-7.34 (2H, m), 7.19-7.09 (1H, | Example 294 |
| | m), 6.95 (2H, d), 4.83 (1H, br s), 4.44 (1H, dd), 3.51-3.39 (1H, m), 3.31 (1H, m), 2.21-2.08 (1H, m), | |
| | 2.05-1.93 (1H, m). LC/MS [M + NH₂]⁺ = 263 | |
| 295 | 1H NMR (400 MHz, Me-d3-OD): 8.11 (2H, s), 7.43-7.36 (2H, m), 7.36-7.30 (2H, m), 7.18-7.08 (1H, | Made using methods described herein |
| | m), 6.93 (4H, dd), 4.57 (1H, dd), 3.46-3.36 (2H, m), 2.52-2.32 (2H, m). | |
| 296 | 1H NMR (400 MHz, Me-d3-OD): 7.41-7.28 (3H, m), 7.21-7.03 (2H, m), 6.91 (2H, d), 3.96-3.83 (2H, | As Example 5/6 using 1-BOC-3- |
| | m), 3.67 (1H, t), 3.55-3.41 (1H, m), 2.94-2.77 (1H, m), 2.77-2.41 (3H, m), 1.94-1.82 (1H, m), | azetidine-carboxaldehyde in Step 1. |
| | 1.73-1.61 (1H, m), 0.84 (3H, t). | |
| 297 | 1H NMR (400 MHz, DMSO-d6): 9.53 (3H, s), 8.87 (1H, s), 8.72 (1H, dd), 8.22-8.15 (1H, m), | Made using methods described herein |
| | 7.76-7.66 (2H, m), 7.56-7.49 (1H, m), 7.42-7.32 (2H, m), 7.16-7.10 (1H, m), 6.97 (2H, d), 6.06 (1H, s), | |
| | 5.33-5.06 (2H, br). [M + H] +313 | |
| 299 | 1H NMR (400 MHz, Me-d3-OD): 7.44-7.26 (4H, m), 7.07 (1H, t), 6.85 (2H, d), 4.03-3.84 (3H, m), | As for example 79 but using Example 356 |
| | 3.49-3.40 (1H, m), 3.40-3.34 (1H, m), 2.90-2.77 (1H, m), 2.26 (2H, d), 2.00 (1H, d), 1.88 (1H, d), | as the starting material. The diastereo- |
| | 1.43-1.19 (3H, m), 1.06 (3H, d). | isomers were separated by preparative |
| | | LC/MS. |
| 300 | 1H NMR (400 MHz, Me-d3-OD): 7.60 (1H, d), 7.54 (1H, dd), 7.35 (2H, t), 7.12 (1H, t), 6.90 (2H, d), | As Example 277, step 1 and step 2 using |
| | 4.80 (1H, t), 3.96-3.82 (2H, m), 3.70-3.55 (1H, m), 3.40-3.22 (2H, m), 2.66 (1H, dd), 2.57 (1H, dd), | Example 276B then as Example 223 |
| | 2.13-2.03 (2H, m), 1.67 (1H, d), 1.51 (1H, d), 1.42-1.29 (6H, m). [M + H]+ 435.2 | using ammonium chloride |
| 301 | 1H NMR (400 MHz, Me-d3-OD): 7.52-7.41 (1H, m), 7.40-7.28 (3H, m), 7.12 (1H, t), 6.96 (2H, d), | Example 53 using key intermediate 1 and |
| | 4.54 (1H, dd), 3.82-3.74 (1H, m), 3.74-3.68 (1H, m), 2.30-2.17 (1H, m), 2.16-2.01 (1H, m), | 2-bromoacetamide |
| | 1.02-0.85 (3H, m). | |
| 302 | 1H NMR (400 MHz, DMSO-d6): 10.71-10.38 (1H, m), 9.14 (2H, br m), 7.66 (1H, q), 7.47 (1H, t), | As Example 91 steps 1-2 using Key |
| | 7.39 (2H, t), 7.14 (1H, t), 6.99 (2H, d), 4.45 (1H, d), 3.12 (1H, s), 3.02 (1H, br s), 2.44 (2H, s), | Intermediate 1 and step 3 using O- |
| | 2.23-2.10 (1H, m), 2.02-1.88 (1H, m), 0.79 (3H, t). | (tertbutyldimethylsilyl)-hydroxylamine |
| 303 | 1H NMR (400 MHz, Me-d3-OD): 8.69 (1H, s), 7.57 (1H, dd), 7.42 (1H, dd), 7.38-7.29 (2H, m), | As Example 148 |
| | 7.11 (1H, t), 6.88 (2H, d), 4.34 (1H, q), 4.04 (1H, dd), 2.17-2.03 (1H, m), 1.99 (3H, s), 1.94-1.80 (1H, | |
| | m), 1.62 (3H, d), 0.81 (3H, t). | |
| 304 | 1H NMR (400 MHz, Me-d3-OD): 7.57 (1H, dd), 7.49 (1H, dd), 7.34 (2H, t), 7.10 (1H, t), 6.90 (2H, | As Example 28, step 2 using |
| | d), 4.64 (1H, dd), 3.96 (1H, septet), 3.49-3.39 (1H, m), 2.62-2.46 (2H, m), 2.27-2.03 (2H, m), | isopropylamine. |
| | 1.36 (3H, d), 1.13 (6H, dd), 0.92 (3H, t). [M + H]+ 407.0 | |
| 305 | 1H NMR (400 MHz, Me-d3-OD): 7.58 (1H, d), 7.47 (1H, dd), 7.35 (2H, t), 7.11 (1H, t), 6.90 (2H, d), | As Example 304 |
| | 4.65 (1H, dd), 4.00 (1H, septet), 3.63 (1H, dd), 2.59 (1H, dd), 2.50 (1H, dd), 2.30-2.16 (1H, m), | |
| | 2.12-1.97 (1H, m), 1.35 (3H, d), 1.16 (6H, dd), 0.93 (3H, t). [M + H]+ 407.0 | |
| 306 | 1H NMR (400 MHz, Me-d3-OD): 7.65-7.50 (2H, m), 7.41-7.29 (2H, m), 7.11 (1H, t), 6.90 (2H, d), | As Example 28, step 2 using hydrazine |
| | 4.70 (1H, dd), 3.74-3.61 (1H, m), 2.86 (1H, dd), 2.72 (1H, dd), 2.35-2.20 (1H, m), 2.17-2.04 (1H, | dihydrochloride. |
| | m), 1.40 (3H, d), 0.91 (3H, t). [M + H]+ 380.0 | |
| 307 | 1H NMR (400 MHz, Me-d3-OD): 7.59 (1H, dd), 7.49 (1H, dd), 7.41-7.29 (2H, m), 7.11 (1H, t), | As Example 28, step 2 using O- |
| | 6.90 (2H, d), 4.68 (1H, dd), 3.79-3.70 (3H, m), 3.70-3.57 (1H, m), 2.56-2.42 (1H, m), 2.32-2.17 (1H, m), | methylhydroxylamine hydrochloride. |
| | 2.14-1.99 (1H, m), 1.37 (3H, d), 0.92 (3H, t). [M + H]+ 395.0 | |
| 308 | 1H NMR (400 MHz, Me-d3-OD): 7.58 (1H, d), 7.48 (1H, t), 7.34 (2H, t), 7.11 (1H, t), 6.90 (2H, d), | As Example 28, step 2 using glycine |
| | 4.66 (1H, dd), 3.98 (2H, s), 3.74 (3H, s), 3.65 (1H, d), 2.76-2.56 (2H, m), 2.30-2.15 (1H, m), | methyl ester hydrochloride. |
| | 2.12-1.97 (1H, m), 1.38 (3H, d), 0.91 (3H, t). [M + H]+ 437.2 | |
| 309 | 1H NMR (400 MHz, Me-d3-OD): 7.57 (1H, d), 7.52-7.42 (1H, m), 7.42-7.29 (2H, m), 7.19-7.06 (1H, | As example 134 using 3-Cyclopropy1-3- |
| | m), 6.91 (2H, d), 4.80-4.70 (1H, m), 2.91-2.74 (2H, m), 2.74-2.62 (1H, m), 2.31-2.15 (1H, m), | oxo-propionitrile in step 1 |
| | 2.15-1.99 (1H, m), 1.16-1.01 (1H, m), 1.01-0.83 (3H, m), 0.83-0.67 (2H, m), 0.48-0.31 (2H, m). | |
| 310 | 1H NMR (400 MHz, Me-d3-OD): 7.58 (1H, dd), 7.48 (1H, dd), 7.41-7.29 (2H, m), 7.11 (1H, t), | As Example 28, step 2 using 1-amino-2- |
| | 6.90 (2H, d), 4.66 (1H, dd), 3.72-3.59 (1H, m), 3.23 (2H, s), 2.70 (1H, dd), 2.60 (1H, dd), 2.31-2.16 (1H, | methyl-propan-2-ol. |
| | m), 2.13-1.98 (1H, m), 1.37 (3H, d), 1.19 (6H, s), 0.93 (3H, t). [M + H]+ 437.2 | |
| 311 | 1H NMR (400 MHz, Me-d3-OD): 7.56 (1H, dd), 7.49 (1H, dd), 7.38-7.31 (2H, m), 7.11 (1H, t), | |
| | 6.90 (2H, d), 4.65 (1H, dd), 3.53-3.39 (1H, m), 3.26-3.16 (2H, m), 2.74-2.54 (2H, m), 2.28-2.05 (2H, m), | As Example 310 |
| | 1.38 (3H, d), 1.17 (6H, d), 0.92 (3H, t). [M + H]+ 437.2 | |
| 312 | 1H NMR (400 MHz, DMSO-d6): 9.90 (1H, s), 9.55-9.47 (1H, m), 9.31-9.22 (1H, m), 7.79-7.66 (3H, | As Example 88, using Example 151. |
| | m), 7.30 (1H, t), 7.19 (1H, s), 6.95 (1H, dd), 6.82 (1H, t), 6.61 (1H, dd), 4.56 (1H, d), 3.41 (1H, d), | |
| | 3.00 (3H, s), 2.58-2.52 (1H, m), 2.44-2.33 (1H, m), 2.22-2.11 (1H, m), 2.00-1.89 (1H, m), 1.24 (3H, | |
| | d), 0.77 (3H, t). [MH]+ = 458/460 | |
| 313 | 1H NMR (400 MHz, Me-d3-OD): 8.09 (1H, d), 7.45-7.35 (2H, m), 7.33-7.19 (2H, m), 4.06 (1H, dd), | As Example 88, using Example 180 |
| | 2.88-2.77 (1H, m), 2.51 (3H, s), 2.29-2.20 (2H, m), 1.93-1.80 (1H, m), 1.75-1.61 (1H, m), 1.07 (3H, | Separation of diastereomers by prep hplc. |
| | d), 0.85 (3H, t). | |
| 314 | 1H NMR (400 MHz, Me-d3-OD): 7.57 (1H, dd), 7.50 (1H, dd), 7.43 (1H, dd), 7.40-7.31 (3H, m), | Example 314 - first eluting isomer. |
| | 7.13 (1H, t), 6.90 (2H, d), 6.29 (1H, t), 4.28 (1H, dd), 4.21 (1H, q), 2.28-2.16 (1H, m), | |
| | 2.09-1.97 (1H, m), 1.63 (3H, d), 0.86 (3H, t). [M + H]+ 401 | |
| 315 | 1H NMR (400 MHz, Me-d3-OD): 7.64 (1H, dd), 7.52-7.44 (2H, m), 7.41 (1H, dd), 7.37-7.29 (2H, | Example 314 - second eluting isomer. |
| | m), 7.15-7.07 (1H, m), 6.87 (2H, d), 6.44 (1H, t), 4.51-4.40 (2H, m), 2.31-2.20 (1H, m), | |
| | 2.13-2.04 (1H, m), 1.66 (3H, d), 0.84 (3H, t). [M + H]+ 401 | |
| 316 | 1H NMR (400 MHz, Me-d3-OD): 7.64-7.52 (2H, m), 7.41-7.30 (2H, m), 7.11 (1H, t), 6.90 (2H, d), | As Example 223 using N-t- |
| | 4.66 (1H, dd), 3.59-3.41 (3H, m), 3.19-2.98 (4H, m), 2.80-2.61 (2H, m), 2.28-2.08 (2H, m), | butyloxycarbonyl-N-ethyl-ethylenediamine |
| | 1.39 (3H, d), 1.34 (3H, t), 0.91 (3H, t). [M + H]+ 436.2 | hydrochloride |
| 317 | 1H NMR (400 MHz, Me-d3-OD): 8.31 (1H, s), 7.67 (1H, d), 7.60 (1H, dd), 7.52 (1H, dd), 7.33 (1H, | As Example 203 using 4-formyl-3-methyl- |
| | s), 6.91 (1H, d), 6.84 (1H, dd), 4.67 (1H, dd), 3.53-3.40 (1H, m), 2.71-2.55 (2H, m), 2.46 (3H, s), | phenylboronic acid then as Example 88. |
| | 2.26-2.05 (2H, m), 1.39 (3H, d), 0.93 (3H, t). | Separation of diastereomers by prep hplc. |
| 318 | 1H NMR (400 MHz, Me-d3-OD): 8.29 (1H, s), 7.67 (1H, d), 7.61 (1H, dd), 7.52 (1H, dd), 7.32 (1H, | As Example 317 |
| | s), 6.92 (1H, d), 6.83 (1H, dd), 4.68 (1H, dd), 3.72-3.61 (1H, m), 2.66 (1H, dd), 2.59 (1H, dd), | |
| | 2.46 (3H, s), 2.32-2.19 (1H, m), 2.14-2.03 (1H, m), 1.38 (3H, d), 0.94 (3H, t). | |
| 319 | 1H NMR (400 MHz, Me-d3-OD): 7.57 (1H, dd), 7.52 (1H, dd), 7.41-7.29 (2H, m), 7.11 (1H, t), | As Example 223 using trans tert-butyl 3- |
| | 6.90 (2H, d), 4.64 (1H, dd), 4.54-4.41 (1H, m), 3.95-3.83 (1H, m), 3.50-3.40 (1H, m), 2.72-2.58 (2H, m), | amino-cyclobutylcarbamate |
| | 2.58-2.38 (4H, m), 2.29-2.00 (2H, m), 1.37 (3H, d), 0.91 (3H, t). [M + H]+ 434.2 | |
| 320 | 1H NMR (400 MHz, Me-d3-OD): 7.57 (1H, dd), 7.52 (1H, dd), 7.41-7.29 (2H, m), 7.11 (1H, t), | As Example 223 using cis-tert-butyl 3- |
| | 6.90 (2H, d), 4.64 (1H, dd), 4.18-4.04 (1H, m), 3.60-3.40 (2H, m), 2.81-2.67 (2H, m), 2.63 (2H, d), | aminocyclobutyl-carbamate |
| | 2.28-2.05 (4H, m), 1.37 (3H, d), 0.91 (3H, t). [M + H]+ 434.2 | |
| 321 | 1H NMR (400 MHz, Me-d3-OD): 7.63-7.52 (2H, m), 7.41-7.30 (2H, m), 7.11 (1H, t), 6.91 (2H, d), | As Example 223 using trans tert-butyl-(2- |
| | 4.66 (1H, dd), 3.61-3.45 (3H, m), 3.25 (2H, t), 2.85-2.61 (3H, m), 2.28-2.07 (2H, m), 1.39 (3H, d), | amino-ethyl)-cyclopropyl-carbamate |
| | 1.01-0.83 (7H, m). [M + H]+ 448.2 | |
| 322 | 1H NMR (400 MHz, Me-d3-OD): 7.66-7.49 (2H, m), 7.38 (1H, d), 7.07-6.99 (1H, m), 6.98-6.89 (1H, | As Example 88 using Example 270, then |
| | m), 4.67 (1H, dd), 3.69-3.62 (2H, m), 3.49-3.42 (1H, m), 2.73-2.52 (2H, m), 2.26-2.01 (2H, m), | deprotection as Example 5/6 step 2 |
| | 1.43 (6H, s), 1.38 (3H, d), 0.95-0.88 (3H, m). | followed by separation of diastereomers |
| | | by prep hplc. |
| 323 | 1H NMR (400 MHz, Me-d3-OD): 7.67-7.50 (2H, m), 7.45 (1H, d), 7.08 (1H, d), 6.95 (1H, dd), | As Example 322 |
| | 4.68 (1H, dd), 3.67 (2H, s), 3.37 (1H, s), 2.71-2.57 (2H, m), 2.31-2.20 (1H, m), 2.11-1.99 (1H, m), | |
| | 1.45 (6H, d), 1.39 (3H, d), 0.92 (3H, t). | |
| 324 | 1H NMR (400 MHz, DMSO-d6): 9.80 (1H, s), 9.43 (1H, s), 7.94 (1H, d), 7.88 (1H, d), 7.67 (1H, s), | As Example 79 using Example 261 |
| | 7.35 (2H, t), 7.16 (1H, s), 7.09 (1H, t), 6.83 (2H, d), 4.70 (1H, d), 3.38 (1H, s), 2.55 (1H, d), | |
| | 2.46-2.33 (1H, m), 2.25 (1H, d), 2.06-1.94 (1H, m), 1.26 (3H, d), 0.77 (3H, t). [MH]+ = 381/383 | |
| 325 | [MH]+ = 381/383 | As Example 324 |
| 326 | 1H NMR (400 MHz, Me-d3-OD): 7.55-7.46 (1H, m), 7.40-7.23 (3H, m), 7.12 (1H, t), 6.97 (2H, d), | As Example 88 using 2,4-difluoro-3- |
| | 4.40 (2H, s), 3.77-3.67 (1H, m), 2.78-2.61 (2H, m), 1.46 (3H, d). | phenoxy-benzylamine hydrochloride |
| | | (Example 110, Step 3) Separation of |
| | | diastereomers by by prep hplc. |
| 327 | 1H NMR (400 MHz, Me-d3-OD): 7.56-7.45 (1H, m), 7.40-7.23 (3H, m), 7.12 (1H, t), 6.97 (2H, d), | As Example 326 |
| | 4.40 (2H, s), 3.77-3.67 (1H, m), 2.78-2.61 (2H, m), 1.46 (3H, d). | |
| 328 | 1H NMR (400 MHz, Me-d3-OD): 7.57-7.44 (2H, m), 7.40-7.29 (2H, m), 7.11 (1H, t), 6.91 (2H, d), | As Example 107 using (S)-3-amino- |
| | 4.40 (2H, s), 3.77-3.65 (1H, m), 2.79-2.59 (2H, m), 1.46 (3H, d). | butyric acid ethyl ester hydrochloride in |
| | | step1 and ammonium chloride in step 3 |
| 329 | 1H NMR (400 MHz, Me-d3-OD): 7.52 (2H, d), 7.40-7.29 (2H, m), 7.11 (1H, t), 6.91 (2H, d), | As Example 107 using (S)-3-amino- |
| | 4.44-4.37 (2H, m), 3.80-3.71 (1H, m), 3.57-3.45 (2H, m), 3.09 (2H, t), 2.74 (2H, d), 1.46 (3H, d). | butyric acid ethyl ester hydrochloride in |
| | | step1 |
| 330 | ¹H NMR (400 MHz, DMSO-d₆): 8.39 (2H, s), 8.31 (2H, s), 8.19 (1H, d), 7.49-7.33 (4H, m), | As Example 8 using Example 77 |
| | 7.10 (1H, t), 6.86 (2H, d), 5.15-5.03 (1H, m), 3.83 (2H, d), 1.95-1.82 (1H, m), 1.82-1.68 (1H, m), | |
| | 0.90 (3H, t). [M + H]+ 387 | |
| 331 | ¹H NMR (400 MHz, DMSO-d₆): 9.99 (1H, s), 9.64 (1H, s), 8.77 (1H, s), 7.98 (3H, s), 7.84-7.74 (1H, | As Example 9 using (2-amino-propyl)- |
| | m), 7.68 (1H, d), 7.39 (2H, t), 7.13 (1H, t), 6.93 (2H, d), 4.31 (1H, s), 3.81 (1H, d), 3.21-3.08 (2H, | carbamic acid tert-butyl ester |
| | m), 2.80 (2H, s), 2.22 (1H, d), 2.07-1.96 (1H, m), 1.77-1.66 (2H, m), 1.40 (3H, d), 0.70 (3H, t). | |
| | [M + H]+ 407 | |
| 332 | ¹H NMR (400 MHz, DMSO-d₆): 10.07 (1H, s), 9.25 (1H, s), 7.77-7.67 (1H, m), 7.63 (1H, d), | As Example 9 using dimethylamine |
| | 7.38 (2H, t), 7.12 (1H, t), 6.95 (2H, d), 4.45 (1H, s), CH obscured by water at 4.3 ppm, 2.95 (3H, s), | |
| | 2.76 (3H, s), 2.35-2.21 (1H, m), 2.11-1.97 (1H, m), 1.41 (3H, d), 0.67 (3H, t). [M + H]+ 379 | |
| 333 | 1H NMR (400 MHz, Me-d3-OD): 7.71-7.58 (2H, m), 7.51 (1H, dd), 7.09-6.98 (1H, m), 6.68 (1H, | Prepared in a manner analogous to |
| | dd), 4.69 (1H, dd), 3.54-3.40 (1H, m), 2.72-2.60 (2H, m), 2.29-2.18 (1H, m), 2.18-2.06 (1H, m), | example 266 starting from 2,4-difluoro-1- |
| | 1.40 (3H, d), 0.93 (3H, t). [M + H]+ = 398/400 | nitrobenzene |
| 334 | 1H NMR (400 MHz, Me-d3-OD): 7.66-7.52 (2H, m), 7.48-7.37 (1H, m), 7.05 (1H, dd), 6.87 (1H, | Example 333 |
| | ddd), 4.67 (1H, dd), 3.53-3.40 (1H, m), 2.71-2.58 (2H, m), 2.28-2.06 (2H, m), 1.39 (3H, d), | |
| | 0.92 (3H, t). [M + H]+ = 398/400 | |
| 335 | 1H NMR (400 MHz, Me-d3-OD): 7.58-7.46 (2H, m), 7.40-7.30 (2H, m), 7.11 (1H, t), 6.91 (2H, d), | As synthesis of key intermediate 1 using |
| | 3.79-3.59 (1H, m), 3.30-3.20 (1H, m), 2.68-2.54 (2H, m), 1.74 (3H, d), 1.38 (3H, d). | 6-chloro-2-fluoro-3-methyl phenol and |
| | | MeLi in step 5 followed by Example 131, |
| | | step 1 and Example 28, using ammonium |
| | | chloride. |
| 336 | 1H NMR (400 MHz, Me-d3-OD): 7.58-7.46 (2H, m), 7.40-7.30 (2H, m), 7.11 (1H, t), 6.91 (2H, d), | Example 335 |
| | 3.79-3.59 (1H, m), 3.30-3.20 (1H, m), 2.68-2.54 (2H, m), 1.74 (3H, d), 1.38 (3H, d). | |
| 337 | 1H NMR (400 MHz, DMSO-d6): 8.59 (3H, s), 7.69-7.58 (2H, m), 7.43-7.32 (2H, m), 7.17-7.07 (1H, | Example 337 |
| | m), 6.91 (2H, d), 4.58 (1H, dd), 3.43-3.35 (1H, m), 3.15 (4H, s), 2.33-2.20 (1H, m), 2.14-2.01 (1H, | |
| | m). [M + H]+ 310. | |
| 338 | 1H NMR (400 MHz, DMSO-d6): 8.60 (2H, s), 7.68-7.57 (2H, m), 7.43-7.32 (2H, m), 7.17-7.07 (1H, | Example 338 |
| | m), 6.92 (2H, d), 4.79 (1H, s), 4.62 (1H, t), 3.53-3.37 (1H, m), 3.31 (1H, s), 2.23-2.11 (1H, m), | |
| | 2.07-1.94 (1H, m). [M + H]+ = 296/298. | |
| 339 | 1H NMR (400 MHz, Me-d3-OD): 7.62-7.47 (2H, m), 7.35 (2H, t), 7.11 (1H, t), 6.91 (2H, d), | As synthesis of key intermediate 1 using |
| | 3.57-3.41 (4H, m), 3.07 (2H, t), 2.67 (2H, s), 1.77 (3H, d), 1.45-1.25 (4H, m). | 6-chloro-2-fluoro-3-methyl phenol and |
| | | MeLi in step 5 followed by Example 131, |
| | | step 1 and Example 223 |
| 340 | 1H NMR (400 MHz, Me-d3-OD): 7.61-7.51 (2H, m), 7.35 (2H, t), 7.11 (1H, t), 6.91 (2H, d), | As Example 339 |
| | 3.79-3.67 (1H, m), 3.58-3.43 (3H, m), 3.09 (2H, t), 2.75-2.64 (2H, m), 1.77 (3H, d), 1.40 (3H, d). | |
| 341 | 1H NMR (400 MHz, Me-d3-OD): 7.52 (1H, dd), 7.49-7.40 (1H, m), 7.40-7.29 (2H, m), 7.11 (1H, t), | As synthesis of key intermediate 1 using |
| | 6.90 (2H, d), 4.76 (1H, q), 1.69 (3H, d). | 6-chloro-2-fluoro-3-methyl phenol and |
| | | MeLi in step 5 |
| 342 | 1H NMR (400 MHz, Me-d3-OD): 7.52 (1H, dd), 7.43 (1H, dd), 7.38-7.29 (2H, m), 7.11 (1H, t), | As Example 341 |
| | 6.89 (2H, d), 4.75 (1H, q), 1.68 (3H, d). | |
| 343 | 1H NMR (400 MHz, DMSO-d6): 8.57 (3H, s), 7.68-7.60 (2H, m), 7.43-7.32 (2H, m), 7.17-7.07 (1H, | Prepared as for Example 338 4 using (S)- |
| | m), 6.92 (2H, d), 4.79 (1H, s), 4.63 (1H, s), 3.53-3.43 (1H, m), 3.32 (1H, m), 2.22-2.10 (1H, m), | 3-(4-chloro-2-fluoro-3-phenoxy-phenyl)-3- |
| | 2.07-1.94 (1H, m). | ((R)-2-methyl-propane-2-sulfinylamino)- |
| | | propionic acid. |
| 344 | 1H NMR (400 MHz, DMSO-d6): 8.71 (3H, s), 7.70-7.56 (2H, m), 7.38 (2H, t), 7.12 (1H, t), | As for Example 61 using tetrahydropyran- |
| | 6.92 (2H, d), 4.29 (1H, d), 3.93 (1H, d), 3.79 (1H, d), 3.28-3.14 (2H, m), 2.14 (1H, s), 1.83 (1H, d), | 4-carboxaldehyde and (S)-2-methyl-2- |
| | 1.43-1.27 (1H, m), 1.17 (2H, s). | propane sulfonamide in step 2 and tert- |
| | | butyl-(2-chloro-6-fluoro-phenoxy)- |
| | | dimethyl-silane in step 3. |
| 345 | 1H NMR (400 MHz, DMSO-d6): 8.63 (3H, s), 8.09 (1H, d), 7.71 (1H, d), 7.67-7.58 (1H, m), | As for Example 344, using 2-nitro-5- |
| | 7.12 (1H, d), 6.94 (1H, dd), 4.31 (1H, d), 3.94 (1H, d), 3.80 (1H, d), 3.28-3.16 (2H, m), 2.55 (3H, s), | fluorotoluene, K₂CO₃, DMSO in step 5/1. |
| | 2.17-2.06 (1H, m), 1.81 (1H, d), 1.42-1.31 (1H, m), 1.18 (2H, s). | |
| 346 | 1H NMR (400 MHz, Me-d3-OD): 7.53 (1H, dd), 7.45-7.29 (3H, m), 7.11 (1H, t), 6.89 (2H, d), | As Key Intermediate 1 using 6-chloro-2- |
| | 4.49 (1H, d), 2.04-1.90 (1H, m), 1.74-1.47 (2H, m), 1.47-1.30 (1H, m), 1.28-1.11 (1H, m), 1.02 (3H, t), | fluoro-3-methylphenol in step 1 and 3- |
| | 0.85 (3H, t). [M + H]+ 322.0 | pentylmagnesium bromide in step 5. |
| | | Separation of diastereomers at step 5 by |
| | | column chromatography. |
| 347 | 1H NMR (400 MHz, Me-d3-OD): 7.53 (1H, dd), 7.44-7.29 (3H, m), 7.11 (1H, t), 6.89 (2H, d), | As Example 346 |
| | 4.49 (1H, d), 2.04-1.91 (1H, m), 1.73-1.47 (2H, m), 1.46-1.30 (1H, m), 1.28-1.11 (1H, m), 1.02 (3H, t), | |
| | 0.85 (3H, t). [M + H]+ 322.0 | |
| 348 | 1H NMR (400 MHz, DMSO-d6): 9.45 (2H, br d), 7.77 (2H, s), 7.70 (1H, d), 7.36 (2H, t), | As Example 79 using Example 344. |
| | 7.33-7.24 (1H, m), 7.12 (1H, t), 6.92 (2H, d), 4.54 (1H, s), 3.93 (1H, d), 3.80 (1H, d), 3.30-3.16 (2H, m), | |
| | 2.46-2.27 (3H, m), 1.97 (1H, d), 1.43-1.07 (6H, m). | |
| 349 | 1H NMR (400 MHz, DMSO-d6): 9.12-9.06 (1H, m), 7.69 (3H, d), 7.37 (2H, t), 7.30-7.22 (1H, m), | As Example 348 |
| | 7.12 (1H, t), 6.91 (2H, d), 4.56-4.48 (1H, m), 3.93 (1H, d), 3.81 (1H, d), 3.28 (6H, d), | |
| | 2.70-2.60 (1H, m), 2.32 (1H, d), 1.94 (1H, d), 1.42-1.32 (1H, m), 1.17 (4H, s). | |
| 350 | ¹H NMR (400 MHz, Me-d₃-OD): 7.57 (1H, dd), 7.52 (1H, dd), 7.40-7.28 (2H, m), 7.16-7.05 (1H, m), | As Example 79 from Example 338. |
| | 6.90 (2H, d), 4.91 (1H, dd), 3.78-3.68 (1H, m), 3.68-3.57 (1H, m), 3.56-3.42 (1H, m), 2.68 (1H, dd), | Separation of diastereomers by |
| | 2.58 (1H, dd), 2.48-2.35 (1H, m), 2.27-2.14 (1H, m), 1.37 (3H, d). | preparative hplc |
| 351 | 1H NMR (400 MHz, Me-d3-OD): 7.69-7.55 (2H, m), 7.41-7.30 (2H, m), 7.12 (1H, t), 6.90 (2H, d), | As Example 277 using Example 276B in |
| | 4.79 (1H, dd), 3.89 (2H, t), 3.56-3.41 (3H, m), 3.41-3.19 (2H, m), 3.07 (2H, t), 2.79-2.60 (2H, m), | step 1 |
| | 2.27-2.14 (1H, m), 2.14-1.98 (1H, m), 1.73-1.59 (1H, m), 1.55-1.43 (1H, m), 1.42-1.29 (6H, m). | |
| | [M + H]+ 478.2 | |
| 352 | 1H NMR (400 MHz, Me-d3-OD): 7.67-7.56 (2H, m), 7.41-7.30 (2H, m), 7.12 (1H, t), 6.90 (2H, d), | As Example 277 using Example 276B in |
| | 4.88-4.79 (1H, m), 3.89 (2H, t), 3.66 (1H, dd), 3.58-3.42 (2H, m), 3.42-3.22 (2H, m), 3.09 (2H, t), | step 1 |
| | 2.77-2.62 (2H, m), 2.12 (2H, t), 1.74-1.60 (1H, m), 1.57-1.44 (1H, m), 1.41-1.29 (6H, m). [M + H]+ | |
| | 478.2 | |
| 353 | 1H NMR (400 MHz, Me-d3-OD): 7.42-7.25 (4H, m), 7.08 (1H, t), 6.85 (2H, d), 5.09 (1H, t), | As Example 79 using Example 343. |
| | 3.26 (1H, dd), 3.01 (1H, dd), 2.46 (1H, s), 2.15-2.03 (1H, m), 1.94-1.78 (2H, m), 1.10-0.93 (9H, m). | Separation of diastereomers by by prep |
| | | hplc. |
| 354 | 1H NMR (400 MHz, DMSO-d6): 7.53-7.43 (2H, m), 7.43-7.31 (3H, m), 7.15-7.05 (1H, m), | Made using methods described herein |
| | 6.92-6.80 (3H, m), 4.47 (1H, t), 2.47-2.35 (2H, m), 1.10 (2H, s). [M + H]+309 | |
| 355 | 1H NMR (400 MHz, Me-d3-OD): 8.06 (1H, d), 7.86-7.73 (2H, m), 7.60 (1H, dd), 7.50 (1H, t), | As Example 112 using 5-chloro-2- |
| | 4.53 (1H, dd), 2.25 (3H, s), 2.17-2.02 (3H, m), 0.98 (3H, t). | nitropyridine in step 1, then as Example |
| | | 106 |
| 356 | 1H NMR (400 MHz, DMSO-d6): 8.74 (3H, s), 7.69-7.57 (2H, m), 7.37 (2H, t), 7.12 (1H, t), | As for Example 344, but starting from (S)- |
| | 6.92 (2H, d), 4.29 (1H, d), 3.93 (1H, d), 3.79 (1H, d), 3.29-3.14 (2H, m), 2.21-2.08 (1H, m), 1.84 (1H, d), | 2-methyl-propane-2-sulfinic acid amide |
| | 1.43-1.28 (1H, m), 1.23-1.13 (2H, m). | |
| 357 | 1H NMR (400 MHz, Me-d3-OD): 7.54 (1H, dd), 7.45 (1H, dd), 7.39-7.29 (2H, m), 7.16-7.06 (1H, | Example 357 |
| | m), 6.90 (2H, d), 4.80 (1H, dd), 4.74-4.64 (0.5H, m), 4.63-4.47 (1H, m), 4.46-4.36 (0.5H, m), | |
| | 2.60-2.27 (2H, m). {M + H]+ 298. | |
| 358 | 1H NMR (400 MHz, Me-d3-OD): 7.60-7.49 (2H, m), 7.34 (2H, dd), 7.10 (1H, t), 6.90 (2H, d), | As Example 79 using Example 343. |
| | 4.94 (1H, dd), 3.77-3.68 (1H, m), 3.63-3.49 (1H, m), 3.49-3.38 (1H, m), 2.71-2.54 (2H, m), | Separation of diastereomers by by prep |
| | 2.42-2.20 (2H, m), 1.38 (3H, d). | hplc. |
| 359 | 1H NMR (400 MHz, DMSO-d6): 9.83-9.41 (2H, m), 7.78-7.69 (1H, m), 7.67 (1H, dd), | Made using methods described herein |
| | 7.43-7.32 (2H, m), 7.17-7.07 (1H, m), 6.93 (2H, d), 4.53 (1H, dd), 3.42-3.33 (1H, m), 3.12 (3H, s), | |
| | 3.11-3.00 (1H, m), 2.47 (3H, s), 2.46-2.35 (1H, m), 2.20-2.07 (1H, m). | |
| 360 | 1H NMR (270 MHz, CDCl₃): 7.29-7.22 (4H, m), 6.82 (2H, m), 4.12 (1H, t), 1.75-1.62 (2H, m), | Example 360 |
| | 0.89 (3H, t). | |
| 361 | 1H NMR (270 MHz, DMSO-d₆): 9.96 (2H, br s), 8.75 (3H, br s), 7.69-7.61 (2H, m), 7.31 (2H, d), | Example 361 |
| | 7.01 (2H, d), 4.35 (1H, br s), 2.07-1.98 (1H, m), 1.90-1.80 (1H, m), 0.80 (3H, t). | |
| | MS: 278 ([M − NH₂]H⁺) | |
| 362 | ¹H NMR >95%, 2.27 mmol, 74% yield). 1H NMR (270 MHz, DMSO-d₆): 8.83 (3H, s), | Example 362 |
| | 8.29-8.23 (2H, m), 7.78-7.67 (2H, m), 7.22-7.16 (2H, m), 4.38 (1H, q), 2.01-1.81 (2H, m), 0.83 (3H, t). | |
| 363 | 1H NMR (270 MHz, DMSO-d₆): 9.98 (1H, br s), 8.66 (3H, br s), 7.64-7.59 (2H, m), 7.53 (2H, m), | Example 363 |
| | 6.84 (2H, m), 4.37 (1H, br s), 2.06-1.96 (1H, m), 2.00 (3H, s), 1.90-1.77 (1H, m), 0.79 (3H, t). | |
| | MS: 337 (MH⁺) | |
| 364 | 1H NMR (270 MHz, DMSO-d₆): 8.60 (3H, br s), 8.27 (1H, br s), 7.64-7.54 (2H, m), 7.39 (2H, m), | Example364 |
| | 6.78 (2H, m), 4.38 (1H, br s), 2.89 (6H, s), 2.05-1.95 (1H, m), 1.90-1.77 (1H, m), 0.79 (3H, t). | |
| | MS: 366 (MH⁺) | |
| 365 | 1H NMR (270 MHz, DMSO-d₆): 8.63 (3H, s), 7.65-7.58 (2H, m), 6.87 (1H, d), 6.56-6.51 (2H, m), | Example 365 |
| | 4.57 (2H, s), 4.42-4.37 (1H, m), 3.17 (1H, s), 2.05-1.82 (2H, m), 0.80 (3H, t). MS: 334.2 ([M − NH₃]⁺), | |
| | 351.2 (MH⁺) | |
| 366 | 1H NMR (270 MHz, DMSO-d₆): 8.71 (3H, s), 7.67-7.59 (2H, m), 6.95 (1H, d), 6.49-6.37 (2H, m), | Example 366 |
| | 4.43-4.30 (1H, s), 4.25 (2H, bs), 3.44-3.33 (4H, m), 2.07-1.77 (2H, m), 0.79 (3H, t). MS: | |
| | 320.1 ([M − NH₃]⁺), 337.1 (MH⁺) | |
| 367 | 1H NMR (270 MHz, DMSO-d₆): 8.57 (3H, br s), 7.64-7.55 (2H, m), 6.83 (1H, m), 6.38 (2H, m), | Example 367 |
| | 4.38 (1H, dd), 4.22-4.18 (4H, m), 2.05-1.95 (1H, m), 1.90-1.76 (1H, m), 0.78 (3H, t). | |
| | MS: 337 (MH⁺) | |
| 368 | 1H NMR (270 MHz, DMSO-d₆): 8.73 (2H, d), 8.70 (3H, bs), 7.73 (2H, dd), 7.40 (2H, d), 4.39 (1H, | Example 368 |
| | q), 2.10-1.80 (2H, m), 0.81 (3H, t). | |
| | MS: 281.0 (MH⁺). | |
| 369 | 1H NMR (270 MHz, DMSO-d₆): 8.71 (3H, br s), 8.06 (1H, dd), 7.93 (1H, ddd), 7.66-7.56 (2H, m), | Example 369 |
| | 7.25 (1H, d), 7.18 (1H, d), 4.42-4.34 (1H, m), 2.06-1.98 (1H, m), 1.90-1.76 (1H, m), 0.79 (3H, t). | |
| | MS: 281 (MH⁺) | |
| 370 | 1H NMR (270 MHz, DMSO-d₆): 8.81 (3H, bs), 8.02 (1H, d), 7.93 (2H, bs), 7.80-7.70 (2H, m), | Example 370 |
| | 6.77 (1H, dd), 6.41 (1H, dd), 4.41 (1H, bs), 2.10-1.82 (2H, m), 0.81 (3H, t). | |
| | MS: 279.0 ([M − NH₃]⁺), 296.1 (MH⁺). | |
| 371 | 1H NMR (270 MHz, DMSO-d₆): 10.80 (1H, s), 8.70 (3H, bs), 8.25 (1H, d), 7.80-7.65 (2H, m), | Example 371 |
| | 7.62 (1H, d), 6.72 (1H, dd), 4.39 (1H, bq), 2.20-1.70 (2H, m), 2.10 (3H, s), 0.79 (3H, t). | |
| | MS: 321.2 ([M − NH₃]⁺), 338.3 (MH⁺). | |
| 372 | 1H NMR (270 MHz, DMSO-d₆): 8.70 (3H, br s), 7.80-7.76 (2H, m), 7.72 (1H, d), 7.64 (2H, m), | Example 372 |
| | 6.94 (2H, d), 6.66 (1H, d), 4.41 (1H, m), 2.08-1.98 (1H, m), 1.90-1.79 (1H, m), 0.80 (3H, t). | |
| | MS: 345 ([M − NH₂]H⁺) | |
| 373 | 1H NMR (270 MHz, DMSO-d₆): 8.78 (3H, br s), 7.77-7.58 (4H, m), 7.31 (1H, t), 7.15-6.99 (2H, m), | Example 373 |
| | 4.38 (1H, br s), 2.08-1.95 (1H, m), 1.85-1.78 (1H, m), 0.78 (3H, t). | |
| | MS: 341 (MH⁺) | |
| 374 | 1H NMR (270 MHz, DMSO-d₆): 8.63 (3H, br s), 8.33 (1H, br s), 7.69-7.61 (2H, m), 7.32 (1H, t), | Example 374 |
| | 7.15-7.09 (1H, m), 7.04-7.01 (1H, m), 4.39 (1H, dd), 2.72 (3H, d), 2.03-1.95 (1H, m), | |
| | 1.89-1.79 (1H, m), 0.78 (3H, t). | |
| | MS: 355 (MH⁺) | |
| 375 | 1H NMR (270 MHz, DMSO-d₆): 8.73 (3H, br s), 7.70-7.62 (2H, m), 7.32 (1H, t), 7.08-7.02 (1H, m), | Example 375 |
| | 6.97 (1H, dd), 4.38 (1H, br s), 3.61 (4H, br s), 3.51 (2 H, br m), 3.23 (2 H, br m), 2.07-1.94 (1H, m), | |
| | 1.92-1.75 (1H, m), 0.79 (3H, t). | |
| | MS: 411 (MH⁺) | |
| 376 | 1H NMR (270 MHz, DMSO-d₆): 8.72 (3H, br s), 7.69-7.62 (2H, m), 7.31 (1H, t), 7.07-7.01 (1H, m), | Example 376 |
| | 6.90 (1H, dd), 4.38 (1H, dd), 2.95 (3H, s), 2.81 (3H, s), 2.07-1.98 (1H, m), 1.90-1.78 (1H, m), | |
| | 0.78 (3H, t). | |
| | MS: 369 (MH⁺) | |
| 377 | 1H NMR (270 MHz, DMSO-d₆): 8.82 (1H, d), 8.78 (1H, s), 8.60 (3H, bs), 7.65 (2H, dd), 7.46 (1H, | Example 377 |
| | d), 4.40 (1H, q), 2.05-1.75 (2H, m), 0.80 (3H, t). | |
| | MS: 264.9 ([M − NH₃]⁺). | |
| 378 | 1H NMR (270 MHz, DMSO-d₆): 8.64 (3H, bs), 8.07 (1H, s), 7.60 (2H, dd), 7.40-7.10 (2H, bs), | Example 378 |
| | 4.40 (1H, q), 2.10-1.40 (2H, m), 0.81 (3H, t). | |
| | MS: 280.0 ([M − NH₃]⁺), 297.0 (MH⁺). | |
| 379 | 1H NMR (270 MHz, DMSO-d₆): 9.08 (1H, s), 8.86 (3H, bs), 7.88 (1H, dd), 7.80-7.60 (3H, m), | Example 379 |
| | 4.38 (1H, q), 2.15-1.70 (2H, m), 0.79 (3H, t). | |
| | MS: 264.9 ([M − NH₃]⁺), 282.0 (MH⁺). | |
| 380 | 1H NMR (270 MHz, DMSO-d₆): 8.85 (3H, s), 8.80 (1H, s), 8.48 (1H, d), 8.21-8.15 (1H, m), | Example 380 |
| | 7.77-7.61 (2H, m), 4.39-4.38 (1H, m), 2.10-1.74 (2H, m), 0.79 (3H, t). MS: 265.0 ([M − NH₃]⁺) | |
| 381 | 1H NMR (270 MHz, DMSO-d₆): 8.62 (3H, bs), 8.00 (1H, d), 7.57 (2H, dd), 7.36 (1H, d), 4.37 (1H, | Example 381 |
| | q), 2.10-1.70 (2H, m), 0.78 (3H, t). | |
| | MS: 280.1 ([M − NH₃]⁺), 297.1 (MH⁺). | |
| 382 | 1H NMR (270 MHz, DMSO-d₆): 8.79 (3H, s), 8.67 (2H, d), 7.72-7.59 (2H, m), 7.37 (1H, t), | Example 382 |
| | 4.38 (1H, q), 2.09-1.78 (2H, m), 0.80 (3H, t). MS: 264.8 ([M − NH₃]⁺), 281.8 (MH⁺) | |
| 383 | 1H NMR (270 MHz, DMSO-d₆): 8.79 (3H, bs), 8.11 (2H, dd), 7.70-7.50 (2H, m), 5.82 (2H, bs + H₂O), | Example 383 |
| | 4.36 (1H, q), 2.10-1.70 (2H, m), 0.78 (3H, t). | |
| | MS: 280.0 ([M − NH₃]⁺), 297.0 (MH⁺). | |
| 384 | 1H NMR (270 MHz, DMSO-d₆): 8.81 (3H, bs), 8.02 (1H, d), 7.83-7.63 (3H, m), 7.46-7.35 (2H, m), | Example 384 |
| | 4.44 (1H, q), 2.12-1.76 (2H, m), 0.81 (3H, t). MS: 319.7 ([M − NH₃]⁺), 336.7 (MH⁺) | |
| 385 | 1H NMR (270 MHz, DMSO-d₆): 8.89 (3H, bs), 8.07 (1H, d), 7.86-7.69 (2H, m), 7.61 (1H, d), | Example 385 |
| | 7.30 (1H, dd), 4.43 (1H, bs), 2.09-1.82 (2H, m), 0.80 (3H, t). MS: 335.0 ([M − NH₃]⁺), 352.0 (MH⁺) | |
| 386 | 1H NMR (270 MHz, DMSO-d₆): 9.24 (1H, s), 8.91 (3H, s), 8.62 (1H, dd), 7.90-7.72 (2H, m), | Example 386 |
| | 4.46-4.23 (1H, m), 2.12-1.83 (2H, m), 0.81 (3H, t). MS: 321.1 ([M − NH₃]⁺), 338.2 (MH⁺). | |
| 387 | 1H NMR (270 MHz, DMSO-d₆): 8.63 (3H, bs), 7.73-7.67 (2H, m), 4.43 (1H, q), 2.62 (3H, s), | Example 387 |
| | 2.02-1.76 (2H, m), 0.78 (3H, t). MS: 285.0 ([M − NH₃]⁺), 302.0 (MH⁺) | |
| 388 | 1H NMR (270 MHz, CDCl₃): 9.00 (3H, bs), 7.73-7.64 (1H, m), 7.36-7.28 (1H, m), 4.54 (1H, bs), | Example 388 |
| | 2.51 (3H, s) 2.26-1.91 (2H, m), 0.89 (3H, t). MS: 269.1 ([M − NH₃]⁺), 286.1 (MH⁺) | |
| 389 | 1H NMR (270 MHz, DMSO-d₆): 8.69 (3H, bs), 7.74 (1H, d), 7.64 (2H, dd), 7.51 (1H, m), 6.90 (1H, | Example 389 |
| | d), 4.40 (1H, q), 3.11 (6H, s), 2.10-1.50 (2H, m), 0.81 (3H, t). MS: 323.8 (MH⁺). | |
| 390 | 1H NMR (270 MHz, CDCl₃•MeOD-d₄): 7.76-7.73 (2H, m), 7.25-7.16 (2H, m), 6.86 (2H, d), | Example 390 |
| | 4.04 (1H, t), 1.74-1.63 (2H, m), 0.84 (3H, t). MS: 323 (MH⁺) | |
| 391 | 1H NMR (270 MHz, DMSO-d₆): 9.00-8.45 (3H, bs), 8.91 (1H, s), 8.74 (1H, s), 7.68 (2H, m), | Example 391 |
| | 4.42 (1H, q), 2.10-1.75 (2H, m), 0.80 (3H, t). MS: 309.0 ([M − NH₃]⁺), 326.0 (MH⁺). | |
| 392 | 1H NMR (270 MHz, DMSO-d₆): 8.90-8.70 (3H, bs), 8.85 (1H, s), 8.70 (1H, s), 8.20 (1H, bs), | Example 392 |
| | 7.83 (1H, bs), 7.77-7.60 (2H, m), 4.40 (1H, bq), 2.15-1.65 (2H, m), 0.79 (3H, t). | |
| | MS: 308.0 ([M − NH₃]⁺), 325.0 (MH⁺). | |
| 397 | 1H NMR (270 MHz, DMSO-d₆): 8.92 (3H, bs), 7.95-7.55 (4H, m), 7.05 (1H, d), 3.76 (1H, m), | Example 397 |
| | 1.42 (1H, m), 0.75-0.60 (2H, m), 0.58-0.43 (1H, m), 0.35-0.23 (1H, m). MS: 291.0 ([M − NH₃]⁺), | |
| | 308.0 (MH⁺). | |
| 398 | 1H NMR (270 MHz, DMSO-d₆): 8.81 (3H, bs), 8.41 (1H, d), 8.03 (1H, d), 7.97 (1H, bs), | Example 398 |
| | 7.85-7.65 (2H, m), 7.62 (1H, bs), 7.45 (1H, dd), 3.82 (1H, m), 1.50-1.33 (1H, m), 0.80-0.45 (3H, m), | |
| | 0.36-0.25 (1H, m). MS: 319.0 ([M − NH₃]⁺), 336.1 (MH⁺). | |
| 399 | 1H NMR (270 MHz, MeOD-d₄): 7.60-7.45 (2H, m), 7.30 (2H, d), 6.90 (2H, d), 4.63 (1H, dd), | From Example 360 using General |
| | 3.67-3.52 (1H, obs sextet), 2.70-2.50 (2H, 2 × dd), 2.30-1.92 (2H, m), 1.35 (3H, d), 0.89 (3H, t). 99.4% by | Methods 1, 2 and 3. |
| | LCMS, 399.1 (MH⁺). 98.8% d.e., 1H NMR >95%. | Purification on silica (6 g 1:1 mix |
| | | normal/TLC). Eluent: 5% |
| | | MeOH/EtOAc + 0.1% NH₃. |
| | | 2M HCl/EtOAc/Et₂O gave 27 mg (20.7%). |
| 400 | 1H NMR (270 MHz, MeOD-d₄): 7.60-7.42 (2h, m), 7.33 (2H, d), 6.90 (2H, d), 4.61 (1H, dd), | From Example 360 using General |
| | 3.40 (1H, obs sextet), 2.68-2.47 (2H, 2 × dd), 2.21-1.95 (2H, m), 1.34 (3H, d), 0.88 (3H, t). | Methods 1, 2 and 3. |
| | 100% by LCMS, 399.1 (MH⁺). 89.0% d.e., 1H NMR >95%. | Purification on silica (6 g 1:1 mix |
| | | normal/TLC). Eluent: 5% |
| | | MeOH/EtOAc + 0.1% NH₃. |
| | | 2M HCl/EtOAc/Et₂O gave 12 mg (9.2%). |
| 401 | Completion check: LC | From Example 362 using General |
| | Isolated purity: 96.9% by HPLC. | Methods 1, 2 and 3. |
| | | Purification on silica (22 g). Eluent: 10% |
| | | MeOH/EtOAc + 0.2% NH_{3.} Gave 30 mg |
| | | (4% yield). |
| 402 | Completion check: LC | From Example 362 using General |
| | Isolated purity: 97.0% by HPLC. | Methods 1, 2 and 3. |
| | | Purification on silica (22 g). Eluent: 10% |
| | | MeOH/EtOAc + 0.2% NH_{3.} Gave 40 mg |
| | | (6% yield). |
| 403 | 1H NMR (270 MHz, MeOD-d₄): 7.53-7.43 (2H, m), 7.34-7.28 (2H, m), 7.03-697 (2H, m), 4.57 (1H, | From Example 401 using General Method 4 |
| | q), 3.54 (1H, obs sextet), 2.61-2.49 (2H, m), 2.24-1.87 (2H, m), 1.29 (3H, d), 0.82 (3H, t). | Purification on silica (3 g). Eluent: |
| | 99.4% by LCMS, 380.1 (MH⁺), 99.7% d.e., 1H NMR >95%. | 10% MeOH/EtOAc + 0.2% NH₃. 2M HCl/EtOAc/Et₂O gave 20.2 mg (69% |
| | | yield). |
| 404 | 1H NMR (270 MHz, MeOD-d₄): 7.35-7.46 (2H, m), 7.33 (2H, d), 7.00 (2H, d), 4.57 (1H, q), | From Example 402 using General Method 4 |
| | 3.32 (1H, obs sextet), 2.56 (2H, d), 2.16-1.90 (2H, m), 1.28 (3H, d), 0.80 (3H, t). 98.0% by LCMS, | Purification on silica (3 g). Eluent: |
| | 380.1 (MH⁺), 97.6% d.e., 1H NMR >95%. | 10% MeOH/EtOAc + 0.2% NH₃. 2M |
| | | HCl/EtOAc/Et₂O gave 14.2 mg (33% |
| | | yield). |
| 405 | 1H NMR (270 MHz, MeOD-d₄): 7.45 (2H, d), 7.38-7.26 (2H, m), 6.78 (2H, d), 4.03 (1H, dd), | From Example 363 using General |
| | 2.82 (1H, m), 2.22 (2H, d), 2.09 (3H, s), 1.90-1.75 (1H, m), 1.74-1.55 (1H, m), 1.04 (3H, d), 0.81 (3H, t). | Methods 1, 2 and 3. |
| | 95.8% by LCMS, 422.3 (MH⁺). 99.0% d.e., 1H NMR >95%. | Purification on silica (1 g 1:1 mix |
| | | normal/TLC). Eluent: 10% |
| | | MeOH/EtOAc + 1% NH₃ gave 11 mg |
| | | (18.4%). |
| 406 | 1H NMR (270 MHz, MeOD-d₄): 7.55 (1H, dd), 7.43 (1H, dd), 7.32-7.26 (2H, m), 6.84-6.78 (2H, d), | From Example 364 using General |
| | 4.62 (1H, dd), 3.64-3.57 (1H, m), 3.00 (6H, s), 2.67-2.48 (2H, 2 × dd), 2.24-2.16 (1H, m), | Methods 1, 2 and 3. |
| | 2.05-1.98 (1H, m), 1.33 (3H, d), 0.89 (3H, t). 94.5% by LCMS, 451.2 (MH⁺). 99.8% d.e, 1H NMR >95%. | Purification on silica (10 g 1:1 mix |
| | | normal/TLC). Eluent: EtOAc/MeOH/2% aq |
| | | NH₃ in MeOH 90:10:2. 2M HCl/EtOAc/Et₂O |
| | | gave 18 mg (15%). |
| 407 | 1H NMR (270 MHz, MeOD-d₄): 7.54 (1H, dd), 7.45 (1H, dd), 7.33-7.27 (2H, m), 6.83-6.77 (2H, d), | From Example 364 using General |
| | 4.62 (1H, dd), 3.44-3.37 (1H, m), 3.00 (6H, s), 2.64-2.53 (2H, 2 × dd), 2.19-2.07 (2H, m), 1.35 (3H, | Methods 1, 2 and 3. |
| | d), 0.88 (3H, t). 95.0% by LCMS, 451.2 (MH⁺). 99.2% d.e., 1H NMR >95%. | Purification on silica (10 g 1:1 mix |
| | | normal/TLC). Eluent: EtOAc/MeOH/2% aq |
| | | NH₃ in MeOH 90:10:2. 2M HCl/EtOAc/Et₂O gave 27 mg (22%). |
| 408 | 1H NMR (270 MHz, MeOD-d₄): 7.60-7.51 (2H, m), 7.34 (1H, d), 6.69 (1H, dd), 6.57 (1H, d), | From Example 366 using General |
| | 4.65 (1H, q), 4.47-4.44 (2H, m), 3.73-3.52 (3H, m), 2.70-2.53 (2H, m), 2.32-1.94 (2H, m), 1.36 (3H, d), | Methods 1, 2 and 3. |
| | 0.89 (3H, t). MS: 422.2 (MH⁺), 99.4% d.e., 94.3% by LCMS, 1H NMR >95%. | Purification on silica (20 g 2:1 mix |
| | | normal/TLC). Eluent: 5% MeOH/EtOAc + 0.1% NH₃. |
| | | 2.1M HCl/EtOAc/Et₂O gave 22 mg (12% |
| | | yield). |
| 409 | 1H NMR (270 MHz, MeOD-d₄): 7.59-7.50 (2H, m), 7.28 (1H, d), 6.66 (1H, dd), 6.54 (1H, d), | From Example 366 using General |
| | 4.64 (1H, q), 4.45-4.41 (2H, m), 3.70-3.66 (2H, m) 3.42 (1H, obs sextet), 2.69-2.53 (2H, m), | Methods 1, 2 and 3. |
| | 2.24-1.94 (2H, m), 1.36 (3H, d), 0.88 (3H, t). MS: 422.2 (MH⁺), 98.4% d.e., 99.2% by LCMS, 1H NMR >95%. | Purification on silica (20 g 2:1 mix |
| | | normal/TLC). Eluent: 5% MeOH/EtOAc + |
| | | 0.1% NH₃. |
| | | 2.1M HCl/EtOAc/Et₂O gave 24 mg (13% |
| | | yield). |
| 410 | 1H NMR (270 MHz, MeOD-d₄): 7.56-7.35 (2H, m), 6.75 (1H, m), 6.35 (2H, m), 4.57 (1H, m), | From Example 367 using General |
| | 4.20 (4H, obs bs), 3.36 (1H, m), 2.63-2.42 (2H, m), 2.20-1.93 (2H, m), 1.32 (3H, d), 0.87 (3H, t). | Methods 1, 2 and 3. |
| | 99.0% by LCMS, 423.1 (MH⁺). 96.1% d.e., 1H NMR >95%. | Purification on silica (2 g 1:1 mix |
| | | normal/TLC). Eluent: 10% |
| | | MeOH/EtOAc + 1% NH₃. |
| | | 2M HCl/EtOAc/Et₂O gave 12 mg (9.4%). |
| 411 | 1H NMR (270 MHz, MeOD-d₄): 8.84 (2H, m), 7.69 (4H, m), 4.68 (1H, dd), 3.48 (1H, obs sextet), | From Example 368 using General |
| | 2.71-2.55 (2H, 2 × dd), 2.30-2.05 (2H, m), 1.39 (3H, d), 0.92 (3H, t). 95.5% by LCMS, 366.0 (MH⁺). | Methods 1, 2 and 3. |
| | 68.2% d.e., 1H NMR >85%. | Purification on silica (10 g 1:1 mix |
| | | normal/TLC). Eluent: 10% |
| | | MeOH/EtOAc + 0.2% NH₃. 2M |
| | | HCl/EtOAc/Et₂O gave 17 mg (19.6%). |
| 412 | 1H NMR (270 MHz, MeOD-d₄): 8.02-8.00 (1H, m), 7.88 (1H, ddd), 7.54-7.42 (2H, m), | From Example 369 using General |
| | 7.17-7.12 (2H, m), 4.63 (1H, dd), 3.69-3.60 (1H, m), 2.69-2.48 (2H, 2 × dd), 2.24-2.17 (1H, m), 2.07-2.00 (1H, | Methods 1, 2 and 3. |
| | m), 1.29 (3H, d), 0.92 (3H, t). 98.4% by LCMS, 366.1 (MH⁺). 99.9% d.e, 1H NMR >95%. | Purification on silica (20 g 1:1 mix |
| | | normal/TLC). Eluent: EtOAc/MeOH/2% aq |
| | | NH₃ in MeOH 95:5:2. |
| | | 2M HCl/EtOAc/Et₂O gave 63 mg (24%). |
| 413 | 1H NMR (270 MHz, MeOD-d₄): 8.02-7.95 (1H, m), 7.92-7.82 (1H, m), 7.54-7.43 (2H, m), | From Example 369 using General |
| | 7.17-7.11 (2H, m), 4.65-4.59 (1H, m), 3.47-3.41 (1H, m), 2.64-2.59 (2H, m), 2.18-2.00 (2H, m), 1.22 (3H, d), | Methods 1, 2 and 3. |
| | 0.92 (3H, t). | Purification on silica (20 g 1:1 mix |
| | 96.2% by LCMS, 366.1 (MH⁺). 98.3% d.e., 1H NMR >95%. | normal/TLC). Eluent: EtOAc/MeOH/2% aq |
| | | NH₃ in MeOH 95:5:2. |
| | | 2M HCl/EtOAc/Et₂O gave 58 mg (22%). |
| 414 | 96.8% by LCMS, 381.2 (MH⁺). | From Example 370 using General |
| | 56% S-diastereomer, 44% R-diastereomer by chiral HPLC. | Methods 1, 2 and 3. |
| | | Purification on silica (10 g 2:1 mix |
| | | normal/TLC). Eluent: 5-10% |
| | | MeOH/EtOAc + 0.1% NH_{3.} Isolated 44 mg |
| | | mixed diastereomers (14.6%). |
| 415 | 1H NMR (270 MHz, MeOD-d₄): 8.18 (1H, d), 7.61 (1H, dd), 7.50 (2H, m), 6.71 (1H, m), 4.32 (1H, | From Example 371 using General |
| | dd), 3.16-3.05 (1H, m), 2.41 (2H, d), 2.10 (3H, s), 2.06-1.76 (2H, m), 1.19 (3H, d), 0.86 (3H, t). | Methods 1, 2 and 3. |
| | 88.5% by LCMS, 423.2 (MH⁺). 56% S-diastereomer, 44% R-diastereomer by chiral HPLC. 1H | Purification on silica (6 g 2:1 mix |
| | NMR >85%. | normal/TLC). Eluent: 5-10% |
| | | MeOH/EtOAc + 0.1% NH_{3.} Isolated 26 mg |
| | | mixed diastereomers (42.3%). |
| 416 | 1H NMR (270 MHz, MeOD-d₄): 7.91 (1H, m), 7.81-7.76 (2H, m), 7.57 (1H, dd), 7.50 (1H, dd), | From Example 372 using General |
| | 7.04-6.98 (2H, m), 6.82 (1H, d), 4.65 (1H, dd), 3.66-3.55 (1H, obs sextet), 2.69-2.51 (2H, 2 × dd), | Methods 1, 2 and 3. |
| | 2.26-2.19 (1H, m), 2.07-1.98 (1H, m), 1.36 (3H, d), 0.91 (3H, t). 99.2% by LCMS, 431.2 (MH⁺). | Purification on silica (20 g 1:1 mix |
| | 98.9% d.e., 1H NMR >95%. | normal/TLC). Eluent: EtOAc/MeOH/2% aq |
| | | NH₃ in MeOH 90:8:2. 2M HCl/EtOAc/Et₂O |
| | | gave 33.7 mg (32%). |
| 417 | 1H NMR (270 MHz, MeOD-d₄): 7.84 (1H, m), 7.79-7.74 (2H, m), 7.58 (1H, dd), 7.49 (1H, dd), | From Example 372 using General |
| | 7.01-6.96 (2H, m), 6.76 (1H, d), 4.64 (1H, dd), 3.50-3.38 (1H, m), 2.66-2.51 (2H, 2 × dd), | Methods 1, 2 and 3. |
| | 2.20-2.02 (2H, m), 1.36 (3H, d), 0.90 (3H, t). 97.4% by LCMS, 431.2 (MH⁺). 96.9% d.e., 1H NMR >95%. | Purification on silica (20 g 1:1 mix |
| | | normal/TLC). Eluent: EtOAc/MeOH/2% aq NH₃ in MeOH |
| | | 90:8:2. |
| | | 2M HCl/EtOAc/Et₂O gave 25.9 mg (24%). |
| 418 | 1H NMR (270 MHz, MeOD-d₄): 7.62-7.43 (2H, m), 7.30-7.15 (3H, m), 4.62 (1H, dd), | From Example 373 using General |
| | 3.68-3.55 (1H, m), 2.56 (2H, m), 2.30-2.12 (1H, m), 2.10-1.95 (1H, m), 1.33 (3H, d), 0.90 (3H, t). 97.1% by | Methods 1, 2 and 3. |
| | LCMS, 426.2 (MH⁺). 99.8% d.e., 1H NMR >95%. | Purification on silica (10 g 1:1 mix |
| | | normal/TLC). Eluent: 5-10% |
| | | MeOH/DCM + 0.1% NH_{3.} |
| | | 2M HCl/DCM/Et₂O gave 15 mg (4.5%). |
| 419 | 1H NMR (270 MHz, MeOD-d₄): 7.61-7.46 (2H, m), 7.28-7.18 (3H, m), 4.63 (1H, dd), | From Example 373 using General |
| | 3.46-3.36 (1H, m), 2.58 (2H, m), 2.25-2.02 (2H, m), 1.35 (3H, d), 0.89 (3H, t). 98.8% by LCMS, 426.2 (MH⁺). | Methods 1, 2 and 3. |
| | 88.0% d.e., 1H NMR >95%. | Purification on silica (10 g 1:1 mix |
| | | normal/TLC). Eluent: 5-10% |
| | | MeOH/DCM + 0.1% NH_{3.} |
| | | 2M HCl/DCM/Et₂O gave 55 mg (16.4%). |
| 420 | 1H NMR (270 MHz, MeOD-d₄): 7.60-7.49 (2H, m), 7.28-7.09 (3H, m), 4.65 (1H, dd), | From Example 374 using General |
| | 3.68-3.52 (1H, obs sextet), 2.90 (3H, s), 2.69-2.51 (2H, 2 × dd), 2.31-2.18 (1H, m), 2.12-1.98 (1H, m), | Methods 1, 2 and 3. |
| | 1.35 (3H, d), 0.90 (3H, t). | Purification on silica (50 g 1:1 mix |
| | 89.7% by LCMS, 440.2 (MH⁺). 99.6% d.e., 1H NMR >95%. | normal/TLC). Eluent: |
| | | 5% MeOH/CH₂Cl₂ + 0.1% NH₃. |
| | | 2M HCl/EtOAc/Et₂O gave 82 mg (19%). |
| 421 | 1H NMR (270 MHz, MeOD-d₄): 7.59-7.48 (2H, m), 7.27-7.13 (3H, m), 4.62 (1H, dd), | From Example 374 using General |
| | 3.44-3.35 (1H, m), 2.90 (3H, s), 2.68-2.52 (2H, m), 2.23-2.00 (2H, m), 1.35 (3H, d), 0.89 (3H, t). | Methods 1, 2 and 3. |
| | 97.9% by LCMS, 440.2 (MH⁺). 74.7% d.e., 1H NMR >95%. | Purification on silica (50 g 1:1 mix |
| | | normal/TLC). Eluent: |
| | | 5% MeOH/CH₂Cl₂ + 0.1% NH₃. |
| | | 2M HCl/EtOAc/Et₂O gave 51 mg (12%). |
| 422 | 1H NMR (270 MHz, MeOD-d₄): 7.57 (1H, dd), 7.49 (1H, dd), 7.22 (1H, t), 7.09-7.03 (1H, m), | From Example 375 using General |
| | 6.94 (1H, m), 4.63 (1H, dd), 3.72 (4H, br s), 3.67-3.58 (3H, m), 3.40-3.33 (2H, m), 2.70-2.46 (2H, 2 × dd), | Methods 1, 2 and 3. |
| | 2.32-2.13 (1H, m), 2.12-1.90 (1H, m), 1.28 (3H, d), 0.90 (3H, t). 98.7% by LCMS, 496.3 (MH⁺). | Purification on silica (24 g 1:1 mix |
| | 99.9% d.e., | normal/TLC). Eluent: |
| | 1H NMR >95%. | 5% MeOH/CH₂Cl₂ + 0.1% NH₃. |
| | | 2M HCl/EtOAc/Et₂O gave 96 mg. |
| 423 | 1H NMR (270 MHz, MeOD-d₄): 7.57-7.45 (2H, m), 7.22 (1H, t), 7.12-7.06 (1H, m), 6.95 (1H, m), | From Example 375 using General |
| | 4.60 (1H, dd), 3.72 (4H, br s), 3.66-3.58 (2H, m), 3.50-3.20 (3H, m), 2.69-2.45 (2H, m), | Methods 1, 2 and 3. |
| | 2.25-2.02 (2H, m), 1.32 (3H, d), 0.90 (3H, t). | Purification on silica (24 g 1:1 mix |
| | 97.4% by LCMS, 496.3 (MH⁺). 95.7% d.e., 1H NMR >95%. | normal/TLC). Eluent: |
| | | 5% MeOH/CH₂Cl₂ + 0.1% NH₃. |
| | | 2M HCl/EtOAc/Et₂O gave 73 mg. |
| 424 | 1H NMR (270 MHz, MeOD-d₄): 7.59-7.47 (2H, m), 7.21 (1H, t), 7.09-7.03 (1H, m), 6.90 (1H, m), | From Example 376 using General |
| | 4.64 (1H, dd), 3.66-3.57 (1H, observed sextet), 3.08 (3H, s), 2.94 (3H, s), 2.68-2.51 (2H, 2 × dd), | Methods 1, 2 and 3. |
| | 2.27-2.17 (1H, m), 2.06-1.96 (1H, m), 1.35 (3H, d), 0.89 (3H, t). 94.7% by LCMS, 454.3 (MH⁺). | Purification on silica (50 g 2:1 mix |
| | 99.0% d.e., 1H NMR >95%. | normal/TLC). Eluent: EtOAc:MeOH:NH₃ |
| | | 95:5:0.1. |
| | | 2M HCl/EtOAc/Et₂O gave 48 mg (14%). |
| 425 | 1H NMR (270 MHz, MeOD-d₄): 7.57-7.46 (2H, m), 7.21 (1H, t), 7.11-7.05 (1H, m), 6.92 (1H, m), | From Example 376 using General |
| | 4.62 (1H, dd), 3.49-3.37 (1H, m), 3.07 (3H, s), 2.93 (3H, s), 2.67-2.50 (2H, 2 × dd), 2.23-2.03 (2H, | Methods 1, 2 and 3. |
| | m), 1.36 (3H, d), 0.88 (3H, t). 99.8% by LCMS, 454.3. 99.0% d.e., 1H NMR >95%. | Purification on silica (50 g 2:1 mix |
| | | normal/TLC). Eluent: EtOAc:MeOH:NH₃ |
| | | 95:5:0.1. |
| | | 2M HCl/EtOAc/Et₂O gave 52 mg (15%). |
| 426 | 1H NMR (270 MHz, MeOD-d₄): 8.65 (1H, s), 8.38 (1H, d), 8.09 (1H, s), 7.57-7.46 (2H, m), | From Example 380 using General |
| | 4.62-4.51 (1H, m), 3.60-3.56 (1H, m), 2.67-2.47 (2H, m), 2.25-1.98 (2H, m), 1.32 (3H, d), 0.91 (3H, t). | Methods 1, 2 and 3. |
| | 95.1% by LCMS, 367.2 (MH⁺), 99.8% d.e., 1H NMR >95% | Purification on silica (3 g 1:1 mix |
| | | normal/TLC). Eluent: 10% MeOH/EtOAc + |
| | | 0.2% NH₃. 2M HCl/EtOAc/Et₂O gave 8.5 mg |
| | | (4% yield). |
| 427 | 1H NMR (270 MHz, MeOD-d₄): 8.64 (1H, s), 8.37 (1H, d), 8.10-8.09 (1H, m), 7.55-7.45 (2H, m), | From Example 380 using General |
| | 4.71-4.62 (1H, m), 3.08-3.03 (1H, m), 2.54-2.51 (2H, m), 2.16-1.98 (2H, m), 1.30 (3H, d), 0.89 (3H, | Methods 1, 2 and 3. |
| | t). 98.8% by LCMS, 367.2 (MH⁺), 98.1% d.e., 1H NMR >95% | Purification on silica (3 g 1:1 mix |
| | | normal/TLC). Eluent: 10% MeOH/EtOAc + |
| | | 0.2% NH₃. 2M HCl/EtOAc/Et₂O gave 4.1 mg (2% yield). |
| 428 | 1H NMR (270 MHz, MeOD-d₄): 8.00 (1H, d), 7.78 (1H, d), 7.58-7.43 (2H, m), 4.64 (1H, dd), | From Example 382 using General |
| | 3.62 (1H, m), 2.70-2.50 (2H, m), 2.30-2.15 (1H, m), 2.11-1.94 (1H, m), 1.34 (3H, d), 0.90 (3H, t). | Methods 1, 2 and 3. |
| | 99.4% by LCMS, 382.2 (MH⁺). 99.8% d.e., 1H NMR >95%. | Purification on silica (6 g 1:1 mix |
| | | normal/TLC). Eluent: 5% |
| | | MeOH/DCM + 0.1% NH_{3.} |
| | | 2M HCl/EtOAc/Et₂O gave 7.3 mg (7.0%). |
| 429 | 1H NMR (270 MHz, MeOD-d₄): 7.99 (1H, d), 7.76 (1H, d), 7.55-7.43 (2H, m), 4.63 (1H, dd), | From Example 381 using General |
| | 3.40 (1H, m), 2.60 (2H, m), 2.26-1.96 (2H, m), 1.34 (3H, d), 0.89 (3H, t). | Methods 1, 2 and 3. |
| | 94.9% by LCMS, 382.2 (MH⁺). 1H NMR >90%. | Purification on silica (6 g 1:1 mix |
| | | normal/TLC). Eluent: 5% |
| | | MeOH/DCM + 0.1% NH_{3.} |
| | | 2M HCl/EtOAc/Et₂O gave 0.7 mg (0.7%). |
| 430 | 1H NMR (270 MHz, MeOD-d₄): 8.55 (2H, d), 7.55-7.39 (2H, m), 7.22 (1H, t), 4.57 (1H, dd), | From Example 382 using General |
| | 3.59-3.49 (1H, observed sextet), 2.55-2.43 (2H, 2 × dd), 2.18-2.10 (1H, m), 1.98-1.92 (1H, m), 1.26 (3H, | Methods 1, 2 and 3. |
| | d), 0.82 (3H, t). | Purification on silica (30 g 1:1 mix |
| | 98.1% by LCMS, 367.2 (MH⁺). 98.2% d.e., 1H NMR >95%. | normal/TLC). Eluent: |
| | | 10% MeOH/EtOAc + 0.1% NH₃. 2M |
| | | HCl/EtOAc/Et₂O gave 82 mg (32%). |
| 431 | 1H NMR (270 MHz, MeOD-d₄): 8.58-8.53 (2H, m), 7.48-7.40 (2H, m), 7.22 (1H, t), 4.57 (1H, dd), | From Example 382 using General |
| | 3.36-3.27 (1H, m), 2.67-2.50 (2H, m), 2.13-1.89 (2H, m), 1.26 (3H, d), 0.81 (3H, t). | Methods 1, 2 and 3. |
| | 96.8% by LCMS, 367.3 (MH⁺). 87.5% d.e., 1H NMR >95%. | Purification on silica (30 g 1:1 mix |
| | | normal/TLC). Eluent: |
| | | 10% MeOH/EtOAc + 0.1% NH₃. 2M |
| | | HCl/EtOAc/Et₂O gave 37 mg (15%). |
| 432 | 1H NMR (270 MHz, MeOD-d₄): 7.95 (2H, s), 7.46-7.31 (2H, m), 4.55 (1H, q), 3.55 (1H, obs | From Example 383 using General |
| | sextet), 2.62-2.39 (2H, m), 2.16-1.92 (2H, m), 1.26 (3H, d), 0.83 (3H, t). 94.7% by LCMS, | Methods 1, 2 and 3. |
| | 382.1 (MH⁺), 98.4% d.e., 1H NMR >95% | Purification on silica (11 g 1:1 mix |
| | | normal/TLC). Eluent: 10% MeOH/EtOAc + |
| | | 0.2% NH₃. 2M HCl/EtOAc/Et₂O gave 5.5 mg (3% yield). |
| 433 | 1H NMR (270 MHz, MeOD-d₄): 8.27 (2H, s), 7.48-7.39 (2H, m), 4.56 (1H, q), 3.38-3.29 (1H, m), | From Example 383 using General |
| | 2.52 (2H, d), 2.10-1.90 (2H, m), 1.27 (3H, d), 0.81 (3H, t). | Methods 1, 2 and 3. |
| | 95.0% by LCMS, 382.1 (MH⁺), 98.9% d.e., 1H NMR >95% | Purification on silica (11 g 1:1 mix |
| | | normal/TLC). Eluent: 10% MeOH/EtOAc + |
| | | 0.2% NH₃. 2M HCl/EtOAc/Et₂O gave 11.8 mg |
| | | (6% yield). |
| 434 | 1H NMR (270 MHz, MeOD-d₄): 7.78 (1H, d), 7.57-7.48 (3H, m), 7.38-7.25 (2H, m), 4.61 (1H, dd), | From Example 384 using General |
| | 3.59 (1H, obs sextet), 2.64-2.43 (2H, 2 × dd), 2.20-1.91 (2H, m), 1.29 (3H, d), 0.87 (3H, t). | Methods 1, 2 and 3. |
| | 99.0% by LCMS, 422.1 (MH⁺), 99.9% d.e., 1H NMR>95% | Purification on silica (10 g 1:1 mix |
| | | normal/TLC). Eluent: 5% MeOH/EtOAc + |
| | | 0.1% NH₃. |
| | | 2M HCl/EtOAc/Et₂O gave 14.5 mg (6% |
| | | yield). |
| 435 | 1H NMR (270 MHz, MeOD-d₄): 7.77 (1H, d), 7.56-7.49 (3H, m), 7.38-7.24 (2H, m), 4.61 (1H, dd), | From Example 384 using General |
| | 3.39 (1H, obs sextet), 2.61-2.46 (2H, m), 2.15-2.01 (2H, m), 1.29 (3H, d), 0.85 (3H, t). | Methods 1, 2 and 3. |
| | 99.8% by LCMS, 422.1 (MH⁺), 99.2% d.e., 1H NMR >95% | Purification on silica (10 g 1:1 mix |
| | | normal/TLC). Eluent: 5% MeOH/EtOAc + |
| | | 0.1% NH₃. |
| | | 2M HCl/EtOAc/Et₂O gave 30.5 mg (14% |
| | | yield). |
| 436 | LCMS: 437.2 (MH⁺), 27% purity | From Example 385 using General |
| | | Methods 1, 2 and 3. |
| | | Purification on silica (5 g 1:1 mix |
| | | normal/TLC). Eluent: 10% MeOH/EtOAc + |
| | | 0.2% NH₃ then purification on silica (6 g |
| | | 1:1 mix normal/TLC). Eluent: 5% |
| | | MeOH/EtOAc + 0.1% NH₃. Gave 50 mg |
| | | crude material. |
| 437 | 1H NMR (270 MHz, MeOD-d₄): 9.19 (1H, br s), 8.68 (1H, d), 8.62 (1H, d), 7.74-7.65 (2H, m), | From Example 386 using General |
| | 4.73 (1H, dd), 3.68-3.60 (1H, observed sextet), 2.69-2.56 (2H, 2 × dd), 2.30-2.23 (1H, m), 2.13-2.00 (1H, | Methods 1, 2 and 3. |
| | m), 1.37 (3H, d), 0.92 (3H, t). 96.1% by LCMS, 423.2. 99.3% d.e., 1H NMR >95%. | Purification on silica (20 g 1:1 mix |
| | | normal/TLC). Eluent: EtOAc/MeOH/2% aq |
| | | NH₃ in MeOH 90:10:2. 2M HCl/EtOAc/Et₂O gave 59 mg |
| | | (37%). |
| 438 | 1H NMR (270 MHz, MeOD-d₄): 9.20 (1H, br s), 8.67 (1H, d), 8.62 (1H, d), 7.73-7.64 (2H, m), | From Example 386 using General |
| | 4.72 (1H, dd), 3.49-3.39 (1H, observed sextet), 2.69-2.62 (2H, m), 2.25-2.10 (2H, m), 1.35 (3H, d), | Methods 1, 2 and 3. |
| | 0.92 (3H, t). | Purification on silica (20 g 1:1 mix |
| | 97.7% by LCMS, 423.2 (MH⁺). 95.7% d.e., 1H NMR >95%. | normal/TLC). Eluent: EtOAc/MeOH/2% aq |
| | | NH₃ in MeOH 90:10:2. 2M HCl/ |
| | | EtOAc/Et₂O gave 26 mg(16%). |
| 439 | 1H NMR (270 MHz, MeOD-d₄): 7.56-7.49 (2H, m), 4.60 (2H, q), 3.56 (1H, obs sextet), | From Example 387 using General |
| | 2.64-2.42 (5H, m), 2.22-1.91 (2H, m), 1.28 (3H, d), 0.83 (3H, t). 98.9% by LCMS, 387.1 (MH⁺), 99.9% d.e., | Methods 1, 2 and 3. |
| | 1H NMR >95 | Purification on silica (10 g 1:1 mix |
| | | normal/TLC). Eluent: 10% MeOH/EtOAc + |
| | | 0.2% NH₃. 2M HCl/EtOAc/Et₂O gave 22 mg (13% yield). |
| 440 | 1H NMR (270 MHz, MeOD-d₄): 7.55-7.47 (2H, m), 4.59 (1H, q), 3.32 (1H, obs sextet), 2.59 (3H, | From Example 387 using General |
| | s), 2.53-2.50 (2H, m), 2.14-1.93 (2H, m), 1.28 (3H, d), 0.81 (3H, t). 99.4% by LCMS, 387.1 (MH⁺), | Methods 1, 2 and 3.. Purification on silica |
| | 96.1% d.e., 1H NMR >95% | (10 g 1:1 mix normal/TLC). Eluent: 10% |
| | | MeOH/EtOAc + 0.2% NH₃. 2M |
| | | HCl/EtOAc/Et₂O gave 18 mg (10% yield). |
| 441 | 1H NMR (270 MHz, MeOD-d₄): 7.89 (1H, dd), 7.70-7.50 (3H, m), 7.28 (1H, d), 4.66 (1H, dd), | From Example 389 using General |
| | 3.65-3.55 (1H, m), 3.27 (6H, s), 2.63 (2H, s), 2.30-1.95 (2H, m), 1.37 (3H, d), 0.89 (3H, t). 99.6% by | Methods 1, 2 and 3.. Purification on silica |
| | LCMS, 409.2 (MH⁺). 99.2% d.e., 1H NMR >95% | (21 g 1:1 mix normal/TLC). Eluent: 10% |
| | | MeOH/EtOAc + 0.2% NH_{3.} 2M |
| | | HCl/EtOAc/Et₂O gave 39 mg (12.7%). |
| 442 | 1H NMR (270 MHz, MeOD-d₄): 7.88 (1H, dd), 7.62-7.52 (3H, m), 7.26 (1H, d), 4.64 (1H, dd), | From Example 389 using General |
| | 3.47-3.36 (1H, m), 3.26 (6H, s), 2.61 (2H, m), 2.27-2.00 (2H, m), 1.37 (3H, d), 0.89 (3H, t). 99.6% by | Methods 1, 2 and 3.. Purification on silica |
| | LCMS, 409.2 (MH⁺). 97.2% d.e., 1H NMR >95% | (21 g 1:1 mix normal/TLC). Eluent: 10% |
| | | MeOH/EtOAc + 0.2% NH_{3.} 2M |
| | | HCl/EtOAc/Et₂O gave 16 mg (5.2%). |
| 443 | 1H NMR (270 MHz, MeOD-d₄): 7.89 (2H, d), 7.64-7.45 (2H, m), 6.96 (2H, d), 4.65 (1H, m), | From Example 390 using General |
| | 3.56 (1H, m), 2.70-2.47 (2H, m), 2.30-2.14 (1H, m), 2.11-1.94 (1H, m), 1.34 (3H, d), 0.91 (3H, t). 94.9% | Methods 1, 2 and 3.. Purification on silica |
| | by LCMS, 408.3 (MH⁺). 99.7% d.e., 1H NMR >95%. | (20 g 2:1 mix normal/TLC). Eluent: 5-10% |
| | | MeOH/DCM + 0.1% NH_{3.} |
| | | 2M HCl/DCM/Et₂O gave 5.0 mg (4.1%). |
| 444 | 1H NMR (270 MHz, MeOD-d₄): 7.88 (2H, d), 7.62-7.46 (2H, m), 6.97 (2H, d), 4.63 (1H, dd), | From Example 390 using General |
| | 3.42 (1H, m), 2.68-2.48 (2H, m), 2.26-2.00 (2H, m), 1.50 (3H, d), 0.90 (3H, t). 98.8% by LCMS, | Methods 1, 2 and 3.. Purification on silica |
| | 408.3 (MH⁺). 95.8% d.e., 1H NMR >95%. | (20 g 2:1 mix normal/TLC). Eluent: 5-10% |
| | | MeOH/DCM + 0.1% NH_{3.} |
| | | 2M HCl/DCM/Et₂O gave 12.1 mg (10.0%). |
| 445 | 1H NMR (270 MHz, MeOD-d₄): 9.17 (1H, d), 9.00 (1H, dd), 8.68 (1H, ddd), 7.98 (1H, dd), | From Example 393 using General |
| | 7.90 (1H, t), 7.63 (1H, d), 4.10 (1H, d), 3.86-3.76 (1H, observed sextet), 2.71-2.53 (2H, 2 × dd), | Methods 1, 2 and 3.. Purification on silica |
| | 1.59-1.53 (1H, m), 1.38 (3H, d), 1.07-0.91 (1H, m), 0.89-0.63 (2H, m), 0.49-0.31 (1H, m). 99.6% by | (25 g 1:1 mix normal/TLC). Eluent: EtOAc/MeOH/2% aq NH₃ in |
| | LCMS, 390.2 (MH⁺). 99.1% d.e., 1H NMR >95%. | MeOH 90:8:2 2M |
| | | HCl/EtOAc/Et₂O gave 59 mg (18%). |
| 453 | 1H NMR (270 MHz, MeOD-d₄): 7.88 (1H, dd), 7.65-7.55 (3H, m), 7.06 (1H, d), 4.08 (1H, d), | From KI-28 using General Method 3. |
| | 3.76 (1H, m), 2.72-2.53 (2H, m), 1.60-1.45 (1H, m), 1.38 (3H, d), 1.04-0.90 (1H, m), 0.84-0.62 (2H, m), | Purification on silica (40 g 1:1 mix |
| | 0.42-0.32 (1H, m). 85.8% by LCMS, 97.1% d.e., 1H NMR >95%. | normal/TLC). Eluent: |
| | | 5% MeOH/DCM + 0.1% NH_{3.} |
| | | 2M HCl/DCM/Et₂O gave 19 mg. |
| 454 | 1H NMR (270 MHz, MeOD-d₄): 7.89 (1H, dd), 7.64 (1H, d), 7.62-7.52 (2H, m), 7.06 (1H, d), | From KI-28 using General Method 3. |
| | 3.95 (1H, d), 3.54-3.40 (1H, m), 2.63 (2H, d), 1.69-1.54 (1H, m), 1.35 (3H, d), 1.01-0.88 (1H, m), | Purification on silica (40 g 1:1 mix |
| | 0.80-0.64 (2H, m), 0.49-0.36 (1H, m). 100% by LCMS, 393.2 (MH⁺). 94.8% d.e., 1H NMR >95%. | normal/TLC). Eluent: 5% |
| | | MeOH/DCM + 0.1% NH_{3.} |
| | | 2M HCl/DCM/Et₂O gave 24 mg (8.7%). |
| 455 | 1H NMR (270 MHz, MeOD-d₄): 8.40 (1H, d), 8.10 (1H, d), 7.62 (2H, m), 7.41 (1H, dd), 4.08 (1H, | From Example 398 via intermediates KI- |
| | d), 3.83-3.70 (1H, m), 2.71-2.46 (2H, m), 1.60-1.46 (1H, m), 1.36 (3H, d), 1.02-0.90 (1H, m), | 29 and KI-30 using General Methods 1, 2 |
| | 0.84-0.65 (2H, m), 0.46-0.34 (1H, m). 99.8% by LCMS, 421.2 (MH⁺). 98.7% d.e., 1H NMR >95%. | and 3. Purification on silica (40 g 2:1 mix |
| | | normal/TLC). Eluent: 5-7% MeOH/DCM + |
| | | 0.1% NH_{3.} 2M HCl/DCM/Et₂O gave 38 mg |
| | | (10.5%). |
| 456 | 1H NMR (270 MHz, MeOD-d₄): 8.42 (1H, d), 8.10 (1H, d), 7.64-7.56 (2H, m), 7.42 (1H, dd), | From Example 398 via intermediates KI- |
| | 4.00 (1H, d), 3.57-3.44 (1H, m), 2.63 (2H, m), 1.68-1.55 (1H, m), 1.36 (3H, d), 1.01-0.90 (1H, m), | 29 and KI-30 using General Methods 1, 2 |
| | 0.80-0.66 (2H, m), 0.52-0.36 (1H, m). 100% by LCMS, 421.2 (MH⁺). 90.1% d.e., 1H NMR >95%. | and 3. Purification on silica (40 g 2:1 mix |
| | | normal/TLC). Eluent: 5-7% MeOH/DCM + |
| | | 0.1% NH_{3.} 2M HCl/DCM/Et₂O gave 20 mg |
| | | (5.5%). |
| 457 | 1H NMR (400 MHz, Me-d3-OD): 7.64-7.52 (2H, m), 7.41-7.29 (2H, m), 7.17-7.06 (1H, m), | As Example 277 using Example 346 in |
| | 6.89 (2H, d), 4.64 (1H, d), 3.50 (2H, t), 3.47-3.39 (1H, m), 3.08 (2H, t), 2.80 (1H, dd), 2.73 (1H, dd), | step1. Separation of diastereomers |
| | 2.19-2.06 (1H, m), 1.72-1.57 (2H, m), 1.57-1.42 (1H, m), 1.36 (3H, d), 1.24-1.10 (1H, m), 1.04 (3H, | by preparative hplc. |
| | t), 0.88 (3H, t). m/z: 450.2 (Molecular ion) | |
| 458 | 1H NMR (400 MHz, Me-d3-OD): 8.07 (1H, d), 7.99-7.82 (1H, m), 7.68-7.49 (3H, m), 4.66 (1H, dd), | Prepared analogously to Example 277 |
| | 3.49-3.42 (1H, m), 2.73-2.49 (2H, m), 2.27-2.05 (5H, m), 1.46-1.37 (3H, m), 0.92 (3H, t). m/z: | using benzyamine from Example 355 in |
| | 423 (Molecular ion) | step 1 |
| 459 | 1H NMR (400 MHz, Me-d3-OD): 7.55 (2H, d), 7.40-7.29 (2H, m), 7.10 (1H, t), 6.90 (2H, d), | Prepared analogously to Example 88 |
| | 4.00 (1H, d), 3.56-3.43 (1H, m), 2.64 (2H, d), 1.60 (1H, s), 1.35 (3H, d), 1.02-0.89 (1H, m), | using benzyamine from Example 463 |
| | 0.81-0.67 (2H, m), 0.50-0.38 (1H, m). m/z: 376 (Molecular ion) | |
| 460 | 1H NMR (400 MHz, Me-d3-OD): 7.92 (1H, dd), 7.61 (1H, d), 7.60-7.54 (2H, m), 7.12 (1H, d), | Example 460 |
| | 4.66 (1H, dd), 2.67 (1H, d), 2.63 (1H, d), 2.25-2.07 (2H, m), 1.50 (3H, s), 1.39 (3H, s), 0.91 (3H, t). m/z: | |
| | 395 (Molecular ion) | |
| 461 | m/z: 296/298 (Molecular ion) | As for 112 using 4-nitro-2-chloropyridine |
| 462 | m/z: 310 (Molecular ion) | As for Example 132, step 1 using Key |
| | | Intermediate 3a and 6-(tert- |
| | | butoxycarbonylmethyl-amino)-pyridine-3- |
| | | boronic acid followed by Key Intermediate |
| | | 1, Step 6 |
| 463 | m/z: 274 (Fragment: [M + H − NH3]⁺) | As for example 88 to step 6 but using a |
| | | solution of cyclopropylmagnesium |
| | | bromide in step 5. |
| 464 | m/z: 376 (Molecular ion) | As for example 88 but using a solution of |
| | | cyclopropylmagnesium bromide in step 5. |
| 465 | m/z: 261 (Fragment: [M + H − NH3]⁺) | As for example 88 to step 6 but using a |
| | | solution of vinylmagnesium bromide in |
| | | step 5. |
| 466 | | Made using methods described herein |
| 467 | | Made using methods described herein |
| 468 | | Made using methods described herein |
| 469 | | Made using methods described herein |
| 470 | | Made using methods described herein |
| 471 | | Made using methods described herein |
| 472 | | Made using methods described herein |

### BIOLOGICAL ACTIVITY

### Example A

### HCV NS3 Protease Assay

### NS3 Protease Assay

The HCV NS3 protease functions have been extensively studied and are considered as potential targets for antiviral therapy: see for example the many references listed in the introductory section of this application. Therefore, the activity of the compounds of the invention as anti-HCV agents was assessed using a full length HCV NS3 protease.

The protease activity of the full length NS34a was measured using a FRET-based assay utilizing a peptide substrate derived from the NS4AB cleavage site (Anaspec) and labelled at one end with a quencher (QXL520) and at the other with a fluorophore (5-FAMsp). NS34a (produced in-house by literature methods) was incubated with test compounds and peptide substrate in 50 mM Tris pH8, 20 mM DTT, 1% CHAPS, 10% glycerol and 5% DMSO. The reaction was followed by monitoring the change in fluorescence on a Molecular Devices Gemini plate reader for 30 minutes at room temperature. Initial rates were calculated from the progress curves using SoftMax Pro (Molecular Devices). IC₅₀ values were then calculated from replicate curves using Prism GraphPad software.

The activities of compounds having IC₅₀ values of 10 μM or less and compounds exhibiting at least 40% inhibition at a concentration of 3 μM or lower are set out in the table below. In the table, “Ex” refers to the Example in which the compound is described.

| | Activity |
|---|---|
| | IC₅₀ (μM) or % |
| Ex. No. | inhibition |
| 1 | 2.5 |
| 2 | 2.2 |
| 3 | 2.1 |
| 4 | 7 |
| 5 | 2 |
| 8 | 0.33 |
| 9 | 40.5% @ 0.01 μM |
| 10 | 1.8 |
| 11 | 2.2 |
| 12 | 1.2 |
| 13 | 3.1 |
| 14 | 1.7 |
| 16 | 4 |
| 19 | 9.8 |
| 22 | 3.2 |
| 27 | 1 |
| 29 | 4.4 |
| 31 | 7.1 |
| 32 | 0.88 |
| 34 | 4 |
| 37 | 5.6 |
| 38 | 9.7 |
| 40 | 6.3 |
| 41 | 7.1 |
| 43 | 6.6 |
| 44 | 4.7 |
| 45 | 1.7 |
| 49 | 3.2 |
| 52 | 1.8 |
| 53 | 5 |
| 54 | 1.4 |
| 56 | 1.1 |
| 58 | 1 |
| 59B | 2.1 |
| 62 | 7.2 |
| 64 | 0.43 |
| 65 | 2.1 |
| 66 | 0.75 |
| 67 | 3.4 |
| 69 | 6 |
| 70 | 0.73 |
| 71 | 0.95 |
| 72B | 0.7 |
| 75 | 3.3 |
| 76 | 2.9 |
| 77 | 0.59 |
| 78 | 1.2 |
| 79 | 0.31 |
| 81 | 0.39 |
| 82 | 1.9 |
| 83 | 6.7 |
| 84 | 0.45 |
| 88 | 0.1 |
| 89 | 3.9 |
| 91 | 2.4 |
| 92 | 0.91 |
| 93 | 0.79 |
| 95B | 0.41 |
| 96 | 6.7 |
| 97 | 0.84 |
| 98 | 1.6 |
| 100 | 1.1 |
| 101 | 1.3 |
| 107 | 0.22 |
| 108B | 1.9 |
| 110 | 2.6 |
| 111 | 0.63 |
| 112 | 0.16 |
| 113 | 0.68 |
| 114 | 1 |
| 115 | 1.8 |
| 116 | 0.13 |
| 118 | 1.7 |
| 119 | 0.32 |
| 121 | 0.21 |
| 122 | 1 |
| 123 | 1.6 |
| 124 | 0.11 |
| 125 | 0.23 |
| 127 | 0.11 |
| 128 | 1.2 |
| 129 | 0.36 |
| 130 | 1.3 |
| 131A | 1.2 |
| 132B | 0.52 |
| 133 | 0.7 |
| 134B | 0.084 |
| 135 | 9.7 |
| 136 | 1.1 |
| 137 | 0.45 |
| 138 | 0.63 |
| 139 | 1.3 |
| 140 | 1.3 |
| 141 | 2.8 |
| 143 | 0.54 |
| 144 | 0.4 |
| 145A | 7.4 |
| 146 | 3.1 |
| 147 | 0.11 |
| 148 | 1.7 |
| 149 | 3 |
| 150 | 2 |
| 151 | 3.1 |
| 152 | 6.9 |
| 154 | 0.18 |
| 155 | 1.7 |
| 156 | 6.2 |
| 157 | 2.1 |
| 158 | 0.42 |
| 162 | 6.8 |
| 163 | 2.5 |
| 164 | 0.27 |
| 166 | 2.1 |
| 167 | 6.1 |
| 168 | 3.7 |
| 169 | 4.8 |
| 170 | 2.3 |
| 171 | 7.7 |
| 172 | 3.7 |
| 173 | 3.5 |
| 174 | 6.2 |
| 176 | 1.7 |
| 178 | 2.2 |
| 179 | 0.61 |
| 180 | 3.7 |
| 181 | 2.3 |
| 182 | 5.7 |
| 183 | 1.2 |
| 184 | 6.1 |
| 185 | 1.5 |
| 186 | 0.039 |
| 187 | 6.9 |
| 188 | 2.7 |
| 189 | 8.6 |
| 190 | 1.8 |
| 192 | 3.1 |
| 193 | 0.96 |
| 194 | 3.1 |
| 196 | 0.42 |
| 197 | 2.6 |
| 198 | 1.6 |
| 199 | 3.8 |
| 201 | 0.2 |
| 202 | 5.4 |
| 203 | 1.6 |
| 204 | 0.72 |
| 207 | 0.21 |
| 208 | 3.4 |
| 209 | 4.9 |
| 210 | 0.16 |
| 211 | 0.072 |
| 212 | 0.82 |
| 213 | 0.13 |
| 215 | 0.61 |
| 216 | 0.97 |
| 218 | 8.2 |
| 219 | 0.22 |
| 220 | 4.6 |
| 221 | 0.43 |
| 222 | 0.37 |
| 223 | 0.081 |
| 224 | 65.5% @ 0.01 μM |
| 225B | 0.73 |
| 226 | 0.2 |
| 227 | 2.2 |
| 228 | 1.6 |
| 229 | 1.7 |
| 230 | 0.17 |
| 232 | 1.8 |
| 233 | 0.11 |
| 234 | 57% @ 0.03 μM |
| 235 | 0.43 |
| 236 | 50% @ 3 μM |
| 237 | 0.087 |
| 238B | 58% @ 0.03 μM |
| 239 | 41% @ 0.03 μM |
| 240 | 42% @ 0.01 μM |
| 241 | 47% @ 0.01 μM |
| 242 | 3.9 |
| 244 | 0.47 |
| 246 | 0.17 |
| 249 | 0.38 |
| 250 | 0.95 |
| 251 | 0.061 |
| 252 | 1.9 |
| 253 | 0.41 |
| 255 | 0.23 |
| 260 | 0.17 |
| 261 | 1.2 |
| 262 | 5.9 |
| 263 | 0.39 |
| 264 | 4.6 |
| 265 | 1.1 |
| 266C | 0.087 |
| 267 | 0.48 |
| 270 | 1.6 |
| 271 | 0.26 |
| 272 | 0.88 |
| 273B | 2.3 |
| 275 | 0.41 |
| 277 | 0.11 |
| 278 | 3.9 |
| 280 | 5.3 |
| 281 | 10 |
| 282 | 0.73 |
| 283 | 0.75 |
| 284 | 1.4 |
| 285 | 2.4 |
| 286 | 3 |
| 287 | 0.44 |
| 288 | 1.4 |
| 289 | 1.1 |
| 290 | 3.4 |
| 313 | 0.15 |
| 315 | 3.5 |
| 316 | 0.4 |
| 317 | 0.45 |
| 318 | 6.4 |
| 319 | 3.5 |
| 321 | 1.6 |
| 322 | 0.27 |
| 323 | 3.1 |
| 324 | 1.8 |
| 326 | 3.9 |
| 328 | 1.4 |
| 329 | 57% @ 0.03 μM |
| 330 | 1.1 |
| 331 | 49.5% @ 0.03 μM |
| 333 | 0.061 |
| 336 | 1 |
| 337 | 7.6 |
| 338 | 2 |
| 339 | 65% @ 0.01 μM |
| 340 | 0.08 |
| 342 | 2.7 |
| 343 | 6.3 |
| 344 | 1.5 |
| 345 | 1.8 |
| 346 | 5 |
| 347 | 2.7 |
| 348 | 10 |
| 350 | 2.6 |
| 351 | 6.4 |
| 352 | 0.32 |
| 356 | 6.4 |
| 357 | 5.7 |
| 358 | 0.91 |

The compounds of Examples 6, 17, 18, 20, 21, 23, 24, 28, 30, 33, 35, 36, 39, 42, 46, 48, 50, 51, 57, 59A, 60, 61, 73, 74A, 74B, 80, 85, 87B, 90, 94, 99, 102, 105B, 117, 120, 126, 131B, 142, 153, 159, 160, 161, 165, 175, 177, 191, 205, 206, 214, 217, 231, 236, 247, 248, 254, 257, 258, 259, 268, 269, 276A, 276B, 291, 292, 294, 299, 304, 307, 308, 314, 320, 325, 327, 332, 334, 335, 341, 353, 354, 355 and 359 all have IC₅₀ values of 10-150 μM against the protease activity of the full length NS34a in the above assay or demonstrate at least 40% inhibition of protease activity of the full length NS34a at a concentration of 100 μM in the above assay.

The results demonstrate that compounds of the invention are good inhibitors of the protease activity of the full length NS34a of HCV and should therefore exhibit good antiviral activity.

### Example B

### Replicon Assay

The activities of compounds of the invention against HCV in a cellular environment were analysed using a replicon assay as described below.

Thus, Huh-7 cells persistently infected with an HCV-RNA construct (Bartenschlager, R. Hepatitis C replicons: potential role for drug development. Nature Rev. Drug Discov. 1, 911-916 (2002)) comprising: 5′ and 3′ non-translated regions (NTR); the non-structural genes NS3 to NS5b; as well as the G418 drug resistance gene, neomycin, (for selection of cells carrying HCV replicon RNA) fused to the firefly Luciferase reporter gene (pFK13889luc-ubi-neoNS3-3′ET), were used to determine the cell based antiviral activity of compounds using luciferase activity as an indirect readout of HCV RNA load. In this assay 4×10⁻³ huh-7 cells persistently infected with the HCV subgenomic replicon construct above were platedwell in a 96 well tissue culture plate. The cells were allowed to attach overnight in DMEM medium supplemented with 10% FBS 1% NEAA, and 250 μgml gentamicin. The following day the medium was replaced with 200 μlwell of fresh medium as described above lacking gentamicin. Semilog dilutions of compounds in medium were then added to triplicate wells (non-edge) of the cultured cells to give a 0.1% DMSO final concentration. Plates were then incubated at 37° C. in an atmosphere of 5% CO2 and air for 72 h. Following the 72 h incubation, compound CC50 values were determined by adding 20 μl of Alamar Blue™ (Biosource International, Camarillo, Calif., USA) to each well and incubating for 6 h at 37° C. in an atmosphere of 5% CO₂ and air. The plate was then read at 535 nm (excitation) and 590 nm (emission) on a SpectraMax Gemini reader (Molecular Devices) to determine the number of viable cells by measuring the conversion of rezasurin (Alamar blue) to resorufin in response to mitochondrial activity. In order to determine the antiviral effect of these compounds EC₅₀ values were determined by measuring the luciferase activity of the cells. Alamar blue solution was removed from the wells and replaced with 100 μlwell of medium along with 100 μlwell of Bright-Glo reagent and incubated at room temperature for 5 minutes before transferring 100 μlwell to a white bottom 96 well plate to read in a luminometer as described in the Bright-Glo Luciferase Assay System protocol (promega). The activities of compounds of the invention in the above assay, as defined by the EC₅₀ values (EC₅₀ luciferase readout), are set out in the table below.

| Ex. | Activity | Ex. | Activity | Ex. | Activity | Ex. | Activity |
|---|---|---|---|---|---|---|---|
| No. | EC₅₀ (μM) | No. | EC₅₀ (μM) | No. | EC₅₀ (μM) | No. | EC₅₀ (μM) |
| 1 | 15 | 2 | 2.4 | 3 | 22 | 4 | 13 |
| 5 | 0.13 | 6 | 0.14 | 8 | 10 | 9 | 10 |
| 11 | 0.8 | 12 | 10 | 13 | 2 | 14 | 2 |
| 16 | 0.58 | 17 | 2.8 | 18 | 1.6 | 19 | 10 |
| 21 | >30 | 22 | 6.6 | 24 | 18 | 27 | 0.26 |
| 29 | 0.47 | 32 | 8 | 34 | 1.2 | 44 | 9.9 |
| 45 | 9.1 | 49 | >30 | 50 | 23 | 52 | 9.4 |
| 53 | 26 | 56 | 11 | 57 | 29 | 58 | 27 |
| 59B | 3 | 60 | 3 | 61 | >30 | 62 | >30 |
| 64 | >25:18 | 65 | 28:12 | 66 | >30:>30 | 67 | >30:>30 |
| 70 | 33:27 | 71 | >30:>30 | 72B | 8.1:0.26 | 73 | >30:>30 |
| 74A | >30:>30 | 75 | 9.6:4.8 | 76 | 15:6 | 77 | 13:12 |
| 78 | 11:9.6 | 79 | >30:>10 | 80 | >10:>10 | 81 | 13:11 |
| 82 | 17:0.79 | 83 | 26:17 | 84 | >30:18 | 85 | >30:18 |
| 87B | >10:3.9 | 88 | >10:0.4 | 89 | 13:1.8 | 90 | 9.6:0.36 |
| 91 | >10:>10 | 92 | >30:>30 | 93 | 12:1.8 | 95B | 19:0.16 |
| 96 | 6.3:0.24 | 97 | >10:>10 | 98 | >10:>10 | 100 | >30:>18 |
| 101 | 18:0.23 | 107 | >10:>10 | 110 | 4.6:2.3 | 112 | >10:>10 |
| 114 | >10:>10 | 115 | >10:>10 | 116 | 27:0.19 | 118 | 4.9:4.4 |
| 119 | 3:3.4 | 123 | >10:4.7 | 124 | >10:0.28 | 125 | 28:0.38 |
| 127 | >30:0.014 | 128 | >10:>10 | 131A | >10:>10 | 134B | >10:0.4 |
| 137 | 5.4:7.8 | 143 | 9.9:6.2 | 145A | 7.6:9.5 | 154 | >10:17 |
| 155 | >10:>10 | 163 | 0.75:0.94 | 164 | 3:2.3 | 165 | >10:>10 |
| 178 | 19:8 | 186 | >30:0.13 | 196 | >10:0.27 | 201 | >10:>10 |
| 207 | >10:>10 | 210 | none:0.2 | 211 | >30:0.066 | 212 | >10:>10 |
| 213 | 1.7:5.2 | 216 | >10:>10 | 219 | >10:0.32 | 221 | >30:>30 |
| 222 | >10:>10 | 223 | >30:>30 | 224 | 4.5:0.0088 | 225B | 0.82:0.68 |
| 226 | 3:2.9 | 230 | >10:0.48 | 233 | 31:0.079 | 234 | >3:7.3 |
| 235 | 2.4:0.6 | 236 | 6.2:1.7 | 237 | >1:0.031 | 238B | 3.8:0.03 |
| 239 | 6:0.97 | 240 | 3.7:8.9 | 241 | 24:0.98 | 242 | 3.4:0.89 |
| 243 | >10:3.8 | 244 | 9.2:0.099 | 246 | >10:>10 | 249 | 1.5:4.6 |
| 250 | 4.3:1 | 251 | 5:1.8 | 252 | 2.8:0.88 | 253 | 7.4:0.13 |
| 255 | >10:0.012 | 260 | 2.7:0.043 | 263 | 4.5:1.6 | 264 | 0.7:0.6 |
| 265 | 0.95:0.47 | 266C | >1:0.023 | 267 | >10:>10 | 271 | 2.1:0.34 |
| 272 | 6.3:5.3 | 273B | 3:0.76 | 275 | >10:>10 | 277 | 1.4:1 |
| 280 | 5.1:0.4 | 281 | 3.9:0.79 | 282 | 3.2:0.37 | 283 | 3.8:0.27 |
| 284 | >10:>10 | 287 | 20:6.5 | 288 | 11:1.4 | 289 | 0.84:0.18 |
| 290 | 3.7:10 | 301 | >30:26 | 302 | >30:6.9 | 313 | >10:>10 |
| 315 | >10:>10 | 316 | >10:>10 | 317 | >10:2.7 | 319 | 6.7:8.6 |
| 322 | >10:1.2 | 324 | >10:>10 | 325 | >10:>10 | 329 | 4.9:4 |
| 331 | >10:>10 | 333 | >30:0.057 | 400 | >3:3 | 402 | >10:>10 |
| 404 | >3:0.023 | 405 | >3:0.3 | 407 | >3:0.11 | 409 | >3:0.02 |
| 410 | >3:0.31 | 413 | >3:3 | 417 | >3:0.91 | 419 | >3:0.82 |
| 421 | >3:0.4 | 423 | >3:1.4 | 425 | >3:0.35 | 429 | >3:3 |
| 431 | >3:>3 | 433 | >3:1.6 | 435 | >3:3 | 438 | >3:>3 |
| 444 | >3:0.82 | 446 | >3:0.071 | 450 | >3:0.009 | 454 | >3:0.16 |
| 456 | >3:>3 | 458 | >30:3.5 | 459 | >3:0.63 | 460 | >3:0.13 |
| 464 | >3:0.63 | 468 | >3:>3 | 472 | >3:>3 | 473 | >3:0.13 |

### Example C

### HCV Helicase Assay

The HCV NS3 NTPasehelicase functions have been extensively studied and are considered as potential targets for antiviral therapy: see for example the many references listed in the introductory section of this application. Therefore, the activity of the compounds of the invention as anti-HCV agents was assessed using an HCV helicase assay.

The helicase assay used is based on the method of Boguszewka-Chachulska, (Febs Letters 567 (2004) 253-258). The assay utilises a DNA substrate, labelled on the 5′ end with Cy3 (Cy3-TAGTACCGCCACCCTCAGAACCTTTTTTTTTTTTT) annealed to a DNA oligo labelled on the 3′ end with Black Hole Quencher (GGTTCTGAGGGTGGCGGTACTA-BHQ-2). When the labelled strands are separated, the fluorescence increases and the free quencher strand is prevented from re-annealing by binding to a complementary capture strand (TAGTACCGCCACCCTCAGAACC). Each well contains 50 nM HCV NS3 enzyme, 0.25 nM Fluorescence quench annealed DNA oligos, 3.125 uM Capture strand, 2 mM ATP in a buffer containing 30 mM Tris, pH7.5, 10 mM MnCl2, 0.1% Tween 20, 5% glycerol, 0.05% sodium azide. Fluorescence is continuously monitored at 580 nm after excitation at 550 nm.

Functional complex formation assays between the full length protease-helicase and RNA duplex substrates can also be performed by the method described by Ding et al. (Ding, S. C., et al. (2011) J. Virol. 85(9), 4343-4353).

### Example D

### Biological Activities of Combinations of Compounds of the Invention with Other Active Agents

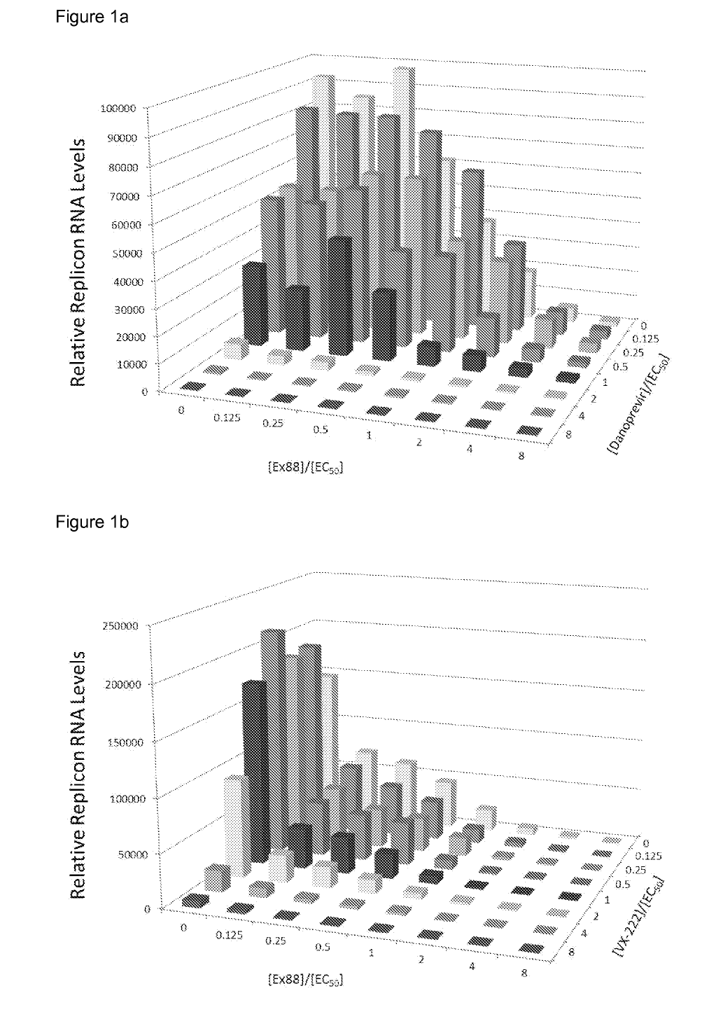

The replicon assay described in Example B above can be used to determine the reduction in HCV RNA load arising from the use of combinations of compounds of the invention with other active agents. The methods used differed from those set out in Example B only with regard to the compound concentrations tested, where the tested compounds are combined in an 8×8 matrix array using concentrations of 0, 0.125, 0.25, 0.5, 1.0, 2.0, 4.0, and 8.0× the pre-determined EC₅₀ of each respective compound tested. The EC₅₀s of the compounds of Examples 88 and 238B, Danoprevir and VX-222 were set as 300 nM, 30 nM, 1.0 nM, and 3.0 nM respectively, in line with previous observations. Lower luminescence values, as a read-out for lower HCV replicon RNA levels were observed in a dose dependent fashion for all of the HCV inhibitors in combination with other compounds tested here (FIGS. 1a-d below). Synergy plots generated from this data using the Bliss Independence Model also demonstrated additivity or synergy for all compound combinations tested.

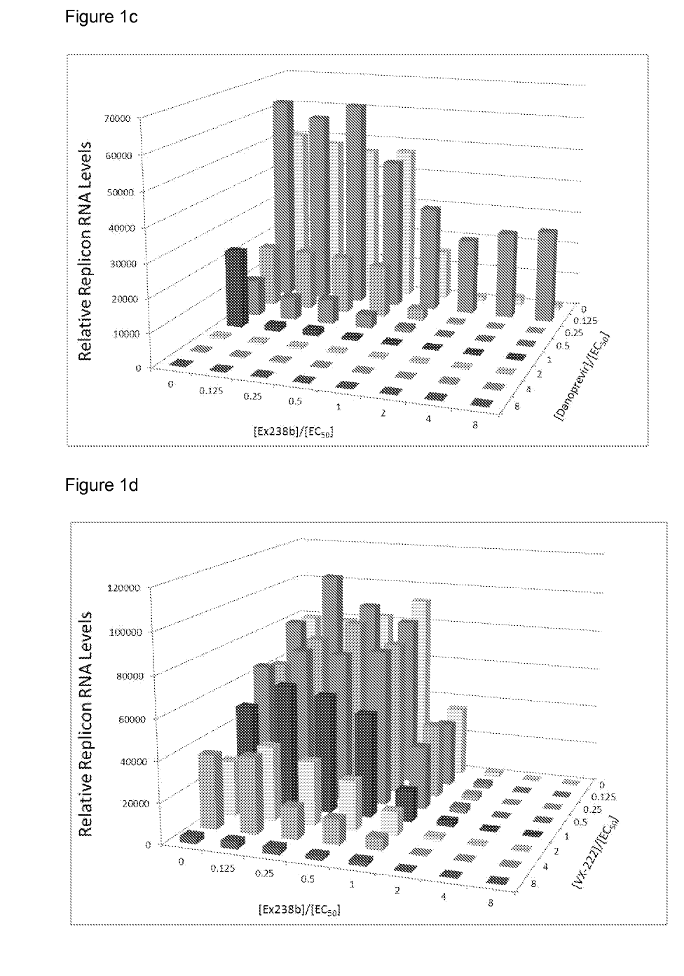

In order to directly determine HCV replicon RNA levels in HCV replicon bearing Huh-7 cells the cells were seeded at 100,000 cellswell, in 6 well tissue culture plates, and allowed to attach overnight before compound addition at a final DMSO concentration of 0.1%. At 72 hours post compound addition RNA was extracted from DMSO-only treated and compound-treated cells using a Qiagen RNeasy kit (Qiagen) according to the manufacturer's instructions. All samples were then normalized for total RNA concentration. Quantitative RT-PCR analysis was then carried out using the HCV NS5B gene specific primers: HCV5BF: CTCCATGGCCTTAGCGCATTT and HCV5BR: AAAAAACAGGATGGCCTATTGG in a one step reaction using the Quantitect SYBR Green RT-PCR kit (Qiagen) following the manufacturer's instructions. Briefly, sample RNA (2 ng) was combined with the NS5A primers listed above at a final concentration of 1 μM and an equal volume of 2× Quantitect SYBR Green RT-PCR Master Mix. Reactions were transferred to a thin walled 96 well plate and the RT reaction was carried out using the MX3005p (Stratagene) instrument at 50° C. for 30 minutes, followed by a denaturation step at 94° C. for 15 min. The PCR amplification was conducted in 45 cycles, each of which was 94° C. for 15 s followed by 59° C. for 30 seconds, then 72° C. for 2 minutes. Amplification of HPRT RNA for each sample was determined in separate reactions. The amount of input RNA from the untreated control sample was varied in order to generate a standard curve by which the relative levels of replicon RNA from each treated sample could be expressed as fold changes relative to the untreated control sample. HCV replicon GT1b levels reported as a log₁₀ reduction from the untreated control. Values were calculated from the average of three independent experiments, where log₁₀ HCVGAPDH levels at day 3, 7, 10, and 14 post compound treatment were subtracted from log₁₀ HCVGAPDH RNA levels of the untreated control. Samples were treated at 10× the EC₅₀ of the stated compound used as indicated for the length of time indicated (FIG. 2). The decline in HCV replicon RNA with the compound of Example 88 was comparable over time with Danoprevir and VX-222. The largest declines in HCV RNA replicon RNA were observed in samples treated with the compound of Example 88 in combination with either Danoprevir or VX-222.

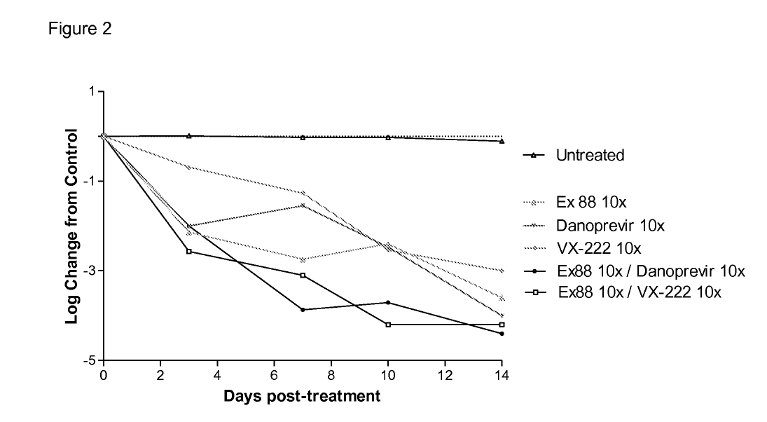

The existence of compound resistant HCV replicon quasispecies was analysed using colony forming assays, where the emergence of compound resistant HCV replicon variants can allow production sufficient replicon encoded neomycin for cellular survival in medium containing 1 mgml Gentamicin (Life Technologies). 4,000 replicon bearing cells were platedwell on 12 well plates, or 20,000 replicon bearing cellswell in 10 cm dishes, and allowed to adhere overnight. Compounds were then added at the indicated concentrations either alone or in combination at 0.1% DMSO final concentration. The medium used also contained 1 mgml geneticin. Plates were then incubated at 37° C. in an atmosphere of 5% CO2 and air for 24 days the mediumcompound solution with 1 mgml geneticin was replaced twice every 7 days, before staining surviving colonies with coomasie blue (FIG. 3 a-d). The emergence of compound resistant colonies was more efficiently eliminated with compound combinations than with the use of any tested compound alone.

### Example E

### Pharmaceutical Formulations

### (i) Tablet Formulation

A tablet composition containing a compound of the formula (1) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (1) with 100 mg lactose and filling the resulting mixture into standard opaque hard gelatin capsules.

(iii) Injectable Formulation I

A parenteral composition for administration by injection can be prepared by dissolving a compound of the formula (1) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (1) (e.g. in salt form) (2 mgml) and mannitol (50 mgml), sterile filtering the solution and filling into sealable 1 ml vials or ampoules.

### v) Injectable Formulation III

A formulation for i.v. delivery by injection or infusion can be prepared by dissolving the compound of formula (1) (e.g. in a salt form) in water at 20 mgml. The vial is then sealed and sterilised by autoclaving.

### vi) Injectable Formulation IV

A formulation for i.v. delivery by injection or infusion can be prepared by dissolving the compound of formula (1) (e.g. in a salt form) in water containing a buffer (e.g. 0.2 M acetate pH 4.6) at 20 mgml. The vial is then sealed and sterilised by autoclaving.

(vii) Subcutaneous Injection Formulation

A composition for sub-cutaneous administration is prepared by mixing a compound of the formula (1) with pharmaceutical grade corn oil to give a concentration of 5 mgml. The composition is sterilised and filled into a suitable container.

viii) Lyophilised Formulation

Aliquots of formulated compound of formula (I) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A compound of the formula (6): 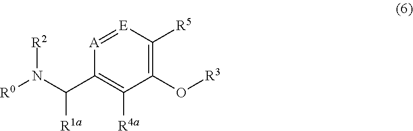
or a salt, N-oxide or tautomer thereof, wherein:
A is CH or CF;
E is CH or CF;
R⁰ is hydrogen or C₁₋₂ alkyl;
R^{1a} is selected from;
CONH₂;
CO₂H;
an acyclic C₁₋₈ hydrocarbon group optionally substituted with one or two substituents R⁶, wherein one carbon atom of the acyclic C₁₋₈ hydrocarbon group may optionally be replaced by a heteroatom or group selected from O, S, NR^{c}, S(O) and SO₂, or two adjacent carbon atoms of the acyclic C₁₋₈ hydrocarbon group may optionally be replaced by a group selected from CONR^{c}, NR^{c}CO, NR^{c}SO₂ and SO₂NR^{c} provided that in each case at least one carbon atom of the acyclic C₁₋₈ hydrocarbon group remains; and
a monocyclic carbocyclic or heterocyclic group of 3 to 7 ring members, of which 0, 1, 2, 3 or 4 are heteroatom ring members selected from O, N and S, the carbocyclic or heterocyclic group being optionally substituted with one or two substituents R^{7a};
R² is selected from hydrogen and a group R^{2a};
R^{2a} is selected from an acyclic C₁₋₈ hydrocarbon group optionally substituted with one or two substituents R⁸; a monocyclic carbocyclic or heterocyclic group of 5 or 6 ring members, of which 0, 1 or 2 ring members are heteroatom ring members selected from O and N; and a bicyclic heterocyclic group of 9 or 10 ring members, of which 1 or 2 ring members are nitrogen atoms, one of the rings of the bicyclic heterocyclic group being a benzene ring and the other of the rings being a 5 or 6 membered non-aromatic heterocyclic ring; the monocyclic carbocyclic or heterocyclic group and the bicyclic heterocyclic group each being optionally substituted with one or two substituents R^{7b};
wherein at least one of R¹ and R² is other than hydrogen;
R³ is a 3- to 10-membered monocyclic or bicyclic carbocyclic or heterocyclic ring containing 0, 1, 2 or 3 heteroatom ring members selected from N, O and S, and being optionally substituted with one or more substituents R¹³;
R^{4a} is selected from halogen; cyano; C₁₋₄ alkyl optionally substituted with one or more fluorine atoms; C₁₋₄ alkoxy optionally substituted with one or more fluorine atoms;
hydroxy-C₁₋₄ alkyl; and C₁₋₂ alkoxy-C₁₋₄ alkyl;
R⁵ is selected from hydrogen and a substituent Rya;
R^{5a} is selected from C₁₋₂ alkyl optionally substituted with one or more fluorine atoms; C₁₋₃ alkoxy optionally substituted with one or more fluorine atoms; halogen; cyclopropyl; cyano; and amino;
R⁶ is selected from hydroxy; fluorine; carbamoyl; mono- or di-C₁₋₄ alkylcarbamoyl; nitro; amino; mono- or di-C₁₋₄ alkylamino; a monocyclic carbocyclic or heterocyclic group of 3 to 7 ring members, of which 0, 1 or 2 are heteroatom ring members selected from O, N and S, the carbocyclic or heterocyclic group being optionally substituted with one or two substituents R^{7c};
R^{7a}, R^{7b}, R^{7c}, R^{7d}, R^{7e} and R^{7f} are each independently selected from oxo; amino; halogen; cyano; hydroxy; C₁₋₄ alkyl; hydroxy-C₁₋₄ alkyl; amino-C₁₋₄ alkyl; mono- and di-C₁₋₄ alkylamino-C₁₋₄ alkyl;
R⁸ is selected from hydroxy; halogen; cyano; C(═NH)NHR⁹; C(═O)NR¹⁰R¹¹; amino;
mono- or di-C₁₋₄ alkylamino; a non-aromatic monocyclic carbocyclic or heterocyclic group of 3 to 7 ring members, of which 0, 1 or 2 are heteroatom ring members selected from O, N and S, the carbocyclic or heterocyclic group being optionally substituted with 1 or 2 substituents R^{7d}; and an aromatic heterocyclic group selected from pyrrole, imidazole, pyrazole, indole and pyridone, the aromatic heterocyclic group being optionally substituted with 1 or 2 substituents R^{7e}; provided that the carbon atom of the acyclic C₁₋₈ hydrocarbon group which is attached directly to the moiety NR⁰ cannot be substituted with hydroxy or an N-linked substituent;
R⁹ is selected from hydrogen, C₁₋₄ alkyl and C₁₋₄ alkanoyl;
R¹⁰ is selected from hydrogen and C₁₋₄ alkyl;
R¹¹ is selected from hydrogen; hydroxy; C₁₋₄ alkoxy; amino; mono- or di-C₁₋₄ alkylamino; a monocyclic non-aromatic carbocyclic or heterocyclic group of 3 to 7 ring members, of which 0, 1 or 2 are heteroatom ring members selected from O, N and S, the non-aromatic carbocyclic or heterocyclic group being optionally substituted with one or two substituents R^{7f}; and C₁₋₆ alkyl, wherein the C₁₋₆ alkyl is optionally substituted with 1, 2 or 3 substituents R¹²;
or NR¹⁰R¹¹ forms a non-aromatic heterocyclic ring having a total of 4 to 7 ring members of which 1 or 2 are nitrogen atoms and the others are carbon atoms, the said non-aromatic heterocyclic ring being optionally substituted with one or more substituents selected from hydroxy, amino and C₁₋₄ alkyl;
R¹² is selected from hydroxy; C₁₋₄ alkoxy; cyano; C₁₋₄alkoxycarbonyl; amino; mono- or di-C₁₋₄ alkylamino; C₃₋₆cycloalkylamino; CONH₂; CONH(C₁₋₄alkyl); CON(C₁₋₄alkyl)₂ and a group —NH—CH₂-Cyc; where Cyc is a benzene, furan, thiophene or pyridine ring;
R¹³ is selected from halogen; cyano; nitro; CH═NOH; and a group R^{a}-R^{b}; and is optionally further selected from oxo;
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is hydrogen; a cyclic group R^{d}; or an acyclic C₁₋₈ hydrocarbon group optionally substituted with one or more substituents selected from hydroxy, oxo, halogen, cyano, nitro, carboxy, amino, mono- or di-C₁₋₄ alkylamino, and a cyclic group R^{d}; wherein one or two but not all of the carbon atoms of the acyclic C₁₋₈ hydrocarbon group may optionally be replaced by O, S, SO, SO₂, NR^{c}, X¹C(X²), C(X²)X¹ or X¹C(X²)X¹; SO₂NR^{c} or NR^{c}SO₂; the cyclic group R^{d} is a monocyclic carbocyclic or heterocyclic group having from 3 to 7 ring members, of which 0, 1, 2 or 3 are heteroatom ring members selected from O, N and S and oxidised forms thereof, the carbocyclic or heterocyclic group being optionally substituted with one or more substituents selected from R¹⁴; but excluding the combination wherein R^{a} is a bond and R^{b} is hydrogen;
R¹⁴ is selected from oxo; halogen; cyano; and R^{a}-R^{e};
R^{e} is hydrogen or an acyclic C₁₋₈ hydrocarbon group optionally substituted with one or more substituents selected from phenyl; hydroxy; oxo; halogen; cyano; carboxy; amino; mono- or di-C₁₋₄ alkylamino; wherein one or two but not all of the carbon atoms of the acyclic C₁₋₈ hydrocarbon group may optionally be replaced by O, S, SO, SO₂, NR^{c}, X¹C(X²), C(X²)X¹ or X¹C(X²)X¹; SO₂NR^{c} or NR^{c}SO₂;
X¹ is O or NR^{c};
X² is ═O or ═NR^{c}; and
R^{c} is hydrogen or C₁₋₄ alkyl.

2. A compound according to claim 1, or a salt, N-oxide or tautomer thereof, wherein A is CH and E is CH.

3. A compound according to claim 1, or a salt, N-oxide or tautomer thereof, wherein R⁰ is hydrogen.

4. A compound according to claim 1, or a salt, N-oxide or tautomer thereof, wherein R^{1a} is selected from:
an acyclic C₁₋₈ hydrocarbon group optionally substituted with one substituent R⁶, wherein one carbon atom of the acyclic C₁₋₈ hydrocarbon group may optionally be replaced by a heteroatom O; and
a monocyclic carbocyclic or heterocyclic group of 3, 4, 5 or 6 ring members, of which 0, 1 or 2 are heteroatom ring members selected from O and N, the carbocyclic or heterocyclic group being optionally substituted with one or two substituents R^{7a}.

5. A compound according to claim 4, or a salt, N-oxide or tautomer thereof, wherein R^{1a} is ethyl.

6. A compound according to claim 1, or a salt, N-oxide or tautomer thereof, wherein R² is selected from hydrogen and a group R^{2a} wherein R^{2a} is selected from a C₁₋₈ alkyl group optionally substituted with a substituent R⁸; cyclohexyl substituted with a substituent R^{7b}; pyridine optionally substituted with a substituent R^{7b}; and tetrahydroisoquinoline; wherein the substituent R⁸ is selected from hydroxy; C(═O)NR¹⁰R¹¹; piperidine; pyrrole and imidazole.

7. A compound according to claim 6, or a salt, N-oxide or tautomer thereof, wherein R² is a group R^{2a} wherein R^{2a} is a C₁₋₈ alkyl group optionally substituted with a substituent R⁸; wherein the substituent R⁸ is selected from hydroxy and C(═O)NR¹⁰R¹¹.

8. A compound according to claim 6, or a salt, N-oxide or tautomer thereof, wherein R² is hydrogen.

9. A compound according to claim 1, or a salt, N-oxide or tautomer thereof, wherein R^{4a} is fluorine.

10. A compound according to claim 1, or a salt, N-oxide or tautomer thereof, wherein R⁵ is fluorine or chlorine.

11. A compound according to claim 1, or a salt, N-oxide or tautomer thereof, wherein R³ is selected from 6-membered monocyclic aryl and heteroaryl groups containing 0, 1 or 2 nitrogen ring members and being optionally substituted with one or more substituents R¹³; 9-membered bicyclic heteroaryl groups containing 1, 2, 3 or 4 heteroatom ring members selected from O, N and S and being optionally substituted with one or more substituents R¹³; 9- and 10-membered partially aromatic bicyclic heterocyclic groups containing a benzene ring fused to a non-aromatic 5- or 6-membered heterocyclic ring containing 1 or 2 heteroatoms selected from O, N and S, the said partially aromatic bicyclic heterocyclic groups being optionally substituted with one or more substituents selected from oxo and R¹³.

12. A compound according to claim 11, or a salt, N-oxide or tautomer thereof, wherein R³ is selected from phenyl and pyridyl, each being optionally substituted with one or more substituents R¹³; and 9-membered partially aromatic bicyclic heterocyclic groups containing a benzene ring fused to a non-aromatic 5-membered heterocyclic ring containing 1 or 2 heteroatoms selected from O and N, the said partially aromatic bicyclic heterocyclic groups being unsubstituted or being substituted with one or two substituents selected from C₁₋₄ alkyl;.

13. A compound according to claim 1, or a salt, N-oxide or tautomer thereof, wherein the substituents R¹³ are selected from halogen; cyano; nitro; CH═NOH; and a group R^{a}-R^{b};
R^{a} is a bond, O, CO, X¹C(X²), C(X²)X¹, SO₂, NR^{c}, SO₂NR^{c} or NR^{c}SO₂;
R^{b} is hydrogen; a cyclic group R^{d}; or an acyclic C₁₋₈ hydrocarbon group optionally substituted with one or more substituents selected from hydroxy, oxo, halogen, cyano, amino, mono- or di-C₁₋₄ alkylamino, and a cyclic group R^{d}; wherein one or two but not all of the carbon atoms of the acyclic C₁₋₈ hydrocarbon group may optionally be replaced by O, NR^{c}, X¹C(X²), C(X²)X¹ or X¹C(X²)X¹; SO₂NR^{c} or NR^{c}SO₂ and wherein the cyclic group R^{d} is a monocyclic carbocyclic or heterocyclic group having from 3 to 7 ring members, of which 0, 1, 2 or 3 are heteroatom ring members selected from O and N, the carbocyclic or heterocyclic group being optionally substituted with one or more substituents selected from R¹⁴; but excluding the combination wherein R^{a} is a bond and R^{b} is hydrogen;
R¹⁴ is selected from cyano; and R^{a}-R^{e};
R^{e} is hydrogen or an acyclic C₁₋₈ hydrocarbon group optionally substituted with one or more substituents selected from phenyl and hydroxy
X¹ is O or NR^{c};
X² is ═O or ═NR^{c}; and
R^{c} is hydrogen or C₁₋₄ alkyl.

14. A compound according to claim 1 having the isomeric form (6a): 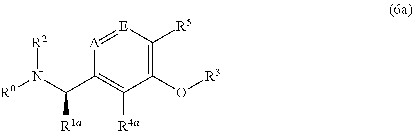
or a salt, N-oxide or tautomer thereof, wherein A, E, R⁰, R^{1a}, R², R³, R^{4a} and R⁵ are as defined in claim 1.

15. A compound according to claim 14 having the formula (2a): 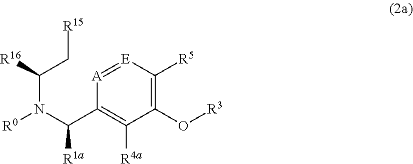
or a salt, N-oxide or tautomer thereof, wherein:
R¹⁵ is selected from hydrogen; a substituent R⁸; an acyclic C₁₋₃ hydrocarbon group optionally substituted with one or two substituents R⁸ wherein one carbon atom of the acyclic C₁₋₃ hydrocarbon group may optionally be replaced by a heteroatom or group selected from O and NR^{c} provided that at least one carbon atom of the acyclic C₁₋₃ hydrocarbon group remains; a monocyclic carbocyclic or heterocyclic group of 3 to 7 ring members, of which 0, 1 or 2 ring members are heteroatom ring members selected from O, N and S; and a bicyclic heterocyclic group of 9 or 10 ring members, of which 1 or 2 ring members are nitrogen atoms, one of the rings of the bicyclic heterocyclic group being a non-aromatic nitrogen-containing ring; the monocyclic carbocyclic or heterocyclic group and the bicyclic heterocyclic group each being optionally substituted with one or two substituents R^{7b};
R¹⁶ is selected from hydrogen and C₁₋₄ alkyl; and
A, E, R⁰, R^{1a}, R³, R^{4a}, R⁵ and R⁸ are as defined in claim 1.

16. A compound according to claim 1, or a salt, N-oxide or tautomer thereof, wherein
A is CH;
E is CH;
R⁰ is hydrogen or ethyl;
R^{1a} is selected from:
C₁₋₅ alkyl unsubstituted or substituted with a substituent selected from:
amino;
hydroxy;
methoxy;
fluorine;
isopropylamino;
pyridylaminocarbonyl; and
C(O)NH₂;
tetrahydropyridyl;
pyridyl;
piperidinyl;
piperidinylmethyl;
piperidinyl;
cyclohexenyl;
cyclopropyl;
tetrahydrofuranyl;
tetrahydropyranyl;
tetrahydropyranylmethyl; and
dihydroimidazolyl;
R² is selected from hydrogen and Rea;
R^{2a} is selected from:
C₁₋₃ alkyl optionally substituted with:
pyrrolyl;
pyrazolyl;
imidazolyl wherein the imidazolyl is optionally substituted with one or two methyl or ethyl groups;
cyclopropyl;
azetidinyl;
piperidinyl;
indolyl;
pyridyl;
hydroxy;
SH; and
cyano;
allyl;
dihydroxypropyl;
cyclobutyl;
cyclopentyl;
aminocyclohexyl;
aminocyclobutyl;
piperidinyl;
aminomethylpyrimidinyl;
CH(R¹⁷)(CH₂)ₐC(O)NR^{18a}R^{18b} where a is 0 or 1; R¹⁷ is hydrogen, C₁₋₃ alkyl or cyclopropyl; R¹⁸ is hydrogen or methyl and R^{18b} is selected from:
hydrogen;
methyl;
cyclopropyl;
amino-C₂₋₄ alkyl;
dimethylaminoethyl;
ethylaminoethyl;
cyanomethyl;
hydroxy-C₂₋₄ alkyl;
pyridyl;
CH₂C(O)OCH₃;
CH₂C(O)NH₂;
amino;
methoxy;
oxetanyl;
azetidinyl;
aminocyclobutyl;
pyrrolidinyl;
piperidinyl;
benzylaminoethyl;
or NR^{18a}R^{18b} forms a piperazine or diazepine ring;
pyridyl optionally substituted with amino;
tetrahydroisoquinolinyl;
dihydroisoindolyl; and
imidazolyl;
wherein at least one of R¹ and R² is other than hydrogen;
R³ is selected from:
unsubstituted phenyl;
phenyl substituted with one substituent selected from:
—(CH₂)_{y}NHSO₂CH₃ where y is 0 or 1;
ethyl;
hydroxymethyl;
hydroxyethyl;
methoxyethyl;
pyrrolidinylcarbonyl;
C(O)NHR¹⁹; where R¹⁹ is hydrogen or cyanoethyl;
C(O)NR²⁰R²¹ where R²⁰ is methyl and R²¹ is pyrazol-4-ylmethyl or 1-benzylpyrazol-4-ylmethyl;
—CH(CH₃)OC(O)NHCH₂CH₃;
CH₂OC(O)NHCH₂Cyp where Cyp is cyclopropyl;
fluorine;
chlorine;
nitro;
cyano;
dimethylamino;
cyanomethyl;
trifluoromethyl;
methylsulphonyl;
—NH(CO)NHCH₂CF₃;
—CH₂NHC(O)CH₃;
methyloxadiazolyl;
oxazolyl;
—SO₂NHCH₃;
cyanocyclopropyl;
hydroxymethylcyclopropyl;
CH═N—OH;
ethynyl;
disubstituted phenyl wherein the two substituents are selected from cyano, fluorine, chlorine, methyl, methoxy, nitro, oxazolyl, C(O)NH₂, trifluoromethyl, acetylamino and amino;
pyridine unsubstituted or substituted with a substituent selected from amino, acetylamino, chlorine, cyano, methyl, C(O)NH₂ and hydroxymethyl;
pyridazine substituted with chorine;
dihydrobenzofuran;
dihydroindole substituted with two methyl groups; and
pyridone;
R⁴ is selected from fluorine and chlorine; and
R⁵ is selected from fluorine; chlorine; methyl and ethyl.

17. A pharmaceutical composition comprising a compound as defined in claim 1, or a salt, N-oxide or tautomer thereof, and a pharmaceutically acceptable excipient.

18. (canceled)

19. A combination of a compound as defined in claim 1, or a salt, N-oxide or tautomer thereof, and (i) a further anti-hepatitis C virus agent or (ii) an anti-cancer agent

20. (canceled)

21. A compound according to claim 1 which is in the form of a salt.

22. A method of preventing or treating a hepatitis C virus infection in a subject, which method comprises administering to the subject an effective anti-hepatitis C viral amount of a compound as defined in claim 1, or a salt, N-oxide or tautomer thereof.
